(19) United States
(12) Patent Application Publication
(10) Pub. No.: US 2025/0221988 A1
(43) Pub. Date: Jul. 10, 2025

(54) COMPOUNDS AND METHODS OF TREATING CANCERS

(71) Applicant: Cullgen (Shanghai), Inc., Shanghai (CN)

(72) Inventors: Jing LIU, Oradell, NJ (US); Michael Bruno PLEWE, San Diego, CA (US); Jialiang WANG, Nashville, TN (US); Xiaoran HAN, Shanghai (CN); Ting YANG, Shanghai (CN); Chengwei ZHANG, Shanghai (CN); Liqun CHEN, Shanghai (CN); Lihuai QIN, Shanghai (CN)

(21) Appl. No.: 18/853,829

(22) PCT Filed: Apr. 6, 2023
(86) PCT No.: PCT/CN2023/086579

§ 371 Date: Oct. 3, 2024

(30) Foreign Application Priority Data
Apr. 6, 2022 (WO) PCT/CN2022/085329

(51) Int. Cl. A61K 31/498; A61K 31/4545; A61K 31/497; A61P 35/02; C07D 401/14; C07D 471/04

(52) U.S. Cl. CPC A61K 31/498; A61K 31/4545; A61K 31/497; A61P 35/02; C07D 401/14; C07D 471/04

(57) ABSTRACT

This disclosure relates to GSPT1 degrader compounds and pharmaceutically acceptable salts thereof, pharmaceutical compositions comprising such compounds or salts, and methods of making and using the compounds or salts for the treatment of certain diseases. The disclosure also relates to methods for identifying such compounds.

### BACKGROUND

There is a need in the art for compounds, compositions, and methods of use of the compounds for the treatment of diseases in a subject in need thereof.

### SUMMARY

This disclosure relates to compounds (e.g. small molecule compounds), compositions comprising one or more of the compounds, and to methods of use of the compounds for the treatment of certain diseases in a subject in need thereof. The disclosure also relates to methods for identifying or making such compounds.

In some embodiments, are methods of treatment, comprising: administering to a subject in need thereof, a first compound comprising a GSPT1 degrader and a second compound comprising an FLT3 pathway inhibitor, a RAS-RAF-MEK-ERK pathway inhibitor, or a PI3K-AKT-mTOR pathway inhibitor or activator.

In some embodiments, provided herein is a compound of Formula (A):

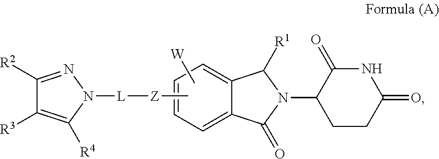

or a pharmaceutically acceptable salt thereof, wherein:
W is hydrogen or fluorine;
Z is absent, —NR^{1a}—, or —O—; wherein R^{1a} is hydrogen or C₁-C₈ alkyl;
L is an optionally substituted C₁-C₁₀ alkylene or optionally substituted C₁-C₁₀ heteroalkylene, or L is

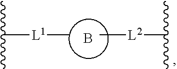

wherein
   Ring B is an optionally substituted 3-7 membered carbocyclyl or optionally substituted 4-7 membered heterocyclyl;
   L¹ is absent, or an optionally substituted C₁-C₁₀ alkylene or optionally substituted C₁-C₁₀ heteroalkylene;
   L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl, optionally substituted 6-10 membered aryl, or optionally substituted 5-10 membered heteroaryl; or
R² and R³ or R³ and R⁴ are taken together to form an optionally substituted 3-7 membered partially saturated or unsaturated carbocyclyl, optionally substituted 4-7 membered partially saturated or unsaturated heterocyclyl, optionally substituted phenyl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl or optionally substituted 5-6 membered heteroaryl ring.

In some embodiments, provided herein is a compound of Formula (I):

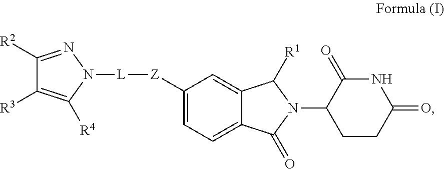

or a pharmaceutically acceptable salt thereof, wherein:
Z is absent, —NR^{1a}—, or —O—; wherein Ra is hydrogen or C₁-C₈ alkyl; L is an optionally substituted C₁-C₁₀ alkylene or optionally substituted C₁-C₁₀ heteroalkylene, or L is

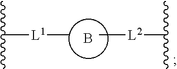

wherein
   Ring B is an optionally substituted 3-7 membered carbocyclyl or optionally substituted 4-7 membered heterocyclyl;
   L¹ is absent, or an optionally substituted C₁-C₁₀ alkylene or optionally substituted C₁-C₁₀ heteroalkylene;
   L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl, optionally substituted 6-10 membered aryl, or optionally substituted 5-10 membered heteroaryl; or
R² and R³ or R³ and R⁴ are taken together to form an optionally substituted 3-7 membered partially saturated or unsaturated carbocyclyl, optionally substituted 4-7 membered partially saturated or unsaturated heterocyclyl, optionally substituted phenyl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl or optionally substituted 5-6 membered heteroaryl.

In some embodiments of Formula (I), R² and R³ are taken together to form an optionally substituted phenyl or optionally substituted 5-6 membered heteroaryl ring.

In some embodiments of Formula (I), R³ and R⁴ are taken together to form an optionally substituted phenyl or optionally substituted 5-6 membered heteroaryl ring. In some such embodiments, R³ and R⁴ are taken together to form Ring A and provide the structure of Formula (II).

In some embodiments, the compound of Formula (I) has the structure of Formula (II):

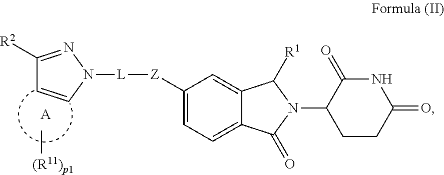

or a pharmaceutically acceptable salt thereof, wherein:
R¹, R², L and Z are defined as in Formula (I);
Ring A is a phenyl or 5-6 membered heteroaryl;
each R¹¹ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; and
p₁ is 0, 1, 2, 3, or 4.

In some embodiments, the compound of Formula (I) has the structure of Formula (III):

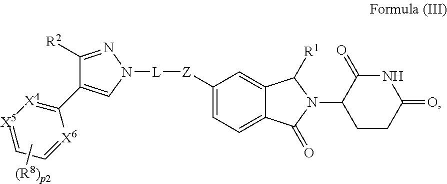

or a pharmaceutically acceptable salt thereof, wherein:
R¹, R², L and Z are defined as in Formula (I);
X⁴, X⁵ and X⁶ are each independently CR⁸ or N;
each R⁸ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
two R⁸ on adjacent carbon atoms are taken together to form an optionally substituted partially unsaturated 3-7 membered carbocyclyl, optionally substituted partially unsaturated 4-7 membered heterocyclyl, optionally substituted phenyl, or optionally substituted 5-6 membered heteroaryl; and
p₂ is 0, 1, 2, 3, 4, or 5.

In other embodiments, the compound of Formula (I) has the structure of Formula (IV):

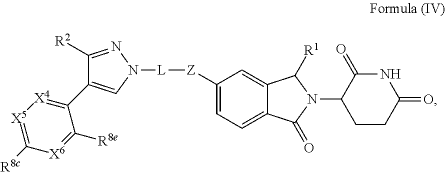

or a pharmaceutically acceptable salt thereof, wherein:
R¹, R², L and Z are defined as in Formula (I);
X⁴ is CR^{8a} or N;
X⁵ is CR^{8b} or N;
X⁶ is CR^{8d} or N;
R^{8a}, R^{8b}, R^{8c}, R^{8d}, and R^{8e} are each independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
R^{8a} and R^{8b}, R^{8b} and R^{8c}, R^{8c} and R^{8d}, or R^{8d} and R^{8e} are taken together with the atoms to which they are attached to form an optionally substituted 3-7 membered carbocyclyl, optionally substituted 4-7 membered heterocyclyl, optionally substituted phenyl, or optionally substituted 5-6 membered heteroaryl.

In another aspect, provided herein is a pharmaceutical composition comprising a compound of any of the formulae described herein, or a pharmaceutically acceptable salt thereof, and a pharmaceutically acceptable excipient

In another aspect, provided herein is a method of treatment, comprising administering an effective amount of the compound of any of the formulae described herein, or a pharmaceutically acceptable salt thereof, or the pharmaceutical composition comprising such a compound of salt, as described herein to a subject in need thereof. In some embodiments, the subject has cancer. In some embodiments, the method further comprises administering to the subject a second compound comprising an FLT3 pathway inhibitor, a RAS-RAF-MEK-ERK pathway inhibitor, or a PI3K-AKT-mTOR pathway inhibitor or activator. In some embodiments, the method further comprises administering to the subject a second compound comprising a chemotherapy reagent. Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-1B show small molecule-mediated targeted degradation of GSPT1. MOLM-13 cells were treated at indicated concentrations with compounds GS-729, GS-731, GS-697, and GS-788 for 16 hours prior to immunoblotting.

FIG. 2A-2C show FLT3 inhibition sensitizes AML cells to GSPT1 degraders GS-707, GS-749, and GS-750. MOLM-13 cells were treated with GSPT1 degraders at indicated concentrations following a 3-fold serial dilution with or without 20 nM gilteritinib for 3 days.

FIG. 3 shows pancreatic cell line KP-4 is sensitive to GSPT1 degraders GS-668, GS-676, and GS-766. KP-4 cells were treated with GSPT1 degraders at indicated concentrations following a 3-fold serial dilution for 3 days.

FIG. 4A-4B shows GSPT1 degraders reduce GSPT1 protein levels in xenograft tumors. (FIG. 4A) Nude mice bearing MOLM-13 or (FIG. 4B) 22RV1 xenograft tumors were orally treated with 30 mg/kg GSPT1 degraders or vehicle. Tumors were collected for immunoblotting.

FIG. 5 shows small molecule-mediated targeted degradation of GSPT1. MOLM-13 cells were treated at indicated concentrations with compounds GS-802 and GS-803 for 16 hours prior to immunoblotting.

FIG. 6 shows small molecule-mediated targeted degradation of GSPT1. 22RV1 cells were treated at indicated concentrations with compounds GS-807, GS-813 and GS-814 for 8 hours prior to immunoblotting.

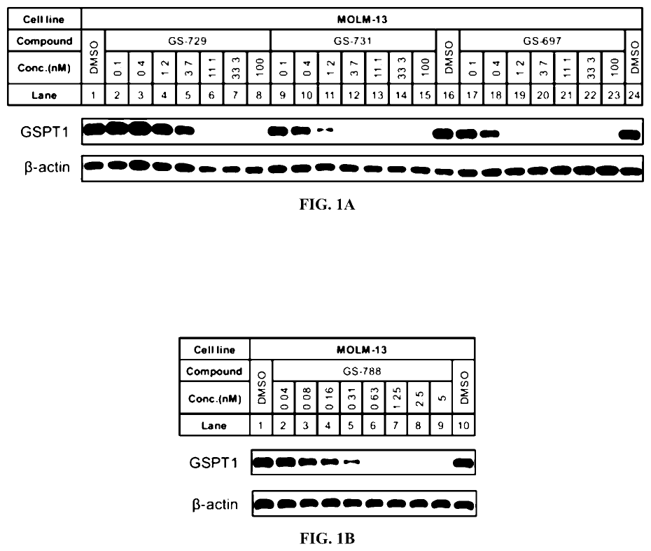

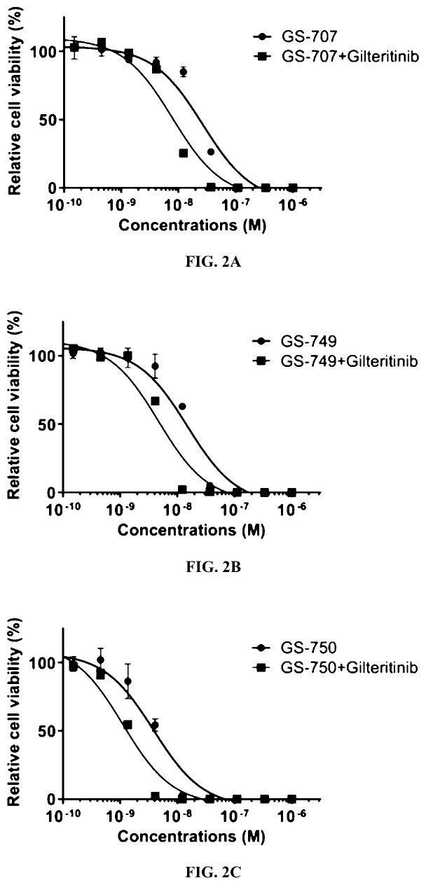

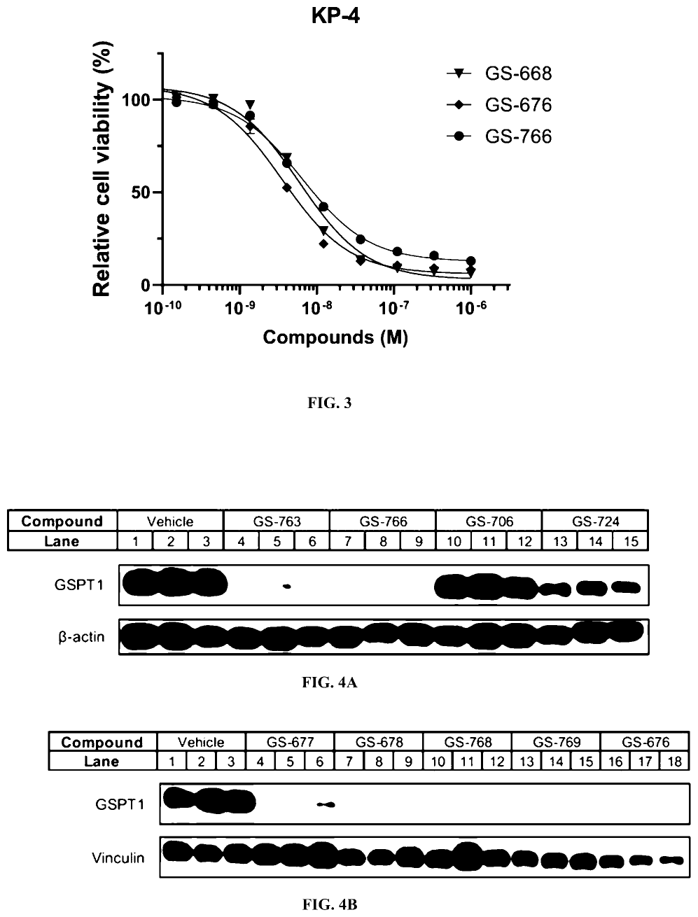

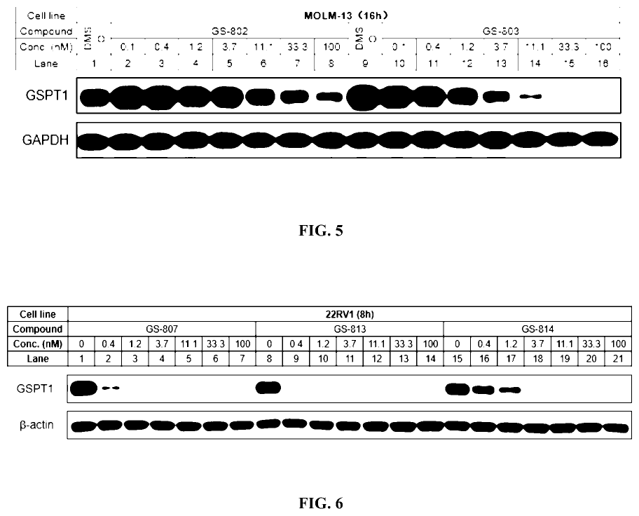

### DETAILED DESCRIPTION

Termination of translation is a GTP-dependent process that is regulated by two key proteins eRF1 and eRF3. The translation termination factor eRF3a (also known as eukaryotic peptide chain release factor GTP-binding subunit ERF3A, or “GSPT1”) is a GTPase that interacts with eRF1 to promote stop codon recognition and release of nascent peptide from ribosome (Chauvin, Salhi et al. 2005). GSPT1 activates eRF1 in a GTP-dependent manner and its GTPase activity requires complexing with eRF1 and ribosomes (Frolova, Le Goff et al. 1996). The GTP-bound GSPT1 and eRF1 together with ribosomes form the functional translation termination complexes (Zhouravleva, Frolova et al. 1995).

Through regulation of translation, GSPT1 has diverse and important roles in cell physiology. Increased expression of GSPT1 has been reported in human malignancies, including lung cancer and gastric cancer (Malta-Vacas, Aires et al. 2005, Tian, Tian et al. 2018, Sun, Zhang et al. 2019, Zhang, Zou et al. 2019).

Hence, GSPT1 is thought to be a novel cancer target through which one may compromise active translation that contributes to malignant phenotypes of cancer cells. Recently, Matyskiela and colleagues have reported that a phthalimide-derived molecule CC-885 led to cereblon-dependent degradation of GSPT1 and other targets, such as IKZF1 and IKZF3 (Matyskiela, Lu et al. 2016). Ishoey et al. also reported that GSPT1 was degraded by a subset of heterobiofunctional compounds derived from phthalimide (Ishoey, Chorn et al. 2018). CC-885 induced significant toxicity in the vast majority of tested cell lines, presumably due to degrading GSTP1 and many other proteins (Matyskiela, Lu et al. 2016). Therefore, despite the broad and potent anti-cancer activity, CC-885 exhibits unacceptable toxicity that prevents further development (Hansen, Correa et al. 2020).

Disclosed herein, in an aspect, are compounds. In some embodiments, the compound comprises a chemical structure or formula disclosed herein. The compound includes a GSPT1 degrader. GSPT1 degraders may be characterized by the ability to degrade or reduce cellular protein levels of GSPT1.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### 1. Compounds of the Disclosure

Disclosed herein, in some embodiments, are compounds. In some embodiments, the compound comprises a degradation tag. In some embodiments, the compound comprises a cereblon-binding moiety. In some embodiments, the degradation tag comprises the cereblon-binding moiety. In some embodiments, the compound comprises a GSPT1 degrader. For example, the compound may result in GSPT1 degradation. The compound may degrade GSPT1 as a result of cereblon modulation by the Degradation Tag. The compound may bind to or modulate GSPT1 or cereblon. In some embodiments, the compound comprises a heterobifunctional compound. In some embodiments, the compound comprises a molecular glue. In some embodiments, the compound may be used as a molecular glue. In some embodiments, the compound comprises a linker. In some embodiments, the compound comprises a truncated Janus kinase (JAK)-binding moiety.

In one aspect, provided herein is a compound of Formula (A):

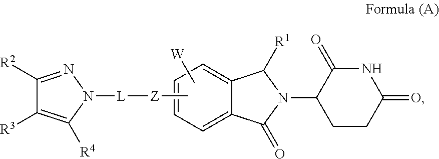

or a pharmaceutically acceptable salt thereof, wherein:
W is hydrogen or fluorine;
Z is absent, —NR^{1a}—, or —O—; wherein Ra is hydrogen or C₁-C₈ alkyl;
L is an optionally substituted C₁-C₁₀ alkylene or optionally substituted C₁-C₁₀ heteroalkylene, or
L is

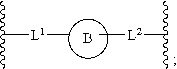

wherein
   Ring B is an optionally substituted 3-7 membered carbocyclyl or optionally substituted 4-7 membered heterocyclyl;
   L¹ is absent, or an optionally substituted C₁-C₁₀ alkylene or optionally substituted C₁-C₁₀ heteroalkylene;
   L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl, optionally substituted 6-10 membered aryl, or optionally substituted 5-10 membered heteroaryl; or
R² and R³ or R³ and R⁴ are taken together to form an optionally substituted 3-7 membered partially saturated or unsaturated carbocyclyl, optionally substituted 4-7 membered partially saturated or unsaturated heterocyclyl, optionally substituted phenyl, or optionally substituted 5-6 membered heteroaryl;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl or optionally substituted 5-6 membered heteroaryl.

In some embodiments of Formula (A), W is hydrogen. In some embodiments of Formula (A), W is fluorine.

In another aspect, provided herein is a compound of Formula (I):

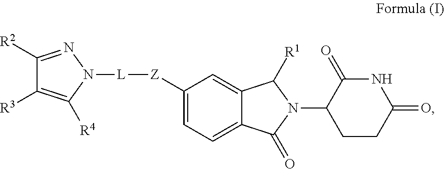

or a pharmaceutically acceptable salt thereof, wherein:
Z is absent, —NR^{1a}—, or —O—; wherein R^{1a} is hydrogen or C₁-C₈ alkyl;
L is an optionally substituted C₁-C₁₀ alkylene or optionally substituted C₁-C₁₀ heteroalkylene, or
L is

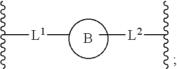

wherein
   Ring B is an optionally substituted 3-7 membered carbocyclyl or optionally substituted 4-7 membered heterocyclyl;
   L¹ is absent, or an optionally substituted C₁-C₁₀ alkylene or optionally substituted C₁-C₁₀ heteroalkylene;
   L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted saturated or partially unsaturated 3-10 membered carbocyclyl, or optionally substituted saturated or partially unsaturated 3-10 membered heterocyclyl, optionally substituted 6-10 membered aryl, or optionally substituted 5-10 membered heteroaryl; or
R² and R³ or R³ and R⁴ are taken together to form an optionally substituted partially unsaturated 3-7 membered carbocyclyl, optionally substituted partially unsaturated 4-7 membered heterocyclyl, optionally substituted phenyl, or optionally substituted 5-6 membered heteroaryl;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl or optionally substituted 5-6 membered heteroaryl.

In some embodiments of Formula (A) or Formula (I), R² and R³ are taken together to form an optionally substituted phenyl or optionally substituted 5-6 membered heteroaryl. In some embodiments, R² and R³ are taken together to form an optionally substituted phenyl. In some embodiments, R² and R³ are taken together to form an optionally substituted 5-6 membered heteroaryl. In some embodiments, R² and R³ are taken together to form an optionally substituted 6 membered heteroaryl comprising 1-3 heteroatoms selected from N and O. In some embodiments, the 6-membered heteroaryl is pyridinyl, or triazinyl. In some embodiments, the 6-membered heteroaryl is pyridinyl.

In some such embodiments, R² and R³ are taken together to form an optionally substituted partially unsaturated 3-7 membered carbocyclyl. In some embodiments, R² and R³ are taken together to form an optionally substituted partially unsaturated 4-7 membered heterocyclyl comprising 1-3 heteroatoms selected from N, O, and S. In some such embodiments, R² and R³ are taken together to form an optionally substituted partially unsaturated 3-7 membered carbocyclyl. In some embodiments, R² and R³ are taken together to form an optionally substituted partially unsaturated 4-7 membered heterocyclyl comprising 1-3 heteroatoms selected from N, O, and S.

In some embodiments of Formula (A) or Formula (I), R³ and R⁴ are taken together to form an optionally substituted phenyl or optionally substituted 5-6 membered heteroaryl. In some embodiments, R³ and R⁴ are taken together to form an optionally substituted phenyl. In some embodiments, R³ and R⁴ are taken together to form an optionally substituted 5-6 membered heteroaryl. In some embodiments, R³ and R⁴ are taken together to form an optionally substituted 6-membered heteroaryl. In some embodiments, the 6-membered heteroaryl contains 1-3 heteroatoms selected from N and O. In some embodiments, the 6-membered heteroaryl is pyridinyl, or triazinyl. In some embodiments, the 6-membered heteroaryl is pyrimidinyl, pyrazinyl, or pyridazinyl.

In some such embodiments, R³ and R⁴ are taken together to form an optionally substituted partially unsaturated 3-7 membered carbocyclyl. In some embodiments, R³ and R⁴ are taken together to form an optionally substituted partially unsaturated 4-7 membered heterocyclyl comprising 1-3 heteroatoms selected from N, O, and S.

Embodiments described herein for Formula (A) or Formula (I) are also applicable for any of Formulae (II), (III) or (IV) (including for clarity (II-A), (IV-A), (IV-B), (IV-C) or (IV-D)), to the extent that such embodiments are not inconsistent.

In some embodiments of Formula (I) when R³ and R⁴ are taken together to form an optionally substituted phenyl or optionally substituted 5-6 membered heteroaryl ring, such ring corresponds to Ring A in compounds of Formula (II).

In some embodiments, the compound of Formula (I) has the structure of Formula (II):

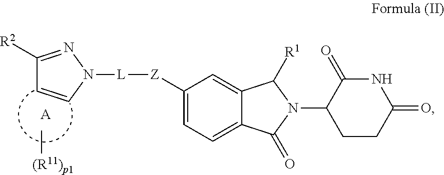

or a pharmaceutically acceptable salt thereof, wherein:
R¹, R², L and Z are defined as in Formula (I);
Ring A is a phenyl or 5-6 membered heteroaryl;
each R¹¹ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; and
p₁ is 0, 1, 2, 3, or 4.

In some embodiments of Formula (II), Ring A is phenyl, pyridinyl, or triazinyl. In some embodiments, Ring A is phenyl. In some embodiments, Ring A is pyridinyl. In some embodiments, Ring A is triazinyl. In some embodiments of Formula (II), Ring A is pyrimidinyl, pyrazinyl, or pyridazinyl. In some embodiments, Ring A is pyrimidinyl. In some embodiments, Ring A is pyrazinyl. In some embodiments, Ring A is pyridazinyl.

Embodiments described herein for Formula (II) are also applicable for Formula (II-A), to the extent that such embodiments are not inconsistent.

In some embodiments, the compound of Formula (II) has the structure of Formula (II-A):

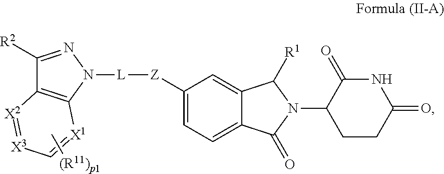

or a pharmaceutically acceptable salt thereof, wherein:
R¹, R², L and Z are defined as in Formula (I); and
X¹, X² and X³ are each independently CR¹¹ or N.

In some embodiments of Formula (II-A), X¹, X², and X³ are each independently CR¹¹.

In some embodiments of Formula (II-A), X¹, X², and X³ are each independently N.

In some embodiments of Formula (II-A), X¹ is N; and X² and X³ are each independently CR¹¹.

In some embodiments of Formula (II-A), X¹ and X² are each N; and X³ is CR¹¹.

In some embodiments of Formula (II-A), X¹ and X³ are each N; and X² is CR¹¹.

In some such embodiments, each R¹¹ is independently hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some embodiments, each R¹¹ is independently hydrogen, halogen, optionally substituted C₁-C₈ alkyl. In some embodiments, each R¹¹ is independently hydrogen. In some embodiments, each R¹¹ is independently methyl, ethyl, n-propyl, iso-propyl, or tert-butyl. In some embodiments, each R¹¹ is independently methyl. In some embodiments, each R¹¹ is independently an optionally substituted 3-10 membered carbocyclyl or optionally substituted 3-10 membered heterocyclyl. In some embodiments, each R¹¹ is independently optionally substituted cyclopropyl, optionally substituted cyclobutyl, optionally substituted cyclopentyl, or optionally substituted cyclohexyl. In some embodiments, each R¹¹ is independently cyclopropyl, cyclobutyl, cyclopentyl, or cyclohexyl. In some embodiments, each R¹¹ is independently oxetane, tetrahydrofuran, tetrahydro-2H-pyran, pyrrolidine, piperidine, morpholine, or piperazine. In some embodiments, each R¹¹ is independently hydrogen, Cl, F, Br, CN, NO₂, OH, OCH₃, methyl, ethyl, or iso-propyl. In some embodiments, each R¹¹ is independently hydrogen, Cl, F, Br, CN, NO₂, NH₂, OH, OCH₃, methyl, ethyl, or iso-propyl. In some embodiments, each R¹¹ is independently hydrogen, Cl, F, Br, OH, OCH₃, methyl, or iso-propyl. In some embodiments, each R¹¹ is independently hydrogen, Cl, F, Br, NH₂, OH, OCH₃, methyl, or iso-propyl. In some embodiments, each R¹¹ is OCH₃. In some embodiments, each R¹¹ is N(R⁵)R⁶. In some embodiments, each R¹¹ is NH₂. In some embodiments, each R¹¹ is N(R⁵)R⁶, wherein R⁵ and R⁶ are taken together with the N to which they are attached to form a 3-10 membered heterocyclyl. In some embodiments, each R¹¹ is N(R⁵)R⁶, wherein R⁵ and R⁶ are taken together with the N to which they are attached to form a piperazine.

In some embodiments of Formula (A) or Formula (I), R³ is halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted saturated or partially unsaturated 3-10 membered carbocyclyl, or optionally substituted saturated or partially unsaturated 3-10 membered heterocyclyl, optionally substituted 6-10 membered aryl, or optionally substituted 5-10 membered heteroaryl.

In some such embodiments, R³ is N(R⁵)R⁶, optionally substituted 3-10 membered carbocyclyl, optionally substituted 3-10 membered heterocyclyl, optionally substituted 6-10 membered aryl, or optionally substituted 5-10 membered heteroaryl. In some embodiments, R³ is an optionally substituted 6-10 membered aryl or optionally substituted 5-10 membered heteroaryl. In some embodiments, R³ is an optionally substituted phenyl or optionally substituted 6-10 membered heteroaryl. In some embodiments, R³ is a substituted phenyl. In some embodiments, R³ is an optionally substituted 5-10 membered heteroaryl. In some embodiments, R³ is optionally substituted 6-10 membered heteroaryl. In some embodiments, R³ is substituted 6-10 membered heteroaryl. In some such embodiments, the 6-10 membered heteroaryl is selected from pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, benzofurazanyl, benzothiophenyl, benzothiazolyl, benzoxazolyl, quinazolinyl, quinoxalinyl, naphthyridinyl, and furopyridinyl. In some such embodiments, the 6-10 membered heteroaryl is pyridinyl, pyrimidinyl, or pyrazinyl. In some such embodiments, the 6-10 membered heteroaryl is pyridinyl. In some such embodiments, the 6-10 membered heteroaryl is selected from pyrazolopyridinyl, imidazopyridinyl, tetrahydronaphthyridinyl, pyrrolopyridinyl, and dihydrocyclopentapyridinyl. In some such embodiments, the 6-10 membered heteroaryl is pyrazolopyridinyl. In some such embodiments, the 6-10 membered heteroaryl is imidazopyridinyl. In some such embodiments, the 6-10 membered heteroaryl is pyrrolopyridinyl.

In some embodiments of Formula (A) or Formula (I), R³ is hydrogen.

In some embodiments of Formula (A) or Formula (I), R³ is optionally substituted C₁-C₈ alkyl. In some embodiments of Formula (A) or Formula (I), R³ is optionally substituted C₁-C₃ alkyl. In some embodiments of Formula (A) or Formula (I), R³ is optionally substituted methyl. In some embodiments of Formula (A) or Formula (I), R³ is benzyl.

In some embodiments of Formula (A) or Formula (I), R³ is optionally substituted 3-10 membered carbocyclyl. In some embodiments of Formula (A) or Formula (I), R³ is optionally substituted

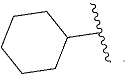 [SMILES: *C1CCCCC1]

In some embodiments of Formula (A) or Formula (I), R³ is

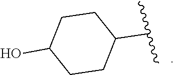 [SMILES: *C1CCC(O)CC1]

In some embodiments of Formula (A) or Formula (I), R³ is

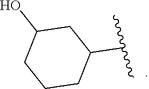 [SMILES: *C1CCCC(O)C1]

In some such embodiments, R³ is N(R⁵)R⁶. In some embodiments, R³ is NHR⁶. In some embodiments, R³ is NHR⁶, wherein R⁶ is optionally substituted 3-10 membered carbocyclyl. In some embodiments, R³ is optionally substituted

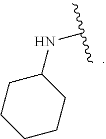 [SMILES: *NC1CCCCC1]

In some such embodiments, R³ is

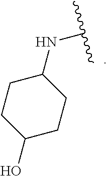 [SMILES: *NC1CCC(O)CC1]

In some embodiments, R³ is NHR⁶, wherein R⁶ is optionally substituted 3-10 membered heterocyclyl. In some embodiments, R³ is NHR⁶, wherein R⁶ is optionally substituted 6 membered heterocyclyl. In some embodiments, R³ is optionally substituted

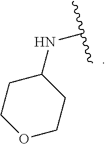 [SMILES: *NC1CCOCC1]

In some embodiments, R³ is optionally substituted

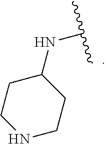 [SMILES: *NC1CCNCC1]

In some embodiments, R³ is

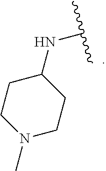 [SMILES: *NC1CCN(C)CC1]

In some embodiments, R³ is NHR⁶, wherein R⁶ is optionally substituted 6-10 membered aryl. In some embodiments, R³ is NHR⁶, wherein R⁶ is optionally substituted phenyl. In some embodiments, R³ is NHR⁶, wherein R⁶ is optionally substituted 5-10 membered heteroaryl. In some embodiments, R³ is NHR⁶, wherein R⁶ is optionally substituted 5 or 6 membered heteroaryl. In some embodiments, R³ is NHR⁶, wherein R⁶ is optionally substituted 5 or 6 membered heteroaryl comprising 1, 2, or 3 nitrogens. In some embodiments, R³ is NHR⁶, wherein R⁶ is optionally substituted 5 membered heteroaryl. In some embodiments, R³ is optionally substituted

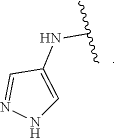 [SMILES: *Nc1cn[nH]c1]

In some embodiments, R³ is

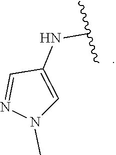 [SMILES: *Nc1cnn(C)c1]

In some embodiments, R³ is NHR⁶, wherein R⁶ is optionally substituted 6 membered heteroaryl. In some embodiments, R³ is optionally substituted

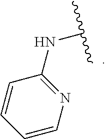 [SMILES: *Nc1ccccn1]

In some embodiments, R³ is optionally substituted

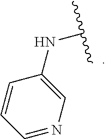 [SMILES: *Nc1cccnc1]

In some embodiments, R³ is optionally substituted

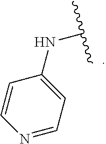 [SMILES: *Nc1ccncc1]

In some embodiments of Formula (A) or Formula (I), R⁴ is hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some embodiments, R⁴ is halogen, CN, OR⁵, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl. In some embodiments, R⁴ is hydrogen, halogen, optionally substituted C₁-C₈ alkyl, or optionally substituted 3-10 membered carbocyclyl. In some embodiments, R⁴ is methyl, ethyl, n-propyl, iso-propyl, or tert-butyl. In some embodiments, R⁴ is substituted methyl. In some embodiments, R⁴ is benzyl. In some embodiments, R⁴ is an optionally substituted 3-10 membered carbocyclyl. In some embodiments, R⁴ is optionally substituted cyclopropyl, optionally substituted cyclobutyl, optionally substituted cyclopentyl, or optionally substituted cyclohexyl. In some embodiments, R⁴ is cyclopropyl, cyclobutyl, cyclopentyl, or cyclohexyl. In some embodiments, R⁴ is cyclopropyl. In some embodiments, R⁴ is cyclobutyl. In some embodiments, R⁴ is cyclopentyl. In some embodiments, R⁴ is N(R⁵)R⁶. In some embodiments, R⁴ is NHR⁶. In some embodiments, R⁴ is NHR⁶, wherein R^{b} is optionally substituted 6-10 membered aryl. In some embodiments, R⁴ is NH (optionally substituted phenyl). In some embodiments, R⁴ is NH(phenyl). In some embodiments, R⁴ is an optionally substituted C₁-C₈ haloalkyl. In some embodiments, R⁴ is CHF₂.

In some such embodiments, R⁴ is hydrogen.

In some such embodiments, R⁴ is hydrogen; and R³ is N(R⁵)R⁶, optionally substituted 3-10 membered carbocyclyl, optionally substituted 3-10 membered heterocyclyl, optionally substituted 6-10 membered aryl, or optionally substituted 5-10 membered heteroaryl.

In some such embodiments, R⁴ is hydrogen; and R³ is optionally substituted phenyl, or optionally substituted 5-10 membered heteroaryl. In some such embodiments, R⁴ is hydrogen; and R³ is optionally substituted phenyl, or optionally substituted pyridinyl. In some such embodiments, R⁴ is hydrogen; and R³ is optionally substituted quinoxalinyl, naphthyridinyl, pyrazolopyridinyl, imidazopyridinyl, and pyrrolopyridinyl. In some such embodiments, R⁴ is hydrogen; and R³ is optionally substituted quinoxalinyl. In some such embodiments, R⁴ is hydrogen; and R³ is optionally substituted naphthyridinyl. In some such embodiments, R⁴ is hydrogen; and R³ is optionally substituted pyrazolopyridinyl. In some such embodiments, R⁴ is hydrogen; and R³ is optionally substituted imidazopyridinyl. In some such embodiments, R⁴ is hydrogen; and R³ is optionally substituted pyrrolopyridinyl.

In some embodiments of Formula (A) or Formula (I), each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl.

In some embodiments of Formula (A) or Formula (I), R⁵ and R⁶ together with the atom(s) to which they are attached optionally form an optionally substituted 4-7 membered heterocyclyl or optionally substituted 5-6 membered heteroaryl.

In one embodiment, the compound of Formula (I) has the structure of Formula (III):

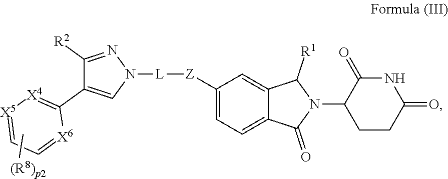

or a pharmaceutically acceptable salt thereof, wherein:
R¹, R², L and Z are defined as in Formula (I);
X⁴, X⁵ and X⁶ are each independently CR⁸ or N;
each R⁸ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
two R⁸ on adjacent carbon atoms are taken together to form an optionally substituted partially unsaturated 3-7 membered carbocyclyl, optionally substituted partially unsaturated 4-7 membered heterocyclyl, optionally substituted 6 membered aryl, or optionally substituted 5-6 membered heteroaryl; and
p₂ is 0, 1, 2, 3, 4, or 5.

In some embodiments of Formula (III), X⁴, X⁵ and X⁶ are each independently CR⁸. In some embodiments, X⁴, X⁵ and X⁶ are each independently N.

In some embodiments of Formula (III), X⁴ is N; and X⁵ and X⁶ are each independently CR⁸.

In some embodiments of Formula (III), X⁵ is N; and X⁴ and X⁶ are each independently CR⁸.

In some embodiments of Formula (III), X⁶ is N; and X⁴ and X⁵ are each independently CR⁸.

In some embodiments of Formula (III), X⁴ is CR⁸; and X⁵ and X⁶ are each independently N.

In some embodiments of Formula (III), X⁵ is CR⁸; and X⁴ and X⁶ are each independently N.

In some embodiments of Formula (III), X⁶ is CR⁸; and X⁴ and X⁵ are each independently N.

In some embodiments of Formula (III), p₂ is 0. In some embodiments, p₂ is 1. In some embodiments, p₂ is 2. In some embodiments, p₂ is 3. In some embodiments, p₂ is 4. In some embodiments, p₂ is 5.

In some embodiments of Formula (III), each R⁸ is independently hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some embodiments, each R⁸ is independently hydrogen, halogen, optionally substituted C₁-C₈ alkyl. In some embodiments, each R⁸ is independently hydrogen, halogen, or C₁-C₄ alkyl. In some embodiments, each R⁸ is independently methyl, ethyl, n-propyl, iso-propyl, or tert-butyl. In some embodiments, each R⁸ is independently hydrogen, halogen, methyl, ethyl, n-propyl, iso-propyl, or tert-butyl. In some embodiments, each R⁸ is independently an optionally substituted 3-10 membered carbocyclyl or optionally substituted 3-10 membered heterocyclyl. In some embodiments, each R⁸ is independently optionally substituted cyclopropyl, optionally substituted cyclobutyl, optionally substituted cyclopentyl, or optionally substituted cyclohexyl. In some embodiments, each R⁸ is independently cyclopropyl, cyclobutyl, cyclopentyl, or cyclohexyl. In some embodiments, each R⁸ is independently oxetane, tetrahydrofuran, tetrahydro-2H-pyran, pyrrolidine, piperidine, morpholine, or piperazine. In some embodiments, each R⁸ is independently hydrogen, Cl, F, Br, CN, NO₂, OH, OCH₃, methyl, ethyl, or iso-propyl. In some embodiments, each R⁸ is independently OR⁵, N(R⁵)R⁶, optionally substituted C₁-C₈ haloalkyl. In some embodiments, each R⁸ is independently cyclopropoxy, amino, methylamino, dimethylamino, CHF₂, and CF₃.

In some such embodiments, each R⁸ is independently halogen. In some such embodiments, each R⁸ is independently F. In some such embodiments, each R⁸ is independently Cl.

In some such embodiments, each R⁸ is independently optionally substituted C₁-C₈ alkyl. In some such embodiments, each R⁸ is independently optionally substituted C₁-C₃ alkyl. In some such embodiments, each R⁸ is independently C₁-C₃ haloalkyl. In some such embodiments, each R⁸ is independently CH₃. In some such embodiments, each R⁸ is independently CHF₂. In some such embodiments, each R⁸ is independently CF₃.

In some such embodiments, each R⁸ is independently optionally substituted 3-10 membered carbocyclyl. In some embodiments, each R⁸ is independently optionally substituted cyclopropyl, optionally substituted cyclobutyl, optionally substituted cyclopentyl, or optionally substituted cyclohexyl. In some embodiments, each R⁸ is independently optionally substituted cyclobutyl. In some such embodiments, each R⁸ is independently

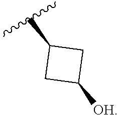 [SMILES: *[C@H]1C[C@@H](O)C1]

In some such embodiments, each R⁸ is independently optionally substituted 3-10 membered heterocyclyl. In some such embodiments, each R⁸ is independently optionally substituted 3-10 membered heterocyclyl comprising 1 or 2 nitrogens. In some such embodiments, each R⁸ is independently optionally substituted 3-10 membered heterocyclyl comprising 1 nitrogen. In some such embodiments, each R⁸ is independently optionally substituted 4, 5, or 6 membered heterocyclyl. In some such embodiments, each R⁸ is independently

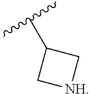 [SMILES: *C1CNC1]

In some such embodiments, each R⁸ is independently

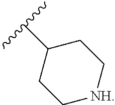 [SMILES: *C1CCNCC1]

In some such embodiments, each R⁸ is independently OR⁵. In some such embodiments, R⁵ is hydrogen, optionally substituted C₁-C₈ alkyl, or optionally substituted 3-10 membered carbocyclyl. In some such embodiments, R⁵ is optionally substituted 3-10 membered carbocyclyl. In some such embodiments, R⁵ is optionally substituted cyclopropyl, optionally substituted cyclobutyl, optionally substituted cyclopentyl, or optionally substituted cyclohexyl. In some such embodiments, each R⁸ is independently

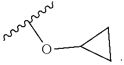 [SMILES: *OC1CC1]

In some such embodiments, each R⁸ is independently N(R⁵)R⁶. In some such embodiments, each R⁸ is independently NH₂. In some such embodiments, each R⁸ is independently —NHCH₃. In some such embodiments, each R⁸ is independently —N(CH₃)₂. In some such embodiments, R⁵ and R⁶ together with the Nitrogen to which they are attached form an optionally substituted 4-7 membered heterocyclyl. In some such embodiments, R⁵ and R⁶ together with the Nitrogen to which they are attached form an optionally substituted 4-7 membered heterocyclyl comprising 1-3 hetero atoms. In some such embodiments, R⁵ and R⁶ together with the Nitrogen to which they are attached form an optionally substituted 4-7 membered heterocyclyl comprising 1 nitrogen. In some such embodiments, R⁵ and R⁶ together with the Nitrogen to which they are attached form an optionally substituted 4-7 membered heterocyclyl comprising 2 nitrogens. In some such embodiments, R⁵ and R⁶ together with the Nitrogen to which they are attached form an optionally substituted 4, 5, or 6 membered heterocyclyl. In some such embodiments, each R⁸ is independently

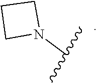 [SMILES: *N1CCC1]

In some such embodiments, each R⁸ is independently

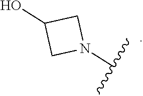 [SMILES: *N1CC(O)C1]

In some such embodiments, each R⁸ is independently

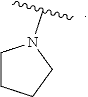 [SMILES: *N1CCCC1]

In some such embodiments, each R⁸ is independently

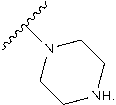 [SMILES: *N1CCNCC1]

In some such embodiments, two R⁸ on adjacent carbon atoms are taken together to form an optionally substituted partially unsaturated 3-7 membered carbocyclyl, optionally substituted partially unsaturated 4-7 membered heterocyclyl, optionally substituted phenyl, or optionally substituted 5-6 membered heteroaryl. In some embodiments, two R⁸ on adjacent carbon atoms are taken together to form an optionally substituted phenyl.

In some such embodiments, two R⁸ on adjacent carbon atoms are taken together to form an optionally substituted partially unsaturated 3-7 membered carbocyclyl. In some such embodiments, two R⁸ on adjacent carbon atoms are taken together to form an optionally substituted 5 membered carbocyclyl. In some such embodiments, two R⁸ on adjacent carbon atoms are taken together to form a 5 membered carbocyclyl substituted with a hydroxyl. In some such embodiments, two R⁸ on adjacent carbon atoms are taken together to form

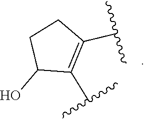 [SMILES: *C1=C(*)C(O)CC1]

In some such embodiments, two R⁸ on adjacent carbon atoms are taken together to form an optionally substituted partially unsaturated 4-7 membered heterocyclyl. In some such embodiments, two R⁸ on adjacent carbon atoms are taken together to form an optionally substituted 6 membered heterocyclyl. In some such embodiments, two R⁸ on adjacent carbon atoms are taken together to form an optionally substituted 6 membered heterocyclyl comprising 1 or 2 nitrogens. In some such embodiments, two R⁸ on adjacent carbon atoms are taken together to form an optionally substituted 6 membered heterocyclyl comprising 1 nitrogen. In some such embodiments, two R⁸ on adjacent carbon atoms are taken together to form optionally substituted

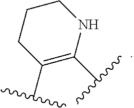 [SMILES: *C1=C(*)NCCC1]

In some such embodiments, two R⁸ on adjacent carbon atoms are taken together to form

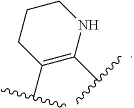 [SMILES: *C1=C(*)NCCC1]

In some embodiments, two R⁸ on adjacent carbon atoms are taken together to form a phenyl. In some embodiments, two R⁸ on adjacent carbon atoms are taken together to form a substituted phenyl. In some embodiments, two R⁸ on adjacent carbon atoms are taken together to form a phenyl substituted with a

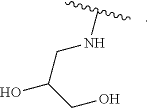 [SMILES: *NCC(O)CO]

In some embodiments, two R⁸ on adjacent carbon atoms are taken together to form a phenyl substituted with a

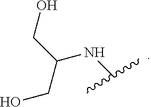 [SMILES: *NC(CO)CO]

In some embodiments, two R⁸ on adjacent carbon atoms are taken together to form a phenyl substituted with a

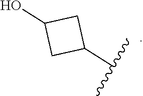 [SMILES: *C1CC(O)C1]

In some embodiments, two R⁸ on adjacent carbon atoms are taken together to form a phenyl substituted with a

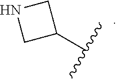 [SMILES: *C1CNC1]

In some embodiments, two R⁸ on adjacent carbon atoms are taken together to form a phenyl substituted with a

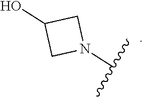 [SMILES: *N1CC(O)C1]

In some embodiments, two R⁸ on adjacent carbon atoms are taken together to form a phenyl substituted with a

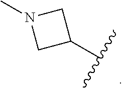 [SMILES: *C1CN(C)C1]

In some embodiments, two R⁸ on adjacent carbon atoms are taken together to form a phenyl substituted with a

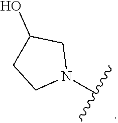 [SMILES: *N1CCC(O)C1]

In some embodiments, two R⁸ on adjacent carbon atoms are taken together to form a phenyl substituted with

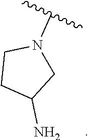 [SMILES: *N1CCC(N)C1]

In some embodiments, two R⁸ on adjacent carbon atoms are taken together to form

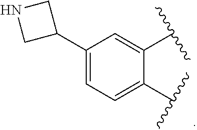 [SMILES: *c1ccc(C2CNC2)cc1*]

In some embodiments, two R⁸ on adjacent carbon atoms are taken together to form

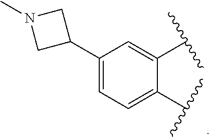 [SMILES: *c1ccc(C2CN(C)C2)cc1*]

In some embodiments, two R⁸ on adjacent carbon atoms are taken together to form

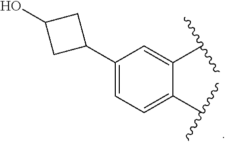 [SMILES: *c1ccc(C2CC(O)C2)cc1*]

In some embodiments, two R⁸ on adjacent carbon atoms are taken together to form

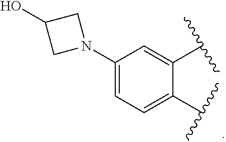 [SMILES: *c1ccc(N2CC(O)C2)cc1*]

In some embodiments, two R⁸ on adjacent carbon atoms are taken together to form

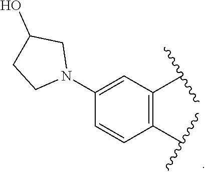 [SMILES: *c1ccc(N2CCC(O)C2)cc1*]

In some embodiments, two R⁸ on adjacent carbon atoms are taken together to form

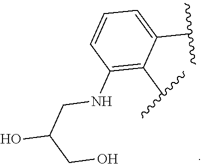 [SMILES: *c1cccc(NCC(O)CO)c1*]

In some embodiments, two R⁸ on adjacent carbon atoms are taken together to form

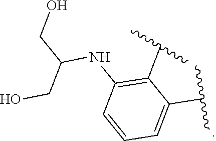 [SMILES: *c1cccc(NC(CO)CO)c1*]

In some embodiments, two R⁸ on adjacent carbon atoms are taken together to form

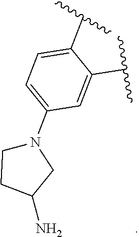 [SMILES: *c1ccc(N2CCC(N)C2)cc1*]

In some embodiments, two R⁸ on adjacent carbon atoms are taken together to form an optionally substituted 5-membered heteroaryl. In some embodiments, two R⁸ on adjacent carbon atoms are taken together to form an optionally substituted 5-membered heteroaryl comprising 1-3 nitrogen atoms. In some embodiments, two R⁸ on adjacent carbon atoms are taken together to form an optionally substituted 5-membered heteroaryl comprising 1 nitrogen atom. In some embodiments, two R⁸ on adjacent carbon atoms are taken together to form an optionally substituted 5-membered heteroaryl comprising 2 nitrogen atoms. In some embodiments, two R⁸ on adjacent carbon atoms are taken together to form an optionally substituted 5-membered heteroaryl comprising 3 nitrogen atoms. In some embodiments, two R⁸ on adjacent carbon atoms are taken together to form a substituted 5-membered heteroaryl, wherein the 5-membered heteroaryl is substituted with methyl. In some embodiments, two R⁸ on adjacent carbon atoms are taken together to form a substituted 5-membered heteroaryl, wherein the 5-membered heteroaryl is substituted with an iso-propyl. In some embodiments, two R⁸ on adjacent carbon atoms are taken together to form a substituted 5-membered heteroaryl, wherein the 5-membered heteroaryl is substituted with

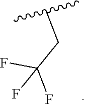 [SMILES: *CC(F)(F)F]

In some embodiments, two R⁸ on adjacent carbon atoms are taken together to form a substituted 5-membered heteroaryl, wherein the 5-membered heteroaryl is substituted with

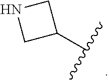 [SMILES: *C1CNC1]

In some embodiments, two R⁸ on adjacent carbon atoms are taken together to form a substituted 5-membered heteroaryl, wherein the 5-membered heteroaryl is substituted with

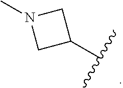 [SMILES: *C1CN(C)C1]

In some embodiments, two R⁸ on adjacent carbon atoms are taken together to form a substituted 5-membered heteroaryl, wherein the 5-membered heteroaryl is substituted with

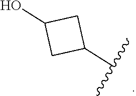 [SMILES: *C1CC(O)C1]

In some embodiments, two R⁸ on adjacent carbon atoms are taken together to form a substituted 5-membered heteroaryl, wherein the 5-membered heteroaryl is substituted with

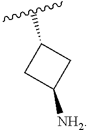 [SMILES: *[C@H]1C[C@H](N)C1]

In some embodiments, two R⁸ on adjacent carbon atoms are taken together to form a substituted 5-membered heteroaryl, wherein the 5-membered heteroaryl is substituted with

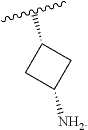 [SMILES: *[C@H]1C[C@@H](N)C1]

In some embodiments, two R⁸ on adjacent carbon atoms are taken together to form a substituted 5-membered heteroaryl, wherein the 5-membered heteroaryl is substituted with

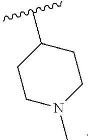 [SMILES: *C1CCN(C)CC1]

In some embodiments, two R⁸ on adjacent carbon atoms are taken together to form a substituted 5-membered heteroaryl, wherein the 5-membered heteroaryl is substituted with

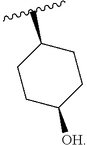 [SMILES: *[C@H]1CC[C@@H](O)CC1]

In some embodiments, two R⁸ on adjacent carbon atoms are taken together to form a substituted 5-membered heteroaryl, wherein the 5-membered heteroaryl is substituted with

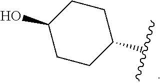 [SMILES: *[C@H]1CC[C@H](O)CC1]

In some embodiments, two R⁸ on adjacent carbon atoms are taken together to form

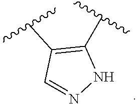 [SMILES: *c1cn[nH]c1*]

In some embodiments, two R⁸ on adjacent carbon atoms are taken together to form

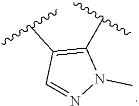 [SMILES: *c1cnn(C)c1*]

In some embodiments, two R⁸ on adjacent carbon atoms are taken together to form

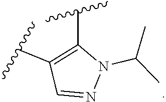 [SMILES: *c1cnn(C(C)C)c1*]

In some embodiments, two R⁸ on adjacent carbon atoms are taken together to form

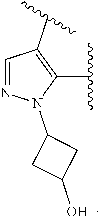 [SMILES: *c1cnn(C2CC(O)C2)c1*]

In some embodiments, two R⁸ on adjacent carbon atoms are taken together to form

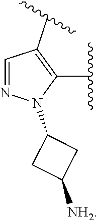 [SMILES: *c1cnn([C@H]2C[C@H](N)C2)c1*]

In some embodiments, two R⁸ on adjacent carbon atoms are taken together to form

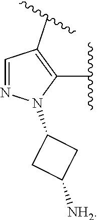 [SMILES: *c1cnn([C@H]2C[C@@H](N)C2)c1*]

In some embodiments, two R⁸ on adjacent carbon atoms are taken together to form

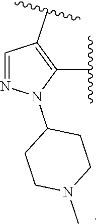 [SMILES: *c1cnn(C2CCN(C)CC2)c1*]

In some embodiments, two R⁸ on adjacent carbon atoms are taken together to form

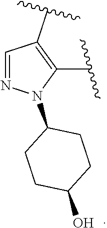 [SMILES: *c1cnn([C@H]2CC[C@@H](O)CC2)c1*]

In some embodiments, two R⁸ on adjacent carbon atoms are taken together to form

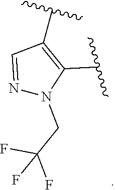 [SMILES: *c1cnn(CC(F)(F)F)c1*]

In some embodiments, two R⁸ on adjacent carbon atoms are taken together to form

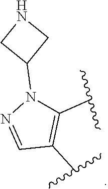 [SMILES: *c1cnn(C2CNC2)c1*]

In some embodiments, two R⁸ on adjacent carbon atoms are taken together to form

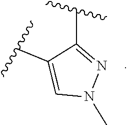 [SMILES: *c1cn(C)nc1*]

In some embodiments, two R⁸ on adjacent carbon atoms are taken together to form

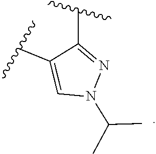 [SMILES: *c1cn(C(C)C)nc1*]

In some embodiments, two R⁸ on adjacent carbon atoms are taken together to form

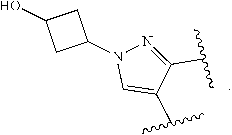 [SMILES: *c1cn(C2CC(O)C2)nc1*]

In some embodiments, two R⁸ on adjacent carbon atoms are taken together to form

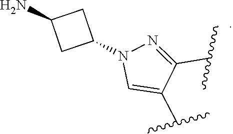 [SMILES: *c1cn([C@H]2C[C@H](N)C2)nc1*]

In some embodiments, two R⁸ on adjacent carbon atoms are taken together to form

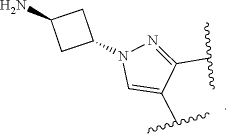 [SMILES: *c1cn([C@H]2C[C@H](N)C2)nc1*]

In some embodiments, two R⁸ on adjacent carbon atoms are taken together to form

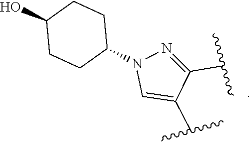 [SMILES: *c1cn([C@H]2CC[C@H](O)CC2)nc1*]

In some embodiments, two R⁸ on adjacent carbon atoms are taken together to form

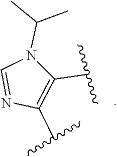 [SMILES: *c1ncn(C(C)C)c1*]

In some embodiments, two R⁸ on adjacent carbon atoms are taken together to form

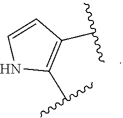 [SMILES: *c1cc[nH]c1*]

In some embodiments, two R⁸ on adjacent carbon atoms are taken together to form

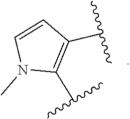 [SMILES: *c1ccn(C)c1*]

In some embodiments, two R⁸ on adjacent carbon atoms are taken together to form

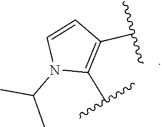 [SMILES: *c1ccn(C(C)C)c1*]

In some embodiments, two R⁸ on adjacent carbon atoms are taken together to form an optionally substituted 6-membered heteroaryl. In some embodiments, two R⁸ on adjacent carbon atoms are taken together to form an optionally substituted 6-membered heteroaryl comprising 1-3 nitrogen atoms. In some embodiments, two R⁸ on adjacent carbon atoms are taken together to form an optionally substituted 6-membered heteroaryl comprising 1 or 2 nitrogen atoms. In some embodiments, two R⁸ on adjacent carbon atoms are taken together to form an optionally substituted 6-membered heteroaryl comprising 1 nitrogen atom (i.e., pyridinyl). In some embodiments, two R⁸ on adjacent carbon atoms are taken together to form an optionally substituted

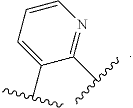 [SMILES: *c1cccnc1*]

In some embodiments, the compound of Formula (I) has the structure of Formula (IV):

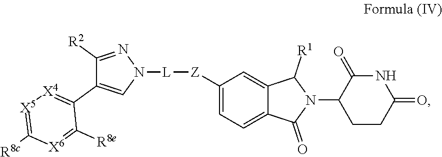

or a pharmaceutically acceptable salt thereof, wherein:
R¹, R², L and Z are defined as in Formula (I);
X⁴ is CR^{8a} or N;
X⁵ is CR^{8b} or N;
X⁶ is CR^{8d} or N;
R^{8a}, R^{8b}, R^{8c}, R^{8d}, and R^{8e} are each independently hydrogen, halogen, CN, NO₂, OR⁵, SR⁵, N(R⁵)R⁶, C(O)R⁵, C(O)OR⁵, C(O)N(R)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
R^{8a} and R^{8b}, R^{8b} and R^{8c}, R^{8c} and R^{8d}, or R^{8d} and R^{8e} are taken together with the atoms to which they are attached to form an optionally substituted 3-7 membered carbocyclyl, optionally substituted 4-7 membered heterocyclyl, optionally substituted phenyl, or optionally substituted 5-6 membered heteroaryl.

In some embodiments of Formula (IV), X⁴ is CR^{8a}, X⁵ is CR^{8b}, and X⁶ is CR^{8d}. In some embodiments of Formula (IV), X⁴ is N, X⁵ is CR^{8b}, and X⁶ is CR^{8d}. In some embodiments of Formula (IV), X⁵ is N, X⁴ is CR^{8a}, and X⁶ is CR^{8d}. In some such embodiments of the foregoing, R^{8a}, R^{8b}, R^{8c}, R^{8d}, and R^{8e} are each independently hydrogen, halogen, or optionally substituted C₁-C₈ alkyl. In some such embodiments, R^{8a}, R^{8b}, and R^{8c} are each independently hydrogen, halogen, or C₁-C₄ alkyl. In some such embodiments, R^{8d} and R^{8c} are hydrogen. In some embodiments of Formula (IV), X⁴ is N, X⁵ is CR^{8b}, and X⁶ is CR^{8d}, and R^{8b}, R^{8c}, R^{8d}, and R^{8e} are each independently hydrogen, halogen, or optionally substituted C₁-C₈ alkyl. In some such embodiments, R^{8b} and R^{8c} are each independently hydrogen, halogen, or C₁-C₄ alkyl, and R^{8d} and R^{8e} are hydrogen.

In some embodiments of Formula (IV), R^{8a} and R^{8b} are taken together with the atoms to which they are attached to form an optionally substituted phenyl. In some embodiments, R^{8a} and R^{8b} are taken together with the atoms to which they are attached to form an optionally substituted 5-membered heteroaryl. In some embodiments, R^{8a} and R^{8b} are taken together with the atoms to which they are attached to form an optionally substituted 6-membered heteroaryl comprising 1-3 nitrogen atoms.

In some embodiments of Formula (IV), R^{8b} and R^{8c} are taken together with the atoms to which they are attached to form an optionally substituted phenyl. In some embodiments, R^{8b} and R^{8c} are taken together with the atoms to which they are attached to form an optionally substituted 5-membered heteroaryl. In some such embodiments, R^{8b} and R^{8c} are taken together with the atoms to which they are attached to form an optionally substituted 6-membered heteroaryl comprising 1-3 nitrogen atoms.

In some embodiments of Formula (IV), R^{8c} and R^{8d} are taken together with the atoms to which they are attached to form an optionally substituted phenyl or optionally substituted 5-6 membered heteroaryl. In some embodiments, R^{8c} and R^{8d} are taken together with the atoms to which they are attached to form an optionally substituted phenyl. In some embodiments, R^{8c} and R^{8d} are taken together with the atoms to which they are attached to form an optionally substituted 5-membered heteroaryl. In some embodiments, R^{8c} and R^{8d} are taken together with the atoms to which they are attached to form an optionally substituted 6-membered heteroaryl comprising 1-3 nitrogen atoms.

In some embodiments of Formula (IV), R^{8d} and R^{8e} are taken together with the atoms to which they are attached to form an optionally substituted phenyl or optionally substituted 5-6 membered heteroaryl. In some embodiments, R^{8d} and R^{8c} are taken together with the atoms to which they are attached to form an optionally substituted phenyl. In some embodiments, R^{8d} and R^{8e} are taken together with the atoms to which they are attached to form an optionally substituted 5-membered heteroaryl. In some embodiments, R^{8d} and R^{8e} are taken together with the atoms to which they are attached to form an optionally substituted 6-membered heteroaryl comprising 1-3 nitrogen atoms.

In some embodiments of Formula (IV), X⁴ is N; and R^{8b} and R^{8c} are taken together with the atoms to which they are attached to form an optionally substituted phenyl or optionally substituted 5-6 membered heteroaryl.

Embodiments described herein for Formula (IV) are also applicable for Formula (IV-A), (IV-B), (IV-C) or (IV-D), to the extent that such embodiments are not inconsistent.

In some embodiments, the compound of Formula (IV) has the structure of Formula (IV-A):

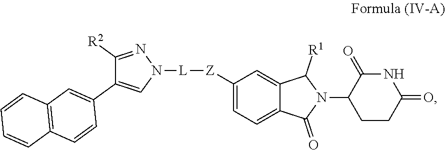

or a pharmaceutically acceptable salt thereof, wherein:
R¹, R², L and Z are defined as in Formula (T);
X⁴ is CR^{8a} or N;
X⁶ is CR^{8d} or N;
each R⁹ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; and
q₁ is 0, 1, 2, or 3.

In some such embodiments of Formula (IV-A), each R⁹ is independently hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some embodiments, each R⁹ is independently hydrogen, halogen, optionally substituted C₁-C₈ alkyl. In some embodiments, each R⁹ is independently methyl, ethyl, n-propyl, iso-propyl, or tert-butyl. In some embodiments, each R⁹ is independently an optionally substituted 3-10 membered carbocyclyl or optionally substituted 3-10 membered heterocyclyl. In some embodiments, each R⁹ is independently cyclopropyl, cyclobutyl, cyclopentyl, or cyclohexyl. In some embodiments, each R⁹ is independently optionally substituted cyclopropyl, optionally substituted cyclobutyl, optionally substituted cyclopentyl, or optionally substituted cyclohexyl. In some embodiments, each R⁹ is independently oxetane, tetrahydrofuran, tetrahydro-2H-pyran, pyrrolidine, piperidine, morpholine, or piperazine. In some embodiments, each R⁹ is independently hydrogen, Cl, F, Br, CN, NO₂, OH, OCH₃, methyl, ethyl, or iso-propyl. In some embodiments, each R⁹ is independently optionally substituted C₁-C₈ alkylamino. In some embodiments, each R⁹ is independently NH(CH₂OH)₂ or NHCH₂CH(OH)CH₂OH.

In some embodiments of Formula (IV-A), q₁ is 1, 2, or 3. In some embodiments, q₁ is 1 or 2. In some embodiments, q₁ is 0. In some embodiments, q₁ is 1. In some embodiments, q₁ is 2. In some embodiments, q₁ is 3.

In some embodiments, X⁴ is N and X⁶ is N. In some embodiments, X⁴ is N and X⁶ is CR^{8a}. In some embodiments, X⁴ is CR^{8a} and X⁶ is N. In some embodiments, X⁴ is CR^{8a} and X⁶ is CR^{8d}.

In some embodiments, the compound of Formula (IV) has the structure of Formula (IV-B):

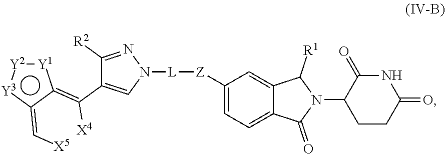

or a pharmaceutically acceptable salt thereof, wherein:
R¹, R², L and Z are defined as in Formula (I);
X⁴ is CR^{8a} or N;
X⁵ is CR^{8b} or N;
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

In some such embodiments of Formula (IV-B), X⁴ is N and X⁵ is CR^{8b}. In some embodiments, X⁵ is N and X⁴ is CR^{8a}. In some embodiments, X⁴ is N and X⁵ is N. In some embodiments, X⁴ is CR^{8a} and X⁵ is CR^{8b}.

In some embodiments, the compound of Formula (IV) has the structure of Formula (IV-C):

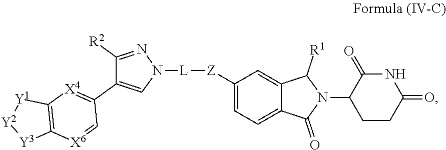

or a pharmaceutically acceptable salt thereof, wherein:
R¹, R², L and Z are defined as in Formula (I);
X⁴ is CR^{8a} or N;
X⁶ is CR^{8d} or N;
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

In some such embodiments of Formula (IV-C), X⁴ is N; and X⁶ is CR^{8d}. In some embodiments, X⁴ is CR^{8a}; and X⁶ is N. In some embodiments, X⁴ is N; and X⁶ is N.

In some embodiments, the compound of Formula (IV) has the structure of Formula (IV-D):

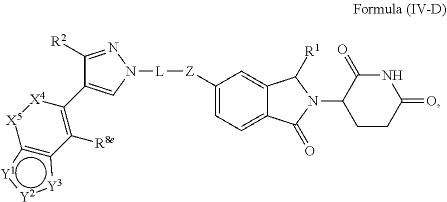

or a pharmaceutically acceptable salt thereof, wherein:
R¹, R², L and Z are defined as in Formula (I);
X⁴ is CR^{8a} or N;
X⁵ is CR^{8b} or N;
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

In some such embodiments of Formula (IV-D), X⁴ is N; and X⁵ is CR^{8b}. In some embodiments, X⁴ is CR^{8a}; and X⁵ is N. In some embodiments, X⁴ is N; and X⁵ is N.

In some embodiments of any of Formulae (IV), (IV-A), (IV-B), (IV-C) or (IV-D), R^{8a}, R^{8b}, R^{8c}, R^{8d}, and R^{8e} are each independently hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some such embodiments, R^{8a}, R^{8b}, R^{8c}, R^{8d}, and R^{8e} are each independently hydrogen, halogen, or optionally substituted C₁-C₈ alkyl. In some such embodiments, R^{8a}, R^{8b}, R^{8c}, R^{8d}, and R^{8e} are each independently hydrogen, halogen, or C₁-C₄ alkyl. In some embodiments, R^{8a}, R^{8b}, R^{8c}, R^{8d}, and R^{8e} are each independently hydrogen, halogen, methyl, ethyl, n-propyl, iso-propyl, or tert-butyl. In some embodiments, R^{8a}, R^{8b}, R^{8c}, R^{8d}, and R^{8e} are each independently methyl, ethyl, n-propyl, iso-propyl, or tert-butyl. In some embodiments, R^{8a}, R^{8b}, R^{8c}, R^{8d}, and R^{8e} are each independently optionally substituted 3-10 membered carbocyclyl, or optionally substituted 3-10 membered heterocyclyl. In some embodiments, R^{8a}, R^{8b}, R^{8c}, R^{8d}, and R^{8e} are each independently cyclopropyl, cyclobutyl, cyclopentyl, or cyclohexyl. In some embodiments, R^{8a}, R^{8b}, R^{8c}, R^{8d}, and R^{8e} are each independently hydrogen, Cl, F, Br, CN, NO₂, OH, OCH₃, methyl, ethyl, or iso-propyl. In some embodiments, Ra, R^{8b}, R^{8c}, R^{8d}, and R^{e} are each independently hydrogen or F.

In some embodiments of Formula (IV-B), (IV-C) or (IV-D), Y¹ and Y³ are each independently NR^{10a} or N; and Y² is CR¹⁰.

In some embodiments of Formula (IV-B), (IV-C) or (IV-D), Y² and Y³ are each independently NR^{10a} or N; and Y¹ is CR¹⁰.

In some embodiments of Formula (IV-B), (IV-C) or (IV-D), Y¹ and Y² are each independently NR^{10a} or N; and Y³ is CR¹⁰.

In some embodiments of Formula (IV-B), (IV-C) or (IV-D), each R¹⁰ is independently hydrogen, halogen, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl. In some embodiments, each R¹⁰ is independently hydrogen, halogen, optionally substituted C₁-C₈ alkyl or an optionally substituted 3-10 membered carbocyclyl. In some embodiments, each R¹⁰ is independently methyl, ethyl, n-propyl, iso-propyl, or tert-butyl. In some embodiments, each R¹⁰ is independently cyclopropyl, cyclobutyl, cyclopentyl, or cyclohexyl. In some embodiments, each R¹⁰ is independently hydrogen. In some embodiments, each R¹⁰ is independently halogen. In some embodiments, each R¹⁰ is independently F, Cl or Br. In some embodiments, each R¹⁰ is independently F.

In some embodiments of Formula (IV-B), (IV-C) or (IV-D), each R^{10a} is independently hydrogen, or optionally substituted C₁-C₈ alkyl, optionally substituted C₁-C₈ haloalkyl. In some embodiments, In some embodiments, each R^{10a} is independently methyl, ethyl, n-propyl, iso-propyl, or tert-butyl. In some embodiments, each R^{10a} is independently hydrogen. In some embodiments, each R^{10a} is independently optionally substituted C₁-C₈ haloalkyl. In some embodiments, each R^{10a} is independently CF₃, CHF₂, or CH₂CF₃. In some embodiments, each R^{10a} is independently optionally substituted 3-6 membered carbocyclyl. In some embodiments, each R^{10a} is independently optionally substituted cyclopropyl, optionally substituted cyclobutyl, optionally substituted cyclopentyl, or optionally substituted cyclohexyl. In some embodiments, each R^{10a} is independently optionally substituted 3-6 membered heterocyclyl. In some embodiments, each R^{10a} is independently optionally substituted oxetanyl, optionally substituted tetrahydrofuranyl, optionally substituted tetrahydro-2H-pyranyl, optionally substituted azetidinyl, optionally substituted pyrrolidinyl, optionally substituted piperidinyl, optionally substituted morpholinyl, or optionally substituted piperazinyl.

In some embodiments of any of the Formulae described herein, R² is hydrogen, halogen, CN, NO₂, OR⁵, SR⁵, N(R⁵)R⁶, C(O)R⁵, optionally substituted C₁-C₈ alkyl, optionally substituted C₁-C₈ haloalkyl, optionally substituted C₁-C₈ alkoxy, optionally substituted C₁-C₈ alkylamino, optionally substituted 3-10 membered carbocyclyl, or optionally substituted 3-10 membered heterocyclyl. In some embodiments, R² is halogen, CN, OR⁵, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl.

In some such embodiments of any of the Formulae described herein, each R² is hydrogen, halogen, optionally substituted C₁-C₈ alkyl, or optionally substituted 3-10 membered carbocyclyl. In some embodiments, R² is methyl, ethyl, n-propyl, iso-propyl, or tert-butyl. In some embodiments, R² is methyl or ethyl. In some embodiments, R² is methyl. In some embodiments, R² is iso-propyl. In some embodiments, R² is optionally substituted 3-10 membered heterocyclyl. In some embodiments, R² is oxetane, tetrahydrofuran, tetrahydro-2H-pyran, pyrrolidine, piperidine, morpholine, or piperazine. In some embodiments, R² is an optionally substituted 3-10 membered carbocyclyl. In some embodiments, R² is cyclopropyl, cyclobutyl, cyclopentyl, or cyclohexyl. In some embodiments, R² is cyclopropyl. In some embodiments, R² is cyclobutyl. In some embodiments, R² is cyclopentyl. In some embodiments, R² is optionally substituted C₁-C₈ haloalkyl. In some embodiments, R² is CF₃, CHF₂, or CH₂CF₃. In some embodiments, R² is CHF₂.

In some embodiments of any of the Formulae described herein, Z is absent or —O—. In some embodiments, Z is absent. In some embodiments, Z is —O—. In some embodiments, Z is —NR^{1a}—. In some embodiments, Z is —NR^{1a}—Z, wherein R^{1a} is hydrogen or C₁-C₄ alkyl. In some embodiments, Z is —NR^{1a}— wherein R^{1a} is hydrogen. In some embodiments, Z is —NH—.

In some embodiments of any of the Formulae described herein, L is an optionally substituted C₁-C₁₀ alkylene or optionally substituted C₁-C₁₀ heteroalkylene. In some embodiments, L is an optionally substituted C₁-C₁₀ alkylene. In some embodiments, L is an optionally substituted C₁-C₁₀ heteroalkylene, wherein the heteroatom is N or O. In some embodiments, the heteroalkylene is polyethylene glycol (PEG).

In some embodiments of any of the Formulae described herein, L is

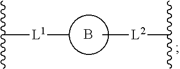

wherein
Ring B is an optionally substituted 3-7 membered carbocyclyl or optionally substituted 4-7 membered heterocyclyl;
L¹ is absent, or an optionally substituted C₁-C₁₀ alkylene or optionally substituted C₁-C₁₀ heteroalkylene; and
L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene.

In some such embodiments, L is

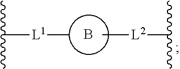

wherein
Ring B is an optionally substituted 3-7 membered carbocyclyl;
L¹ is absent or an optionally substituted C₁-C₁₀ alkylene; and
L² is absent or an optionally substituted C₁-C₁₀ alkylene.

In some embodiments, L is

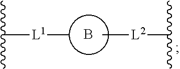

wherein L¹ is absent; and L² is C₁-C₁₀ alkylene.

In some such embodiments, L² is C₁-C₄ alkylene, preferably L² is C₁-C₂ alkylene.

In some embodiments, L is

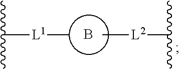

wherein L² is absent; and L¹ is C₁-C₁₀ alkylene. In some such embodiments, L¹ is C₁-C₄ alkylene, preferably L¹ is C₁-C₂ alkylene.

In some embodiments, Ring B is an optionally substituted 3-7 membered carbocyclyl. In some embodiments, Ring B is an optionally substituted cyclopropyl, optionally substituted cyclobutyl, optionally substituted cyclopentyl, optionally substituted cyclohexyl, optionally substituted optionally substituted spiro[3.3]heptyl, optionally substituted spiro[4.4]nonyl, or optionally substituted spiro[3.4]octanyl ring. In some embodiments, Ring B is an optionally substituted cyclopropyl, optionally substituted cyclobutyl, optionally substituted cyclopentyl, or optionally substituted cyclohexyl. In some embodiments, Ring B is an optionally substituted cyclopropyl. In some embodiments, Ring B is an optionally substituted cyclobutyl. In some embodiments, Ring B is an optionally substituted cyclopentyl. In some embodiments, Ring B is an optionally substituted cyclohexyl. In some embodiments, Ring B is cyclopropyl, cyclobutyl, cyclopentyl or cyclohexyl. In some embodiments, Ring B is an optionally substituted spiro[3.3]heptyl, optionally substituted spiro[4.4]nonyl or optionally substituted spiro[3.4]octanyl ring. In some embodiments, Ring B is an optionally substituted spiro[3.3]heptyl or optionally substituted spiro[4.4]nonyl. In some embodiments, Ring B is an optionally substituted spiro[3.3]heptyl ring. In some embodiments, Ring B is an optionally substituted spiro[4.4]nonyl ring.

In some embodiments of any of the formulae described herein, L¹ is absent. In some embodiments, L¹ is an optionally substituted C₁-C₁₀ alkylene. In some embodiments, L¹ is an optionally substituted C₁-C₄ alkylene. In some embodiments, L¹ is —CH₂—, —CH₂CH₂—, or —CH₂CH₂CH₂—. In some embodiments, L¹ is an optionally substituted C₁-C₁₀ heteroalkylene wherein the heteroatom is N or O. In some embodiments, L¹ is polyethylene glycol (PEG). In some embodiments, L¹ is —(CH₂CH₂O)ₙ—, wherein n is an integer from 1-20. In some embodiments, n is 1-15, 1-10, 1-8, 1-6, 1-4, or 1-2.

In some embodiments of any of the formulae described herein, L² is an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene. In some embodiments of any of the formulae described herein, L² is an optionally substituted C₁-C₄ alkylene, optionally substituted C₂-C₄ alkenylene, optionally substituted C₂-C₄ alkynylene, or optionally substituted C₁-C₄ heteroalkylene. In some embodiments, L² is absent. In some embodiments, L² is —CH₂—, —CH₂CH₂—, or —CH₂CH₂CH₂—. In some embodiments, L² is an optionally substituted C₁-C₁₀ heteroalkylene wherein the heteroatom is N or O. In some embodiments, L² is polyethylene glycol (PEG). In some such embodiments, L² is —(CH₂CH₂O)ₙ—, wherein n is an integer from 1-20. In some embodiments, n is 1-15, 1-10, 1-8, 1-6, 1-4, or 1-2.

In some embodiments of any of the Formulae described herein, R¹ is absent. When R¹ is absent, the moiety has the structure:

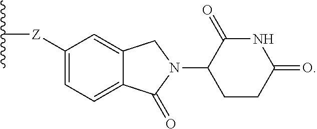

In some embodiments of any of the formulae described herein, R¹ is oxo (═O). When R¹ is oxo, the moiety has the structure:

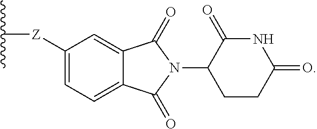

In some embodiments of Formula (II) or (II-A), p₁ is 1, 2, or 3. In some embodiments, p₁ is 1 or 2. In some embodiments, p₁ is 0. In some embodiments, p₁ is 1. In some embodiments, p₁ is 2. In some embodiments, p₁ is 3.

In some embodiments of Formula (III), p₂ is 1, 2, 3 or 4. In some embodiments, p₂ is 1 or 2. In some embodiments, p₂ is 0. In some embodiments, p₂ is 1. In some embodiments, p₂ is 2. In some embodiments, p₂ is 3. In some embodiments, p₂ is 4.

Some preferred embodiments of Formula (III) have any combination of one, two, three, four, five, or more than five of the following selections to the extent such embodiments are not incompatible:
R² is hydrogen, halogen, optionally substituted C₁-C₈ alkyl, or optionally substituted 3-10 membered carbocyclyl;
R² is an optionally substituted 3-10 membered carbocyclyl;
R² is cyclopropyl, cyclobutyl, cyclopentyl or cyclohexyl;
R² is cyclopropyl;
X⁴ is N, and X⁵ and X⁶ are each independently CR⁸;
X⁴ is N, X⁵ is N, and X⁶ is CR⁸;
p2 is 1 or 2;
p2 is 2;
each R⁸ is independently hydrogen, halogen, or optionally substituted C₁-C₈ alkyl;
each R⁸ is independently hydrogen, F, or C₁-C₄ alkyl;
each R⁸ is independently F or methyl;
L is

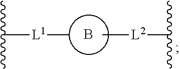

wherein:
   L¹ is absent; and L² is C₁-C₁₀ alkylene;
   L¹ is absent; and L² is C₁-C₄ alkylene; or
   L¹ is absent; and L² is C₁-C₂ alkylene; L is

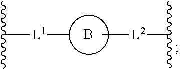

wherein:
   L² is absent; and L¹ is C₁-C₁₀ alkylene;
   L² is absent; and L¹ is C₁-C₄ alkylene; or
   L² is absent; and L¹ is C₁-C₂ alkylene;
Ring B is an optionally substituted 3-7 membered carbocyclyl;
Ring B is an optionally substituted cyclopropyl, optionally substituted cyclobutyl, optionally substituted cyclopentyl, or optionally substituted cyclohexyl;
Ring B is an optionally substituted cyclobutyl;
Ring B is an optionally substituted spiro[3.3]heptyl, optionally substituted spiro[4.4]nonyl or optionally substituted spiro[3.4]octanyl ring;
Z is —NR^{1a}—, wherein R^{1a} is hydrogen or C₁-C₄ alkyl;
Z is —NR^{1a}—, wherein R^{1a} is hydrogen;
Z is —NH—;
R¹ is absent;
R¹ is oxo.

Some preferred embodiments of Formula (IV) have any combination of one, two, three, four, five, or more than five of the following selections to the extent such embodiments are not incompatible:
R² is hydrogen, halogen, optionally substituted C₁-C₈ alkyl, or optionally substituted 3-10 membered carbocyclyl;
R² is an optionally substituted 3-10 membered carbocyclyl;
R² is cyclopropyl, cyclobutyl, cyclopentyl or cyclohexyl;
R² is cyclopropyl;
X⁴ is N, X⁵ is CR^{8b} and X⁶ is CR^{8d}
X⁴ is N, X⁵ is N, and X⁶ is CR^{8d};
X⁵ is N, X⁴ is CR^{8a}, and X⁶ is CR^{8d};
R^{8a}, R^{8b}, R^{8c}, R^{8d}, and R^{8e} are each independently hydrogen, halogen, or optionally substituted C₁-C₈ alkyl;
R^{8a}, R^{8b}, R^{8c}, R^{8d}, and R^{8e} are each independently hydrogen, halogen, or C₁-C₄ alkyl;
R^{8a}, R^{8b}, R^{8c}, R^{8d}, and R^{8e} are each independently hydrogen, F, or C₁-C₄ alkyl;
R^{8a} and R^{8b} are taken together with the atoms to which they are attached to form an optionally substituted 5-6 membered heteroaryl;
R^{8b} and R^{8c} are taken together with the atoms to which they are attached to form an optionally substituted phenyl;
R^{8b} and R^{8c} are taken together with the atoms to which they are attached to form an optionally substituted 5-6 membered heteroaryl;
R^{8c} and R^{8d} are taken together with the atoms to which they are attached to form an optionally substituted 5-6 membered heteroaryl;
L is

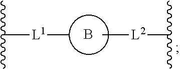

wherein:
   L¹ is absent; and L² is C₁-C₁₀ alkylene;
   L¹ is absent; and L² is C₁-C₄ alkylene; or
   L¹ is absent; and L² is C₁-C₂ alkylene;
L is

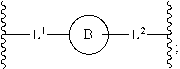

wherein:
   L² is absent; and L¹ is C₁-C₁₀ alkylene;
   L² is absent; and L¹ is C₁-C₄ alkylene; or
   L² is absent; and L¹ is C₁-C₂ alkylene;
Ring B is an optionally substituted 3-7 membered carbocyclyl;
Ring B is an optionally substituted cyclopropyl, optionally substituted cyclobutyl, optionally substituted cyclopentyl, or optionally substituted cyclohexyl;
Ring B is an optionally substituted cyclobutyl;
Ring B is an optionally substituted spiro[3.3]heptyl, optionally substituted spiro[4.4]nonyl or optionally substituted spiro[3.4]octanyl ring;
Z is —NR^{1a}—, wherein R^{1a} is hydrogen or C₁-C₄ alkyl;
Z is —NR^{1a}—, wherein R^{1a} is hydrogen;
Z is —NH—;
R¹ is absent;
R¹ is oxo.

Some preferred embodiments of Formula (IV-A), (IV-B), (IV-C) or (IV-D) have any combination of one, two, three, four, five, or more than five of the following selections to the extent such embodiments are not incompatible:
R² is hydrogen, halogen, optionally substituted C₁-C₈ alkyl, or optionally substituted 3-10 membered carbocyclyl;
R² is an optionally substituted 3-10 membered carbocyclyl;
R² is cyclopropyl, cyclobutyl, cyclopentyl or cyclohexyl;
R² is cyclopropyl;
Formula (IV-A) or (IV-C):
   X⁴ is N, and X⁶ is CR^{8d};
   X⁴ is N, and X⁶ is N;
Formula (IV-B) or (IV-D):
   X⁴ is N, and X⁵ is CR^{8b};
   X⁴ is N, and X⁵ is N;
Formula (IV-B), (IV-C) or (IV-D):
   Y¹ and Y³ are each independently NR^{10a} or N; and Y² is CR¹⁰;
   Y² and Y³ are each independently NR^{10a} or N; and Y¹ is CR¹⁰;
Y¹ and Y² are each independently NR^{10a} or N; and Y³ is CR¹⁰;
L is

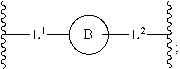

wherein:
   L¹ is absent; and L² is C₁-C₁₀ alkylene;
   L¹ is absent; and L² is C₁-C₄ alkylene; or
   L¹ is absent; and L² is C₁-C₂ alkylene;
L is

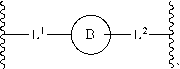

wherein:
   L² is absent; and L¹ is C₁-C₁₀ alkylene;
   L² is absent; and L¹ is C₁-C₄ alkylene; or
   L² is absent; and L¹ is C₁-C₂ alkylene;
Ring B is an optionally substituted 3-7 membered carbocyclyl;
Ring B is an optionally substituted cyclopropyl, optionally substituted cyclobutyl, optionally substituted cyclopentyl, or optionally substituted cyclohexyl;
Ring B is an optionally substituted cyclobutyl;
Ring B is an optionally substituted spiro[3.3]heptyl, optionally substituted spiro[4.4]nonyl or optionally substituted spiro[3.4]octanyl ring;
Z is —NR^{1a}—, wherein R^{1a} is hydrogen or C₁-C₄ alkyl;
Z is —NR^{1a}—, wherein R^{1a} is hydrogen;
Z is —NH—;
R¹ is absent;
R¹ is oxo.

Disclosed herein, are GSPT1 degraders. In some embodiments, the compounds disclosed herein or pharmaceutically acceptable salts thereof degrade GSPT1. In some embodiments, the compounds disclosed herein or pharmaceutically acceptable salts thereof reduce cellular GSPT1 protein levels. In some embodiments, the compounds bind cereblon. In some embodiments, the compounds modulates cereblon. In some embodiments, the compounds degrades GSPT1 as a downstream effect of cereblon binding and modulation. Some examples of GSTP1 degraders are shown in Table 1.

TABLE 1
Exemplary compounds of the disclosure.

| Cpd. | | |
|---|---|---|
| code | Structure | Chemical Name |
| GS- 640 | 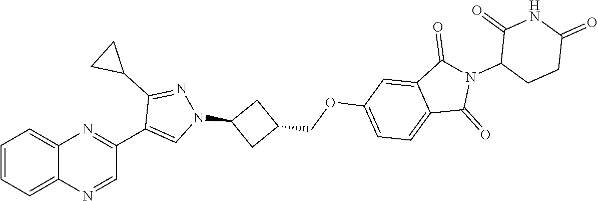 [SMILES: O=C1CCC(N2C(=O)c3ccc(OC[C@H]4C[C@H](n5cc(-c6cnc7ccccc7n6)c(C6CC6)n5)C4)cc3C2=O)C(=O)N1] | 5-((trans-3-(3-cyclopropyl-4- (quinoxalin-2-yl)-1//-pyrazol-1- yl)cyclobutyl)methoxy)-2-(2,6- dioxopiperidin-3-yl)isoindoline-1,3- dione |
| GS- 641 | 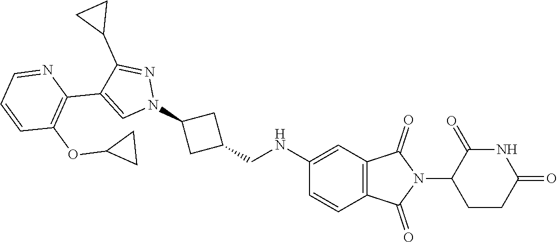 [SMILES: O=C1CCC(N2C(=O)c3ccc(NC[C@H]4C[C@H](n5cc(-c6ncccc6OC6CC6)c(C6CC6)n5)C4)cc3C2=O)C(=O)N1] | 5-(((trans-3-(4-(3-cyclopropoxypyridin- 2-yl)-3-cyclopropyl-1H-pyrazol-1- yl)cyclobutyl)methyl)amino)-2-(2,6- dioxopiperidin-3-yl)isoindoline-1,3- dione |
| GS- 642 | 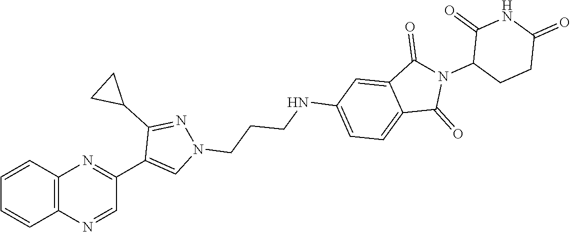 [SMILES: O=C1CCC(N2C(=O)c3ccc(NCCCn4cc(-c5cnc6ccccc6n5)c(C5CC5)n4)cc3C2=O)C(=O)N1] | 5-((3-(3-cyclopropyl-4-(quinoxalin-2- yl)-1H-pyrazol-1-yl)propyl)amino)-2- (2,6-dioxopiperidin-3-yl)isoindoline-1,3- dione |
| GS- 643 | 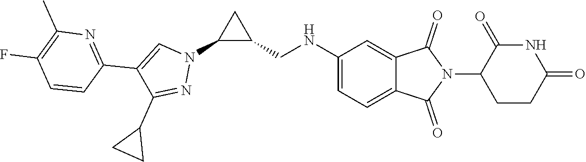 [SMILES: Cc1nc(-c2cn([C@H]3C[C@@H]3CNc3ccc4c(c3)C(=O)N(C3CCC(=O)NC3=O)C4=O)nc2C2CC2)ccc1F] | 5-(((trans-2-(3-cyclopropyl-4-(5-fluoro- 6-methylpyridin-2-yl)-1H-pyrazol-1- yl)cyclopropyl)methyl)amino)-2-(2,6- dioxopiperidin-3-yl)isoindoline-1,3- dione |
| GS- 644 | 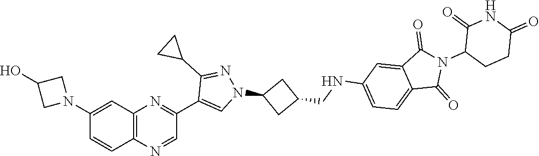 [SMILES: O=C1CCC(N2C(=O)c3ccc(NC[C@H]4C[C@H](n5cc(-c6cnc7ccc(N8CC(O)C8)cc7n6)c(C6CC6)n5)C4)cc3C2=O)C(=O)N1] | 5-(((trans-3-(3-cyclopropyl-4-(7-(3- hydroxyazetidin-1-yl)quinoxalin-2-yl)- 1H-pyrazol-1- yl)cyclobutyl)methyl)amino)-2-(2,6- dioxopiperidin-3-yl)isoindoline-1,3- dione |
| GS- 645 | 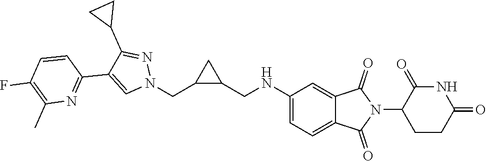 [SMILES: Cc1nc(-c2cn(CC3CC3CNc3ccc4c(c3)C(=O)N(C3CCC(=O)NC3=O)C4=O)nc2C2CC2)ccc1F] | 5-(((2-((3-cyclopropyl-4-(5-fluoro-6- methylpyridin-2-yl)-1H-pyrazol-1- yl)methyl)cyclopropyl)methyl)amino)-2- (2,6-dioxopiperidin-3-yl)isoindoline-1,3- dione |
| GS- 646 | 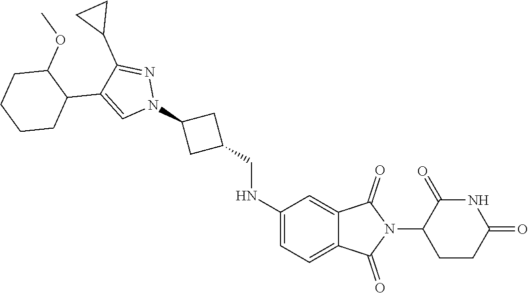 [SMILES: COC1CCCCC1c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(2- methoxycyclohexyl)-1H-pyrazol-1- yl)cyclobutyl)methyl)amino)-2-(2,6- dioxopiperidin-3-yl)isoindoline-1,3- dione |
| GS- 647 | 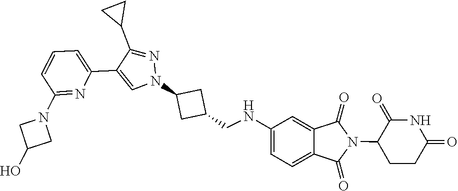 [SMILES: O=C1CCC(N2C(=O)c3ccc(NC[C@H]4C[C@H](n5cc(-c6cccc(N7CC(O)C7)n6)c(C6CC6)n5)C4)cc3C2=O)C(=O)N1] | 5-(((trans-3-(3-cyclopropyl-4-(6-(3- hydroxyazetidin-1-yl)pyridin-2-yl)-1H- pyrazol-1-yl)cyclobutyl)methyl)amino)- 2-(2,6-dioxopiperidin-3-yl)isoindoline- 1,3-dione |
| GS- 648 | 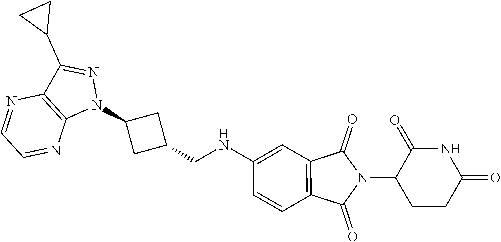 [SMILES: O=C1CCC(N2C(=O)c3ccc(NC[C@H]4C[C@H](n5nc(C6CC6)c6nccnc65)C4)cc3C2=O)C(=O)N1] | 5-(((trans-3-(3-cyclopropyl-1H- pyrazolo[3,4-b]pyrazin-1- yl)cyclobutyl)methyl)amino)-2-(2,6- dioxopiperidin-3-yl)isoindoline-1,3- dione |
| GS- 649 | 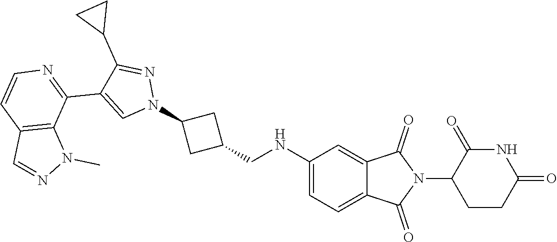 [SMILES: Cn1ncc2ccnc(-c3cn([C@H]4C[C@H](CNc5ccc6c(c5)C(=O)N(C5CCC(=O)NC5=O)C6=O)C4)nc3C3CC3)c21] | 5-(((trans-3-(3-cyclopropyl-4-(1-methyl- 1H-pyrazolo[3,4-c]pyridin-7-yl)-1H- pyrazol-1-yl)cyclobutyl)methyl)amino)- 2-(2,6-dioxopiperidin-3-yl)isoindoline- 1,3-dione |
| GS- 650 | 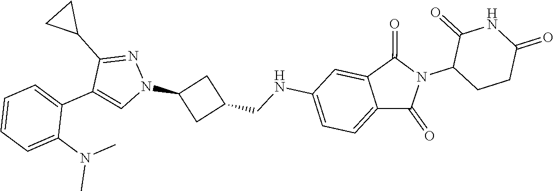 [SMILES: CN(C)c1ccccc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(2- (dimethylamino)phenyl)-1H-pyrazol-1- yl)cyclobutyl)methyl)amino)-2-(2,6- dioxopiperidin-3-yl)isoindoline-1,3- dione |
| GS- 651 | 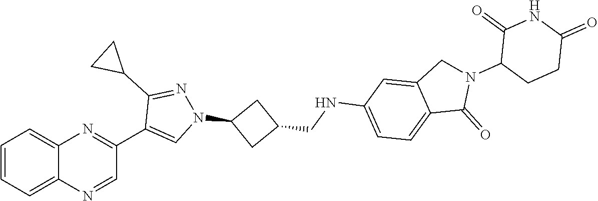 [SMILES: O=C1CCC(N2Cc3cc(NC[C@H]4C[C@H](n5cc(-c6cnc7ccccc7n6)c(C6CC6)n5)C4)ccc3C2=O)C(=O)N1] | 3-(5-(((trans-3-(3-cyclopropyl-4- (quinoxalin-2-yl)-1H-pyrazol-1- yl)cyclobutyl)methyl)amino)-1- oxoisoindolin-2-yl)piperidine-2,6-dione |
| GS- 652 | 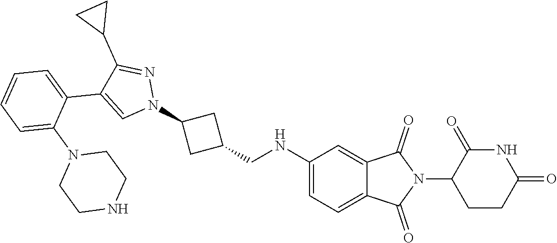 [SMILES: O=C1CCC(N2C(=O)c3ccc(NC[C@H]4C[C@H](n5cc(-c6ccccc6N6CCNCC6)c(C6CC6)n5)C4)cc3C2=O)C(=O)N1] | 5-(((trans-3-(3-cyclopropyl-4-(2- (piperazin-1-yl)phenyl)-1 H-pyrazol-1- yl)cyclobutyl)methyl)amino)-2-(2,6- dioxopiperidin-3-yl)isoindoline-1,3- dione |
| GS- 653 | 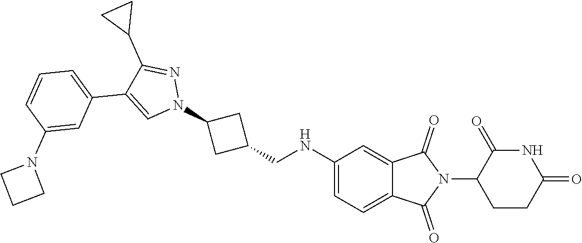 [SMILES: O=C1CCC(N2C(=O)c3ccc(NC[C@H]4C[C@H](n5cc(-c6cccc(N7CCC7)c6)c(C6CC6)n5)C4)cc3C2=O)C(=O)N1] | 5-(((trans-3-(4-(3-(azetidin-1-yl)phenyl)- 3-cyclopropyl-1 H-pyrazol-1- yl)cyclobutyl)methyl)amino)-2-(2,6- dioxopiperidin-3-yl)isoindoline-1,3- dione |
| GS- 654 | 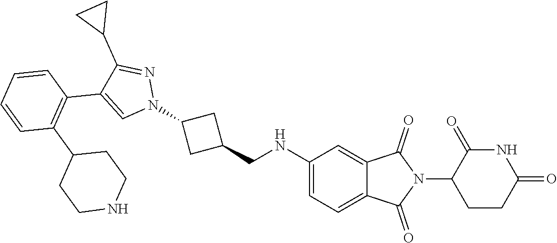 [SMILES: O=C1CCC(N2C(=O)c3ccc(NC[C@H]4C[C@H](n5cc(-c6ccccc6C6CCNCC6)c(C6CC6)n5)C4)cc3C2=O)C(=O)N1] | 5-(((trans-3-(3-cyclopropyl-4-(2- (piperidin-4-yl)phenyl)-1H-pyrazol-1- yl)cyclobutyl)methyl)amino)-2-(2,6- dioxopiperidin-3-yl)isoindoline-1,3- dione |
| GS- 655 | 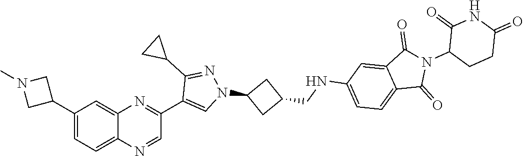 [SMILES: CN1CC(c2ccc3ncc(-c4cn([C@H]5C[C@H](CNc6ccc7c(c6)C(=O)N(C6CCC(=O)NC6=O)C7=O)C5)nc4C4CC4)nc3c2)C1] | 5-(((trans-3-(3-cyclopropyl-4-(7-(1- methylazetidin-3-yl)quinoxalin-2-yl)- 1H-pyrazol-1- yl)cyclobutyl)methyl)amino)-2-(2,6- dioxopiperidin-3-yl)isoindoline-1,3- dione |
| GS- 656 | 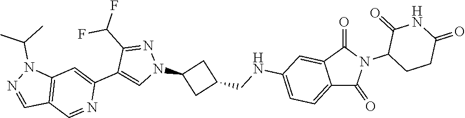 [SMILES: CC(C)n1ncc2cnc(-c3cn([C@H]4C[C@H](CNc5ccc6c(c5)C(=O)N(C5CCC(=O)NC5=O)C6=O)C4)nc3C(F)F)cc21] | 5-(((trans-3-(3-(difluoromethyl)-4-(1- isopropyl-1H-pyrazolo[4,3-c]pyridin-6- yl)-1H-pyrazol-1- yl)cyclobutyl)methyl)amino)-2-(2,6- dioxopiperidin-3-yl)isoindoline-1,3- dione |
| GS- 657 | 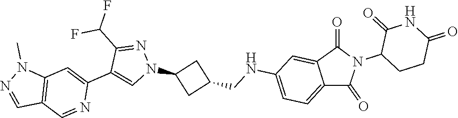 [SMILES: Cn1ncc2cnc(-c3cn([C@H]4C[C@H](CNc5ccc6c(c5)C(=O)N(C5CCC(=O)NC5=O)C6=O)C4)nc3C(F)F)cc21] | 5-(((trans-3-(3-(difluoromethyl)-4-(1- methyl-1H-pyrazolo[4,3-c]pyridin-6-yl)- 1H-pyrazol-1- yl)cyclobutyl)methyl)amino)-2-(2,6- dioxopiperidin-3-yl)isoindoline-1,3- dione |
| GS- 658 | 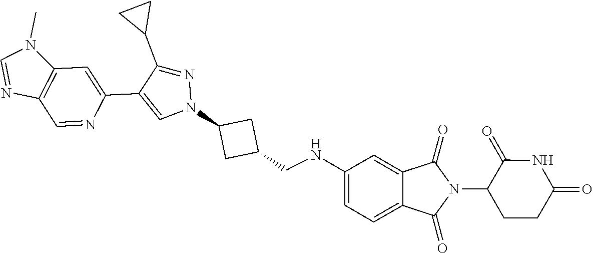 [SMILES: Cn1cnc2cnc(-c3cn([C@H]4C[C@H](CNc5ccc6c(c5)C(=O)N(C5CCC(=O)NC5=O)C6=O)C4)nc3C3CC3)cc21] | 5-(((trans-3-(3-cyclopropyl-4-(1-methyl- 1H-imidazo[4,5-c]pyridin-6-yl)-1H- pyrazol-1-yl)cyclobutyl)methyl)amino)- 2-(2,6-dioxopiperidin-3-yl)isoindoline- 1,3-dione |
| GS- 659 | 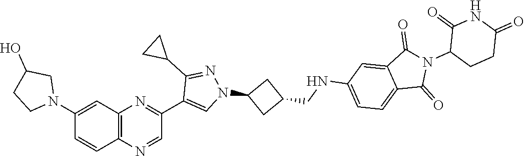 [SMILES: O=C1CCC(N2C(=O)c3ccc(NC[C@H]4C[C@H](n5cc(-c6cnc7ccc(N8CCC(O)C8)cc7n6)c(C6CC6)n5)C4)cc3C2=O)C(=O)N1] | 5-(((trans-3-(3-cyclopropyl-4-(7-(3- hydroxypyrrolidin-1-yl)quinoxalin-2-yl)- 1 H-pyrazol-1- yl)cyclobutyl)methyl)amino)-2-(2,6- dioxopiperidin-3-yl)isoindoline-1,3- dione |
| GS- 660 | 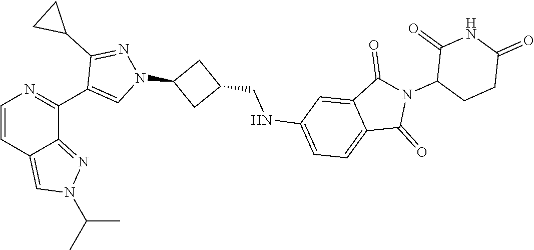 [SMILES: CC(C)n1cc2ccnc(-c3cn([C@H]4C[C@H](CNc5ccc6c(c5)C(=O)N(C5CCC(=O)NC5=O)C6=O)C4)nc3C3CC3)c2n1] | 5-(((trans-3-(3-cyclopropyl-4-(2- isopropyl-2H-pyrazolo[3,4-c]pyridin-7- yl)-1 H-pyrazol-1- yl)cyclobutyl)methyl)amino)-2-(2,6- dioxopiperidin-3-yl)isoindoline-1,3- dione |
| GS- 661 | 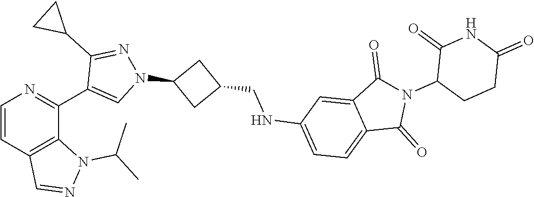 [SMILES: CC(C)n1ncc2ccnc(-c3cn([C@H]4C[C@H](CNc5ccc6c(c5)C(=O)N(C5CCC(=O)NC5=O)C6=O)C4)nc3C3CC3)c21] | 5-(((trans-3-(3-cyclopropyl-4-(1- isopropyl-1H-pyrazolo[3,4-c]pyridin-7- yl)-1H-pyrazol-1- yl)cyclobutyl)methyl)amino)-2-(2,6- dioxopiperidin-3-yl)isoindoline-1,3- dione |
| GS- 662 | 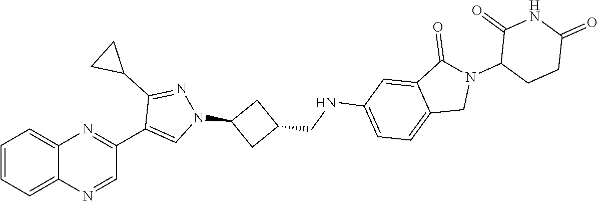 [SMILES: O=C1CCC(N2Cc3ccc(NC[C@H]4C[C@H](n5cc(-c6cnc7ccccc7n6)c(C6CC6)n5)C4)cc3C2=O)C(=O)N1] | 3-(6-(((trans-3-(3-cyclopropyl-4- (quinoxalin-2-yl)-1H-pyrazol-1- yl)cyclobutyl)methyl)amino)-1- oxoisoindolin-2-yl)piperidine-2,6-dione |
| GS- 663 | 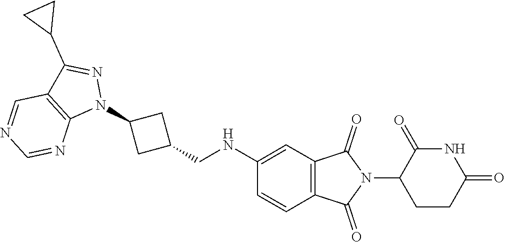 [SMILES: O=C1CCC(N2C(=O)c3ccc(NC[C@H]4C[C@H](n5nc(C6CC6)c6cncnc65)C4)cc3C2=O)C(=O)N1] | 5-(((trans-3-(3-cyclopropyl-1H- pyrazolo[3,4-d]pyrimidin-1- yl)cyclobutyl)methyl)amino)-2-(2,6- dioxopiperidin-3-yl)isoindoline-1,3- dione |
| GS- 664 | 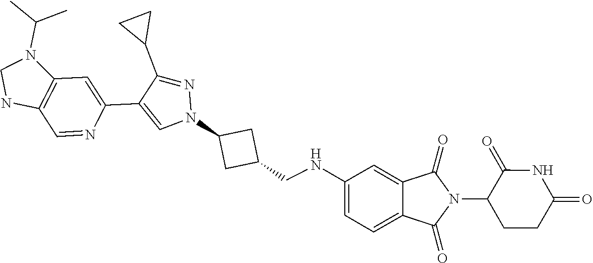 | 5-(((trans-3-(3-cyclopropyl-4-(1- isopropyl-1H-imidazo[4,5-c]pyridin-6- yl)-1H-pyrazol-1- yl)cyclobutyl)methyl)amino)-2-(2,6- dioxopiperidin-3-yl)isoindoline-1,3- dione |
| GS- 665 | 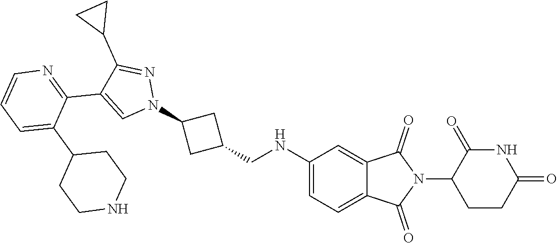 [SMILES: O=C1CCC(N2C(=O)c3ccc(NC[C@H]4C[C@H](n5cc(-c6ncccc6C6CCNCC6)c(C6CC6)n5)C4)cc3C2=O)C(=O)N1] | 5-(((trans-3-(3-cyclopropyl-4-(3- (piperidin-4-yl)pyridin-2-yl)-1H-pyrazol- 1-yl)cyclobutyl)methyl)amino)-2-(2,6- dioxopiperidin-3-yl)isoindoline-1,3- dione |
| GS- 666 | 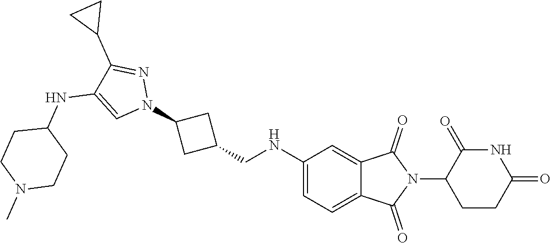 [SMILES: CN1CCC(Nc2cn([C@H]3C[C@H](CNc4ccc5c(c4)C(=O)N(C4CCC(=O)NC4=O)C5=O)C3)nc2C2CC2)CC1] | 5-(((trans-3-(3-cyclopropyl-4-((1- methylpiperidin-4-yl)amino)-1H- pyrazol-1-yl)cyclobutyl)methyl)amino)- 2-(2,6-dioxopiperidin-3-yl)isoindoline- 1,3-dione |
| GS- 667 | 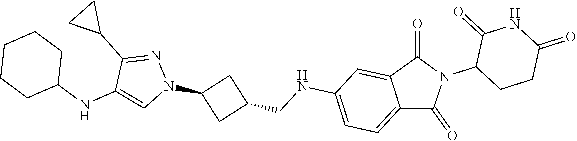 [SMILES: O=C1CCC(N2C(=O)c3ccc(NC[C@H]4C[C@H](n5cc(NC6CCCCC6)c(C6CC6)n5)C4)cc3C2=O)C(=O)N1] | 5-(((trans-3-(4-(cyclohexylamino)-3- cyclopropyl-1 H-pyrazol-1- yl)cyclobutyl)methyl)amino)-2-(2,6- dioxopiperidin-3-yl)isoindoline-1,3- dione |
| GS- 668 | 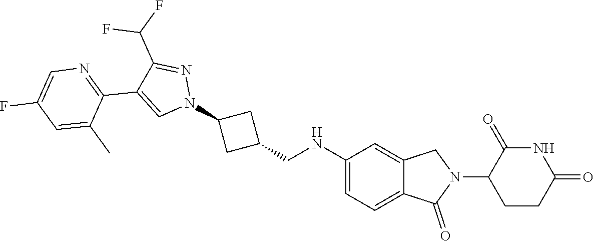 [SMILES: Cc1cc(F)cnc1-c1cn([C@H]2C[C@H](CNc3ccc4c(c3)CN(C3CCC(=O)NC3=O)C4=O)C2)nc1C(F)F] | 3-(5-(((trans-3-(3-(difluoromethyl)-4-(5- fluoro-3-methylpyridin-2-yl)-1H- pyrazol-1-yl)cyclobutyl)methyl)amino)- 1-oxoisoindolin-2-yl)piperidine-2,6- dione |
| GS- 669 | 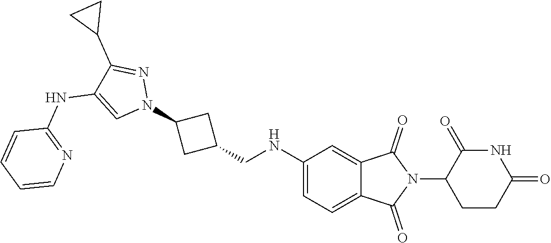 [SMILES: O=C1CCC(N2C(=O)c3ccc(NC[C@H]4C[C@H](n5cc(Nc6ccccn6)c(C6CC6)n5)C4)cc3C2=O)C(=O)N1] | 5-(((trans-3-(3-cyclopropyl-4-(pyridin-2- ylamino)-1H-pyrazol-1- yl)cyclobutyl)methyl)amino)-2-(2,6- dioxopiperidin-3-yl)isoindoline-1,3- dione |
| GS- 670 | 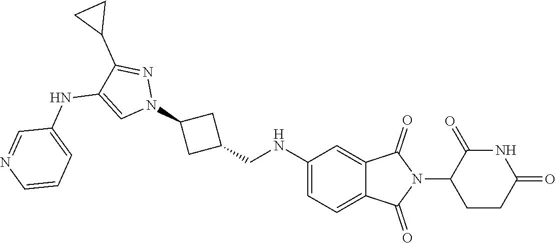 [SMILES: O=C1CCC(N2C(=O)c3ccc(NC[C@H]4C[C@H](n5cc(Nc6cccnc6)c(C6CC6)n5)C4)cc3C2=O)C(=O)N1] | 5-(((trans-3-(3-cyclopropyl-4-(pyridin-3- ylamino)-1H-pyrazol-1- yl)cyclobutyl)methyl)amino)-2-(2,6- dioxopiperidin-3-yl)isoindoline-1,3- dione |
| GS- 671 | 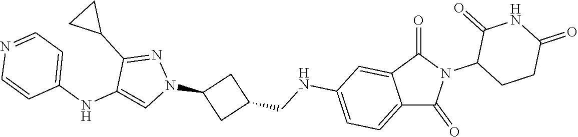 [SMILES: O=C1CCC(N2C(=O)c3ccc(NC[C@H]4C[C@H](n5cc(Nc6ccncc6)c(C6CC6)n5)C4)cc3C2=O)C(=O)N1] | 5-(((trans-3-(3-cyclopropyl-4-(pyridin-4- ylamino)-1H-pyrazol-1- yl)cyclobutyl)methyl)amino)-2-(2,6- dioxopiperidin-3-yl)isoindoline-1,3- dione |
| GS- 672 | 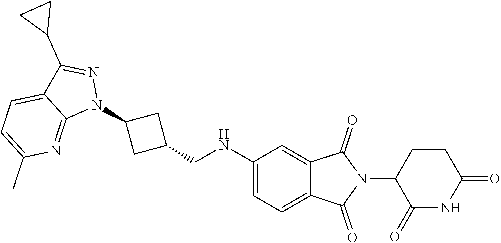 [SMILES: Cc1ccc2c(C3CC3)nn([C@H]3C[C@H](CNc4ccc5c(c4)C(=O)N(C4CCC(=O)NC4=O)C5=O)C3)c2n1] | 5-(((trans-3-(3-cyclopropyl-6-methyl- 1H-pyrazolo[3,4-b]pyridin-1- yl)cyclobutyl)methyl)amino)-2-(2,6- dioxopiperidin-3-yl)isoindoline-1,3- dione |
| GS- 673 | 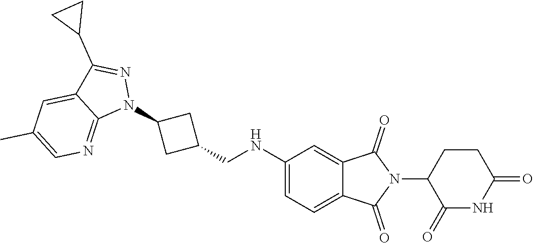 [SMILES: Cc1cnc2c(c1)c(C1CC1)nn2[C@H]1C[C@H](CNc2ccc3c(c2)C(=O)N(C2CCC(=O)NC2=O)C3=O)C1] | 5-(((trans-3-(3-cyclopropyl-5-methyl- 1H-pyrazolo[3,4-b]pyridin-1- yl)cyclobutyl)methyl)amino)-2-(2,6- dioxopiperidin-3-yl)isoindoline-1,3- dione |
| GS- 674 | 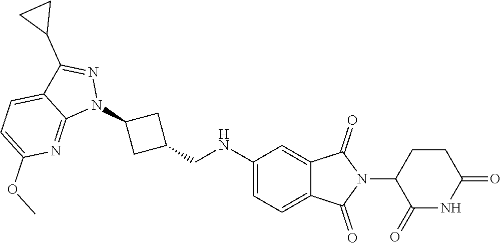 [SMILES: COc1ccc2c(C3CC3)nn([C@H]3C[C@H](CNc4ccc5c(c4)C(=O)N(C4CCC(=O)NC4=O)C5=O)C3)c2n1] | 5-(((trans-3-(3-cyclopropyl-6-methoxy- 1H-pyrazolo[3,4-b]pyridin-1- yl)cyclobutyl)methyl)amino)-2-(2,6- dioxopiperidin-3-yl)isoindoline-1,3- dione |
| GS- 675 | 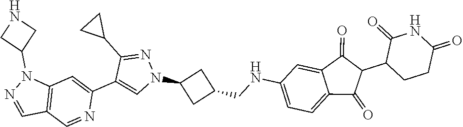 [SMILES: O=C1CCC(C2C(=O)c3ccc(NC[C@H]4C[C@H](n5cc(-c6cc7c(cn6)cnn7C6CNC6)c(C6CC6)n5)C4)cc3C2=O)C(=O)N1] | 5-(((trans-3-(4-(1-(azetidin-3-yl)-1H- pyrazolo[4,3-c]pyridin-6-yl)-3- cyclopropyl-1 H-pyrazol-1- yl)cyclobutyl)methyl)amino)-2-(2,6- dioxopiperidin-3-yl)isoindoline-1,3- dione |
| GS- 676 | 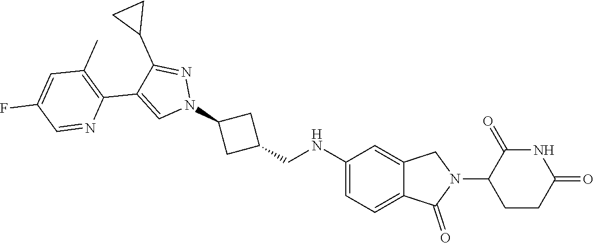 [SMILES: Cc1cc(F)cnc1-c1cn([C@H]2C[C@H](CNc3ccc4c(c3)CN(C3CCC(=O)NC3=O)C4=O)C2)nc1C1CC1] | 3-(5-(((trans-3-(3-cyclopropyl-4-(5- fluoro-3-methylpyridin-2-yl)-1H- pyrazol-1-yl)cyclobutyl)methyl)amino)- 1-oxoisoindolin-2-yl)piperidine-2,6- dione |
| GS- 677 | 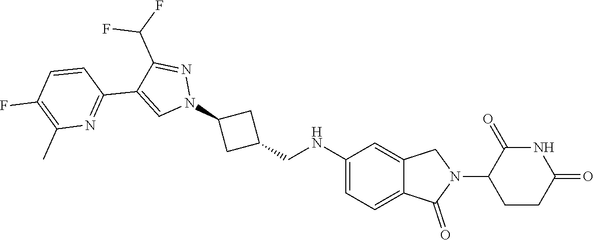 [SMILES: Cc1nc(-c2cn([C@H]3C[C@H](CNc4ccc5c(c4)CN(C4CCC(=O)NC4=O)C5=O)C3)nc2C(F)F)ccc1F] | 3-(5-(((trans-3-(3-(difluoromethyl)-4-(5- fluoro-6-methylpyridin-2-yl)-1H- pyrazol-1-yl)cyclobutyl)methyl)amino)- 1-oxoisoindolin-2-yl)piperidine-2,6- dione |
| GS- 678 | 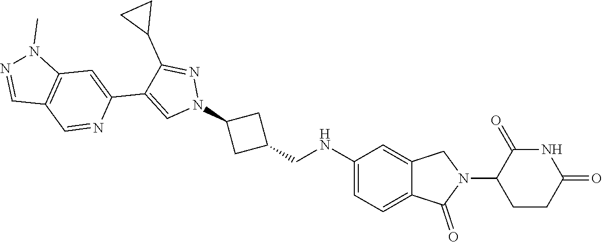 [SMILES: Cn1ncc2cnc(-c3cn([C@H]4C[C@H](CNc5ccc6c(c5)CN(C5CCC(=O)NC5=O)C6=O)C4)nc3C3CC3)cc21] | 3-(5-(((trans-3-(3-cyclopropyl-4-(1- methyl-1H-pyrazolo[4,3-c]pyridin-6-yl)- 1H-pyrazol-1- yl)cyclobutyl)methyl)amino)-1- oxoisoindolin-2-yl)piperidine-2,6-dione |
| GS- 679 | 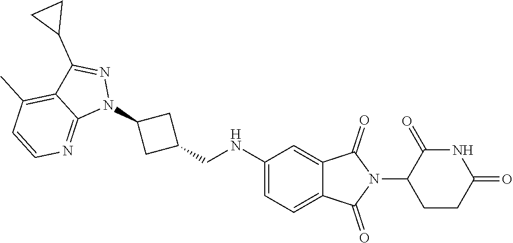 [SMILES: Cc1ccnc2c1c(C1CC1)nn2[C@H]1C[C@H](CNc2ccc3c(c2)C(=O)N(C2CCC(=O)NC2=O)C3=O)C1] | 5-(((trans-3-(3-cyclopropyl-4-methyl- 1H-pyrazolo[3,4-b]pyridin-1- yl)cyclobutyl)methyl)amino)-2-(2,6- dioxopiperidin-3-yl)isoindoline-1,3- dione |
| GS- 680 | 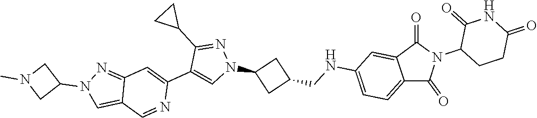 [SMILES: CN1CC(n2cc3cnc(-c4cn([C@H]5C[C@H](CNc6ccc7c(c6)C(=O)N(C6CCC(=O)NC6=O)C7=O)C5)nc4C4CC4)cc3n2)C1] | 5-(((trans-3-(3-cyclopropyl-4-(2-(1- methylazetidin-3-yl)-2H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 681 | 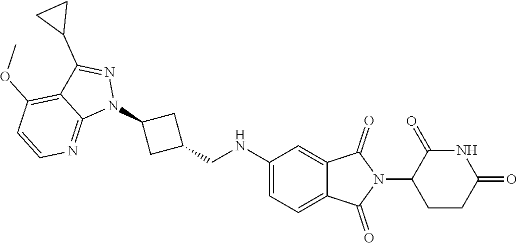 [SMILES: COc1ccnc2c1c(C1CC1)nn2[C@H]1C[C@H](CNc2ccc3c(c2)C(=O)N(C2CCC(=O)NC2=O)C3=O)C1] | 5-(((trans-3-(3-cyclopropyl-4-methoxy- 1H-pyrazolo[3,4-b]pyridin-1- yl)cyclobutyl)methyl)amino)-2-(2,6- dioxopiperidin-3-yl)isoindoline-1,3- dione |
| GS- 682 | 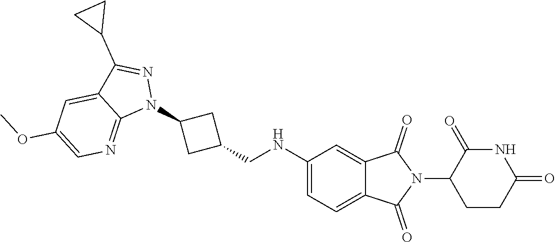 [SMILES: COc1cnc2c(c1)c(C1CC1)nn2[C@H]1C[C@H](CNc2ccc3c(c2)C(=O)N(C2CCC(=O)NC2=O)C3=O)C1] | 5-(((trans-3-(3-cyclopropyl-5-methoxy- 1H-pyrazolo[3,4-b]pyridin-1- yl)cyclobutyl)methyl)amino)-2-(2,6- dioxopiperidin-3-yl)isoindoline-1,3- dione |
| GS- 683 | 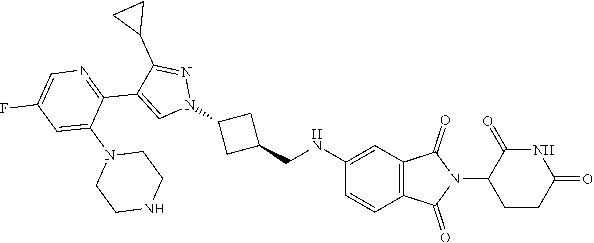 [SMILES: O=C1CCC(N2C(=O)c3ccc(NC[C@H]4C[C@H](n5cc(-c6ncc(F)cc6N6CCNCC6)c(C6CC6)n5)C4)cc3C2=O)C(=O)N1] | 5-(((trans-3-(3-cyclopropyl-4-(5-fluoro- 3-(piperazin-1-yl)pyridin-2-yl)-1H- pyrazol-1-yl)cyclobutyl)methyl)amino)- 2-(2,6-dioxopiperidin-3-yl)isoindoline- 1,3-dione |
| GS- 684 | 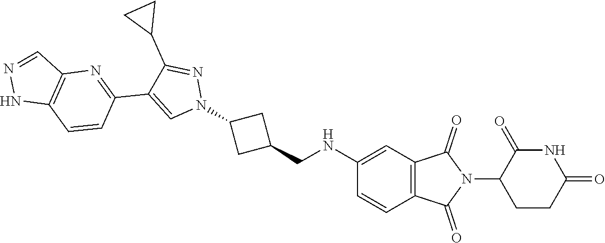 [SMILES: O=C1CCC(N2C(=O)c3ccc(NC[C@H]4C[C@H](n5cc(-c6ccc7[nH]ncc7n6)c(C6CC6)n5)C4)cc3C2=O)C(=O)N1] | 5-(((trans-3-(3-cyclopropyl-4-(1H- pyrazolo[4,3-b]pyridin-5-yl)-1H-pyrazol- 1-yl)cyclobutyl)methyl)amino)-2-(2,6- dioxopiperidin-3-yl)isoindoline-1,3- dione |
| GS- 685 | 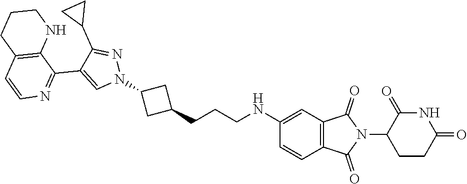 [SMILES: O=C1CCC(N2C(=O)c3ccc(NCCC[C@H]4C[C@H](n5cc(-c6nccc7c6NCCC7)c(C6CC6)n5)C4)cc3C2=O)C(=O)N1] | 5-((3-((1r,3s)-3-(3-cyclopropyl-4- (1,2,3,4-tetrahydro-1,7-naphthyridin-8- yl)-1 H-pyrazol-1- yl)cyclobutyl)propyl)amino)-2-(2,6- dioxopiperidin-3-yl)isoindoline-1,3- dione |
| GS- 686 | 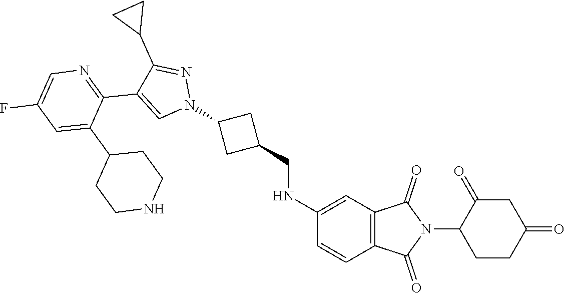 [SMILES: O=C1CCC(N2C(=O)c3ccc(NC[C@H]4C[C@H](n5cc(-c6ncc(F)cc6C6CCNCC6)c(C6CC6)n5)C4)cc3C2=O)C(=O)C1] | 5-(((trans-3-(3-cyclopropyl-4-(5-fluoro- 3-(piperidin-4-yl)pyridin-2-yl)-1H- pyrazol-1-yl)cyclobutyl)methyl)amino)- 2-(2,4-dioxocyclohexyl)isoindoline-1,3- dione |
| GS- 687 | 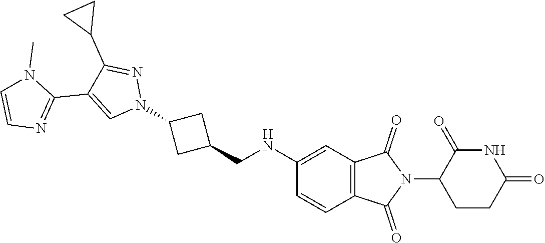 [SMILES: Cn1ccnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(1-methyl- 1H-imidazol-2-yl)-1H-pyrazol-1- yl)cyclobutyl)methyl)amino)-2-(2,6- dioxopiperidin-3-yl)isoindoline-1,3- dione |
| GS- 688 | 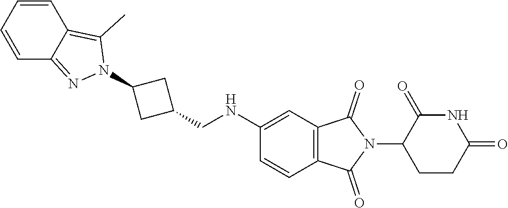 [SMILES: Cc1c2ccccc2nn1[C@H]1C[C@H](CNc2ccc3c(c2)C(=O)N(C2CCC(=O)NC2=O)C3=O)C1] | 2-(2,6-dioxopiperidin-3-yl)-5-(((trans-3- (3-methyl-2/-indazol-2- yl)cyclobutyl)methyl)amino)isoindoline- 1,3-dione |
| GS- 689 | 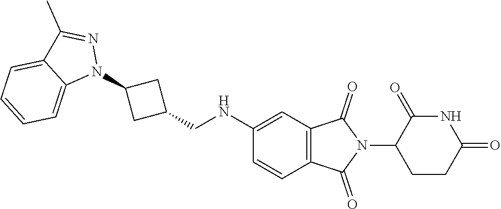 [SMILES: Cc1nn([C@H]2C[C@H](CNc3ccc4c(c3)C(=O)N(C3CCC(=O)NC3=O)C4=O)C2)c2ccccc12] | 2-(2,6-dioxopiperidin-3-yl)-5-(((trans-3- (3-methyl-1H-indazol-1- yl)cyclobutyl)methyl)amino)isoindoline- 1,3-dione |
| GS- 690 | 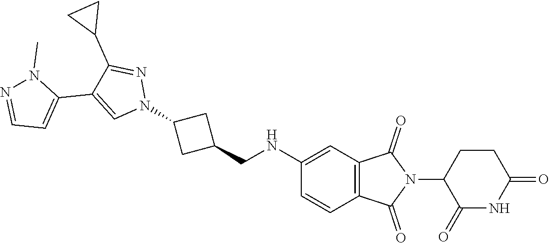 [SMILES: Cn1nccc1-c1cn([C@H]2C[C@H](CNc3ccc4c(c3)C(=O)N(C3CCC(=O)NC3=O)C4=O)C2)nc1C1CC1] | 5-(((trans-3-(3'-cyclopropyl-2-methyl- 1'H,2H-[3,4'-bipyrazol]-1'- yl)cyclobutyl)methyl)amino)-2-(2,6- dioxopiperidin-3-yl)isoindoline-1,3- dione |
| GS- 691 | 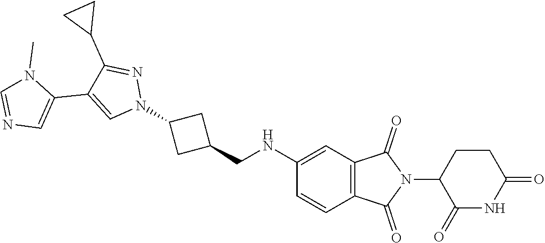 [SMILES: Cn1cncc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(1-methyl- 1H-imidazol-5-yl)-1 H-pyrazol-1- yl)cyclobutyl)methyl)amino)-2-(2,6- dioxopiperidin-3-yl)isoindoline-1,3- dione |
| GS- 693 | 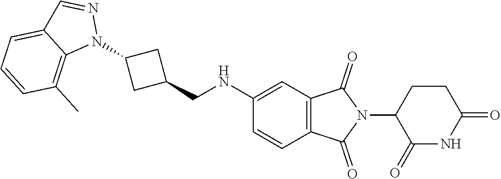 [SMILES: Cc1cccc2cnn([C@H]3C[C@H](CNc4ccc5c(c4)C(=O)N(C4CCC(=O)NC4=O)C5=O)C3)c12] | 2-(2,6-dioxopiperidin-3-yl)-5-(((trans-3- (7-methyl-1H-indazol-1- yl)cyclobutyl)methyl)amino)isoindoline- 1,3-dione |
| GS- 694 | 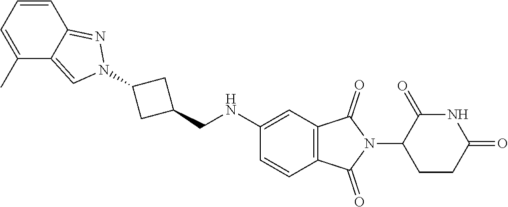 [SMILES: Cc1cccc2nn([C@H]3C[C@H](CNc4ccc5c(c4)C(=O)N(C4CCC(=O)NC4=O)C5=O)C3)cc12] | 2-(2,6-dioxopiperidin-3-yl)-5-(((trans-3- (7-methyl-2H-indazol-2- yl)cyclobutyl)methyl)amino)isoindoline- 1,3-dione |
| GS- 695 | 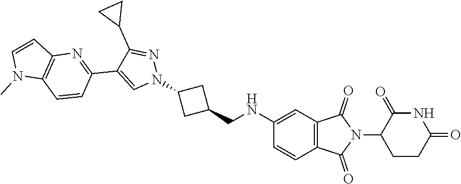 [SMILES: Cn1ccc2nc(-c3cn([C@H]4C[C@H](CNc5ccc6c(c5)C(=O)N(C5CCC(=O)NC5=O)C6=O)C4)nc3C3CC3)ccc21] | 5-(((trans-3-(3-cyclopropyl-4-(1-methyl- 1H-pyrrolo[3,2-b]pyridin-5-yl)-1H- pyrazol-1-yl)cyclobutyl)methyl)amino)- 2-(2,6-dioxopiperidin-3-yl)isoindoline- 1,3-dione |
| GS- 696 | 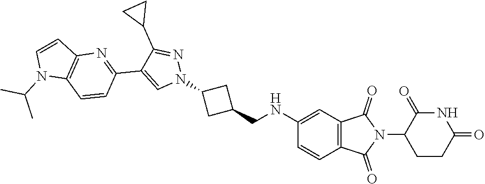 [SMILES: CC(C)n1ccc2nc(-c3cn([C@H]4C[C@H](CNc5ccc6c(c5)C(=O)N(C5CCC(=O)NC5=O)C6=O)C4)nc3C3CC3)ccc21] | 5-(((trans-3-(3-cyclopropyl-4-(1- isopropyl-1H-pyrrolo[3,2-b]pyridin-5- yl)-1H-pyrazol-1- yl)cyclobutyl)methyl)amino)-2-(2,6- dioxopiperidin-3-yl)isoindoline-1,3- dione |
| GS- 697 | 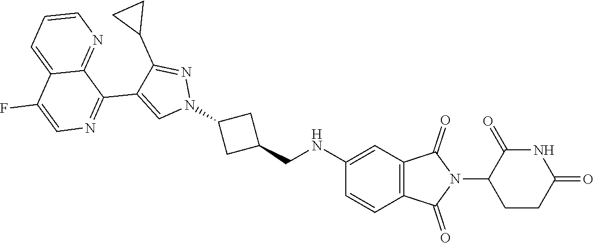 [SMILES: O=C1CCC(N2C(=O)c3ccc(NC[C@H]4C[C@H](n5cc(-c6ncc(F)c7cccnc67)c(C6CC6)n5)C4)cc3C2=O)C(=O)N1] | 5-(((trans-3-(3-cyclopropyl-4-(5-fluoro- 1,7-naphthyridin-8-yl)-1H-pyrazol-1- yl)cyclobutyl)methyl)amino)-2-(2,6- dioxopiperidin-3-yl)isoindoline-1,3- dione |
| GS- 699 | 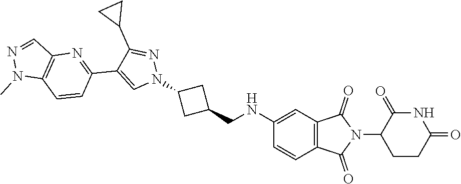 [SMILES: Cn1ncc2nc(-c3cn([C@H]4C[C@H](CNc5ccc6c(c5)C(=O)N(C5CCC(=O)NC5=O)C6=O)C4)nc3C3CC3)ccc21] | 5-(((trans-3-(3-cyclopropyl-4-(1-methyl- 1H-pyrazolo[4,3-b]pyridin-5-yl)-1H- pyrazol-1-yl)cyclobutyl)methyl)amino)- 2-(2,6-dioxopiperidin-3-yl)isoindoline- 1,3-dione |
| GS- 700 | 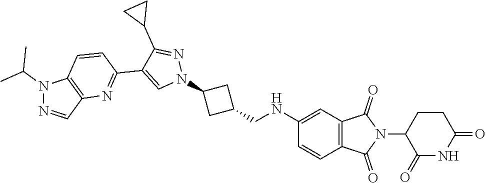 [SMILES: CC(C)n1ncc2nc(-c3cn([C@H]4C[C@H](CNc5ccc6c(c5)C(=O)N(C5CCC(=O)NC5=O)C6=O)C4)nc3C3CC3)ccc21] | 5-(((trans-3-(3-cyclopropyl-4-(1- isopropyl-1H-pyrazolo[4,3-b]pyridin-5- yl)-1H-pyrazol-1- yl)cyclobutyl)methyl)amino)-2-(2,6- dioxopiperidin-3-yl)isoindoline-1,3- dione |
| GS- 701 | 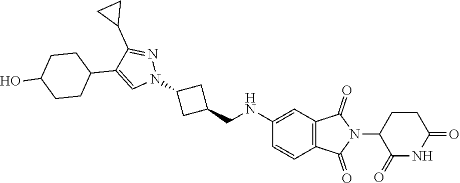 [SMILES: O=C1CCC(N2C(=O)c3ccc(NC[C@H]4C[C@H](n5cc(C6CCC(O)CC6)c(C6CC6)n5)C4)cc3C2=O)C(=O)N1] | 5-(((trans-3-(3-cyclopropyl-4-(4- hydroxycyclohexyl)-1H-pyrazol-1- yl)cyclobutyl)methyl)amino)-2-(2,6- dioxopiperidin-3-yl)isoindoline-1,3- dione |
| GS- 702 | 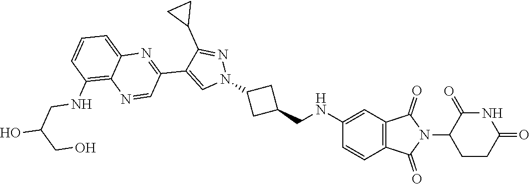 | 5-(((trans-3-(3-cyclopropyl-4-(5-((2,3- dihydroxypropyl)amino)quinoxalin-2- yl)-1H-pyrazol-1- yl)cyclobutyl)methyl)amino)-2-(2,6- dioxopiperidin-3-yl)isoindoline-1,3- dione |
| GS- 703 | 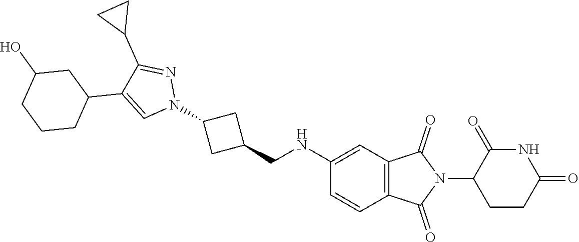 [SMILES: O=C1CCC(N2C(=O)c3ccc(NC[C@H]4C[C@H](n5cc(C6CCCC(O)C6)c(C6CC6)n5)C4)cc3C2=O)C(=O)N1] | 5-(((trans-3-(3-cyclopropyl-4-(3- hydroxycyclohexyl)-1H-pyrazol-1- yl)cyclobutyl)methyl)amino)-2-(2,6- dioxopiperidin-3-yl)isoindoline-1,3- dione |
| GS- 704 | 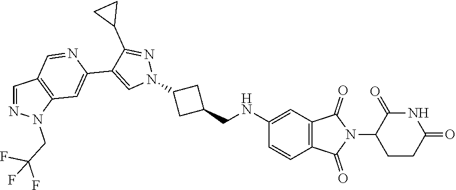 | 5-(((trans-3-(3-cyclopropyl-4-(1-(2,2,2- trifluoroethyl)-1H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 705 | 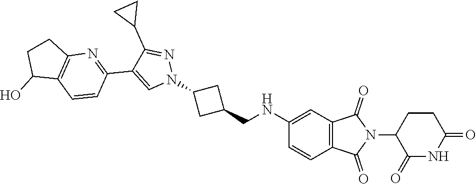 [SMILES: O=C1CCC(N2C(=O)c3ccc(NC[C@H]4C[C@H](n5cc(-c6ccc7c(n6)CCC7O)c(C6CC6)n5)C4)cc3C2=O)C(=O)N1] | 5-(((trans-3-(3-cyclopropyl-4-(5- hydroxy-6,7-dihydro-5H- cyclopenta[b]pyridin-2-yl)-1H-pyrazol- 1-yl)cyclobutyl)methyl)amino)-2-(2,6- dioxopiperidin-3-yl)isoindoline-1,3- dione |
| GS- 706 | 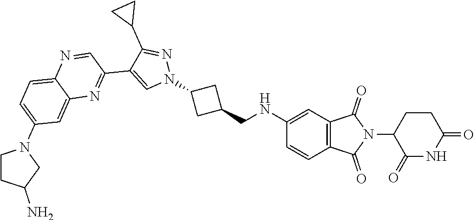 [SMILES: NC1CCN(c2ccc3ncc(-c4cn([C@H]5C[C@H](CNc6ccc7c(c6)C(=O)N(C6CCC(=O)NC6=O)C7=O)C5)nc4C4CC4)nc3c2)C1] | 5-(((trans-3-(4-(7-(3-aminopyrrolidin-1- yl)quinoxalin-2-yl)-3-cyclopropyl-1H- pyrazol-1-yl)cyclobutyl)methyl)amino)- 2-(2,6-dioxopiperidin-3-yl)isoindoline- 1,3-dione |
| GS- 707 | 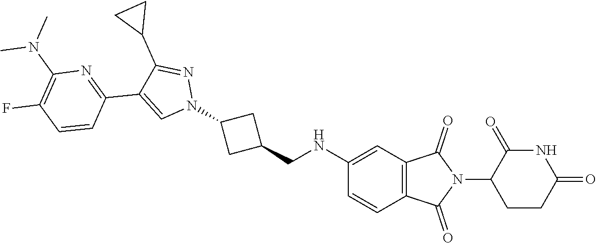 [SMILES: CN(C)c1nc(-c2cn([C@H]3C[C@H](CNc4ccc5c(c4)C(=O)N(C4CCC(=O)NC4=O)C5=O)C3)nc2C2CC2)ccc1F] | 5-(((trans-3-(3-cyclopropyl-4-(6- (dimethylamino)-5-fluoropyridin-2-yl)- 1H-pyrazol-1- yl)cyclobutyl)methyl)amino)-2-(2,6- dioxopiperidin-3-yl)isoindoline-1,3- dione |
| GS- 708 | 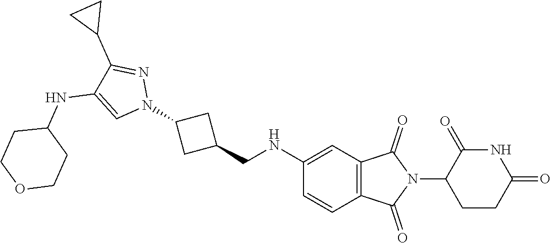 [SMILES: O=C1CCC(N2C(=O)c3ccc(NC[C@H]4C[C@H](n5cc(NC6CCOCC6)c(C6CC6)n5)C4)cc3C2=O)C(=O)N1] | 5-(((trans-3-(3-cyclopropyl-4- ((tetrahydro-2/I-pyran-4-yl)amino)-1H- pyrazol-1-yl)cyclobutyl)methyl)amino)- 2-(2,6-dioxopiperidin-3-yl)isoindoline- 1,3-dione |
| GS- 709 | 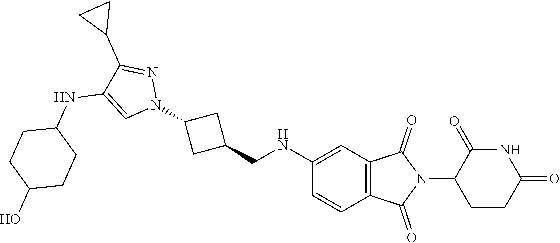 [SMILES: O=C1CCC(N2C(=O)c3ccc(NC[C@H]4C[C@H](n5cc(NC6CCC(O)CC6)c(C6CC6)n5)C4)cc3C2=O)C(=O)N1] | 5-(((trans-3-(3-cyclopropyl-4-((4- hydroxycyclohexyl)amino)-1H-pyrazol- 1-yl)cyclobutyl)methyl)amino)-2-(2,6- dioxopiperidin-3-yl)isoindoline-1,3- dione |
| GS- 710 | 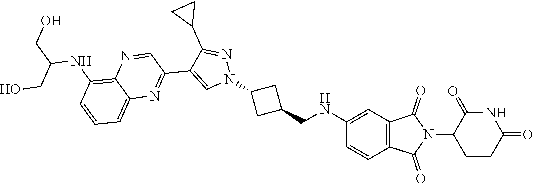 [SMILES: O=C1CCC(N2C(=O)c3ccc(NC[C@H]4C[C@H](n5cc(-c6cnc7c(NC(CO)CO)cccc7n6)c(C6CC6)n5)C4)cc3C2=O)C(=O)N1] | 5-(((trans-3-(3-cyclopropyl-4-(5-((1,3- dihydroxypropan-2-yl)amino)quinoxalin- 2-yl)-1H-pyrazol-1- yl)cyclobutyl)methyl)amino)-2-(2,6- dioxopiperidin-3-yl)isoindoline-1,3- dione |
| GS- 711 | 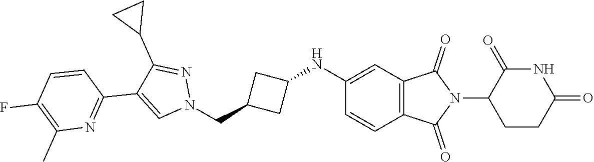 [SMILES: Cc1nc(-c2cn(C[C@H]3C[C@H](Nc4ccc5c(c4)C(=O)N(C4CCC(=O)NC4=O)C5=O)C3)nc2C2CC2)ccc1F] | 5-((trans-3-((3-cyclopropyl-4-(5-fluoro- 6-methylpyridin-2-yl)-1H-pyrazol-1- yl)methyl)cyclobutyl)amino)-2-(2,6- dioxopiperidin-3-yl)isoindoline-1,3- dione |
| GS- 712 | 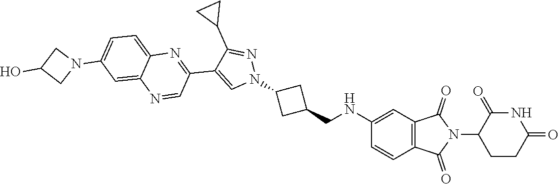 [SMILES: O=C1CCC(N2C(=O)c3ccc(NC[C@H]4C[C@H](n5cc(-c6cnc7cc(N8CC(O)C8)ccc7n6)c(C6CC6)n5)C4)cc3C2=O)C(=O)N1] | 5-(((trans-3-(3-cyclopropyl-4-(6-(3- hydroxyazetidin-1-yl)quinoxalin-2-yl)- 1H-pyrazol-1- yl)cyclobutyl)methyl)amino)-2-(2,6- dioxopiperidin-3-yl)isoindoline-1,3- dione |
| GS- 713 | 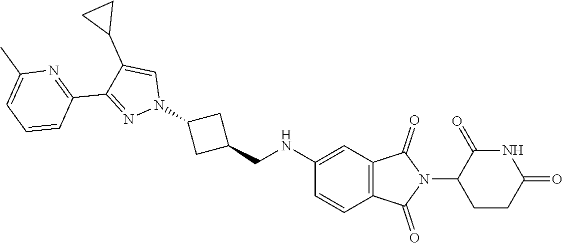 [SMILES: Cc1cccc(-c2nn([C@H]3C[C@H](CNc4ccc5c(c4)C(=O)N(C4CCC(=O)NC4=O)C5=O)C3)cc2C2CC2)n1] | 5-(((trans-3-(4-cyclopropyl-3-(6- methylpyridin-2-yl)-1 H-pyrazol-1- yl)cyclobutyl)methyl)amino)-2-(2,6- dioxopiperidin-3-yl)isoindoline-1,3- dione |
| GS- 714 | 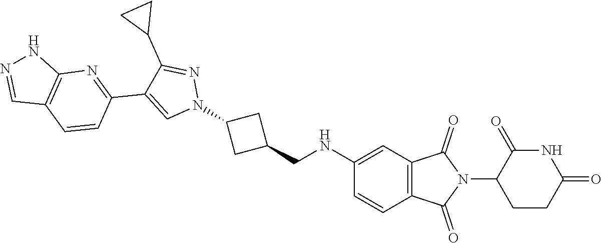 [SMILES: O=C1CCC(N2C(=O)c3ccc(NC[C@H]4C[C@H](n5cc(-c6ccc7cn[nH]c7n6)c(C6CC6)n5)C4)cc3C2=O)C(=O)N1] | 5-(((trans-3-(3-cyclopropyl-4-(1H- pyrazolo[3,4-b]pyridin-6-yl)-1H-pyrazol- 1-yl)cyclobutyl)methyl)amino)-2-(2,6- dioxopiperidin-3-yl)isoindoline-1,3- dione |
| GS- 715 | 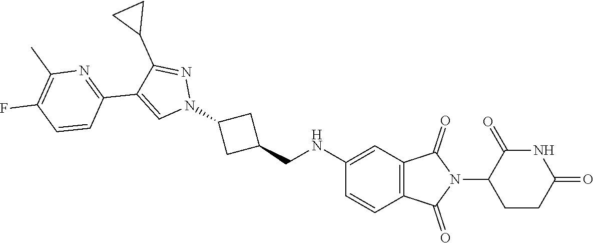 [SMILES: Cc1nc(-c2cn([C@H]3C[C@H](CNc4ccc5c(c4)C(=O)N(C4CCC(=O)NC4=O)C5=O)C3)nc2C2CC2)ccc1F] | 5-((trans-3-(3-cyclopropyl-4-(5-fluoro-6- methylpyridin-2-yl)-1H-pyrazol-1- yl)cyclobutyl)methoxy)-2-(2,6- dioxopiperidin-3-yl)isoindoline-1,3- dione |
| GS- 716 | 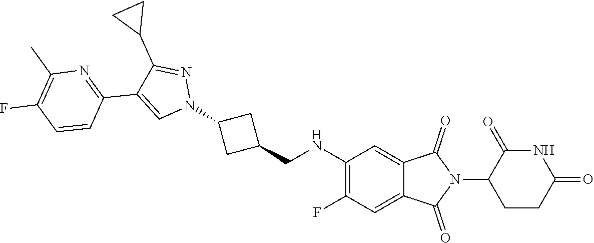 [SMILES: Cc1nc(-c2cn([C@H]3C[C@H](CNc4cc5c(cc4F)C(=O)N(C4CCC(=O)NC4=O)C5=O)C3)nc2C2CC2)ccc1F] | 5-(((trans-3-(3-cyclopropyl-4-(5-fluoro- 6-methylpyridin-2-yl)-1H-pyrazol-1- yl)cyclobutyl)methyl)amino)-2-(2,6- dioxopiperidin-3-yl)-6-fluoroisoindoline- 1,3-dione |
| GS- 717 | 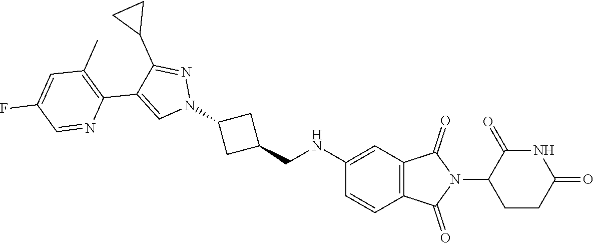 [SMILES: Cc1cc(F)cnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(5-fluoro- 3-methylpyridin-2-yl)-1/-pyrazol-1- yl)cyclobutyl)methyl)amino)-2-(2,6- dioxopiperidin-3-yl)isoindoline-1,3- dione |
| GS- 718 | 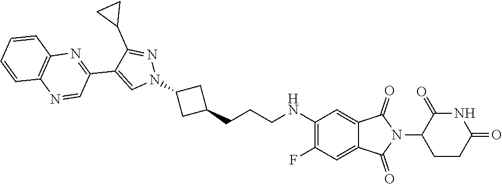 [SMILES: O=C1CCC(N2C(=O)c3cc(F)c(NCCC[C@H]4C[C@H](n5cc(-c6cnc7ccccc7n6)c(C6CC6)n5)C4)cc3C2=O)C(=O)N1] | 5-((3-(trans-3-(3-cyclopropyl-4- (quinoxalin-2-yl)-1/-pyrazol-1- yl)cyclobutyl)propyl)amino)-2-(2,6- dioxopiperidin-3-yl)-6-fluoroisoindoline- 1,3-dione |
| GS- 719 | 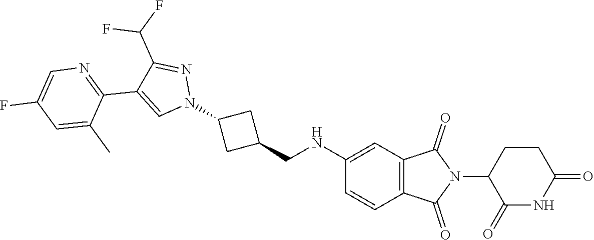 [SMILES: Cc1cc(F)cnc1-c1cn([C@H]2C[C@H](CNc3ccc4c(c3)C(=O)N(C3CCC(=O)NC3=O)C4=O)C2)nc1C(F)F] | 5-(((trans-3-(3-(difluoromethyl)-4-(5- fluoro-3-methylpyridin-2-yl)-1H- pyrazol-1-yl)cyclobutyl)methyl)amino)- 2-(2,6-dioxopiperidin-3-yl)isoindoline- 1,3-dione |
| GS- 720 | 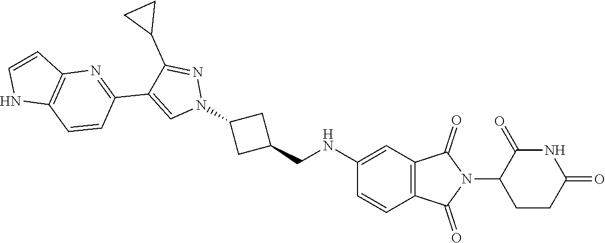 [SMILES: O=C1CCC(N2C(=O)c3ccc(NC[C@H]4C[C@H](n5cc(-c6ccc7[nH]ccc7n6)c(C6CC6)n5)C4)cc3C2=O)C(=O)N1] | 5-(((trans-3-(3-cyclopropyl-4-(1H- pyrrolo[3,2-b]pyridin-5-yl)-1H-pyrazol- 1-yl)cyclobutyl)methyl)amino)-2-(2,6- dioxopiperidin-3-yl)isoindoline-1,3- dione |
| GS- 721 | 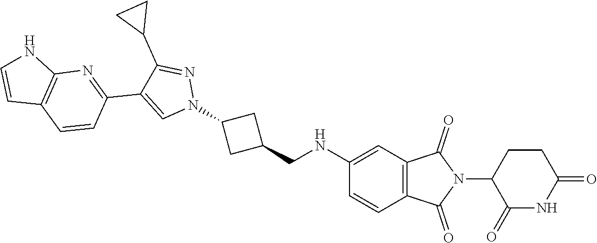 [SMILES: O=C1CCC(N2C(=O)c3ccc(NC[C@H]4C[C@H](n5cc(-c6ccc7cc[nH]c7n6)c(C6CC6)n5)C4)cc3C2=O)C(=O)N1] | 5-(((trans-3-(3-cyclopropyl-4-(1H- pyrrolo[2,3-b]pyridin-6-yl)-1H-pyrazol- 1-yl)cyclobutyl)methyl)amino)-2-(2,6- dioxopiperidin-3-yl)isoindoline-1,3- dione |
| GS- 722 | 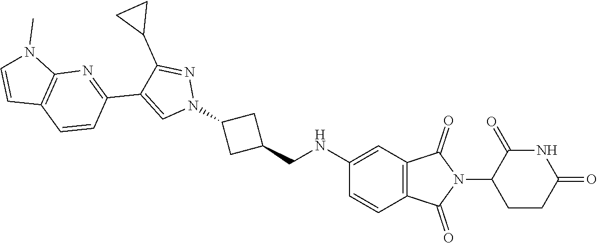 [SMILES: Cn1ccc2ccc(-c3cn([C@H]4C[C@H](CNc5ccc6c(c5)C(=O)N(C5CCC(=O)NC5=O)C6=O)C4)nc3C3CC3)nc21] | 5-(((trans-3-(3-cyclopropyl-4-(1-methyl- 1H-pyrrolo[2,3-b]pyridin-6-yl)-1H- pyrazol-1-yl)cyclobutyl)methyl)amino)- 2-(2,6-dioxopiperidin-3-yl)isoindoline- 1,3-dione |
| GS- 723 | 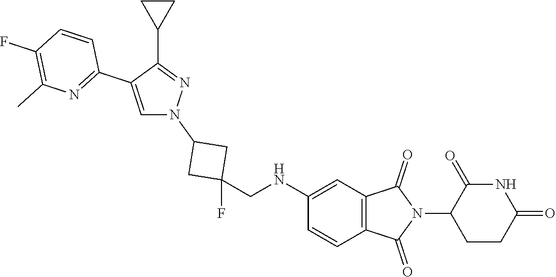 [SMILES: Cc1nc(-c2cn(C3CC(F)(CNc4ccc5c(c4)C(=O)N(C4CCC(=O)NC4=O)C5=O)C3)nc2C2CC2)ccc1F] | 5-(((3-(3-cyclopropyl-4-(5-fluoro-6- methylpyridin-2-yl)-1H-pyrazol-1-yl)-1- fluorocyclobutyl)methyl)amino)-2-(2,6- dioxopiperidin-3-yl)isoindoline-1,3- dione |
| GS- 724 | 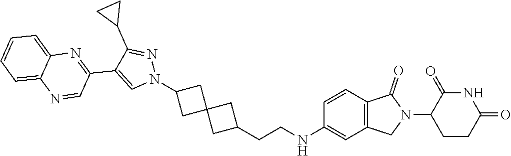 [SMILES: O=C1CCC(N2Cc3cc(NCCC4CC5(C4)CC(n4cc(-c6cnc7ccccc7n6)c(C6CC6)n4)C5)ccc3C2=O)C(=O)N1] | 5-((2-(6-(3-cyclopropyl-4-(quinoxalin-2- yl)-1H-pyrazol-1-yl)spiro[3.3]heptan-2- yl)ethyl)amino)-2-(2,6-dioxopiperidin-3- yl)isoindoline-1,3-dione |
| GS- 725 | 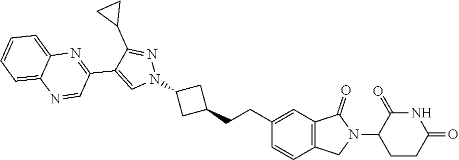 [SMILES: O=C1CCC(N2Cc3ccc(CC[C@H]4C[C@H](n5cc(-c6cnc7ccccc7n6)c(C6CC6)n5)C4)cc3C2=O)C(=O)N1] | 3-(6-(2-(trans-3-(3-cyclopropyl-4- (quinoxalin-2-yl)-1H-pyrazol-1- yl)cyclobutyl)ethyl)-1-oxoisoindolin-2- yl)piperidine-2,6-dione |
| GS- 726 | 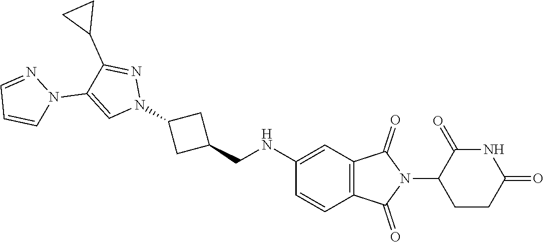 [SMILES: O=C1CCC(N2C(=O)c3ccc(NC[C@H]4C[C@H](n5cc(-n6cccn6)c(C6CC6)n5)C4)cc3C2=O)C(=O)N1] | 5-(((trans-3-(3'-cyclopropyl-1'H-[1,4'- bipyrazol]-1'- yl)cyclobutyl)methyl)amino)-2-(2,6- dioxopiperidin-3-yl)isoindoline-1,3- dione |
| GS- 727 | 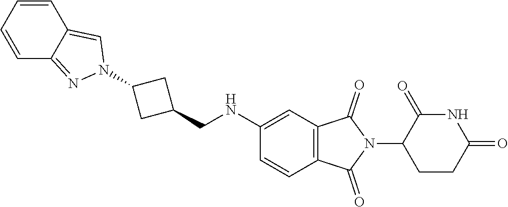 [SMILES: O=C1CCC(N2C(=O)c3ccc(NC[C@H]4C[C@H](n5cc6ccccc6n5)C4)cc3C2=O)C(=O)N1] | 5-(((trans-3-(2H-indazol-2- yl)cyclobutyl)methyl)amino)-2-(2,6- dioxopiperidin-3-yl)isoindoline-1,3- dione |
| GS- 728 | 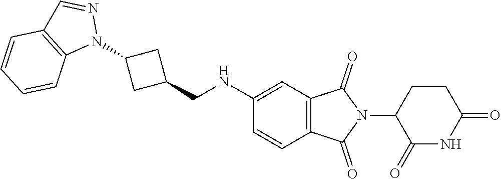 [SMILES: O=C1CCC(N2C(=O)c3ccc(NC[C@H]4C[C@H](n5ncc6ccccc65)C4)cc3C2=O)C(=O)N1] | 5-(((trans-3-(1H-indazol-1- yl)cyclobutyl)methyl)amino)-2-(2,6- dioxopiperidin-3-yl)isoindoline-1,3- dione |
| GS- 729 | 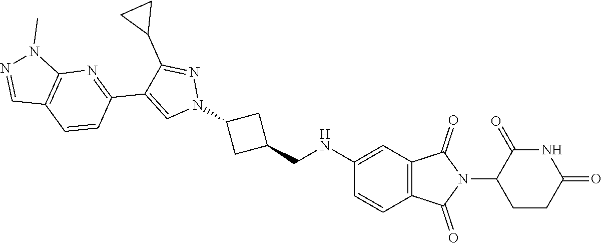 [SMILES: Cn1ncc2ccc(-c3cn([C@H]4C[C@H](CNc5ccc6c(c5)C(=O)N(C5CCC(=O)NC5=O)C6=O)C4)nc3C3CC3)nc21] | 5-(((trans-3-(3-cyclopropyl-4-(1-methyl- 1H-pyrazolo[3,4-b]pyridin-6-yl)-1H- pyrazol-1-yl)cyclobutyl)methyl)amino)- 2-(2,6-dioxopiperidin-3-yl)isoindoline- 1,3-dione |
| GS- 730 | 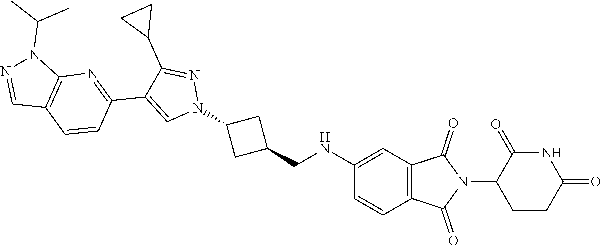 [SMILES: CC(C)n1ncc2ccc(-c3cn([C@H]4C[C@H](CNc5ccc6c(c5)C(=O)N(C5CCC(=O)NC5=O)C6=O)C4)nc3C3CC3)nc21] | 5-(((trans-3-(3-cyclopropyl-4-(1- isopropyl-1H-pyrazolo[3,4-b]pyridin-6- yl)-1 H-pyrazol-1- yl)cyclobutyl)methyl)amino)-2-(2,6- dioxopiperidin-3-yl)isoindoline-1,3- dione |
| GS- 731 | 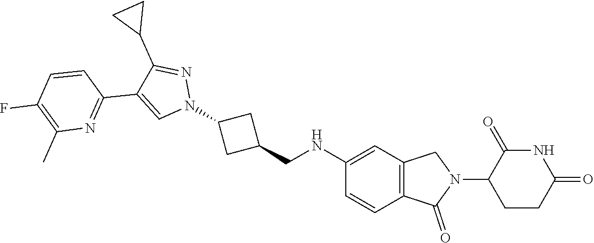 [SMILES: Cc1nc(-c2cn([C@H]3C[C@H](CNc4ccc5c(c4)CN(C4CCC(=O)NC4=O)C5=O)C3)nc2C2CC2)ccc1F] | 3-(5-(((trans-3-(3-cyclopropyl-4-(5- fluoro-6-methylpyridin-2-yl)-1H- pyrazol-1-yl)cyclobutyl)methyl)amino)- 1-oxoisoindolin-2-yl)piperidine-2,6- dione |
| GS- 732 | 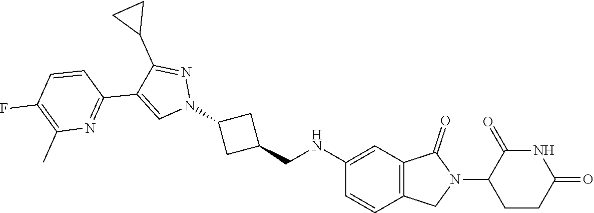 [SMILES: Cc1nc(-c2cn([C@H]3C[C@H](CNc4ccc5c(c4)C(=O)N(C4CCC(=O)NC4=O)C5)C3)nc2C2CC2)ccc1F] | 3-(6-(((trans-3-(3-cyclopropyl-4-(5- fluoro-6-methylpyridin-2-yl)-1H- pyrazol-1-yl)cyclobutyl)methyl)amino)- 1-oxoisoindolin-2-yl)piperidine-2,6- dione |
| GS- 733 | 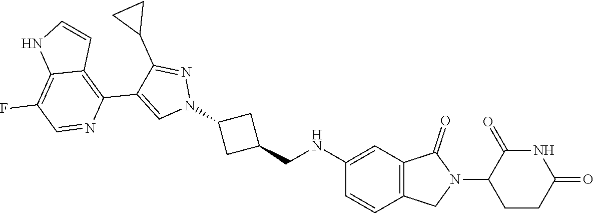 [SMILES: O=C1CCC(N2Cc3ccc(NC[C@H]4C[C@H](n5cc(-c6ncc(F)c7[nH]ccc67)c(C6CC6)n5)C4)cc3C2=O)C(=O)N1] | 5-(((trans-3-(3-cyclopropyl-4-(7-fluoro- 1H-pyrrolo[3,2-c]pyridin-4-yl)-1H- pyrazol-1-yl)cyclobutyl)methyl)amino)- 2-(2,6-dioxopiperidin-3-yl)isoindoline- 1,3-dione |
| GS- 735 | 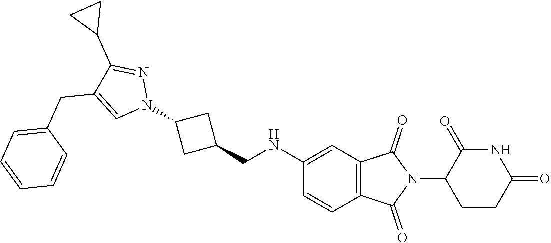 [SMILES: O=C1CCC(N2C(=O)c3ccc(NC[C@H]4C[C@H](n5cc(Cc6ccccc6)c(C6CC6)n5)C4)cc3C2=O)C(=O)N1] | 5-(((trans-3-(4-benzyl-3-cyclopropyl- 1H-pyrazol-1- yl)cyclobutyl)methyl)amino)-2-(2,6- dioxopiperidin-3-yl)isoindoline-1,3- dione |
| GS- 736 | 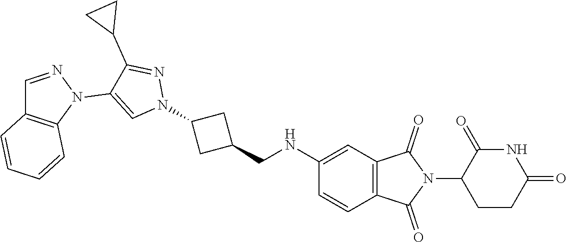 [SMILES: O=C1CCC(N2C(=O)c3ccc(NC[C@H]4C[C@H](n5cc(-n6ncc7ccccc76)c(C6CC6)n5)C4)cc3C2=O)C(=O)N1] | 5-(((trans-3-(3-cyclopropyl-4-(1H- indazol-1-yl)-1H-pyrazol-1- yl)cyclobutyl)methyl)amino)-2-(2,6- dioxopiperidin-3-yl)isoindoline-1,3- dione |
| GS- 737 | 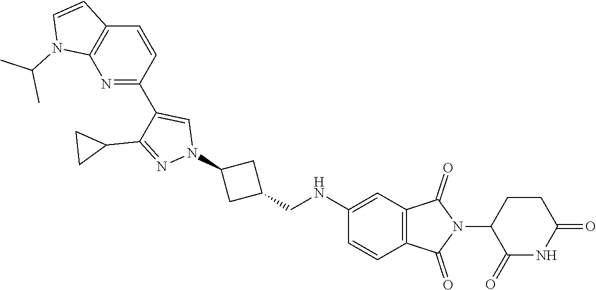 [SMILES: CC(C)n1ccc2ccc(-c3cn([C@H]4C[C@H](CNc5ccc6c(c5)C(=O)N(C5CCC(=O)NC5=O)C6=O)C4)nc3C3CC3)nc21] | 5-(((trans-3-(3-cyclopropyl-4-(1- isopropyl-1H-pyrrolo[2,3-b]pyridin-6- yl)-1 H-pyrazol-1- yl)cyclobutyl)methyl)amino)-2-(2,6- dioxopiperidin-3-yl)isoindoline-1,3- dione |
| GS- 738 | 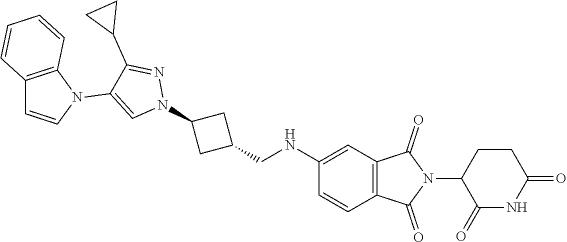 [SMILES: O=C1CCC(N2C(=O)c3ccc(NC[C@H]4C[C@H](n5cc(-n6ccc7ccccc76)c(C6CC6)n5)C4)cc3C2=O)C(=O)N1] | 5-(((trans-3-(3-cyclopropyl-4-(1H-indol- 1-yl)-1H-pyrazol-1- yl)cyclobutyl)methyl)amino)-2-(2,6- dioxopiperidin-3-yl)isoindoline-1,3- dione |
| GS- 739 | 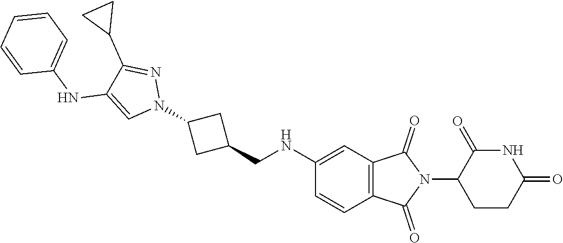 [SMILES: O=C1CCC(N2C(=O)c3ccc(NC[C@H]4C[C@H](n5cc(Nc6ccccc6)c(C6CC6)n5)C4)cc3C2=O)C(=O)N1] | 5-(((trans-3-(3-cyclopropyl-4- (phenylamino)-1 H-pyrazol-1- yl)cyclobutyl)methyl)amino)-2-(2,6- dioxopiperidin-3-yl)isoindoline-1,3- dione |
| GS- 740 | 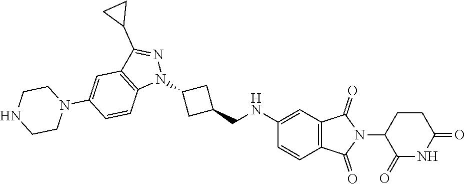 [SMILES: O=C1CCC(N2C(=O)c3ccc(NC[C@H]4C[C@H](n5nc(C6CC6)c6cc(N7CCNCC7)ccc65)C4)cc3C2=O)C(=O)N1] | 5-(((trans-3-(3-cyclopropyl-5-(piperazin- 1-yl)-1 H-indazol-1- yl)cyclobutyl)methyl)amino)-2-(2,6- dioxopiperidin-3-yl)isoindoline-1,3- dione |
| GS- 741 | 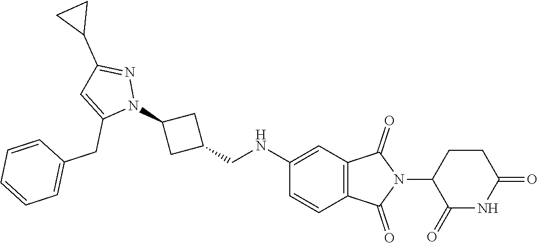 [SMILES: O=C1CCC(N2C(=O)c3ccc(NC[C@H]4C[C@H](n5nc(C6CC6)cc5Cc5ccccc5)C4)cc3C2=O)C(=O)N1] | 5-(((trans-3-(5-benzyl-3-cyclopropyl- 1H-pyrazol-1- yl)cyclobutyl)methyl)amino)-2-(2,6- dioxopiperidin-3-yl)isoindoline-1,3- dione |
| GS- 742 | 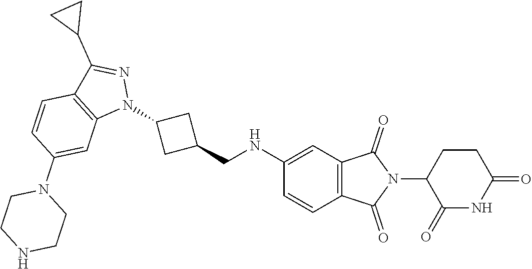 [SMILES: O=C1CCC(N2C(=O)c3ccc(NC[C@H]4C[C@H](n5nc(C6CC6)c6ccc(N7CCNCC7)cc65)C4)cc3C2=O)C(=O)N1] | 5-(((trans-3-(3-cyclopropyl-6-(piperazin- 1-yl)-1H-indazol-1- yl)cyclobutyl)methyl)amino)-2-(2,6- dioxopiperidin-3-yl)isoindoline-1,3- dione |
| GS- 743 | 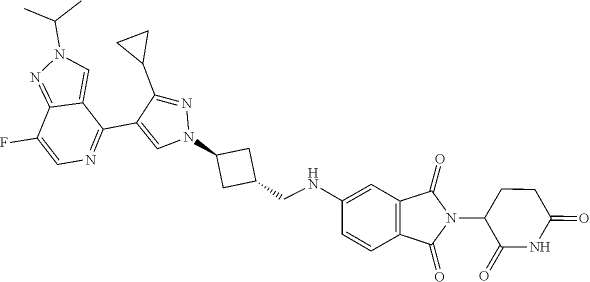 [SMILES: CC(C)n1cc2c(-c3cn([C@H]4C[C@H](CNc5ccc6c(c5)C(=O)N(C5CCC(=O)NC5=O)C6=O)C4)nc3C3CC3)ncc(F)c2n1] | 5-(((trans-3-(3-cyclopropyl-4-(7-fluoro- 2-isopropyl-2H-pyrazolo[4,3-c]pyridin- 4-yl)-1H-pyrazol-1- yl)cyclobutyl)methyl)amino)-2-(2,6- dioxopiperidin-3-yl)isoindoline-1,3- dione |
| GS- 744 | 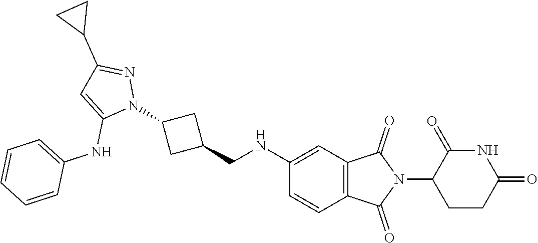 [SMILES: O=C1CCC(N2C(=O)c3ccc(NC[C@H]4C[C@H](n5nc(C6CC6)cc5Nc5ccccc5)C4)cc3C2=O)C(=O)N1] | 5-(((trans-3-(3-cyclopropyl-5- (phenylamino)-1H-pyrazol-1- yl)cyclobutyl)methyl)amino)-2-(2,6- dioxopiperidin-3-yl)isoindoline-1,3- dione |
| GS- 745 | 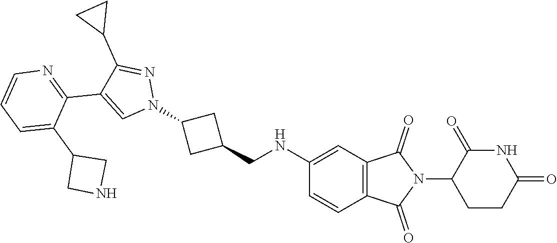 [SMILES: O=C1CCC(N2C(=O)c3ccc(NC[C@H]4C[C@H](n5cc(-c6ncccc6C6CNC6)c(C6CC6)n5)C4)cc3C2=O)C(=O)N1] | 5-(((trans-3-(4-(3-(azetidin-3-yl)pyridin- 2-yl)-3-cyclopropyl-1H-pyrazol-1- yl)cyclobutyl)methyl)amino)-2-(2,6- dioxopiperidin-3-yl)isoindoline-1,3- dione |
| GS- 746 | 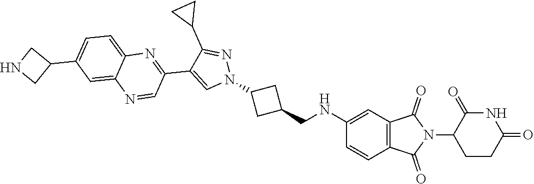 | 5-(((trans-3-(4-(6-(azetidin-3- yl)quinoxalin-2-yl)-3-cyclopropyl-1H- pyrazol-1-yl)cyclobutyl)methyl)amino)- 2-(2,6-dioxopiperidin-3-yl)isoindoline- 1,3-dione |
| GS- 747 | 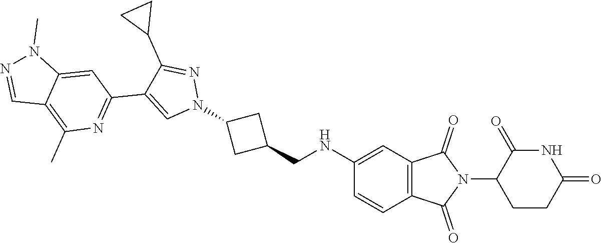 [SMILES: Cc1nc(-c2cn([C@H]3C[C@H](CNc4ccc5c(c4)C(=O)N(C4CCC(=O)NC4=O)C5=O)C3)nc2C2CC2)cc2c1cnn2C] | 5-(((trans-3-(3-cyclopropyl-4-(1,4- dimethyl-1H-pyrazolo[4,3-c]pyridin-6- yl)-1 H-pyrazol-1 - yl)cyclobutyl)methyl)amino)-2-(2,6- dioxopiperidin-3-yl)isoindoline-1,3- dione |
| GS- 748 | 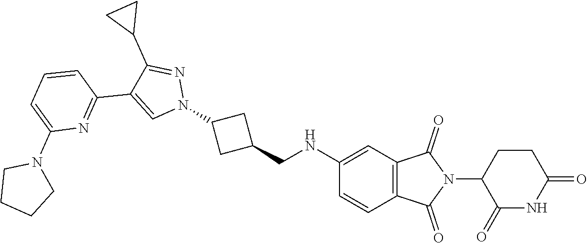 | 5-(((trans-3-(3-cyclopropyl-4-(6- (pyrrolidin-1-yl)pyridin-2-yl)-1H- pyrazol-1-yl)cyclobutyl)methyl)amino)- 2-(2,6-dioxopiperidin-3-yl)isoindoline- 1,3-dione |
| GS- 749 | 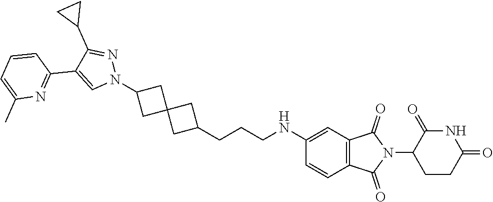 [SMILES: Cc1cccc(-c2cn(C3CC4(CC(CCCNc5ccc6c(c5)C(=O)N(C5CCC(=O)NC5=O)C6=O)C4)C3)nc2C2CC2)n1] | 5-((2-(6-(3-cyclopropyl-4-(6- methylpyridin-2-yl)-1H-pyrazol-1- yl)spiro[3.3]heptan-2-yl)ethyl)amino)-2- (2,6-dioxopiperidin-3-yl)isoindoline-1,3- dione |
| GS- 750 | 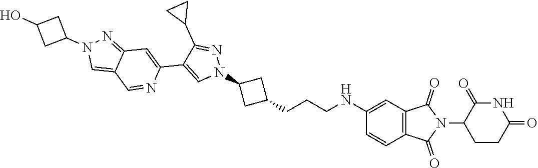 [SMILES: O=C1CCC(N2C(=O)c3ccc(NCCC[C@H]4C[C@H](n5cc(-c6cc7nn(C8CC(O)C8)cc7cn6)c(C6CC6)n5)C4)cc3C2=O)C(=O)N1] | 5-(((trans-3-(3-cyclopropyl-4-(2-(3- hydroxycyclobutyl)-2H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 751 | 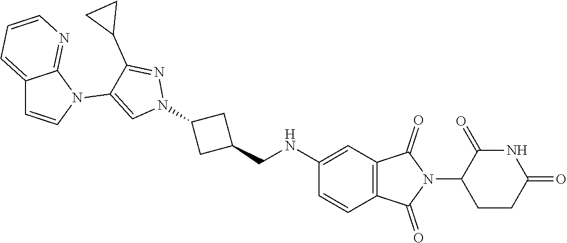 [SMILES: O=C1CCC(N2C(=O)c3ccc(NC[C@H]4C[C@H](n5cc(-n6ccc7cccnc76)c(C6CC6)n5)C4)cc3C2=O)C(=O)N1] | 5-(((trans-3-(3-cyclopropyl-4-(1H- pyrrolo[2,3-b]pyridin-1-yl)-1H-pyrazol- 1-yl)cyclobutyl)methyl)amino)-2-(2,6- dioxopiperidin-3-yl)isoindoline-1,3- dione |
| GS- 752 | 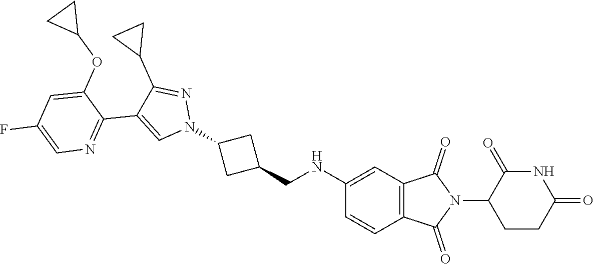 [SMILES: O=C1CCC(N2C(=O)c3ccc(NC[C@H]4C[C@H](n5cc(-c6ncc(F)cc6OC6CC6)c(C6CC6)n5)C4)cc3C2=O)C(=O)N1] | 5-(((trans-3-(4-(3-cyclopropoxy-5- fluoropyridin-2-yl)-3-cyclopropyl-1H- pyrazol-1-yl)cyclobutyl)methyl)amino)- 2-(2,6-dioxopiperidin-3-yl)isoindoline- 1,3-dione |
| GS- 753 | 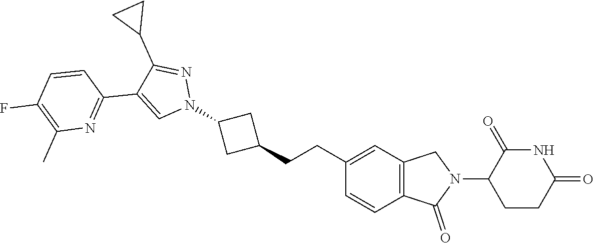 [SMILES: Cc1nc(-c2cn([C@H]3C[C@H](CCc4ccc5c(c4)CN(C4CCC(=O)NC4=O)C5=O)C3)nc2C2CC2)ccc1F] | 3-(5-(2-((1r,3s)-3-(3-cyclopropyl-4-(5- fluoro-6-methylpyridin-2-yl)-1H- pyrazol-1-yl)cyclobutyl)ethyl)-1- oxoisoindolin-2-yl)piperidine-2,6-dione |
| GS- 754 | 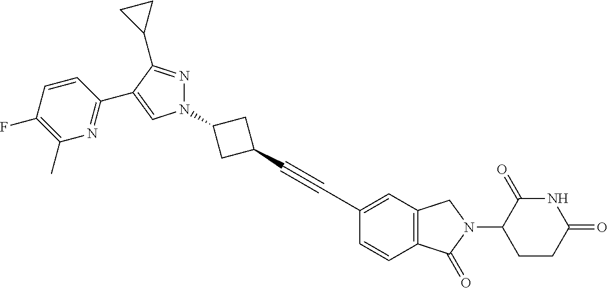 [SMILES: Cc1nc(-c2cn([C@H]3C[C@H](C#Cc4ccc5c(c4)CN(C4CCC(=O)NC4=O)C5=O)C3)nc2C2CC2)ccc1F] | 3-(5-((trans-3-(3-cyclopropyl-4-(5- fluoro-6-methylpyridin-2-yl)-1H- pyrazol-1-yl)cyclobutyl)ethynyl)-1- oxoisoindolin-2-yl)piperidine-2,6-dione |
| GS- 755 | 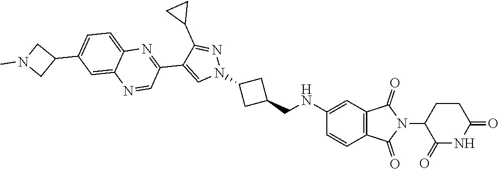 [SMILES: CN1CC(c2ccc3nc(-c4cn([C@H]5C[C@H](CNc6ccc7c(c6)C(=O)N(C6CCC(=O)NC6=O)C7=O)C5)nc4C4CC4)cnc3c2)C1] | 5-(((trans-3-(3-cyclopropyl-4-(6-(1- methylazetidin-3-yl)quinoxalin-2-yl)- 1H-pyrazol-1- yl)cyclobutyl)methyl)amino)-2-(2,6- dioxopiperidin-3-yl)isoindoline-1,3- dione |
| GS- 756 | 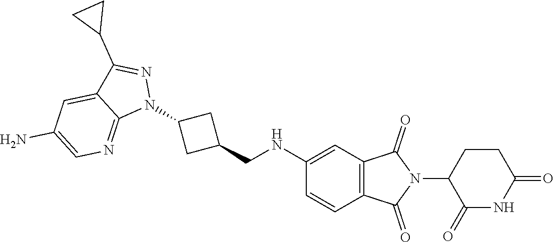 [SMILES: Nc1cnc2c(c1)c(C1CC1)nn2[C@H]1C[C@H](CNc2ccc3c(c2)C(=O)N(C2CCC(=O)NC2=O)C3=O)C1] | 5-(((trans-3-(5-amino-3-cyclopropyl-1H- pyrazolo[3,4-b]pyridin-1- yl)cyclobutyl)methyl)amino)-2-(2,6- dioxopiperidin-3-yl)isoindoline-1,3- dione |
| GS- 757 | 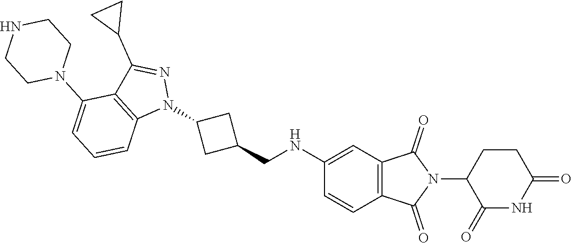 [SMILES: O=C1CCC(N2C(=O)c3ccc(NC[C@H]4C[C@H](n5nc(C6CC6)c6c(N7CCNCC7)cccc65)C4)cc3C2=O)C(=O)N1] | 5-(((trans-3-(3-cyclopropyl-4-(piperazin- 1-yl)-1 H-indazol-1- yl)cyclobutyl)methyl)amino)-2-(2,6- dioxopiperidin-3-yl)isoindoline-1,3- dione |
| GS- 758 | 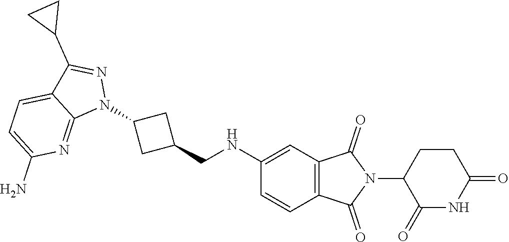 [SMILES: Nc1ccc2c(C3CC3)nn([C@H]3C[C@H](CNc4ccc5c(c4)C(=O)N(C4CCC(=O)NC4=O)C5=O)C3)c2n1] | 5-(((trans-3-(6-amino-3-cyclopropyl-1H- pyrazolo[3,4-b]pyridin-1- yl)cyclobutyl)methyl)amino)-2-(2,6- dioxopiperidin-3-yl)isoindoline-1,3- dione |
| GS- 759 | 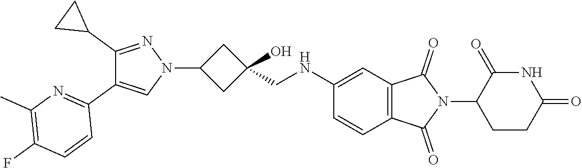 | 5-(((cis-3-(3-cyclopropyl-4-(5-fluoro-6- methylpyridin-2-yl)-1H-pyrazol-1-yl)-1- hydroxycyclobutyl)methyl)amino)-2- (2,6-dioxopiperidin-3-yl)isoindoline-1,3- dione |
| GS- 760 | 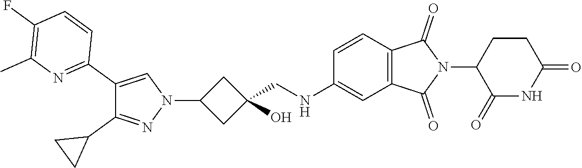 | 5-(((trans-3-(3-cyclopropyl-4-(5-fluoro- 6-methylpyridin-2-yl)-1H-pyrazol-1-yl)- 1-hydroxycyclobutyl)methyl)amino)-2- (2,6-dioxopiperidin-3-yl)isoindoline-1,3- dione |
| GS- 761 | 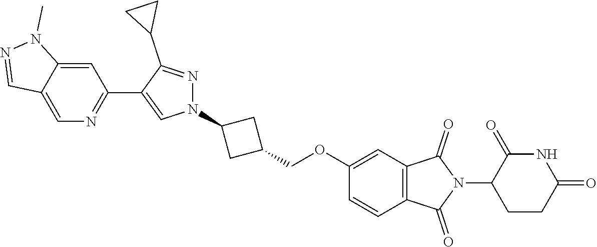 [SMILES: Cn1ncc2cnc(-c3cn([C@H]4C[C@H](COc5ccc6c(c5)C(=O)N(C5CCC(=O)NC5=O)C6=O)C4)nc3C3CC3)cc21] | 5-((trans-3-(3-cyclopropyl-4-(1-methyl- 1H-pyrazolo[4,3-c]pyridin-6-yl)-1H- pyrazol-1-yl)cyclobutyl)methoxy)-2- (2,6-dioxopiperidin-3-yl)isoindoline-1,3- dione |
| GS- 762 | 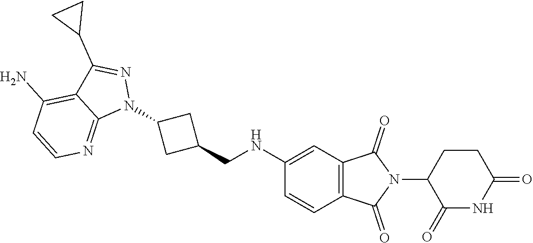 [SMILES: Nc1ccnc2c1c(C1CC1)nn2[C@H]1C[C@H](CNc2ccc3c(c2)C(=O)N(C2CCC(=O)NC2=O)C3=O)C1] | 5-(((trans-3-(4-amino-3-cyclopropyl-1H- pyrazolo[3,4-b]pyridin-1- yl)cyclobutyl)methyl)amino)-2-(2,6- dioxopiperidin-3-yl)isoindoline-1,3- dione |
| GS- 763 | 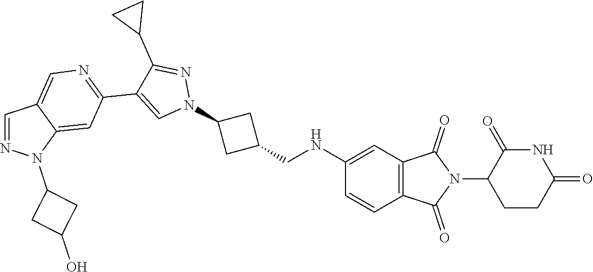 [SMILES: O=C1CCC(N2C(=O)c3ccc(NC[C@H]4C[C@H](n5cc(-c6cc7c(cn6)cnn7C6CC(O)C6)c(C6CC6)n5)C4)cc3C2=O)C(=O)N1] | 5-(((trans-3-(3-cyclopropyl-4-(1-(3- hydroxycyclobutyl)-1H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 764 | 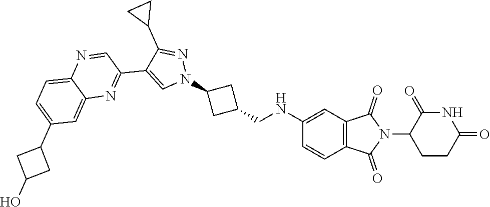 [SMILES: O=C1CCC(N2C(=O)c3ccc(NC[C@H]4C[C@H](n5cc(-c6cnc7ccc(C8CC(O)C8)cc7n6)c(C6CC6)n5)C4)cc3C2=O)C(=O)N1] | 5-(((trans-3-(3-cyclopropyl-4-(7-(3- hydroxycyclobutyl)quinoxalin-2-yl)-1H- pyrazol-1-yl)cyclobutyl)methyl)amino)- 2-(2,6-dioxopiperidin-3-yl)isoindoline- 1,3-dione |
| GS- 765 | 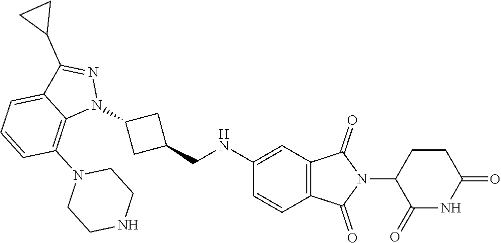 [SMILES: O=C1CCC(N2C(=O)c3ccc(NC[C@H]4C[C@H](n5nc(C6CC6)c6cccc(N7CCNCC7)c65)C4)cc3C2=O)C(=O)N1] | 5-(((trans-3-(3-cyclopropyl-7-(piperazin- 1-yl)-1 H-indazol-1- yl)cyclobutyl)methyl)amino)-2-(2,6- dioxopiperidin-3-yl)isoindoline-1,3- dione |
| GS- 766 | 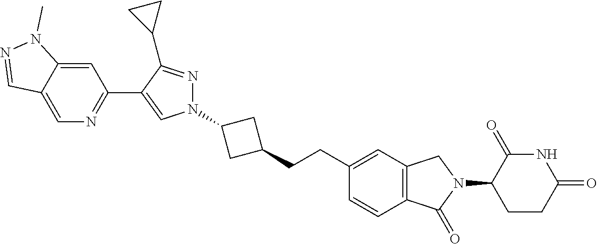 [SMILES: Cn1ncc2cnc(-c3cn([C@H]4C[C@H](CCc5ccc6c(c5)CN([C@@H]5CCC(=O)NC5=O)C6=O)C4)nc3C3CC3)cc21] | 3-(5-(2-(trans-3-(3-cyclopropyl-4-(1- methyl-1H-pyrazolo[4,3-c]pyridin-6-yl)- 1H-pyrazol-1-yl)cyclobutyl)ethyl)-1- oxoisoindolin-2-yl)piperidine-2,6-dione |
| GS- 767 | 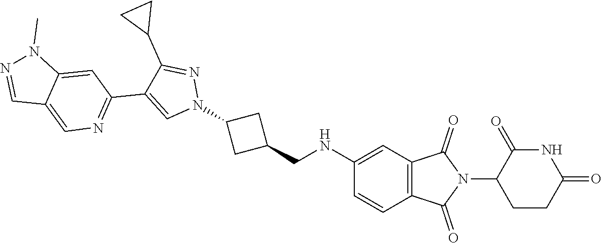 [SMILES: Cn1ncc2cnc(-c3cn([C@H]4C[C@H](CNc5ccc6c(c5)C(=O)N(C5CCC(=O)NC5=O)C6=O)C4)nc3C3CC3)cc21] | 5-(2-(trans-3-(3-cyclopropyl-4-(1- methyl-1H-pyrazolo[4,3-c]pyridin-6-yl)- 1H-pyrazol-1-yl)cyclobutyl)ethyl)-2- (2,6-dioxopiperidin-3-yl)isoindoline-1,3- dione |
| GS- 768 | 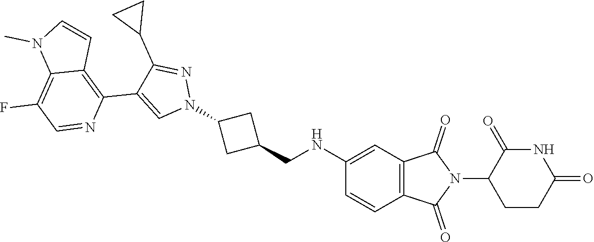 [SMILES: Cn1ccc2c(-c3cn([C@H]4C[C@H](CNc5ccc6c(c5)C(=O)N(C5CCC(=O)NC5=O)C6=O)C4)nc3C3CC3)ncc(F)c21] | 5-(((trans-3-(3-cyclopropyl-4-(7-fluoro- 1-methyl-1H-pyrrolo[3,2-c]pyridin-4-yl)- 1H-pyrazol-1- yl)cyclobutyl)methyl)amino)-2-(2,6- dioxopiperidin-3-yl)isoindoline-1,3- dione |
| GS- 769 | 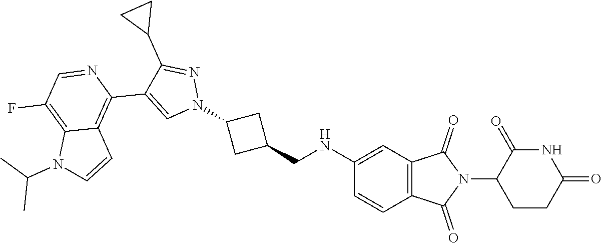 [SMILES: CC(C)n1ccc2c(-c3cn([C@H]4C[C@H](CNc5ccc6c(c5)C(=O)N(C5CCC(=O)NC5=O)C6=O)C4)nc3C3CC3)ncc(F)c21] | 5-(((trans-3-(3-cyclopropyl-4-(7-fluoro- 1-isopropyl-1H-pyrrolo[3,2-c]pyridin-4- yl)-1H-pyrazol-1- yl)cyclobutyl)methyl)amino)-2-(2,6- dioxopiperidin-3-yl)isoindoline-1,3- dione |
| GS- 770 | 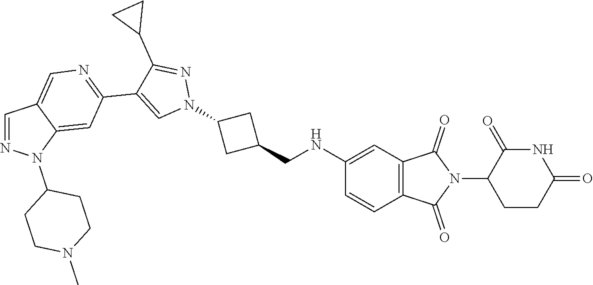 [SMILES: CN1CCC(n2ncc3cnc(-c4cn([C@H]5C[C@H](CNc6ccc7c(c6)C(=O)N(C6CCC(=O)NC6=O)C7=O)C5)nc4C4CC4)cc32)CC1] | 5-(((trans-3-(3-cyclopropyl-4-(1-(1- methylpiperidin-4-yl)-1H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 771 | 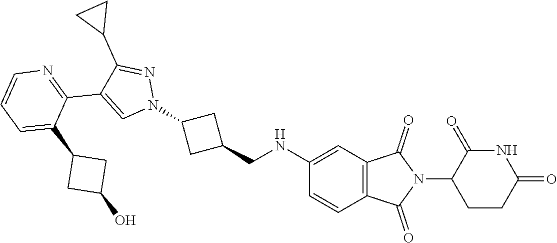 [SMILES: O=C1CCC(N2C(=O)c3ccc(NC[C@H]4C[C@H](n5cc(-c6ncccc6[C@H]6C[C@@H](O)C6)c(C6CC6)n5)C4)cc3C2=O)C(=O)N1] | 5-(((trans-3-(3-cyclopropyl-4-(3-(cis-3- hydroxycyclobutyl)pyridin-2-yl)-1H- pyrazol-1-yl)cyclobutyl)methyl)amino)- 2-(2,6-dioxopiperidin-3-yl)isoindoline- 1,3-dione |
| GS- 772 | 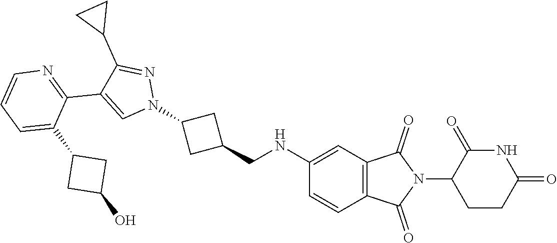 [SMILES: O=C1CCC(N2C(=O)c3ccc(NC[C@H]4C[C@H](n5cc(-c6ncccc6[C@H]6C[C@H](O)C6)c(C6CC6)n5)C4)cc3C2=O)C(=O)N1] | 5-(((trans-3-(3-cyclopropyl-4-(3-(trans- 3-hydroxycyclobutyl)pyridin-2-yl)-1H- pyrazol-1-yl)cyclobutyl)methyl)amino)- 2-(2,6-dioxopiperidin-3-yl)isoindoline- 1,3-dione |
| GS- 773 | 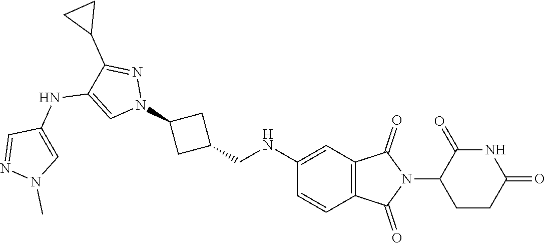 [SMILES: Cn1cc(Nc2cn([C@H]3C[C@H](CNc4ccc5c(c4)C(=O)N(C4CCC(=O)NC4=O)C5=O)C3)nc2C2CC2)cn1] | 5-(((trans-3-(3-cyclopropyl-4-((1- methyl-1H-pyrazol-4-yl)amino)-1H- pyrazol-1-yl)cyclobutyl)methyl)amino)- 2-(2,6-dioxopiperidin-3-yl)isoindoline- 1,3-dione |
| GS- 774 | 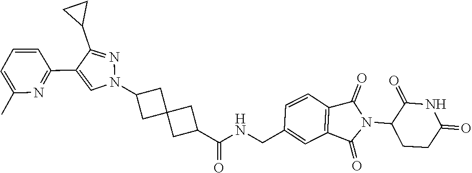 [SMILES: Cc1cccc(-c2cn(C3CC4(CC(C(=O)NCc5ccc6c(c5)C(=O)N(C5CCC(=O)NC5=O)C6=O)C4)C3)nc2C2CC2)n1] | 6-(3-cyclopropyl-4-(6-methylpyridin-2- yl)-1H-pyrazol-1-yl)-N-((2-(2,6- dioxopiperidin-3-yl)-1,3- dioxoisoindolin-5- yl)methyl)spiro[3.3]heptane-2- carboxamide |
| GS- 775 | 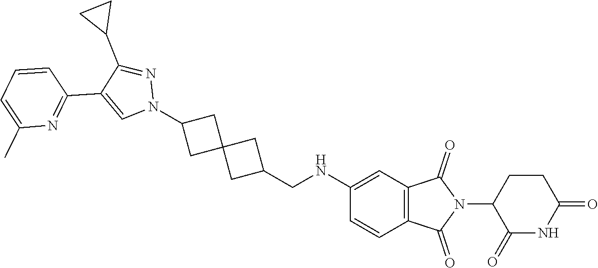 [SMILES: Cc1cccc(-c2cn(C3CC4(CC(CNc5ccc6c(c5)C(=O)N(C5CCC(=O)NC5=O)C6=O)C4)C3)nc2C2CC2)n1] | 5-(((6-(3-cyclopropyl-4-(6- methylpyridin-2-yl)-1H-pyrazol-1- yl)spiro[3.3]heptan-2-yl)methyl)amino)- 2-(2,6-dioxopiperidin-3-yl)isoindoline- 1,3-dione |
| GS- 776 | 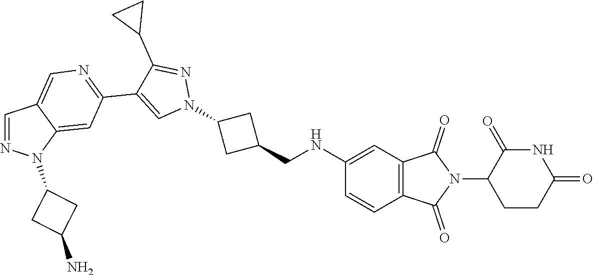 [SMILES: N[C@H]1C[C@H](n2ncc3cnc(-c4cn([C@H]5C[C@H](CNc6ccc7c(c6)C(=O)N(C6CCC(=O)NC6=O)C7=O)C5)nc4C4CC4)cc32)C1] | 5-(((trans-3-(4-(1-(trans-3- aminocyclobutyl)-1H-pyrazolo[4,3- c]pyridin-6-yl)-3-cyclopropyl-1H- pyrazol-1-yl)cyclobutyl)methyl)amino)- 2-(2,6-dioxopiperidin-3-yl)isoindoline- 1,3-dione |
| GS- 777 | 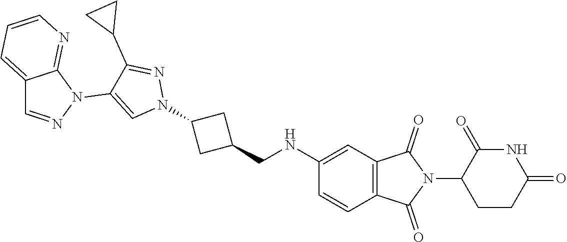 [SMILES: O=C1CCC(N2C(=O)c3ccc(NC[C@H]4C[C@H](n5cc(-n6ncc7cccnc76)c(C6CC6)n5)C4)cc3C2=O)C(=O)N1] | 5-(((trans-3-(3-cyclopropyl-4-(1H- pyrazolo[3,4-b]pyridin-1-yl)-1H-pyrazol- 1-yl)cyclobutyl)methyl)amino)-2-(2,6- dioxopiperidin-3-yl)isoindoline-1,3- dione |
| GS- 778 | 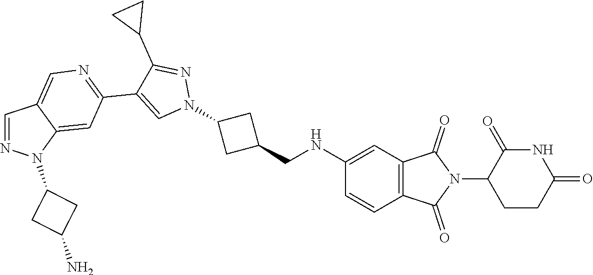 [SMILES: N[C@H]1C[C@@H](n2ncc3cnc(-c4cn([C@H]5C[C@H](CNc6ccc7c(c6)C(=O)N(C6CCC(=O)NC6=O)C7=O)C5)nc4C4CC4)cc32)C1] | 5-(((trans-3-(4-(1-(cis-3- aminocyclobutyl)-1H-pyrazolo[4,3- c]pyridin-6-yl)-3-cyclopropyl-1H- pyrazol-1-yl)cyclobutyl)methyl)amino)- 2-(2,6-dioxopiperidin-3-yl)isoindoline- 1,3-dione |
| GS- 779 | 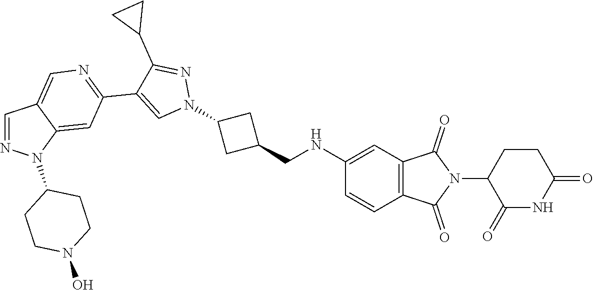 | 5-(((trans-3-(3-cyclopropyl-4-(1-(trans- 4-hydroxycyclohexyl)-1H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 780 | 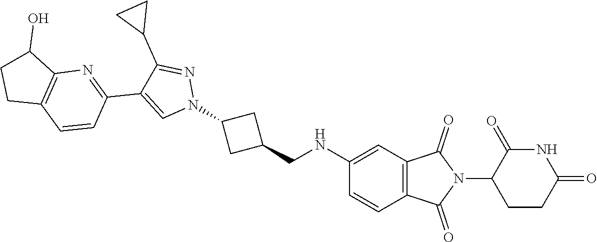 [SMILES: O=C1CCC(N2C(=O)c3ccc(NC[C@H]4C[C@H](n5cc(-c6ccc7c(n6)C(O)CC7)c(C6CC6)n5)C4)cc3C2=O)C(=O)N1] | 5-(((trans-3-(3-cyclopropyl-4-(7- hydroxy-6,7-dihydro-5H- cyclopenta[b]pyridin-2-yl)-1H-pyrazol- 1-yl)cyclobutyl)methyl)amino)-2-(2,6- dioxopiperidin-3-yl)isoindoline-1,3- dione |
| GS- 781 | 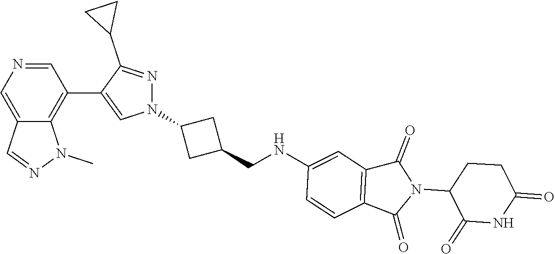 [SMILES: Cn1ncc2cncc(-c3cn([C@H]4C[C@H](CNc5ccc6c(c5)C(=O)N(C5CCC(=O)NC5=O)C6=O)C4)nc3C3CC3)c21] | 5-(((trans-3-(3-cyclopropyl-4-(1-methyl- 1H-pyrazolo[4,3-c]pyridin-7-yl)-1H- pyrazol-1-yl)cyclobutyl)methyl)amino)- 2-(2,6-dioxopiperidin-3-yl)isoindoline- 1,3-dione |
| GS- 782 | 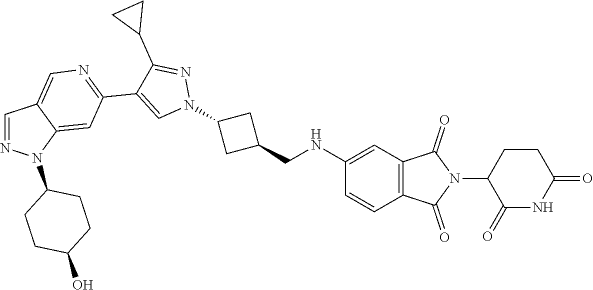 [SMILES: O=C1CCC(N2C(=O)c3ccc(NC[C@H]4C[C@H](n5cc(-c6cc7c(cn6)cnn7[C@H]6CC[C@@H](O)CC6)c(C6CC6)n5)C4)cc3C2=O)C(=O)N1] | 5-(((trans-3-(3-cyclopropyl-4-(1-(cis-4- hydroxycyclohexyl)-1H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 783 | 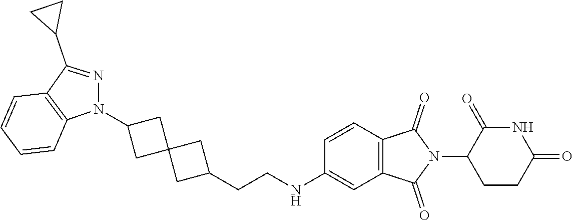 [SMILES: O=C1CCC(N2C(=O)c3ccc(NCCC4CC5(C4)CC(n4nc(C6CC6)c6ccccc64)C5)cc3C2=O)C(=O)N1] | 5-((2-(6-(3-cyclopropyl-1H-indazol-1- yl)spiro[3.3]heptan-2-yl)ethyl)amino)-2- (2,6-dioxopiperidin-3-yl)isoindoline-1,3- dione |
| GS- 784 | 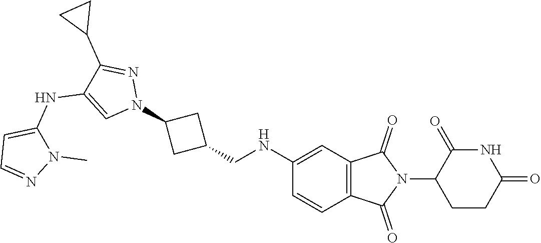 [SMILES: Cn1nccc1Nc1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-((1- methyl-1H-pyrazol-5-yl)amino)-1H- pyrazol-1-yl)cyclobutyl)methyl)amino)- 2-(2,6-dioxopiperidin-3-yl)isoindoline- 1,3-dione |
| GS- 785 | 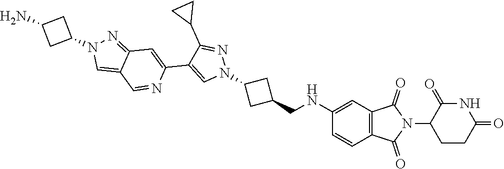 [SMILES: N[C@H]1C[C@@H](n2cc3cnc(-c4cn([C@H]5C[C@H](CNc6ccc7c(c6)C(=O)N(C6CCC(=O)NC6=O)C7=O)C5)nc4C4CC4)cc3n2)C1] | 5-(((trans-3-(4-(2-(cis-3- aminocyclobutyl)-2H-pyrazolo[4,3- c]pyridin-6-yl)-3-cyclopropyl-1H- pyrazol-1-yl)cyclobutyl)methyl)amino)- 2-(2,6-dioxopiperidin-3-yl)isoindoline- 1,3-dione |
| GS- 786 | 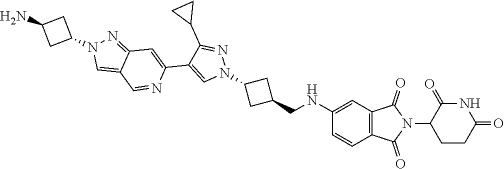 [SMILES: N[C@H]1C[C@H](n2cc3cnc(-c4cn([C@H]5C[C@H](CNc6ccc7c(c6)C(=O)N(C6CCC(=O)NC6=O)C7=O)C5)nc4C4CC4)cc3n2)C1] | 5-(((trans-3-(4-(2-(trans-3- aminocyclobutyl)-2H-pyrazolo[4,3- c]pyridin-6-yl)-3-cyclopropyl-1H- pyrazol-1-yl)cyclobutyl)methyl)amino)- 2-(2,6-dioxopiperidin-3-yl)isoindoline- 1,3-dione |
| GS- 787 | 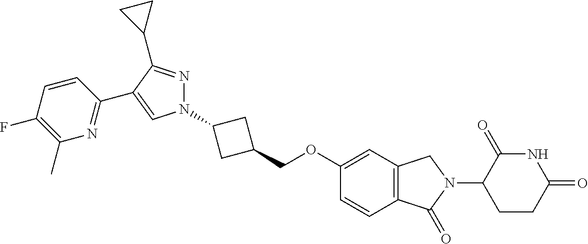 [SMILES: Cc1nc(-c2cn([C@H]3C[C@H](COc4ccc5c(c4)CN(C4CCC(=O)NC4=O)C5=O)C3)nc2C2CC2)ccc1F] | 3-(5-((trans-3-(3-cyclopropyl-4-(5- fluoro-6-methylpyridin-2-yl)-1H- pyrazol-1-yl)cyclobutyl)methoxy)-1- oxoisoindolin-2-yl)piperidine-2,6-dione |
| GS- 788 | 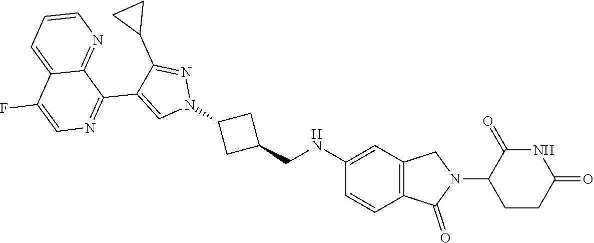 [SMILES: O=C1CCC(N2Cc3cc(NC[C@H]4C[C@H](n5cc(-c6ncc(F)c7cccnc67)c(C6CC6)n5)C4)ccc3C2=O)C(=O)N1] | 3-(5-(((trans-3-(3-cyclopropyl-4-(5- fluoro-1,7-naphthyridin-8-yl)-1H- pyrazol-1-yl)cyclobutyl)methyl)amino)- 1-oxoisoindolin-2-yl)piperidine-2,6- dione |
| GS- 789 | 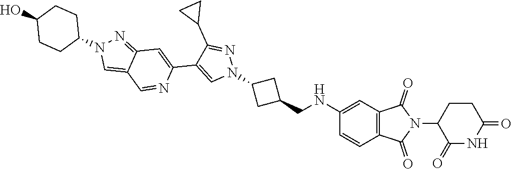 [SMILES: O=C1CCC(N2C(=O)c3ccc(NC[C@H]4C[C@H](n5cc(-c6cc7nn([C@H]8CC[C@H](O)CC8)cc7cn6)c(C6CC6)n5)C4)cc3C2=O)C(=O)N1] | 5-(((trans-3-(3-cyclopropyl-4-(2-(trans- 4-hydroxycyclohexyl)-2H-pyrazolo[4,3- c]pyridin-6-yl)-1H-pyrazol-1- yl)cyclobutyl)methyl)amino)-2-(2,6- dioxopiperidin-3-yl)isoindoline-1,3- dione |
| GS- 790 | 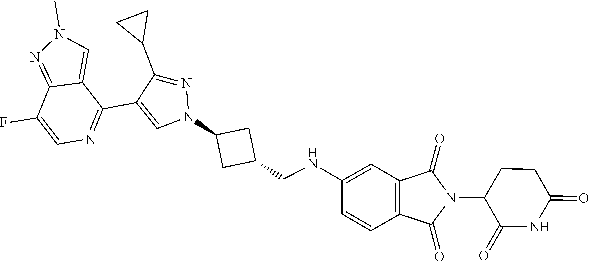 [SMILES: Cn1cc2c(-c3cn([C@H]4C[C@H](CNc5ccc6c(c5)C(=O)N(C5CCC(=O)NC5=O)C6=O)C4)nc3C3CC3)ncc(F)c2n1] | 5-(((trans-3-(3-cyclopropyl-4-(7-fluoro- 2-methyl-2H-pyrazolo[4,3-c]pyridin-4- yl)-1H-pyrazol-1- yl)cyclobutyl)methyl)amino)-2-(2,6- dioxopiperidin-3-yl)isoindoline-1,3- dione |
| GS- 791 | 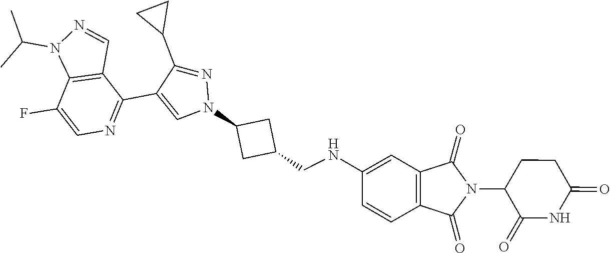 [SMILES: CC(C)n1ncc2c(-c3cn([C@H]4C[C@H](CNc5ccc6c(c5)C(=O)N(C5CCC(=O)NC5=O)C6=O)C4)nc3C3CC3)ncc(F)c21] | 5-(((trans-3-(3-cyclopropyl-4-(7-fluoro- 1-isopropyl-1H-pyrazolo[4,3-c]pyridin- 4-yl)-1H-pyrazol-1- yl)cyclobutyl)methyl)amino)-2-(2,6- dioxopiperidin-3-yl)isoindoline-1,3- dione |
| GS- 792 | 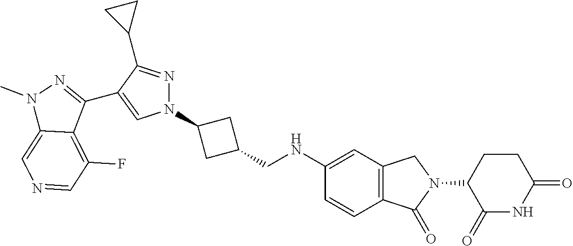 | 3-(5-(((trans-3-(3-cyclopropyl-4-(4- fluoro-1-methyl-1H-pyrazolo[3,4- c]pyridin-3-yl)-1H-pyrazol-1- yl)cyclobutyl)methyl)amino)-1- oxoisoindolin-2-yl)piperidine-2,6-dione |
| GS- 793 | 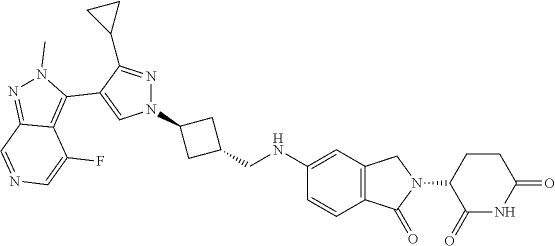 | 3-(5-(((trans-3-(3-cyclopropyl-4-(4- fluoro-2-methyl-2H-pyrazolo[3,4- c]pyridin-3-yl)-1H-pyrazol-1- yl)cyclobutyl)methyl)amino)-1- oxoisoindolin-2-yl)piperidine-2,6-dione |
| GS- 794 | 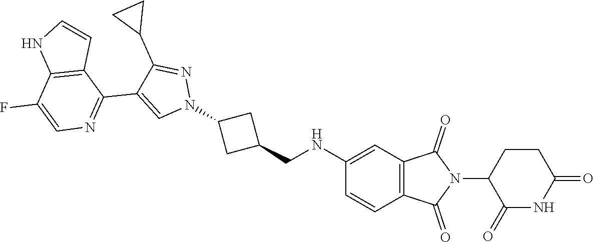 [SMILES: O=C1CCC(N2C(=O)c3ccc(NC[C@H]4C[C@H](n5cc(-c6ncc(F)c7[nH]ccc67)c(C6CC6)n5)C4)cc3C2=O)C(=O)N1] | 3-(5-(((trans-3-(3-cyclopropyl-4-(7- fluoro-1H-pyrrolo[3,2-c]pyridin-4-yl)- 1H-pyrazol-1- yl)cyclobutyl)methyl)amino)-1- oxoisoindolin-2-yl)piperidine-2,6-dione |
| GS- 795 | 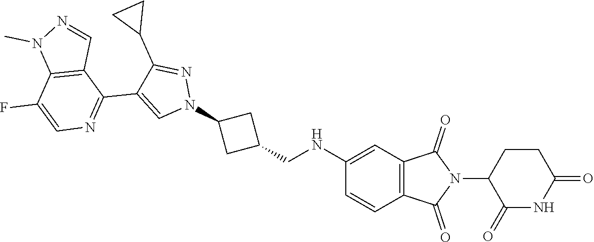 [SMILES: Cn1ncc2c(-c3cn([C@H]4C[C@H](CNc5ccc6c(c5)C(=O)N(C5CCC(=O)NC5=O)C6=O)C4)nc3C3CC3)ncc(F)c21] | 5-(((trans-3-(3-cyclopropyl-4-(7-fluoro- 1-methyl-1H-pyrazolo[4,3-c]pyridin-4- yl)-1 H-pyrazol-1- yl)cyclobutyl)methyl)amino)-2-(2,6- dioxopiperidin-3-yl)isoindoline-1,3- dione |
| GS- 796 | 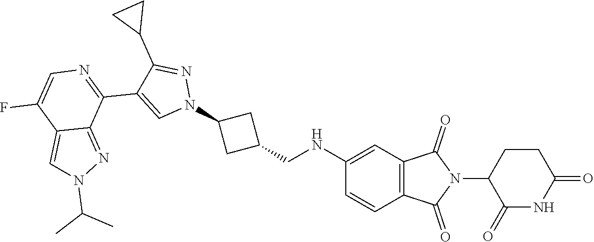 [SMILES: CC(C)n1cc2c(F)cnc(-c3cn([C@H]4C[C@H](CNc5ccc6c(c5)C(=O)N(C5CCC(=O)NC5=O)C6=O)C4)nc3C3CC3)c2n1] | 5-(((trans-3-(3-cyclopropyl-4-(4-fluoro- 2-isopropyl-2H-pyrazolo[3,4-c]pyridin- 7-yl)-1 H-pyrazol-1- yl)cyclobutyl)methyl)amino)-2-(2,6- dioxopiperidin-3-yl)isoindoline-1,3- dione |
| GS- 797 | 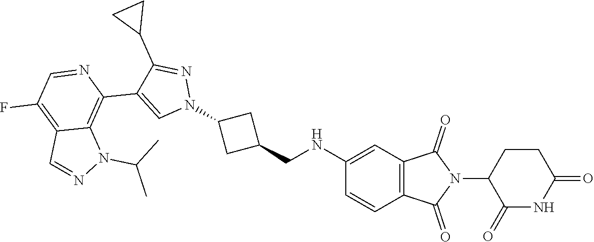 [SMILES: CC(C)n1ncc2c(F)cnc(-c3cn([C@H]4C[C@H](CNc5ccc6c(c5)C(=O)N(C5CCC(=O)NC5=O)C6=O)C4)nc3C3CC3)c21] | 5-(((trans-3-(3-cyclopropyl-4-(4-fluoro- 1-isopropyl-1H-pyrazolo[3,4-c]pyridin- 7-yl)-1 H-pyrazol-1- yl)cyclobutyl)methyl)amino)-2-(2,6- dioxopiperidin-3-yl)isoindoline-1,3- dione |
| GS- 798 | 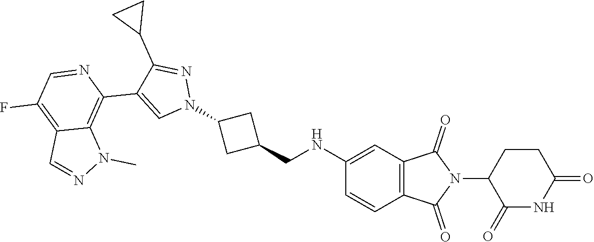 [SMILES: Cn1ncc2c(F)cnc(-c3cn([C@H]4C[C@H](CNc5ccc6c(c5)C(=O)N(C5CCC(=O)NC5=O)C6=O)C4)nc3C3CC3)c21] | 5-(((trans-3-(3-cyclopropyl-4-(4-fluoro- 1-methyl-1H-pyrazolo[3,4-c]pyridin-7- yl)-1 H-pyrazol-1- yl)cyclobutyl)methyl)amino)-2-(2,6- dioxopiperidin-3-yl)isoindoline-1,3- dione |
| GS- 799 | 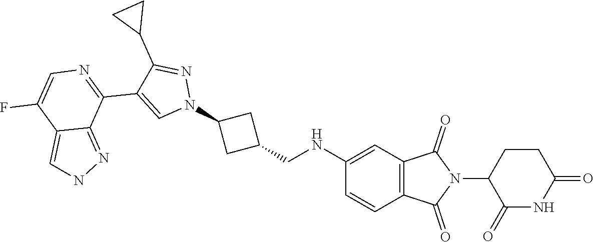 [SMILES: O=C1CCC(N2C(=O)c3ccc(NC[C@H]4C[C@H](n5cc(-c6ncc(F)c7c[nH]nc67)c(C6CC6)n5)C4)cc3C2=O)C(=O)N1] | 5-(((trans-3-(3-cyclopropyl-4-(4-fluoro- 2-methyl-2H-pyrazolo[3,4-c]pyridin-7- yl)-1H-pyrazol-1- yl)cyclobutyl)methyl)amino)-2-(2,6- dioxopiperidin-3-yl)isoindoline-1,3- dione |
| GS- 800 | 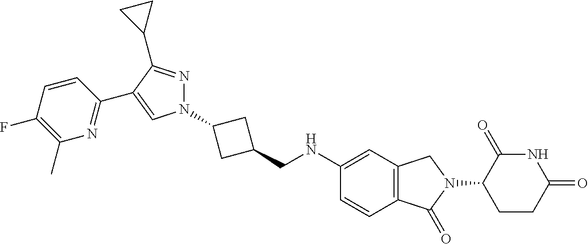 [SMILES: Cc1nc(-c2cn([C@H]3C[C@H](CNc4ccc5c(c4)CN([C@H]4CCC(=O)NC4=O)C5=O)C3)nc2C2CC2)ccc1F] | (S)-3-(5-(((trans-3-(3-cyclopropyl-4-(5- fluoro-6-methylpyridin-2-yl)-1H- pyrazol-1-yl)cyclobutyl)methyl)amino)- 1-oxoisoindolin-2-yl)piperidine-2,6- dione |
| GS- 801 | 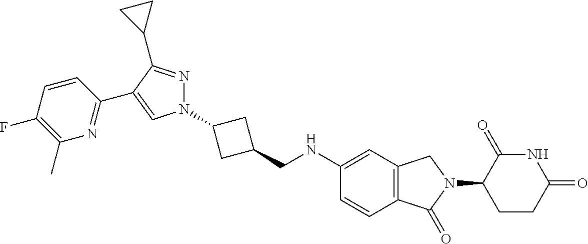 [SMILES: Cc1nc(-c2cn([C@H]3C[C@H](CNc4ccc5c(c4)CN([C@@H]4CCC(=O)NC4=O)C5=O)C3)nc2C2CC2)ccc1F] | (R)-3-(5-(((trans-3-(3-cyclopropyl-4-(5- fluoro-6-methylpyridin-2-yl)-1H- pyrazol-1-yl)cyclobutyl)methyl)amino)- 1-oxoisoindolin-2-yl)piperidine-2,6- dione |
| GS- 802 | 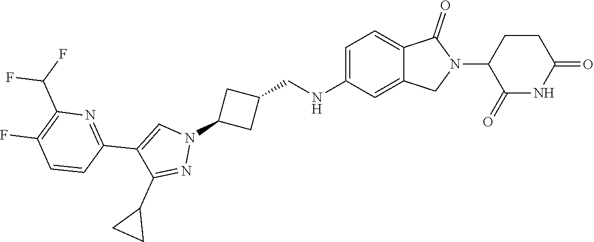 [SMILES: O=C1CCC(N2Cc3cc(NC[C@H]4C[C@H](n5cc(-c6ccc(F)c(C(F)F)n6)c(C6CC6)n5)C4)ccc3C2=O)C(=O)N1] | 3-(5-(((trans-3-(3-cyclopropyl-4-(6- (difluoromethyl)-5-fluoropyridin-2-yl)- 1H-pyrazol-1- yl)cyclobutyl)methyl)amino)-1- oxoisoindolin-2-yl)piperidine-2,6-dione |
| GS- 803 | 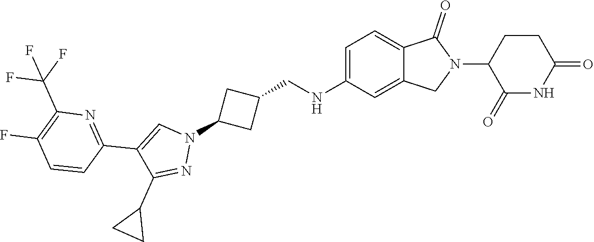 [SMILES: O=C1CCC(N2Cc3cc(NC[C@H]4C[C@H](n5cc(-c6ccc(F)c(C(F)(F)F)n6)c(C6CC6)n5)C4)ccc3C2=O)C(=O)N1] | 3-(5-(((trans-3-(3-cyclopropyl-4-(5- fluoro-6-(trifluoromethyl)pyridin-2-yl)- 1H-pyrazol-1- yl)cyclobutyl)methyl)amino)-1- oxoisoindolin-2-yl)piperidine-2,6-dione |
| GS- 804 | 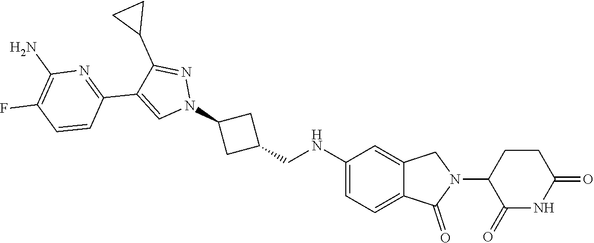 [SMILES: Nc1nc(-c2cn([C@H]3C[C@H](CNc4ccc5c(c4)CN(C4CCC(=O)NC4=O)C5=O)C3)nc2C2CC2)ccc1F] | 3-(5-(((trans-3-(4-(6-amino-5- fluoropyridin-2-yl)-3-cyclopropyl-1H- pyrazol-1-yl)cyclobutyl)methyl)amino)- 1-oxoisoindolin-2-yl)piperidine-2,6- dione |
| GS- 805 | 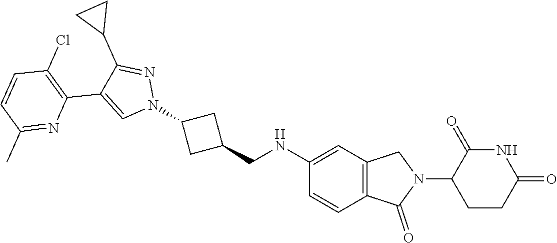 [SMILES: Cc1ccc(Cl)c(-c2cn([C@H]3C[C@H](CNc4ccc5c(c4)CN(C4CCC(=O)NC4=O)C5=O)C3)nc2C2CC2)n1] | 3-(5-(((trans-3-(4-(3-chloro-6- methylpyridin-2-yl)-3-cyclopropyl-1H- pyrazol-1-yl)cyclobutyl)methyl)amino)- 1-oxoisoindolin-2-yl)piperidine-2,6- dione |
| GS- 806 | 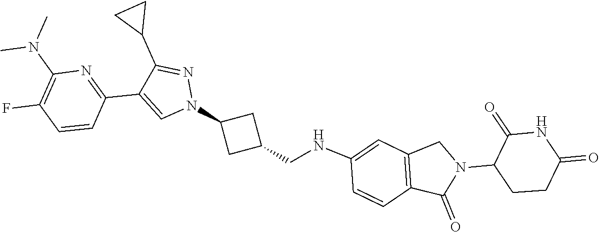 [SMILES: CN(C)c1nc(-c2cn([C@H]3C[C@H](CNc4ccc5c(c4)CN(C4CCC(=O)NC4=O)C5=O)C3)nc2C2CC2)ccc1F] | 3-(5-(((trans-3-(3-cyclopropyl-4-(6- (dimethylamino)-5-fluoropyridin-2-yl)- 1H-pyrazol-1- yl)cyclobutyl)methyl)amino)-1- oxoisoindolin-2-yl)piperidine-2,6-dione |
| GS- 807 | 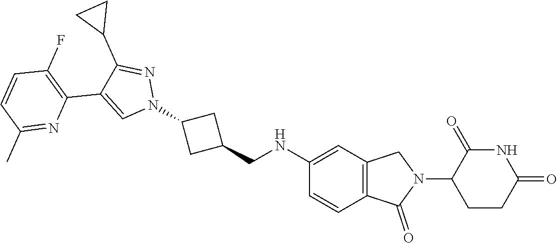 [SMILES: Cc1ccc(F)c(-c2cn([C@H]3C[C@H](CNc4ccc5c(c4)CN(C4CCC(=O)NC4=O)C5=O)C3)nc2C2CC2)n1] | 3-(5-(((trans-3-(3-cyclopropyl-4-(3- fluoro-6-methylpyridin-2-yl)-1H- pyrazol-1-yl)cyclobutyl)methyl)amino)- 1-oxoisoindolin-2-yl)piperidine-2,6- dione |
| GS- 808 | 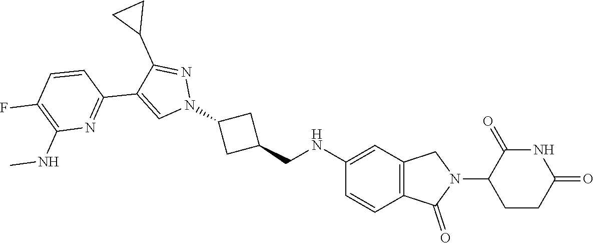 [SMILES: CNc1nc(-c2cn([C@H]3C[C@H](CNc4ccc5c(c4)CN(C4CCC(=O)NC4=O)C5=O)C3)nc2C2CC2)ccc1F] | 3-(5-(((trans-3-(3-cyclopropyl-4-(5- fluoro-6-(methylamino)pyridin-2-yl)-1H- pyrazol-1-yl)cyclobutyl)methyl)amino)- 1-oxoisoindolin-2-yl)piperidine-2,6- dione |
| GS- 809 | 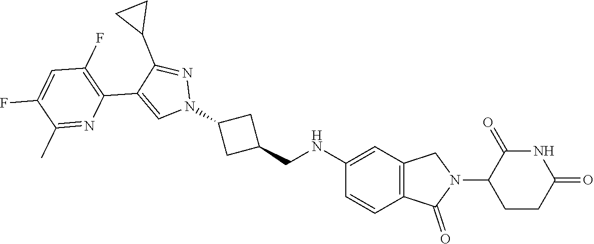 [SMILES: Cc1nc(-c2cn([C@H]3C[C@H](CNc4ccc5c(c4)CN(C4CCC(=O)NC4=O)C5=O)C3)nc2C2CC2)c(F)cc1F] | 3-(5-(((trans-3-(3-cyclopropyl-4-(3,5- difluoro-6-methylpyridin-2-yl)-1H- pyrazol-1-yl)cyclobutyl)methyl)amino)- 1-oxoisoindolin-2-yl)piperidine-2,6- dione |
| GS- 810 | 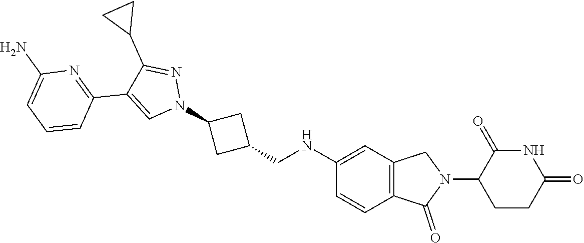 [SMILES: Nc1cccc(-c2cn([C@H]3C[C@H](CNc4ccc5c(c4)CN(C4CCC(=O)NC4=O)C5=O)C3)nc2C2CC2)n1] | 3-(5-(((trans-3-(4-(6-aminopyridin-2-yl)- 3-cyclopropyl-1H-pyrazol-1- yl)cyclobutyl)methyl)amino)-1- oxoisoindolin-2-yl)piperidine-2,6-dione |
| GS- 811 | 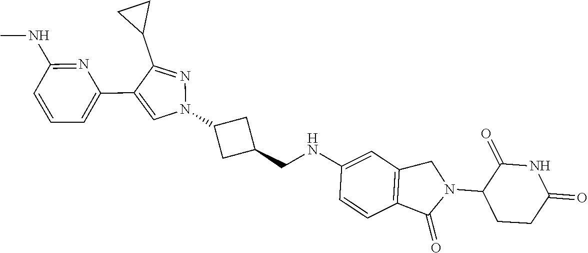 | 3-(5-(((trans-3-(3-cyclopropyl-4-(6- (methylamino)pyridin-2-yl)-1H-pyrazol- 1-yl)cyclobutyl)methyl)amino)-1- oxoisoindolin-2-yl)piperidine-2,6-dione |
| GS- 812 | 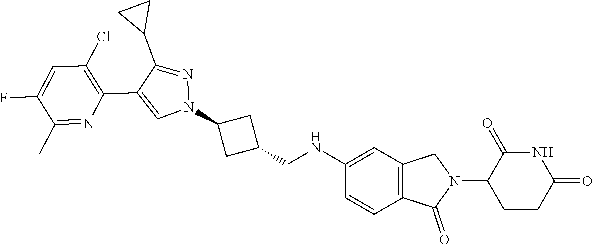 [SMILES: Cc1nc(-c2cn([C@H]3C[C@H](CNc4ccc5c(c4)CN(C4CCC(=O)NC4=O)C5=O)C3)nc2C2CC2)c(Cl)cc1F] | 3-(5-(((trans-3-(4-(3-chloro-5-fluoro-6- methylpyridin-2-yl)-3-cyclopropyl-1H- pyrazol-1-yl)cyclobutyl)methyl)amino)- 1-oxoisoindolin-2-yl)piperidine-2,6- dione |
| GS- 813 | 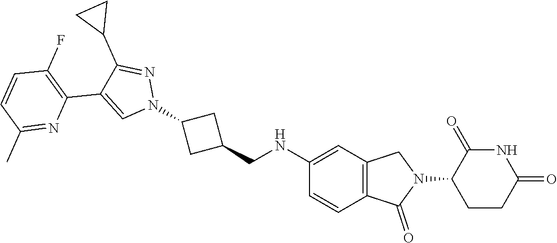 [SMILES: Cc1ccc(F)c(-c2cn([C@H]3C[C@H](CNc4ccc5c(c4)CN([C@H]4CCC(=O)NC4=O)C5=O)C3)nc2C2CC2)n1] | (S)-3-(5-(((trans-3-(3-cyclopropyl-4-(3- fluoro-6-methylpyridin-2-yl)-1H- pyrazol-1-yl)cyclobutyl)methyl)amino)- 1-oxoisoindolin-2-yl)piperidine-2,6- dione |
| GS- 814 | 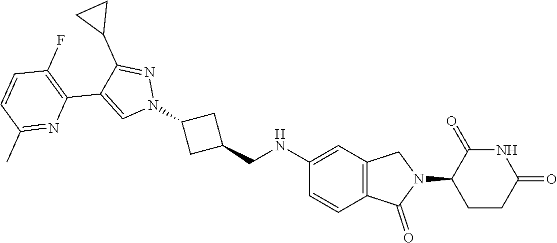 [SMILES: Cc1ccc(F)c(-c2cn([C@H]3C[C@H](CNc4ccc5c(c4)CN([C@@H]4CCC(=O)NC4=O)C5=O)C3)nc2C2CC2)n1] | (R)-3-(5-(((trans-3-(3-cyclopropyl-4-(3- fluoro-6-methylpyridin-2-yl)-1H- pyrazol-1-yl)cyclobutyl)methyl)amino)- 1-oxoisoindolin-2-yl)piperidine-2,6- dione |

### II. Synthesis and Further Forms of the Compounds

By way of non-limiting example, detailed synthesis protocols are described in the Examples for specific exemplary compounds.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof. This disclosure includes pharmaceutically acceptable salts of the structures depicted and compounds named herein.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements.

In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

In some aspects, provided herein is a method for identifying a compound which mediates degradation or reduction of GSPT1, the method comprising: providing a compound comprising an a CRBN binder; contacting the compound with a cell comprising a ubiquitin ligase and GSPT1; determining whether GSPT1 level is decreased in the cell; and identifying the compound as a compound which mediates degradation or reduction of GSPT1. In certain embodiments, the cell is a cancer cell. In certain embodiments, the cancer cell is a GSPT1-mediated cancer cell.

### III. Characterization of Exemplary Compounds

The binding affinity of the compounds disclosed herein or pharmaceutically acceptable salts thereof can be assessed using standard biophysical assays known in the art (e.g., isothermal titration calorimetry (ITC), surface plasmon resonance (SPR)). Cellular assays can then be used to assess the compound's ability to induce GSPT1 degradation and inhibit cancer cell proliferation. Besides evaluating a compound's induced changes in the protein levels of GSPT1. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include HEL, RS4;11, MV4;11, MOLT-4, CCRF-CEM, Kasumi-1, MM.1S, HL-60, WSU-DLCL2, Pfeiffer, and SU-DHL-1 cancer cell lines. Suitable mouse models for use in any or all of these steps are known in the art and include subcutaneous xenograft models, orthotopic models, patient-derived xenograft models, and patient-derived orthotopic models.

Specific exemplary compounds were characterized using cell viability assays. In the assays, MV4;11 and MOLM-13 cells were treated with compounds for three days. The IC₅₀ values ranged from 1 nM to over 10 μM.

In some embodiments, the compounds disclose herein or pharmaceutically acceptable salts thereof inhibit growth of a cell with an IC₅₀ value below 50 nM. In some embodiments, the compounds inhibit growth of a cell with an IC₅₀ value below 25 nM. In some embodiments, the compounds inhibit growth of a cell with an IC₅₀ value below 10 nM. In some embodiments, the compounds inhibit growth of a cell with an IC₅₀ value below 8 nM. In some embodiments, the compounds inhibit growth of a cell with an IC₅₀ value below 2 nM. In some embodiments, the cell comprises a MV4;11, or MOLM-13 cell. In some embodiments the cell is MV4;11 cell In some embodiments, the cell is MOLM-13 cell.

### IV. Pharmaceutical Compositions

In some aspects, the compositions and methods described herein include the manufacture and use of pharmaceutical compositions and medicaments that include one or more compounds (e.g. heterobifunctional compounds) as disclosed herein. Also included are the pharmaceutical compositions themselves. In some embodiments, the pharmaceutical composition comprises a compound described herein or a pharmaceutically acceptable salt thereof and a pharmaceutically acceptable excipient. In some embodiments, the pharmaceutical composition includes a compound described herein such as a GSPT1 degrader. In some embodiments, the pharmaceutical composition includes a second compound such as an FLT3 pathway inhibitor, a RAS-RAF-MEK-ERK pathway inhibitor, or a PI3K-AKT-mTOR pathway inhibitor or activator. The second compound may be an FLT3 pathway inhibitor or an FLT3 inhibitor such as Gilteritinib.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a subject, together with a compound of the disclosure, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this disclosure include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer).

In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some embodiments, the disclosure relates to pharmaceutical formulation comprising: (a) a therapeutically effective amount of a compound of described herein (e.g. Formula (I)) or a pharmaceutically acceptable salt thereof; and (b) a second therapeutic agent.

In some embodiments, the disclosure relates to pharmaceutical formulation comprising: (a) a therapeutically effective amount of a compound of described herein (e.g. Formula (I)) or a pharmaceutically acceptable salt thereof; and (b) one or more therapeutic agent(s).

In some embodiments, the disclosure relates to a pharmaceutical formulation comprising a therapeutically effective amount of: (a) a compound of FORMULA 1, or a pharmaceutically acceptable salt, hydrate, solvate, prodrug, stereoisomer, or tautomer thereof; and (b) one or more therapeutic agent(s).

In some embodiments, the second or more therapeutic agent(s) is/are an anti-cancer drug. In some embodiments, the second or more therapeutic agent(s) is/are an anti-proliferative agent. In some embodiments, the second or more therapeutic agent(s) is/are an immunomodulatory agent. In some embodiments, the second or more therapeutic agent(s) is a kinase inhibitor or activator. In some embodiments, the second or more therapeutic agent(s) is a kinase inhibitor.

In some embodiments, the second or more therapeutic agent(s) inhibits a FMS-like tyrosine kinase 3 gene (FLT3) pathway. In some embodiments, the second or more therapeutic agent(s) inhibits FLT3. In some embodiments, the second compound is an FLT3 pathway inhibitor. The second compound may be Gilteritinib.

In some embodiments, the second or more therapeutic agent(s) inhibits PI3K/AKT/mTOR pathway. In some embodiments, the second or more therapeutic agent(s) inhibits PI3K. In some embodiments, the second or more therapeutic agent(s) inhibits AKT. In some embodiments, the second or more therapeutic agent(s) inhibits mTOR. In some embodiments, the second compound is a PI3K-AKT-mTOR pathway inhibitor or activator.

In some embodiments, the second or more therapeutic agent(s) inhibits MAPK pathway. In some embodiments, the second or more therapeutic agent(s) inhibits RAS/RAF/MEK/ERK pathway. In some embodiments, the second or more therapeutic agent(s) inhibits RAS. In some embodiments, the second or more therapeutic agent(s) inhibits RAF. In some embodiments, the second or more therapeutic agent(s) inhibits MEK. In some embodiments, the second or more therapeutic agent(s) inhibits ERK. In some embodiments, the second compound is a RAS-RAF-MEK-ERK pathway inhibitor.

### V. Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at www.fda.gov/Drugs/DevelopmentApprovalProcess/FormsSubmissionRequirements/ElectronicSubmissio ns/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

The pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

In some embodiments, the pharmaceutical compositions of this disclosure are orally administered in any orally acceptable dosage form including, but not limited to, capsules, tablets, emulsions and aqueous suspensions, dispersions and solutions.

In some embodiments, the pharmaceutical compositions of this disclosure are administered in the form of suppositories for rectal administration. These compositions can be prepared by mixing a compound of this disclosure with a suitable non-irritating excipient which is solid at room temperature (it) but liquid at the rectal temperature and therefore will melt in the rectum to release the active components. Such materials include, but are not limited to, cocoa butter, beeswax, and polyethylene glycols.

In some embodiments, the pharmaceutical compositions of this disclosure are administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

In some embodiments, the pharmaceutical compositions of this disclosure are administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this disclosure can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the compounds described herein and one or more additional compounds (e.g., one or more additional compounds, drugs, or agents used for the treatment of cancer or any other condition or disease, including conditions or diseases known to be associated with or caused by cancer), both the compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds of this disclosure. Alternatively, those agents can be part of a single dosage form, mixed together with the compounds of this disclosure in a single composition.

In some aspects, the pharmaceutical compositions disclosed herein can be included in a container, pack, or dispenser together with instructions for administration.

### VI. Methods of Treatment

Disclosed herein, in some embodiments, are methods of administering a composition described herein to a subject. Some embodiments relate to use a composition described herein, such as administering the composition to a subject. Some embodiments relate to a method of treating a disorder in a subject in need thereof. Some embodiments relate to use of a composition described herein in the method of treatment.

In one embodiment, provided herein is a method for the treatment of a disease or disorder associated with abnormal cell growth, such as cancer, in a subject in need thereof, comprising administering to the subject a therapeutically effective amount of a compound of any of Formulae (I)-(IV) or Formula (A), or a pharmaceutically acceptable salt thereof. In another embodiment, provided herein is a method for the treatment of cancer in a subject in need thereof, comprising administering to the subject an effective amount of a compound of any of Formulae (I)-(IV) or Formula (A), or a pharmaceutically acceptable salt thereof.

In some embodiments, the method further comprises administering a second therapeutic agent (e.g., an anticancer therapeutic agent) to the subject. In another embodiment, provided is a method for the treatment of a disease or disorder associated with abnormal cell growth, such as cancer, in a subject in need thereof, comprising administering to the subject an amount of a compound of any of Formulae (I)-(IV) or Formula (A), or a pharmaceutically acceptable salt thereof, in combination with an amount of an additional therapeutic agent (e.g., an anticancer therapeutic agent), wherein the amounts are together effective in treating said abnormal cell growth, such as cancer.

In another embodiment, provided is a compound of any of Formulae (I)-(IV) or Formula (A), or a pharmaceutically acceptable salt thereof, for use in the treatment of abnormal cell growth, such as cancer, in a subject. In a further embodiment, provided is the use of a compound of any of Formulae (I)-(IV) or Formula (A), or a pharmaceutically acceptable salt thereof, for the treatment of abnormal cell growth, such as cancer, in a subject.

In a further embodiment, provided is the use of a compound of any of Formulae (I)-(IV) or Formula (A), or a pharmaceutically acceptable salt thereof, for the manufacture of a medicament, e.g., a medicament for the treatment of abnormal cell growth, such as cancer.

In some embodiments of the compounds, compositions, methods and uses described herein, the compound of any of Formulae (I)-(IV) or Formula (A), or a pharmaceutically acceptable salt thereof, degrades GSPT1. In frequent embodiments of each of the methods and uses herein, the abnormal cell growth is cancer. In some such embodiments, the cancer is mediated by GSPT1.

In some embodiments, the method of treatment comprises administering to a subject in need thereof, a first compound comprising a GSPT1 degrader of any of Formulae (I)-(IV) or Formula (A), or a pharmaceutically acceptable salt thereof, and a second compound comprising an FLT3 pathway inhibitor, a RAS-RAF-MEK-ERK pathway inhibitor, or a PI3K-AKT-mTOR pathway inhibitor or activator. Some embodiments include administering a composition described herein to a subject with the disorder. In some embodiments, the administration treats the disorder in the subject. In some embodiments, the composition treats the disorder in the subject. In some embodiments, the treatment comprises prevention, inhibition, or reversion of the disorder in the subject. In some embodiments, the subject has cancer.

According to an aspect of the present disclosure, a method of treating a GSPT1-mediated disease disclosed herein comprises administering to a subject with GSPT1-mediated disease a compound disclosed herein (e.g. a compound of any of Formulae (I)-(IV) or Formula (A)), or a pharmaceutically acceptable salt thereof. In some embodiments, the compound is a compounds disclosed in Table 1, or a pharmaceutically acceptable salt thereof.

The methods disclosed herein contemplate administration of an effective amount of a compound or composition to achieve the desired or stated effect. Typically, the compounds or compositions of the disclosure will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound. In some embodiments, the first compound and the second compound are coadministered to the subject. In some embodiments, the first compound and the second compound are each administered separately to the subject. In some embodiments, the subject is a mammal. In some embodiments, the subject is a human.

In some aspects, provided herein is a compound or a pharmaceutically acceptable salt thereof for preventing or treating a disease or condition. In some aspects, provided herein is a heterobifunctional compound described herein for preventing or treating a disease or condition.

In some aspects, provided herein is a compound or a pharmaceutically acceptable salt thereof for treating or preventing one or more diseases or conditions disclosed herein in a subject in need thereof. In some embodiments, the disease or condition is a GSPT1-mediated disease or condition. In some embodiments, the diseases or conditions are cancer, inflammation, auto-immune disease, viral infections, and immunological diseases. In some embodiments, the GSPT1-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, and myeloma. In some embodiments, the disease or condition is a cancer. In some embodiments, the disease or condition is a GSPT1-mediated cancer.

In some embodiments, the GSPT1-mediated cancer is selected from the group consisting of mesothelioma, leukemias, and lymphomas such as cutaneous T-cell lymphomas (CTCL), noncutaneous peripheral T-cell lymphomas, lymphomas associated with human T-cell lymphotrophic vims (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma, myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), liver cancer, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST), cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

In some embodiments, the GSPT1-mediated disease is a relapsed cancer.

In some embodiments, the GSPT1-mediated disease is refractory to one or more previous treatments.

In some embodiments, the methods include the administration of a therapeutically effective amount of one or more of the compounds or compositions described herein to a subject (e.g., a mammalian subject, e.g., a human subject) who is in need of, or who has been determined to be in need of, such treatment. In some embodiments the methods disclosed include selecting a subject and administering to the subject an effective amount of one or more of the compounds or compositions described herein, and optionally repeating administration as required for the prevention or treatment of cancer.

In some embodiments, subject selection can include obtaining a sample from a subject (e.g., a candidate subject) and testing the sample for an indication that the subject is suitable for selection. In some embodiments, the subject can be confirmed or identified, e.g. by a health care professional, as having had, having an elevated risk to have, or having a condition or disease. In some embodiments, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some embodiments, exhibition of a positive immune response towards a condition or disease can be made from patient records, family history, or detecting an indication of a positive immune response. In some embodiments, multiple parties can be included in subject selection. For example, a first party can obtain a sample from a candidate subject and a second party can test the sample. In some embodiments, subjects can be selected or referred by a medical practitioner (e.g., a general practitioner). In some embodiments, subject selection can include obtaining a sample from a selected subject and storing the sample or using the in the methods disclosed herein. Samples can include, e.g., cells or populations of cells.

In some embodiments, methods of treatment can include a single administration, multiple administrations, and repeating administration of one or more compounds disclosed herein as required for the prevention or treatment of the disease or condition disclosed herein (e.g., an GSPT1-mediated disease). In some embodiments, methods of treatment can include assessing a level of disease in the subject prior to treatment, during treatment, or after treatment. In some embodiments, treatment can continue until a decrease in the level of disease in the subject is detected.

The terms “subject” or “patient” as used herein, refers to any animal subject. In some instances, the subject is a mammal. In some instances, the term “subject,” as used herein, refers to a human (e.g., a man, a woman, or a child). Subjects may include, e.g., human or veterinary patients, or human or veterinary subjects participating in clinical trials.

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compounds, compositions and methods of the present disclosure. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present disclosure. In certain embodiments, the subject has an elevated risk of developing one or more GSPT1-mediated diseases. Exemplary GSPT1-mediated diseases that can be treated with compounds include, for example, cancers of brain, stomach, gastrointestinal tracts, liver, biliary passage, breast, ovary, cervix, prostate, testis, penile, genitourinary tract, esophagus, larynx, skin, lung, pancreas, thyroid, glands, bladder, kidney, muscle, bone, and cancers of the hematopoietic system, such as myeloproliferative neoplasms, including essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, myeloma, and other hematologic malignancies.

In some embodiments, the compound is more efficacious for treating a condition such as a cancer than an existing drug. For example, the compound may be effective at a lower dose than the existing drug. In some embodiments, the compound is more efficacious than a known cereblon modulator. In some embodiments, the compound is more efficacious than CC-90009. In some embodiments, the compound is more efficacious than a known drug at reducing cell viability. For example, the compound may be more efficacious than CC-90009 at reducing cancer cell viability.

In some embodiments, the compound in combination with a second compound such as an FLT3 pathway inhibitor, a RAS-RAF-MEK-ERK pathway inhibitor, a PI3K-AKT-mTOR pathway inhibitor or activator is more efficacious for treating a condition such as cancer than an existing drug. The second compound may be an FLT3 pathway inhibitor or an FLT3 inhibitor. In some embodiments, the second compound comprises an FLT3 pathway inhibitor. In some embodiments, the second compound comprises an FLT3 inhibitor. In some embodiments, the FLT3 inhibitor comprises Gilteritinib, Midostaurin, Sorafenib, Sunitinib, Lestaurtinib, Quizartinib, Crenolanib or Sitravatinib. In some embodiments, the FLT3 inhibitor comprises Gilteritinib. In some embodiments, the compound in combination with the second compound is effective at a lower dose than the existing drug. In some embodiments, the compound in combination with the second compound is more efficacious than a known cereblon modulator. In some embodiments, the compound in combination with the second compound is more efficacious than CC-90009. In some embodiments, the compound in combination with the second compound is more efficacious than a known drug at reducing cell viability. For example, the compound in combination with the second compound may be more efficacious than CC-90009 at reducing cancer cell viability. In some embodiments, the increased efficacy of the compound in combination with the second compound, compared to another drug, is achieved.

In some embodiments, the second compound comprises a RAS-RAF-MEK-ERK pathway inhibitor. In some embodiments, the RAS-RAF-MEK-ERK pathway inhibitor comprises Vemurafenib, Dabrafenib, Encorafenib, SB590885, PLX4720, XL281, RAF265, Trametinib, Binimetinib, Cobimetinib, Selumetinib, CI-1040, or PD0325901. In some embodiments, the second compound comprises a PI3K-AKT-mTOR pathway inhibitor or activator. In some embodiments, the PI3K-AKT-mTOR pathway inhibitor or activator comprises Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, or Sarcopoterium.

In some embodiments, the second compound is a chemotherapy reagent, including, e.g., an alkylating agent (e.g., thiotepa or cyclophosphamide), an antimetabolite (e.g., 5′-azacitidine, 5-fluorouracil, gemcitabine, capecitabine, cladribine, clofarabine, cytarabine, or fludarabine), an anti-tumor antibiotic (e.g., doxorubicin or daunorubicin), a topoisomerase inhibitor (e.g, etoposide, topotecan or irinotecan), a mitotic inhibitor (e.g, paclitaxel, docetaxel, vincristine, vinorelbine or vinblastine), or a plant alkaloid (e.g., a vinca alkaloid). In some embodiments, the chemotherapy reagent is 5′-azacitidine.

In some embodiments, the compounds disclosed herein can selectively affect GSPT1-mediated disease cells compared to WT (wild type) cells (i.e., a heterobifunctional compound able to kill or inhibit the growth of an GSPT1-mediated disease cell while also having a relatively low ability to lyse or inhibit the growth of a WT cell), e.g., possess a GI₅₀ for one or more GSPT1-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the GSPT1-mediated disease cells.

In some embodiments, the compound has an IC₅₀ below that of CC-90009 in cells. In some embodiments, the cells include a cell line. In some cases, the cell line includes MV4;11 cells. In some cases, the cell line includes MOLM-13 cells. In some cases, the IC₅₀ of the compound is determined based on treating the cells with the compound alone. In some cases, the IC₅₀ of the compound is below 100 nM. In some cases, the IC₅₀ of the compound is below 100 nM. In some cases, the IC₅₀ of the compound is below 75 nM. In some cases, the IC₅₀ of the compound is below 50 nM. In some cases, the IC₅₀ of the compound is below 25 nM. In some cases, the IC₅₀ of the compound is below 10 nM. In some cases, the IC₅₀ of the compound is below 9 nM. In some cases, the IC₅₀ of the compound is below 8 nM. In some cases, the IC₅₀ of the compound is below 7 nM. In some cases, the IC₅₀ of the compound is below 6 nM. In some cases, the IC₅₀ of the compound is below 5 nM. In some cases, the IC₅₀ of the compound is below 4 nM. In some cases, the IC₅₀ of the compound is below 3 nM. In some cases, the IC₅₀ of the compound is below 2 nM. In some cases, the IC₅₀ of the compound is below 1 nM. In some cases, the IC₅₀ of the compound is below 0.5 nM. In some cases, the IC₅₀ of the compound is below 0.25 nM. In some cases, the IC₅₀ of the compound is above 100 nM. In some cases, the IC₅₀ of the compound is above 100 nM. In some cases, the IC₅₀ of the compound is above 75 nM. In some cases, the IC₅₀ of the compound is above 50 nM. In some cases, the IC₅₀ of the compound is above 25 nM. In some cases, the IC₅₀ of the compound is above 10 nM. In some cases, the IC₅₀ of the compound is above 9 nM. In some cases, the IC₅₀ of the compound is above 8 nM. In some cases, the IC₅₀ of the compound is above 7 nM. In some cases, the IC₅₀ of the compound is above 6 nM. In some cases, the IC₅₀ of the compound is above 5 nM. In some cases, the IC₅₀ of the compound is above 4 nM. In some cases, the IC₅₀ of the compound is above 3 nM. In some cases, the IC₅₀ of the compound is above 2 nM. In some cases, the IC₅₀ of the compound is above 1 nM. In some cases, the IC₅₀ of the compound is above 0.5 nM. In some cases, the IC₅₀ of the compound is above 0.5 nM. In some cases, the IC₅₀ of the compound is above 0.25 nM. Some examples of IC₅₀ values and ranges for compounds are shown in Table 2.

In some embodiments, the compound in combination with a second compound such as an FLT3 pathway inhibitor, a RAS-RAF-MEK-ERK pathway inhibitor, a PI3K-AKT-mTOR pathway inhibitor or activator, has an IC₅₀ below that of CC-90009 in cells. The second compound may be an FLT3 pathway inhibitor or an FLT3 inhibitor such as Gilteritinib. In some embodiments, the cells include a cell line. In some cases, the cell line includes MV4;11 cells. In some cases, the cell line includes MOLM-13 cells. In some cases, the IC₅₀ is determined based on treating the cells with the compound in combination with the second compound (e.g. gilteritinib). In some cases, the IC₅₀ of the compound in combination with the second compound is below 100 nM. In some cases, the IC₅₀ of the compound in combination with the second compound is below 100 nM. In some cases, the IC₅₀ of the compound in combination with the second compound is below 75 nM. In some cases, the IC₅₀ of the compound in combination with the second compound is below 50 nM. In some cases, the IC₅₀ of the compound in combination with the second compound is below 25 nM. In some cases, the IC₅₀ of the compound in combination with the second compound is below 10 nM. In some cases, the IC₅₀ of the compound in combination with the second compound is below 9 nM. In some cases, the IC₅₀ of the compound in combination with the second compound is below 8 nM. In some cases, the IC₅₀ of the compound in combination with the second compound is below 7 nM. In some cases, the IC₅₀ of the compound in combination with the second compound is below 6 nM. In some cases, the IC₅₀ of the compound in combination with the second compound is below 5 nM. In some cases, the IC₅₀ of the compound in combination with the second compound is below 4 nM. In some cases, the IC₅₀ of the compound in combination with the second compound is below 3 nM. In some cases, the IC₅₀ of the compound in combination with the second compound is below 2 nM. In some cases, the IC₅₀ of the compound in combination with the second compound is below 1 nM. In some cases, the IC₅₀ of the compound in combination with the second compound is below 0.5 nM. In some cases, the IC₅₀ of the compound in combination with the second compound is below 0.25 nM. In some cases, the IC₅₀ of the compound is above 100 nM. In some cases, the IC₅₀ of the compound is above 100 nM. In some cases, the IC₅₀ of the compound is above 75 nM. In some cases, the IC₅₀ of the compound is above 50 nM. In some cases, the IC₅₀ of the compound is above 25 nM. In some cases, the IC₅₀ of the compound is above 10 nM. In some cases, the IC₅₀ of the compound is above 9 nM. In some cases, the IC₅₀ of the compound is above 8 nM. In some cases, the IC₅₀ of the compound is above 7 nM. In some cases, the IC₅₀ of the compound is above 6 nM. In some cases, the IC₅₀ of the compound is above 5 nM. In some cases, the IC₅₀ of the compound is above 4 nM. In some cases, the IC₅₀ of the compound is above 3 nM. In some cases, the IC₅₀ of the compound is above 2 nM. In some cases, the IC₅₀ of the compound is above 1 nM. In some cases, the IC₅₀ of the compound is above 0.5 nM. In some cases, the IC₅₀ of the compound is above 0.5 nM. In some cases, the IC₅₀ of the compound is above 0.25 nM.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The method may include administering the compound to the subject by any route of administration described herein. In one embodiment, the compound is administered to the subject orally, parenterally, intradermally, subcutaneously, topically, or rectally.

In some embodiments, the method further comprises administering to the subject an additional therapeutic regimen for treating cancer, inflammatory disorders, or autoimmune diseases.

In some embodiments, the additional therapeutic regimen is selected from the group consisting of surgery, chemotherapy, radiation therapy, hormone therapy, targeted therapy, and immunotherapy.

Some embodiments include administering an additional compound. Such additional compounds may preferably be a kinase inhibitor, in particular, FLT3 pathway inhibitor (e.g. Gilteritinib, Midostaurin, Sorafenib, Sunitinib, Lestaurtinib, Quizartinib, Crenolanib or Sitravatinib), MAPK pathway inhibitor, RAS-RAF-MEK-ERK pathway inhibitor (e.g. Vemurafenib, Dabrafenib; Encorafenib, SB590885, PLX4720, XL281, RAF265, Trametinib, Binimetinib, Cobimetinib, Selumetinib, CI-1040, or PD0325901), or PI3K-AKT-mTOR pathway inhibitor or activators (e.g. Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, or Sarcopoterium).

Some embodiments include administering to a subject in need thereof a first compound. In some embodiments, the first compound is a GSPT1 degrader. In some embodiments, the first compound is a compound described herein (e.g. a compound of any of Formulae (I)-(IV) or Formula (A), or a pharmaceutically acceptable salt thereof. In some embodiments, the first compound comprises any one of the compounds in Table 1, or a pharmaceutically acceptable salt thereof.

In some embodiments, the first compound does not bind JAK. In some embodiments, the first compound includes a truncated JAK binding moiety. In some embodiments, the first compound does not include a JAK binding moiety. Some embodiments include administering to the subject a second compound comprising an FLT3 pathway inhibitor, a RAS-RAF-MEK-ERK pathway inhibitor, a PI3K-AKT-mTOR pathway inhibitor or activator. In some embodiments, the second compound includes an FLT3 pathway inhibitor. In some embodiments, the second compound includes a RAS-RAF-MEK-ERK pathway inhibitor. In some embodiments, the second compound includes a PI3K-AKT-mTOR pathway inhibitor or activator. Some embodiments include a method of treatment, comprising: administering to a subject in need thereof, a first compound comprising a GSPT1 degrader and a second compound comprising an FLT3 pathway inhibitor, a RAS-RAF-MEK-ERK pathway inhibitor, a PI3K-AKT-mTOR pathway inhibitor or activator. In some embodiments, the first compound and/or the second compound is administered to the subject as a pharmaceutical composition comprising a pharmaceutically acceptable carrier. In some embodiments, the first compound and the second compound are coadministered to the subject. In some embodiments, the first compound and the second compound are each administered separately to the subject.

Some embodiments include a method of treating or preventing cancer comprising administering to a subject in need thereof a compound that has degrader activity for GSPT1 in combination with one or more additional therapeutic agents, wherein the additional therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

In some embodiments, provided is a method of treating or preventing cancer comprising administering to a subject in need thereof a combination comprising (a) a compound of any of Formulae (I)-(IV) or Formula (A), or a pharmaceutically acceptable salt thereof; and (b) a second therapeutic agent.

In some embodiments, the second or more therapeutic agent(s) inhibits a FMS-like tyrosine kinase 3 gene (FLT3) pathway. In one embodiment, the second or more therapeutic agent(s) inhibits FLT3. Examples of FLT3 inhibitors include Gilteritinib, Midostaurin, Sorafenib, Sunitinib, Lestaurtinib, Quizartinib, Crenolanib or Sitravatinib. In some embodiments, the FLT3 inhibitor includes Gilteritinib. In some embodiments, the second compound includes an FLT3 pathway inhibitor (e.g. Gilteritinib).

In some embodiments, the second or more therapeutic agent(s) inhibits PI3K/AKT/mTOR pathway. In one embodiment, the second or more therapeutic agent(s) inhibits PI3K. In one embodiment, the second or more therapeutic agent(s) inhibits AKT. In one embodiment, the second or more therapeutic agent(s) inhibits mTOR. In some embodiments, the second compound includes a PI3K-AKT-mTOR pathway inhibitor or activator. Examples of RAS-RAF-MEK-ERK pathway inhibitors include Vemurafenib, Dabrafenib; Encorafenib, SB590885, PLX4720, XL281, RAF265, Trametinib, Binimetinib, Cobimetinib, Selumetinib, CI-1040, or PD0325901. In some embodiments, the second compound includes a RAS-RAF-MEK-ERK pathway inhibitor.

In some embodiments, the second or more therapeutic agent(s) inhibits MAPK pathway. In some embodiments, the second or more therapeutic agent(s) inhibits RAS/RAF/MEK/ERK pathway. In some embodiments, the second or more therapeutic agent(s) inhibits RAS. In some embodiments, the second or more therapeutic agent(s) inhibits RAF. In some embodiments, the second or more therapeutic agent(s) inhibits MEK. In some embodiments, the second or more therapeutic agent(s) inhibits ERK. In some embodiments, the second compound includes a RAS-RAF-MEK-ERK pathway inhibitor. Examples of PI3K-AKT-mTOR pathway inhibitors or activators include Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, Sarcopoterium. In some embodiments, the second compound includes a PI3K-AKT-mTOR pathway inhibitor. In some embodiments, the second compound includes a PI3K-AKT-mTOR pathway activator.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The terms “comprising” and “including” are used in their open, non-limiting sense.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (see-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to an alkyl group that may consist of 1 carbon atom, 2 carbon atoms, 3 carbon atoms, 4 carbon atoms, 5 carbon atoms or 6 carbon atoms, including straight-chain alkyl and branched-chain alkyl groups.

“Allyl,” as used herein, refers a —CH₂CH═CH₂ group.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term -C_{x-y}alkenylene- refers to a substituted or unsubstituted alkenylene chain with from x to y carbons in the alkenylene chain. For example, -C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term -C_{x-y}alkynylene- refers to a substituted or unsubstituted alkynylene chain with from x to y carbons in the alkenylene chain. For example, -C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene).

In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). The term -C_{x-y}alkylene- refers to a substituted or unsubstituted alkylene chain with from x to y carbons in the alkylene chain. For example -C₁₋₆alkylene- may be selected from methylene, ethylene, propylene, butylene, pentylene, and hexylene, any one of which is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₈ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

“Aryl” refers to a radical derived from an aromatic monocyclic or aromatic multicyclic hydrocarbon ring system by removing a hydrogen atom from a ring carbon atom, wherein the ring system contains at least one aromatic ring. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2)π-electron system in accordance with the Hürkel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂.

“Heteroaryl”, refers to a radical derived from a 3- to 18-membered aromatic ring radical (i.e. 3-18 membered heteroaryl) that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2)π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, a heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 10 ring atoms (4-10 membered heterocyclyl). In certain embodiments, a hetercyclyl group comprises 4 to 8 ring atoms (4-8 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to provide either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

“Carbocycle” or “carbocyclyl” refers to a saturated, unsaturated or aromatic ring system in which each ring atom of the ring system is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. In some embodiments, the carbocycle is an aryl. In some embodiments, the carbocycle is a cycloalkyl. In some embodiments, the carbocycle is a cycloalkenyl. In some embodiments, the carbocycle contains a triple bond. Unless stated otherwise specifically in the specification, a carbocycle can be optionally substituted.

“Cycloalkyl” refers to a fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

“Spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-carbocyclyl group, as defined herein.

A “halo” or “halogen” group refers to fluorine, chlorine, bromine or iodine.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

A “hydroxy” group refers to an —OH group.

A “nitro” group refers to a —NO₂ group.

An “oxo” group refers to the ═O substituent.

The term “length” when refers to a moiety means the smallest number of carbon and/or hetero atoms from one end to the other end of the moiety. When it refers to the linker, it means the smallest number of atoms from the end connects to the TRK ligand and the end connects to the Degradation Tag. It applies to both situations where the linker is linear or branched, and where the linker comprises a ring system.

The term “substituted,” means that the specified group or moiety bears one or more substituents independently selected from C₁ [symbol] ₄ alkyl, aryl, heteroaryl, aryl [symbol] ₁ [symbol] ₄ alkyl-,-heteroaryl [symbol] ₁ [symbol] ₄ alkyl C₁ [symbol] ₄ haloalkyl, [symbol] C₁-C₄ alkyl, [symbol] C₁-C₄ alkylphenyl, -C₁ [symbol] ₄ alkyl [symbol] H, [symbol] C₁ [symbol] ₄ haloalkyl, halo, [symbol] H, [symbol] H₂, [symbol] ₁ [symbol] ₄ alkyl [symbol] H₂, [symbol] (C₁ [symbol] ₄ alkyl)(C₁ [symbol] ₄ alkyl), [symbol] H(C₁ [symbol] ₄ alkyl), [symbol] (C₁ [symbol] ₄ alkyl)(C₁ [symbol] ₄ alkylphenyl), [symbol] H(C₁ [symbol] ₄ alkylphenyl), cyano, nitro, oxo, [symbol] O₂H, [symbol] (O)OC₁ [symbol] ₄ alkyl, [symbol] ON(C₁ [symbol] ₄ alkyl)(C₁ [symbol] ₄ alkyl), [symbol] ONH(C₁ [symbol] ₄ alkyl), [symbol] ONH₂, [symbol] HC(O)(C₁ [symbol] ₄ alkyl), [symbol] HC(O)(phenyl), [symbol] (C₁ [symbol] ₄ alkyl)C(O)(C₁ [symbol] ₄ alkyl), [symbol] (C₁ [symbol] ₄ alkyl)C(O)(phenyl), [symbol] (O)C₁ [symbol] ₄ alkyl, [symbol] (O)C₁ [symbol] ₄ alkylphenyl, [symbol] (O)C₁ [symbol] ₄ haloalkyl, [symbol] C(O)C₁ [symbol] ₄ alkyl, —SO₂(C₁ [symbol] ₄ alkyl), —SO₂(phenyl), —SO₂(C₁ [symbol] ₄ haloalkyl), —SO₂NH₂, [symbol] O₂NH(C₁ [symbol] ₄ alkyl), [symbol] O₂NH(phenyl), —NHSO₂(C₁ [symbol] ₄ alkyl), —NHSO₂(phenyl), and [symbol] HSO₂(C₁ [symbol] ₄ haloalkyl).

The term “null” or “absent” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present disclosure when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present disclosure is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NRⁿR^{o}, COR^{m}, CO₂R^{m}, CONRⁿR^{o}, SOR^{m}, SO₂R^{m}, SO₂NRⁿR^{o}, NRⁿCOR^{o}, NR^{m}C(O)NRⁿR^{o}, NRⁿSOR^{o}, NRⁿSO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈ alkyl, C₃-C₇ carbocyclyl, 3-7 membered heterocyclyl, C₂-C₈ alkenyl, C₂-C₈ alkynyl, aryl, and heteroaryl, wherein R^{m}, Rⁿ, and R^{o} are independently selected from null, hydrogen, C₁-C₈ alkyl, C₂-C₈ alkenyl, C₂-C₈ alkynyl, C₃-C₇ carbocyclyl, 3-7 membered heterocyclyl, aryl, and heteroaryl, or Rⁿ and R^{o} together with the atom to which they are connected form a 3-8 membered carbocyclyl or heterocyclyl ring.

The term “combination therapy” or “combination” or “in combination with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e. the effect achieved when the active ingredients used together is greater than the sum of the effects that results from using the compounds separately. A synergistic effect can be attained when the active ingredients are: (1) co formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

A “therapeutic agent” as used herein refers to a therapy, e.g., a molecule, including but not limited to, a chemical compound, peptide, antibody, antibody fragment, antibody conjugate, or nucleic acid; a gene or cell therapy; or a radiation therapy, which is therapeutically active or enhances the therapeutic activity when administered to a subject in combination with a compound of the present disclosure or which reduces one or more side effects of the compound of the present disclosure when administered to a subject in combination with a compound of the present disclosure.

“Cancer” means any cancer caused by the uncontrolled proliferation of aberrant cells, such as tumors, neoplasms, carcinomas, sarcomas, leukemias, lymphomas, and the like. Cancer cells can spread locally or through the bloodstream and lymphatic system to other parts of the body. For example, cancers include, but are not limited to, mesothelioma, leukemias, and lymphomas such as cutaneous T-cell lymphomas (CTCL), noncutaneous peripheral T-cell lymphomas, lymphomas associated with human T-cell lymphotrophic vims (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), liver cancer, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). Additional exemplary forms of cancer which may be treated by the compounds and compositions described herein include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

The second agent can be an anti-cancer agent. The term “anti-cancer” or “anti-cancer agent” pertains to an agent which treats a cancer (i.e., a compound, antibody, etc. which is useful in the treatment of a cancer). The anti-cancer effect may arise through one or more mechanisms, including, but not limited to, the regulation of cell growth or proliferation, the inhibition of angiogenesis (the formation of new blood vessels), the inhibition of metastasis (the spread of a tumor from its origin), the inhibition of invasion (the spread of tumor cells into neighboring normal structures), the inhibition of a checkpoint molecule, or the promotion of apoptosis.

The anti-cancer agent is can be an anti-proliferative agent or an immunomodulatory agent. In one embodiment, the second agent is an immunomodulatory agent.

The term “antiproliferative” or “antiproliferative agent” as used herein pertains to an agent, which inhibits cell growth or cell proliferation. The anti-proliferative agent can be a cytotoxic agent (e.g., alkylating agent, antimetabolites, etc.), a targeted agent (e.g., EGF inhibitor, Tyrosine protein kinase inhibitor, angiogenesis inhibitor, etc.), or a hormonal agent (e.g., estrogens selective estrogen receptor modulators, etc.). Examples of antiproliferative agents include alkylating agents, anti-metabolites, an antibiotic, a detoxifying agent, an EGFR inhibitor, a HER2 inhibitor, a histone deacetylase inhibitor, a hormone, a mitotic inhibitor, an MTOR inhibitor, a multi-kinase inhibitor, a serine/threonine inhibitor, a tyrosine kinase inhibitor, a VEGF/VEGFR inhibitor; a taxane or taxane derivative, an aromatase inhibitor, an anthracycline, a microtubule targeting drug, a topoisomerase poison drug, an inhibitor of a molecular target or enzyme.

The term “immunomodulatory agent” is agent that modifies the immune response or the functioning of the immune system (as by the stimulation of antibody formation or the inhibition of white blood cell activity). The immunomodulatory agents can be an immunomodulator, a cytokine, a vaccine, or an anti-body.

The term “immunomodulator” is an inhibitor of an immune checkpoint molecule.

Additional cancers that the compounds and compositions described herein may be useful in preventing, treating, and studying are, for example, colon carcinoma, familiary adenomatous polyposis carcinoma, and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing's sarcoma, and plasmacytoma.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

General Chemistry Methods: All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quaternary Solvent Manager) with degasser, an autosampler (FTN), a column oven (40 [symbol] , unless otherwise indicated), a photo-diode array PDA detector. Chromatography was performed on an AcQuity UPLC BEH C18 (1.7 μm, 2.1×50 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance 111400 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 μm, 30×75 mm) at rt. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. All compounds showed >95% purity using the LCMS methods.

### Example 1: 5-((Trans-3-(3-cyclopropyl-4-(quinoxalin-2-yl)-1H-pyrazol-1-yl)cyclobutyl)methoxy)-2-(2,6-dioxopiperidin-3-yl)isoindoline-1,3-dione (GS-640)

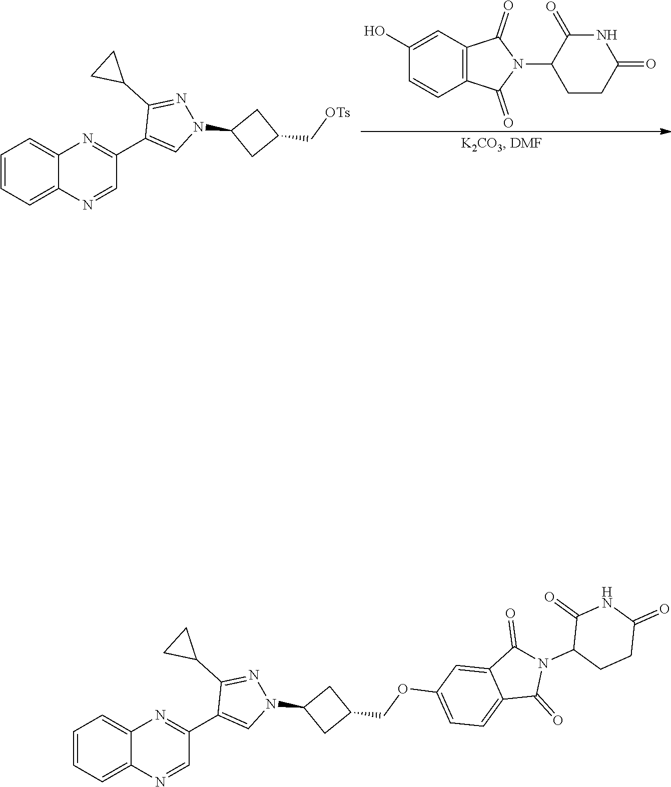 [SMILES: Cc1ccc(S(=O)(=O)OC[C@H]2C[C@H](n3cc(-c4cnc5ccccc5n4)c(C4CC4)n3)C2)cc1 ; O=C1CCC(N2C(=O)c3ccc(O)cc3C2=O)C(=O)N1 ; O=C1CCC(N2C(=O)c3ccc(OC[C@H]4C[C@H](n5cc(-c6cnc7ccccc7n6)c(C6CC6)n5)C4)cc3C2=O)C(=O)N1]

To a solution of (trans-3-(3-cyclopropyl-4-(quinoxalin-2-yl)-1H-pyrazol-1-yl)cyclobutyl)methyl 4-methylbenzenesulfonate (70 mg, 147.5 μmol) and 2-(2,6-dioxopiperidin-3-yl)-5-hydroxyisoindoline-1,3-dione (40.4 mg, 147.5 μmol) in DMF (3 mL) was added K₂CO₃ (40.8 mg, 295 μmol). After the reaction mixture was stirred at 60 [symbol] for 12 h, it was purified by reverse-phase chromatography to provide the desired product (2.1 mg, 3% yield) as a yellow solid. MS (ESI) m/z=577.3 [M+H]⁺.

### Example 2. 5-(((Trans-3-(4-(3-cyclopropoxypyridin-2-yl)-3-cyclopropyl-1H-pyrazol-1-yl)cyclobutyl)methyl)amino)-2-(2,6-dioxopiperidin-3-yl)isoindoline-1,3-dione (GS-641)

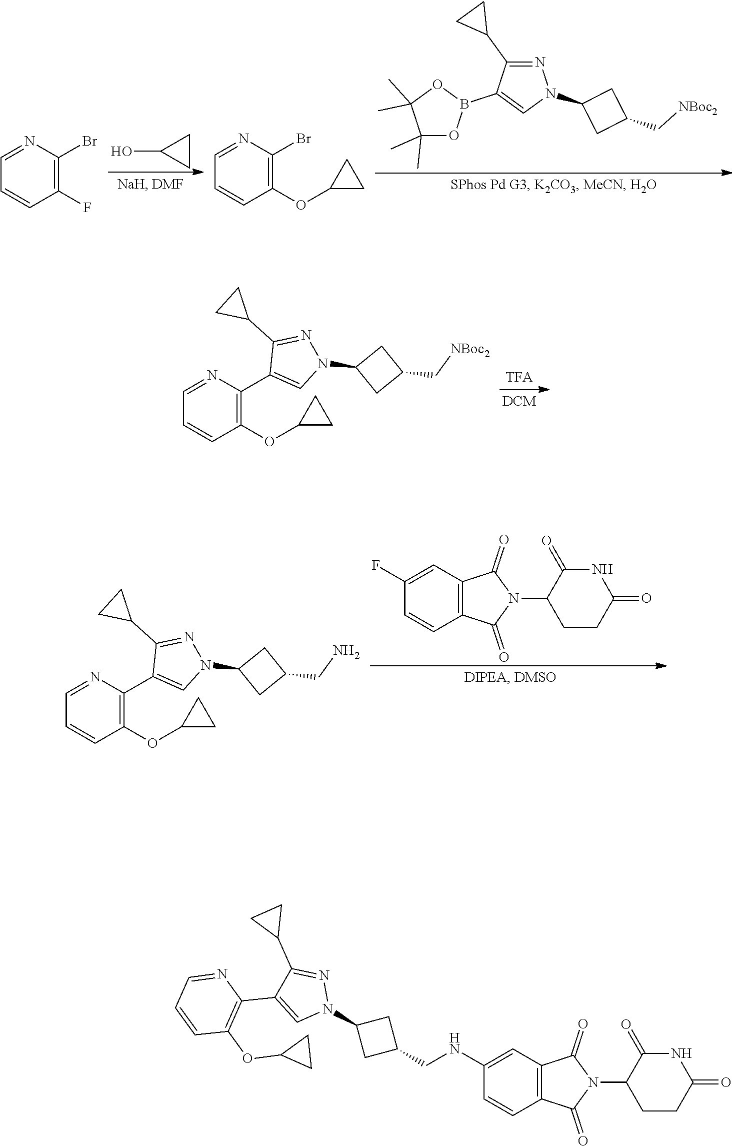

### Step 1. Synthesis of 2-bromo-3-cyclopropoxypyridine

To a solution of cyclopropanol (1.98 g, 34.09 mmol) in DMF (20 mL) was added NaH (2.73 g, 68.19 mmol) slowly at 0° C. After the reaction was stirred at 0° C. for 1 h, 2-bromo-3-fluoro-pyridine (3 [symbol] g, 17.05 mmol) was added dropwise. The resulting mixture was stirred at 70 [symbol] for 16 h. After the reaction mixture was cooled down to rt, it was poured into saturated ammonium chloride aqueous solution (200 mL) and extracted with ethyl acetate (3×60 mL). The combined organic layers were washed with saturated brine (60 mL), dried over anhydrous sodium sulfate, filtered and evaporated under reduced pressure. The resulting residue was purified by silica gel flash chromatography to provide the desired product (600 mg, 17% yield) as a light-yellow oil. MS (ESI) m/z=214.0 [M+H]⁺.

### Step 2. Synthesis of tert-butyl N-tert-butoxycarbonyl-N-((trans-3-(4-(3-(cyclopropoxy)-2-pyridyl)-3-cyclopropyl-pyrazol-1-yl)cyclobutyl)methyl)carbamate

To a solution of 2-bromo-3-(cyclopropoxy)pyridine (100.0 mg, 0.467 mmol) in MeCN (6.0 mL) and water (4.0 mL) were added tert-butyl N-tert-butoxycarbonyl-N-((trans-3-(3-cyclopropyl-4-(4,4,5,5-tetramethyl-1,3,2-dioxaborolan-2-yl)pyrazol-1-yl)cyclobutyl)methyl)carbamate (290.1 mg, 0.56 mmol), Sphos Pd G3 (45.1 mg, 0.0467 mmol) and K₂CO₃ (128.9 mg, 0.934 mmol) at rt. The reaction mixture was stirred at 100 [symbol] for 16 h under inert atmosphere. After cooling down to rt, the mixture was poured into water (20 mL) and extracted with ethyl acetate (3×20 mL). The combined organic layers were washed with brine (20 mL), dried over anhydrous sodium sulfate, filtered and evaporated under reduced pressure. The resulting residue was purified by reverse-phase chromatography to provide the desired product (171.0 mg, 70% yield) as a pale-white solid. MS (ESI) m/z=525.2 [M+H]⁺.

### Step 3. Synthesis of (trans-3-(4-(3-cyclopropoxypyridin-2-yl)-3-cyclopropyl-1H-pyrazol-1-yl)cyclobutyl)methanamine

To a solution of tert-butyl N-tert-butoxycarbonyl-N-((trans-3-(4-(3-(cyclopropoxy)-2-pyridyl)-3-cyclopropyl-pyrazol-1-yl)cyclobutyl)methyl)carbamate (220.0 mg, 0.418 mmol) in DCM (6 mL) was added TFA (3 mL) at rt. After stirring at rt for 1 h, the reaction mixture was concentrated and purified by reverse-phase chromatography to provide the desired product (130.0 mg, 95% yield) as a light-yellow oil. MS (ESI) m/z=325.0 [M+H]⁺.

### Step 4. Synthesis of 5-(((trans-3-(4-(3-cyclopropoxypyridin-2-yl)-3-cyclopropyl-1H-pyrazol-1-yl)cyclobutyl)methyl)amino)-2-(2,6-dioxopiperidin-3-yl)isoindoline-1,3-dione

To a solution of (trans-3-(4-(3-cyclopropoxypyridin-2-yl)-3-cyclopropyl-1H-pyrazol-1-yl)cyclobutyl)methanamine (180.0 mg, 0.555 mmol) and 2-(2,6-dioxo-3-piperidyl)-5-fluoro-isoindoline-1,3-dione (183.9 mg, 0.666 mmol) in DMSO (3 mL) was added DIPEA (0.72 g, 5.55 mmol) at rt. The reaction mixture was stirred at 130 [symbol] for 2 h under microwave irradiation. After cooling down to rt, the mixture was poured into water (15 mL) and extracted with ethyl acetate (3×10 mL). The combined organic layers were washed with saturated brine (15 mL), dried over anhydrous sodium sulfate, filtered and evaporated under reduced pressure. The resulting residue was purified by reverse-phase chromatography to provide the desired product (45.0 mg, 14% yield) as a yellow solid. MS (ESI) m/z=581.7 [M+H]⁺.

### Example 3. 5-((3-(3-Cyclopropyl-4-(quinoxalin-2-yl)-1H-pyrazol-1-yl)propyl)amino)-2-(2,6-dioxopiperidin-3-yl)isoindoline-1,3-dione (GS-642)

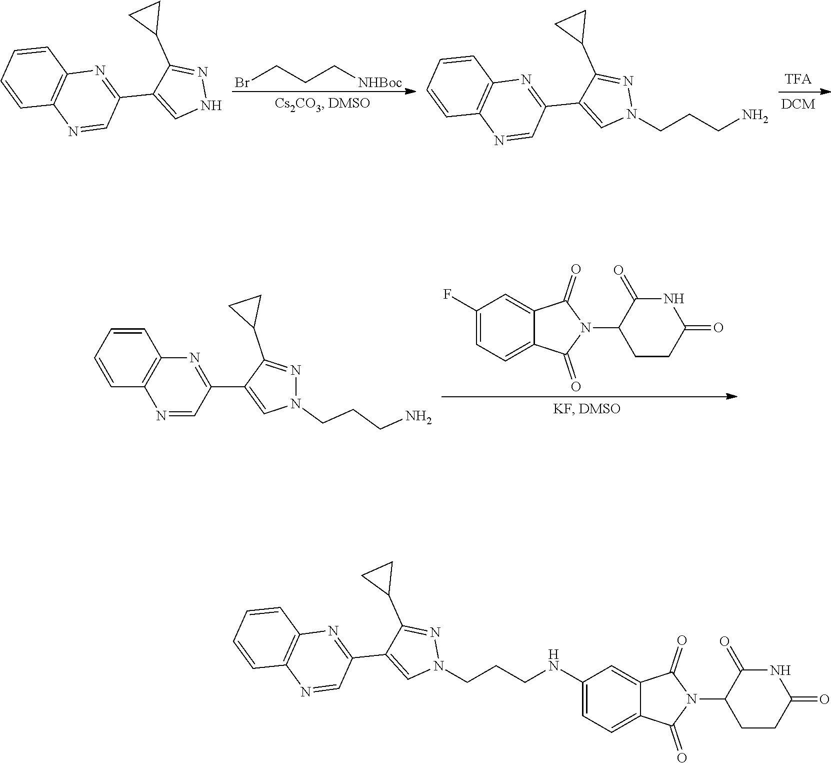

### Step 1. Synthesis of tert-butyl (3-(3-cyclopropyl-4-(quinoxalin-2-yl)-1H-pyrazol-1-yl)propyl)carbamate

To a solution of 2-(1-(piperidin-4-yl)-1H-pyrazol-4-yl)quinoxaline (40 mg, 0.17 mmol) and tert-butyl (3-bromopropyl)carbamate (85 mg, 0.34 mmol) in DMSO (3 mL) was added Cs₂CO₃ (166 mg, 0.51 mmol) at rt. Then the reaction mixture was stirred at 80° C. for 3 h. The mixture was purified by reverse-phase chromatography to provide the desired product (40 mg, 60% yield) as a light yellow solid. MS (ESI) m/z=394.5 [M+H]⁺.

### Step 2. Synthesis of 3-(3-cyclopropyl-4-(quinoxalin-2-yl)-1H-pyrazol-1-yl)propan-1-amine

A mixture of tert-butyl (3-(3-cyclopropyl-4-(quinoxalin-2-yl)-1H-pyrazol-1-yl)propyl)carbamate (40 mg, 0.10 mmol) in TFA/DCM (1:1, 4 mL) was stirred at rt for 1 h. The resulting mixture was concentrated to provide the crude product (30 mg, 100% yield) as a light yellow solid. This compound was used directly in the next step without further purification. MS (ESI) m/z=294.5 [M+H]⁺.

### Step 3. Synthesis of 5-((3-(3-cyclopropyl-4-(quinoxalin-2-yl)-1H-pyrazol-1-yl)propyl)amino)-2-(2,6-dioxopiperidin-3-yl)isoindoline-1,3-dione

To a solution of 3-(3-cyclopropyl-4-(quinoxalin-2-yl)-1H-pyrazol-1-yl)propan-1-amine (30 mg, 0.10 mmol) and 2-(2,6-dioxopiperidin-3-yl)-5-fluoroisoindoline-1,3-dione (28 mg, 0.10 mmol) in DMSO (2 mL) was added KF (30 mg, 0.50 mmol) at rt. Then the reaction mixture was stirred at 130° C. for 1 h under microwave irradiation. After cooling down to rt, the mixture was purified by reverse-phase chromatography to provide the desired product (15 mg, 27% yield) as a light yellow solid. MS (ESI) m/z=550.6 [M+H]⁺.

### Example 4. 5-(((Trans-2-(3-cyclopropyl-4-(5-fluoro-6-methylpyridin-2-yl)-1H-pyrazol-1-yl)cyclopropyl)methyl)amino)-2-(2,6-dioxopiperidin-3-yl)isoindoline-1,3-dione (GS-643)

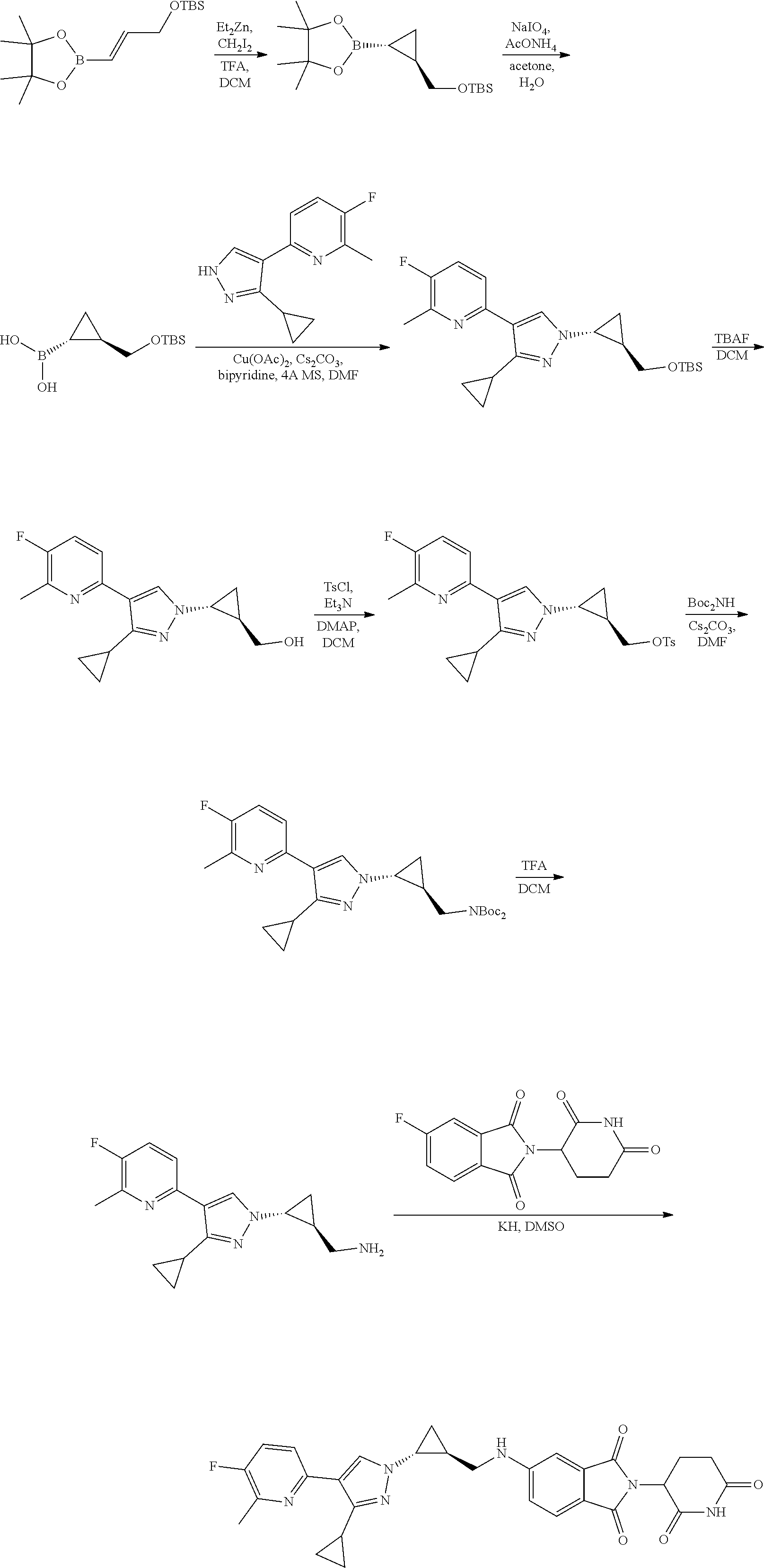

### Step 1. Synthesis of tert-butyldimethyl((trans-2-(4,4,5,5-tetramethyl-1,3,2-dioxaborolan-2-yl)cyclopropyl)methoxy)silane

To a solution of (E)-tert-butyldimethyl((3-(4,4,5,5-tetramethyl-1,3,2-dioxaborolan-2-yl)allyl)oxy)silane (1.2 g, 4.02 mmol) in DCM (50 mL) was added Et₂Zn (2.48 g, 20.13 mmol) at 0° C. After the reaction was stirred at 0° C. for 30 min, CH₂I₂ (5.4 g, 20.13 mmol) and TFA (2.3 g, 20.13 mmol) were added to the above solution at 0° C. The reaction mixture was stirred at 0° C. for 1 h, and at rt for another 2 h. The reaction was quenched with aq. NH₄Cl and extracted with DCM (2×100 mL). The combined organic layers were washed with saturated brine (80 mL), dried over anhydrous sodium sulfate and evaporated under reduced pressure. The residue was purified by silica gel chromatography to provide the desired product (800 mg, 64% yield) as colorless oil.

### Step 2. Synthesis of (trans-2-(((tert-butyldimethylsilyl)oxy)methyl)cyclopropyl)boronic acid

To a solution of tert-butyldimethyl((trans-2-(4,4,5,5-tetramethyl-1,3,2-dioxaborolan-2-yl)cyclopropyl)methoxy)silane (800 mg, 2.56 mmol) in acetone (40 mL) and water (20 mL) were added NaIO₄ (3.29 g, 15.36 mmol) and AcONH₄ (1.18 mg, 15.36 mmol) at rt. After stirring at rt for 10 h, the reaction mixture was diluted with water (100 mL) and extracted with dichloromethane (2×100 mL). The combined organic layers were washed with saturated brine (50 mL), dried over anhydrous sodium sulfate and evaporated under reduced pressure. The residue was purified by silica gel chromatography to provide the desired product (480 mg, 81% yield) as a yellow oil.

### Step 3. Synthesis of 6-(1-(trans-2-(((tert-butyldimethylsilyl)oxy)methyl)cyclopropyl)-3-cyclopropyl-1H-pyrazol-4-yl)-3-fluoro-2-methylpyridine

To a solution of 6-(3-cyclopropyl-1H-pyrazol-4-yl)-3-fluoro-2-methylpyridine (120 mg, 0.55 mmol) and (trans-2-(((tert-butyldimethylsilyl)oxy)methyl)cyclopropyl)boronic acid (250 mg, 1.10 mmol) in DMF (10 mL) were added Cu(OAc)₂ (200 mg, 1.10 mmol), Cs₂CO₃ (538 mg, 1.65 mmol), bipyridine (172 mg, 1.10 mmol) and 4 Å MS (100 mg). The reaction mixture was stirred at 100° C. for 10 h under argon. After cooling down to rt, the reaction was quenched with water (50 mL) and extracted with ethyl acetate (3×50 mL). The combined organic layers were washed with saturated brine (50 mL), dried over anhydrous sodium sulfate and evaporated under reduced pressure. The residue was purified by silica gel chromatography to provide the desired product (45 mg, 20% yield) as a yellow solid. MS (ESI) m/z=402.5 [M+H]⁺.

### Step 4. Synthesis of (trans-2-(3-cyclopropyl-4-(5-fluoro-6-methylpyridin-2-yl)-1H-pyrazol-1-yl)cyclopropyl)methanol

To a solution of 6-(1-(trans-2-(((tert-butyldimethylsilyl)oxy)methyl)cyclopropyl)-3-cyclopropyl-1H-pyrazol-4-yl)-3-fluoro-2-methylpyridine (45 mg, 0.11 mmol) in DCM (10 mL) was added TBAF (1M in THF, 1.0 mL) at rt. After stirring at rt for 6 h, the reaction mixture was concentrated and purified by reverse-phase chromatography to provide the desired product (30 mg, 93% yield) as a light-yellow solid. MS (ESI) m/z=288.2 [M+H]⁺.

### Step 5. Synthesis of (trans-2-(3-cyclopropyl-4-(5-fluoro-6-methylpyridin-2-yl)-1H-pyrazol-1-yl)cyclopropyl)methyl 4-methylbenzenesulfonate

To a solution of (trans-2-(3-cyclopropyl-4-(5-fluoro-6-methylpyridin-2-yl)-1H-pyrazol-1-yl)cyclopropyl)methanol (30 mg, 0.10 mmol) in DCM (5 mL) were added TsCl (38 mg, 0.20 mmol), Et₃N (50 mg, 0.50 mmol) and DMAP (18 mg, 0.15 mmol) at 0 [symbol] . The reaction mixture was stirred at rt for 1 h, before it was poured into water (50 mL) and extracted with DCM (3×20 mL). The combined organic layers were washed with saturated brine, dried over anhydrous sodium sulfate, filtered and evaporated under reduced pressure. The resulting residue was purified by silica gel flash chromatography to give the desired product (35 mg, 76% yield) as a white solid. MS (ESI) m/z=442.3 [M+H]⁺.

### Step 6. Synthesis of tert-butyl N-tert-butoxycarbonyl-N-((trans-2-(3-cyclopropyl-4-(5-fluoro-6-methyl-2-pyridyl)pyrazol-1-yl)cyclopropyl)methyl)carbamate

To a solution of (trans-2-(3-cyclopropyl-4-(5-fluoro-6-methylpyridin-2-yl)-1H-pyrazol-1-yl)cyclopropyl)methyl 4-methylbenzenesulfonate (35 mg, 0.08 mmol) and tert-butyl N-tert-butoxycarbonylcarbamate (34 mg, 0.16 mmol) in DMF (4 mL) was added Cs₂CO₃ (78 mg, 0.24 mmol) at rt. The reaction was stirred at 90 [symbol] for 1 h, before it was quenched with H₂O (20 mL) and extracted with EtOAc (20 mL×3). The combined organic phase was washed with brine and dried over anhydrous Na₂SO₄, filtered, and concentrated under reduced pressure. The resulting residue was purified by silica gel chromatography to provide the desired product (35 mg, 90% yield) as a yellow oil. MS (ESI) m/z=487.3 [M+H]⁺.

The remaining steps were performed according to the procedures for steps 2 to 3 of GS-642 to provide the desired product (2 mg, 11% yield over 2 steps) as a yellow solid. MS (ESI) m/z=543.3 [M+H]⁺.

### Example 5. 5-(((Trans-3-(3-cyclopropyl-4-(7-(3-hydroxyazetidin-1-yl)quinoxalin-2-yl)-1H-pyrazol-1-yl)cyclobutyl)methyl)amino)-2-(2,6-dioxopiperidin-3-yl)isoindoline-1,3-dione (GS-644)

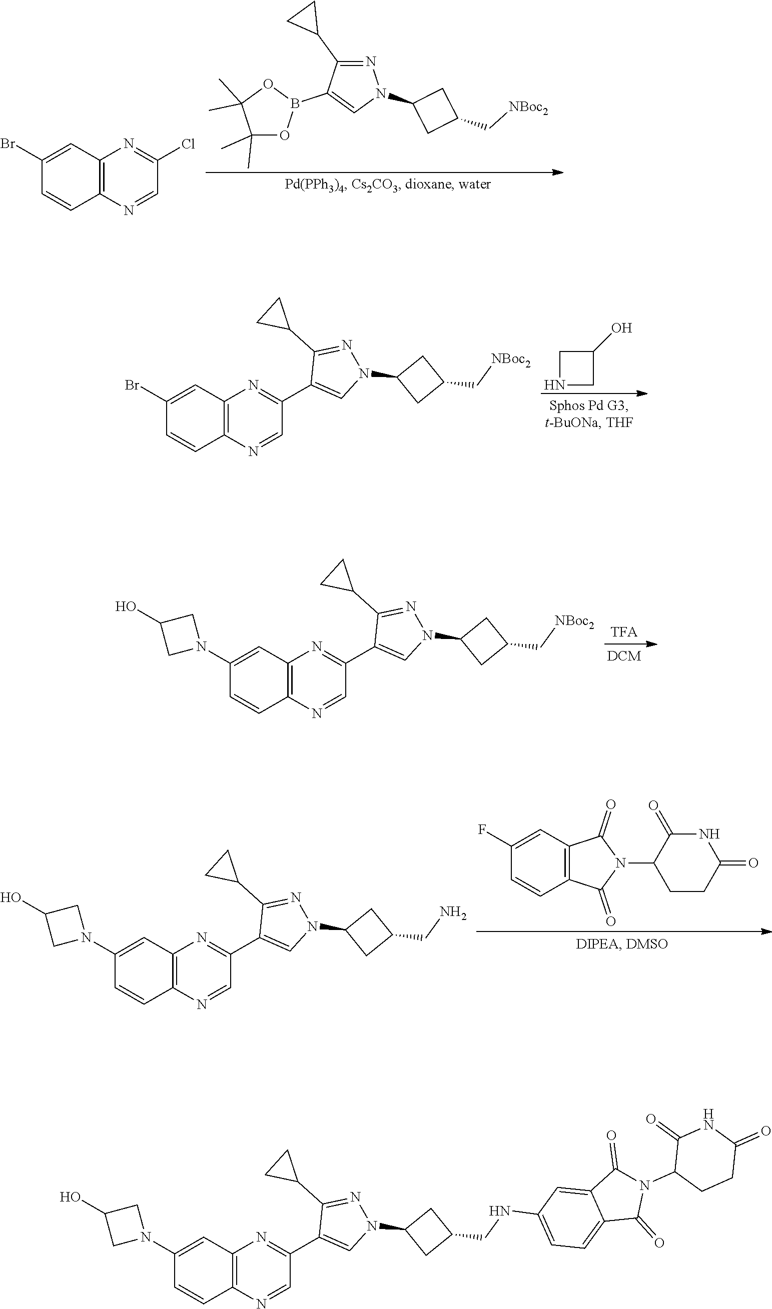

Step 1. Synthesis of tert-butyl N-((trans-3-(4-(7-bromoquinoxalin-2-yl)-3-cyclopropyl-pyrazol-1-yl)cyclobutyl)methyl)-N-tert-butoxycarbonyl-carbamate

To a solution of 7-bromo-2-chloro-quinoxaline (650 mg, 2.67 mmol) in dioxane (10 mL) and water (2 mL) were added tert-butyl N-tert-butoxycarbonyl-N-((trans-3-(3-cyclopropyl-4-(4,4,5,5-tetramethyl-1,3,2-dioxaborolan-2-yl)pyrazol-1-yl)cyclobutyl)methyl)carbamate (1.66 g, 3.20 mmol), Pd(PPh₃)₄ (308.3 mg, 0.267 mmol) and Cs₂CO₃ (1.74 g, 5.34 mmol) at rt. The reaction mixture was stirred at 100 [symbol] for 4 h under inert atmosphere. After cooling down to rt, the mixture was poured into water (50 mL) and extracted with ethyl acetate (3×30 mL). The combined organic layers were washed with saturated brine (30 mL), dried over anhydrous sodium sulfate, filtered and evaporated under reduced pressure. The resulting residue was purified by reverse-phase chromatography to provide the desired product (400 mg, 15% yield) as a pale-white solid. MS (ESI) m/z=598.3 [M+H]⁺.

### Step 2. Synthesis of tert-butyl N-tert-butoxycarbonyl-N-((trans-3-(3-cyclopropyl-4-(7-(3-hydroxyazetidin-1-yl)quinoxalin-2-yl)pyrazol-1-yl)cyclobutyl)methyl)carbamate

To a solution of tert-butyl N-((trans-3-(4-(7-bromoquinoxalin-2-yl)-3-cyclopropyl-pyrazol-1-yl)cyclobutyl)methyl)-N-tert-butoxycarbonyl-carbamate (110.0 mg, 0.184 mmol) in THF (10 mL) were added azetidin-3-ol (53.7 mg, 0.735 mmol), Sphos Pd G3 (15.9 mg, 0.018 mmol) and t-BuONa (53.0 mg, 0.551 mmol) at rt. Then the reaction mixture was stirred at 100 [symbol] for 2 h under inert atmosphere. After cooling down to rt, the mixture was poured into water (30 mL) and extracted with ethyl acetate (3×20 mL). The combined organic layers were washed with saturated brine (30 mL), dried over anhydrous sodium sulfate, filtered and evaporated under reduced pressure. The resulting residue was purified by reverse-phase chromatography to provide the desired product (50 mg, 46% yield) as a pale-white solid. MS (ESI) m/z=591.7 [M+H]⁺.

The remaining steps were performed according to the procedures for steps 3 to 4 of GS-641 to provide the desired product (0.88 mg, 1% yield over 2 steps) as a light-yellow solid. MS (ESI) m/z=647.7 [M+H]⁺.

### Example 6. 5-(((2-((3-Cyclopropyl-4-(5-fluoro-6-methylpyridin-2-yl)-1H-pyrazol-1-yl)methyl)cyclopropyl)methyl)amino)-2-(2,6-dioxopiperidin-3-yl)isoindoline-1,3-dione (GS-645)

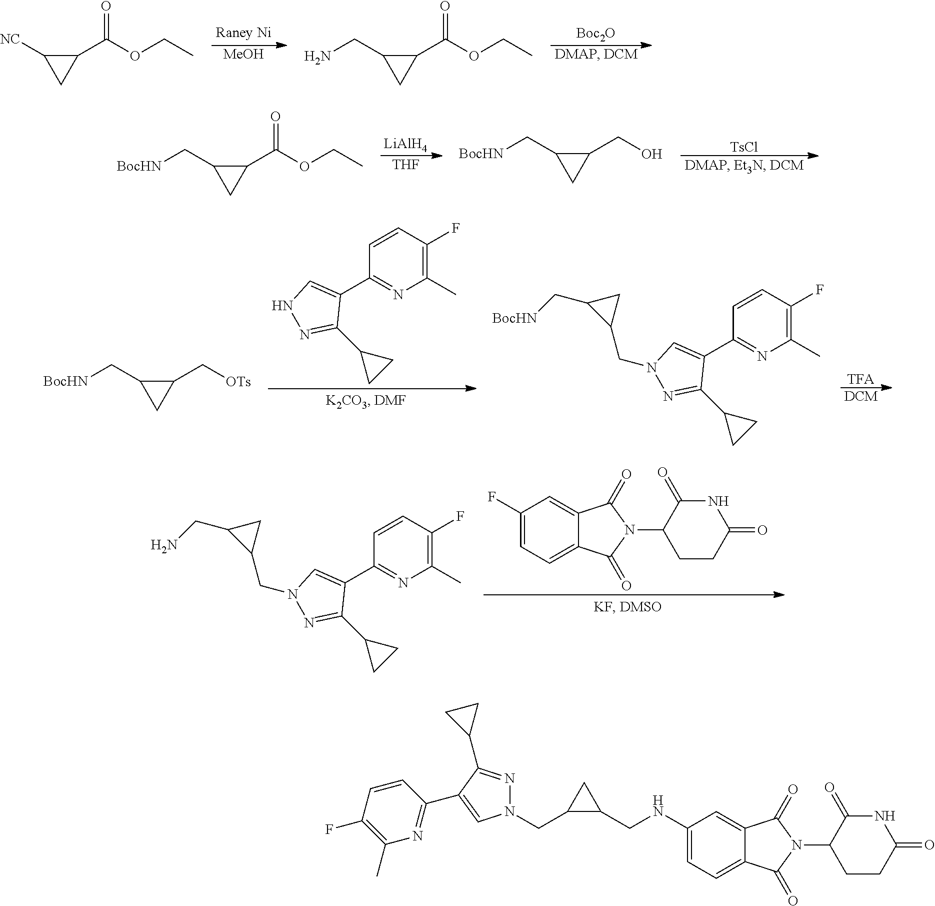

### 1. Synthesis of ethyl 2-(aminomethyl)cyclopropane-1-carboxylate

To a solution of ethyl 2-cyanocyclopropane-1-carboxylate (400 mg, 2.88 mmol) in MeOH (10 mL) was added Raney Ni (100 mg) at rt. The reaction mixture was stirred at rt for 4 h under hydrogen atmosphere. The mixture was filtered through Celite. The filtrate was concentrated under reduced pressure to provide the desired product (300 mg, 73% yield) as a colorless oil.

### Step 2. Synthesis of ethyl 2-(((tert-butoxycarbonyl)amino)methyl)cyclopropane-1-carboxylate

To a solution of ethyl 2-(aminomethyl)cyclopropane-1-carboxylate (300 mg, 2.10 mmol) in dichloromethane (20 mL) were added Boc₂O (900 mg, 4.20 mmol) and DMAP (384 mg, 3.15 mmol). After the reaction mixture was stirred at rt for 6 h, it was quenched with water (20 mL) and extracted with dichloromethane (3×20 mL). Then combined organic layers were washed with saturated brine (20 mL), dried over anhydrous sodium sulfate, filtered and evaporated under reduced pressure. The residue was purified by silica gel chromatography to provide the desired product (300 mg, 59% yield) as a colorless oil.

### Step 3. Synthesis of tert-butyl ((2-(hydroxymethyl)cyclopropyl)methyl)carbamate

To a solution of ethyl 2-(((tert-butoxycarbonyl)amino)methyl)cyclopropane-1-carboxylate (300 mg, 1.23 mmol) in THF (10 mL) was added LiAlH₄ (140 mg, 3.70 mmol) at 0 [symbol] . The reaction mixture was stirred at rt for 2 h under argon, before it was quenched with solid Na₂SO₄ and diluted with EtOAc (25 mL). The suspension was filtered through Celite and the filtrate was concentrated under reduced pressure to provide the crude product (200 mg, 81% yield). This compound was used in the next step directly without further purification.

### Step 4. Synthesis of (2-(((tert-butoxycarbonyl)amino)methyl)cyclopropyl)methyl 4-methylbenzenesulfonate

To a solution of tert-butyl ((2-(hydroxymethyl)cyclopropyl)methyl)carbamate (200 mg, 0.99 mmol) and 4-methylbenzenesulfonyl chloride (474 mg, 2.48 mmol) in dichloromethane (20 mL) were added Et₃N (500 mg, 4.97 mmol) and DMAP (121 mg, 0.99 mmol). The reaction mixture was stirred at rt for 10 h, before it was quenched with water (20 mL) and extracted with dichloromethane (2×20 mL). The combined organic layers were washed with saturated brine (30 mL), dried over anhydrous sodium sulfate, filtered and evaporated under reduced pressure. The resulting residue was purified by silica gel chromatography to provide the desired product (120 mg, 34% yield) as a white solid.

### Step 5. Synthesis of tert-butyl ((2-((3-cyclopropyl-4-(5-fluoro-6-methylpyridin-2-yl)-1H-pyrazol-1-yl)methyl)cyclopropyl)methyl)carbamate

To a solution of (2-(((tert-butoxycarbonyl)amino)methyl)cyclopropyl)methyl 4-methylbenzenesulfonate (100 mg, 0.28 mmol) and 6-(3-cyclopropyl-1H-pyrazol-4-yl)-3-fluoro-2-methylpyridine (20 mg, 0.09 mmol) in DMF (10 mL) was added K₂CO₃ (65 mg, 0.47 mmol). The reaction mixture was stirred at 80 [symbol] for 3 h. After cooling down to rt, the reaction was poured into water (50 mL) and extracted with ethyl acetate (3×50 mL). The combined organic layers were washed with saturated brine (50 mL), dried over anhydrous sodium sulfate, filtered and evaporated under reduced pressure. The resulting residue was purified by silica gel chromatography to provide the desired product (25 mg, 66% yield) as a brown solid. MS (ESI) m/z=401.8 [M+H]⁺.

The remaining steps were performed according to the procedures for steps 2 to 3 of GS-642 to provide the desired product (7 mg, 25% yield over 2 steps) as a yellow solid. MS (ESI) m/z=557.6 [M+H]⁺.

### Example 7. 5-(((Trans-3-(3-cyclopropyl-4-(2-methoxycyclohexyl)-1H-pyrazol-1-yl)cyclobutyl)methyl)amino)-2-(2,6-dioxopiperidin-3-yl)isoindoline-1,3-dione (GS-646)

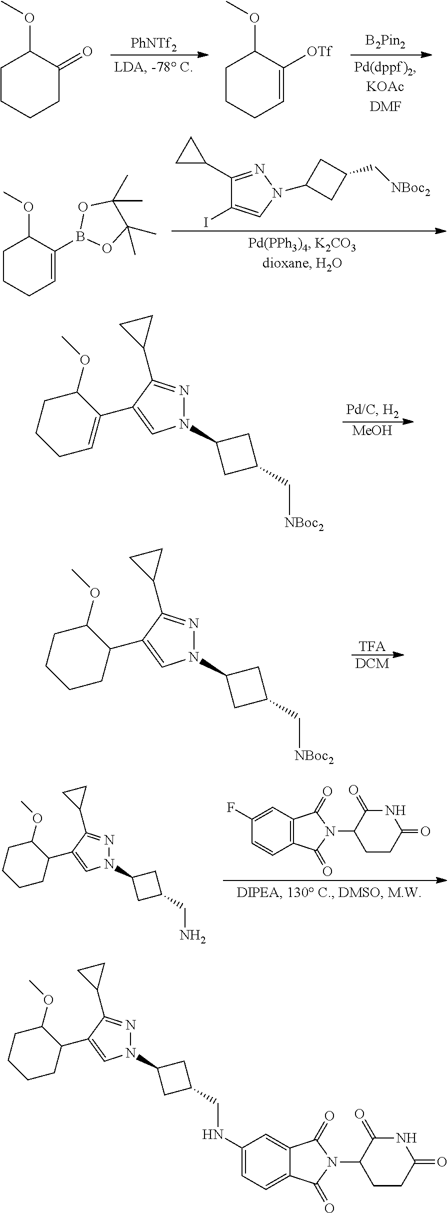

### Step 1. Synthesis of 6-methoxycyclohex-1-en-1-yl trifluoromethanesulfonate

To a solution of 2-methoxycyclohexan-1-one (1.5 g, 11.70 mmol) in dry THF (10 mL) was added LiHMDS (1 M, 15.21 mL) at −78 [symbol] , After the reaction was stirred at −78 [symbol] for 30 min, a solution of N-phenyl-bis(trifluoromethanesulfonimide) (4.60 g, 12.87 mmol) in dry THF (5.0 mL) was added dropwise. The reaction mixture was stirred at 25 [symbol] for 12 h. The mixture was quenched with H₂O (20 mL) and extracted with petroleum ether (30 mL×3). The combined organic phase was washed with brine, dried over anhydrous Na₂SO₄, filtered and evaporated under reduced pressure to provide the crude product (3 g, 98% yield) which was used directly in the next step without purification.

### Step 2. Synthesis of 2-(6-methoxycyclohex-1-en-1-yl)-4,4,5,5-tetramethyl-1,3,2-dioxaborolane

To a solution of 6-methoxycyclohex-1-en-1-yl trifluoromethanesulfonate (500 mg, 1.92 mmol), KOAc (565.7 mg, 5.76 mmol) and B₂Pin₂ (585.5 mg, 2.31 mmol) in DMF (10 mL) was added Pd(dppf)Cl₂ (140.6 mg, 0.192 mmol) at rt. The reaction mixture was stirred at 90 [symbol] for 12 h. The reaction was quenched with H₂O (20 mL) and extracted with ethyl acetate (20 mL×3). The combined organic layers were washed with saturated brine (20 mL), dried over anhydrous sodium sulfate, filtered and evaporated under reduced pressure. The resulting residue was purified by silica gel chromatography to provide the desired product (200 mg, 44% yield) as a yellow oil.

### Step 3. Synthesis of tert-butyl N-tert-butoxycarbonyl-N-((trans-3-(3-cyclopropyl-4-(6-methoxycyclohexen-1-yl)pyrazol-1-yl)cyclobutyl)methyl)carbamate

To a solution of 2-(6-methoxycyclohex-1-en-1-yl)-4,4,5,5-tetramethyl-1,3,2-dioxaborolane (200 mg, 0.84 mmol), tert-butyl N-tert-butoxycarbonyl-N-((trans-3-(3-cyclopropyl-4-iodo-pyrazol-1-yl)cyclobutyl)methyl)carbamate (434.6 mg, 0.84 mmol) and K₂CO₃ (232.2 mg, 1.68 mmol) in dioxane (5 mL) and H₂O (1 mL) was added Pd(dppf)Cl₂ (61.4 mg, 0.084 mmol) at rt. The reaction mixture was stirred at 90 [symbol] for 12 h. The reaction was quenched with H₂O (20 mL) and extracted with ethyl acetate (20 mL×3). The combined organic layers were washed with saturated brine (20 mL), dried over anhydrous sodium sulfate, filtered and evaporated under reduced pressure. The resulting residue was purified by silica gel chromatography to provide the desired product (100 mg, 24% yield) as a yellow oil. MS (ESI) m/z=502.9 [M+H]⁺.

### Step 4. Synthesis of tert-butyl N-tert-butoxycarbonyl-N-((trans-3-(3-cyclopropyl-4-(2-methoxycyclohexyl)pyrazol-1-yl)cyclobutyl)methyl)carbamate

To a solution tert-butyl N-tert-butoxycarbonyl-N-((trans-3-(3-cyclopropyl-4-(6-methoxycyclohexen-1-yl)pyrazol-1-yl)cyclobutyl)methyl)carbamate (100 mg, 0.2 mmol) in methanol (5 mL) was added 10% Pd/C (24.2 mg). The reaction mixture was purged three times with hydrogen and stirred under hydrogen atmosphere for 12 h. The mixture was filtered through Celite and the filtrate was concentrated under reduced pressure to provide the desired product (100 mg, 99% yield) as a yellow oil. This compound was used directly in the next step without further purification. MS (ESI) m/z=504.8 [M+H]⁺.

The remaining steps were performed according to the procedures for steps 3 to 4 of GS-641 to provide the desired product (0.9 mg, 2% yield over 2 steps) as a yellow solid. MS (ESI) m/z=560.6 [M+H]⁺.

### Example 8. 5-(((Trans-3-(3-cyclopropyl-4-(6-(3-hydroxyazetidin-1-yl)pyridin-2-yl)-1H-pyrazol-1-yl)cyclobutyl)methyl)amino)-2-(2,6-dioxopiperidin-3-yl)isoindoline-1,3-dione (GS-647)

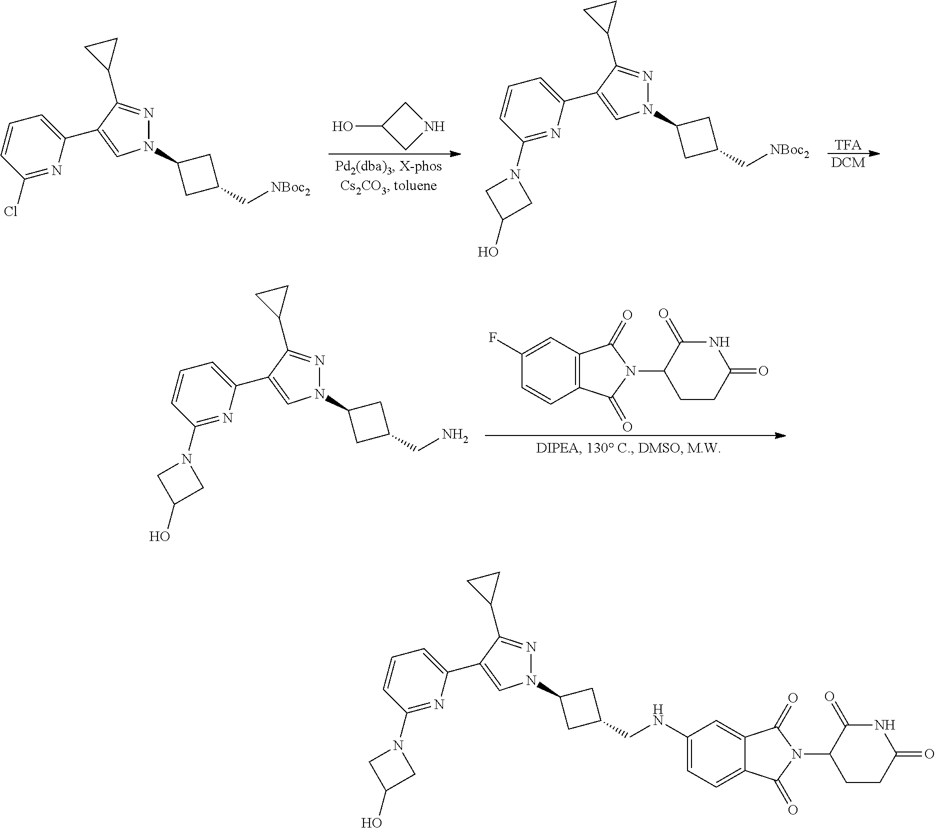

### Step 1. Synthesis of tert-butyl N-tert-butoxycarbonyl-N-((trans-3-(3-cyclopropyl-4-[6-(3-hydroxyazetidin-1-yl)-2-pyridyl)pyrazol-1-yl)cyclobutyl)methyl]carbamate

To a solution of azetidin-3-ol (43.6 mg, 0.40 mmol), tert-butyl N-tert-butoxycarbonyl-N-((trans-3-(4-(6-chloro-2-pyridyl)-3-cyclopropyl-pyrazol-1-yl)cyclobutyl)methyl)carbamate (100 mg, 0.20 mmol), XantPhos (23.0 mg, 0.04 mmol) and Cs₂CO₃ (194.3 mg, 0.60 mmol) in toluene (10 mL) was added Pd₂(dba)₃ (18.2 mg, 0.02 mmol) at rt. The reaction mixture was stirred at 100 [symbol] for 12 h. The reaction was quenched with H₂O (20 mL) and extracted with ethyl acetate (20 mL×3). The combined organic layers were washed with saturated brine (20 mL), dried over anhydrous sodium sulfate, filtered and evaporated under reduced pressure. The resulting residue was purified by silica gel chromatography to provide the desired product (13 mg, 12% yield) as a yellow oil. MS (ESI) m/z=540.6 [M+H]⁺.

The remaining steps were performed according to the procedures for steps 3 to 4 of GS-641 to provide the desired product (0.8 mg, 6% yield over 2 steps) as a yellow solid. MS (ESI) m/z=596.6 [M+H]⁺.

### Example 9. 5-(((Trans-3-(3-cyclopropyl-1H-pyrazolo[3,4-b]pyrazin-1-yl)cyclobutyl)methyl)amino)-2-(2,6-dioxopiperidin-3-yl)isoindoline-1,3-dione (GS-648)

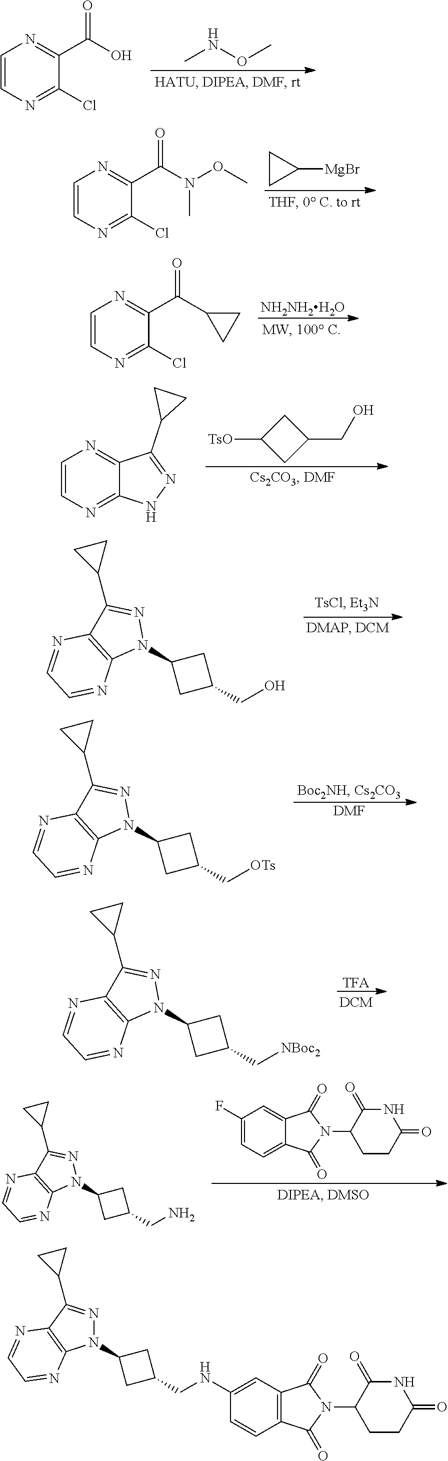

### Step 1. Synthesis of 3-chloro-N-methoxy-N-methylpyrazine-2-carboxamide

To a solution of 3-chloropyrazine-2-carboxylic acid (5.0 g, 31.6 mmol) in DMF (50 mL) were added HATU (14.4 g, 37.9 mmol), DIPEA (8.2 g, 63.3 mmol) and N,O-dimethylhydroxylamine (6.1 g, 63.3 mmol) at rt. After stirring at rt overnight, the reaction mixture was poured into water (200 mL) and extracted with ethyl acetate (100 mL×3). The combined organic layers were washed with saturated brine (50 mL), dried over anhydrous sodium sulfate, filtered and evaporated under reduced pressure. The residue was purified by column chromatography on silica-gel (petroleum ether/ethyl acetate=2:1) to provide the desired product (5.9 g, 93% yield) as a white solid. MS (ESI) m/z=202.2 [M+H]⁺.

### Step 2. Synthesis of (3-chloropyrazin-2-yl)(cyclopropyl)methanone

To a solution of 3-chloro-N-methoxy-N-methylpyrazine-2-carboxamide (5.9 g, 29.4 mmol) in THF (50 mL) was added a solution of cyclopropylmagnesium bromide (1.0 M, 35.2 mL, 35.2 mmol) in THF dropwise at 0 [symbol] . The reaction mixture was warmed to rt and stirred at the same temperature overnight. The reaction was quenched with a saturated solution of ammonium chloride and diluted with water (20 mL). The resulting mixture was stirred at rt until both layers were clear. The aqueous layer was separated and extracted with diethyl ether (20 mL×3). The combined organic layers were washed with saturated brine (20 mL), dried over anhydrous sodium sulfate, filtered and evaporated under reduced pressure. The resulting residue was purified by silica gel chromatography (petroleum ether/ethyl acetate=2:1) to provide the desired product (2.08 g, 39% yield) as a colorless oil. MS (ESI) m/z=183.1 [M+H]⁺.

### Step 3. Synthesis of 3-cyclopropyl-1H-pyrazolo[3,4-b]pyrazine

A solution of (3-chloropyrazin-2-yl)(cyclopropyl)methanone (2.08 g, 11.4 mmol) in hydrazine hydrate (20 mL) was heated at 100° C. for 1.5 h under microwave irradiation. After cooling down to rt, the reaction mixture was poured into water (20 mL) and extracted with dichloromethane (20 mL×4). The combined organic layers were washed with saturated brine (20 mL), dried over anhydrous sodium sulfate, filtered and evaporated under reduced pressure. The resulting residue was purified by silica gel chromatography (petroleum ether/ethyl acetate=1:1) to provide the desired product (1.09 g, 60% yield) as a white solid. ¹H NMR (400 MHz, DMSO-d₆) δ 13.55 (s, 1H), 8.53 (s, 2H), 2.39-2.32 (m, 1H), 1.21-1.17 (m, 2H), 1.08-1.03 (m, 2H). MS (ESI) m/z=161.1 [M+H]⁺.

### Step 4. Synthesis of (trans-3-(3-cyclopropyl-1H-pyrazolo[3,4-b]pyrazin-1-yl)cyclobutyl)methanol

To a solution of 3-cyclopropyl-1H-pyrazolo[3,4-b]pyrazine (150 mg, 0.94 mmol) and 3-(hydroxymethyl)cyclobutyl 4-methylbenzenesulfonate (432 mg, 1.96 mmol) in DMF (5 mL) was added Cs₂CO₃ (610.6 mg, 1.87 mmol) at rt. The reaction mixture was stirred at 100 [symbol] for 4 h. After cooling down to it, the reaction was quenched with H₂O (20 mL) and extracted with EtOAc (30 mL×3). The combined organic phase was washed with brine, dried over anhydrous Na₂SO₄, filtered, and concentrated under vacuum. The resulting residue was purified by silica gel column chromatography to provide two isomers. The desired trans-isomer (90 mg, 39% yield) was got as a yellow solid. MS (ESI) m/z=245.5 [M+H]⁺.

The remaining steps were performed according to the procedures for steps 5 to 6 GS-643 and steps 3 to 4 of GS-641 to provide the desired product (4.2 mg, 10% yield over 4 steps) as a yellow solid. MS (ESI) m/z=500.5 [M+H]⁺.

### Example 10. 5-(((trans-3-(3-Cyclopropyl-4-(1-methyl-1H-pyrazolo[3,4-c]pyridin-7-yl)-1H-pyrazol-1-yl)cyclobutyl)methyl)amino)-2-(2,6-dioxopiperidin-3-yl)isoindoline-1,3-dione (GS-649)

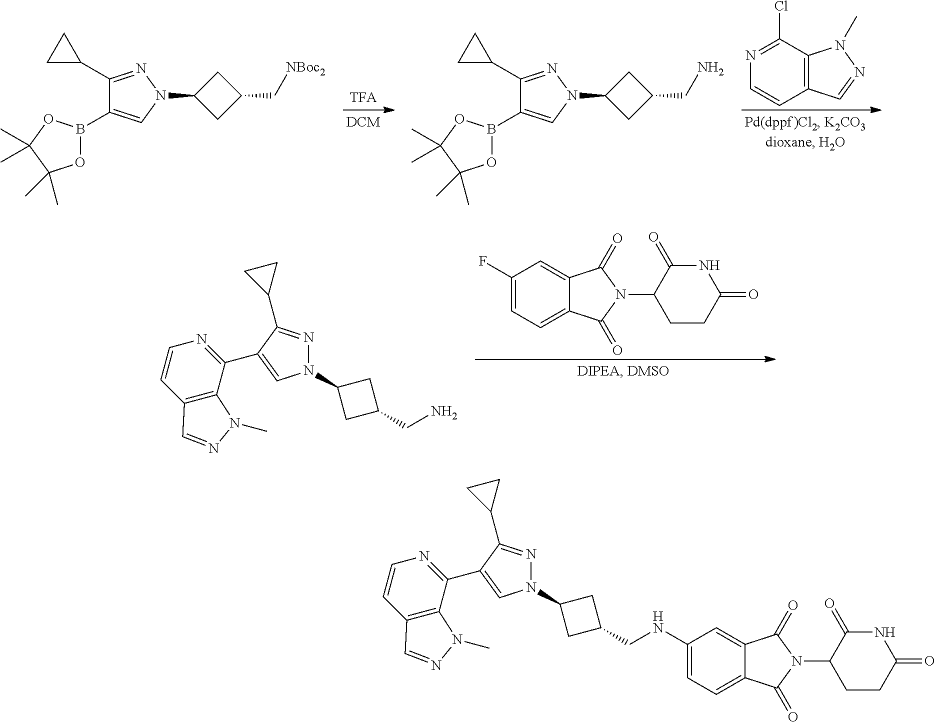

### Step 1. Synthesis of (trans-3-(3-cyclopropyl-4-(4,4,5,5-tetramethyl-1,3,2-dioxaborolan-2-y)-1H-pyrazol-1-yl)cyclobutyl)methanamine

A solution of tert-butyl N-tert-butoxycarbonyl-N-(trans-3-(3-cyclopropyl-4-(4,4,5,5-tetra methyl-1,3,2-dioxaborolan-2-yl)pyrazol-1-yl)cyclobutyl)methyl)carbamate (180 mg, 0.348 mmol) in TFA (1.5 mL) and dichloromethane (1.5 mL) was stirred at rt for 12 h. The reaction mixture was concentrated and purified by reverse-phase chromatography to provide the desired product (50 mg, 45% yield) as a colorless oil. MS (ESI) m/z=318.3 [M+H]⁺.

### Step 2. Synthesis of (trans-3-(3-cyclopropyl-4-(1-methyl-1H-pyrazolo[3,4-c]pyridin-7-yl)-1H-pyrazol-1-yl)cyclobutyl)methanamine

A mixture of 7-chloro-1-methyl-pyrazolo[3,4-c]pyridine (30 mg, 0.18 mmol), (trans-3-(3-cyclopropyl-4-(4,4,5,5-tetramethyl-1,3,2-dioxaborolan-2-yl)-1H-pyrazol-1-yl)cyclobutyl)methanamine (56.8 mg, 0.18 mmol), K₂CO₃ (74.1 mg, 0.537 mmol) and Pd(dppf)Cl₂ (26.2 mg, 0.0358 mmol) in dioxane (5 mL) and water (1 mL) was stirred at 100 [symbol] for 1 h under microwave irradiation. After cooling down to rt, the mixture was concentrated and purified by reverse-phase chromatography to provide the desired product (20 mg, 35% yield) as a colorless oil. MS (ESI) m/z=323.4 [M+H]⁺.

### Step 3. Synthesis of 5-(((trans-3-(3-Cyclopropyl-4-(1-methyl-1H-pyrazolo[3,4-c]pyridin-7-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(1-methyl-1H-pyrazolo[3,4-c]pyridin-7-yl)-1H-pyrazol-1-yl)cyclobutyl)methanamine (20 mg, 0.062 mmol) and 2-(2,6-dioxopiperidin-3-yl)-5-fluoroisoindoline-1,3-dione (25.7 mg, 0.093 mmol) in DMSO (1 mL) was added DIPEA (32.0 mg, 0.25 mmol). The mixture was stirred at 125 [symbol] for 45 min under microwave irradiation. After cooling down to rt, the mixture was purified by reverse-phase chromatography to provide the desired product (6.5 mg, 18% yield) as a yellow solid. MS (ESI) m/z=579.8 [M+H]⁺.

### Example 11. 5-(((trans-3-(3-Cyclopropyl-4-(2-(dimethylamino)phenyl)-1H-pyrazol-1-yl)cyclobutyl)methyl)amino)-2-(2,6-dioxopiperidin-3-yl)isoindoline-1,3-dione (GS-650)

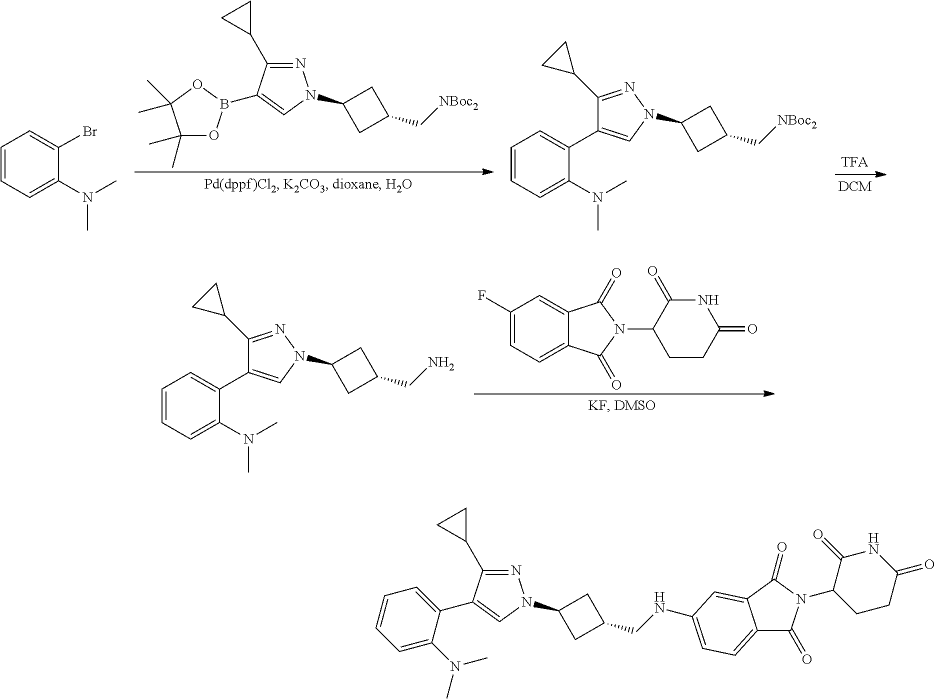

### Step 1. Synthesis of tert-butyl N-tert-butoxycarbonyl-N-((3-(3-cyclopropyl-4-(2-(dimethylamino)phenyl)pyrazol-1-yl)cyclobutyl)methyl)carbamate

To a solution of 2-bromo-N,N-dimethyl-aniline (50 mg, 0.25 mmol) in dioxane (8 mL) and H₂O (2 mL) were added K₂CO₃ (51.7 mg, 0.38 mmol) and Pd(dppf)Cl2 (17.8 mg, 0.025 mmol) at rt. The reaction mixture was stirred at 90 [symbol] for 1 h. After cooling down to rt, the reaction was quenched with H₂O (10 mL) and extracted with EtOAc (3×20 mL). The combined organic layers were dried over anhydrous sodium sulfate, filtered, and concentrated under reduced pressure. The resulting residue was purified by reverse-phase chromatography to provide the desired product (50 mg, 40% yield) as a light-yellow solid. MS (ESI) m/z=511.5 [M+H]⁺.

The remaining steps were performed according to the procedures for steps 2 to 3 of GS-642 (5.0 mg, 13% yield over 2 steps) as a light-yellow solid. MS (ESI) m/z=567.8 [M+H]⁺.

### Example 12. 3-(5-(((trans-3-(3-Cyclopropyl-4-(quinoxalin-2-yl)-1H-pyrazol-1-yl)cyclobutyl)methyl)amino)-1-oxoisoindolin-2-yl)piperidine-2,6-dione (GS-651)

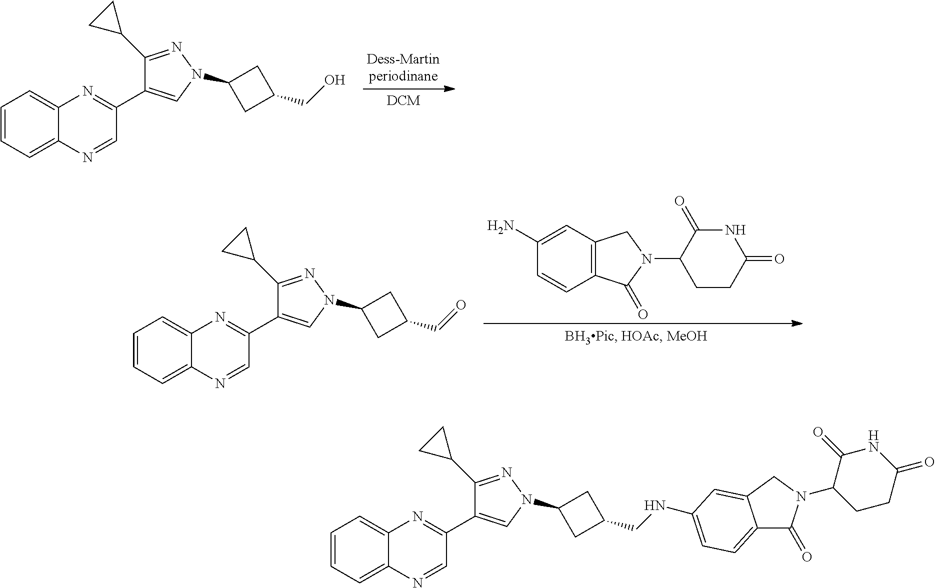 [SMILES: OC[C@H]1C[C@H](n2cc(-c3cnc4ccccc4n3)c(C3CC3)n2)C1 ; O=C[C@H]1C[C@H](n2cc(-c3cnc4ccccc4n3)c(C3CC3)n2)C1 ; Nc1ccc2c(c1)CN(C1CCC(=O)NC1=O)C2=O ; O=C1CCC(N2Cc3cc(NC[C@H]4C[C@H](n5cc(-c6cnc7ccccc7n6)c(C6CC6)n5)C4)ccc3C2=O)C(=O)N1]

### Step 1. Synthesis of trans-3-(3-cyclopropyl-4-(quinoxalin-2-yl)-1H-pyrazol-1-yl)cyclobutane-1-carbaldehyde

To a solution of (trans-3-(3-cyclopropyl-4-(quinoxalin-2-yl)-1H-pyrazol-1-yl)cyclobutyl)methanol (130 mg, 0.40 mmol) in dichloromethane (5 mL) was added Dess-Martin periodinane (516.3 mg, 1.22 mmol). The reaction mixture was stirred at 25 [symbol] for 2 h, before it was quenched with H₂O (20 mL) and extracted with dichloromethane (20 mL×3). The combined organic phase was washed with brine (20 mL) and dried over anhydrous Na₂SO₄, filtered and concentrated under reduced pressure. The residue was purified by silica gel chromatography to provide the desired product (80 mg, 62% yield) as a yellow oil. MS (ESI) m/z=319.4 [M+H]⁺.

### Step 2. Synthesis of 3-(5-(((trans-3-(3-cyclopropyl-4-(quinoxalin-2-yl)-1H-pyrazol-1-yl)cyclobutyl)methyl)amino)-1-oxoisoindolin-2-yl)piperidine-2,6-dione

To a solution of trans-3-(3-cyclopropyl-4-(quinoxalin-2-yl)-1H-pyrazol-1-yl)cyclobutane-1-carbaldehyde (40 mg, 0.12 mmol), two drops of acetic acid and 3-(5-amino-1-oxoisoindolin-2-yl)piperidine-2,6-dione (32.6 mg, 0.12 mmol) in methanol (2 mL) was added 2-picoline borane complex (20.2 mg, 0.19 mmol). The reaction mixture was stirred at 25 [symbol] for 12 h, before it was quenched with H₂O (20 mL) and extracted with dichloromethane (20 mL×3). The combined organic phase was washed with brine (20 mL), dried over anhydrous Na₂SO₄, filtered and concentrated under reduced pressure. The residue was purified by silica gel chromatography to provide the desired product (3.4 mg, 5% yield) as a yellow solid. MS (ESI) m/z=562.7 [M+H]⁺.

### Example 13. 5-(((Trans-3-(3-cyclopropyl-4-(2-(piperazin-1-yl)phenyl)-1H-pyrazol-1-yl)cyclobutyl)methyl)amino)-2-(2,6-dioxopiperidin-3-yl)isoindoline-1,3-dione (GS-652)

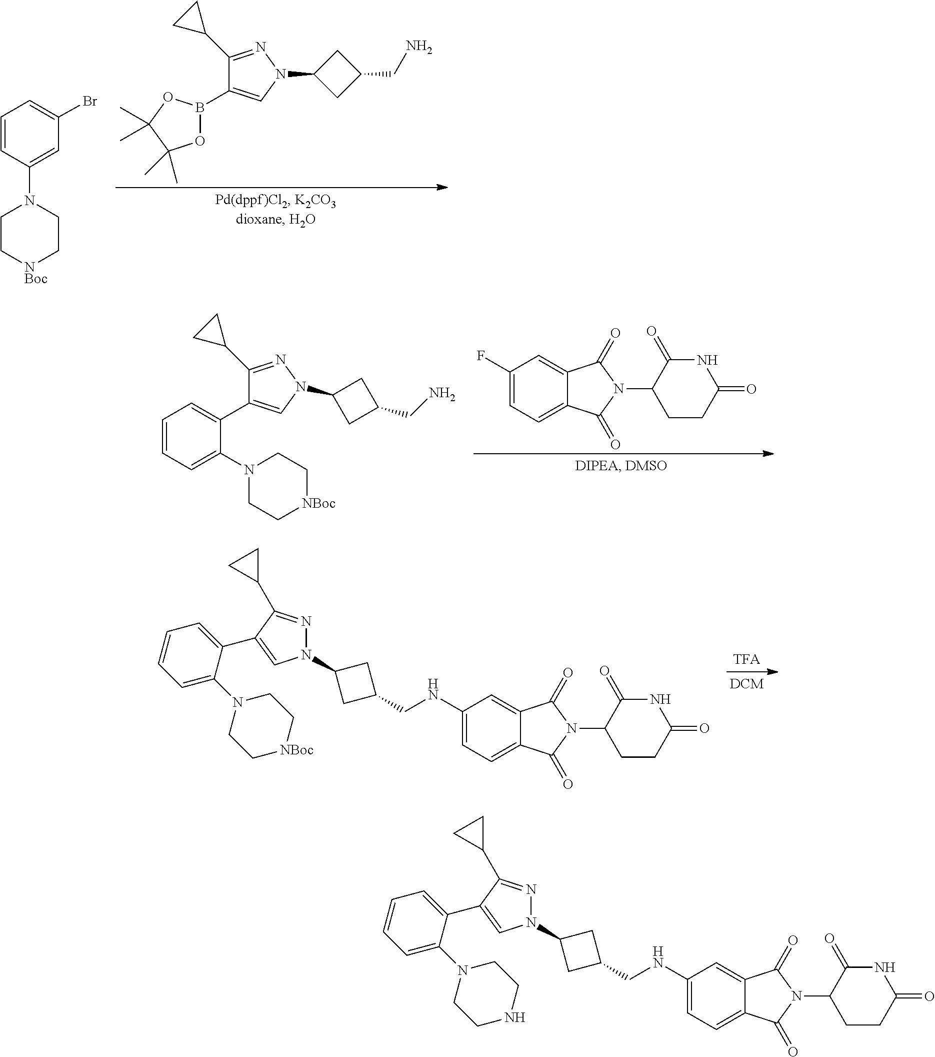

GS-652 was synthesized following the procedures for steps 1 to 3 of GS-649 (6.5 mg, 7% yield over 3 steps) as a yellow solid. MS (ESI) m/z=608.7 [M+H]⁺.

### Example 14. 5-(((Trans-3-(4-(3-(azetidin-1-yl)phenyl)-3-cyclopropyl-1H-pyrazol-1-yl)cyclobutyl)methyl)amino)-2-(2,6-dioxopiperidin-3-yl)isoindoline-1,3-dione (GS-653)

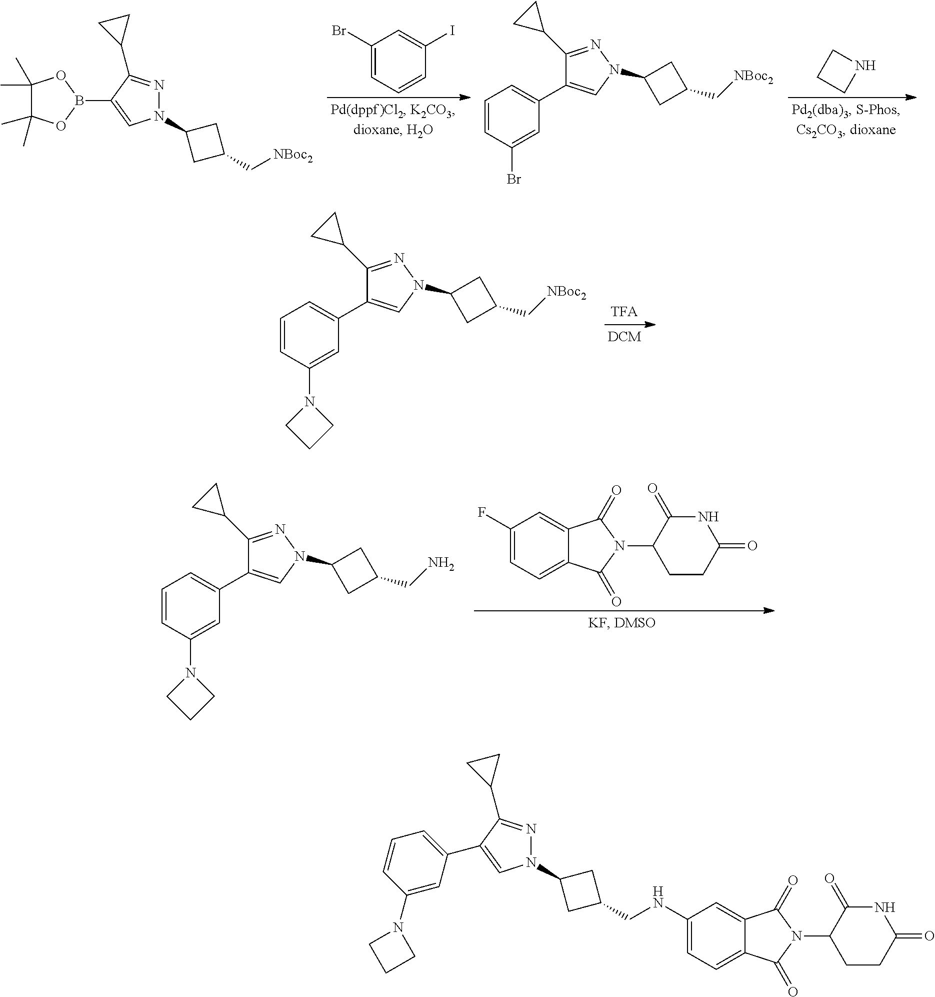

### Step 1. Synthesis of tert-butyl N-((3-(4-(3-bromophenyl)-3-cyclopropyl-pyrazol-1-yl)cyclobutyl)methyl)-N-tert-butoxycarbonyl-carbamate

To a mixture of 1-bromo-3-iodo-benzene (50 mg, 0.177 mmol) and tert-butyl N-tert-butoxycarbonyl-N-((3-(3-cyclopropyl-4-(4,4,5,5-tetramethyl-1,3,2-dioxaborolan-2-yl)pyrazol-1-yl)cyclobutyl)methyl)carbamate (91.5 mg, 0.177 mmol) in dioxane (3 mL) and H₂O (1 mL) were added Pd(dppf)Cl₂ (25.8 mg, 0.035 mmol) and K₂CO₃ (73.2 mg, 0.53 mmol) at rt. The reaction mixture was stirred at 90 [symbol] for 1 h. After cooling down to rt, the reaction was quenched with H₂O (10 mL) and extracted with EtOAc (3×20 mL). The combined organic layers were dried over anhydrous sodium sulfate, filtered, and concentrated under reduced pressure. The resulting residue was purified by reverse-phase chromatography to provide the desired product (50 mg, 52% yield) as a light-yellow solid. MS (ESI) m/z=546.5 [M+H]⁺.

### Step 2. Synthesis of tert-butyl N-((3-(4-(3-(azetidin-1-v)phenyl)-3-cyclopropyl-pyrazol-1-yl)cyclobutyl)methyl)-N-tert-butoxycarbonyl-carbamate

To a solution of tert-butyl N-((3-(4-(3-bromophenyl)-3-cyclopropyl-pyrazol-1-yl)cyclobutyl)methyl)-N-tert-butoxycarbonyl-carbamate (50 mg, 0.092 mmol) in dioxane (3 mL) were added S-Phos (37.60 mg, 0.092 mmol), Pd₂(dba)₃ (8.4 mg, 0.0092 mmol) and Cs₂CO₃ (89.2 mg, 0.27 mmol) at rt. The reaction mixture was stirred at 90 [symbol] for 3 h. After cooling down to rt, the reaction was quenched with H₂O (10 mL) and extracted with EtOAc (3×20 mL). The combined organic layers were dried over anhydrous sodium sulfate, filtered, and concentrated under reduced pressure. The resulting residue was purified by reverse-phase chromatography to provide the desired product (30 mg, 63% yield) as a yellow solid. MS (ESI) m/z=523.5 [M+H]⁺.

The remaining steps were performed according to the procedures for steps 2 to 3 of GS-642 (3 mg, 11% yield over 2 steps) as a light-yellow solid. MS (ESI) m/z=579.7 [M+H]⁺.

### Example 15. 5-(((Trans-3-(3-cyclopropyl-4-(2-(piperidin-4-yl)phenyl)-1H-pyrazol-1-yl)cyclobutyl)methyl)amino)-2-(2,6-dioxopiperidin-3-yl)isoindoline-1,3-dione (GS-654)

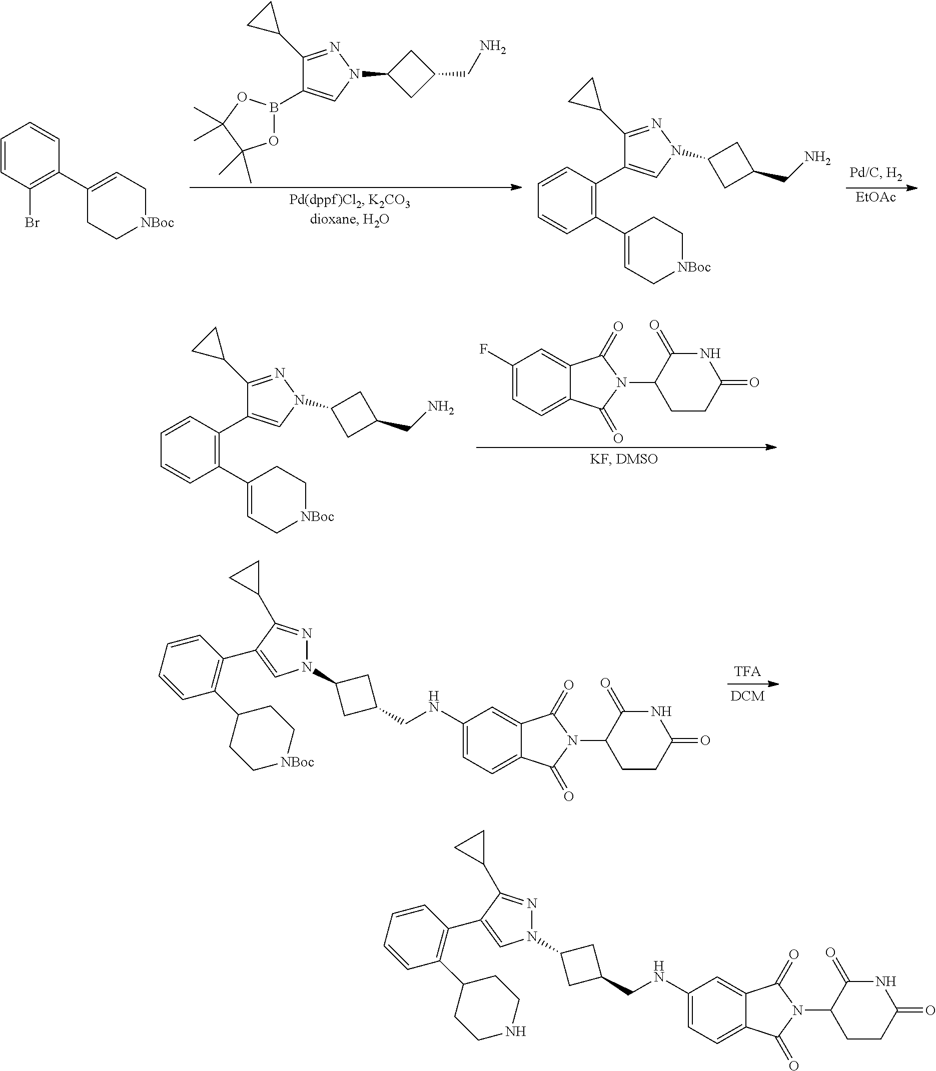

### Step 1. Synthesis of tert-butyl 4-(2-(1-(trans-3-(aminomethyl)cyclobutyl)-3-cyclopropyl-1H-pyrazol-4-yl)phenyl)-3,6-dihydropyridine-1 (2H)-carboxylate

The title compound was synthesized following the step 2 of GS-649 (30 mg, 45% yield) as a yellow solid. MS (ESI) m/z=449.6 [M+H]⁺.

### Step 2. Synthesis of tert-butyl 4-(2-(1-(trans-3-(aminomethyl)cyclobutyl)-3-cyclopropyl-1H-pyrazol-4-yl)phenyl)piperidine-1-carboxylate

To a solution of tert-butyl 4-(2-(1-(trans-3-(aminomethyl)cyclobutyl)-3-cyclopropyl-1H-pyrazol-4-yl)phenyl)-3,6-dihydropyridine-1 (2H)-carboxylate (30 mg, 0.067 mmol) in ethyl acetate (3 mL) was added Pd/C (12.2 mg, 10% wt) at rt. The reaction mixture was purged with hydrogen three times and stirred at rt for 20 min under hydrogen balloon. The mixture was filtered through Celite and the filter cake was washed with EtOAc (10 mL). The filtrate was concentrated under reduced pressure. The residue was purified by reverse-phase chromatography to provide the desired product (20 mg, 66% yield) as a yellow solid.

The remaining steps were performed according to the procedures for steps 2 to 3 of GS-642 (3 mg, 110% yield over 2 steps) as a yellow solid. MS (ESI) m/z=607.7 [M+H]⁺.

### Example 16. 5-(((Trans-3-(3-cyclopropyl-4-(7-(1-methylazetidin-3-yl)quinoxalin-2-yl)-1H-pyrazol-1-yl)cyclobutyl)methyl)amino)-2-(2,6-dioxopiperidin-3-yl)isoindoline-1,3-dione (GS-655)

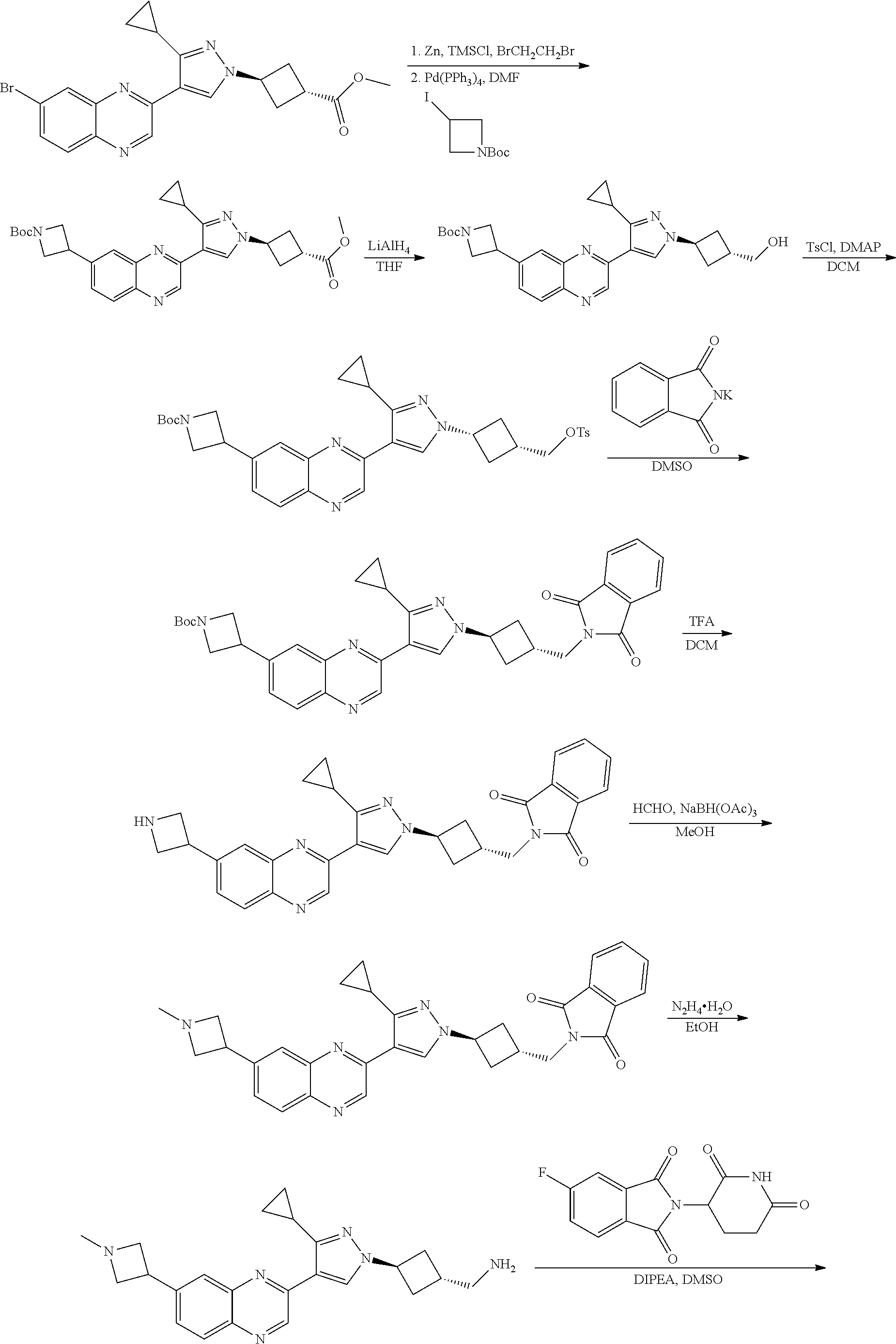

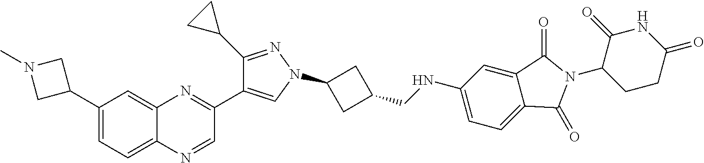 [SMILES: CN1CC(c2ccc3ncc(-c4cn([C@H]5C[C@H](CNc6ccc7c(c6)C(=O)N(C6CCC(=O)NC6=O)C7=O)C5)nc4C4CC4)nc3c2)C1]

### Step 1. Synthesis of tert-butyl 3-(3-(3-cyclopropyl-1-(trans-3-(methoxycarbonyl)cyclobutyl)-1H-pyrazol-4-yl)quinoxalin-6-yl)azetidine-1-carboxylate

To a suspension of Zn dust (382 mg, 5.85 mmol) in dry DMF (20 mL) was added 1,2-dibromoethane (45 mg, 0.23 mmol) at rt under N₂ atmosphere. After stirring at 80° C. for 10 min, the reaction mixture was cooled to rt. Trimethylsilyl chloride (25 mg, 0.23 mmol) in DMF (2 mL) was added dropwise. The reaction mixture was stirred at rt for 45 min, before a solution of tert-butyl 3-iodoazetidine-1-carboxylate (1.6 g, 5.85 mmol) in DMF (5 mL) was added dropwise. The resulting mixture was stirred at 45 [symbol] for another 2 h, before a solution of methyl trans-3-(4-(7-bromoquinoxalin-2-yl)-3-cyclopropyl-1H-pyrazol-1-yl)cyclobutane-1-carboxylate (500 mg, 1.17 mmol) and Pd(PPh₃)₄ (135 mg, 0.11 mmol) in DMF (20 mL) was added. After stirring at 70 [symbol] for 16 h, the reaction mixture was diluted with water (200 mL) and extracted with ethyl acetate (3×150 mL). The combined organic layers were washed with brine (3×50 mL), dried over Na₂SO₄, filtered and concentrated. The residue was purified by column chromatography on silica-gel to provide the desired product (270 mg, 46% yield) as a brown solid. MS (ESI) m/z=504.7 [M+H]⁺.

### Step 2. Synthesis of tert-butyl 3-(3-(3-cyclopropyl-1-(trans-3-(hydroxymethyl)cyclobutyl)-1H-pyrazol-4-yl)quinoxalin-6-yl)azetidine-1-carboxylate

To a solution of tert-butyl 3-(3-(3-cyclopropyl-1-(trans-3-(methoxycarbonyl)cyclobutyl)-1H-pyrazol-4-yl)quinoxalin-6-yl)azetidine-1-carboxylate (130 mg, 0.26 mmol) in THF (20 mL) was added LiAlH₄ (26 mg, 0.7 mmol) at 0° C. under N₂. After the reaction mixture was stirred at rt for 1 h, H₂O (0.1 mL) and anhydrous Na₂SO₄ were added into the mixture. The precipitate was filtered and the filtrate was concentrated to provide the desired crude product (110 mg, 89% yield) as a yellow oil. MS (ESI) m/z=476.6 [M+H]⁺.

### Step 3. Synthesis of tert-butyl 3-(3-(3-cyclopropyl-1-(trans-3-((tosyloxy)methyl)cyclobutyl)-1H-pyrazol-4-yl)quinoxalin-6-yl)azetidine-1-carboxylate

To a solution of tert-butyl 3-(3-(3-cyclopropyl-1-(trans-3-(hydroxymethyl)cyclobutyl)-1H-pyrazol-4-yl)quinoxalin-6-yl)azetidine-1-carboxylate (110 mg, 0.23 mmol) in dichloromethane (20 mL) were added TsCl (88 mg, 0.46 mmol) and DMAP (56 mg, 0.46 mmol) at 0 [symbol] . The reaction mixture was stirred at rt for 16 h, before it was poured into water (20 mL) and extracted with dichloromethane (3×20 mL). The combined organic layers were washed with saturated brine, dried over anhydrous sodium sulfate, filtered and evaporated under reduced pressure. The resulting residue was purified by silica gel flash chromatography to provide the desired product (130 mg, 89% yield) as a white solid. MS (ESI) m/z=630.6 [M+H]⁺.

### Step 4. Synthesis of tert-butyl 3-(3-(3-cyclopropyl-1-(trans-3-((1,3-dioxoisoindolin-2-yl)methyl)cyclobutyl)-1H-pyrazol-4-yl)quinoxalin-6-yl)azetidine-1-carboxylate

To a solution of tert-butyl 3-(3-(3-cyclopropyl-1-(trans-3-((tosyloxy)methyl)cyclobutyl)-1H-pyrazol-4-yl)quinoxalin-6-yl)azetidine-1-carboxylate (130 mg, 0.21 mmol) in DMSO (10 mL) was added potassium 1,3-dioxoisoindolin-2-ide (76 mg, 0.42 mmol) at rt. The reaction mixture was stirred at 80° C. for 2 h. After cooling down to rt, the reaction mixture was poured into water (20 mL) and extracted with ethyl acetate (3×20 mL). The combined organic layers were washed with saturated brine, dried over anhydrous sodium sulfate, filtered and evaporated under reduced pressure. The resulting residue was purified by silica gel flash chromatography to provide the desired product (110 mg, 88% yield) as a colorless oil. MS (ESI) m/z=605.7 [M+H]⁺.

### Step 5. Synthesis of 2-((trans-3-(4-(7-(azetidin-3-yl)quinoxalin-2-yl)-3-cyclopropyl-1H-pyrazol-1-yl)cyclobutyl)methyl)isoindoline-1,3-dione

To a solution of tert-butyl 3-(3-(3-cyclopropyl-1-(trans-3-((1,3-dioxoisoindolin-2-yl)methyl)cyclobutyl)-1H-pyrazol-4-yl)quinoxalin-6-yl)azetidine-1-carboxylate (110 mg, 0.18 mmol) in DCM (2 mL) was added TFA (1 mL) at 0 [symbol] . After stirring at rt for 1 h, the reaction mixture was concentrated under vacuum to provide the crude product which was used in the next step directly without further purification. MS (ESI) m/z=505.5 [M+H]⁺.

### Step 6. Synthesis of 2-((trans-3-(3-cyclopropyl-4-(7-(1-methylazetidin-3-yl)quinoxalin-2-yl)-1H-pyrazol-1-yl)cyclobutyl)methyl)isoindoline-1,3-dione

To a solution of 2-((trans-3-(4-(7-(azetidin-3-yl)quinoxalin-2-yl)-3-cyclopropyl-1H-pyrazol-1-yl)cyclobutyl)methyl)isoindoline-1,3-dione (90 mg, 0.18 mmol) in MeOH (10 mL) and AcOH (1 mL) were added formaldehyde solution (37 wt. % in H₂O, 328 mg, 3.57 mmol) and NaBH(OAc)₃ (756.0 mg, 3.57 mmol). After the reaction mixture was stirred at rt for 3 days, it was concentrated under reduced pressure. The residue was purified by reverse-phase chromatography to provide the desired product (85 mg, 91% yield) as a yellow solid. MS (ESI) m/z=519.6 [M+H]⁺.

### Step 7. Synthesis of (trans-3-(3-cyclopropyl-4-(7-(1-methylazetidin-3-yl)quinoxalin-2-yl)-1H-pyrazol-1-yl)cyclobutyl)methanamine

To a solution of 2-((trans-3-(3-cyclopropyl-4-(7-(1-methylazetidin-3-yl)quinoxalin-2-yl)-1H-pyrazol-1-yl)cyclobutyl)methyl)isoindoline-1,3-dione (85 mg, 0.16 mmol) in EtOH (10 mL) was added hydrazine hydrate (164 mg, 3.28 mmol) at rt. After the reaction mixture was stirred at 50 [symbol] for 2 h, it was concentrated under reduced pressure. The residue was purified by reverse-phase chromatography to provide the desired product (45 mg, 710% yield) as a yellow solid. MS (ESI) m/z=389.4 [M+H]⁺.

### Step 8. Synthesis of 5-(((trans-3-(3-cyclopropyl-4-(7-(1-methylazetidin-3-yl)quinoxalin-2-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(7-(1-methylazetidin-3-yl)quinoxalin-2-yl)-1H-pyrazol-1-yl)cyclobutyl)methanamine (10 mg, 0.02 mmol) in DMSO (2 mL) were added 2-(2,6-dioxo-3-piperidyl)-5-fluoro-isoindoline-1,3-dione (14 mg, 0.04 mmol) and DIPEA (33 mg, 0.25 mmol). The reaction mixture was stirred at 130° C. for 30 min under microwave irradiation. After cooling down to rt, the mixture was purified by reverse-phase chromatography to provide the desired product (1.2 mg, 6% yield) as a yellow solid. MS (ESI) m/z=645.7 [M+H]⁺.

### Example 17. 5-(((Trans-3-(3-(difluoromethyl)-4-(1-isopropyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-656)

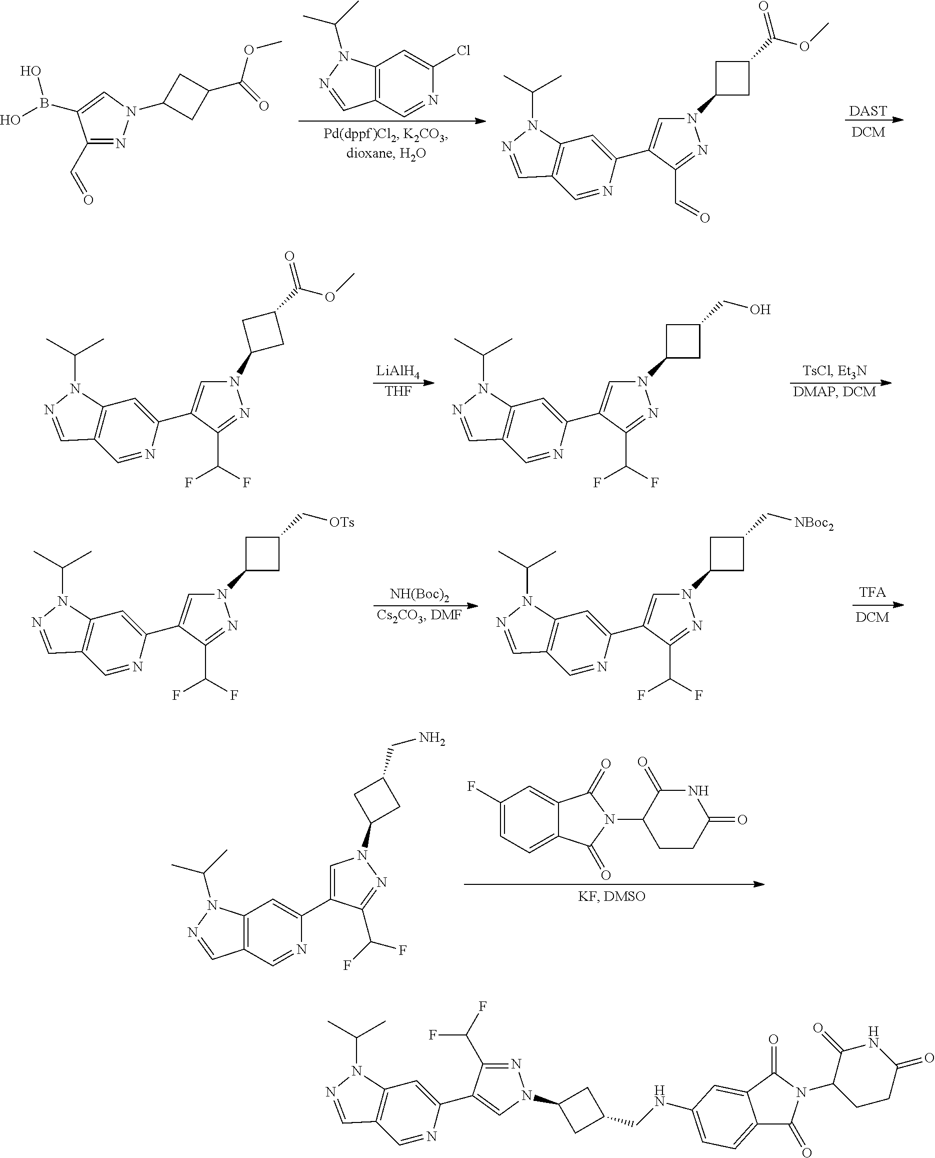

GS-656 was synthesized following the procedures for steps 1 to 3 of GS-677 and steps 5 to 8 of GS-643 (2 mg, 1% yield over 7 steps) as a yellow solid. MS (ESI) m/z=617.7 [M+H]⁺.

### Example 18. 5-(((Trans-3-(3-(difluoromethyl)-4-(1-methyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-657)

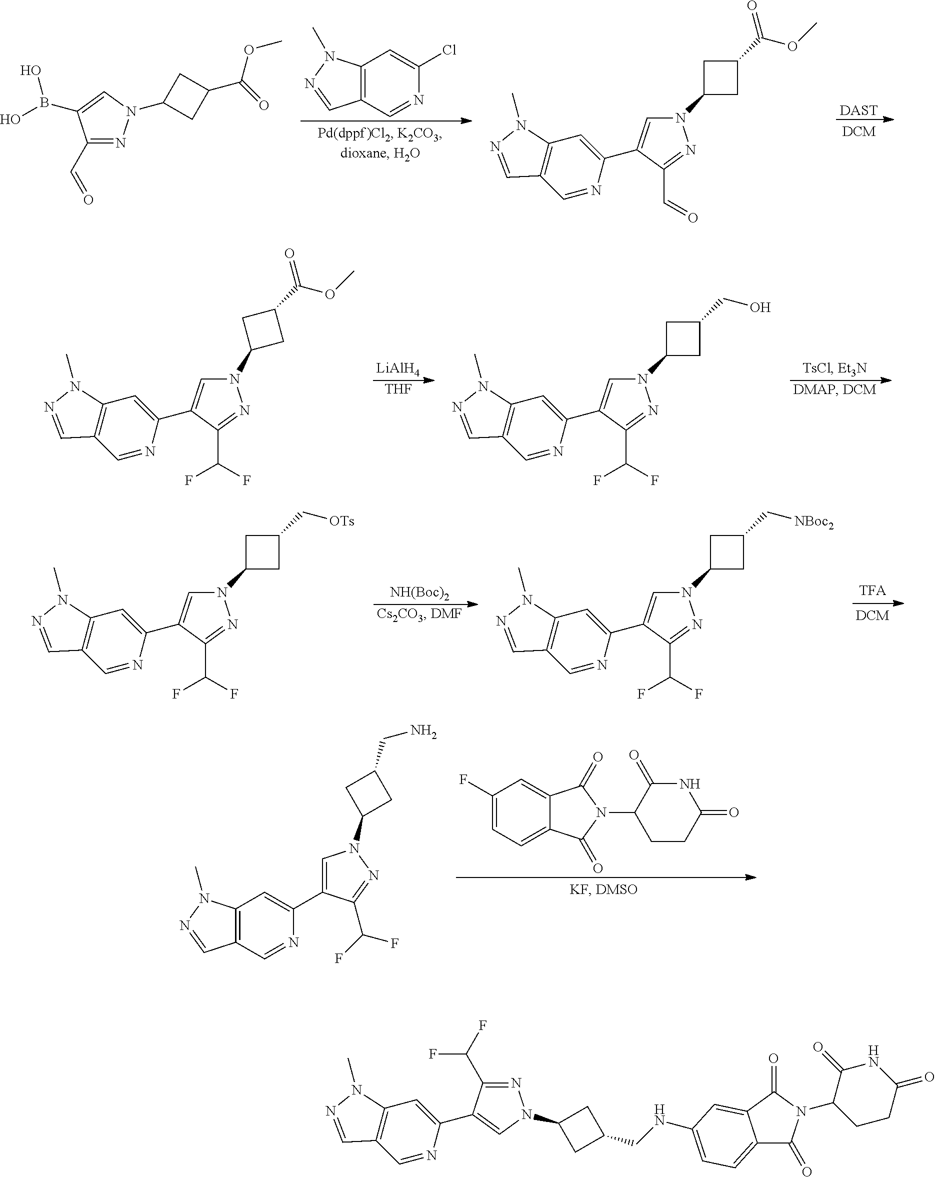

GS-657 was synthesized following the procedures for steps 1 to 3 of GS-677 and steps 5 to 8 of GS-643 (4 mg, 3% yield over 7 steps) as a yellow solid. MS (ESI) m/z=589.7 [M+H]⁺.

### Example 19. 5-(((Trans-3-(3-cyclopropyl-4-(1-methyl-1H-imidazo[4,5-c]pyridin-6-yl)-1H-pyrazol-1-yl)cyclobutyl)methyl)amino)-2-(2,6-dioxopiperidin-3-yl)isoindoline-1,3-dione (GS-658)

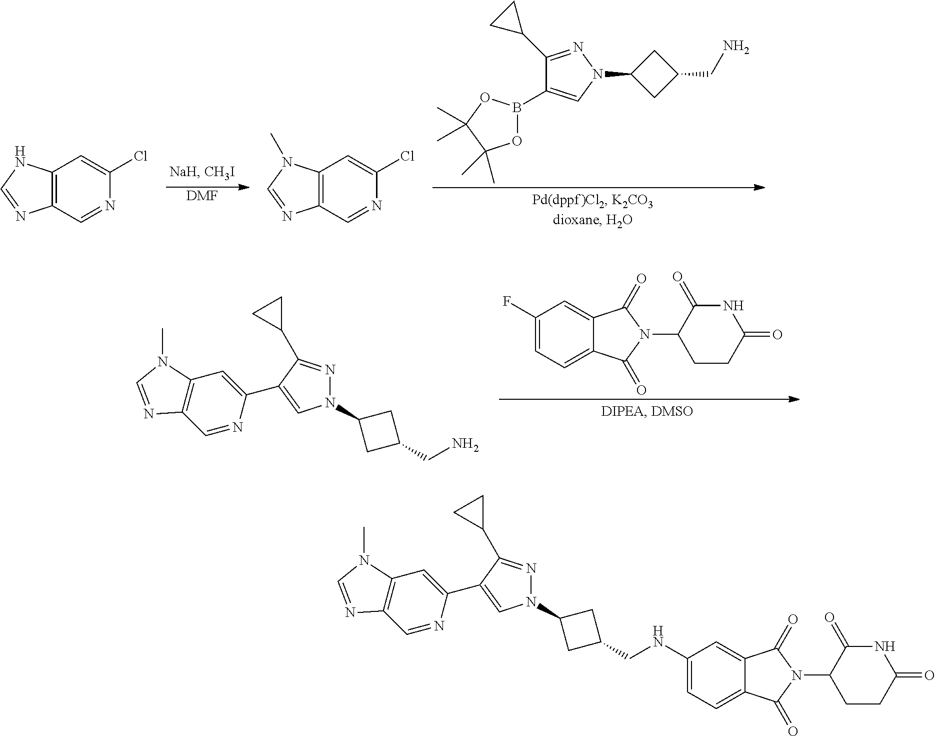

### Step 1. Synthesis of 6-chloro-1-methyl-1H-imidazo[4,5-c]pyridine

To a stirred solution of 6-chloro-1H-imidazo[4,5-c]pyridine (200 mg, 1.30 mmol) and NaH (93.7 mg, 3.91 mmol) in DMF (4 mL) was added CH₃I (554.5 mg, 3.91 mmol) dropwise at rt. The reaction mixture was stirred at rt for 1 h, before it was quenched with ice water (20 mL) and extracted with ethyl acetate (3×20 mL). The combined organic layers were washed with saturated brine, dried over anhydrous sodium sulfate, filtered and concentrated under reduced pressure. The resulting residue was purified by prep-HPLC to provide the desired product (75 mg, 34% yield) as a white solid. MS (ESI) m/z=168.1 [M+H]⁺.

The remaining steps were performed according to the procedures for steps 2 to 3 of GS-649 to provide the desired product (6.5 mg, 6% yield over 2 steps) as a yellow solid. MS (ESI) m/z=579.5 [M+H]⁺.

### Example 20. 5-(((Trans-3-(3-cyclopropyl-4-(7-(3-hydroxypyrrolidin-1-yl)quinoxalin-2-yl)-1H-pyrazol-1-yl)cyclobutyl)methyl)amino)-2-(2,6-dioxopiperidin-3-yl)isoindoline-1,3-dione (GS-659)

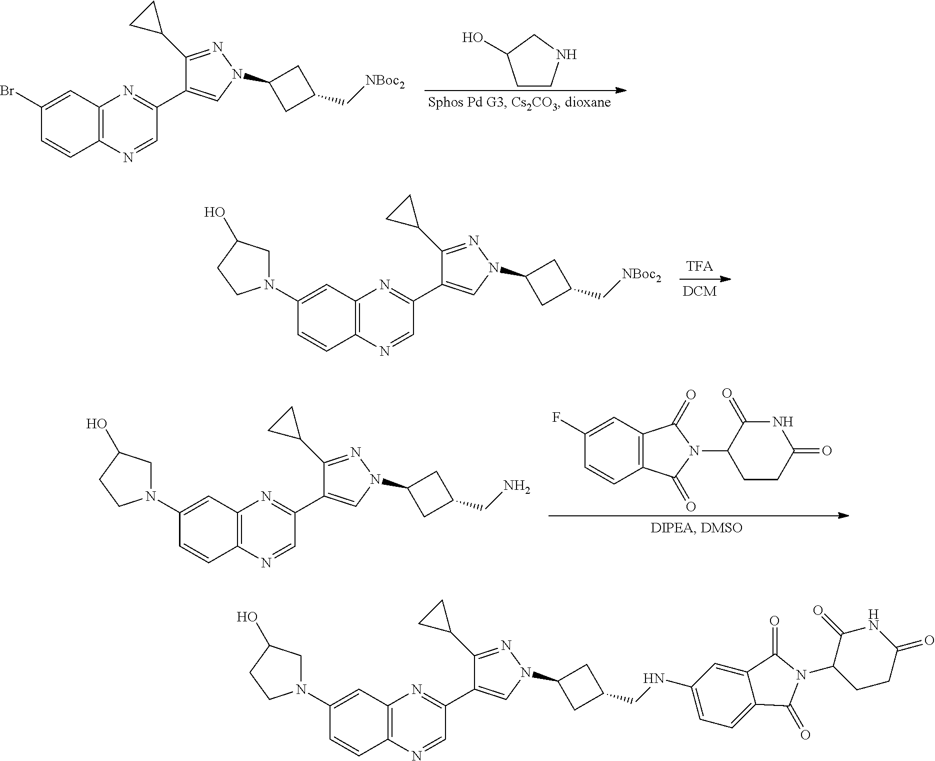

### Step 1. Synthesis of tert-butyl N-tert-butoxycarbonyl-N-((trans-3-(3-cyclopropyl-4-(7-(3-hydroxypyrrolidin-1-yl)quinoxalin-2-yl)pyrazol-1-yl)cyclobutyl)methyl)carbamate

To a solution of tert-butyl N-((trans-3-(4-(7-bromoquinoxalin-2-yl)-3-cyclopropyl-pyrazol-1-yl)cyclobutyl)methyl)-N-tert-butoxycarbonyl-carbamate (150 mg, 0.25 mmol) in dioxane (10 mL) were added pyrrolidin-3-ol (43.7 mg, 0.50 mmol), Sphos Pd G3 (43.4 mg, 0.05 mmol) and Cs₂CO₃ (163.4 mg, 0.50 mmol) at rt. The reaction mixture was stirred at 100° C. for 16 h under nitrogen. After cooling down to rt, the mixture was poured into water (30 mL) and extracted with ethyl acetate (3×20 mL). The combined organic layers were washed with saturated brine (20 mL), dried over anhydrous sodium sulfate, filtered and evaporated under reduced pressure. The resulting residue was purified by reverse-phase chromatography to provide the desired product (120 mg, 79% yield) as a red solid. MS (ESI) m/z=605.8 [M+H]⁺.

The remaining steps were performed according to the procedures for steps 3 to 4 of GS-641 to provide the desired product (1.5 mg, 10% yield over 2 steps) as a light-yellow solid. MS (ESI) m/z=661.6 [M+H]⁺.

### Example 21. 5-(((Trans-3-(3-cyclopropyl-4-(2-isopropyl-2H-pyrazolo[3,4-c]pyridin-7-yl)-1H-pyrazol-1-yl)cyclobutyl)methyl)amino)-2-(2,6-dioxopiperidin-3-yl)isoindoline-1,3-dione (GS-660)

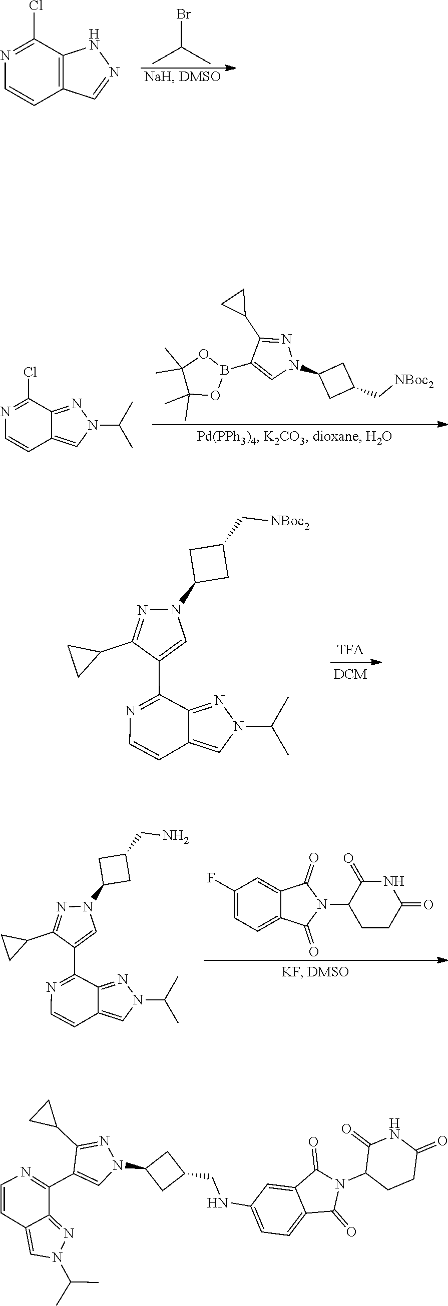

### Step 1. Synthesis of 7-chloro-2-isopropyl-2H-pyrazolo[3,4-c]pyridine

To a solution of 7-chloro-1H-pyrazolo[3,4-c]pyridine (600 mg, 3.91 mmol) in DMF (10 mL) was added NaH (312 mg, 7.81 mmol) in portions at 0 [symbol] under N₂. After the reaction was stirred at 0 [symbol] for 30 min, 2-bromopropane (961 mg, 7.81 mmol) in DMF (2 mL) was added dropwise at 0 [symbol] . The resulting mixture was stirred at 50 [symbol] for 2 h, before it was quenched with water (30 mL) slowly and extracted with ethyl acetate (3×20 mL). The combined organic phase was washed with brine, dried over anhydrous Na₂SO₄, filtered and concentrated. The residue was purified by silica gel chromatography to provide the desired isomer (420 mg, 54% yield) as a colorless oil. MS (ESI) m/z=196.2 [M+H]⁺.

### Step 2. Synthesis of tert-butyl N-tert-butoxycarbonyl-N-((trans-3-(3-cyclopropyl-4-(2-isopropylpyrazolo[3,4-c]pyridin-7-yl)pyrazol-1-yl)cyclobutyl)methyl)carbamate

To a solution of 7-chloro-2-isopropyl-pyrazolo[3,4-c]pyridine (100 mg, 0.5 mmol) and tert-butyl N-tert-butoxycarbonyl-N-((trans-3-(3-cyclopropyl-4-(4,4,5,5-tetramethyl-1,3,2-dioxaborolan-2-yl)pyrazol-1-yl)cyclobutyl)methyl)carbamate (528 mg, 1.02 mmol) in dioxane (10 mL) and H₂O (1 mL) were added Cs₂CO₃ (497 mg, 1.53 mmol) and Pd(PPh₃)₄ (59 mg, 0.05 mmol) at rt under N₂. The reaction mixture was stirred at 100° C. for 16 h. After cooling down to rt, the reaction mixture was diluted with water (10 mL) and extracted with ethyl acetate (3×20 mL). The combined organic layers were washed with brine, dried over anhydrous sodium sulfate, filtered and concentrated under reduced pressure. The residue was purified by reverse-phase chromatography to provide the desired product (32 mg, 11% yield) as a brown oil. MS (ESI) m/z=551.6 [M+H]⁺.

The remaining steps were performed according to the procedures for steps 2 to 3 of GS-642 to provide the desired product (1.3 mg, 3% yield over 2 steps) as a yellow solid. MS (ESI) m/z=607.8 [M+H]⁺.

### Example 22. 5-(((Trans-3-(3-cyclopropyl-4-(1-isopropyl-1H-pyrazolo[3,4-c]pyridin-7-yl)-1H-pyrazol-1-yl)cyclobutyl)methyl)amino)-2-(2,6-dioxopiperidin-3-yl)isoindoline-1,3-dion (GS-661)

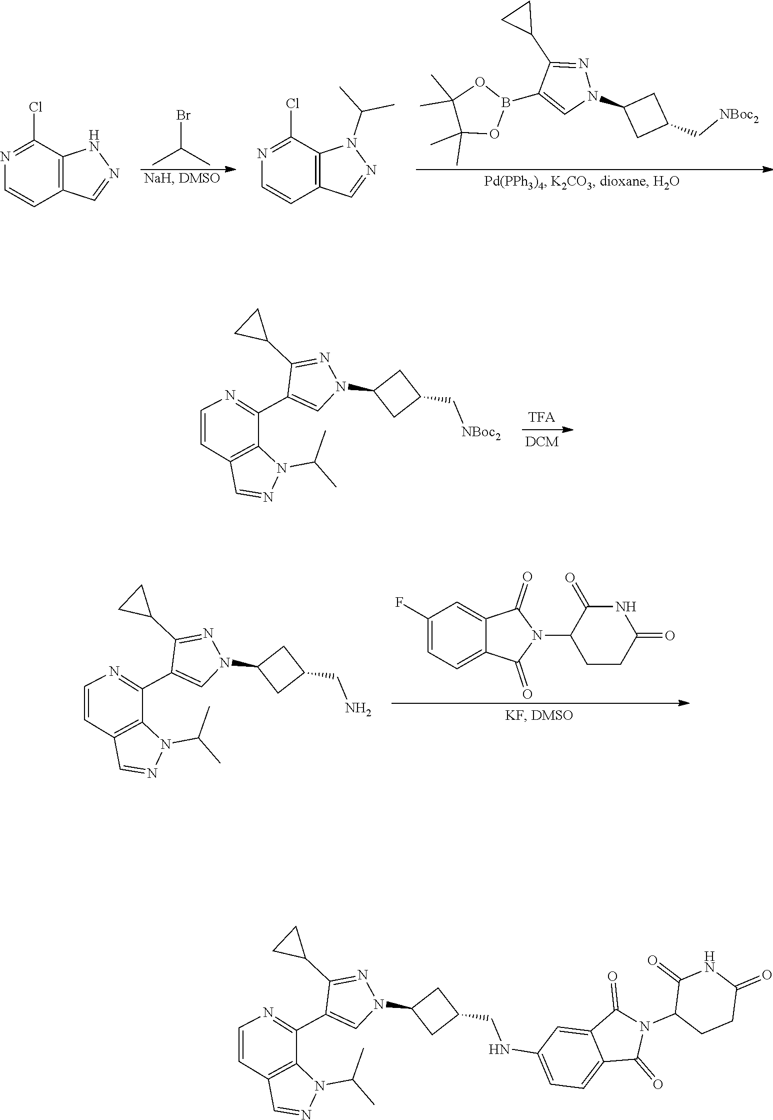

GS-661 was synthesized following the procedures for steps 1 to 2 of GS-660 and steps 2 to 3 of GS-642 (0.9 mg, 1% yield over 4 steps) as a yellow solid. MS (ESI) m/z=607.8 [M+H]⁺.

### Example 23. 3-(6-(((Trans-3-(3-cyclopropyl-4-(quinoxalin-2-yl)-1H-pyrazol-1-yl)cyclobutyl)methyl)amino)-1-oxoisoindolin-2-yl)piperidine-2,6-dione (GS-662)

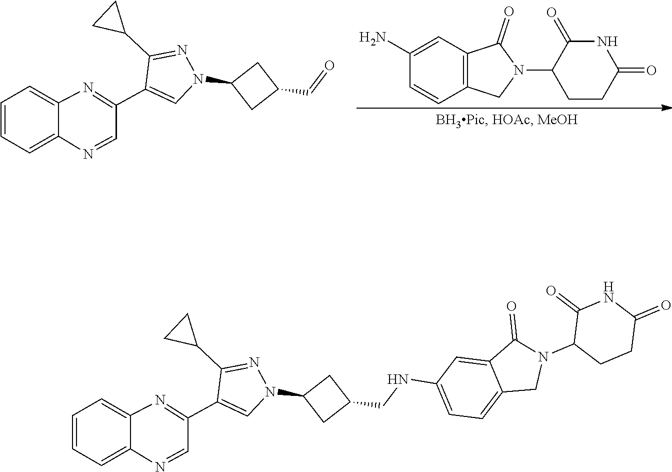 [SMILES: O=C[C@H]1C[C@H](n2cc(-c3cnc4ccccc4n3)c(C3CC3)n2)C1 ; Nc1ccc2c(c1)C(=O)N(C1CCC(=O)NC1=O)C2 ; O=C1CCC(N2Cc3ccc(NC[C@H]4C[C@H](n5cc(-c6cnc7ccccc7n6)c(C6CC6)n5)C4)cc3C2=O)C(=O)N1]

GS-662 was synthesized following the procedure for step 2 of GS-651 (17.9 mg, 25% yield) as an off-white solid. MS (ESI) m/z=562.5 [M+H]⁺.

### Example 24. 5-(((Trans-3-(3-cyclopropyl-1H-pyrazolo[3,4-d]pyrimidin-1-yl)cyclobutyl)methyl)amino)-2-(2,6-dioxopiperidin-3-yl)isoindoline-1,3-dione (GS-663)

### Step 1. Synthesis of cyclopropyl(4,6-dichloropyrimidin-5-yl)methanol

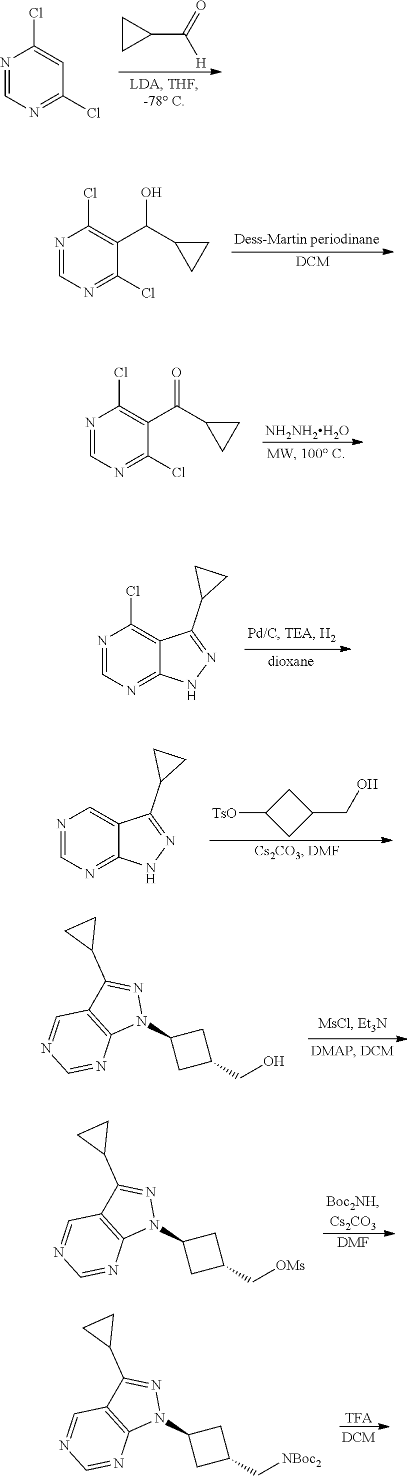

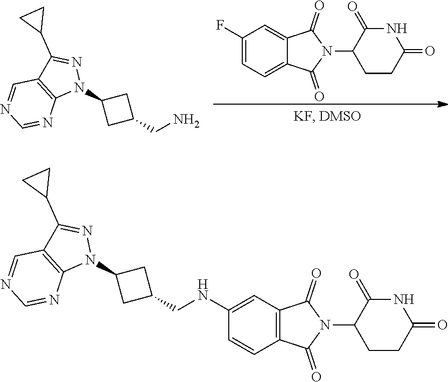 [SMILES: NC[C@H]1C[C@H](n2nc(C3CC3)c3cncnc32)C1 ; O=C1CCC(N2C(=O)c3ccc(F)cc3C2=O)C(=O)N1 ; O=C1CCC(N2C(=O)c3ccc(NC[C@H]4C[C@H](n5nc(C6CC6)c6cncnc65)C4)cc3C2=O)C(=O)N1]

To a solution of diisopropylamine (3.75 g, 37.2 mmol) in THF (50 mL) was added n-BuLi (2.5 M, 15 mL, 37.2 mmol) dropwise at −78° C. under nitrogen. The reaction mixture was stirred at the same temperature for 30 min, then a solution of 4,6-dichloropyrimidine (5.0 g, 33.8 mmol) in THF (15 mL) was added dropwise. The reaction mixture was stirred at −78° C. for an additional 30 min, before cyclopropane carbaldehyde (2.37 g, 33.8 mmol) was added dropwise. After the reaction mixture was warmed up to rt, it was quenched with water (50 mL), and extracted with ethyl acetate (50 mL×3). The combined organic layers were washed with brine, dried over anhydrous sodium sulfate, filtered and concentrated under reduced pressure. The residue was purified by silica gel column chromatography (petroleum ether/ethyl acetate=5:1) to provide the desired product (4.25 g, 58% yield) as a colorless oil. MS (ESI) m/z=219.0 [M+H]⁺.

### Step 2. Synthesis of cyclopropyl(4,6-dichloropyrimidin-5-yl)methanone

To a solution of cyclopropyl(2,4-dichloropyridin-3-yl)methanol (4.25 g, 19.59 mmol) in dichloromethane (50 mL) was added Dess-Martin periodinane (8.3 g, 19.59 mmol). The reaction mixture was stirred at rt overnight before it was quenched with saturated sodium bicarbonate aqueous solution (50 mL) and 10% sodium sulfite aqueous solution (50 mL). After stirring for 15 min, the reaction mixture was extracted with dichloromethane (50 mL×4). The combined organic layers were washed with brine, dried over anhydrous sodium sulfate, filtered and concentrated under reduced pressure. The residue was purified by silica gel column chromatography (petroleum ether/ethyl acetate=5:1) to provide the desired product (2.9 g, 69% yield) as a colorless oil. MS (ESI) m/z=217.0 [M+H]⁺.

### Step 3. Synthesis of 4-chloro-3-cyclopropyl-1H-pyrazolo[3,4-d]pyrimidine

A solution of cyclopropyl(4,6-dichloropyrimidin-5-yl)methanone (2.9 g, 13.4 mmol) in hydrazine hydrate (20 mL) was heated at 100° C. for 1.5 h under microwave irradiation. After cooling down to rt, the reaction mixture was poured into water and extracted with dichloromethane (20 mL×4). The combined organic layers were washed with brine, dried over anhydrous sodium sulfate, filtered and concentrated under reduced pressure. The residue was purified by silica gel column chromatography (petroleum ether/ethyl acetate=5:1) to provide the desired product (2.4 g, 92% yield) as a white solid. ¹H NMR (400 MHz, DMSO-d₆) δ 14.01 (s, 1H), 8.75 (s, 1H), 2.51-2.46 (m, 1H), 1.09-0.94 (m, 4H). MS (ESI) m/z=195.1 [M+H]⁺.

### Step 4. Synthesis of 3-cyclopropyl-1H-pyrazolo[3,4-d]pyrimidine

To a solution of 4-chloro-3-cyclopropyl-1H-pyrazolo[3,4-d]pyrimidine (1.4 g, 7.2 mmol) in 1,4-dioxane (30 mL) were added triethylamine (729 mg, 7.2 mmol) and Pd/C (300 mg). The reaction mixture was stirred at rt overnight under H₂ atmosphere. The mixture was filtered through Celite and the filtrate was concentrated under reduced pressure. The residue was purified by silica gel column chromatography (petroleum ether/ethyl acetate=5:1) to provide the desired product (600 mg, 52% yield) as a white solid. ¹H NMR (400 MHz, DMSO-d₆) δ 13.58 (s, 1H), 9.31 (s, 1H), 8.91 (s, 1H), 2.42-2.35 (m, 1H), 1.10-1.02 (m, 4H). MS (ESI) m/z=161.1 [M+H]⁺.

The remaining steps were performed according to the procedures for step 4 of GS-648 and steps 5 to 8 of GS-643 to provide the desired product (1.5 mg, 1% yield over 5 steps) as a yellow solid. MS (ESI) m/z=500.5 [M+H]⁺.

### Example 25. 5-(((Trans-3-(3-cyclopropyl-4-(1-isopropyl-1H-imidazo[4,5-c]pyridin-6-yl)-1H-pyrazol-1-yl)cyclobutyl)methyl)amino)-2-(2,6-dioxopiperidin-3-yl)isoindoline-1,3-dione (GS-664)

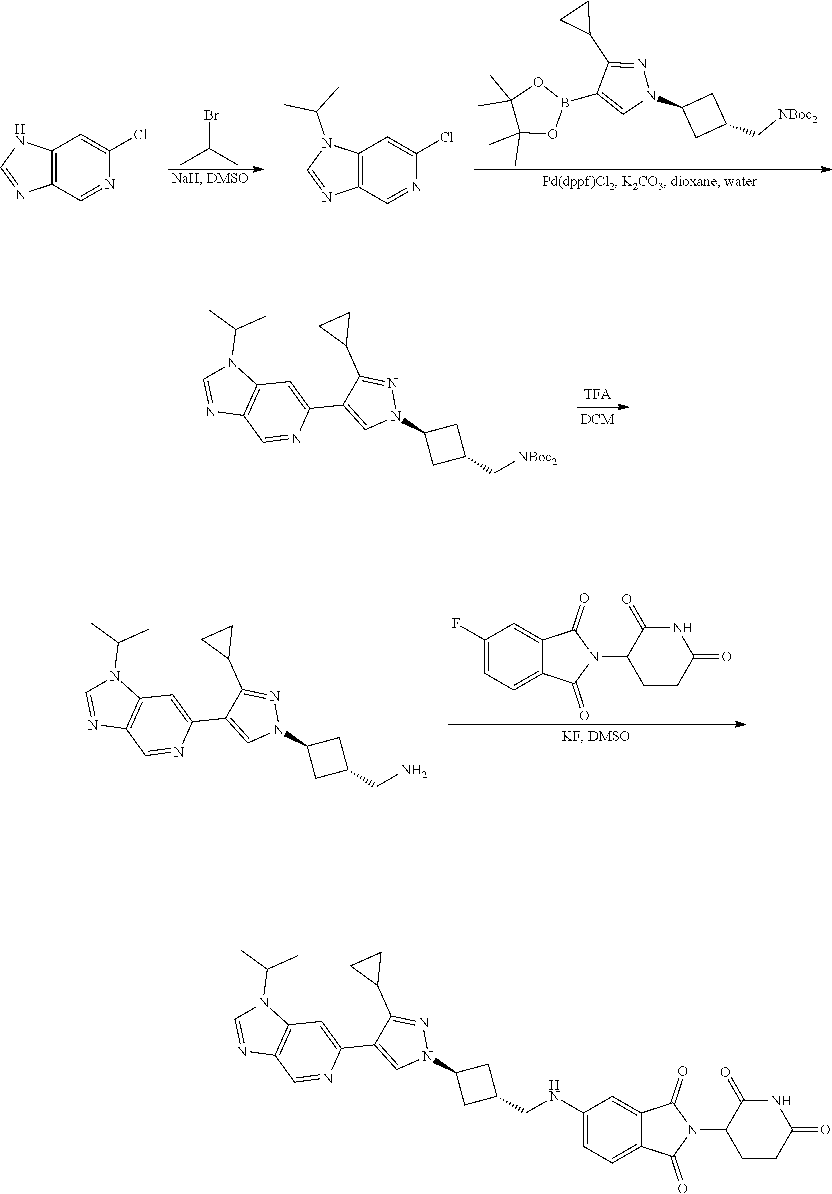

GS-664 was synthesized following the standard procedures for steps 1 to 2 of GS-660 and steps 2 to 3 of GS-642 (3.1 mg, 2% yield over 4 steps) as a light-yellow solid. MS (ESI) m/z=607.5 [M+H]⁺.

### Example 26. 5-(((Trans-3-(3-cyclopropyl-4-(3-(piperidin-4-yl)pyridin-2-yl)-1H-pyrazol-1-yl)cyclobutyl)methyl)amino)-2-(2,6-dioxopiperidin-3-yl)isoindoline-1,3-dione (GS-665)

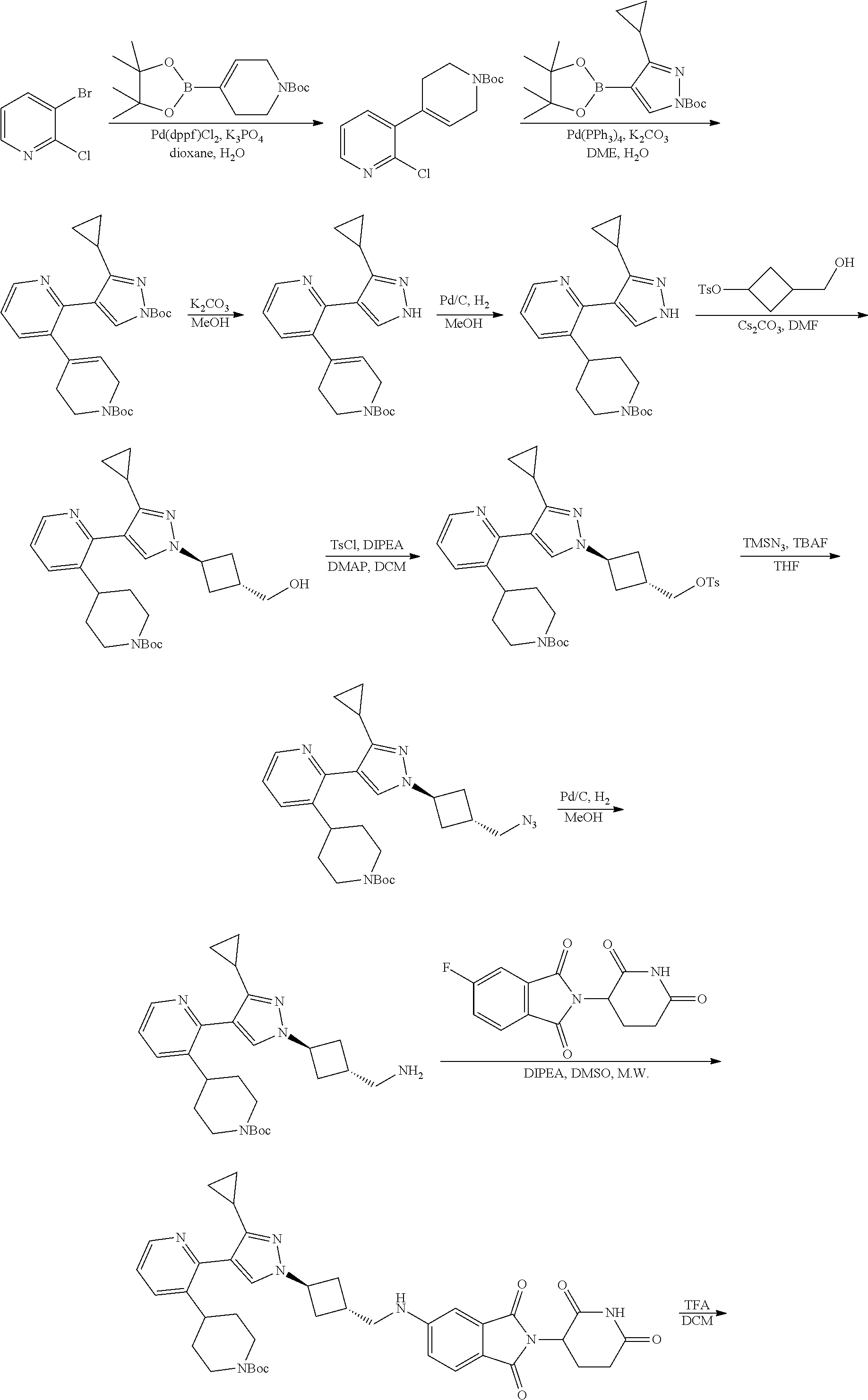

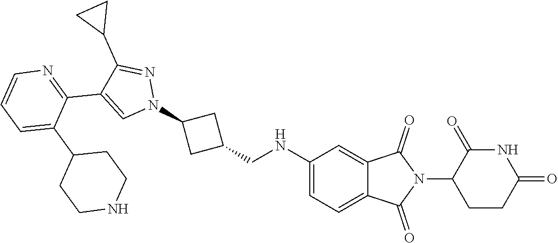 [SMILES: O=C1CCC(N2C(=O)c3ccc(NC[C@H]4C[C@H](n5cc(-c6ncccc6C6CCNCC6)c(C6CC6)n5)C4)cc3C2=O)C(=O)N1]

### Step 1. Synthesis of tert-butyl 2-chloro-3′,6′-dihydro-[3,4′-bipyridine]-1′(2′H)-carboxylate

To a solution of 3-bromo-2-chloropyridine (2 g, 10.39 mmol), tert-butyl 4-(4,4,5,5-tetramethyl-1,3,2-dioxaborolan-2-yl)-3,6-dihydropyridine-1 (2H)-carboxylate (4.18 g, 13.51 mmol) and K₃PO₄ (4.41 g, 20.79 mmol) in dioxane (15 mL) and H₂O (2 mL) was added Pd(dppf)Cl₂ (760.4 mg, 1.04 mmol). The reaction mixture was stirred at 90 [symbol] for 12 h. After cooling down to rt, the mixture was quenched with H₂O (20 mL) and extracted with ethyl acetate (20 mL×3). The combined organic phase was washed with brine, dried over anhydrous Na₂SO₄, filtered and concentrated under reduced pressure. The residue was purified by silica gel chromatography to provide the desired product (2.4 g, 78% yield) as a yellow oil. MS (ESI) m/z=295.3 [M+H]⁺.

### Step 2. Synthesis of tert-butyl 2-(1-(tert-butoxycarbonyl)-3-cyclopropyl-1H-pyrazol-4-yl)-3′,6′-dihydro-[3,4′-bipyridine]-1′(2′H)-carboxylate

To a solution of tert-butyl 2-chloro-3′,6′-dihydro-[3,4′-bipyridine]-1′(2′H)-carboxylate (1 g, 3.39 mmol), tert-butyl 3-cyclopropyl-4-(4,4,5,5-tetramethyl-1,3,2-dioxaborolan-2-yl)-1H-pyrazole-1-carboxylate (1.7 g, 5.09 mmol) and K₂CO₃ (1.41 g, 10.18 mmol) in DME (20 mL) and H₂O (2 mL) was added Pd(PPh₃)₄ (392.0 mg, 0.34 mmol) at rt. The reaction mixture was stirred at 90 [symbol] for 12 h. After cooling down to it, the mixture was quenched with H₂O (20 mL) and extracted with ethyl acetate (20 mL×3). The combined organic phase was washed with brine, dried over anhydrous Na₂SO₄, filtered and concentrated under reduced pressure. The residue was purified by silica gel chromatography to provide the desired product (1 g, 63% yield) as a yellow oil. MS (ESI) m/z=467.8 [M+H]⁺.

### Step 3. Synthesis of tert-butyl 2-(3-cyclopropyl-1H-pyrazol-4-yl)-3′,6′-dihydro-[3,4′-bipyridine]-1′(2′H)-carboxylate

To a solution of tert-butyl 2-(1-(tert-butoxycarbonyl)-3-cyclopropyl-1H-pyrazol-4-yl)-3′,6′-dihydro-[3,4′-bipyridine]-1′(2′H)-carboxylate (1 g, 2.14 mmol) in methanol (5 mL) was added K₂CO₃ (888.6 mg, 6.43 mmol). After the reaction mixture was stirred at 25 [symbol] for 1 h, it was quenched with H₂O (20 mL) and extracted with dichloromethane (20 mL×3). The combined organic phase was washed with brine, dried over anhydrous Na₂SO₄, filtered and concentrated under reduced pressure. The residue was purified by silica gel chromatography to provide the desired product (350 mg, 44% yield) as a yellow solid. MS (ESI) m/z=367.5 [M+H]⁺.

### Step 4. Synthesis of tert-butyl 4-(2-(3-cyclopropyl-1H-pyrazol-4-yl)pyridin-3-yl)piperidine-1-carboxylate

To a solution tert-butyl 2-(3-cyclopropyl-1H-pyrazol-4-yl)-3′,6′-dihydro-[3,4′-bipyridine]-1′(2′H)-carboxylate (330 mg, 0.9 mmol) in methanol (10 mL) was added 10% Pd/C (30 mg). The reaction mixture was purged three times with hydrogen and stirred at 40° C. for 12 h under hydrogen atmosphere. The mixture was filtered through Celite and the filtrate was concentrated under reduced pressure to provide the desired product (300 mg, 90% yield) as a yellow oil. This compound was used directly in the next step without further purification. MS (ESI) m/z=369.6 [M+H]⁺.

### Step 5. Synthesis of tert-butyl 4-(2-(3-cyclopropyl-1-(trans-3-(hydroxymethyl)cyclobutyl)-1H-pyrazol-4-yl)pyridin-3-yl)piperidine-1-carboxylate

To a solution of tert-butyl 4-(2-(3-cyclopropyl-1H-pyrazol-4-yl)pyridin-3-yl)piperidine-1-carboxylate (280 mg, 0.76 mmol) and 3-(hydroxymethyl)cyclobutyl 4-methylbenzenesulfonate (292.16 mg, 1.14 mmol) in DMF (5 mL) was added Cs₂CO₃ (495.2 mg, 1.52 mmol) at rt. The reaction mixture was stirred at 90 [symbol] for 12 h. After cooling down to rt, the mixture was quenched with H₂O (20 mL) and extracted with ethyl acetate (20 mL×3). The combined organic phase was washed with brine, dried over anhydrous Na₂SO₄, filtered and concentrated under reduced pressure. The residue was purified by silica gel chromatography to provide the desired trans-isomer (200 mg, 58% yield) as a yellow solid. MS (ESI) m/z=453.6 [M+H]⁺.

### Step 6. Synthesis of tert-butyl 4-(2-(3-cyclopropyl-1-(trans-3-((tosyloxy)methyl)cyclobutyl)-1H-pyrazol-4-yl)pyridin-3-yl)piperidine-1-carboxylate

To a solution of tert-butyl 4-(2-(3-cyclopropyl-1-(trans-3-(hydroxymethyl)cyclobutyl)-1H-pyrazol-4-yl)pyridin-3-yl)piperidine-1-carboxylate (200 mg, 0.44 mmol), DMAP (54.0 mg, 0.44 mmol) and DIPEA (0.2 mL) in dichloromethane (10 mL) was added tosyl chloride (168.5 mg, 0.88 mmol) at rt. After the reaction mixture was stirred at it for 12 h, it was quenched with H₂O (20 mL) and extracted with dichloromethane (20 mL×3). The combined organic phase was washed with brine, dried over anhydrous Na₂SO₄, filtered and concentrated under reduced pressure. The residue was purified by silica gel chromatography to provide the desired product (140 mg, 52% yield) as a yellow oil. MS (ESI) m/z=607.6 [M+H]⁺.

### Step 7. Synthesis of tert-butyl 4-(2-(1-(trans-3-(azidomethyl)cyclobutyl)-3-cyclopropyl-1H-pyrazol-4-yl)pyridin-3-yl)piperidine-1-carboxylate

To a solution of tert-butyl 4-(2-(3-cyclopropyl-1-(trans-3-(hydroxymethyl)cyclobutyl)-1H-pyrazol-4-yl)pyridin-3-yl)piperidine-1-carboxylate (130 mg, 0.21 mmol) and TMSN₃ (74.1 mg, 0.63 mmol) in THF (10 mL) was added TBAF (1 M, 0.6 mL) at rt. The reaction mixture was stirred at 60 [symbol] for 1 h. After cooling down to rt, the mixture was quenched with H₂O (20 mL) and extracted with dichloromethane (20 mL×3). The combined organic phase was washed with brine, dried over anhydrous Na₂SO₄, filtered and concentrated under reduced pressure. The residue was purified by silica gel chromatography to provide the desired product (80 mg, 78% yield) as a yellow oil. MS (ESI) m/z=478.7 [M+H]⁺.

### Step 8. Synthesis of tert-butyl 4-(2-(1-(trans-3-(aminomethyl)cyclobutyl)-3-cyclopropyl-1H-pyrazol-4-yl)pyridin-3-yl)piperidine-1-carboxylate

To a solution of tert-butyl 4-(2-(1-(trans-3-(azidomethyl)cyclobutyl)-3-cyclopropyl-1H-pyrazol-4-yl)pyridin-3-yl)piperidine-1-carboxylate (40 mg, 0.084 mmol) in methanol (2 mL) was added 10% Pd/C (10 mg). The reaction mixture was purged three times with hydrogen and stirred at rt for 12 h under hydrogen atmosphere. The mixture was filtered through Celite and the filtrate was concentrated under reduced pressure to provide the desired product (35 mg, 92% yield) as a yellow oil. This compound was used directly in the next step without further purification. MS (ESI) m/z=452.5 [M+H]⁺.

### Step 9. Synthesis of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-3-yl)piperidine-1-carboxylate

A mixture of tert-butyl 4-(2-(1-(trans-3-(aminomethyl)cyclobutyl)-3-cyclopropyl-1H-pyrazol-4-yl)pyridin-3-yl)piperidine-1-carboxylate (35 mg, 0.078 mmol), 2-(2,6-dioxo-3-piperidyl)-5-fluoro-isoindoline-1,3-dione (25.7 mg, 0.093 mmol) and DIPEA (0.1 mL) in DMSO (1 mL) was stirred at 130 [symbol] for 0.5 h under microwave irradiation. After cooling down to rt, the mixture was purified by reverse-phase chromatography to provide the desired product (20 mg, 36% yield) as a yellow solid. MS (ESI) m/z=708.6 [M+H]⁺.

### Step 10. Synthesis of 5-(((trans-3-(3-cyclopropyl-4-(3-(piperidin-4-yl)pyridin-2-yl)-1H-pyrazol-1-yl)cyclobutyl)methyl)amino)-2-(2,6-dioxopiperidin-3-yl)isoindoline-1,3-dione

To a solution of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-3-yl)piperidine-1-carboxylate (20 mg, 0.028 mmol) in dichloromethane (5 mL) was added TFA (1 mL). After the reaction mixture was stirred at rt for 1 h, it was concentrated under reduced pressure. The residue was purified by reverse-phase chromatography to provide the desired product (2.1 mg, 11% yield) as a yellow solid. MS (ESI) m/z=608.6 [M+H]⁺.

### Example 27. 5-(((Trans-3-(3-cyclopropyl-4-((1-methylpiperidin-4-yl)amino)-1H-pyrazol-1-yl)cyclobutyl)methyl)amino)-2-(2,6-dioxopiperidin-3-yl)isoindoline-1,3-dione (GS-666)

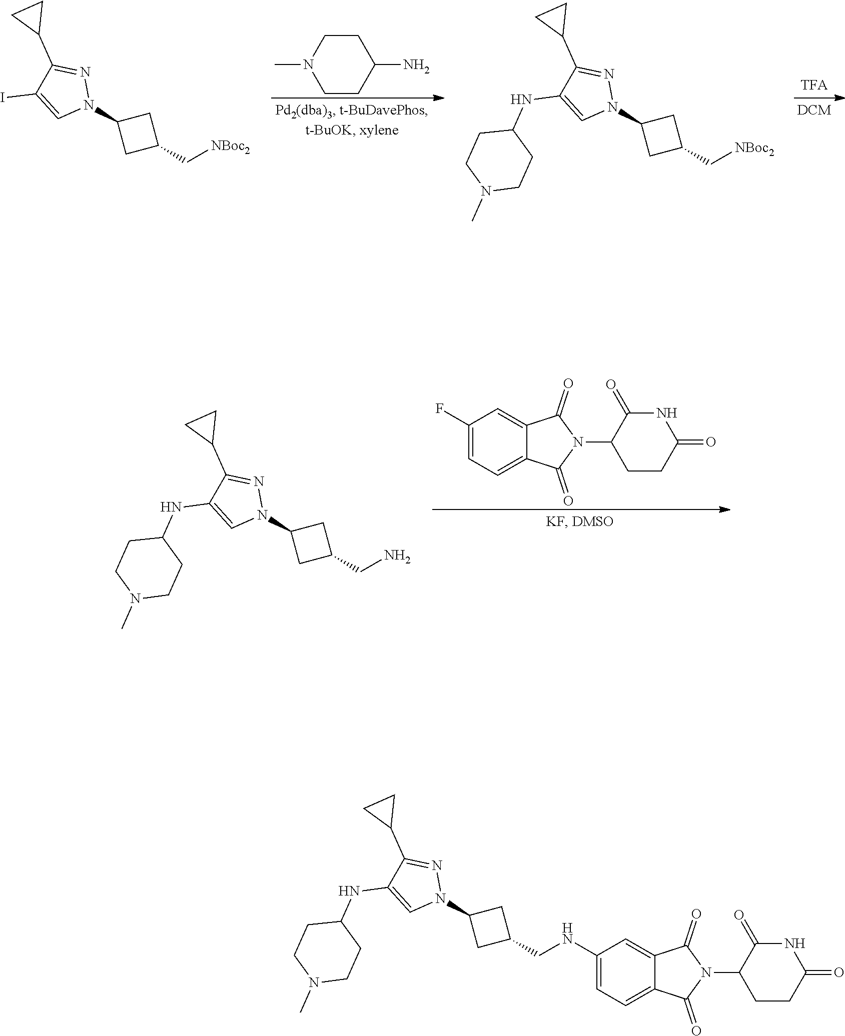

### Step 1. Synthesis of tert-butyl N-((trans-3-(3-cyclopropyl-4-((1-methyl-4-piperidyl)amino)pyrazol-1-yl)cyclobutyl)methyl)carbamate

To a mixture of tert-butyl-N-tert-butoxycarbonyl-N-((trans-3-(3-cyclopropyl-4-iodo-pyrazol-1-yl)cyclobutyl)methyl)carbamate (150 mg, 0.29 mmol), 1-methylpiperidin-4-amine (99.3 mg, 0.87 mmol), t-BuOK (80.5 mg, 0.72 mmol) and t-BuDavephos (122.3 mg, 0.29 mmol) in m-xylene (8 mL) was added Pd₂(dba)₃ (19.2 mg, 0.021 mmol) at rt. The reaction mixture was purged with N₂ and stirred at 100 [symbol] for 1 h. After cooling down to rt, the reaction mixture was diluted with EtOAc (20 mL) and filtered through Celite. The filtrate was concentrated and purified by reverse-phase chromatography to provide the desired product (20 mg, 17% yield) as a yellow solid. MS (ESI) m/z=404.5 [M+H]⁺.

The remaining steps were performed according to the procedures for steps 2 to 3 of GS-642 to provide the desired product (3.8 mg, 9% yield over 2 steps) as a yellow solid. MS (ESI) m/z=560.7 [M+H]⁺.

### Example 28. 5-(((Trans-3-(4-(cyclohexylamino)-3-cyclopropyl-1H-pyrazol-1-yl)cyclobutyl)methyl)amino)-2-(2,6-dioxopiperidin-3-yl)isoindoline-1,3-dione (GS-667)

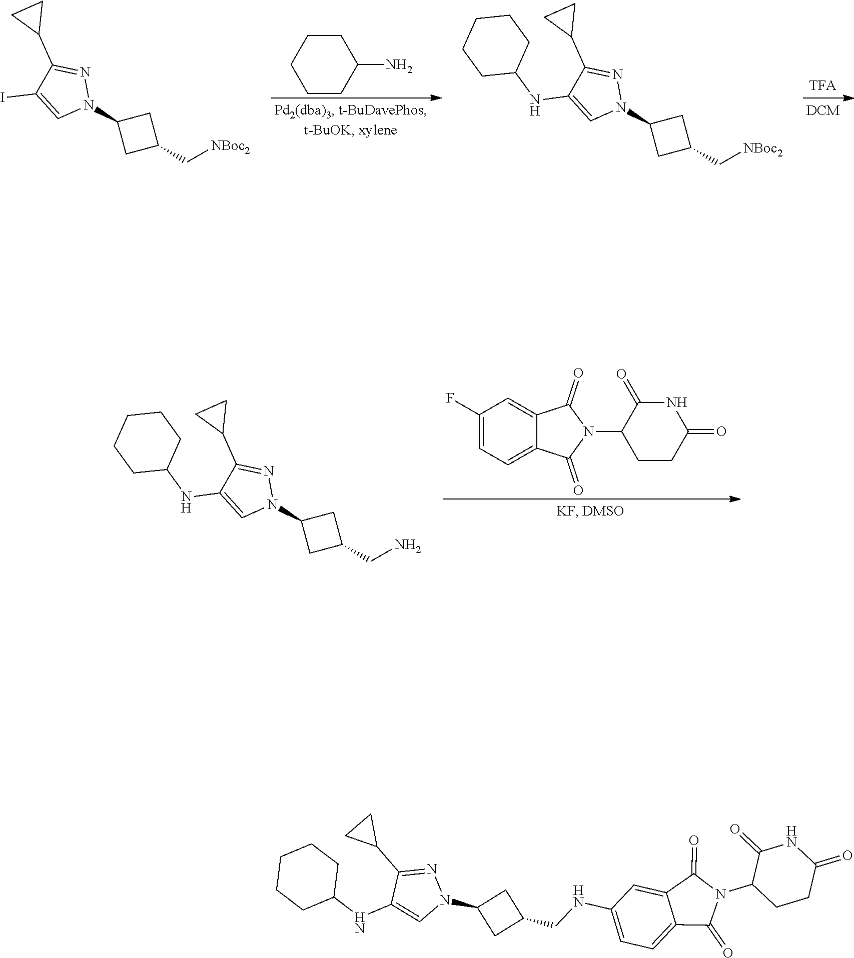

GS-667 was synthesized following the standard procedures for step 1 of GS-666 and steps 2 to 3 of GS-642 (2.4 mg, 4% yield over 3 steps) as a yellow solid. MS (ESI) m/z=545.7 [M+H]⁺.

### Example 29. 3-(5-(((Trans-3-(3-(difluoromethyl)-4-(5-fluoro-3-methylpyridin-2-yl)-1H-pyrazol-1-yl)cyclobutyl)methyl)amino)-1-oxoisoindolin-2-yl)piperidine-2,6-dione (GS-668)

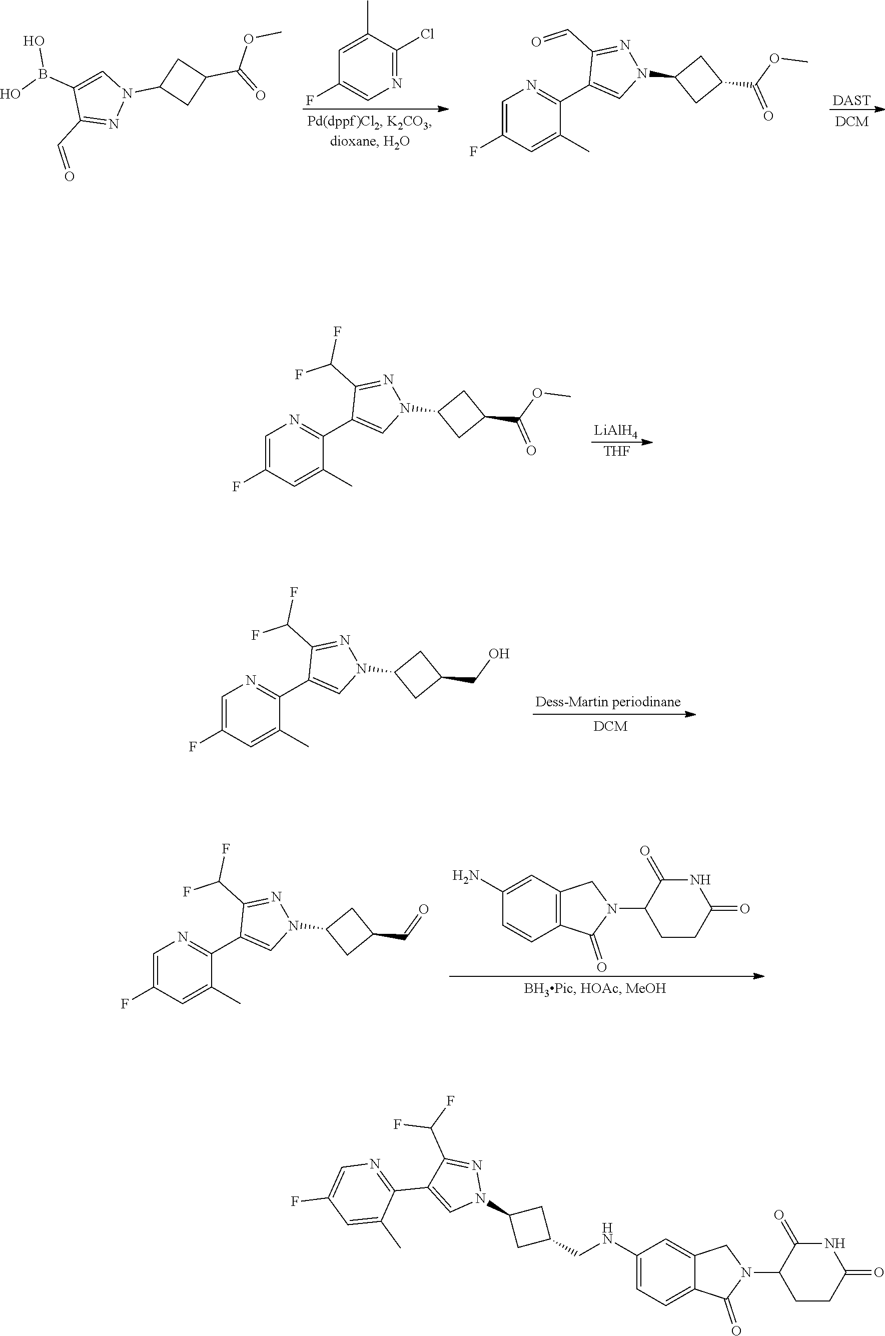

GS-668 was synthesized following the standard procedures for steps 1 to 5 of GS-677 (30 mg, 6% yield over 5 steps) as a yellow solid. MS (ESI) m/z=553.5 [M+H]⁺.

### Example 30. 5-(((Trans-3-(3-cyclopropyl-4-(pyridin-2-ylamino)-1H-pyrazol-1-yl)cyclobutyl)methyl)amino)-2-(2,6-dioxopiperidin-3-yl)isoindoline-1,3-dione (GS-669)

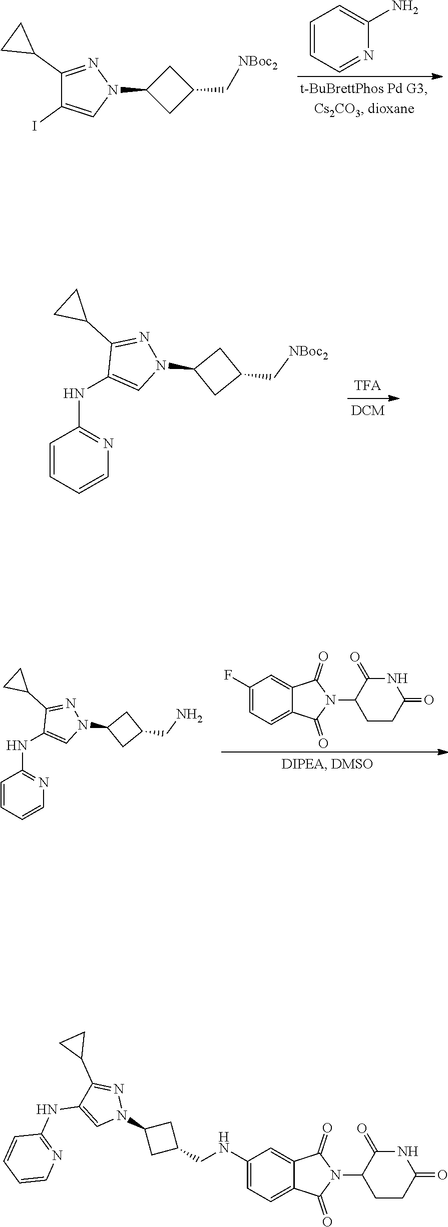

### Step 1. Synthesis of tert-butyl N-tert-butoxycarbonyl-N-((trans-3-(3-cyclopropyl-4-(2-pyridylamino)pyrazol-1-yl)cyclobutyl)methyl)carbamate

To a solution of pyridin-2-amine (60.0 mg, 0.64 mmol) in dioxane (5 mL) were added tert-butyl N-tert-butoxycarbonyl-N-((trans-3-(3-cyclopropyl-4-iodo-pyrazol-1-yl)cyclobutyl)methyl)carbamate (329.8 mg, 0.64 mmol), t-BuBrettPhos Pd G3 (54.5 mg, 0.064 mmol) and Cs₂CO₃ (415.7 mg, 1.28 mmol) at rt. The reaction mixture was stirred at 100 [symbol] for 12 h under nitrogen. After cooling down to rt, the reaction mixture was poured into water (10 mL) and extracted with ethyl acetate (3×10 mL). The combined organic layers were washed with saturated brine (20 mL), dried over anhydrous sodium sulfate, filtered and evaporated under reduced pressure. The resulting residue was purified by reverse-phase chromatography to provide the desired product (60.0 mg, 19% yield) as a pale-white solid. MS (ESI) m/z=484.6 [M+H]⁺.

The remaining steps were performed according to the procedures for steps 3 to 4 of GS-641 to provide the desired product (4.1 mg, 2% yield over 2 steps) as a yellow solid. MS (ESI) m/z=540.5 [M+H]⁺.

### Example 31. 5-(((Trans-3-(3-cyclopropyl-4-(pyridin-3-ylamino)-1H-pyrazol-1-yl)cyclobutyl)methyl)amino)-2-(2,6-dioxopiperidin-3-yl)isoindoline-1,3-dione (GS-670)

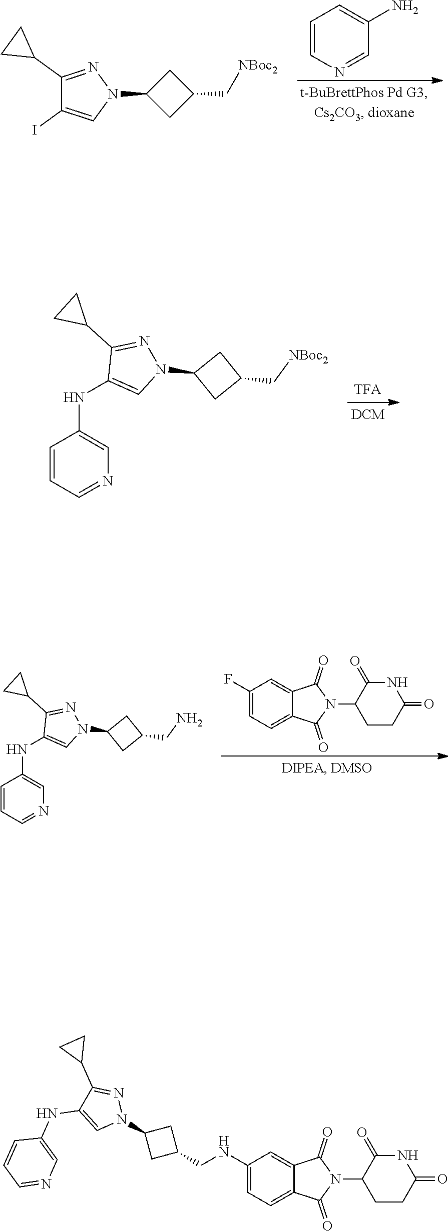

GS-670 was synthesized following the standard procedures for step 1 of GS-669 and steps 3 to 4 of GS-641 (9.6 mg, 1% yield over 3 steps) as a yellow solid. MS (ESI) m/z=540.5 [M+H]⁺.

### Example 32. 5-(((Trans-3-(3-cyclopropyl-4-(pyridin-4-ylamino)-1H-pyrazol-1-yl)cyclobutyl)methyl)amino)-2-(2,6-dioxopiperidin-3-y)isoindoline-1,3-dione (GS-671)

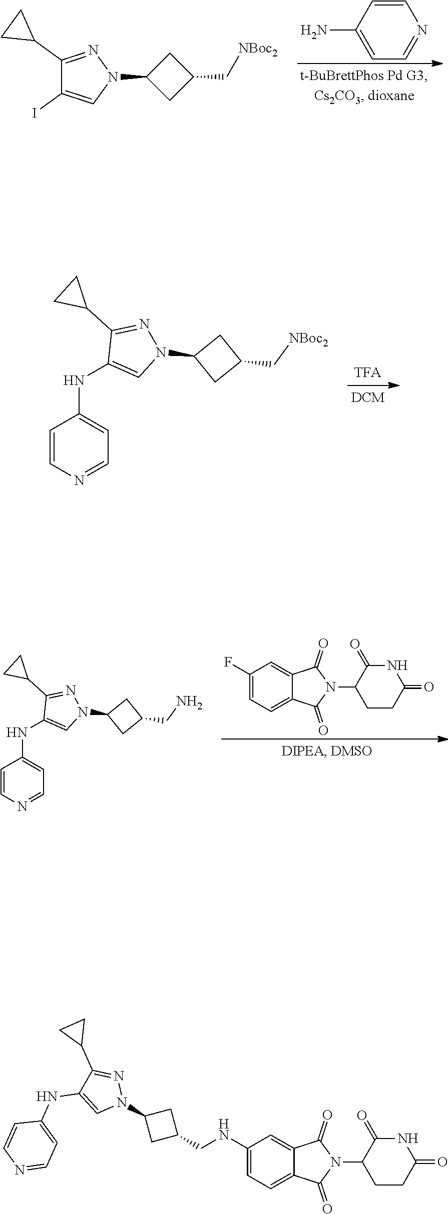

GS-671 was synthesized following the standard procedures for step 1 of GS-669 and steps 3 to 4 of GS-641 (10 mg, 17% yield over 3 steps) as a yellow solid. MS (ESI) m/z=540.5 [M+H]⁺.

### Example 33. 5-(((Trans-3-(3-cyclopropyl-6-methyl-1H-pyrazolo[3,4-b]pyridin-1-yl)cyclobutyl)methyl)amino)-2-(2,6-dioxopiperidin-3-yl)isoindoline-1,3-dione (GS-672)

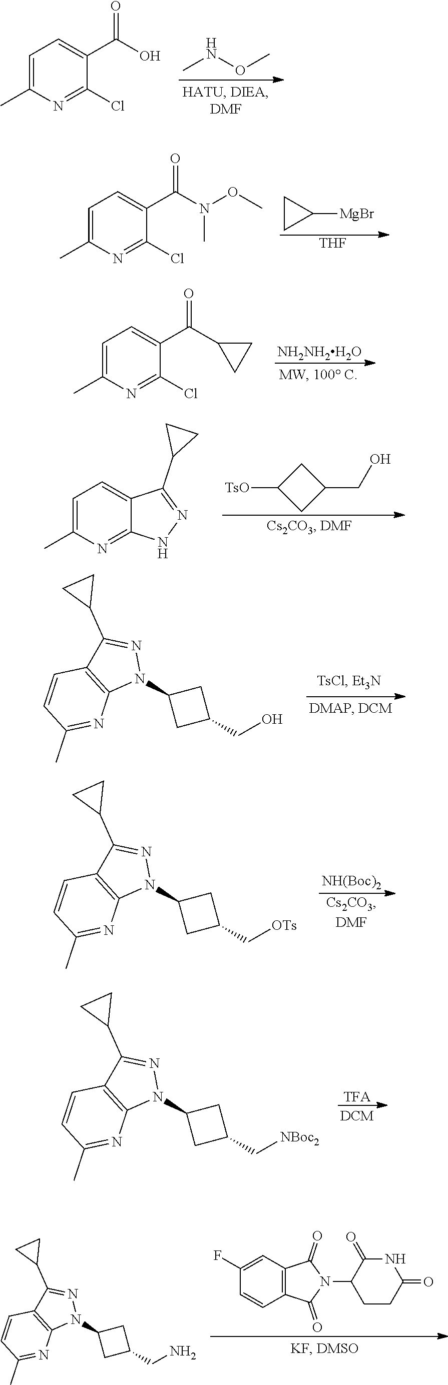

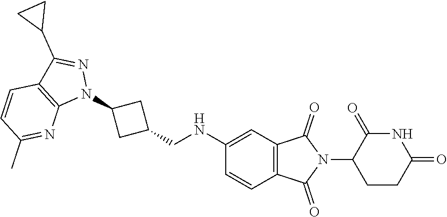 [SMILES: Cc1ccc2c(C3CC3)nn([C@H]3C[C@H](CNc4ccc5c(c4)C(=O)N(C4CCC(=O)NC4=O)C5=O)C3)c2n1]

### Step 1. Synthesis of 2-chloro-N-methoxy-N,6-dimethylnicotinamide

To a solution of 2-chloro-6-methylnicotinic acid (2.0 g, 11.7 mmol) in DMF (20 mL) were added HATU (6.7 g, 17.6 mmol), DIEA (4.5 g, 35.1 mmol) and N,O-dimethylhydroxylamine (1.3 g, 12.9 mmol) at rt. The reaction mixture was stirred at rt overnight. The reaction was quenched with water (100 mL) and extracted with ethyl acetate (3×50 mL). The combined organic layers were washed with saturated brine (50 mL), dried over anhydrous sodium sulfate, filtered and evaporated under reduced pressure. The residue was purified by column chromatography on silica-gel (petroleum ether/ethyl acetate=2:1) to provide the desired product (2.14 g, 86% yield) as a yellow solid. MS (ESI) m/z=215.1 [M+H]⁺.

### Step 2. Synthesis of (2-chloro-6-methylpyridin-3-yl)(cyclopropyl)methanone

To a solution of 2-chloro-N-methoxy-N,6-dimethylnicotinamide (2 g, 9.3 mmol) in THE (20 mL) was added a solution of cyclopropylmagnesium bromide (1.0 M in THF, 47 mL, 47 mmol) dropwise at 0 [symbol] . The reaction mixture was stirred at rt for 4 h, before it was quenched with aq. NH₄Cl solution (50 mL) slowly and extracted with ethyl acetate (3×50 mL). The combined organic layers were washed with saturated brine (50 mL), dried over anhydrous sodium sulfate, filtered and evaporated under reduced pressure. The residue was purified by column chromatography on silica-gel (petroleum ether/ethyl acetate=5:1) to provide the desired product (1.68 g, 93% yield) as a yellow oil. MS (ESI) m/z=196.0 [M+H]⁺.

### Step 3. Synthesis of 3-cyclopropyl-6-methyl-1H-pyrazolo[3,4-b]pyridine

A solution of (3-chloropyrazin-2-yl)(cyclopropyl)methanone (2.3 g, 12 mmol) in hydrazine hydrate (5 mL) was heated at 100° C. for 1.5 h under microwave irradiation. After cooling down to rt, the reaction mixture was poured into water and extracted with dichloromethane (20 mL×4). The combined organic layers were washed with saturated brine (20 mL), dried over anhydrous sodium sulfate, filtered and evaporated under reduced pressure. The residue was purified by column chromatography on silica-gel (petroleum ether/ethyl acetate=5:1) to provide the desired product (1.5 g, 70% yield) as a white solid. ¹H NMR (400 MHz, DMSO-d₆) δ 12.89 (s, 1H), 8.06 (d, J=8.4 Hz, 1H), 6.99 (d, J=8 Hz, 1H), 2.54 (s, 3H), 2.23-2.21 (m, 1H), 1.01-0.92 (m, 4H). MS (ESI) m/z=174.2 [M+H]⁺.

The remaining steps were performed according to the procedures for step 4 of GS-648 and steps 5 to 8 of GS-643 to provide the desired product (7.4 mg, 2% yield over 5 steps) as a yellow solid. MS (ESI) m/z=513.5 [M+H]⁺.

### Example 34. 5-(((Trans-3-(3-cyclopropyl-5-methyl-1H-pyrazolo[3,4-b]pyridin-1-yl)cyclobutyl)methyl)amino)-2-(2,6-dioxopiperidin-3-yl)isoindoline-1,3-dione (GS-673)

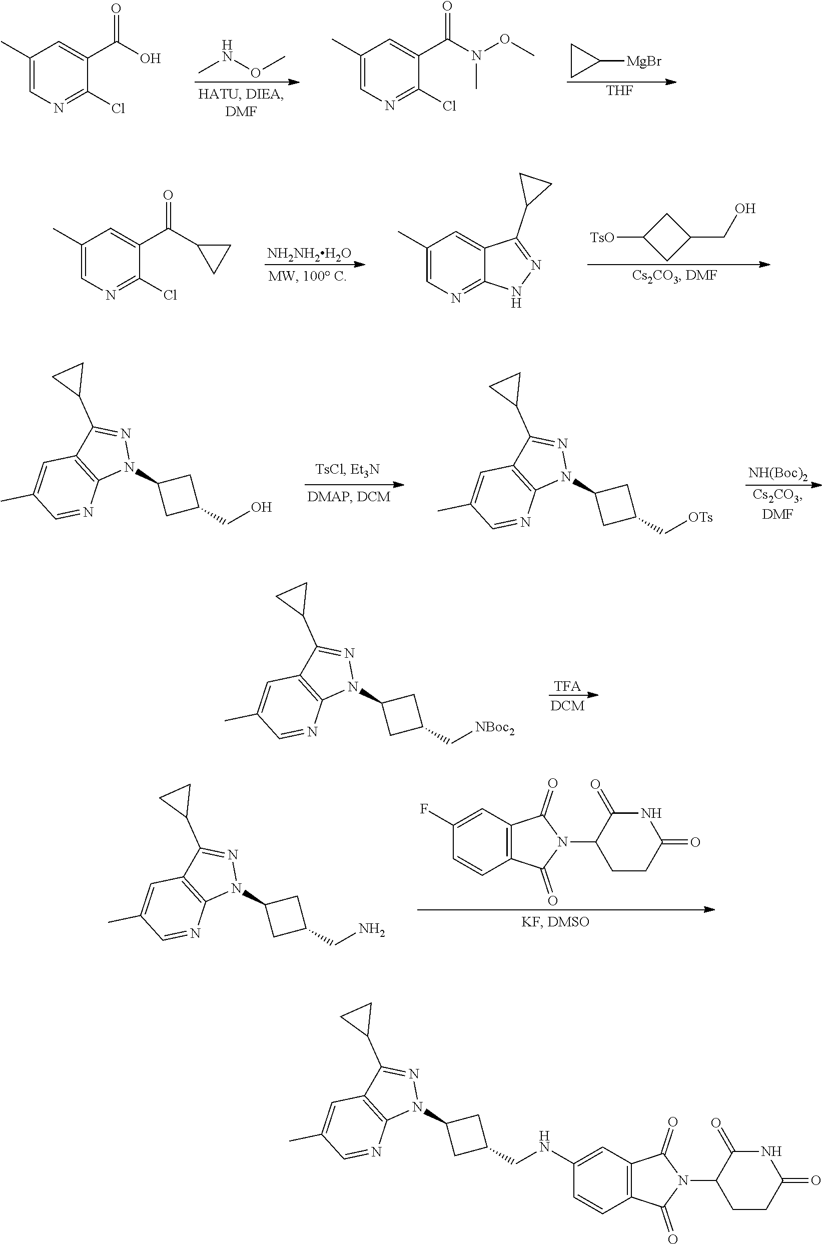

GS-673 was synthesized following the standard procedures for steps 1 to 3 GS-672, step 4 of GS-648 and steps 5 to 8 of GS-643 (8.9 mg, 1% yield over 7 steps) as a yellow solid. MS (ESI) m/z=513.5 [M+H]⁺.

### Example 35. 5-(((Trans-3-(3-cyclopropyl-6-methoxy-1H-pyrazolo[3,4-b]pyridin-1-yl)cyclobutyl)methyl)amino)-2-(2,6-dioxopiperidin-3-yl)isoindoline-1,3-dione (GS-674)

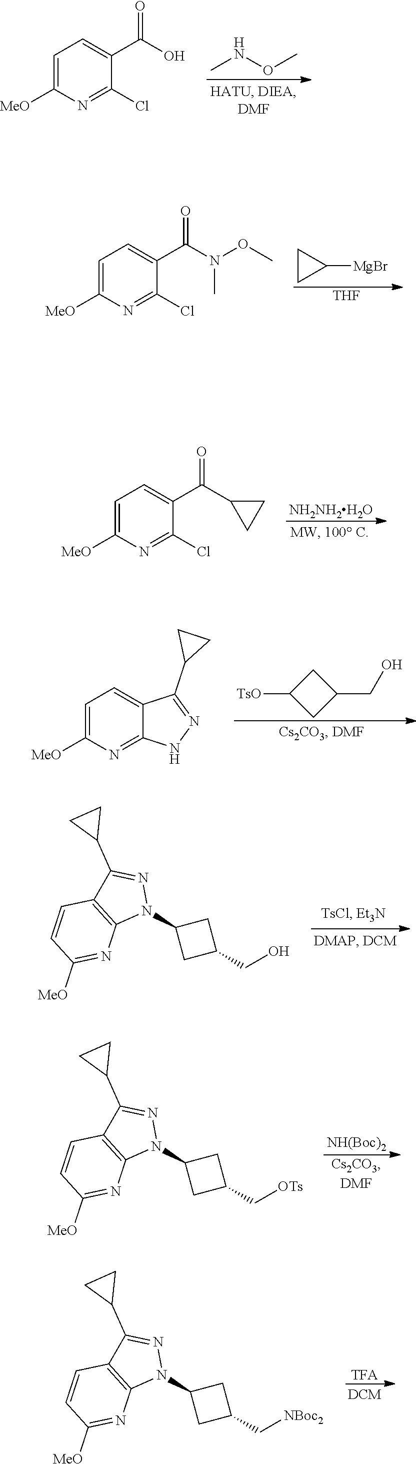

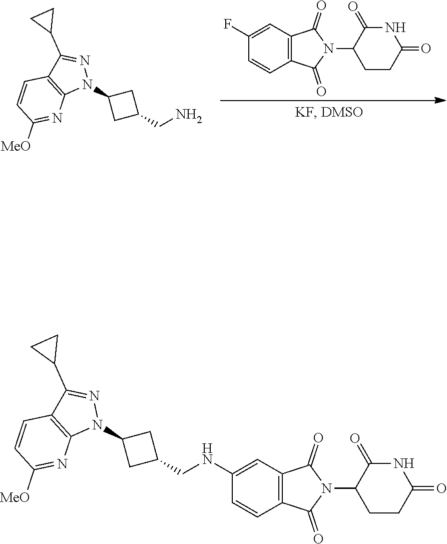 [SMILES: COc1ccc2c(C3CC3)nn([C@H]3C[C@H](CN)C3)c2n1 ; O=C1CCC(N2C(=O)c3ccc(F)cc3C2=O)C(=O)N1 ; COc1ccc2c(C3CC3)nn([C@H]3C[C@H](CNc4ccc5c(c4)C(=O)N(C4CCC(=O)NC4=O)C5=O)C3)c2n1]

GS-674 was synthesized following the standard procedures for steps 1 to 3 of GS-672, step 4 of GS-648 and step 5 to 8 of GS-643 (22.3 mg, 5% yield over 7 steps) as a yellow solid. MS (ESI) m/z=529.5 [M+H]⁺.

### Example 36. 5-(((Trans-3-(4-(1-(azetidin-3-yl)-1H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione (GS-675)

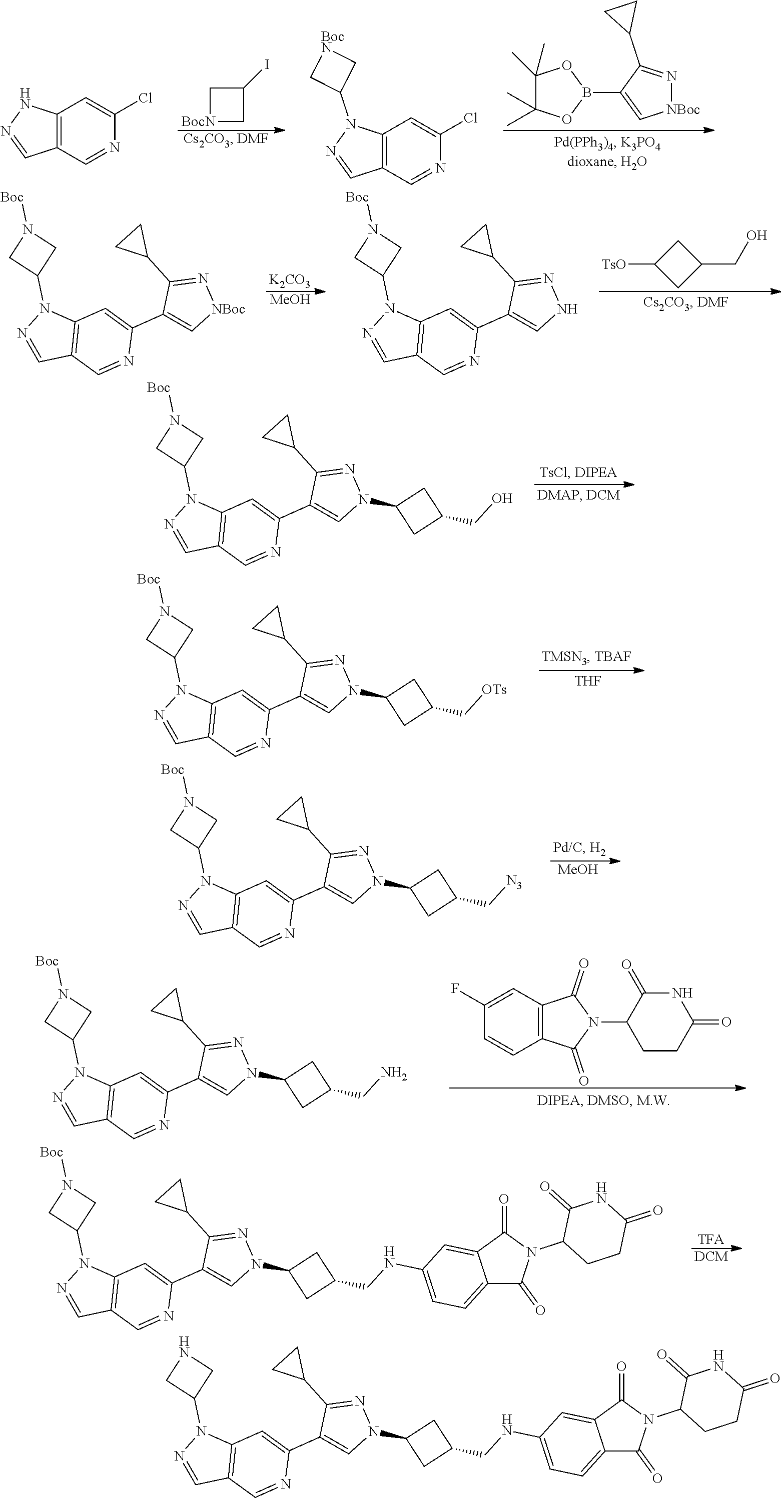

### Step 1. Synthesis of tert-butyl 3-(6-chloro-1H-pyrazolo[4,3-c]pyridin-1-yl)azetidine-1-carboxylate

To a solution of 6-chloro-1H-pyrazolo[4,3-c]pyridine (3 g, 19.54 mmol) and tert-butyl 3-iodoazetidine-1-carboxylate (6.08 g, 21.49 mmol) in DMF (30 mL) was added Cs₂CO₃ (12.73 g, 39.07 mmol) at rt. The reaction mixture was stirred at 90 [symbol] for 12 h. After cooling down to rt, the mixture was quenched with H₂O (100 mL) and extracted with ethyl acetate (100 mL×3). The combined organic phase was washed with brine, dried over anhydrous Na₂SO₄, filtered and concentrated under reduced pressure. The residue was purified by silica gel chromatography to give the desired product (1.7 g, 28% yield) as a yellow solid. MS (ESI) m/z=309.2 [M+H]⁺.

The remaining steps were performed according to the procedures for steps 2 to 9 of GS-665 to provide the desired product (1.3 mg, 1% yield over 8 steps) as a yellow solid. MS (ESI) m/z=620.5 [M+H]⁺.

### Example 37. 3-(5-(((Trans-3-(3-cyclopropyl-4-(5-fluoro-3-methylpyridin-2-yl)-1H-pyrazol-1-yl)cyclobutyl)methyl)amino)-1-oxoisoindolin-2-yl)piperidine-2,6-dione (GS-676)

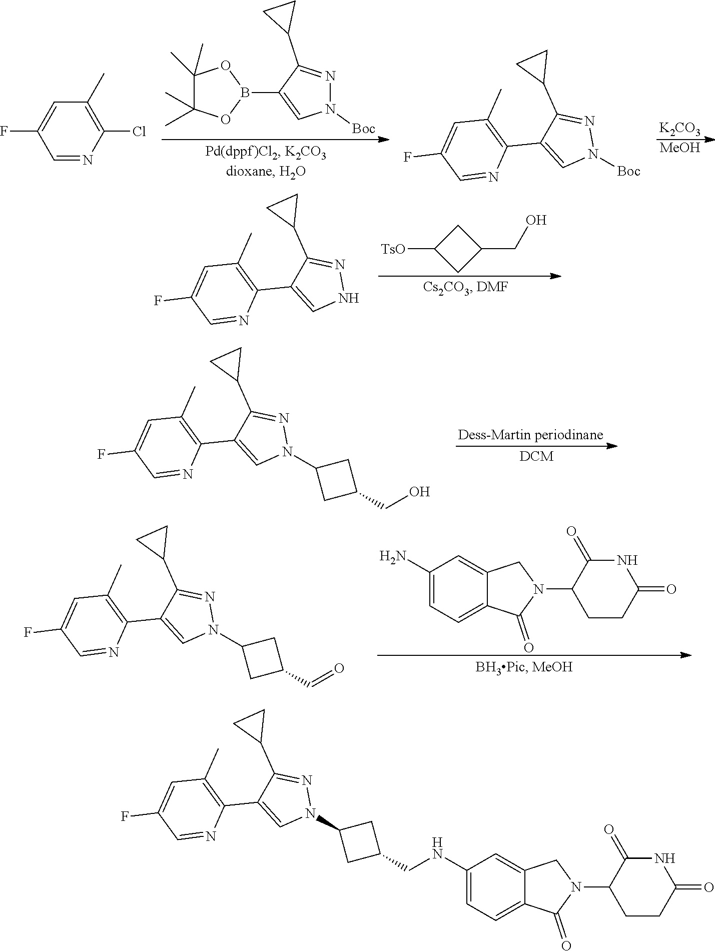

GS-676 was synthesized following the procedures for steps 1 to 4 of GS-678 (19 mg, 2% yield over 5 steps) as a yellow solid. MS (ESI) m/z=543.7 [M+H]⁺.

### Example 38. 3-(5-(((Trans-3-(3-(difluoromethyl)-4-(5-fluoro-6-methylpyridin-2-yl)-1H-pyrazol-1-yl)cyclobutyl)methyl)amino)-1-oxoisoindolin-2-yl)piperidine-2,6-dione (GS-677)

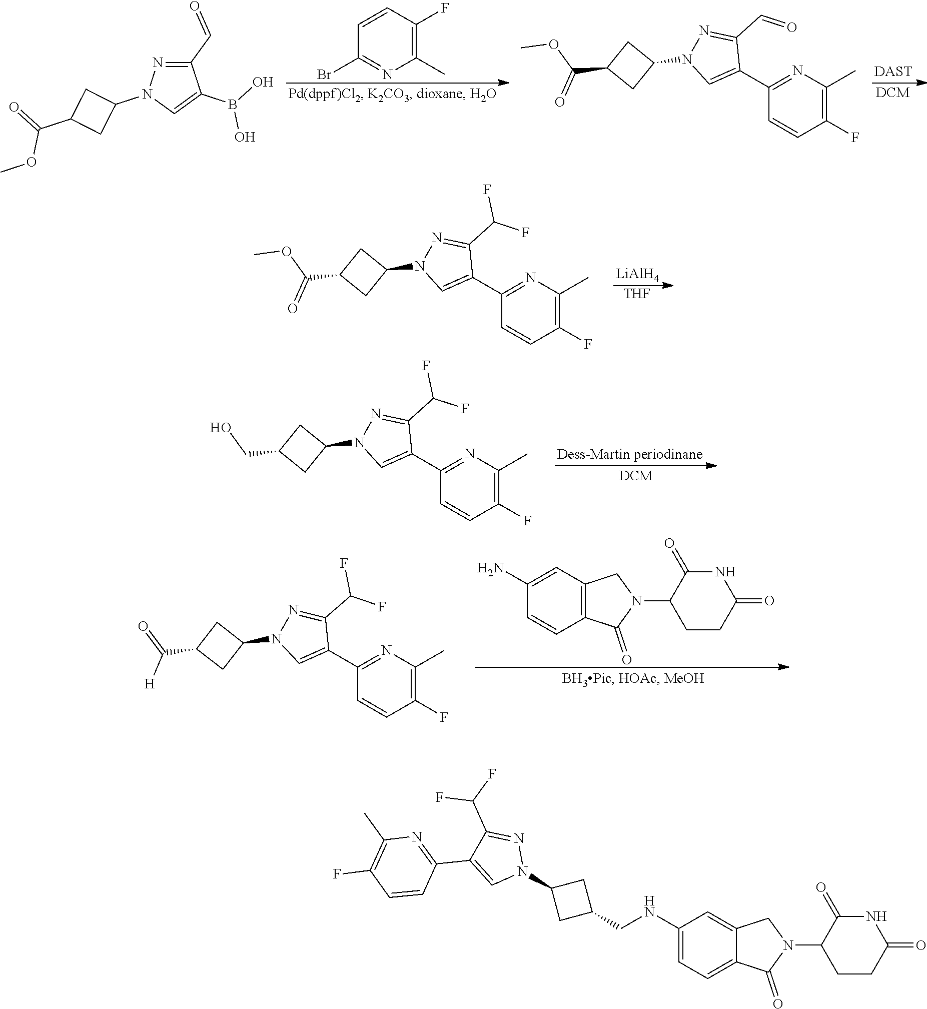

### Step 1. Synthesis of methyl trans-3-(4-(5-fluoro-6-methylpyridin-2 yl)-3-formyl-1H-pyrazol-1-yl)cyclobutane-1-carboxylate

To a solution of (3-formyl-1-(3-(methoxycarbonyl)cyclobutyl)-1H-pyrazol-4-yl)boronic acid (500 mg, 1.98 mmol) in dioxane (30 mL) and water (6 mL) were added 6-bromo-3-fluoro-2-methylpyridine (377 mg, 1.98 mmol), K₂CO₃ (820 mg, 5.95 mmol) and Pd(dppf)Cl₂ (145 mg, 0.19 mmol) at rt. The reaction mixture was stirred at 90° C. for 2 h under nitrogen. After cooling down to rt, the mixture was diluted with water (30 mL) and extracted with EtOAc (3×50 mL). The combined organic layers were washed with brine (50 mL), dried over Na₂SO₄, filtered and concentrated under reduced pressure. The residue was purified by silica gel column chromatography to provide the desired trans-isomer (180 mg, 28% yield) as a white solid. MS (ESI) m/z=318.4 [M+H]⁺.

### Step 2. Synthesis of methyl trans-3-(3-(difluoromethyl)-4-(5-fluoro-6-methylpyridin-2-yl)-1H-pyrazol-1-yl)cyclobutane-1-carboxylate

To a solution of methyl trans-3-(4-(5-fluoro-6-methylpyridin-2-yl)-3-formyl-1H-pyrazol-1-yl)cyclobutane-1-carboxylate (180 mg, 0.57 mmol) in dichloromethane (10 mL) was added DAST (914 mg, 5.67 mmol) at 0° C. The mixture was warmed to rt and stirred for 2 h, before it was quenched with aq. NaHCO₃ and extracted with dichloromethane (3×50 mL). The combined organic layers were washed with brine (50 mL), dried over Na₂SO₄, filtered and evaporated under reduced pressure. The residue was purified by silica gel column chromatography to provide the desired product (150 mg, 78% yield) as a white solid. MS (ESI) m/z=340.4 [M+H]⁺.

### Step 3. Synthesis of (trans-3-(3-(difluoromethyl)-4-(5-fluoro-6-methylpyridin-2-yl)-1H-pyrazol-1-yl)cyclobutyl)methanol

To a solution of methyl trans-3-(3-(difluoromethyl)-4-(5-fluoro-6-methylpyridin-2-yl)-1H-pyrazol-1-yl)cyclobutane-1-carboxylate (150 mg, 0.44 mmol) in THF (10 mL) was added LiAlH₄ (34 mg, 0.88 mmol). After the reaction mixture was stirred at 0° C. for 2 h under nitrogen, it was quenched with water and aq. NaOH (1N) carefully. The solid was filtered and the filtrate was extracted with ethyl acetate (3×20 mL). The combined organic layers were washed with brine (10 mL), dried over Na₂SO₄, filtered and evaporated under reduced pressure. The residue was purified by silica gel column chromatography to provide the desired product (110 mg, 80% yield) as a colorless oil. MS (ESI) m/z=312.4 [M+H]⁺.

### Step 4. Synthesis of trans-3-(3-(difluoromethyl)-4-(5-fluoro-6-methylpyridin-2-yl)-1H-pyrazol-1-yl)cyclobutane-1-carbaldehyde

To a solution of (trans-3-(3-(difluoromethyl)-4-(5-fluoro-6-methylpyridin-2-yl)-1H-pyrazol-1-yl)cyclobutyl)methanol (110 mg, 0.35 mmol) in dichloromethane (20 mL) was added Dess-Martin periodinane (450 mg, 1.06 mmol). The reaction mixture was stirred at 30° C. for 4 h, before it was quenched with aqueous sodium bicarbonate solution and extracted with dichloromethane (3×20 mL). The combined organic layers were washed with brine (20 mL), dried over Na₂SO₄, filtered and evaporated under reduced pressure. The residue was purified by silica gel column chromatography to provide the desired product (60 mg, 55% yield) as a colorless oil. MS (ESI) m/z=310.2 [M+H]⁺.

### Step 5. Synthesis of 3-(5-(((trans-3-(3-(difluoromethyl)-4-(5-fluoro-6-methylpyridin-2-yl)-1H-pyrazol-1-yl)cyclobutyl)methyl)amino)-1-oxoisoindolin-2-yl)piperidine-2,6-dione

To a solution of trans-3-(3-(difluoromethyl)-4-(5-fluoro-6-methylpyridin-2-yl)-1H-pyrazol-1-yl)cyclobutane-1-carbaldehyde (60 mg, 0.19 mmol) in MeOH (5 mL) were added 3-(5-amino-1-oxoisoindolin-2-yl)piperidine-2,6-dione (50 mg, 0.19 mmol) and borane-2-picoline complex (41 mg, 0.38 mmol) at 0° C. The reaction mixture was warmed to rt and stirred for 5 h. The mixture was quenched with water (20 mL) and extracted with ethyl acetate (3×20 mL). The combined organic layers were washed with saturated brine (20 mL), dried over anhydrous sodium sulfate, filtered and evaporated under reduced pressure. The residue was purified by reverse-phase chromatography to provide the desired product (11 mg, 10% yield) as a white solid. MS (ESI) m/z=553.5 [M+H]⁺.

### Example 39. 3-(5-(((Trans-3-(3-cyclopropyl-4-(1-methyl-1H-pyrazolo[4,3-c]pyridin-6-yl)-1H-pyrazol-1-yl)cyclobutyl)methyl)amino)-1-oxoisoindolin-2-yl)piperidine-2,6-dione (GS-678)

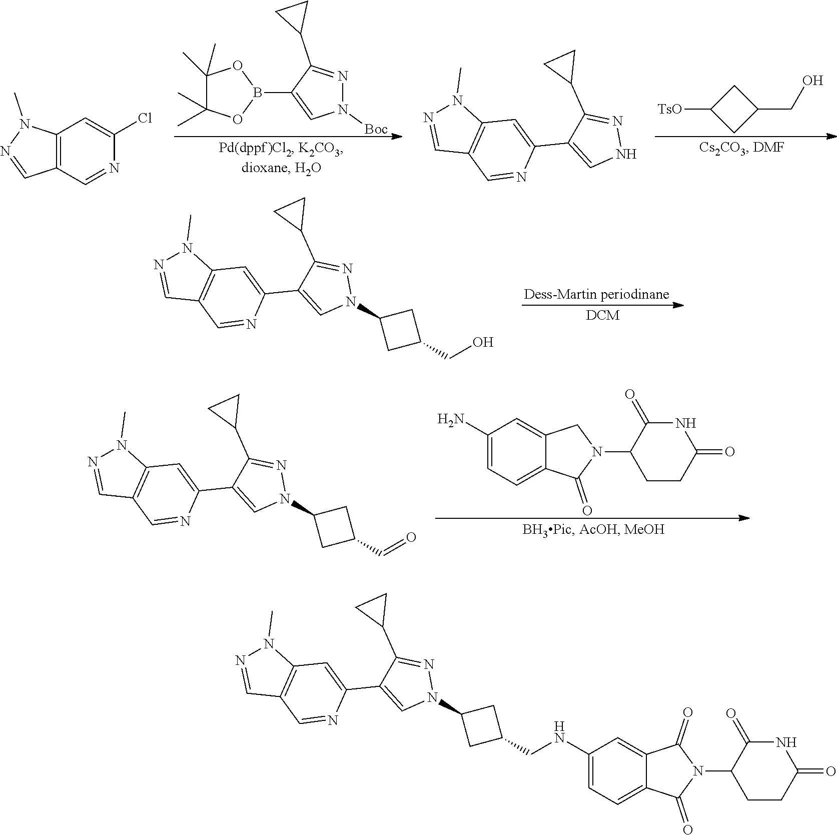

### Step 1. Synthesis of 6-(3-cyclopropyl-1H-pyrazol-4-yl)-1-methyl-1H-pyrazolo[4,3-c]pyridine

To a solution of 6-chloro-1-methyl-1H-pyrazolo[4,3-c]pyridine (3 g, 17.90 mmol) in dioxane (100 mL) and H₂O (20 mL) were added tert-butyl 3-cyclopropyl-4-(4,4,5,5-tetramethyl-1,3,2-dioxaborolan-2-yl)-1H-pyrazole-1-carboxylate (8.97 g, 26.85 mmol), Pd(dppf)Cl₂ (654.2 mg, 895.01 μmol) and K₂CO₃ (4.94 g, 35.80 mmol) at rt. The reaction mixture was stirred at 100 [symbol] for 6 h under inert atmosphere. After cooling down to rt, the mixture was poured into water (100 mL) an extracted with ethyl acetate (3×100 mL). The combined organic layers were washed with saturated brine (50 mL), dried over anhydrous sodium sulfate, filtered and evaporated under reduced pressure. The resulting residue was purified by silica gel chromatography to provide the desired product (2.5 g, 58% yield) as a pale-white solid. MS (ESI) m/z=240.4 [M+H]⁺.

### Step 2. Synthesis of (trans-3-(3-cyclopropyl-4-(1-methyl-1H-pyrazolo[4,3-c]pyridin-6-yl)-1H-pyrazol-1-yl)cyclobutyl)methanol

To a solution of 6-(3-cyclopropyl-1H-pyrazol-4-yl)-1-methyl-1H-pyrazolo[4,3-c]pyridine (2.5 g, 10.45 mmol) in DMF (20 mL) were added 3-(hydroxymethyl)cyclobutyl 4-methylbenzenesulfonate (3.21 g, 12.54 mmol) and Cs₂CO₃ (6.81 g, 20.90 mmol) at rt. The reaction mixture was stirred at 100 [symbol] for 3 h. After cooling down to rt, the mixture was poured into water (100 mL) and extracted with ethyl acetate (3×100 mL). The combined organic layers were washed with saturated brine (50 mL), dried over anhydrous sodium sulfate, filtered and evaporated under reduced pressure. The resulting residue was purified by silica gel chromatography to provide the desired product (1.1 g, 33% yield) as a pale-white solid. MS (ESI) m/z=324.3 [M+H]⁺.

### Step 3. Synthesis of trans-3-(3-cyclopropyl-4-(1-methyl-1H-pyrazolo[4,3-c]pyridin-6-yl)-1H-pyrazol-1-yl)cyclobutane-1-carbaldehyde

To a solution of (trans-3-(3-cyclopropyl-4-(1-methyl-1H-pyrazolo[4,3-c]pyridin-6-yl)-1H-pyrazol-1-yl)cyclobutyl)methanol (500 mg, 1.55 mmol) in dichloromethane (15 mL) was added Dess-Martin periodinane (786.6 mg, 1.86 mmol) at 0° C. in 3 portions. The reaction mixture was stirred at rt for 2 h, before it was poured into water (50 mL) and extracted with dichloromethane (3×50 mL). The combined organic layers were washed with saturated brine (50 mL), dried over anhydrous sodium sulfate, filtered and evaporated under reduced pressure. The resulting residue was purified by silica gel chromatography to provide the desired product (450 mg, 90% yield) as a pale-white solid. MS (ESI) m/z=322.5 [M+H]⁺.

### Step 4. Synthesis of 3-(5-(((trans-3-(3-cyclopropyl-4-(1-methyl-1H-pyrazolo[4,3-c]pyridin-6-yl)-1-pyrazol-1-yl)cyclobutyl)methyl)amino)-1-oxoisoindolin-2-yl)piperidine-2,6-dione

To a solution of trans-3-(3-cyclopropyl-4-(1-methyl-1H-pyrazolo[4,3-c]pyridin-6-yl)-1H-pyrazol-1-yl)cyclobutane-1-carbaldehyde (150 mg, 0.466 mmol) in MeOH (10 mL) were added 3-(5-amino-1-oxoisoindolin-2-yl)piperidine-2,6-dione (181.51 mg, 0.70 mmol), AcOH (1 mL) and borane-2-picoline complex (86.8 mg, 0.93 mmol) at 0° C. The reaction mixture was warmed to rt and stirred for 4 h. The mixture was quenched with water (20 mL) and extracted with ethyl acetate (3×50 mL). The combined organic layers were washed with saturated brine (50 mL), dried over anhydrous sodium sulfate, filtered and evaporated under reduced pressure. The residue was purified by silica gel chromatography to provide the desired product (120 mg, 46% yield) as a white solid. MS (ESI) m/z=565.4 [M+H]⁺.

### Example 40. 5-(((Trans-3-(3-cyclopropyl-4-methyl-1H-pyrazolo[3,4-b]pyridin-1-yl)cyclobutyl)methyl)amino)-2-(2,6-dioxopiperidin-3-yl)isoindoline-1,3-dione (GS-679)

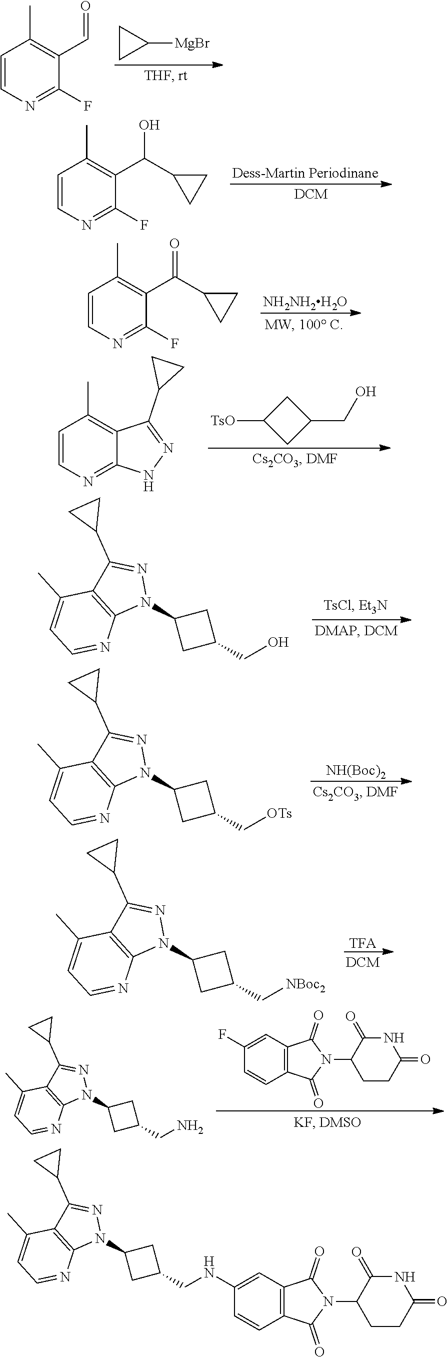

GS-679 was synthesized following the standard procedures for step 2 of GS-672, steps 2 to 3 of GS-663, step 4 of GS-648 and step 5 to 8 of GS-643 (5.8 mg, 1% yield over 7 steps) as a yellow solid. MS (ESI) m/z=513.5 [M+H]⁺.

### Example 41. 5-(((Trans-3-(3-cyclopropyl-4-(2-(1-methylazetidin-3-yl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-680)

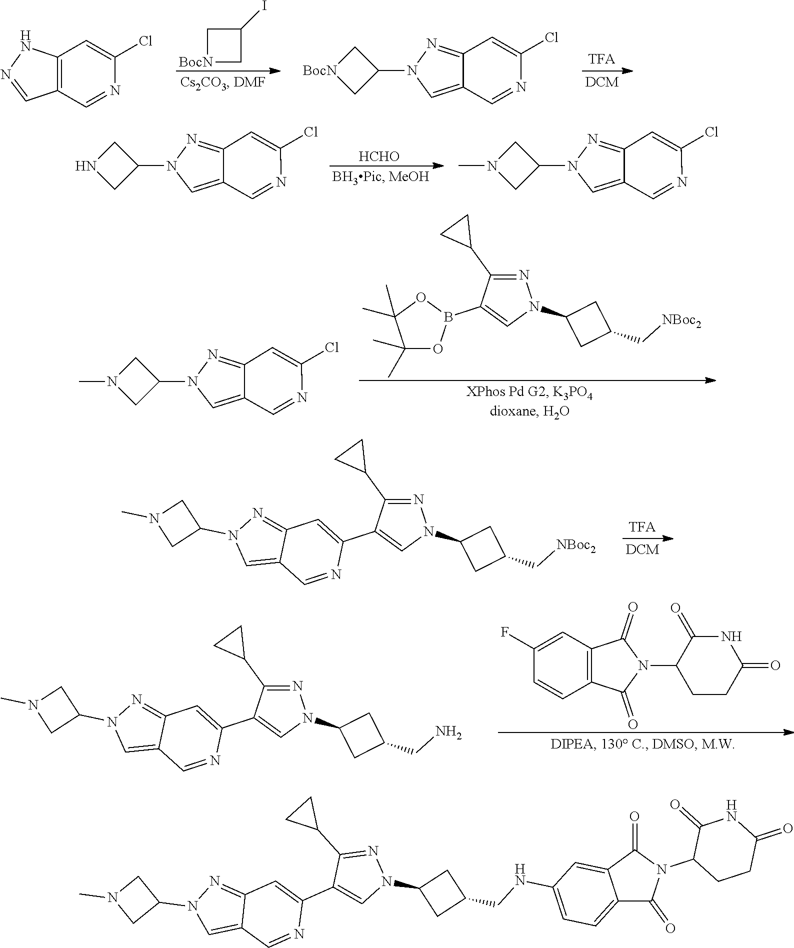

### Step 1. Synthesis of tert-butyl 3-(6-chloro-2H-pyrazolo[4,3-c]pyridin-2-yl)azetidine-1-carboxylate

To a solution of 6-chloro-11H-pyrazolo[4,3-c]pyridine (2.0 g, 13.02 mmol) and tert-butyl 3-iodoazetidine-1-carboxylate (5.53 g, 19.54 mmol) in DMF (30 mL) was added Cs₂CO₃ (8.49 g, 26.05 mmol). The reaction mixture was stirred at 90 [symbol] for 12 h. After cooling down to rt, the reaction mixture was poured into water (100 mL) and extracted with ethyl acetate (80 mL×3). The combined organic layers were washed with saturated brine (20 mL×2), dried over anhydrous sodium sulfate, filtered and evaporated under reduced pressure. The residue was purified by silica gel chromatography to provide the desired product (2.0 g, 50% yield) as a yellow solid. MS (ESI) m/z=309.2 [M+H]⁺.

### Step 2. Synthesis of 2-(azetidin-3-yl)-6-chloro-2H-pyrazolo[4,3-c]pyridine

To a solution of tert-butyl 3-(6-chloro-2H-pyrazolo[4,3-c]pyridin-2-yl)azetidine-1-carboxylate (1.0 g, 3.24 mmol) in DCM (10 mL) was added TFA (2 mL). The reaction mixture was stirred at 25 [symbol] for 1 h. The mixture was concentrated under vacuum to obtain the desired product (670 mg, 99% yield) as a yellow oil. MS (ESI) m/z=209.1 [M+H]⁺.

### Step 3. Synthesis of 6-chloro-2-(1-methylazetidin-3-yl)-2H-pyrazolo[4,3-c]pyridine

To a solution of 2-(azetidin-3-yl)-6-chloro-2H-pyrazolo[4,3-c]pyridine (670 mg, 3.2 mmol), two drops of acetic acid and formaldehyde solution (37 wt. % in H₂O, 482.1 mg, 16.0 mmol) in methanol (10 mL) was added 2-picoline borane complex (687 mg, 6.4 mmol). The reaction mixture was stirred at 25 [symbol] for 12 h. The mixture was quenched with H₂O (20 mL) and extracted with dichloromethane (20 mL×3). The combined organic phase was washed with brine (20 mL), dried over anhydrous Na₂SO₄, filtered and concentrated under reduced pressure. The residue was purified by silica gel chromatography to provide the desired product (600 mg, 84% yield) as a white solid. MS (ESI) m/z=223.1 [M+H]⁺.

### Step 4. Synthesis of tert-butyl N-tert-butoxycarbonyl-N-((trans-3-(3-cyclopropyl-4-(2-(1-methylazetidin-3-yl)pyrazolo[4,3-c]pyridin-6-yl)pyrazol-1-yl)cyclobutyl)methyl)carbamate

To a solution of 6-chloro-2-(1-methylazetidin-3-yl)-2H-pyrazolo[4,3-c]pyridine (250 mg, 1.12 mmol), tert-butyl N-tert-butoxycarbonyl-N-((trans-3-(3-cyclopropyl-4-(4,4,5,5-tetramethyl-1,3,2-dioxaborolan-2-yl)pyrazol-1-yl)cyclobutyl)methyl)carbamate (871.4 mg, 1.68 mmol) and K₃PO₄ (476.6 mg, 2.25 mmol) in dioxane (10 mL) and H₂O (2 mL) was added XPhos Pd G2 (88.2 mg, 0.11 mmol). The reaction mixture was stirred at 90 [symbol] for 12 h. After cooling down to rt, the mixture was quenched with H₂O (20 mL) and extracted with ethyl acetate (20 mL×3). The combined organic phase was washed with brine (20 mL), dried over anhydrous Na₂SO₄, filtered and concentrated under reduced pressure. The residue was purified by silica gel chromatography to provide the desired product (150 mg, 23% yield) as a yellow oil. MS (ESI) m/z=578.5 [M+H]⁺.

### Step 5. Synthesis of (trans-3-(3-cyclopropyl-4-(2-(1-methylazetidin-3-yl)-2H-pyrazolo[4,3-c]pyridin-6-yl)-1H-pyrazol-1-yl)cyclobutyl)methanamine

To a solution of tert-butyl N-tert-butoxycarbonyl-N-((trans-3-(3-cyclopropyl-4-(2-(1-methylazetidin-3-yl)pyrazolo[4,3-c]pyridin-6-yl)pyrazol-1-yl)cyclobutyl)methyl)carbamate (150 mg, 0.26 mmol) in DCM (10 mL) was added TFA (2 mL). The reaction mixture was stirred at 25 [symbol] for 1 h. The mixture was concentrated under vacuum to obtain the desired product (98 mg, 99% yield) as a yellow oil. MS (ESI) m/z=378.4 [M+H]⁺.

### Step 6. Synthesis of 5-(((trans-3-(3-cyclopropyl-4-(2-(1-methylazetidin-3-yl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

A mixture of (trans-3-(3-cyclopropyl-4-(2-(1-methylazetidin-3-yl)-2H-pyrazolo[4,3-c]pyridin-6-yl)-1H-pyrazol-1-yl)cyclobutyl)methanamine (98 mg, 0.26 mmol), 2-(2,6-dioxo-3-piperidyl)-5-fluoro-isoindoline-1,3-dione (73.2 mg, 0.26 mmol) and DIPEA (0.2 mL) in DMSO (1 mL) was heated at 130 [symbol] for 30 min under microwave irradiation. After cooling down to rt, the mixture was purified by reverse-phase chromatography to provide the desired product (2.0 mg, 1% yield) as a yellow solid. MS (ESI) m/z=634.5 [M+H]⁺.

### Example 42. 5-(((Trans-3-(3-cyclopropyl-4-methoxy-1H-pyrazolo[3,4-b]pyridin-1-yl)cyclobutyl)methyl)amino)-2-(2,6-dioxopiperidin-3-yl)isoindoline-1,3-dione (GS-681)

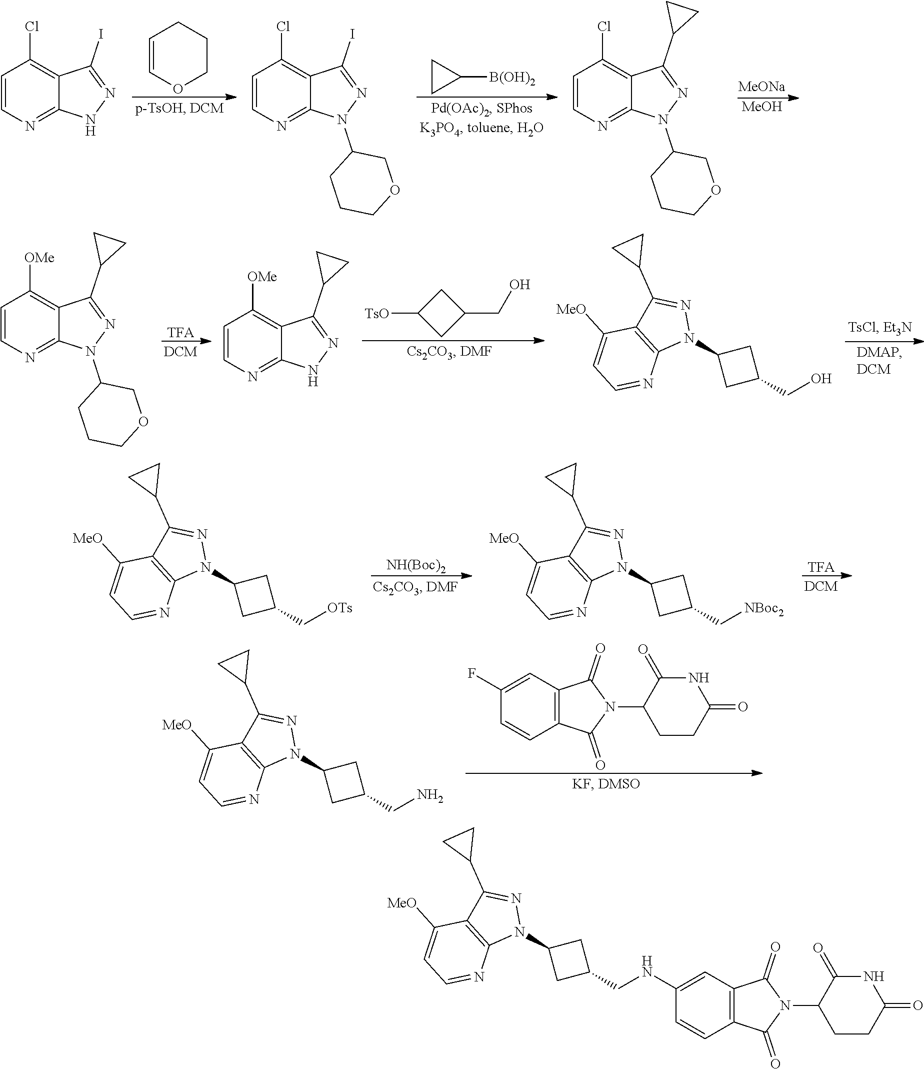

### Step 1. Synthesis of 4-chloro-3-iodo-1-(tetrahydro-2H-pyran-3-yl)-1H-pyrazolo[3,4-b]pyridine

To a mixture of 4-chloro-3-iodo-1H-pyrazolo[3,4-b]pyridine (16 g, 57.3 mmol) and 4-methylbenzenesulfonic acid (1.97 g, 11.5 mmol) in dichloromethane (150 mL) was added 3,4-dihydro-2H-pyran (9.63 g, 114.6 mmol) at rt. After the reaction mixture was stirred at rt overnight, it was quenched with water (100 mL) and extracted with dichloromethane (100 mL×3). The combined organic phase was washed with brine (50 mL), dried over anhydrous Na₂SO₄, filtered and concentrated under reduced pressure. The residue was purified by silica gel chromatography (petroleum ether/ethyl acetate=10:1) to provide the desired product (5.1 g, 24% yield) as a colorless oil. MS (ESI) m/z=364.1 [M+H]⁺.

### Step 2. Synthesis of 4-chloro-3-cyclopropyl-1-(tetrahydro-2H-pyran-3-yl)-1H-pyrazolo[3,4-b]pyridine

A mixture of 4-chloro-3-iodo-1-(tetrahydro-2H-pyran-2-yl)-1H-pyrazolo[3,4-b]pyridine (5 g, 13.8 mmol), cyclopropylboronic acid (1.72 g, 20.7 mmol), Pd(OAc)₂ (310.5 mg, 1.38 mmol), SPhos (1.13 g, 2.76 mmol) and K₃PO₄ (8.8 g, 41.4 mmol) in toluene (40 mL) and H₂O (4 mL) was stirred at 95 [symbol] for 12 h under nitrogen. After cooling down to rt, the reaction mixture was quenched with H₂O (10 mL) and extracted with ethyl acetate (30 mL×3). The combined organic phase was washed with brine (50 mL), dried over anhydrous Na₂SO₄, filtered and concentrated under reduced pressure. The residue was purified by silica gel chromatography (petroleum ether/ethyl acetate=10:1) to provide the desired product (2.32 g, 61% yield) as a white solid. MS (ESI) m/z=278.1 [M+H]⁺.

### Step 3. Synthesis of 3-cyclopropyl-4-methoxy-1-(tetrahydro-2H-pyran-3-yl)-1H-pyrazolo[3,4-b]pyridine

To a solution of 4-chloro-3-cyclopropyl-1-(tetrahydro-2H-pyran-2-yl)-1H-pyrazolo[3,4-b]pyridine (2.32 g, 8.38 mmol) in MeOH (20 mL) was added MeONa (2M, 8.38 mL, 16.8 mmol) at rt. The reaction mixture was heated at reflux for 3.5 h. The mixture was concentrated under reduced pressure. The residue was diluted with ethyl acetate (50 mL) and washed with brine (30 mL). The organic layer was dried over anhydrous Na₂SO₄, filtered and concentrated under reduced pressure. The residue was purified by silica gel chromatography (petroleum ether/ethyl acetate=10:1) to provide the desired product (1.65 g, 72% yield) as a white solid. MS (ESI) m/z=274.1 [M+H]⁺.

### Step 4. Synthesis of 3-cyclopropyl-4-methoxy-1H-pyrazolo[3,4-b]pyridine

To a solution of 3-cyclopropyl-4-methoxy-1-(tetrahydro-2H-pyran-2-yl)-1H-pyrazolo[3,4-b]pyridine (1.65 g, 6.04 mmol) in dichloromethane (15 mL) was added TFA (5 mL) at 0° C. After stirring at rt for 1 h, the reaction mixture was quenched with water (10 mL) and adjusted pH to 8 with sat. NaHCO₃. The mixture was extracted with dichloromethane (20 mL×3). The combined organic layers were dried over anhydrous Na₂SO₄, filtered and concentrated under reduced pressure. The residue was purified by silica gel chromatography (dichloromethane/MeOH=20:1) to provide the desired product (977 mg, 85% yield) as a white solid. ¹H NMR (400 MHz, DMSO-d₆) δ 12.98 (s, 1H), 8.30 (d, J=5.6 Hz, 1H), 6.65 (d, J=5.6 Hz, 1H), 4.00 (s, 3H), 2.42-2.36 (m, 1H), 0.97-0.89 (m, 4H). MS (ESI) m/z=190.1 [M+H]⁺.

The remaining steps were performed according to the procedures for step 4 of GS-648 and step 5 to 8 of GS-643 to provide the desired product (3.2 mg, 3% yield over 5 steps) as a yellow solid. MS (ESI) m/z=529.4 [M+H]⁺.

### Example 43. 5-(((Trans-3-(3-cyclopropyl-5-methoxy-1H-pyrazolo[3,4-b]pyridin-1-yl)cyclobutyl)methyl)amino)-2-(2,6-dioxopiperidin-3-yl)isoindoline-1,3-dione (GS-682)

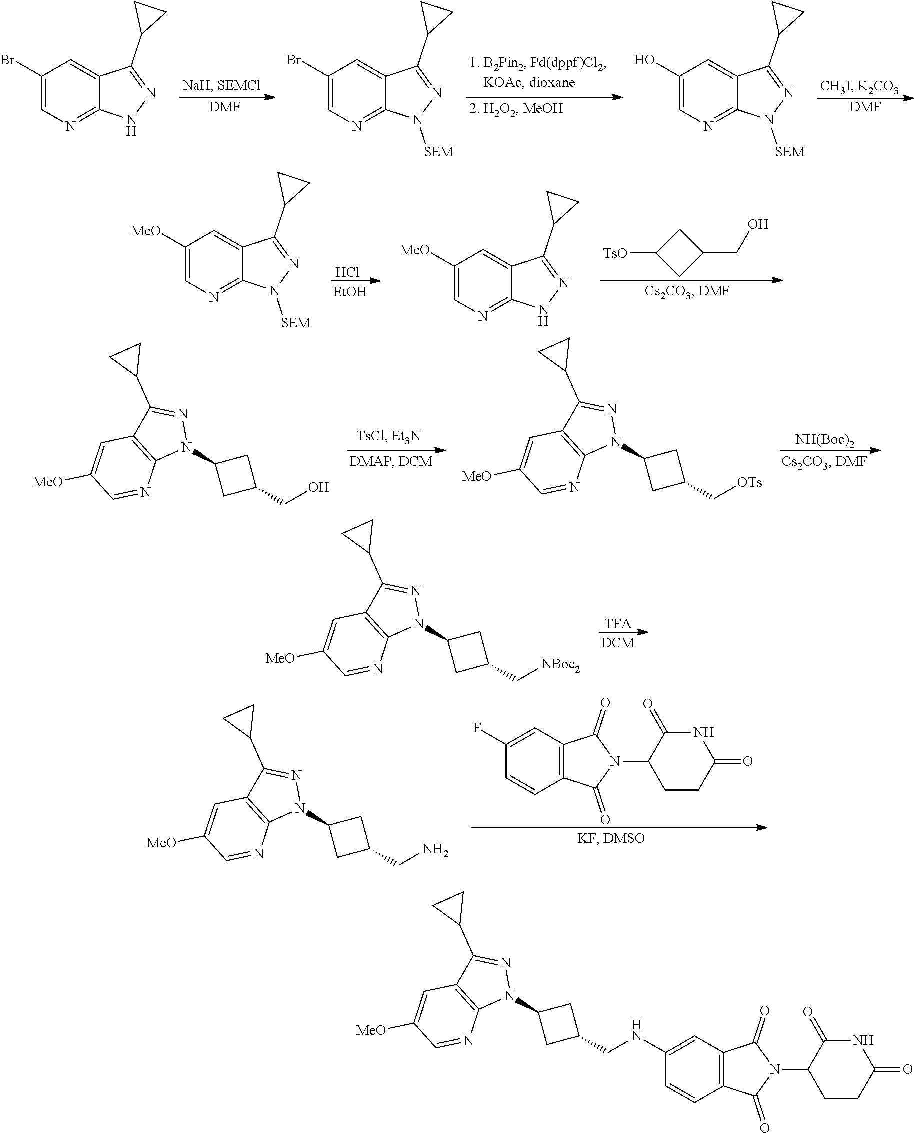

### Step 1. Synthesis of 5-bromo-3-cyclopropyl-1-((2-(trimethylsilyl)ethoxy)methyl)-1H-pyrazolo[3,4-b]pyridine

To a solution of 5-bromo-3-cyclopropyl-1H-pyrazolo[3,4-b]pyridine (4.12 g, 17.4 mmol) in DMF (30 mL) was added NaH (60% in oil, 695 mg, 17.4 mmol) at 0 [symbol] After the reaction was stirred at 0 [symbol] for 1 h, SEMCl (3.2 g, 19.1 mmol) was added dropwise. After the resulting mixture was stirred at 0 [symbol] for another 2 h, it was quenched with H₂O (15 mL) and extracted with EtOAc (50 mL×2). The combined organic layers were washed with brine (50 mL), dried over anhydrous Na₂SO₄, filtered and concentrated under reduced pressure. The residue was purified by silica gel chromatography (petroleum ether/ethyl acetate=10:1) to provide the desired product (5.7 g, 89% yield) as a white solid. MS (ESI) m/z=368.1 [M+H]⁺.

### Step 2. Synthesis of 3-cyclopropyl-1-((2-(trimethylsilyl)ethoxy)methyl)-1H-pyrazolo[3,4-b]pyridin-5-ol

To a solution of 5-bromo-3-cyclopropyl-1-((2-(trimethylsilyl)ethoxy)methyl)-1H-pyrazolo[3,4-b]pyridine (5.7 g, 15.5 mmol), bis(pinacolato)diboron (7.87 g, 31 mmol) in dioxane (50 mL) were added KOAc (3.04 g, 31 mmol) and Pd(dppf)Cl₂ (2.26 g, 3.1 mmol) at it. The reaction mixture was stirred at 80° C. overnight. After cooling down to rt, the reaction mixture was concentrated under reduced pressure. The residue was suspended in MeOH (50 mL) and filtered. To the filtrate was added H₂O₂ (20 mL), and the resulting solution was stirred at rt for 2 h. The mixture was diluted with ethyl acetate (100 mL), washed with brine (50 mL), dried over Na₂SO₄ and concentrated. The residue was purified by silica gel chromatography (petroleum ether/ethyl acetate=5:1) to provide the desired product (2.88 g, 61% yield) as a white solid. MS (ESI) m/z=306.1 [M+H]⁺.

### Step 3. Synthesis of 3-cyclopropyl-5-methoxy-1-((2-(trirmethylsilyl)ethoxy)methyl)-1H-pyrazolo[3,4-b]pyridine

To a solution of 3-cyclopropyl-1-((2-(trimethylsilyl)ethoxy)methyl)-1H-pyrazolo[3,4-b]pyridin-5-ol (2.88 g, 9.44 mmol) in DMF (20 mL) were added MeI (1.60 g, 11.33 mmol) and K₂CO₃ (2.60 g, 18.88 mmol). The reaction mixture was stirred at rt for 3 h. The mixture was poured into water (100 mL) and extracted with ethyl acetate (50 mL×3). The combined organic layers were washed with brine (50 mL), dried over anhydrous Na₂SO₄, filtered and concentrated under reduced pressure. The residue was purified by silica gel chromatography (petroleum ether/ethyl acetate=10:1) to provide the desired product (2.05 g, 68% yield) as a white solid. MS (ESI) m/z=320.1 [M+H]⁺

### Step 4. Synthesis of 3-cyclopropyl-5-methoxy-1H-pyrazolo[3,4-b]pyridine

To a stirred solution of 3-cyclopropyl-5-methoxy-1-((2-(trimethylsilyl)ethoxy)methyl)-1H-pyrazolo[3,4-b]pyridine (2.05 g, 6.4 mmol) in EtOH (15 mL) was added HCl (3N, 15 mL) at rt. The reaction mixture was stirred at 80° C. overnight. After cooling down to rt, the reaction mixture was poured into water (15 mL) and adjusted pH to 8 with sat. NaHCO₃. The mixture was extracted with dichloromethane (50 mL×3). The combined organic layers were washed with brine (50 mL), dried over anhydrous Na₂SO₄, filtered and concentrated under reduced pressure. The residue was purified by silica gel chromatography (dichloromethane/methanol=20:1) to provide the desired product (750 mg, 63% yield) as a white solid. ¹H NMR (400 MHz, DMSO-d₆) δ 12.97 (s, 1H), 8.23 (d, J=2.8 Hz, 1H), 7.71 (d, J=2.8 Hz, 1H), 3.86 (s, 3H), 2.30-2.24 (m, 1H), 1.00-0.92 (m, 4H). MS (ESI) m/z=190.1 [M+H]⁺.

The remaining steps were performed according to the procedures for step 4 of GS-648 and steps 5 to 8 of GS-643 to provide the desired product (9.7 mg, 4% yield over 5 steps) as a yellow solid. MS (ESI) m/z=529.4 [M+H]⁺.

### Example 44: 5-(((Trans-3-(3-cyclopropyl-4-(5-fluoro-3-(piperazin-1-yl)pyridin-2-yl)-1H-pyrazol-1-yl)cyclobutyl)methyl)amino)-2-(2,6-dioxopiperidin-3-yl)isoindoline-1,3-dione (GS-683)

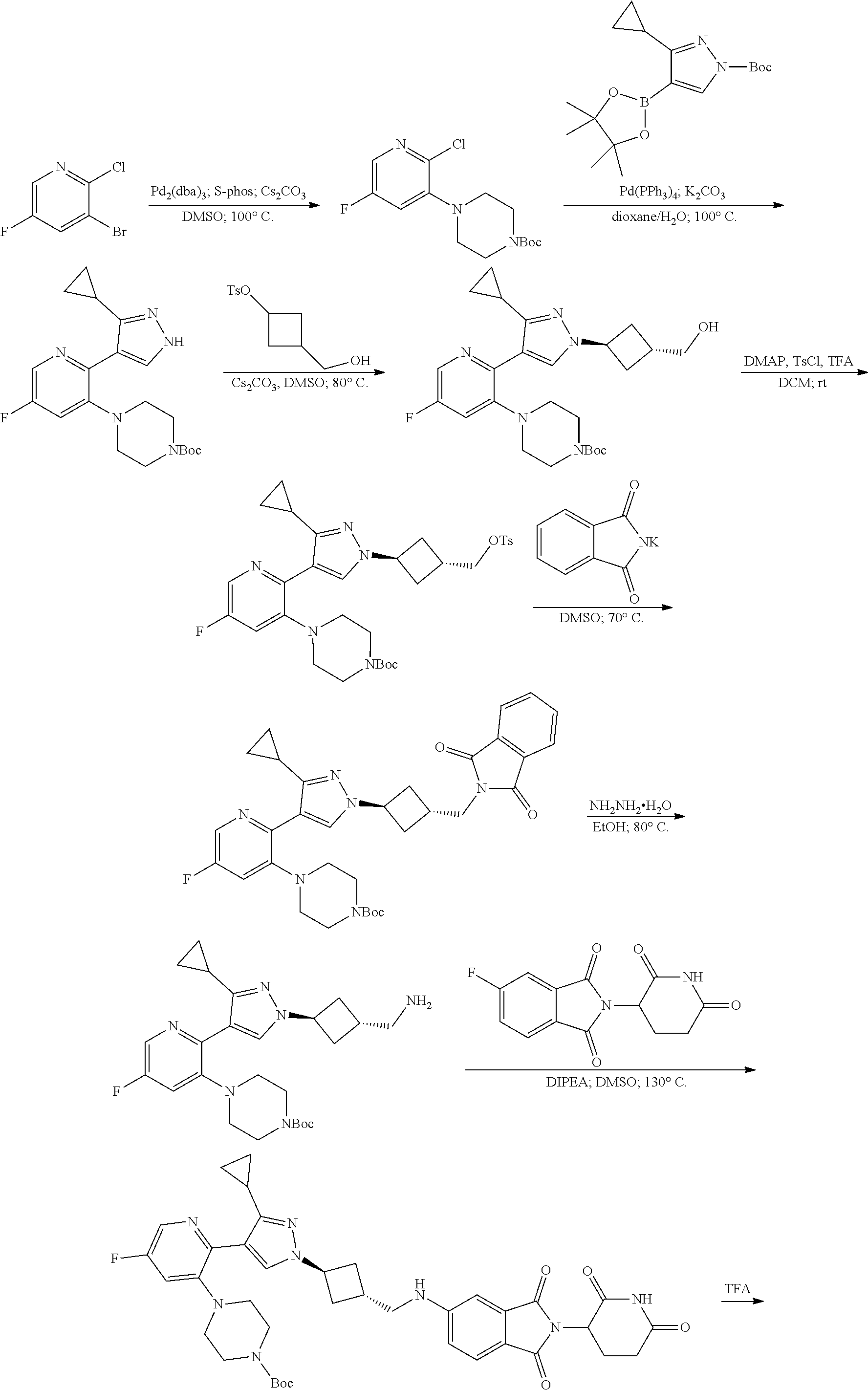

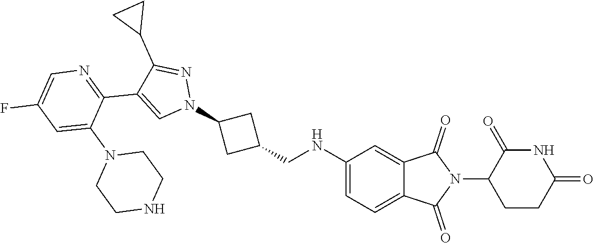 [SMILES: O=C1CCC(N2C(=O)c3ccc(NC[C@H]4C[C@H](n5cc(-c6ncc(F)cc6N6CCNCC6)c(C6CC6)n5)C4)cc3C2=O)C(=O)N1]

### Step 1. Synthesis of tert-butyl 4-(2-chloro-5-fluoropyridin-3-yl)piperazine-1-carboxylate

Under nitrogen atmosphere, a mixture of 3-bromo-2-chloro-5-fluoropyridine (41.6 mg, 0.2 mmol), tert-butyl piperazine-1-carboxylate (37.23 mg, 0.2 mmol), Pd₂(dba)₃ (18.3 mg, 0.02 mmol) and Cs₂CO₃ (195.6 mg, 0.6 mmol) in DMSO (3 mL) was stirred at 100° C. under nitrogen atmosphere for 12 h. The reaction mixture was diluted with EtOAc and washed with brine. The organic layer was concentrated and the residue was purified by flash column chromatography to provide the desired product (15.7 mg, yield: 25%) as a white solid. MS (ESI) m/z=315.8 [M+H]⁺.

### Step 2. Synthesis of tert-butyl 4-(2-(3-cyclopropyl-1H-pyrazol-4-yl)-5-fluoropyridin-3-yl)piperazine-1-carboxylate

Under nitrogen atmosphere, a mixture of tert-butyl 4-(2-chloro-5-fluoropyridin-3-yl)piperazine-1-carboxylate (1 g, 3.2 mmol), tert-butyl 3-cyclopropyl-4-(4,4,5,5-tetramethyl-1,3,2-dioxaborolan-2-yl)-1H-pyrazole-1-carboxylate (1.27 g, 3.8 mmol), Pd(PPh₃)₄ (349.8 mg, 0.32 mmol) and K₂CO₃ (1.33 g, 9.6 mmol) in 1,4-dioxane (10 mL) and H₂O (2 mL) was stirred at 100° C. under nitrogen atmosphere for 2 h. The reaction mixture was diluted with EtOAc and washed with brine. The organic layer was concentrated and the residue was purified by flash column chromatography to provide the desired product (300 mg, yield: 24%) as a white solid. MS (ESI) m/z=387.4 [M+H]⁺.

### Step 3. Synthesis of tert-butyl 4-(2-(3-cyclopropyl-1-(trans-3-(hydroxymethyl)cyclobutyl)-1H-pyrazol-4-yl)-5-fluoropyridin-3-yl)piperazine-1-carboxylate

To a solution of tert-butyl 4-(2-(3-cyclopropyl-1H-pyrazol-4-yl)-5-fluoropyridin-3-yl)piperazine-1-carboxylate (300 mg, 0.78 mmol) and 3-(hydroxymethyl)cyclobutyl 4-methylbenzenesulfonate (198 mg, 0.78 mmol) in DMSO (5 mL) was added Cs₂CO₃ (762.84 mg, 2.34 mmol). After the reaction was stirred at 100° C. for 2 h, it was cooled to rt and quenched with H₂O (100 mL). The mixture was extracted with EtOAc (30 mL×3). The organic layers were combined, dried, and concentrated. The residue was purified by flash column chromatography to provide the desired trans-isomer (160 mg, yield: 44%) as a white solid. MS (ESI) m/z=471.7 [M+H]⁺.

### Step 4. Synthesis of tert-butyl 4-(2-(3-cyclopropyl-1-(trans-3-((tosyloxy)methyl)cyclobutyl)-1H-pyrazol-4-yl)-5-fluoropyridin-3-yl)piperazine-1-carboxylate

To a solution of tert-butyl 4-(2-(3-cyclopropyl-1-(trans-3-(hydroxymethyl)cyclobutyl)-1H-pyrazol-4-yl)-5-fluoropyridin-3-yl)piperazine-1-carboxylate (160 mg, 0.34 mmol) and DMAP (4.14 mg, 0.034 mmol) in DCM (10 mL) were added TsCl (130 mg, 0.68 mmol) and TEA (171.7 mg, 1.7 mmol). After the reaction was stirred at 25 [symbol] for 1 h, it was diluted with DCM (20 mL) and washed with H₂O and brine. The organic layer was concentrated and the residue was purified flash column chromatography to provide the desired product (150 mg, yield: 70%) as a white solid. MS (ESI) m/z=625.8 [M+H]⁺.

### Step 5. Synthesis of tert-butyl 4-(2-(3-cyclopropyl-1-(trans-3-((1,3-dioxoisoindolin-2-yl)methyl)cyclobutyl)-1H-pyrazol-4-yl)-5-fluoropyridin-3-yl)piperazine-1-carboxylate

A mixture of tert-butyl 4-(2-(3-cyclopropyl-1-(trans-3-((tosyloxy)methyl)cyclobutyl)-1H-pyrazol-4-yl)-5-fluoropyridin-3-yl)piperazine-1-carboxylate (230 mg, 0.368 mmol) and potassium 1,3-dioxoisoindolin-2-ide (88.61 mg, 0.478 mmol) in DMSO (3 mL) was stirred 70 [symbol] for an hour. Then the mixture was diluted with DCM (10 mL) and washed with H₂O and brine. The organic layer was concentrated to provide the desired product (130 mg, yield: 59%) as a white solid. MS (ESI) m/z=600.7 [M+H]⁺.

### Step 6. Synthesis of tert-butyl 4-(2-(1-(trans-3-(aminomethyl)cyclobutyl)-3-cyclopropyl-1H-pyrazol-4-yl)-5-fluoropyridin-3-yl)piperazine-1-carboxylate

A mixture of tert-butyl 4-(2-(3-cyclopropyl-1-(trans-3-((1,3-dioxoisoindolin-2-yl)methyl)cyclobutyl)-1H-pyrazol-4-yl)-5-fluoropyridin-3-yl)piperazine-1-carboxylate (130 mg, 0.21 mmol) and N₂H₄ (3 mL; 80% in water) in EtOH (5 mL) was stirred at 80 [symbol] for an hour. Then the mixture was concentrated and the residue was purified by ISCO to provide the desired product (60 mg, yield: 61%) as a white solid. MS (ESI) m/z=470.7 [M+H]⁺.

### Step 7. Synthesis of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)-5-fluoropyridin-3-yl)piperazine-1-carboxylate

To a solution of tert-butyl 4-(2-(1-(trans-3-(aminomethyl)cyclobutyl)-3-cyclopropyl-1H-pyrazol-4-yl)-5-fluoropyridin-3-yl)piperazine-1-carboxylate (130 mg, 0.29 mmol) and 2-(2,6-dioxo-3-piperidyl)-5-fluoro-isoindoline-1,3-dione (118.99 mg, 0.43 mmol) in DMSO (2 mL) was added DIEA (371.2 mg, 2.9 mmol). After the reaction was stirred at 130 [symbol] in microwave for 2 h, it was purified by ISCO on C18 to provide the desired product (60 mg, yield: 28%) as a yellow solid. MS (ESI) m/z=726.8 [M+H]⁺.

### Step 8. Synthesis of 5-(((Trans-3-(3-cyclopropyl-4-(5-fluoro-3-(piperazin-1-yl)pyridin-2-yl)-1H-pyrazol-1-yl)cyclobutyl)methyl)amino)-2-(2,6-dioxopiperidin-3-yl)isoindoline-,3-dione

To a solution of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)-5-fluoropyridin-3-yl)piperazine-1-carboxylate (60 mg, 0.082 mmol) in DCM (3 mL) was added TFA (1 mL). After the reaction was stirred at the rt for 1 h, it was concentrated. The resulting residue was purified by prep-HPLC to provide the desired product (45 mg, yield: 88%) as a yellow solid. MS (ESI) m/z=626.8 [M+H]⁺.

### Example 45: 5-(((Trans-3-(3-cyclopropyl-4-(1H-pyrazolo[4,3-b]pyridin-5-yl)-1H-pyrazol-1-yl)cyclobutyl)methyl)amino)-2-(2,6-dioxopiperidin-3-yl)isoindoline-1,3-dione (GS-684)

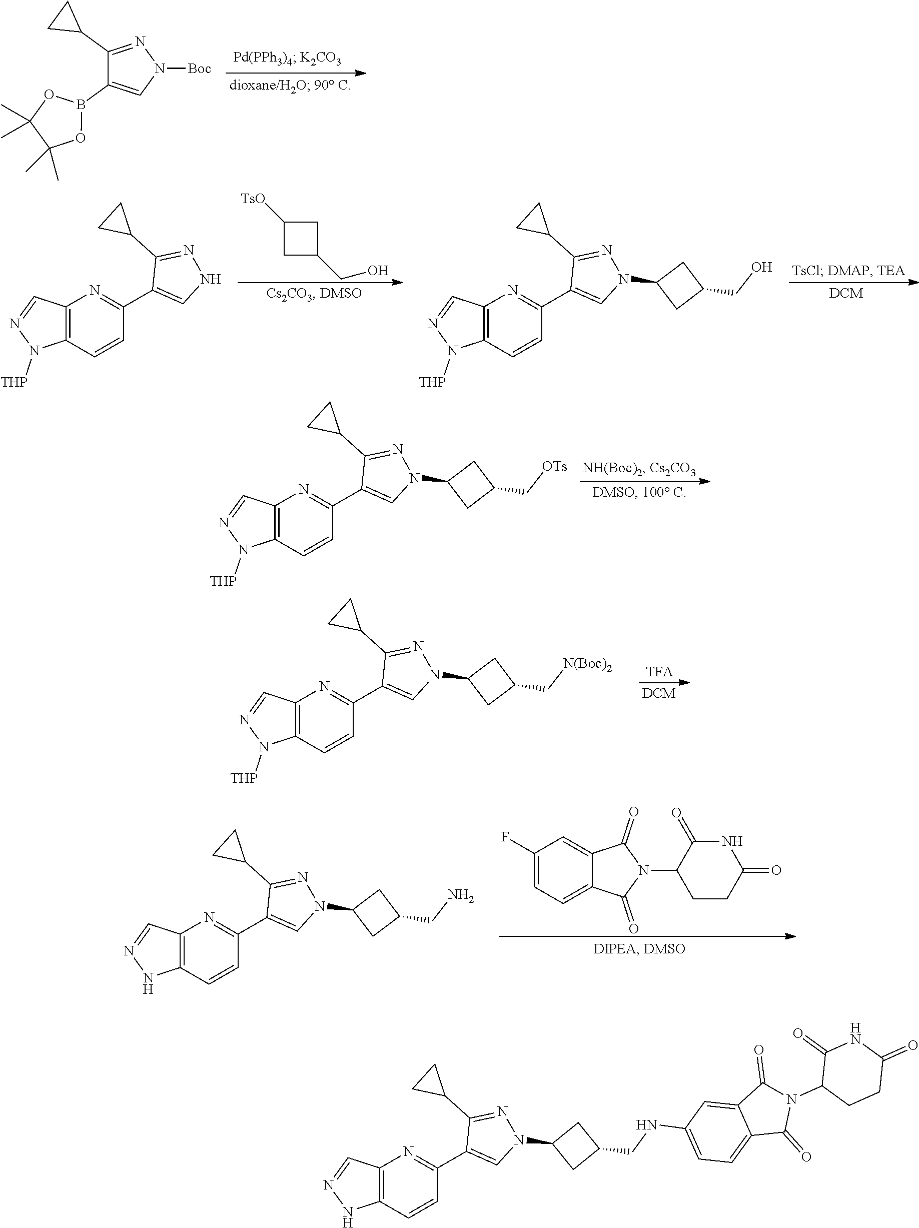

### Step 1. Synthesis of 5-(3-cyclopropyl-1H-pyrazol-4-yl)-1-(tetrahydro-2H-pyran-2-yl)-1H-pyrazolo[4,3-b]pyridine

To a solution of 5-chloro-1-(tetrahydro-2H-pyran-2-yl)-1H-pyrazolo[4,3-b]pyridine (500 mg, 2.1 mmol) in dioxane (10 mL) and water (2 mL) were added K₂CO₃ (871 mg, 6.3 mmol), Pd(dppf)Cl₂ (134 mg, 0.21 mmol) and tert-butyl 3-cyclopropyl-4-(4,4,5,5-tetramethyl-1,3,2-dioxaborolan-2-yl)-1H-pyrazole-1-carboxylate (843 mg, 2.5 mmol). After the mixture was stirred at 100 [symbol] for 12 h under N₂, it was purified by reverse phase chromatography (CH₃CN/H₂O/TFA) to provide the desired product (485 mg, yield: 75%) as a brown solid.

### Step 2. Synthesis of (trans-3-(3-cyclopropyl-4-(1-(tetrahydro-2H-pyran-2-yl)-1H-pyrazolo[4,3-b]pyridin-5-yl)-1H-pyrazol-1-yl)cyclobutyl)methanol

To a solution of 5-(3-cyclopropyl-1H-pyrazol-4-yl)-1-(tetrahydro-2H-pyran-2-yl)-1H-pyrazolo[4,3-b]pyridine (485 mg, 1.57 mmol) in DMSO (5 mL) were added Cs₂CO₃ (1.5 g, 4.71 mmol) and [3-(hydroxymethyl)cyclobutyl]4-methylbenzenesulfonate (486 mg, 1.88 mmol). The mixture was stirred at 100 [symbol] for 2.5 h under N₂. The mixture was poured into water and extracted with EtOAc (4×). The combined organic layers were washed with brine, dried, concentrated, and purified by silica gel flash chromatography to provide the title compound (270 mg, yield: 44%) as a white solid. MS (ESI) m/z=394.7 [M+H]⁺.

### Step 3. Synthesis of (trans-3-(3-cyclopropyl-4-(1-(tetrahydro-2H-pyran-2-yl)-1H-pyrazolo[4,3-b]pyridin-5-yl)-1H-pyrazol-1-yl)cyclobutyl)methyl 4-methylbenzenesulfonate

To a solution of (trans-3-(3-cyclopropyl-4-(1-(tetrahydro-2H-pyran-2-yl)-1H-pyrazolo[4,3-b]pyridin-5-yl)-1H-pyrazol-1-yl)cyclobutyl)methanol (270 mg, 0.69 mmol) in DCM (8 mL) were added DMAP (84 mg, 0.69 mmol), TEA (209 mg, 2.07 mmol) and 4-methylbenzenesulfonyl chloride (331 mg, 1.72 mmol). After the mixture was stirred at 25 [symbol] for 2 h, it was purified by silica gel flash chromatography to provide the title compound (270 mg, yield: 72%) as a white solid. MS (ESI) m/z=548.7 [M+H]⁺.

### Step 4. Synthesis of tert-butyl N-tert-butoxycarbonyl-N-[[trans-3-[3-cyclopropyl-4-(1-tetrahydropyran-2-ylpyrazolo[4,3-b]pyridin-5-yl)pyrazol-1-yl]cyclobutyl]methyl]carbamate

To a solution of (trans-3-(3-cyclopropyl-4-(1-(tetrahydro-2H-pyran-2-yl)-1H-pyrazolo[4,3-b]pyridin-5-yl)-1H-pyrazol-1-yl)cyclobutyl)methyl 4-methylbenzenesulfonate (270 mg, 0.49 mmol) in DMSO (5 mL) were added Cs₂CO₃ (479 mg, 1.47 mmol) and tert-butyl N-tert-butoxycarbonylcarbamate (139 mg, 0.64 mmol). The mixture was stirred at 90 [symbol] for 1.3 h under N₂, before it was purified by silica gel flash chromatography purification to provide the title compound (250 mg, yield: 86%) as a yellow solid. MS (ESI) m/z=593.8 [M+H]⁺.

### Step 5. Synthesis of (trans-3-(3-cyclopropyl-4-(1H-pyrazolo[4,3-b]pyridin-5-yl)-1H-pyrazol-1-yl)cyclobutyl)methanamine

A mixture of tert-butyl N-tert-butoxycarbonyl-N-[[trans-3-[3-cyclopropyl-4-(1-tetrahydropyran-2-ylpyrazolo[4,3-b]pyridin-5-yl)pyrazol-1-yl]cyclobutyl]methyl]carbamate (250 mg, 0.42 mmol) and TFA (1.5 mL) in DCM (3 mL) was stirred at rt for 1 h. The solvents was removed in vacuo to provide the desired product (120 mg, yield: 93%) as a white solid. MS (ESI) m/z=309.4 [M+H]⁺.

### Step 6. Synthesis of 5-(((Trans-3-(3-cyclopropyl-4-(1H-pyrazolo[4,3-b]pyridin-5-yl)-1H-pyrazol-1-yl)cyclobutyl)methyl)amino)-2-(2,6-dioxopiperidin-3-yl)isoindoline-1,3-dione

A mixture of (trans-3-(3-cyclopropyl-4-(1H-pyrazolo[4,3-b]pyridin-5-yl)-1H-pyrazol-1-yl)cyclobutyl)methanamine (60 mg, 0.19 mmol), 2-(2,6-dioxo-3-piperidyl)-5-fluoro-isoindoline-1,3-dione (54 mg, 0.19 mmol) and DIPEA (490 mg, 3.8 mmol) in DMSO (7 mL) was heated to 130 [symbol] for 2.5 h under microwave. The mixture was purified by reverse phase column purification (CH₃CN/H₂O/TFA) and prep-TLC (DCM:MeOH=18:1) to provide the title compound (25 mg, yield: 23%) as a yellow solid. MS (ESI) m/z=565.37 [M+H]⁺.

### Example 46: 5-((3-(Trans-3-(3-cyclopropyl-4-(1,2,3,4-tetrahydro-1,7-naphthyridin-8-yl)-1H-pyrazol-1-yl)cyclobutyl)propyl)amino)-2-(2,6-dioxopiperidin-3-yl)isoindoline-1,3-dione (GS-685)

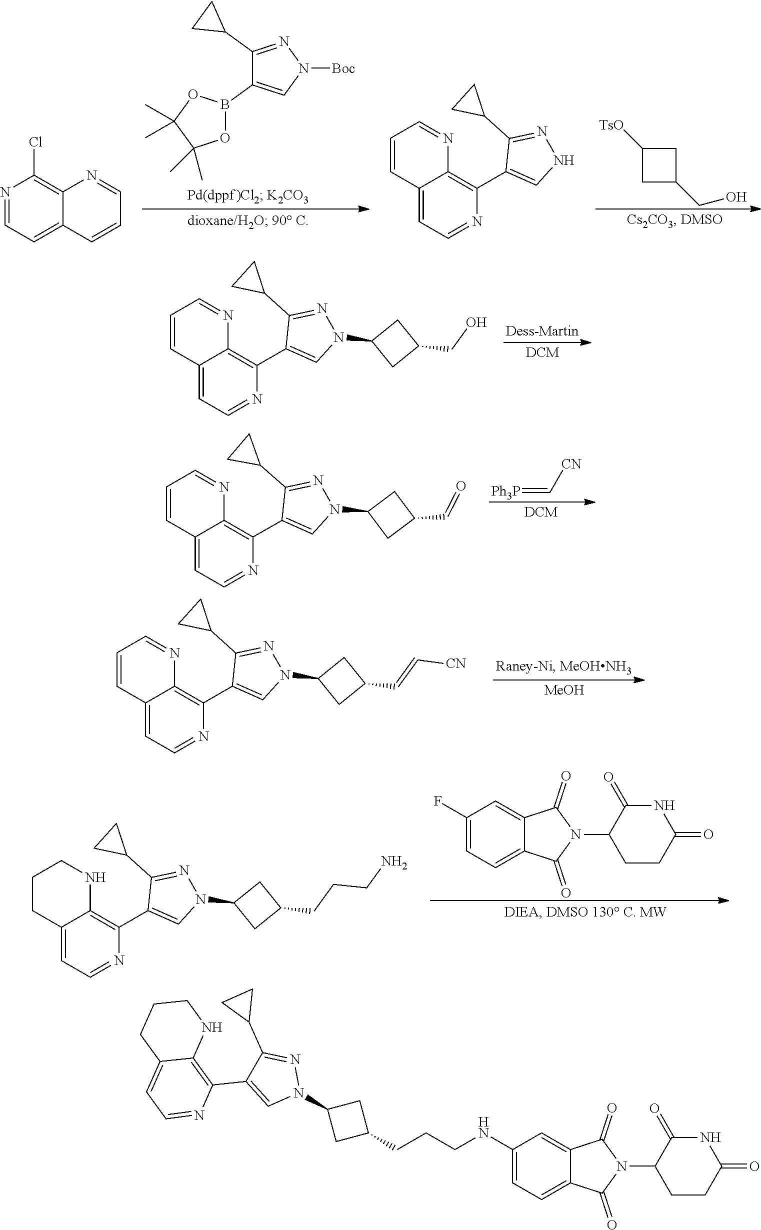

### Step 1. Synthesis of 8-(3-cyclopropyl-1H-pyrazol-4-yl)-1,7-naphthyridine

To a solution of 8-chloro-1,7-naphthyridine (500 mg, 3.04 mmol) in dioxane (15 mL) and H₂O (5 mL) were added Pd(dppf)Cl₂ (222.06 mg, 303.78 μmol), K₂CO₃ (2.10 g, 15.19 mmol) and tert-butyl 3-cyclopropyl-4-(4,4,5,5-tetramethyl-1,3,2-dioxaborolan-2-yl)-1H-pyrazole-1-carboxylate (1.12 g, 3.34 mmol). The mixture was stirred at 90 [symbol] for 12 h under N₂. The mixture was poured into water and extracted with a mixed solvent (EtOAc:MeOH=10:1). The organic layers were dried, concentrated, and purified by reverse phase column (MeOH/H₂O/TFA) to provide the desired product (348 mg, yield: 48%) as a black oil. MS (ESI) m/z=237.4 [M+H]⁺.

### Step 2. Synthesis of (trans-3-(3-cyclopropyl-4-(1,7-naphthyridin-8-yl)-1H-pyrazol-1-yl)cyclobutyl)methanol

To a solution of 8-(3-cyclopropyl-1H-pyrazol-4-yl)-1,7-naphthyridine (348 mg, 1.47 mmol) in DMF (5 mL) were added 3-(hydroxymethyl)cyclobutyl 4-methylbenzenesulfonate (679.55 mg, 2.65 mmol) and Cs₂CO₃ (1.44 g, 4.42 mmol). The mixture was stirred at 90 [symbol] for 6 h under N₂. The mixture was purified by reverse phase column (MeOH/H₂O/TFA) and prep-TLC (DCM:MeOH=12:1) to provide the desired trans-isomer (202 mg, yield: 43%) as a white solid. MS (ESI) m/z=321.6 [M+H]⁺.

### Step 3. Synthesis of trans-3-(3-cyclopropyl-4-(1,7-naphthyridin-8-yl)-1H-pyrazol-1-yl)cyclobutane-1-carbaldehyde

To a solution of (trans-3-(3-cyclopropyl-4-(1,7-naphthyridin-8-yl)-1H-pyrazol-1-yl)cyclobutyl)methanol (202 mg, 630.49 μmol) in DCM (5 mL) was added Dess-martin reagent (534.83 mg, 1.26 mmol). The reaction was stirred at rt under nitrogen atmosphere for 1 h. The mixture was filtered and filter cake was washed with DCM and EtOAc. The solvent was evaporated in vacuo to provide the title compound (190 mg, yield: 97%) as a crude brown oil. MS (ESI) m/z=319.4 [M+H]⁺.

### Step 4. Synthesis of (E)-3-(trans-3-(3-cyclopropyl-4-(1,7-naphthyridin-8-yl)-1H-pyrazol-1-yl)cyclobutyl)acrylonitrile

To a solution of trans-3-(3-cyclopropyl-4-(1,7-naphthyridin-8-yl)-1H-pyrazol-1-yl)cyclobutane-1-carbaldehyde (200 mg, 628.20 μmol) in DCM (8 mL) was added 2-(triphenyl-phosphanylidene)acetonitrile (340.72 mg, 1.13 mmol). After the mixture was stirred at rt for 0.5 h, the solvent was removed. The resulting residue was diluted with DMF and purified by reverse phase column purification (CH₃CN/H₂O/TFA) to provide the title compound (260 mg, yield: 91%) as a white solid. MS (ESI) m/z=342.5 [M+H]⁺.

### Step 5. Synthesis of 3-(trans-3-(3-cyclopropyl-4-(1,2,3,4-tetrahydro-1,7-naphthyridin-8-yl)-1H-pyrazol-1-yl)cyclobutyl)propan-1-amine

To a solution of (E)-3-(trans-3-(3-cyclopropyl-4-(1,7-naphthyridin-8-yl)-1H-pyrazol-1-yl)cyclobutyl)acrylonitrile (30 mg, 65.87 μmol) in MeOH (6 mL) were added NH₃-MeOH (0.5 mL) and Raney-Ni (60 mg). The reaction mixture was stirred at rt for 2 h under H₂. The reaction solution was filtered and concentrated to provide the title compound (20 mg, yield: 87%). MS (ESI) m/z=352.6 [M+H]⁺.

### Step 6. Synthesis of 5-((3-(Trans-3-(3-cyclopropyl-4-(1,2,3,4-tetrahydro-1,7-naphthyridin-8-yl)-1H-pyrazol-1-yl)cyclobutyl)propyl)amino)-2-(2,6-dioxopiperidin-3-yl)isoindoline-1,3-dione

A mixture of 3-(trans-3-(3-cyclopropyl-4-(1,2,3,4-tetrahydro-1,7-naphthyridin-8-yl)-1H-pyrazol-1-yl)cyclobutyl)propan-1-amine (19 mg, 0.054 mmol), 2-(2,6-dioxo-3-piperidyl)-5-fluoro-isoindoline-1,3-dione (22 mg, 0.081 mmol) and DIPEA (139 mg, 1.08 mmol) in DMSO (0.5 mL) was heated to 130 [symbol] for 2.5 h under microwave. The mixture was purified by reverse phase column purification (CH₃CN/H₂O/TFA) to provide the title compound (4.6 mg, yield: 14%) as a yellow solid.

### Example 47: 5-(((Trans-3-(3-cyclopropyl-4-(5-fluoro-3-(piperidin-4-yl)pyridin-2-yl)-1H-pyrazol-1-yl)cyclobutyl)methyl)amino)-2-(2,4-dioxocyclohexyl)isoindoline-1,3-dione (GS-686)

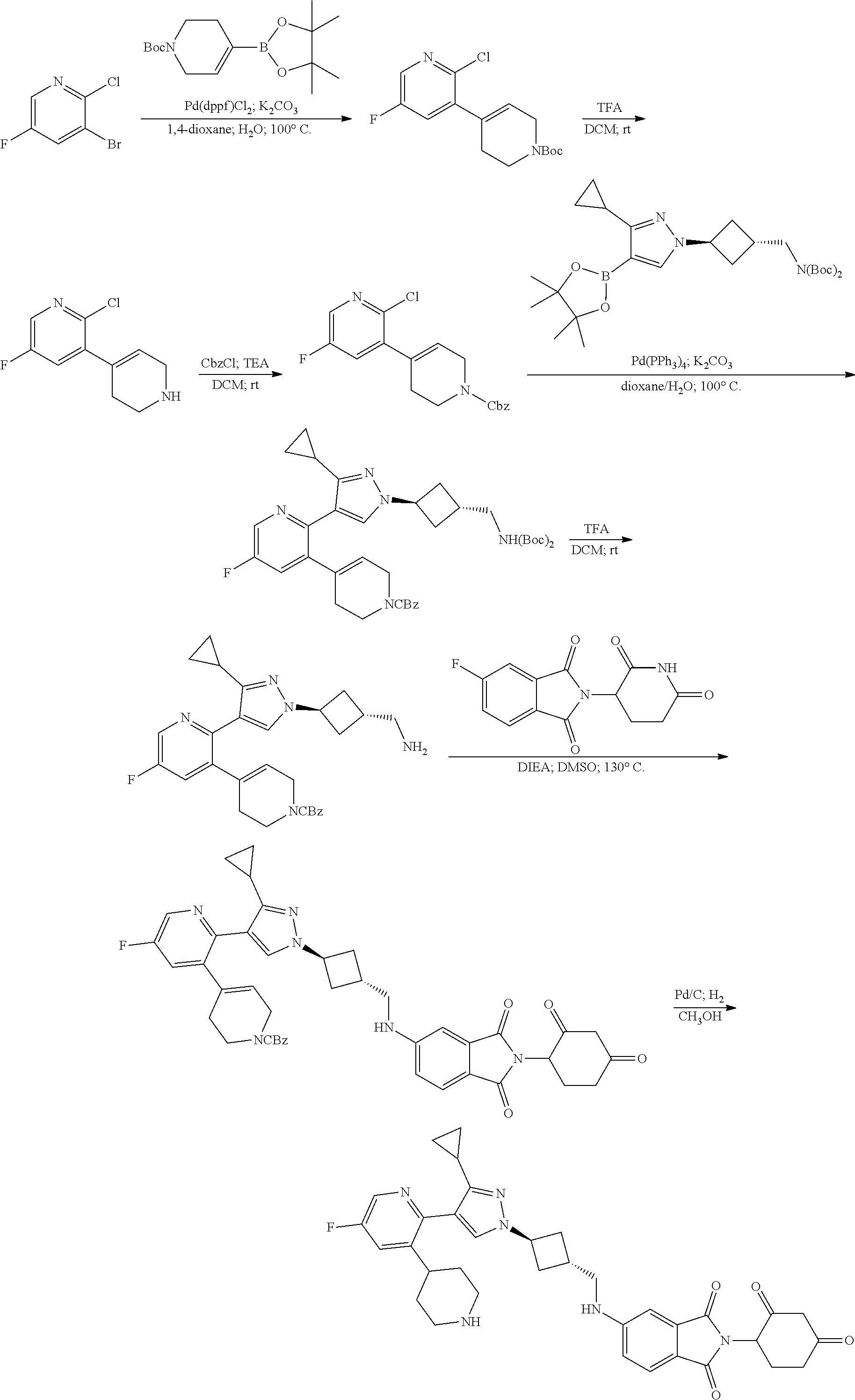

### Step 1. Synthesis of tert-butyl 2-chloro-5-fluoro-3′, 6′-dihydro-[3,4′-bipyridine]-1′(2′H)-carboxylate

A mixture of 3-bromo-2-chloro-5-fluoropyridine (1.0 g, 4.8 mmol), tert-butyl 4-(4,4,5,5-tetramethyl-1,3,2-dioxaborolan-2-yl)-3,6-dihydropyridine-1 (2H)-carboxylate (1.48 g, 4.8 mmol), Pd(dppf)Cl₂ (350.9 mg, 0.48 mmol) and K₂CO₃ (1.98 g, 14.44 mmol) in 1,4-dioxane (20 mL) and H₂O (4 mL) was stirred at 100 [symbol] for 2 h under N₂. The reaction was diluted with EtOAc (40 mL) and H₂O (20 mL). The organic layer was washed with brine, dried over anhydrous Na₂SO₄, and concentrated. The resulting residue was purified by flash column chromatography to provide the desired product (1.25 g, yield: 83%) as a white solid. MS (ESI) m/z=313.3 [M+H]⁺.

### Step 2. Synthesis of 2-chloro-5-fluoro-1′,2′,3′,6′-tetrahydro-3,4′-bipyridine

To a solution of tert-butyl 2-chloro-5-fluoro-3′,6′-dihydro-[3,4′-bipyridine]-1′(2′H)-carboxylate (1.4 g, 4.49 mmol) in DCM (20 mL) was added TFA (10 mL). After the reaction was stirred at rt for 30 min, it was concentrated to provide the desired product (1.46 g, yield: 95%) as a white solid. MS (ESI) m/z=212.6 [M+H]⁺.

### Step 3. Synthesis of benzyl 2-chloro-5-fluoro-3′,6′-dihydro-[3,4′-bipyridine]-1′(2′H)-carboxylate

To a solution of 2-chloro-5-fluoro-1′,2′,3′,6′-tetrahydro-3,4′-bipyridine (522.9 mg, 2.46 mmol) and TEA (3 mL) in DCM (10 mL) was added CbzCl (838.9 mg, 4.93 mmol) dropwise under nitrogen atmosphere at 0 [symbol] . After the reaction was stirred at rt for 2 h, it was concentrated. The residue was purified by flash column chromatography to provide the desired product (700 mg, yield: 82%) as a white solid. MS (ESI) m/z=346.4 [M+H]⁺.

### Step 4. Synthesis of benzyl 4-[2-[1-[trans-3-[[bis(tert-butoxycarbonyl)amino]methyl]cyclobutyl]-3-cyclopropyl-pyrazol-4-yl]-5-fluoro-3-pyridyl]-3,6-dihydro-2H-pyridine-1-carboxylate

A mixture of benzyl 2-chloro-5-fluoro-3′,6′-dihydro-[3,4′-bipyridine]-1′(2′H)-carboxylate (300 mg, 0.87 mmol), tert-butyl N-tert-butoxycarbonyl-N-[[trans-3-[3-cyclopropyl-4-(4,4,5,5-tetramethyl-1,3,2-dioxaborolan-2-yl)pyrazol-1-yl]cyclobutyl]methyl]carbamate (675.2 mg, 1.31 mmol), Pd(PPh₃)₄ (100.53 mg, 0.087 mmol) and K₂CO₃ (360.18 mg, 2.61 mmol) in 1,4-dioxane (10 mL) and H₂O (2.5 mL) was stirred at 100 [symbol] for 15 h. The reaction mixture was diluted with EtOAc and washed with H₂O and brine. The organic layer was concentrated and the residue was purified by flash column chromatography to provide the desired product (92 mg, yield: 10%) as a white solid. MS (ESI) m/z=702.8 [M+H]⁺.

### Step 5. Synthesis of benzyl 2-(1-(trans-3-(aminomethyl)cyclobutyl)-3-cyclopropyl-1H-pyrazol-4-yl)-5-fluoro-3′, 6′-dihydro-[3,4′-bipyridine]-1′(2′H)-carboxylate

To a solution of benzyl 4-[2-[1-[trans-3-[[bis(tert-butoxycarbonyl)amino]methyl]cyclobutyl]-3-cyclopropyl-pyrazol-4-yl]-5-fluoro-3-pyridyl]-3,6-dihydro-2H-pyridine-1-carboxylate (92 mg, 0.13 mmol) in DCM (10 mL) was added TFA (5 mL). After the reaction was stirred at 25 [symbol] for 1 h, it was concentrated to provide the title compound (70 mg, yield: 99%) as a white solid. MS (ESI) m/z=502.8 [M+H]⁺.

### Step 6. Synthesis of benzyl 2-(3-cyclopropyl-1-(trans-3-(((2-(2,4-dioxocyclohexyl)-1,3-dioxoisoindolin-5-yl)amino)methyl)cyclobutyl)-1H-pyrazol-4-yl)-5-fluoro-3′,6′-dihydro-[3,4′-bipyridine]-1′(2′H)-carboxylate

To a solution of benzyl 2-(1-(trans-3-(aminomethyl)cyclobutyl)-3-cyclopropyl-1H-pyrazol-4-yl)-5-fluoro-3′,6′-dihydro-[3,4′-bipyridine]-1′(2′H)-carboxylate (70 mg, 0.13 mmol), and 2-(2,6-dioxo-3-piperidyl)-5-fluoro-isoindoline-1,3-dione (35.88 mg, 0.13 mmol) in DMSO (2 mL) was added DIEA (166.4 mg, 1.3 mmol) at rt. After the reaction was stirred at 130 [symbol] for 1 h under microwave irradiation, it was purified by ISCO to provide the desired product (35 mg, yield: 36%) as a yellow solid. MS (ESI) m/z=756.7 [M+H]⁺.

### Step 7. Synthesis of 5-(((trans-3-(3-cyclopropyl-4-(5-fluoro-3-(piperidin-4-yl)pyridin-2-yl)-1H-pyrazol-1-yl)cyclobutyl)methyl)amino)-2-(2,4-dioxocyclohexyl)isoindoline-1,3-dione

A mixture of benzyl 2-(3-cyclopropyl-1-(trans-3-(((2-(2,4-dioxocyclohexyl)-1,3-dioxoisoindolin-5-yl)amino)methyl)cyclobutyl)-1H-pyrazol-4-yl)-5-fluoro-3′,6′-dihydro-[3,4′-bipyridine]-1′(2′H)-carboxylate (35 mg, 0.046 mmol) and Pd/C (10 mg, 10%) in EtOH (5 mL) was stirred at 25 [symbol] for 1 h under H₂ atmosphere. Then the mixture was filtered and the filtrate was concentrated. The residue was purified by prep-TLC to provide the desired product (14 mg, yield: 49%) as a yellow solid. MS (ESI) m/z=624.7 [M+H]⁺.

### Example 48: 5-(((Trans-3-(3-cyclopropyl-4-(1-methyl-1H-imidazol-2-yl)-1H-pyrazol-1-yl)cyclobutyl)methyl)amino)-2-(2,6-dioxopiperidin-3-yl)isoindoline-1,3-dione (GS-687)

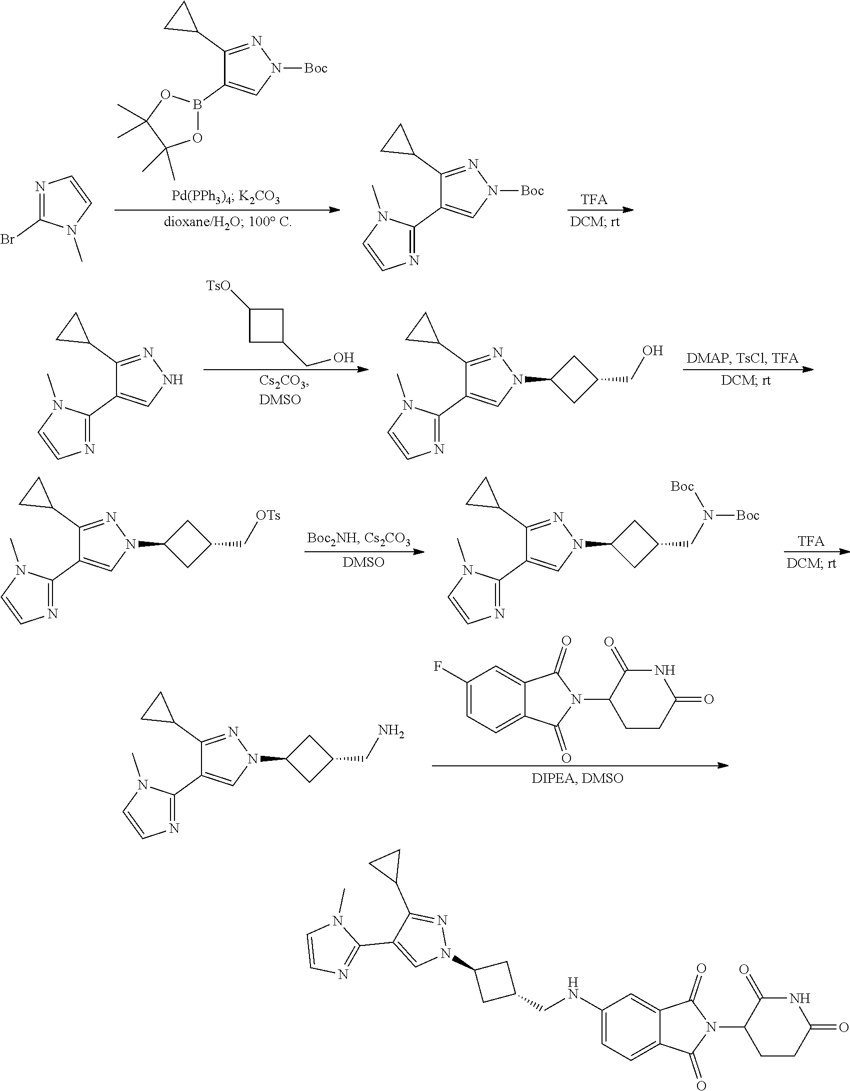

### Step 1. Synthesis of tert-butyl 3-cyclopropyl-4-(1-methyl-1H-imidazol-2-yl)-1H-pyrazole-1-carboxylate

A mixture of 2-bromo-1-methyl-1H-imidazole (350 mg, 2.17 mmol), tert-butyl 3-cyclopropyl-4-(4,4,5,5-tetramethyl-1,3,2-dioxaborolan-2-yl)-1H-pyrazole-1-carboxylate (943.9 mg, 2.83 mmol), Pd(PPh₃)₄ (250.74 mg, 0.217 mmol) and K₂CO₃ (898.4 mg, 6.51 mmol) in 1,4-dioxane (10 mL) and H₂O (2.5 mL) was stirred at 100 [symbol] under N₂ for 2 h. The reaction mixture was diluted with EtOAc and washed with H₂O and brine. The organic layer was concentrated and the residue was purified by flash column chromatography to provide the desired product (250 mg, yield: 40%) as a white solid. MS (ESI) m/z=288.3 [M+H]⁺.

### Step 2. Synthesis of 3-cyclopropyl-4-(1-methyl-1H-imidazol-2-yl)-1H-pyrazole

To a solution of tert-butyl 3-cyclopropyl-4-(1-methyl-1H-imidazol-2-yl)-1H-pyrazole-1-carboxylate (250 mg, 0.87 mmol) in DCM (10 mL) was added TFA (5 mL). After the reaction was stirred at 25 [symbol] for 1 h. The reaction mixture was concentrated to provide the desired product (167 mg, yield: 99%) as a white solid. MS (ESI) m/z=188.3 [M+H]⁺.

The remaining steps were performed according to the procedures for steps 2 to 6 GS-684 to provide the desired product (12 mg, yield: 6% over 5 steps) as a yellow solid. MS (ESI) m/z=528.7 [M+H]⁺.

### Example 49: 2-(2,6-Dioxopiperidin-3-yl)-5-(((trans-3-(3-methyl-2H-indazol-2-yl)cyclobutyl)methyl)amino)isoindoline-1,3-dione (GS-688)

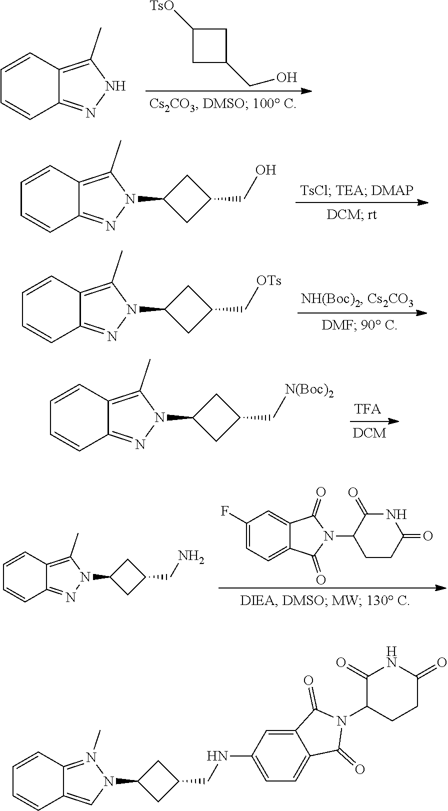

GS-688 was synthesized following the procedures for steps 2 to 6 of GS-684 to provide the title compound (75 mg, yield: 8% over 5 steps) as a yellow solid. MS (ESI) m/z=472.7 [M+H]⁺.

### Example 50: 2-(2,6-Dioxopiperidin-3-yl)-5-(((trans-3-(3-methyl-1H-indazol-1-yl)cyclobutyl)methyl)amino)isoindoline-1,3-dione (GS-689)

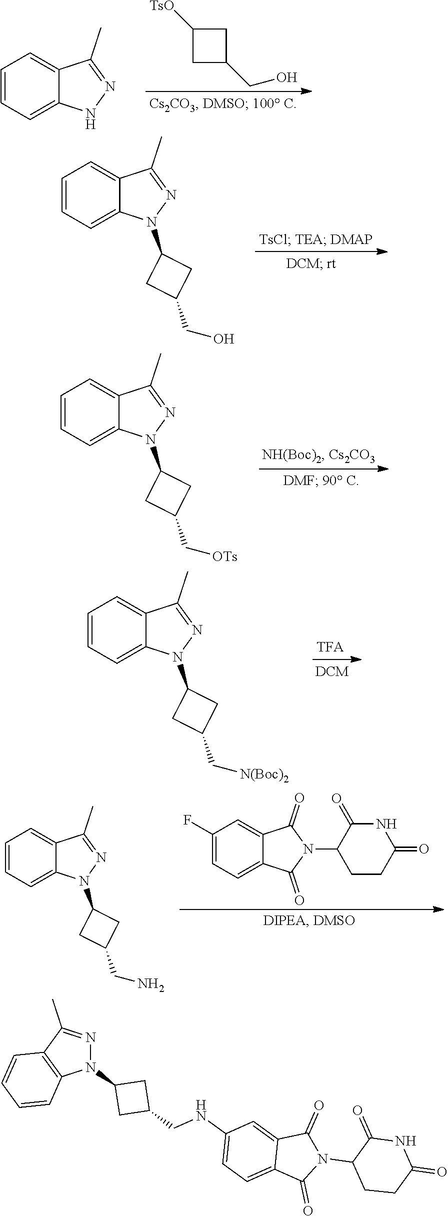

GS-689 was synthesized following the procedures for steps 2 to 6 of GS-684 to provide the title compound (87 mg, yield: 25% over 5 steps) as a yellow solid. MS (ESI) m/z=472.6 [M+H]⁺.

### Example 51: 5-(((Trans-3-(3′-cyclopropyl-2-methyl-1′H,2H-[3,4′-bipyrazol]-1′-yl)cyclobutyl)methyl)amino)-2-(2,6-dioxopiperidin-3-yl)isoindoline-1,3-dione (GS-690)

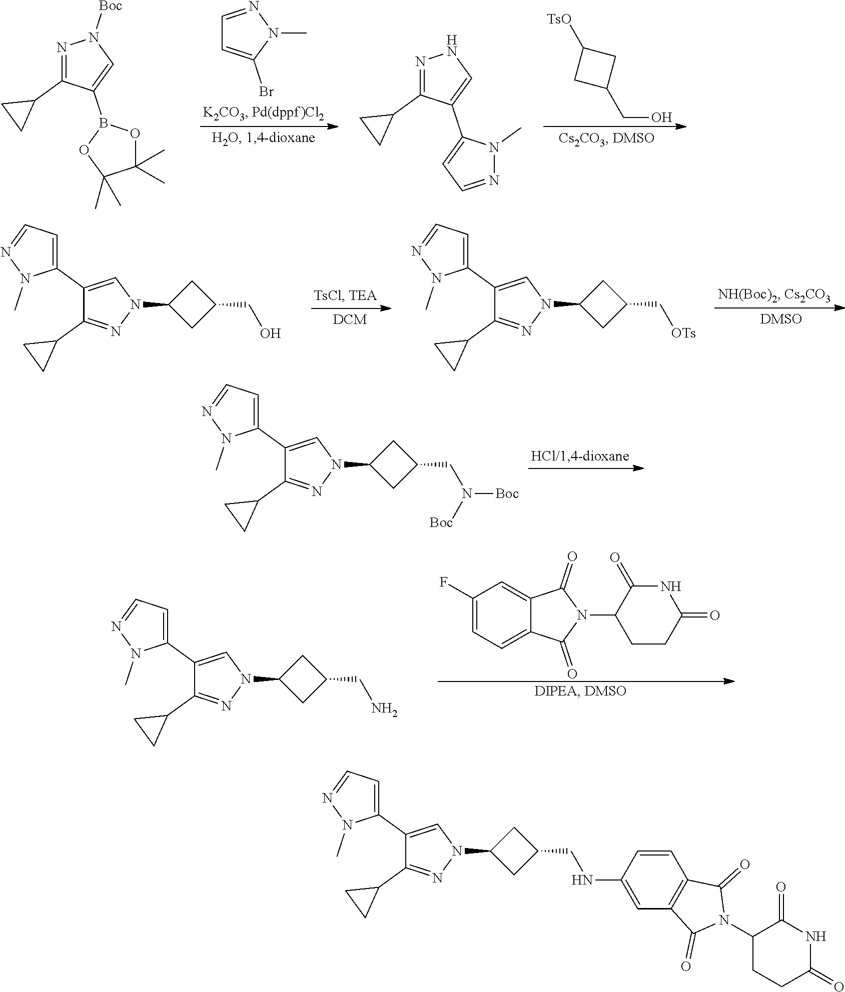

### Step 1. Synthesis of 3′-cyclopropyl-2-methyl-1′H,2H-3,4′-bipyrazole

To a mixture of tert-butyl 3-cyclopropyl-4-(4,4,5,5-tetramethyl-1,3,2-dioxaborolan-2-yl)pyrazole-1-carboxylate (600 mg, 1.80 mmol) and 5-bromo-1-methyl-pyrazole (289.03 mg, 1.80 mmol) in 1,4-dioxane (10 mL) were added K₂CO₃ (743.23 mg, 5.39 mmol), Pd(dppf)Cl₂ (131.36 mg, 179.52 μmol) and water (1 mL). The resulting mixture was degassed with N₂ and heated to 100 [symbol] for 16 h. Then the mixture was filtered, and the filtrate was concentrated in vacuo. The resulting residue was purified using a C18 column to provide the title compound (120 mg, yield: 35%) as a colorless oil. MS (ESI) m/z=189.4 [M+H]⁺.

The remaining steps were performed according to the procedures for steps 2 to 6 of GS-684 to provide the desired product (5 mg, yield: 1% over 5 steps) as a yellow solid. MS (ESI) m/z=528.7 [M+H]⁺.

### Example 52: 5-(((Trans-3-(3-cyclopropyl-4-(1-methyl-1H-imidazol-5-yl)-1H-pyrazol-1-yl)cyclobutyl)methyl)amino)-2-(2,6-dioxopiperidin-3-yl)isoindoline-1,3-dione (GS-691)

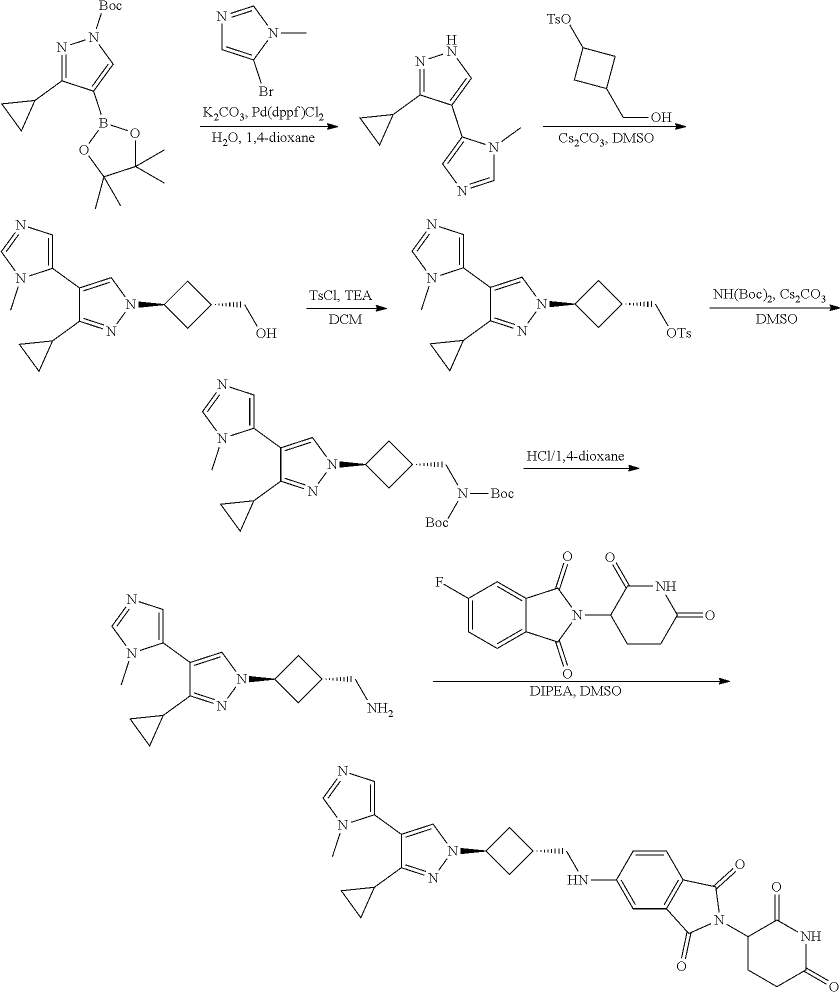

### Step 1. Synthesis of 3-cyclopropyl-4-(1-methyl-1H-imidazol-5-yl)-1H-pyrazole

To a mixture of tert-butyl 3-cyclopropyl-4-(4,4,5,5-tetramethyl-1,3,2-dioxaborolan-2-yl)pyrazole-1-carboxylate (1000 mg, 2.99 mmol) and 5-bromo-1-methyl-imidazole (481.72 mg, 2.99 mmol) in 1,4-dioxane (10 mL) were added K₂CO₃ (1.24 g, 8.98 mmol), Pd(dppf)Cl₂ (218.93 mg, 299.21 μmol) and water (1 mL). The resulting mixture was degassed with N₂ and heated to 100 [symbol] for 16 h. Then the mixture was filtered, and the filtrate was concentrated in vacuo. The resulting residue was purified using a C18 column to provide the title compound as a colorless oil. MS (ESI) m/z=189.4 [M+H]⁺.

The remaining steps were performed according to the procedures for steps 2 to 6 of GS-684 to provide the desired product (4 mg, yield: 10% over 5 steps) as a light yellow solid. MS (ESI) m/z=528.7 [M+H]⁺.

### Example 53: 2-(2,6-Dioxopiperidin-3-yl)-5-(((trans-3-(7-methyl-1H-indazol-1-yl)cyclobutyl)methyl)amino)isoindoline-1,3-dione (GS-693)

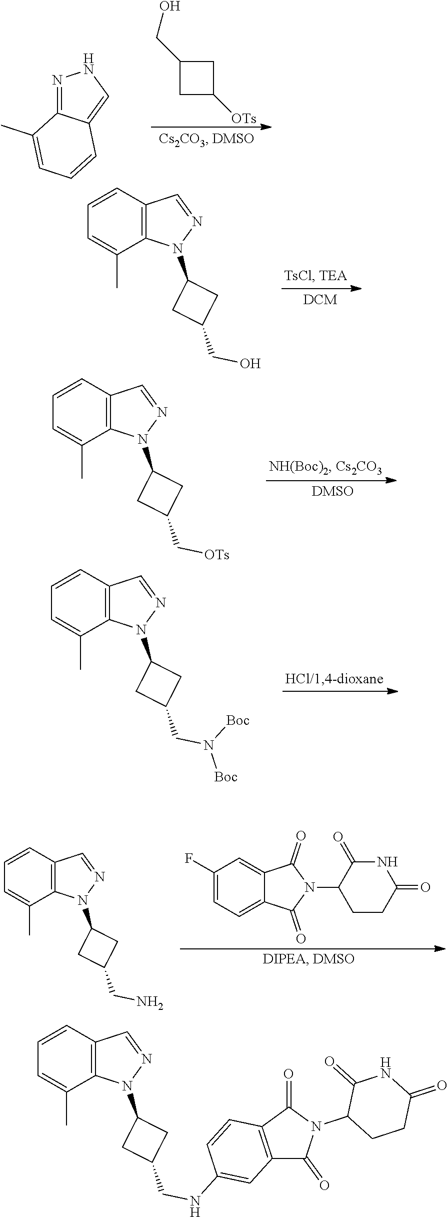

GS-693 was synthesized following the procedures of steps 2 to 6 of GS-684 to provide the title compound (12 mg, yield: 1% over 5 steps) as a light yellow solid. MS (ESI) m/z=472.6 [M+H]⁺.

### Example 54: 2-(2,6-Dioxopiperidin-3-yl)-5-(((trans-3-(7-methyl-2H-indazol-2-yl)cyclobutyl)methyl)amino)isoindoline-1,3-dione (GS-694)

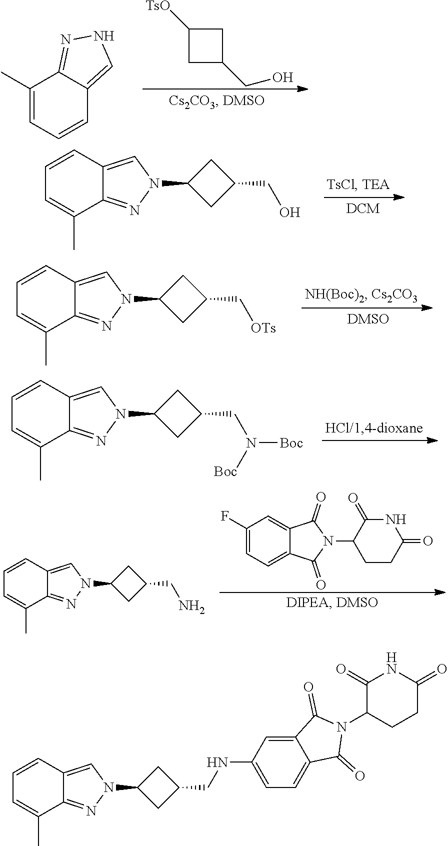

GS-694 was synthesized following the procedures of steps 2 to 6 of GS-684 to provide the title compound(15 mg, yield: 1% over 5 steps) as a light yellow solid. MS (ESI) m/z=472.6 [M+H]⁺.

### Example 55: 5-(((Trans-3-(3-cyclopropyl-4-(1-methyl-1H-pyrrolo[3,2-b]pyridin-5-yl)-1H-pyrazol-1-yl)cyclobutyl)methyl)amino)-2-(2,6-dioxopiperidin-3-yl)isoindoline-1,3-dione (GS-695)

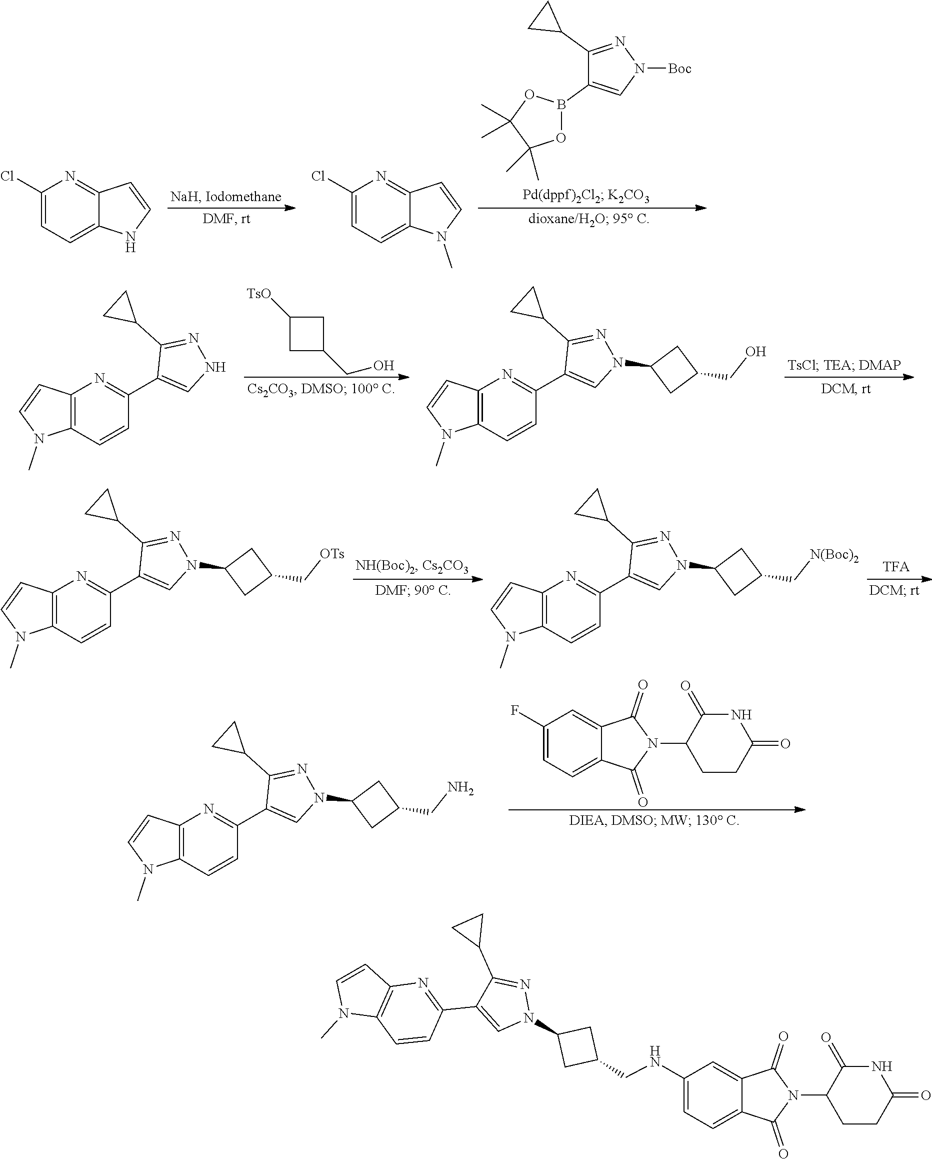

### Step 1. Synthesis of 5-chloro-1-methyl-1H-pyrrolo[3,2-b]pyridine

To a solution of 5-chloro-1H-pyrrolo[3,2-b]pyridine (850 mg, 5.57 mmol) in DMF (10 mL) was added NaH (60% in mineral oil, 446 mg, 11.14 mmol) at 0 [symbol] The mixture was stirred at rt for 15 min, before iodomethane (1.62 g, 11.4 mmol) was added dropwise. After the mixture was stirred for 30 min, it was poured into ice water and extracted with EtOAc twice. The organic layers were combined and washed with brine twice. The combined organic layers were dried, concentrated, and purified by silica gel flash chromatography to provide the title compound (877 mg, yield: 94%) as a white solid. MS (ESI) m/z=168.0 [M+H]⁺.

### Step 2. Synthesis of 5-(3-cyclopropyl-1H-pyrazol-4-yl)-1-methyl-1H-pyrrolo[3,2-b]pyridine

To a solution of 5-chloro-1-methyl-1H-pyrrolo[3,2-b]pyridine (250 mg, 1.50 mmol) in dioxane (7.5 mL) and water (2 mL) were added K₂CO₃ (1.02 g, 7.5 mmol), Pd(dppf)Cl₂ (110 mg, 0.15 mmol) and tert-butyl 3-cyclopropyl-4-(4,4,5,5-tetra methyl-1,3,2-dioxaborolan-2-yl)-1H-pyrazole-1-carboxylate (1.25 g, 3.74 mmol). The mixture was stirred at 90 [symbol] overnight under N₂. The mixture was purified by silica gel flash chromatography to provide the desired product (197 mg, yield: 55%) as a brown solid. MS (ESI) m/z=239.5 [M+H]⁺.

### Step 3. Synthesis of (trans-3-(3-cyclopropyl-4-(1-methyl-1H-pyrrolo[3,2-b]pyridin-5-yl)-1H-pyrazol-1-yl)cyclobutyl)methanol

To a solution of 5-(3-cyclopropyl-1H-pyrazol-4-yl)-1-methyl-1H-pyrrolo[3,2-b]pyridine (197 mg, 0.83 mmol) in DMSO (4 mL) were added Cs₂CO₃ (1.1 g, 3.32 mmol) and 3-(hydroxymethyl)cyclobutyl 4-methylbenzenesulfonate (427 mg, 1.66 mmol). The mixture was stirred at 100 [symbol] for 2.5 h under N₂. The mixture was poured into water and extracted with EtOAc (4×). The combined organic layers were washed with brine. The organic layer was dried, concentrated, and purified by silica gel flash chromatography to provide the desired trans-isomer (104 mg, yield: 30%) as a brown oil. MS (ESI) m/z=323.6 [M+H]⁺.

### Step 4. Synthesis of (trans-3-(3-cyclopropyl-4-(1-methyl-1H-pyrrolo[3,2-b]pyridin-5-yl)-1H-pyrazol-1-yl)cyclobutyl)methyl 4-methylbenzenesulfonate

To a solution of (trans-3-(3-cyclopropyl-4-(1-methyl-1H-pyrrolo[3,2-b]pyridin-5-yl)-1H-pyrazol-1-yl)cyclobutyl)methanol (104 mg, 0.32 mmol) in DCM (4 mL) were added DMAP (37 mg, 0.30 mmol), TEA (97 mg, 0.96 mmol) and 4-methylbenzenesulfonyl chloride (114 mg, 0.59 mmol). After the mixture was stirred at 25 [symbol] for 2 h, it was purified by silica gel flash chromatography to provide the title compound (85 mg, yield: 55%) as a brown solid. MS (ESI) m/z=477.6 [M+H]⁺.

### Step 5. Synthesis of tert-butyl N-tert-butoxycarbonyl-N-[[trans-3-[3-cyclopropyl-4-(1-methylpyrrolo[3,2-b]pyridin-5-yl)pyrazol-1-yl]cyclobutyl]methyl]carbamate

To a solution of (trans-3-(3-cyclopropyl-4-(1-methyl-1H-pyrrolo[3,2-b]pyridin-5-yl)-1H-pyrazol-1-yl)cyclobutyl)methyl 4-methylbenzenesulfonate (85 mg, 0.18 mmol) in DMSO (5 mL) and was added Cs₂CO₃ (117 mg, 0.36 mmol) and tert-but N-tert-butoxycarbonylcarbamate (58 mg, 0.27 μmol). The mixture was stirred at 90 [symbol] for 1.5 h under N₂. The mixture was purified by silica gel flash chromatography to provide the title compound (70 mg, yield: 75%) as a light-yellow solid. MS (ESI) m/z=522.8 [M+H]⁺.

### Step 6. Synthesis of (trans-3-(3-cyclopropyl-4-(5-methyl-5H-pyrrolo[3,2-d]pyrimidin-2-yl)-1H-pyrazol-1-yl)cyclobutyl)methanamine

A mixture of tert-butyl N-tert-butoxycarbonyl-N-[[trans-3-[3-cyclopropyl-4-(1-methylpyrrolo[3,2-b]pyridin-5-yl)pyrazol-1-yl]cyclobutyl]methyl]carbamate (70 mg, 0.13 mmol) and TFA (2 mL) in DCM (6 mL) was stirred at rt for 1 h. The solvents were removed to provide the title compound (40 mg, yield: 93%) as a white solid. MS (ESI) m/z=322.4 [M+H]⁺.

### Step 7. Synthesis of 5-(((trans-3-(3-cyclopropyl-4-(1-methyl-1H-pyrrolo[3,2-b]pyridin-5-yl)-1H-pyrazol-1-yl)cyclobutyl)methyl)amino)-2-(2,6-dioxopiperidin-3-yl)isoindoline-1,3-dione

A mixture of (trans-3-(3-cyclopropyl-4-(5-methyl-5H-pyrrolo[3,2-d]pyrimidin-2-yl)-1H-pyrazol-1-yl)cyclobutyl)methanamine (40 mg, 0.125 mmol), 2-(2,6-dioxo-3-piperidyl)-5-fluoro-isoindoline-1,3-dione (41 mg, 0.15 mmol) and DIPEA (323 mg, 2.5 mmol) in DMSO (4 mL) was heated to 130 [symbol] for 1.5 h under microwave irradiation. The mixture was purified by reverse phase column (CH₃CN/H₂O/TFA) and prep-TLC (DCM:MeOH=12:1) to provide the title compound (33 mg, yield: 46%) as a yellow solid. MS (ESI) m/z=578.8 [M+H]⁺.

### Example 56: 5-(((Trans-3-(3-cyclopropyl-4-(1-isopropyl-1H-pyrrolo[3,2-b]pyridin-5-yl)-1H-pyrazol-1-yl)cyclobutyl)methyl)amino)-2-(2,6-dioxopiperidin-3-yl)isoindoline-1,3-dione (GS-696)

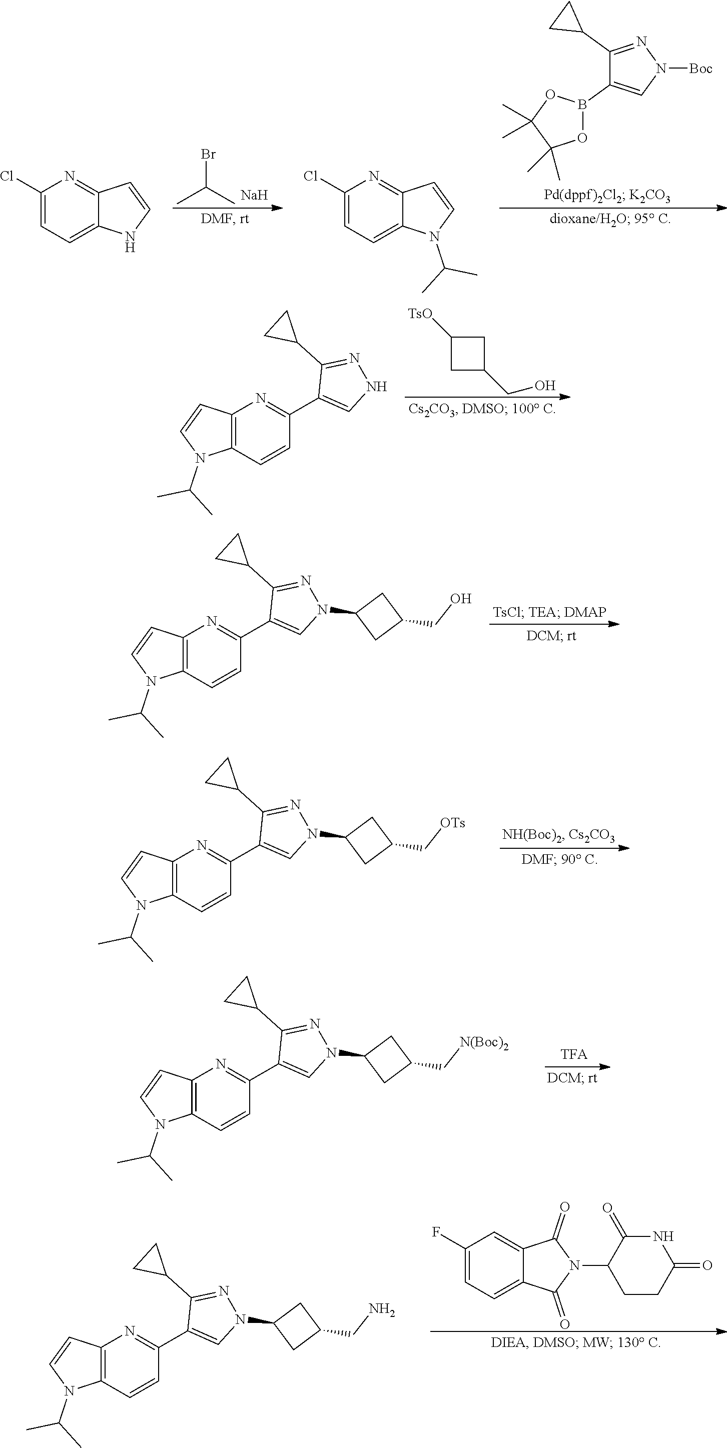

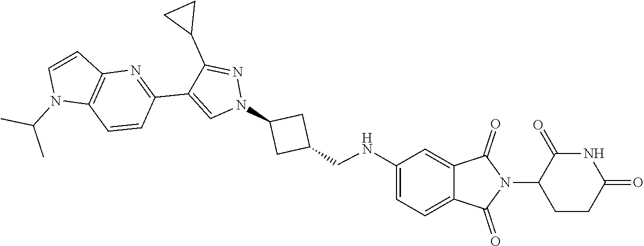 [SMILES: CC(C)n1ccc2nc(-c3cn([C@H]4C[C@H](CNc5ccc6c(c5)C(=O)N(C5CCC(=O)NC5=O)C6=O)C4)nc3C3CC3)ccc21]

### Step 1. Synthesis of 5-chloro-1-isopropyl-1H-pyrrolo[3.2-b]pyridine

To a solution of 5-chloro-1H-pyrrolo[3,2-b]pyridine (900 mg, 5.9 mmol) in DMF (10 mL) was added NaH (60% in mineral oil, 472 mg, 11.8 mmol) at 0 [symbol] . The mixture was stirred at rt for 15 min, before 2-bromopropane (1.45 g, 11.8 mmol) was added dropwise. After the resulting mixture was stirred at 50 [symbol] for 2 h, it was cooled to rt, poured into ice/water, and extracted with EtOAc. The organic layers were combined and washed with brine. The organic layer was dried, concentrated, and purified by silica gel flash chromatography to provide the desired product (1.0 g, yield: 87%) as a white solid. MS (ESI) m/z=196.1 [M+H]⁺.

### Step 2. Synthesis of 5-(3-cyclopropyl-1H-pyrazol-4-yl)-1-isopropyl-1H-pyrrolo[3,2-b]pyridine

To a solution of 5-chloro-1-isopropyl-1H-pyrrolo[3,2-b]pyridine (400 mg, 2.05 mmol) in dioxane (8 mL) and water (2 mL) were added K₂CO₃ (1.4 g, 10.25 mmol), Pd(dppf)Cl₂ (150 mg, 0.21 mmol) and tert-butyl 3-cyclopropyl-4-(4,4,5,5-tetramethyl-1,3,2-dioxaborolan-2-yl)-1H-pyrazole-1-carboxylate (1.7 g, 5.13 mmol). The mixture was stirred at 90 [symbol] overnight under N₂. The mixture was purified by silica gel flash chromatography to give the title compound (197 mg, yield: 26%) as a brown solid. MS (ESI) m/z=267.5 [M+H]⁺.

The remaining steps were performed according to the procedures for steps 2 to 6 of GS-684 to provide the desired product (3.2 mg, yield: 1% over 5 steps) as a yellow solid. MS (ESI) m/z=606.7 [M+H]⁺.

### Example 57: 5-(((Trans-3-(3-cyclopropyl-4-(5-fluoro-1,7-naphthyridin-8-yl)-1H-pyrazol-1-yl)cyclobutyl)methyl)amino)-2-(2,6-dioxopiperidin-3-yl)isoindoline-1,3-dione (GS-697)

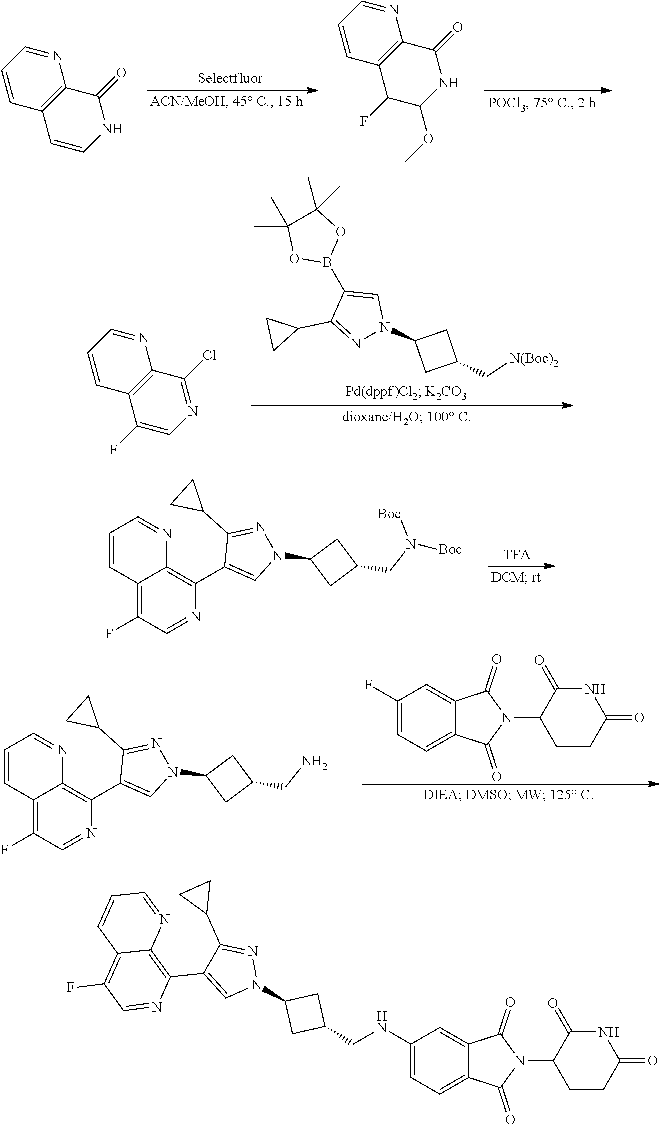

### Step 1. Synthesis of 5-fluoro-6-methoxy-6,7-dihydro-1,7-naphthyridin-8 (5H)-one

A solution of 1,7-naphthyridin-8 (7H)-one (5.0 g, 34.2 mmol) and selectfluor (12.1 g, 34.2 mmol) in acetonitrile (50 mL)/MeOH (10 mL) was stirred at 45 [symbol] for 15 h. After cooled to rt, the mixture was diluted with water (200 mL) and extracted with EtOAc (100 mL×3). The combined organic phase was washed with brine, dried over Na₂SO₄, filtered and concentrated in vacuo to give a residue, which was purified by silica gel chromatography (DCM:MeOH=10:1) to provide the title compound (2.0 g, yield: 30%) as a white solid. MS (ESI) m/z=197.1 [M+H]⁺.

### Step 2. Synthesis of 8-chloro-5-fluoro-1,7-naphthyridine

A solution of 5-fluoro-6-methoxy-6,7-dihydro-1,7-naphthyridin-8 (5H)-one (2.0 g, 10.2 mmol) in POCl₃ (20 mL) was stirred at 75 [symbol] for 2 h. After cooled to rt, the mixture was quenched with water (100 mL) and basified with aq. NaHCO₃ solution. The mixture was extracted with EtOAc (50 mL×3). The combined organic phase was washed with brine, dried over Na₂SO₄, filtered and concentrated in vacuo to give a residue, which was purified by prep-HPLC (0.10% TFA) to provide the title compound (669 mg, yield: 36%) as a white solid. ¹H NMR (400 MHz, DMSO-d₆) δ 9.25 (dd, J=8.0, 1.2 Hz, 1H), 8.66 ((dd, J=8.4, 1.2 Hz, 1H), 8.52 (s, 1H), 8.03 ((dd, J=8.8, 4.4 Hz, 1H). MS (ESI) m/z=182.6 [M+H]⁺.

### Step 3. Synthesis of tert-butyl (tert-butoxycarbonyl)((trans-3-(3-cyclopropyl-4-(5-fluoro-1,7-naphthyridin-8-yl)-1H-pyrazol-1-yl)cyclobutyl)methyl)carbamate

To a stirred solution of 8-chloro-5-fluoro-1,7-naphthyridine (100.0 mg, 547.7 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (425.1 mg, 821.5 μmol) in 1,4-dioxane (10 mL) and H₂O (1 mL) were added Pd(dppf)Cl₂ (40.0 mg, 54.7 μmol) and K₂CO₃ (188.9 mg, 1.37 mmol). The mixture was stirred at 130 [symbol] overnight under Ar, before it was diluted with H₂O (50 mL). The mixture was extracted with EtOAc (50 mL×3). The combined organic layers were washed with brine, dried over Na₂SO₄ and concentrated. The residue was purified by silica gel chromatography to provide the title compound (32.2 mg, yield: 11%) as a solid. MS (ESI) m/z=538.5 [M+H]⁺.

### Step 4. Synthesis of (trans-3-(3-cyclopropyl-4-(5-fluoro-1,7-naphthyridin-8-yl)-1H-pyrazol-1-yl)cyclobutyl)methanamine

To a stirred solution of tert-butyl (tert-butoxycarbonyl)((trans-3-(3-cyclopropyl-4-(5-fluoro-1,7-naphthyridin-8-yl)-1H-pyrazol-1-yl)cyclobutyl)methyl)carbamate (32.0 mg, 59.5 μmol) in DCM (10 mL) was added TFA (2.5 mL). After the mixture was stirred at rt for 1.5 h, it was concentrated to provide the title compound (20.0 mg, yield: 99%). MS (ESI) m/z=338.5 [M+H]⁺.

### Step 5. Synthesis of 5-(((trans-3-(3-cyclopropyl-4-(5-fluoro-1,7-naphthyridin-8-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(5-fluoro-1,7-naphthyridin-8-yl)-1H-pyrazol-1-yl)cyclobutyl)methanamine (20 mg, 59.28 μmol) in DMSO (2.5 mL) were added 2-(2,6-dioxopiperidin-3-yl)-5-fluoroisoindoline-1,3-dione (21.2 mg, 77.1 μmol) and DIEA (76.4 mg, 592.7 μmol). The mixture was stirred at 130 [symbol] for 2.5 h under microwave irradiation, before it was purified by reverse phase chromatography to provide the title compound (5.0 mg, yield: 14%) as a yellow solid. MS (ESI) m/z=594.5 [M+H]⁺.

### Example 58: 5-(((Trans-3-(3-cyclopropyl-4-(1-methyl-1H-pyrazolo[4,3-b]pyridin-5-yl)-1H-pyrazol-1-yl)cyclobutyl)methyl)amino)-2-(2,6-dioxopiperidin-3-yl)isoindoline-1,3-dione (GS-699)

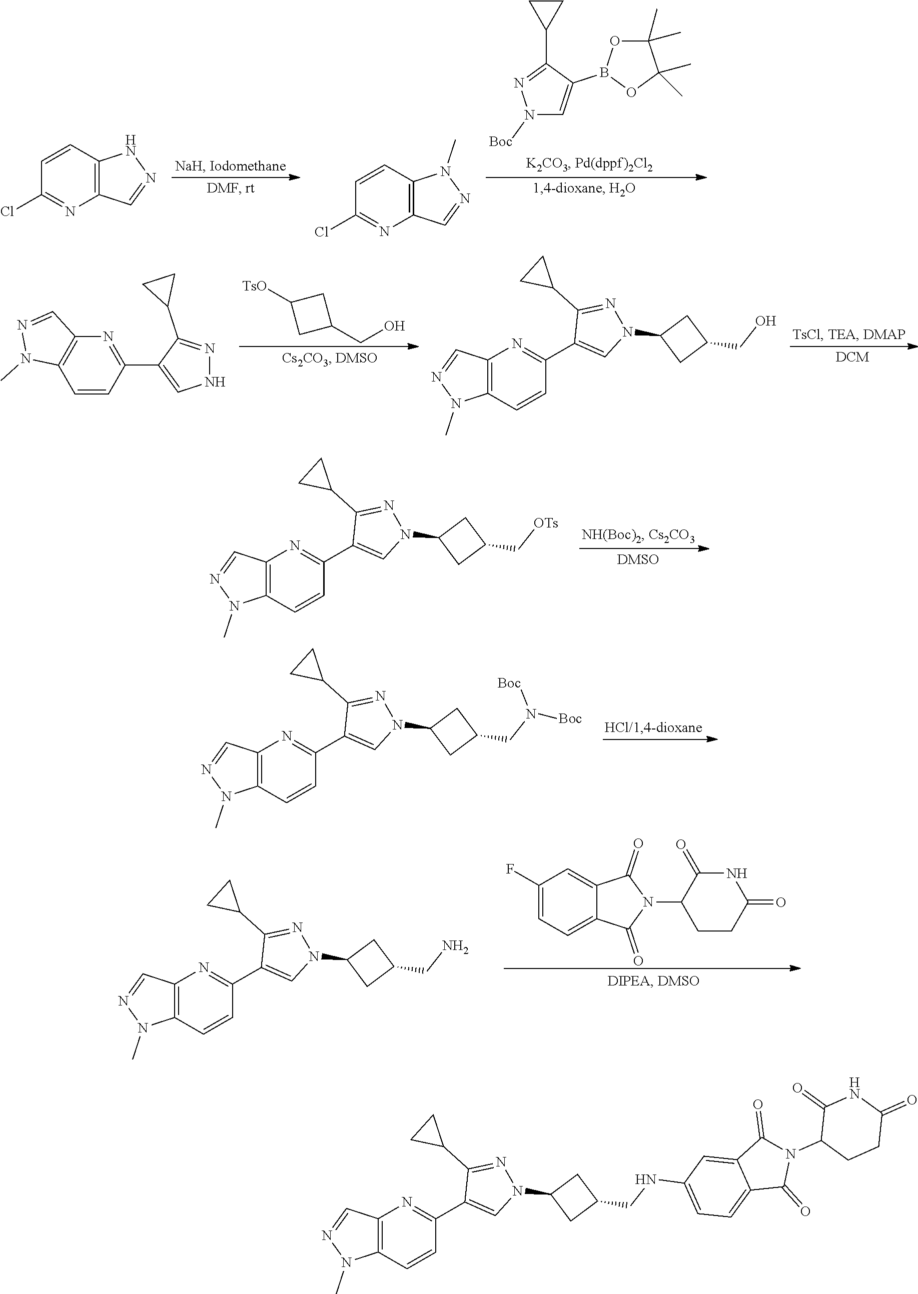

### Step 1. Synthesis of 5-chloro-1-methyl-1H-pyrazolo[4,3-b]pyridine

To a mixture of 5-chloro-1H-pyrazolo[4,3-b]pyridine (1 g, 6.51 mmol) in DMF (10 mL) was added NaH (60% in mineral oil, 312.56 mg, 13.02 mmol). The resulting mixture was stirred at 25 [symbol] for 15 min, before iodomethane (1.11 g, 7.81 mmol) was added. After the mixture was stirred for 1 h, it was quenched with ice/water, and extracted with EtOAc (30 mL×3). The combined organics were washed with brine (30 mL×2), dried over Na₂SO₄ and concentrated in vacuo. The residue was purified by silica gel chromatography to provide the title compound (600 mg, yield: 55%) as a white solid. MS (ESI) m/z=168.3 [M+H]⁺.

### Step 2. Synthesis of 5-(3-cyclopropyl-1H-pyrazol-4-yl)-1-methyl-1H-pyrazolo[4,3-b]pyridine

To a mixture of 5-chloro-1-methyl-pyrazolo[4,3-b]pyridine (600 mg, 3.58 mmol) and tert-butyl 3-cyclopropyl-4-(4,4,5,5-tetramethyl-1,3,2-dioxaborolan-2-yl)pyrazole-1-carboxylate (1.79 g, 5.37 mmol) in H₂O (1 mL) and 1,4-dioxane (5 mL) were added K₂CO₃ (988.09 mg, 7.16 mmol) and Pd(dppf)Cl₂ (261.95 mg, 358.01 μmol). The resulting mixture was heated to 100 [symbol] and stirred under N₂ atmosphere for 2 h. After cooled to rt, the mixture was treated with water, and extracted with EtOAc (30 mL×3). The combined organic layers were washed with brine (30 mL×2), dried over Na₂SO₄, filtered and concentrated in vacuo. The residue was purified by silica gel chromatography to provide the title compound (300 mg, yield: 35%) as a light yellow solid. MS (ESI) m/z=240.4 [M+H]⁺.

The remaining steps were performed according to the procedures for steps 2 to 6 of GS-684 to provide the desired product (13 mg, yield: 3% over 5 steps) as a yellow solid. MS (ESI) m/z=579.7 [M+H]⁺.

### Example 59: 5-(((Trans-3-(3-cyclopropyl-4-(1-isopropyl-1H-pyrazolo[4,3-b]pyridin-5-yl)-1H-pyrazol-1-yl)cyclobutyl)methyl)amino)-2-(2,6-dioxopiperidin-3-yl)isoindoline-1,3-dione (GS-700)

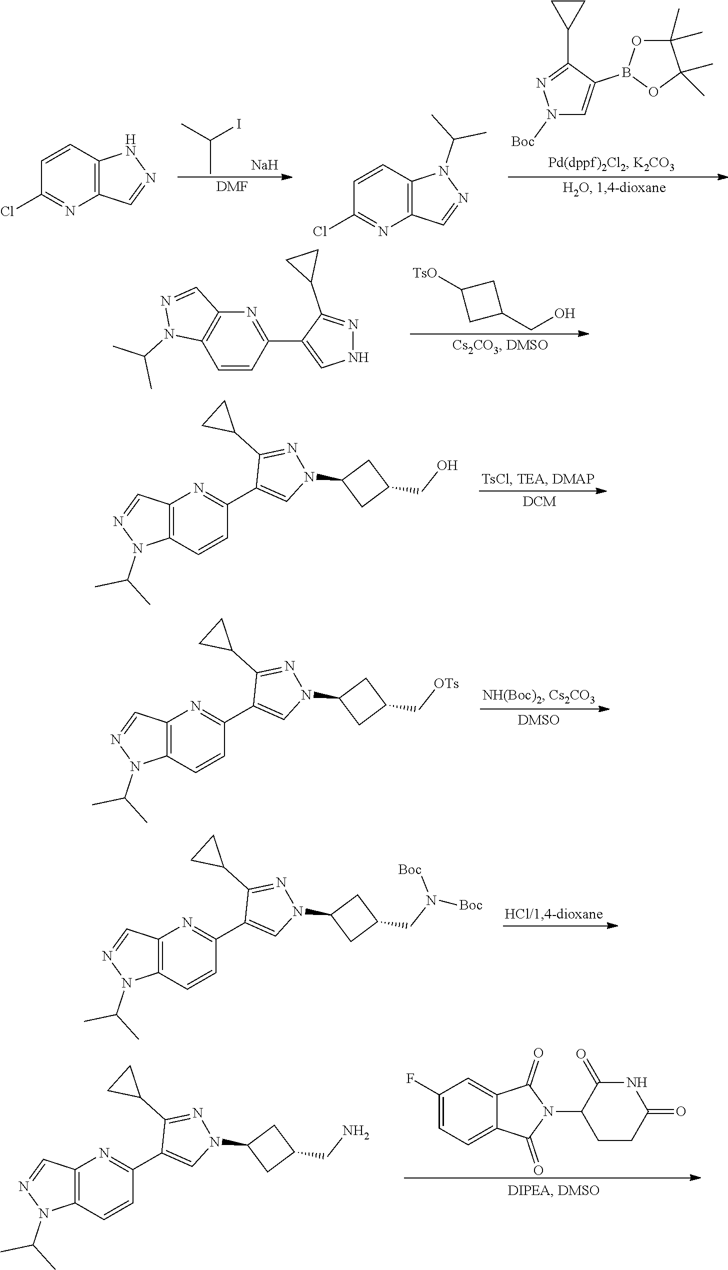

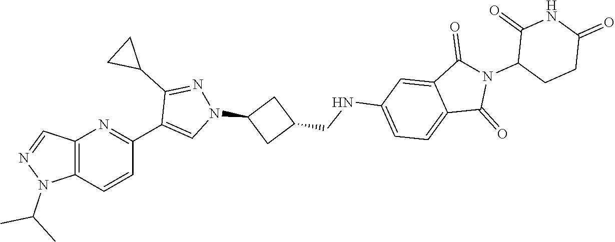 [SMILES: CC(C)n1ncc2nc(-c3cn([C@H]4C[C@H](CNc5ccc6c(c5)C(=O)N(C5CCC(=O)NC5=O)C6=O)C4)nc3C3CC3)ccc21]

### Step 1. Synthesis of 5-chloro-1-isopropyl-1H-pyrazolo[4,3-b]pyridine

To a mixture of 5-chloro-1H-pyrazolo[4,3-b]pyridine (1 g, 6.51 mmol) in DMF (10 mL) was added NaH (600% in mineral oil, 312.56 mg, 13.02 mmol). The resulting mixture was stirred at 25 [symbol] for 15 min, before 2-bromopropane (961.06 mg, 7.81 mmol) was added. After the reaction was stirred for 1 h, it was quenched with ice, and extracted with EtOAc (30 mL×3). The combined organic layers were washed with brine (30 mL×2), dried over Na₂SO₄, concentrated in vacuo. The resulting residue was purified by a silica gel column to provide the title compound (500 mg, yield: 39%) as a white solid. MS (ESI) m/z=196.3 [M+H]⁺.

### Step 2. Synthesis of 5-(3-cyclopropyl-1H-pyrazol-4-yl)-1-isopropyl-1H-pyrazolo[4,3-b]pyridine

To a mixture of 5-chloro-1-isopropyl-pyrazolo[4,3-b]pyridine (500 mg, 2.56 mmol) and tert-butyl 3-cyclopropyl-4-(4,4,5,5-tetramethyl-1,3,2-dioxaborolan-2-yl)pyrazole-1-carboxylate (1.28 g, 3.83 mmol) in H₂O (1 mL) and 1,4-dioxane (5 mL) were added K₂CO₃ (705.35 mg, 5.11 mmol) and Pd(dppf)Cl₂ (186.99 mg, 255.56 μmol). The resulting mixture was stirred at 100 [symbol] under N₂ atmosphere for 2 h. The mixture was cooled to rt, diluted with water, and extracted with EtOAc (30 mL×3). The combined organic layers were washed with brine (30 mL×2), dried over Na₂SO₄, and concentrated. The resulting residue was purified by silica gel chromatography to provide the title compound (300 mg, yield: 44%) as a light yellow solid. MS (ESI) m/z=268.4 [M+H]⁺.

The remaining steps were performed according to the procedures for steps 2 to 6 of GS-684 to provide the desired product (21 mg, yield: 4% over 5 steps) as a yellow solid. MS (ESI) m/z=607.8 [M+H]⁺.

### Example 60: 5-(((Trans-3-(3-cyclopropyl-4-(4-hydroxycyclohexyl)-1H-pyrazol-1-yl)cyclobutyl)methyl)amino)-2-(2,6-dioxopiperidin-3-yl)isoindoline-1,3-dione (GS-701)

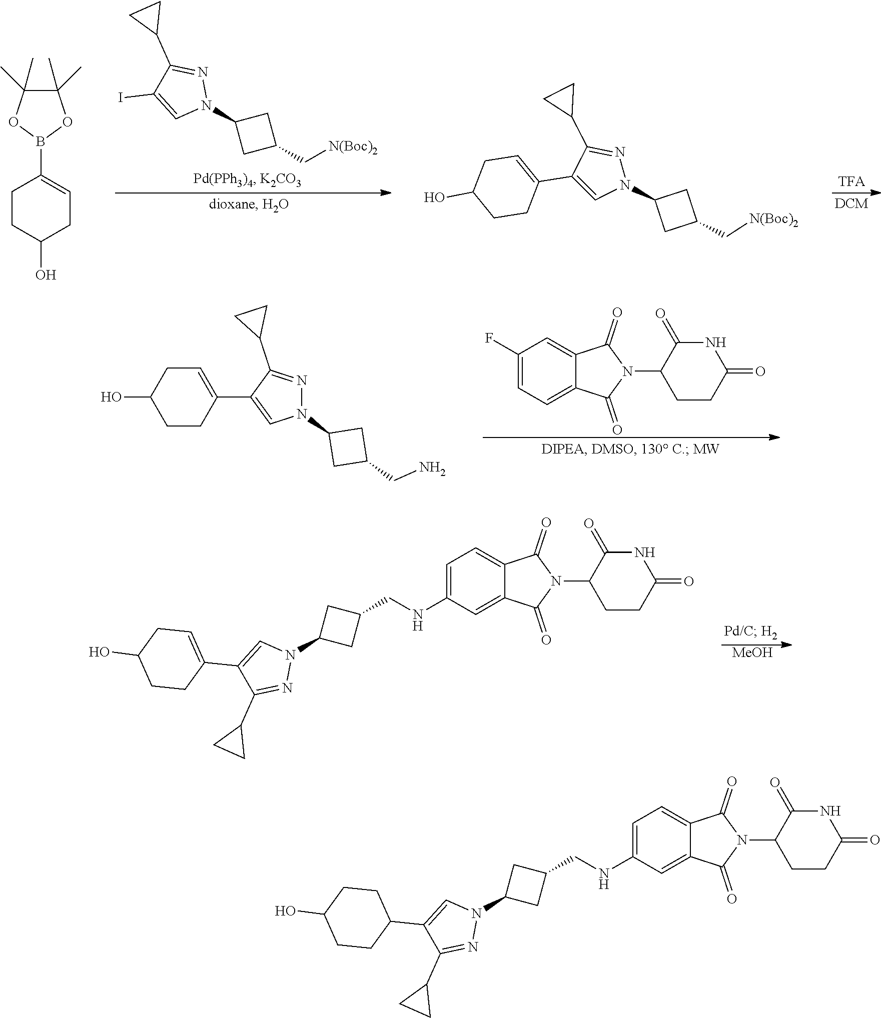

### Step 1. Synthesis of tert-butyl N-tert-butoxycarbonyl-N-[[trans-3-[3-cyclopropyl-4-(4-hydroxycyclohexen-1-yl)pyrazol-1-yl]cyclobutyl]methyl]carbamate

To a solution of 4-(4,4,5,5-tetramethyl-1,3,2-dioxaborolan-2-yl)cyclohex-3-en-1-ol (280 mg, 1.25 mmol) in dioxane (10 mL) and water (2.5 mL) were added Pd(PPh₃)₄ (144.43 mg, 124.94 μmol), K₂CO₃ (517.26 mg, 3.75 mmol) and tert-butyl N-tert-butoxycarbonyl-N-[[3-(3-cyclopropyl-4-iodo-pyrazol-1-yl)cyclobutyl]methyl]carbamate (840.38 mg, 1.62 mmol). The mixture was stirred at 100 [symbol] for 12 h under N₂. The mixture was purified by silica gel chromatography (petroleum ether:EtOAc=1:0 to 3:1) to provide the title compound (200 mg, yield: 33%) as a brown oil. MS (ESI) m/z=488.9 [M+H]⁺.

Step 2 to step 3 were performed according to the procedures for steps 5 to 6 of GS-684 to provide the desired product (80 mg, yield: 35% over 2 steps) as a yellow solid. MS (ESI) m/z=544.6 [M+H]⁺.

### Step 4. Synthesis of 5-(((trans-3-(3-cyclopropyl-4-(4-hydroxycyclohexyl)-1H-pyrazol-1-yl)cyclobutyl)methyl)amino)-2-(2.6-dioxopiperidin-3-yl)isoindoline-1,3-dione

To a solution of 5-(((trans-3-(3-cyclopropyl-4-(4-hydroxycyclohex-1-en-1-yl)-1H-pyrazol-1-yl)cyclobutyl)methyl)amino)-2-(2,6-dioxopiperidin-3-yl)isoindoline-1,3-dione (80 mg, 147.16 μmol) in MeOH (5 mL) was added Pd/C (50 mg). The reaction mixture was stirred at rt for 2 h under H₂. The reaction solution was filtered, concentrated, and purified by prep-TLC (DCM:MeOH=15:1) to provide the desired product (45 mg, yield: 56%) as a yellow solid. MS (ESI) m/z=546.7 [M+H]⁺.

### Example 61: 5-(((Trans-3-(3-cyclopropyl-4-(5-((2,3-dihydroxypropyl)amino)quinoxalin-2-yl)-1H-pyrazol-1-yl)cyclobutyl)methyl)amino)-2-(2,6-dioxopiperidin-3-yl)isoindoline-1,3-dione (GS-702)

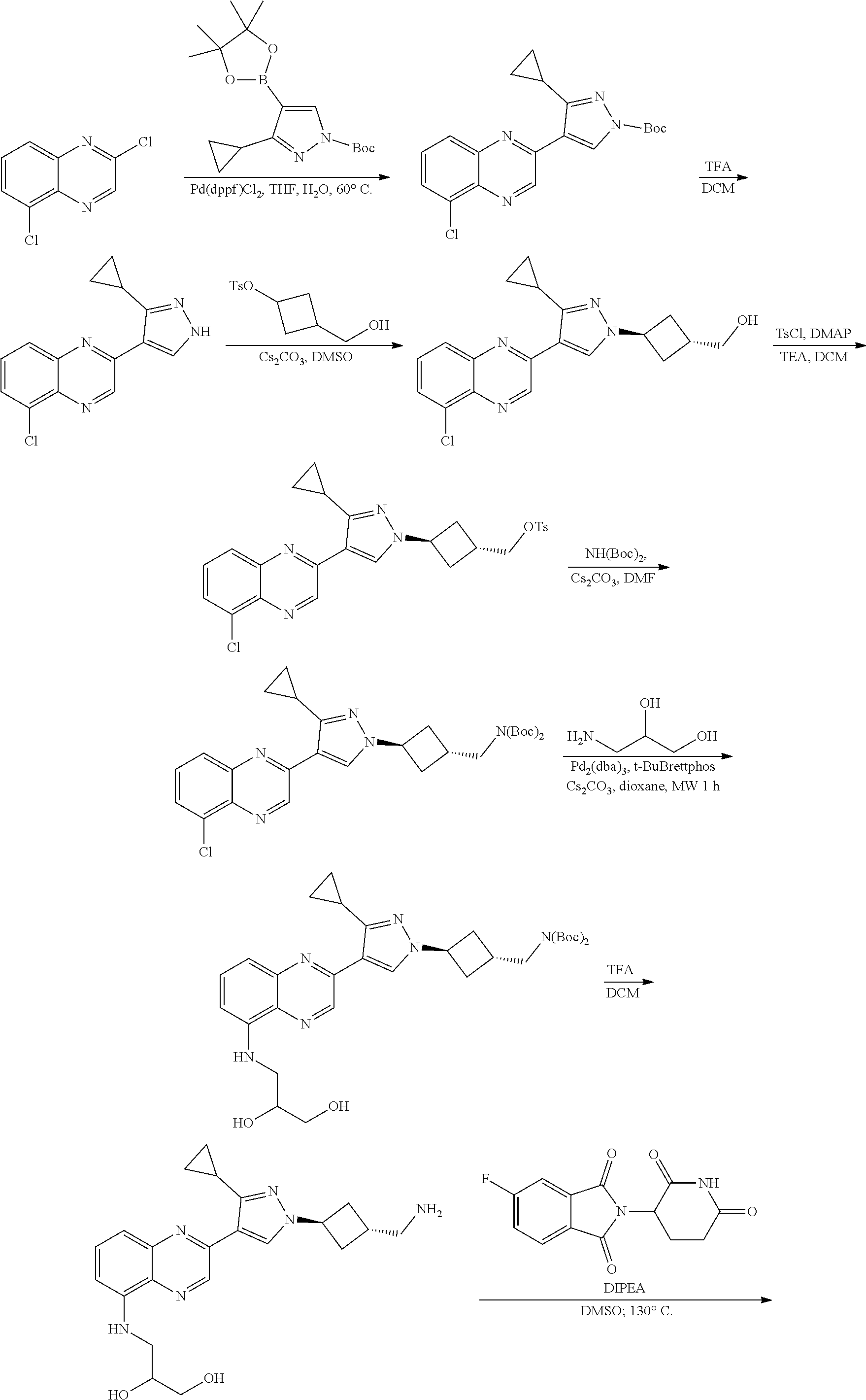

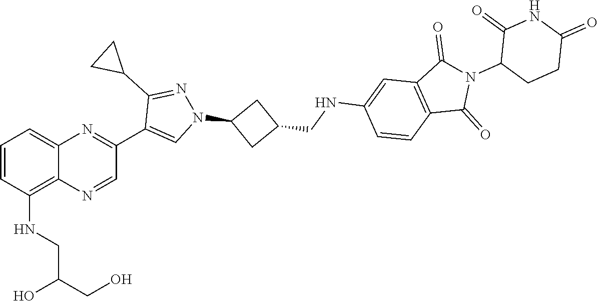 [SMILES: O=C1CCC(N2C(=O)c3ccc(NC[C@H]4C[C@H](n5cc(-c6cnc7c(NCC(O)CO)cccc7n6)c(C6CC6)n5)C4)cc3C2=O)C(=O)N1]

### Step 1. Synthesis of tert-butyl 4-(5-chloroquinoxalin-2-yl)-3-cyclopropyl-1H-pyrazole-1-carboxylate

To a solution of 2,5-dichloroquinoxaline (10 g, 50.24 mmol) and tert-butyl 3-cyclopropyl-4-(4,4,5,5-tetramethyl-1,3,2-dioxaborolan-2-yl)-1H-pyrazole-1-carboxylate (20.15 g, 60.29 mmol) in dioxane (200 mL) and water (50 mL) were added Pd(dppf)Cl₂ (3.67 g, 5.02 mmol) and K₂CO₃ (20.80 g, 150.73 mmol) at rt under N₂. After the mixture was heated to 100° C. for 2 h, it was cooled to rt, and extracted with EtOAc (100 mL). The organic solvent was concentrated and the resulting residue was purified by silica gel chromatography (petroleum ether:EtOAc=10:1 to 5:1) to provide the title compound (14 g, yield: 75%). MS (ESI) m/z=371.5 [M+H]⁺

### Step 2. Synthesis of 5-chloro-2-(3-cyclopropyl-1H-pyrazol-4-yl)quinoxaline

To a solution of tert-butyl 4-(5-chloroquinoxalin-2-yl)-3-cyclopropyl-1H-pyrazole-1-carboxylate (1.24 g, 3.34 mmol) in DCM (10 mL) was added TFA (2 mL) at rt. After the mixture was stirred at rt for 1 h, it was concentrated. The residue was diluted with NaHCO₃ solution (200 mL) and extracted with EtOAc (200 mL×3). The organic solvents were concentrated and the resulting residue was purified by silica gel chromatography (petroleum ether:EtOAc=10:1 to 0:1) to provide the title compound (890 mg, yield: 98%) as a yellow solid. MS (ESI) m/z=271.4 [M+H]⁺.

### Step 3. Synthesis of (trans-3-(4-(5-chloroquinoxalin-2-yl)-3-cyclopropyl-1H-pyrazol-1-yl)cyclobutyl)methanol

To a solution of 5-chloro-2-(3-cyclopropyl-1H-pyrazol-4-yl)quinoxaline (0.9 g, 3.32 mmol) and 3-(hydroxymethyl)cyclobutyl 4-methylbenzenesulfonate (1.28 g, 4.99 mmol) in DMF (20 mL) was added Cs₂CO₃ (3.24 g, 9.97 mmol) at rt. After the mixture was stirred at 90° C. for 3 h, it was cooled to rt, quenched with brine (50 mL), and extracted with EtOAc (50 mL×3). The combined organic layers were washed with brine (100 mL×2), dried over Na₂SO₄, and concentrated. The resulting residue was purified by silica gel chromatography (petroleum ether:EtOAc=1:1 to 0:1) to provide the desired trans-isomer (1.0 g, yield: 85%). MS (ESI) m/z=355.5 [M+H]⁺.

### Step 4. Synthesis of (trans-3-(4-(5-chloroquinoxalin-2-yl)-3-cyclopropyl-1H-pyrazol-1-yl)cyclobutyl)methyl 4-methylbenzenesulfonate

To a solution of (trans-3-(4-(5-chloroquinoxalin-2-yl)-3-cyclopropyl-1H-pyrazol-1-yl)cyclobutyl)methanol (1.0 g, 2.82 mmol) in TEA (10 mL) and DCM (20 mL) was added DMAP (344.30 mg, 2.82 mmol) and 4-methylbenzenesulfonyl chloride (1.07 g, 5.64 mmol) at 0° C. The mixture was warmed to rt and stirred for 2 h. Then the mixture was concentrated and the resulting residue was purified by silica gel chromatography (petroleum ether:EtOAc=10:1 to 3:1) to provide the title compound (1.2 g, yield: 84%) as a yellow solid. MS (ESI) m/z=509.5 [M+H]⁺.

### Step 5. Synthesis of trans-tert-butyl N-tert-butoxycarbonyl-N-[[3-[4-(5-chloroquinoxalin-2-yl)-3-cyclopropyl-pyrazol-1-yl]cyclobutyl]methyl]carbamate

To a solution of (trans-3-(4-(5-chloroquinoxalin-2-yl)-3-cyclopropyl-1H-pyrazol-1-yl)cyclobutyl)methyl 4-methylbenzenesulfonate (1.02 g, 4.71 mmol) and Cs₂CO₃ (2.30 g, 7.07 mmol) was added tert-butyl N-tert-butoxycarbonylcarbamate (1.12 g, 5.17 mmol) at rt. After the mixture was stirred at 90° C. for 3 h, it was cooled to rt, and concentrated. The resulting residue was purified by silica gel chromatography (petroleum ether:EtOAc=10:1 to 5:1) to provide the title compound (1.2 g, yield: 92%) as a yellow solid. MS (ESI) m/z=554.7 [M+H]⁺.

### Step 6. Synthesis of tert-butyl ((trans-3-(3-cyclopropyl-4-(5-((2,3-dihydroxypropyl)amino)quinoxalin-2-yl)-1H-pyrazol-1-yl)cyclobutyl)methyl)carbamate

To a solution of trans-tert-butyl N-tert-butoxycarbonyl-N-[[3-[4-(5-chloroquinoxalin-2-yl)-3-cyclopropyl-pyrazol-1-yl]cyclobutyl]methyl]carbamate (65 mg, 117.31 μmol) and 3-aminopropane-1,2-diol (213.76 mg, 2.35 mmol) in dioxane (1 mL) were added Pd₂(dba)₃ (10.74 mg, 11.73 μmol), Cs₂CO₃ (114.67 mg, 351.94 μmol) and t-BuBrettphos (11.37 mg, 23.46 μmol) at rt. The mixture was stirred at 120 [symbol] for 1 h under microwave irradiation. The mixture was purified by reversed phase chromatography to provide the title compound (35 mg, yield: 58%) as a white solid. MS (ESI) m/z=508.7 [M+H]⁺.

### Step 7. Synthesis of 3-((2-(1-(trans-3-(aminomethyl)cyclobutyl)-3-cyclopropyl-1H-pyrazol-4-yl)quinoxalin-5-yl)amino)propane-1,2-diol

To a solution of tert-butyl ((trans-3-(3-cyclopropyl-4-(5-((2,3-dihydroxypropyl)amino)quinoxalin-2-yl)-1H-pyrazol-1-yl)cyclobutyl)methyl)carbamate (35 mg, 68.81 μmol) in DCM (0.5 mL) was added TFA (78.46 mg, 688.15 μmol). After the mixture was stirred at 20 [symbol] for 1 h, it was concentrated to provide the title compound (35.96 mg, yield: 100%) as a white solid. MS (ESI) m/z=408.5 [M+H]⁺.

### Step 8. Synthesis of 5-(((trans-3-(3-cyclopropyl-4-(5-((2,3-dihydroxypropyl)amino)quinoxalin-2-yl)-1-pyrazol-1-yl)cyclobutyl)methyl)amino)-2-(2,6-dioxopiperidin-3-yl)isoindoline-1,3-dione

A mixture of 3-((2-(1-(trans-3-(aminomethyl)cyclobutyl)-3-cyclopropyl-1H-pyrazol-4-yl)quinoxalin-5-yl)amino)propane-1,2-diol (30 mg, 57.41 μmol), 2-(2,6-dioxo-3-piperidyl)-5-fluoro-isoindoline-1,3-dione (23.79 mg, 86.12 μmol) and DIEA (73.49 mg, 574.14 μmol) in DMSO (1 mL) was heated at 130 [symbol] for 2 h under microwave irradiation. The resulting mixture was purified by reverse phase chromatography to provide the title compound (10 mg, yield: 26%) as a yellow solid. MS (ESI) m/z=665.5 [M+H]⁺.

### Example 62: 5-(((Trans-3-(3-cyclopropyl-4-(3-hydroxycyclohexyl)-1H-pyrazol-1-yl)cyclobutyl)methyl)amino)-2-(2,6-dioxopiperidin-3-yl)isoindoline-1,3-dione (GS-703)

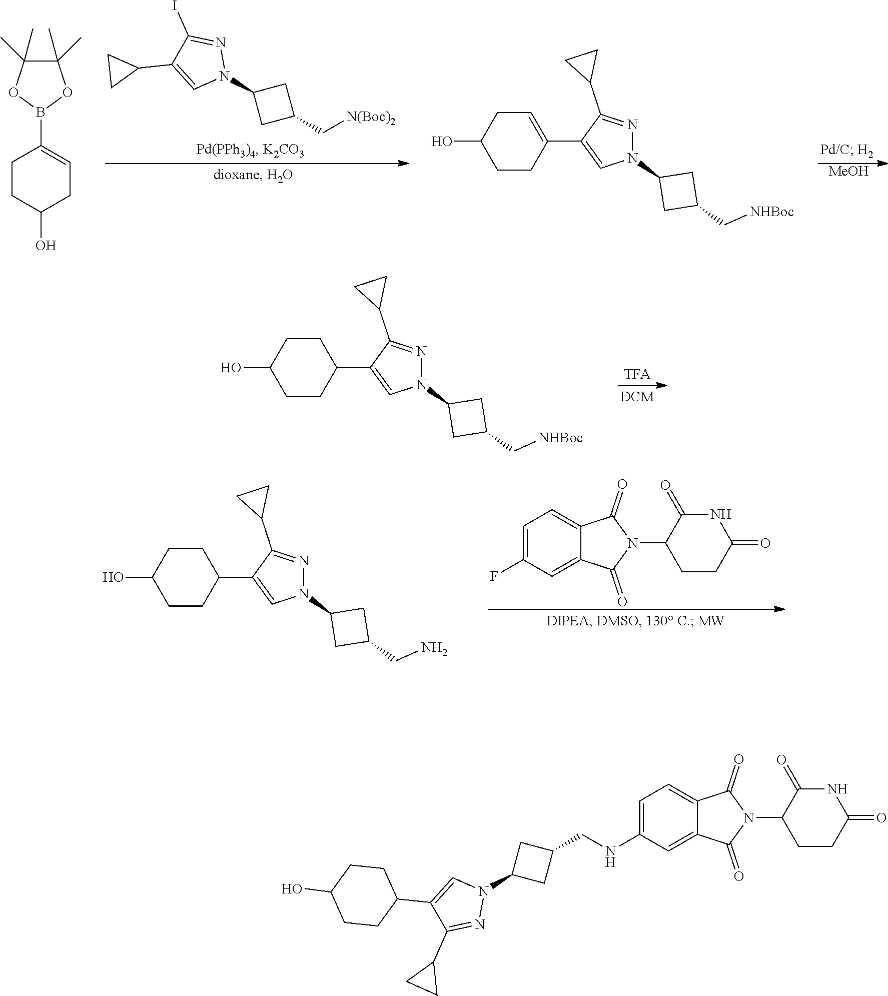

GS-703 was synthesized according to the procedures for steps 3 to 6 of GS-646 to provide the title compound (65 mg, yield: 29% over 4 steps) as a yellow solid. MS (ESI) m/z=546.8 [M+H]⁺.

### Example 63: 5-(((Trans-3-(3-cyclopropyl-4-(1-(2,2,2-trifluoroethyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-704)

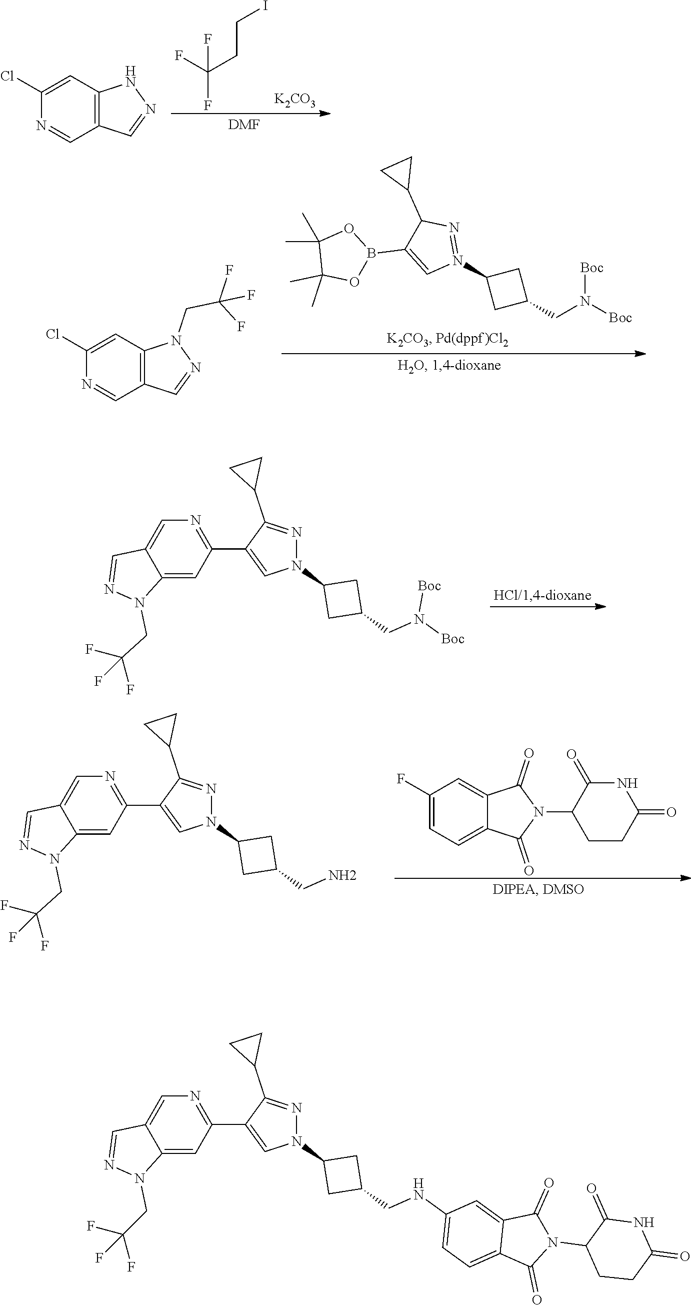

### Step 1. Synthesis of 6-chloro-1-(2,2,2-trifluoroethyl)-1H-pyrazolo[4.3-c]pyridine

To a mixture of 6-chloro-1H-pyrazolo[4,3-c]pyridine (1 g, 6.51 mmol) in DMF (10 mL) were added K₂CO₃ (1.80 g, 13.02 mmol) and 1,1,1,-trifluoro-2-iodo-ethane (1.64 g, 7.81 mmol). The resulting mixture was stirred at 60 [symbol] for 16 h. The mixture was diluted with water and extracted with EtOAc (40 mL×3) The combined organic layers were washed with brine (30 mL×2), dried over Na₂SO₄, and concentrated in vacuo. The resulting residue was purified by silica gel chromatography to provide the title compound (500 mg, yield: 32%) as a white solid. MS (ESI) m/z=236.4 [M+H]⁺.

The remaining steps were performed according to the procedures for steps 2 to 4 of GS-641 to provide the desired product (3 mg, yield: 2% over 3 steps) as a yellow solid. MS (ESI) m/z=647.6 [M+H]⁺.

### Example 64: 5-(((Trans-3-(3-cyclopropyl-4-(5-hydroxy-6,7-dihydro-5H-cyclopenta[b]pyridin-2-yl)-1H-pyrazol-1-yl)cyclobutyl)methyl)amino)-2-(2,6-dioxopiperidin-3-yl)isoindoline-1,3-dione (GS-705)

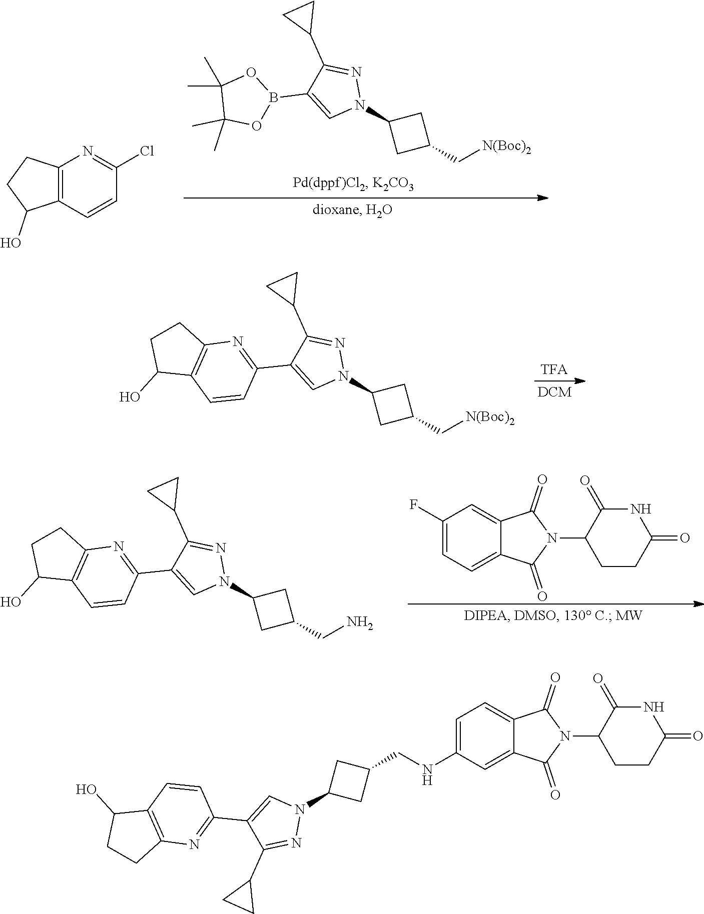

GS-705 was synthesized according to the procedures for steps 2 to 4 of GS-641 to provide the title compound (20 mg, yield: 3% over 3 steps) as a yellow solid. MS (ESI) m/z=581.7 [M+H]⁺.

### Example 65: 5-(((Trans-3-(4-(7-(3-aminopyrrolidin-1-yl)quinoxalin-2-yl)-3-cyclopropyl-1H-pyrazol-1-yl)cyclobutyl)methyl)amino)-2-(2,6-dioxopiperidin-3-yl)isoindoline-1,3-dione (GS-706)

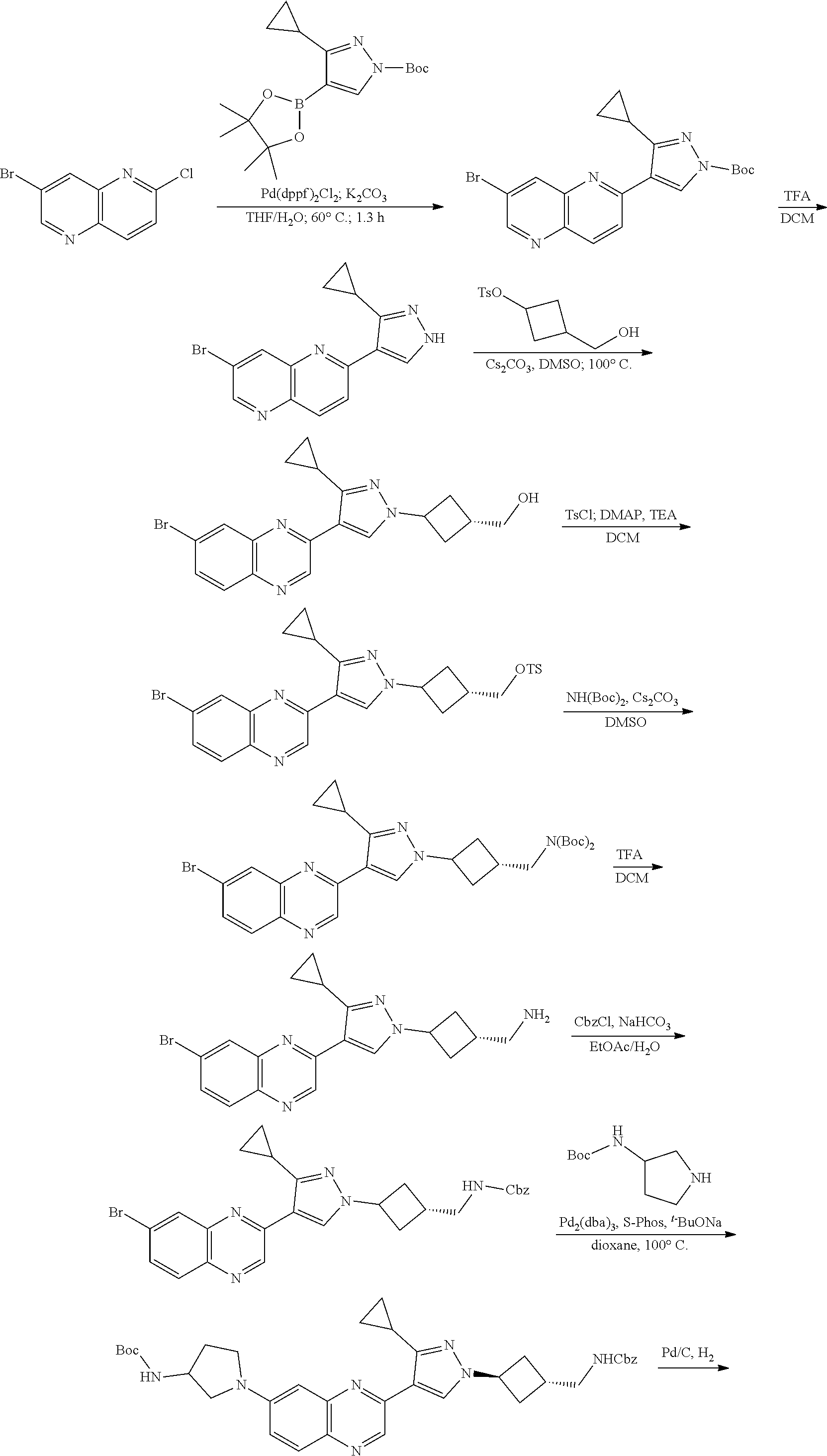

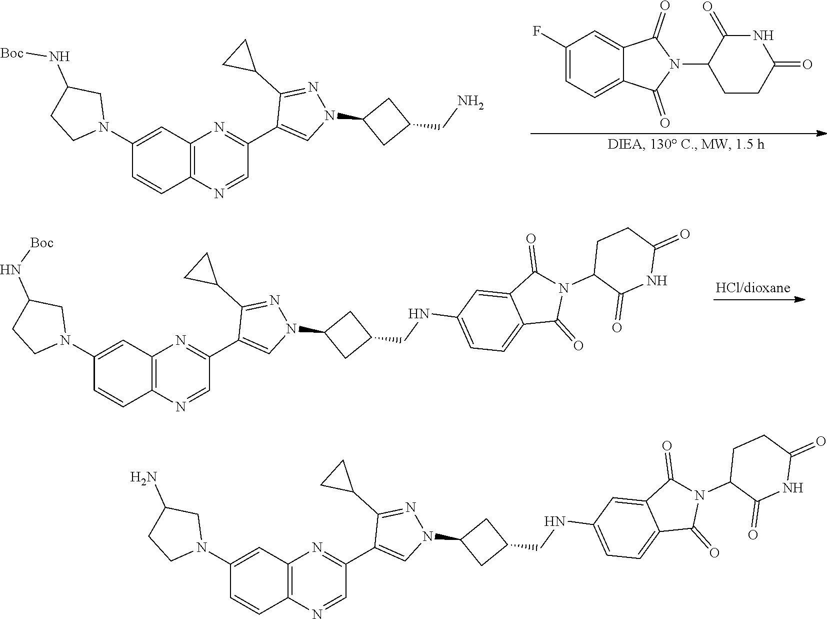 [SMILES: CC(C)(C)OC(=O)NC1CCN(c2ccc3ncc(-c4cn([C@H]5C[C@H](CN)C5)nc4C4CC4)nc3c2)C1 ; O=C1CCC(N2C(=O)c3ccc(F)cc3C2=O)C(=O)N1 ; CC(C)(C)OC(=O)NC1CCN(c2ccc3ncc(-c4cn([C@H]5C[C@H](CNc6ccc7c(c6)C(=O)N(C6CCC(=O)NC6=O)C7=O)C5)nc4C4CC4)nc3c2)C1 ; NC1CCN(c2ccc3ncc(-c4cn([C@H]5C[C@H](CNc6ccc7c(c6)C(=O)N(C6CCC(=O)NC6=O)C7=O)C5)nc4C4CC4)nc3c2)C1]

### Step 1. Synthesis of tert-butyl 4-(7-bromoquinoxalin-2-yl)-3-cyclopropyl-1H-pyrazole-1-carboxylate

To a solution of 7-bromo-2-chloro-1,5-naphthyridine (2.0 g, 8.21 mmol) in THF (25 mL) and water (7.5 mL) were added K₂CO₃ (2.83 g, 20.53 mmol), Pd(dppf)Cl₂ (600.44 mg, 821.40 μmol) and tert-butyl 3-cyclopropyl-4-(4,4,5,5-tetramethyl-1,3,2-dioxaborolan-2-yl)-1H-pyrazole-1-carboxylate (2.75 g, 8.21 mmol). The mixture was stirred at 60 [symbol] for 1.5 h under N₂. The mixture was purified by silica gel chromatography (petroleum ether:EtOAc=6:1) to provide the title compound (815 mg, yield: 24%) as a black oil. MS (ESI) m/z=415.2 [M+H]⁺.

### Step 2. Synthesis of 7-bromo-2-(3-cyclopropyl-1H-pyrazol-4-yl)quinoxaline

A mixture of tert-butyl 4-(7-bromoquinoxalin-2-yl)-3-cyclopropyl-1H-pyrazole-1-carboxylate (815 mg, 1.96 mmol) and TFA (5 mL) in DCM (10 mL) was stirred at rt for 1 h. Solvents were removed in vacuo to provide the title compound (700 mg, yield: 83%) as a black solid. MS (ESI) m/z=315.1 [M+H]⁺.

Step 3 to Step 5 were performed according to the procedures for steps 2 to 4 of GS-684 to provide the title compound (300 mg, yield: 10% over 3 steps) as a light-yellow solid. MS (ESI) m/z=598.3 [M+H]⁺.

### Step 6. Synthesis of (trans-3-(4-(7-bromoquinoxalin-2-yl)-3-cyclopropyl-1H-pyrazol-1-yl)cyclobutyl)methanamine

A mixture of tert-butyl N-[[trans-3-[4-(7-bromoquinoxalin-2-yl)-3-cyclopropyl-pyrazol-1-yl]cyclobutyl]methyl]-N-tert-butoxycarbonyl-carbamate (402 mg, 671.65 μmol) and TFA (3 mL) in DCM (9 mL) was stirred at rt for 1 h. Solvents were removed to provide the title compound (300 mg, yield: 87%) as a white solid. MS (ESI) m/z=398.3 [M+H]⁺.

### Step 7. Synthesis of benzyl ((trans-3-(4-(7-bromoquinoxalin-2-yl)-3-cyclopropyl-1H-pyrazol-1-yl)cyclobutyl)methyl)carbamate

To a solution of (trans-3-(4-(7-bromoquinoxalin-2-yl)-3-cyclopropyl-1H-pyrazol-1-yl)cyclobutyl)methanamine (300 mg, 753.20 μmol) and NaHCO₃ (316.35 mg, 3.77 mmol) in EtOAc (10 mL) and water (2 mL) was added CbzCl (193.20 mg, 1.13 mmol) under ice-bath. The mixture was stirred at rt for 1 h and purified by silica gel chromatography to provide the title compound (309 mg, yield: 77%) as a white solid. MS (ESI) m/z=532.3 [M+H]⁺.

### Step 8. Synthesis of benzyl ((trans-3-(4-(7-(3-((tert-butoxycarbonyl)amino)pyrrolidin-1-yl)quinoxalin-2-yl)-3-cyclopropyl-1H-pyrazol-1-yl)cyclobutyl)methyl)carbamate

To a solution of benzyl ((trans-3-(4-(7-bromoquinoxalin-2-yl)-3-cyclopropyl-1H-pyrazol-1-yl)cyclobutyl)methyl)carbamate (309 mg, 580.36 μmol) and tert-butyl N-pyrrolidin-3-ylcarbamate (216.18 mg, 1.16 mmol) in dioxane (15 mL) were added Cs₂CO₃ (378.39 mg, 1.16 mmol) and SPhos Pd G3 (100.52 mg, 116.07 μmol). After the mixture was stirred at 100 [symbol] for 2 h under N₂, it was purified by silica gel chromatography (petroleum ether:EtOAc=1:0 to 1:1) to provide the title compound (338 mg, yield: 910%) as a yellow oil. MS (ESI) m/z=638.7 [M+H]⁺.

### Step 9. Synthesis of tert-butyl (1-(3-(1-(trans-3-(aminomethyl)cyclobutyl)-3-cyclopropyl-1H-pyrazol-4-yl)quinoxalin-6-yl)pyrrolidin-3-yl)carbamate

To a solution of benzyl ((trans-3-(4-(7-(3-((tert-butoxycarbonyl)amino)pyrrolidin-1-yl)quinoxalin-2-yl)-3-cyclopropyl-1H-pyrazol-1-yl)cyclobutyl)methyl)carbamate (338 mg, 529.97 μmol) in MeOH (20 mL) was added Pd/C (100 mg). The reaction mixture was stirred under H₂ at rt for 6 h. The reaction mixture was filtered, concentrated to provide the title compound (240 mg, yield: 90%) as a yellow solid. MS (ESI) m/z=504.5 [M+H]⁺.

### Step 10. Synthesis of tert-butyl (1-(3-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)quinoxalin-6-yl)pyrrolidin-3-yl)carbamate

A mixture of tert-butyl (1-(3-(1-(trans-3-(aminomethyl)cyclobutyl)-3-cyclopropyl-1H-pyrazol-4-yl)quinoxalin-6-yl)pyrrolidin-3-yl)carbamate (182 mg, 361.37 μmol), 2-(2,6-dioxo-3-piperidyl)-5-fluoro-isoindoline-1,3-dione (131.63 mg, 476.53 μmol) and DIPEA (467.03 mg, 3.61 mmol) in DMSO (7 mL) was heated at 130 [symbol] for 1.5 h under microwave irradiation. The mixture was purified by reverse phase column (CH₃CN/H₂O/TFA) and prep-TLC (DCM:MeOH=18:1) to provide the title compound (80 mg, yield: 29%) as a yellow solid. MS (ESI) m/z=760.6 [M+H]⁺.

### Step 11. Synthesis of 5-(((trans-3-(4-(7-(3-aminopyrrolidin-1-yl)quinoxalin-2-yl)-3-cyclopropyl-1H-pyrazol-1-yl)cyclobutyl)methyl)amino)-2-(2,6-dioxopiperidin-3-yl)isoindoline-1,3-dione

A mixture of tert-butyl (1-(3-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)quinoxalin-6-yl)pyrrolidin-3-yl)carbamate (80 mg, 105.28 μmol) and HCl solution (4 M in dioxane, 6 mL) in dioxane (2 mL) was stirred at rt for 6 h. The solvents were removed, and the resulting residue was freeze-dried to provide the title compound (70 mg, yield: 95%) as a red solid. MS (ESI) m/z=660.6 [M+H]⁺.

### Example 66: 5-(((Trans-3-(3-cyclopropyl-4-(6-(dimethylamino)-5-fluoropyridin-2-yl)-1H-pyrazol-1-yl)cyclobutyl)methyl)amino)-2-(2,6-dioxopiperidin-3-yl)isoindoline-1,3-dione (GS-707)

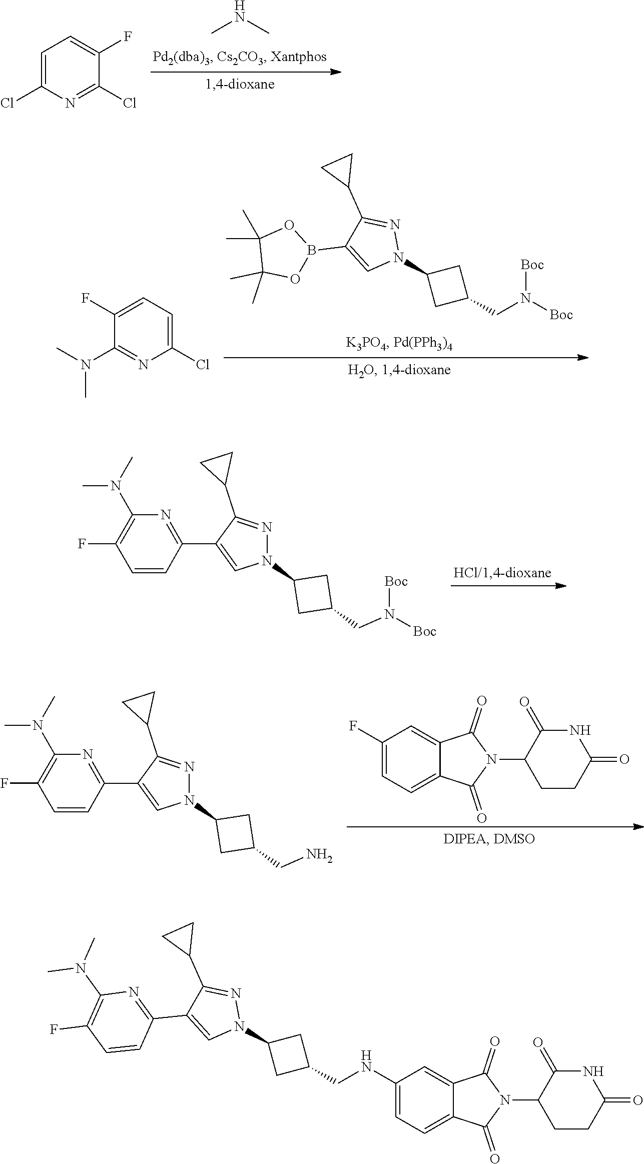

### Step 1. Synthesis of 6-chloro-3-fluoro-N,N-dimethylpyridin-2-amine

To a mixture of 2,6-dichloro-3-fluoro-pyridine (600 mg, 3.61 mmol) in 1,4-dioxane (5 mL) were added dimethylamine (2 M in THF, 3.6 mL, 7.23 mmol), xantphos (83.67 mg, 144.60 μmol), Cs₂CO₃ (2.36 g, 7.23 mmol) and Pd₂(dba)₃ (66.20 mg, 72.30 μmol). The resulting mixture was stirred at 100 [symbol] for 1 h in a sealed tube. The mixture was cooled to rt and poured into water. Then the mixture was extracted with EtOAc (30 mL×2). The combined organic layers were washed with brine (15 mL×2), dried over Na₂SO₄, and concentrated in vacuo. The resulting residue was purified by silica gel chromatography to provide the title compound (300 mg, yield: 47%) as a white solid. MS (ESI) m/z=175.1 [M+H]⁺.

The remaining steps were performed according to the procedures for steps 2 to 4 GS-641 to provide the desired product (5 mg, yield: 3% over 3 steps) as a yellow solid. MS (ESI) m/z=586.6 [M+H]⁺.

### Example 67: 5-(((Trans-3-(3-cyclopropyl-4-((tetrahydro-2H-pyran-4-yl)amino)-1H-pyrazol-1-yl)cyclobutyl)methyl)amino)-2-(2,6-dioxopiperidin-3-yl)isoindoline-1,3-dione (GS-708)

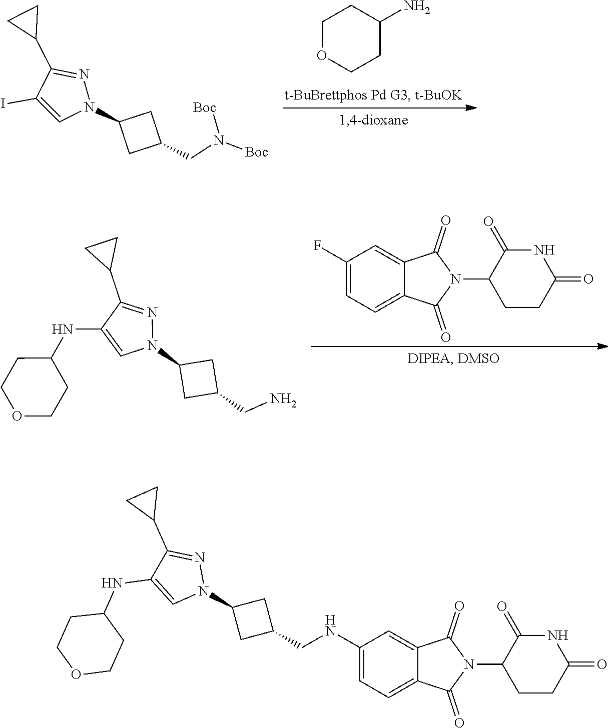 [SMILES: CC(C)(C)OC(=O)N(C[C@H]1C[C@H](n2cc(I)c(C3CC3)n2)C1)C(=O)OC(C)(C)C ; NC1CCOCC1 ; NC[C@H]1C[C@H](n2cc(NC3CCOCC3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(NC6CCOCC6)c(C6CC6)n5)C4)cc3C2=O)C(=O)N1]

### Step 1. Synthesis of 1-(trans-3-(aminomethyl)cyclobutyl)-3-cyclopropyl-N-(tetrahydro-2H-pyran-4-yl)-1H-pyrazol-4-amine

To a mixture of tert-butyl N-tert-butoxycarbonyl-N-[[trans-3-(3-cyclopropyl-4-iodo-pyrazol-1-yl)cyclobutyl]methyl]carbamate (500 mg, 966.25 μmol) and tetrahydropyran-4-amine (146.60 mg, 1.45 mmol) in 1,4-dioxane (5 mL) were added t-BuOK (325.27 mg, 2.90 mmol) and t-BuBrettphos G3 Pd (45.87 mg, 48.31 μmol). The resulting mixture was stirred at 100 [symbol] under N₂ atmosphere for 2 h. The mixture was cooled to rt and purified by reverse phase chromatography to provide the title compound (100 mg, yield: 36%) as a brown solid. MS (ESI) m/z=291.3 [M+H]⁺.

### Step 2. Synthesis of 5-(((trans-3-(3-cyclopropyl-4-((tetrahydro-2H-pyran-4-yl)amino)-1H-pyrazol-1-yl)cyclobutyl)methyl)amino)-2-(2,6-dioxopiperidin-3-yl)isoindoline-1,3-dione

To a mixture of 1-(trans-3-(aminomethyl)cyclobutyl)-3-cyclopropyl-N-(tetrahydro-2H-pyran-4-yl)-1H-pyrazol-4-amine (20 mg, 61.19 μmol) and 2-(2,6-dioxopiperidin-3-yl)-5-fluoroisoindoline-1,3-dione (25.35 mg, 91.78 μmol) in DMSO (2 mL) was added DIPEA (79.08 mg, 611.88 μmol, 101 μL).

The resulting mixture was heated at 130 [symbol] for 1.5 h under microwave irradiation. The mixture was purified by prep-TLC to provide the title compound (3.87 mg, yield: 12%) as a yellow solid. MS (ESI) m/z=547.6 [M+H]⁺.

### Example 68: 5-(((Trans-3-(3-cyclopropyl-4-((4-hydroxycyclohexyl)amino)-1H-pyrazol-1-yl)cyclobutyl)methyl)amino)-2-(2,6-dioxopiperidin-3-yl)isoindoline-1,3-dione (GS-709)

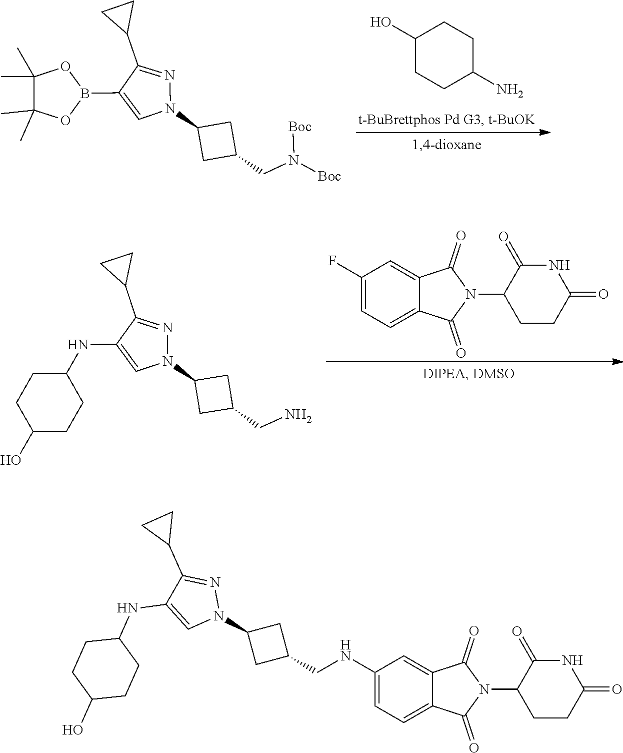 [SMILES: CC(C)(C)OC(=O)N(C[C@H]1C[C@H](n2cc(B3OC(C)(C)C(C)(C)O3)c(C3CC3)n2)C1)C(=O)OC(C)(C)C ; NC1CCC(O)CC1 ; NC[C@H]1C[C@H](n2cc(NC3CCC(O)CC3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(NC6CCC(O)CC6)c(C6CC6)n5)C4)cc3C2=O)C(=O)N1]

GS-709 was synthesized according to the procedures for steps 1 to 2 of GS-708 to provide the title compound (3.0 mg, yield: 2% over 2 steps) as a yellow solid. MS (ESI) m/z=560.7 [M+H]⁺.

### Example 69: 5-(((Trans-3-(3-cyclopropyl-4-(5-((1,3-dihydroxypropan-2-yl)amino)quinoxalin-2-yl)-1H-pyrazol-1-yl)cyclobutyl)methyl)amino)-2-(2,6-dioxopiperidin-3-yl)isoindoline-1,3-dione (GS-710)

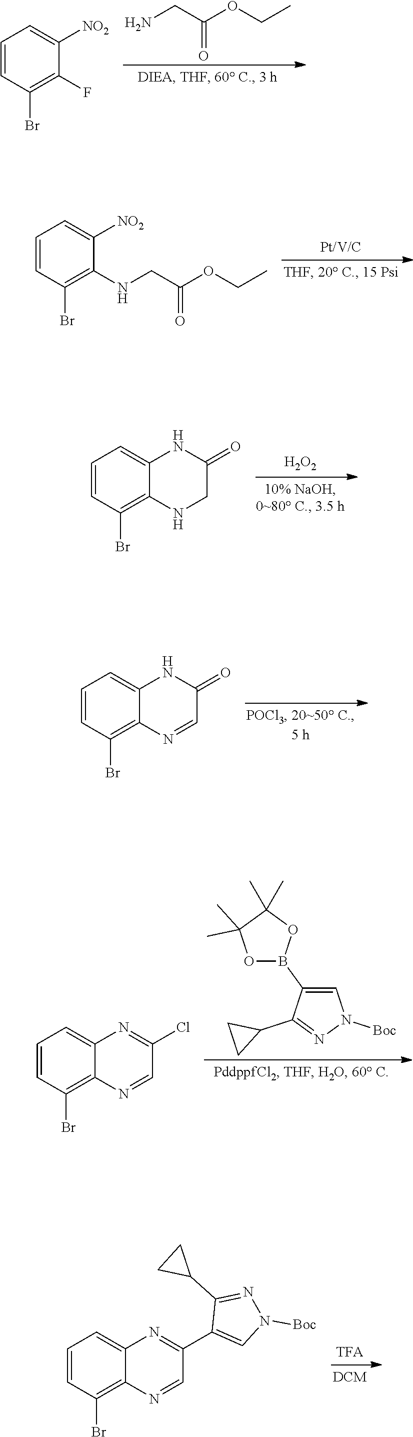

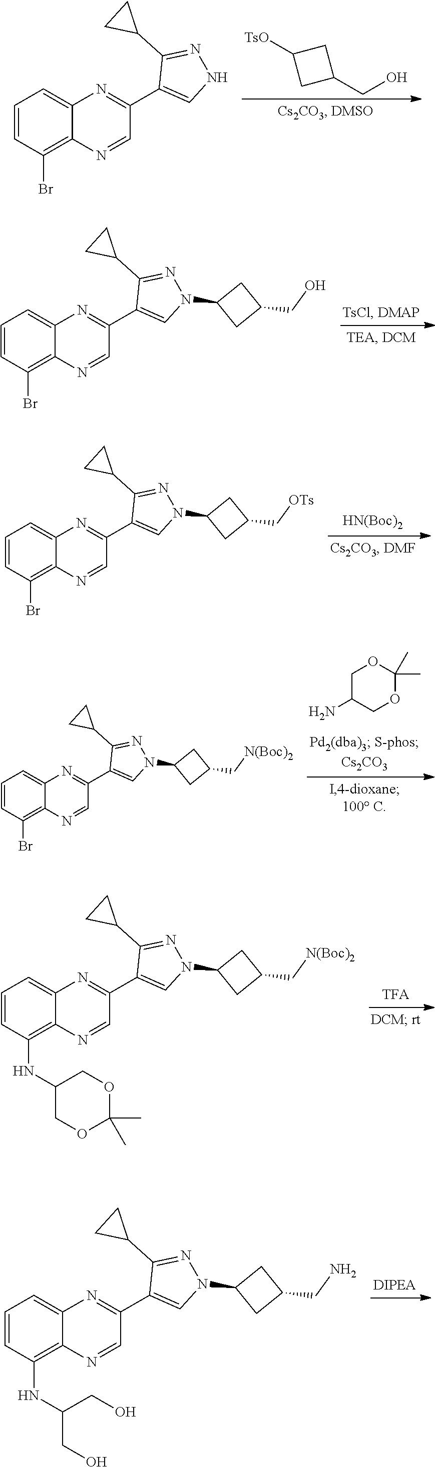

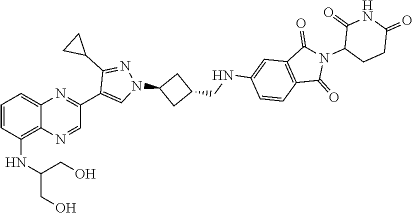 [SMILES: O=C1CCC(N2C(=O)c3ccc(NC[C@H]4C[C@H](n5cc(-c6cnc7c(NC(CO)CO)cccc7n6)c(C6CC6)n5)C4)cc3C2=O)C(=O)N1]

### Step 1. Synthesis of ethyl (2-bromo-6-nitrophenyl)glycinate

To a solution of 1-bromo-2-fluoro-3-nitro-benzene (84.0 g, 381 mmol) in THF (500 mL) were added ethyl 2-aminoacetate (60.0 g, 430 mmol) and DIEA (148 g, 1.15 mol, 200 mL) at 20 [symbol] . After the mixture was stirred at 60 [symbol] for 3 h, it was diluted with water (500 mL), and extracted with EtOAc (800 mL×2). The combined organic layers were washed with brine (500 mL), dried over Na₂SO₄, and concentrated. The residue was purified by column chromatography (petroleum ether:EtOAc=50:1 to 2:1) to provide the desired product (101 g, yield: 87%) as a yellow solid. ¹H NMR (400 MHz, DMSO-d₆) δ 7.98-7.81 (m, 2H), 6.91 (t, J=8.0 Hz, 1H), 6.68 (br t, J=6.0 Hz, 1H), 4.11-4.02 (m, 4H), 1.14 (t, J=7.2 Hz, 3H).

### Step 2. Synthesis of 5-bromo-3,4-dihydroquinoxalin-2 (1H)-one

To a solution of ethyl (2-bromo-6-nitrophenyl)glycinate (45.0 g, 148 mmol) in THF (450 mL) was added Pt/V/C (4.50 g, 17.2 mmol) at 20 [symbol] . After the mixture was stirred at 20 [symbol] for 2 h under H₂ at 15 psi, it was filtered with celite. The filtrate was concentrated to provide the desired product (35.0 g, yield: 86%) as a yellow solid. ¹H NMR (400 MHz, DMSO-d₆) δ 10.43 (s, 1H), 7.02 (dd, J=1.3, 8.0 Hz, 1H), 6.73 (dd, J=1.2, 7.8 Hz, 1H), 6.58-6.49 (m, 1H), 5.83-5.44 (m, 1H), 3.80 (s, 2H).

### Step 3. Synthesis of 5-bromoquinoxalin-2 (1H)-one

To a solution of 5-bromo-3,4-dihydroquinoxalin-2 (1H)-one (19.0 g, 83.7 mmol) in 10% NaOH solution (200 mL) was added H₂O₂ solution (30%, 80 mL, 833 mmol) at 0 [symbol] . The reaction mixture was stirred at 0 [symbol] for 1 h, 20 [symbol] for 2 h, and 80 [symbol] for 0.5 h. The mixture was cooled to 0 [symbol] . AcOH was added to adjust pH to ˜7. The solid was collected by filtration and dried in vacuo to provide the desired product (15.0 g, crude) as a yellow solid. MS (ESI) m/z=226.9 [M+H]⁺.

### Step 4. Synthesis of 5-bromo-2-chloroquinoxaline

A solution of 5-bromoquinoxalin-2 (1H)-one (15.0 g, 66.7 mmol) in POCl₃ (150 mL) was stirred at 50 [symbol] for 5 h. Then the reaction mixture was cooled to 20 [symbol] , poured into H₂O (300 mL) and extracted with EtOAc (200 mL×3). The combined organic layers were washed with brine (300 mL×2), dried over Na₂SO₄, and concentrated under reduce pressure. The residue was purified by column chromatography (petroleum ether:EtOAc=100:1 to 20:1) to provide the desired product (8.20 g, yield: 47%) as a yellow solid. ¹H NMR (400 MHz, DMSO-d₆) δ 9.09 (s, 1H), 8.25 (dd, J=1.2, 7.6 Hz, 1H), 8.06 (dd, J=1.2, 8.4 Hz, 1H), 7.86-7.79 (m, 1H).

The remaining steps were performed according to the procedures for steps 1 to 8 of GS-702 to provide the desired product (3.5 mg, yield: 3% over 8 steps) as a yellow solid. MS (ESI) m/z=665.5 [M+H]⁺.

### Example 70. 5-((Trans-3-((3-cyclopropyl-4-(5-fluoro-6-methylpyridin-2-yl)-1H-pyrazol-1-yl)methyl)cyclobutyl)amino)-2-(2,6-dioxopiperidin-3-yl)isoindoline-1,3-dione (GS-711)

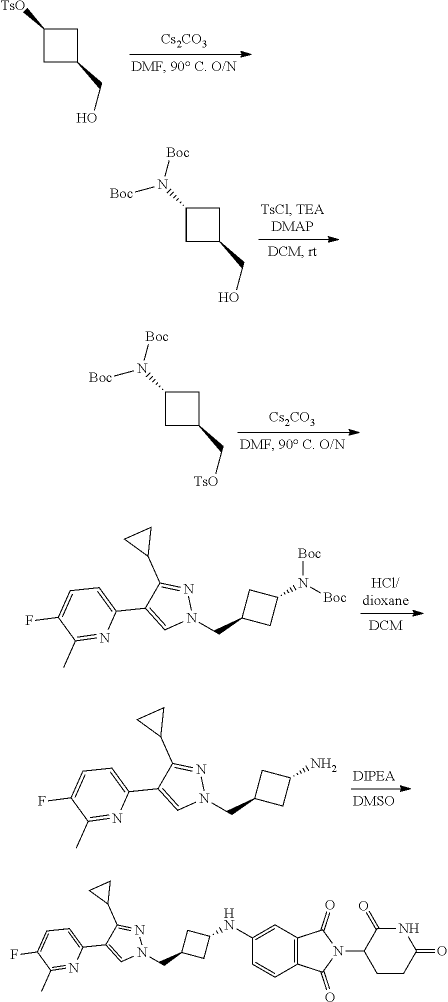

### Step 1. Synthesis of tert-butyl (tert-butoxycarbonyl)(trans-3-(hydroxymethyl)cyclobutyl)carbamate

To a mixture of cis-3-(hydroxymethyl)cyclobutyl 4-methylbenzenesulfonate (1.0 g, 3.9 mmol) and tert-butyl N-tert-butoxycarbonylcarbamate (1.27 g, 5.8 mmol) in DMF (10 mL) was added Cs₂CO₃ (2.54 g, 7.8 mmol). The reaction mixture was stirred at 90 [symbol] overnight, before it was quenched with water, and extracted with EtOAc. The organic layer was washed with water and brine, dried over Na₂SO₄, filtered and concentrated. The residue was purified by chromatography (petroleum ether:EtOAc=2:1) to afford the title compound (370 mg, yield: 31%) as a bright oil.

### Step 2. Synthesis of (trans-3-(bis(tert-butoxycarbonyl)amino)cyclobutyl)methyl 4-methylbenzenesulfonate

To a mixture of tert-butyl (tert-butoxycarbonyl)(trans-3-(hydroxymethyl)cyclobutyl)carbamate (370 mg, 1.2 mmol) and TsCl (365 mg, 1.8 mmol) in DCM (5 mL) was added TEA (248 mg, 2.4 mmol) and DMAP (15 mg, 12 μmol). The reaction mixture was stirred at rt overnight, before it was quenched with water, and extracted with DCM. The organic layer was washed with brine, dried over Na₂SO₄, filtered and concentrated. The residue was purified by reverse phase chromatography (0-70% MeCN in H₂O) to afford the title compound (crude, 500 mg, yield: 89%) as a yellow oil.

### Step 3. Synthesis of tert-butyl (tert-butoxycarbonyl)(trans-3-((3-cyclopropyl-4-(5-fluoro-6-methylpyridin-2-yl)-1H-pyrazol-1-yl)methyl)cyclobutyl)carbamate

To a mixture of 6-(3-cyclopropyl-1H-pyrazol-4-yl)-3-fluoro-2-methylpyridine (150 mg, 690 μmol) and (trans-3-(bis(tert-butoxycarbonyl)amino)cyclobutyl)methyl 4-methylbenzenesulfonate (377 mg, 828 μmol) in DMF (4 mL) was added Cs₂CO₃ (450 mg, 1.4 mmol). The reaction mixture was stirred at 90 [symbol] for 4 h, before it was quenched with water, and extracted with EtOAc. The organic layer was washed with water and brine, dried over Na₂SO₄, filtered and concentrated. The residue was purified by reverse phase chromatography (0-70% MeCN in H₂O) to afford the title compound (130 mg, yield: 38%) as a bright oil. MS (ESI) m/z=501.4 [M+H]⁺.

### Step 4. Synthesis of trans-3-((3-cyclopropyl-4-(5-fluoro-6-methylpyridin-2-yl)-1H-pyrazol-1-yl)methyl)cyclobutan-1-amine

To a stirred solution of tert-butyl (tert-butoxycarbonyl)(trans-3-((3-cyclopropyl-4-(5-fluoro-6-methylpyridin-2-yl)-1H-pyrazol-1-yl)methyl)cyclobutyl)carbamate (130 mg, 259 μmol) in DCM (0.5 mL) was added HCl/dioxane (0.5 mL). After the reaction mixture was stirred at rt for 10 min, it was concentrated to afford the title compound (50 mg, yield: 64%) as a white solid. MS (ESI) m/z=301.3 [M+H]⁺.

### Step 5. Synthesis of 5-((trans-3-((3-cyclopropyl-4-(5-fluoro-6-methylpyridin-2-yl)-1H-pyrazol-1-yl)methyl)cyclobutyl)amino)-2-(2.6-dioxopiperidin-3-yl)isoindoline-1,3-dione

To a mixture of trans-3-((3-cyclopropyl-4-(5-fluoro-6-methylpyridin-2-yl)-1H-pyrazol-1-yl)methyl)cyclobutan-1-amine (40 mg, 133 μmol) and 2-(2,6-dioxo-3-piperidyl)-5-fluoro-isoindoline-1,3-dione (73.57 mg, 266 μmol) in DMSO (1 mL) was added DIPEA (34 mg, 266 μmol). The reaction mixture was stirred at 120 [symbol] for 2 h, before it was purified by reverse phase chromatography (0-70% MeCN in H₂O) to afford the title compound (3.6 mg, yield: 5%) as a yellow solid. MS (ESI) m/z=557.4 [M+H]⁺.

### Example 71. 5-(((Trans-3-(3-cyclopropyl-4-(6-(3-hydroxyazetidin-1-yl)quinoxalin-2-yl)-1H-pyrazol-1-yl)cyclobutyl)methyl)amino)-2-(2,6-dioxopiperidin-3-yl)isoindoline-1,3-dione (GS-712)

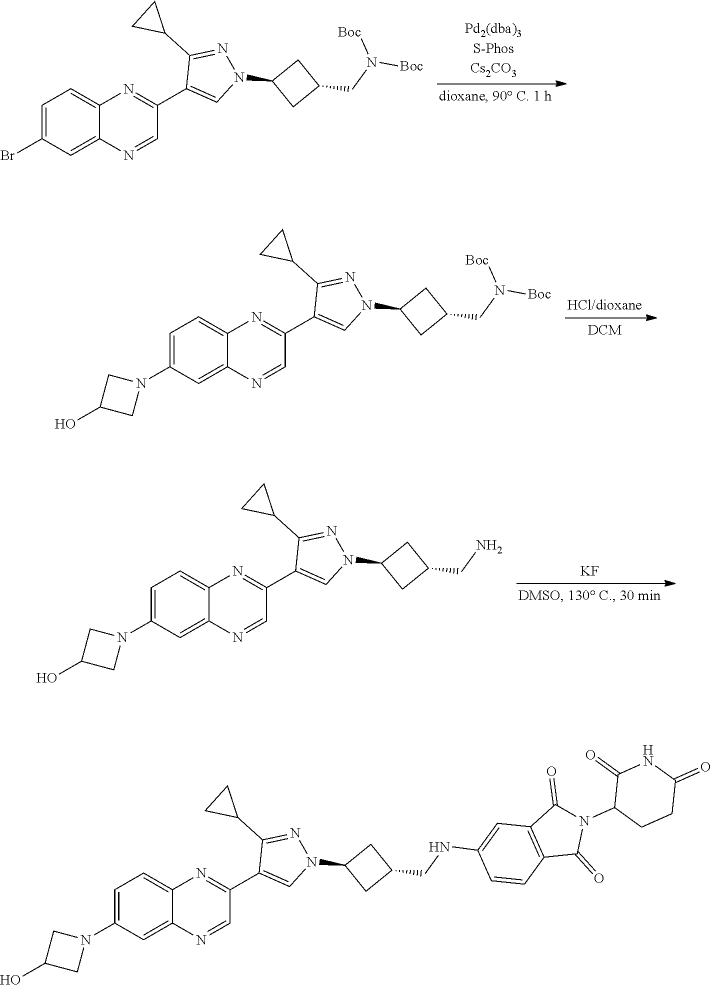 [SMILES: CC(C)(C)OC(=O)N(C[C@H]1C[C@H](n2cc(-c3cnc4cc(Br)ccc4n3)c(C3CC3)n2)C1)C(=O)OC(C)(C)C ; CC(C)(C)OC(=O)N(C[C@H]1C[C@H](n2cc(-c3cnc4cc(N5CC(O)C5)ccc4n3)c(C3CC3)n2)C1)C(=O)OC(C)(C)C ; NC[C@H]1C[C@H](n2cc(-c3cnc4cc(N5CC(O)C5)ccc4n3)c(C3CC3)n2)C1 ; "KF" ; O=C1CCC(N2C(=O)c3ccc(NC[C@H]4C[C@H](n5cc(-c6cnc7cc(N8CC(O)C8)ccc7n6)c(C6CC6)n5)C4)cc3C2=O)C(=O)N1]

### Step 1. Synthesis of tert-butyl (tert-butoxycarbonyl)((trans-3-(3-cyclopropyl-4-(6-(3-hydroxyazetidin-1-yl)quinoxalin-2-yl)-1H-pyrazol-1-yl)cyclobutyl)methyl)carbamate

To a mixture of tert-butyl N-[[trans-3-[4-(6-bromoquinoxalin-2-yl)-3-cyclopropyl-pyrazol-1-yl]cyclobutyl]methyl]-N-tert-butoxycarbonyl-carbamate (100 mg, 167 μmol) and azetidin-3-ol (24 mg, 334 μmol) in dioxane (4 mL) were added Pd₂(dba)₃ (15 mg, 17 μmol), S-Phos (14 mg, 33 μmol) and Cs₂CO₃ (109 mg, 334 μmol). The reaction mixture was stirred at 100° C. for 1 h, before it was quenched with water, and extracted with EtOAc. The organic phase was washed with water, dried over Na₂SO₄, filtered and concentrated. The residue was purified by silica gel chromatography (0-70% petroleum ether in EtOAc) to afford the title compound (80 mg, yield: 81%) as a yellow solid. MS (ESI) m/z=591.5 [M+H]⁺.

### Step 2. Synthesis of 1-(2-(1-(trans-3-(aminomethyl)cyclobutyl)-3-cyclopropyl-1H-pyrazol-4-yl)quinoxalin-6-yl)azetidin-3-ol

To a stirred solution of tert-butyl (tert-butoxycarbonyl)((trans-3-(3-cyclopropyl-4-(6-(3-hydroxyazetidin-1-yl)quinoxalin-2-yl)-1H-pyrazol-1-yl)cyclobutyl)methyl)carbamate (80 mg, 135 μmol) in DCM (0.5 mL) was added HCl/dioxane (0.5 mL). The reaction mixture was stirred at rt for 10 min, before it was concentrated to afford the title compound (30 mg, yield: 57%) as a yellow solid. MS (ESI) m/z=391.4 [M+H]⁺.

### Step 3. Synthesis of 5-(((trans-3-(3-cyclopropyl-4-(6-(3-hydroxyazetidin-1-yl)quinoxalin-2-yl)-1H-pyrazol-1-yl)cyclobutyl)methyl)amino)-2-(2,6-dioxopiperidin-3-yl)isoindoline-1,3-dione

To a mixture of 1-(2-(1-(trans-3-(aminomethyl)cyclobutyl)-3-cyclopropyl-1H-pyrazol-4-yl)quinoxalin-6-yl)azetidin-3-ol (30 mg, 76.8 μmol) and 2-(2,6-dioxo-3-piperidyl)-5-fluoro-isoindoline-1,3-dione (42 mg, 154 μmol) in DMSO (0.5 mL) was added KF (13 mg, 230 μmol). The reaction mixture was stirred at 120 [symbol] for 2 h, before it was purified by reverse phase chromatography (0-70% MeCN in H₂O) to afford the title compound (7.5 mg, yield: 15%) as a yellow solid. MS (ESI) m/z=487.4 [M+H]⁺.

### Example 72. 5-(((Trans-3-(4-cyclopropyl-3-(6-methylpyridin-2-yl)-1H-pyrazol-1-yl)cyclobutyl)methyl)amino)-2-(2,6-dioxopiperidin-3-yl)isoindoline-1,3-dione (GS-713)

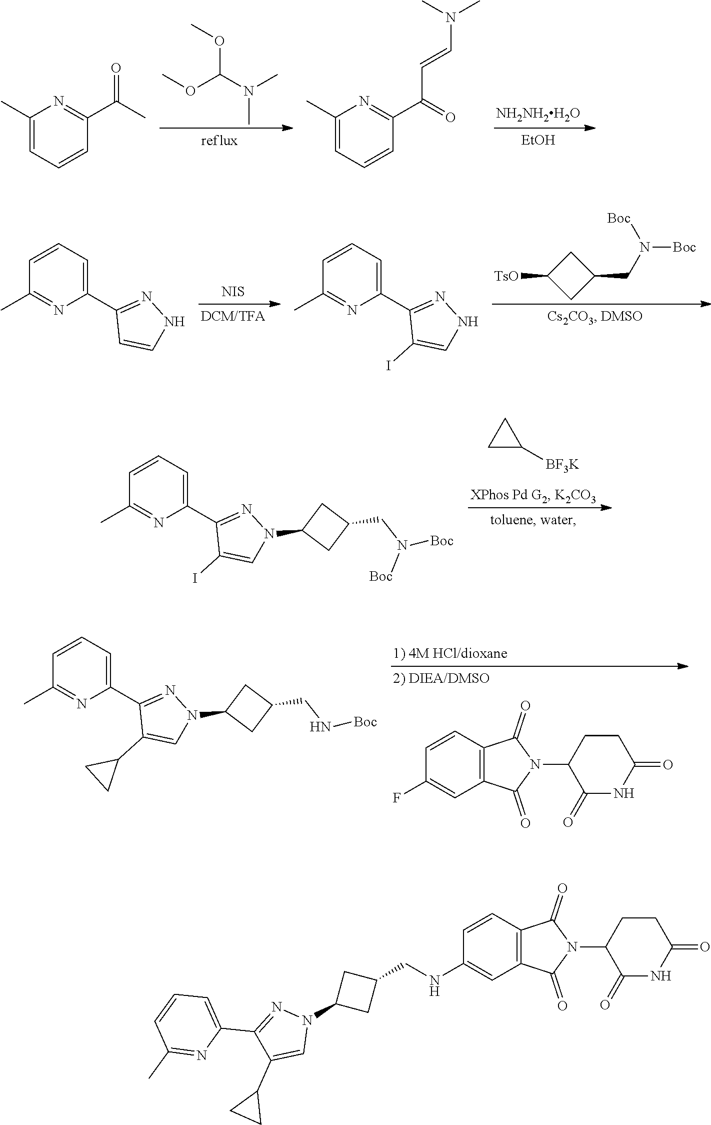

### Step 1. Synthesis of 3-(dimethylamino)-1-(6-methyl-2-pyridyl)prop-2-en-1-one

A mixture of 1-(6-methyl-2-pyridyl)ethanone (5 g, 36.99 mmol) and 1,1-dimethoxy-N,N-dimethyl-methanamine (7 g, 58.74 mmol) was heated at reflux overnight under N₂. The mixture was cooled and filtered to afford the title compound (5.0 g, yield: 71%) as a light yellow solid. MS (ESI) m/z=191.2 [M+H]⁺.

### Step 2. Synthesis of 2-methyl-6-(1H-pyrazol-3-yl)pyridine

To a solution of 3-(dimethylamino)-1-(6-methyl-2-pyridyl)prop-2-en-1-one (2.5 g, 13.14 mmol) in ethanol (30 mL) was added hydrated hydrazine (5 mL) at rt. After the reaction mixture was heated at reflux for 1 h, it was cooled to rt, and concentrated to afford the title compound (1.8 g, yield: 86%) as a white solid. MS (ESI) m/z=160.1 [M+H]⁺.

### Step 3. Synthesis of 2-(4-iodo-1H-pyrazol-3-yl)-6-methyl-pyridine

To a solution of 2-methyl-6-(1H-pyrazol-3-yl)pyridine (1.2 g, 7.54 mmol) in DCM (50 mL) and TFA (15 mL) was added 1-iodopyrrolidine-2,5-dione (2.54 g, 11.31 mmol) at rt. The mixture was stirred overnight, before it was concentrated. pH was adjusted to ˜10 with NaHCO₃. The resulting mixture was extracted with DCM (80 mL×2). The combined organic phase was washed with brine (80 mL), dried and concentrated. The residue was purified with reverse phase chromatography to afford the title compound (1.3 g, yield: 59%) as a light yellow solid. MS (ESI) m/z=286.0 [M+H]⁺.

### Step 4. Synthesis of tert-butyl (tert-butoxycarbonyl)((trans-3-(4-iodo-3-(6-methylpyridin-2-yl)-1H-pyrazol-1-yl)cyclobutyl)methyl)carbamate

A mixture of 2-(4-iodo-1H-pyrazol-3-yl)-6-methyl-pyridine (1.20 g, 4.08 mmol), cis-3-((bis(tert-butoxycarbonyl)amino)methyl)cyclobutyl 4-methylbenzenesulfonate(2.42 g, 5.31 mmol) and Cs₂CO₃ (2.66 g, 8.17 mmol) in DMSO (10 mL) was stirred at 90 [symbol] for 1 h. The mixture was cooled to rt and purified by reverse phase chromatography to afford the title compound (850 mg, yield: 33%) as a light brown solid. MS (ESI) m/z=569.3 [M+H]⁺.

### Step 5. Synthesis of tert-butyl ((trans-3-(4-cyclopropyl-3-(6-methylpyridin-2-yl)-1H-pyrazol-1-yl)cyclobutyl)methyl)carbamate

A mixture of tert-butyl (tert-butoxycarbonyl)((trans-3-(4-iodo-3-(6-methylpyridin-2-yl)-1H-pyrazol-1-yl)cyclobutyl)methyl)carbamate (400 mg, 626.27 μmol), cyclopropyl(trifluoro)boranuide (294.31 mg, 2.70 mmol), XPhos Pd G2 (100 mg, 626.27 μmol) and K₂CO₃ (250 mg, 1.81 mmol) in toluene (5 mL) and water (1 mL) was heated at 100 [symbol] under N₂ for 5 h under microwave irradiation. The mixture was cooled and concentrated. The residue was purified by reverse phase chromatography to afford the title compound (22 mg, yield: 7%) as a light brown solid. MS (ESI) m/z=383.5 [M+H]⁺.

### Step 6. Synthesis of 5-(((trans-3-(4-cyclopropyl-3-(6-methylpyridin-2-yl)-1H-pyrazol-1-yl)cyclobutyl)methyl)amino)-2-(2,6-dioxopiperidin-3-yl)isoindoline-1,3-dione

To a solution of tert-butyl ((trans-3-(4-cyclopropyl-3-(6-methylpyridin-2-yl)-1H-pyrazol-1-yl)cyclobutyl)methyl)carbamate (22 mg, 43.71 μmol) in DCM (2 mL) was added HCl (4 M in dioxane, 1.5 mL) at rt. The reaction mixture was stirred for 1 h, before it was concentrated. The resulting residue (25 mg, 90.51 μmol) and DIEA (45 mg, 348.84 μmol) in DMSO (2 mL) was heated at 130 [symbol] for 1 h under N₂ under microwave irradiation. The mixture was cooled to rt and purified by reverse phase chromatography to afford the title compound (3 mg, yield: 13%) as a yellow solid. MS (ESI) m/z=539.7 [M+H]⁺.

### Example 73. 5-(((Trans-3-(3-cyclopropyl-4-(1H-pyrazolo[3,4-b]pyridin-6-yl)-1H-pyrazol-1-yl)cyclobutyl)methyl)amino)-2-(2,6-dioxopiperidin-3-yl)isoindoline-1,3-dione (GS-714)

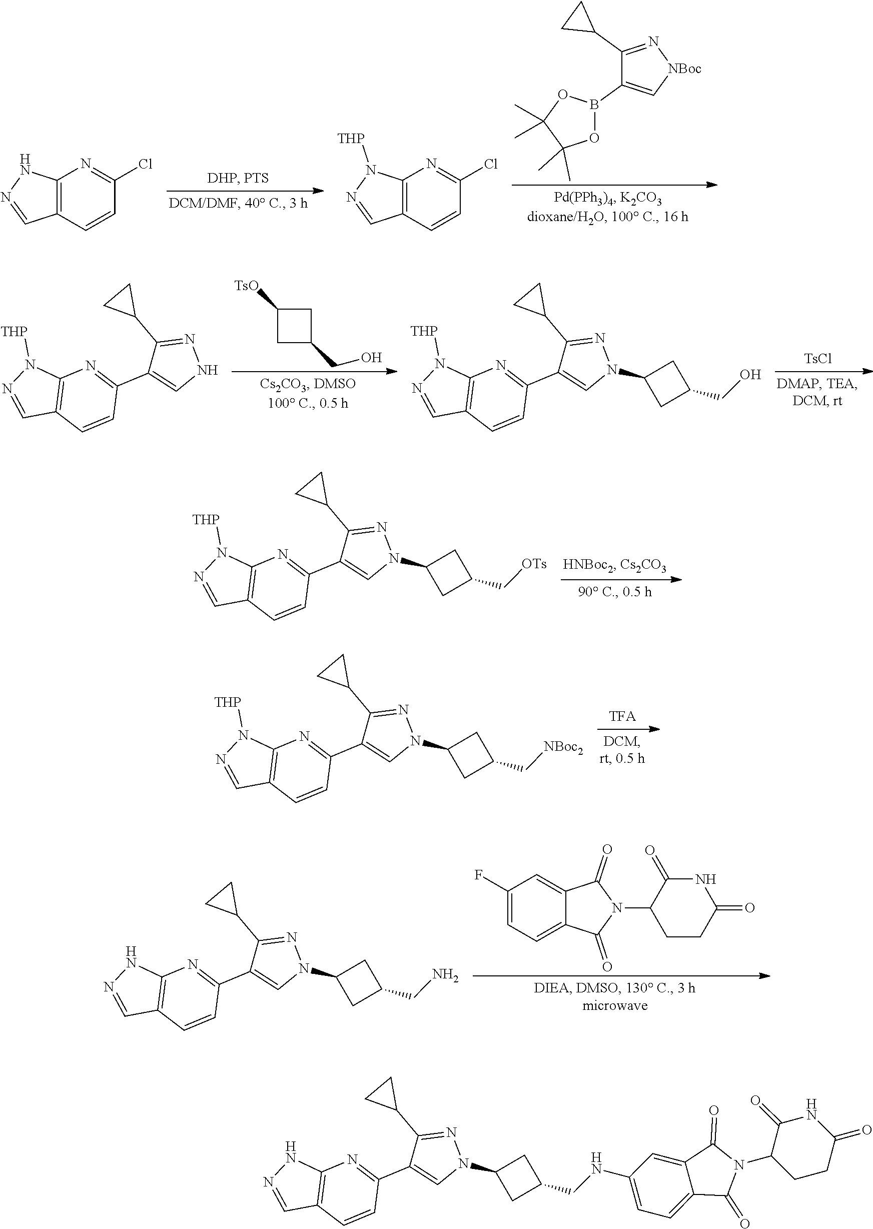

### Step 1. Synthesis of 6-chloro-1-(tetrahydro-2H-pyran-2-yl)-1H-pyrazolo[3,4-b]pyridine

To a solution of 6-chloro-1H-pyrazolo[3,4-b]pyridine (500 mg, 3.26 mmol) in DMF (2.0 mL) were added DHP (739.45 mg, 8.79 mmol, 798.54 μL) and PTS (112.13 mg, 651.17 μmol). The reaction mixture was stirred at 40 [symbol] for 3 h, before it was diluted with aqueous NaHCO₃ solution, and extracted with EtOAc (20 mL×3). The combined organic layers were washed with brine, dried over Na₂SO₄, filtered and concentrated. The residue was purified by silica gel chromatography (EtOAc:petroleum ether=7:3) to afford the title compound (720 mg, yield: 93%) as a light yellow oil.

### Step 2. Synthesis of 6-(3-cyclopropyl-1H-pyrazol-4-yl)-1-(tetrahydro-2H-pyran-2-yl)-1H-pyrazolo[3,4-b]pyridine

To a solution of 6-chloro-1-(tetrahydro-2H-pyran-2-yl)-1H-pyrazolo[3,4-b]pyridine (520 mg, 2.19 mmol) in 1,4-dioxane (7.5 mL) and water (3.0 mL) were added Pd(PPh₃)₄ (2.53 g, 2.19 mmol) and K₂CO₃ (302.36 mg, 2.19 mmol). The reaction mixture was stirred at 100 [symbol] for 16 h, before it was poured into ice/water and extracted with EtOAc (20 mL×2). The combined organic layers were washed with brine, dried over Na₂SO₄, filtered and concentrated. The residue was purified by reverse phase chromatography (MeOH:H₂O:TFA=50:50:0.5) to afford the title compound (327 mg, yield: 48%) as a white solid. MS (ESI) m/z=310.4 [M+H]⁺.

### Step 3. Synthesis of (trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methanol

To a solution of 6-(3-cyclopropyl-1H-pyrazol-4-yl)-1-(tetrahydro-2H-pyran-2-yl)-1H-pyrazolo[3,4-b]pyridine (327 mg, 1.05 mmol) and cis-3-(hydroxymethyl)cyclobutyl 4-methylbenzenesulfonate (323.01 mg, 1.26 mmol) in DM S 3.0 mL) was added Cs₂CO₃ (1.03 g, 3.15 mmol). The reaction mixture was stirred at 100 [symbol] for 0.5 h, before it was purified by reverse phase chromatography (MeOH:H₂O:TFA=60:40:0.5). The combined fractions were adjusted to pH=6˜7 with sat. NaHCO₃ solution. The mixture was extracted with EtOAc (20 mL×2). The combined organic layers were washed with brine, dried over Na₂SO₄, filtered and concentrated to afford the title compound (116.6 mg, yield: 28%) as a light oil. MS (ESI) m/z=394.6 [M+H]⁺.

### Step 4. Synthesis of (trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methyl 4-methylbenzenesulfonate

To a solution of (trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methanol (116.6 mg, 296.33 μmol) in DCM (1.5 mL) were added TsCl (112.99 mg, 592.66 μmol), DMAP (72.40 mg, 592.66 μmol) and TEA (149.93 mg, 1.48 mmol). The mixture was stirred at rt for 2 h, before it was poured into ice/water and extracted with EtOAc (20 mL×2). The combined organic layers were washed with brine, dried over Na₂SO₄, filtered and concentrated. The residue was purified by silica gel chromatography (EtOAc:petroleum ether=3:7) to afford the title compound (136 mg, yield: 84%) as a colorless oil. MS (ESI) m/z=548.6 [M+H]⁺.

### Step 5. Synthesis of tert-butyl (tert-butoxycarbonyl)((trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methyl)carbamate

To a solution of (trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methyl 4-methylbenzenesulfonate (136 mg, 248.33 μmol) and HNBoc₂ (59.35 mg, 273.16 μmol) in DMSO (1.5 mL) was added Cs₂CO₃ (161.82 mg, 496.65 μmol). The reaction mixture was stirred at 90 [symbol] for 0.5 h, before it was poured into ice/water, and extracted with EtOAc (20 mL×2). The combined organic layers were washed with brine, dried over Na₂SO₄, filtered and concentrated. The residue was purified by silica gel chromatography (EtOAc:petroleum ether=3:1) to afford the title compound (110 mg, yield: 75%) as a white solid. MS (ESI) m/z=593.8 [M+H]⁺.

### Step 6. Synthesis of (trans-3-(3-cyclopropyl-4-(1H-pyrazolo[3,4-b]pyridin-6-yl)-1H-pyrazol-1-yl)cyclobutyl)methanamine

To a solution of tert-butyl (tert-butoxycarbonyl)((trans-3-(3-cyclopropyl-4-(1-(tetrahydro-2H-pyran-2-yl)-1H-pyrazolo[3,4-b]pyridin-6-yl)-1H-pyrazol-1-yl)cyclobutyl)methyl)carbamate (110 mg, 185.58 μmol) in DCM (1.0 mL) was added TFA (21.16 mg, 185.58 μmol, 2.0 mL). The mixture was stirred at rt for 0.5 h, before it was concentrated to afford the title compound (TFA salt, 55 mg, yield: 70%) as a brown oil. MS (ESI) m/z=309.5 [M+H]⁺.

### Step 7. Synthesis of 5-(((trans-3-(3-cyclopropyl-4-(1H-pyrazolo[3,4-b]pyridin-6-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(1H-pyrazolo[3,4-b]pyridin-6-yl)-1H-pyrazol-1-yl)cyclobutyl)methanamine (55 mg, 178.35 μmol) and 2-(2,6-dioxo-3-piperidyl)-5-fluoro-isoindoline-1,3-dione (59.12 mg, 214.02 μmol) in DMSO (1.5 mL) was added DIPEA (461.01 mg, 3.57 mmol, 0.621 mL). The mixture was stirred at 130 [symbol] for 3 h under microwave irradiation, before it was purified by reverse phase chromatography (MeOH:H₂O:TFA=70:30:0.5) and prep-TLC (DCM:MeOH=20:1) to afford the title compound (22.88 mg, yield: 23%) as a yellow solid. MS (ESI) m/z=565.39 [M+H]⁺.

### Example 74. 5-((Trans-3-(3-cyclopropyl-4-(5-fluoro-6-methylpyridin-2-yl)-1H-pyrazol-1-yl)cyclobutyl)methoxy)-2-(2,6-dioxopiperidin-3-yl)isoindoline-1,3-dione (GS-715)

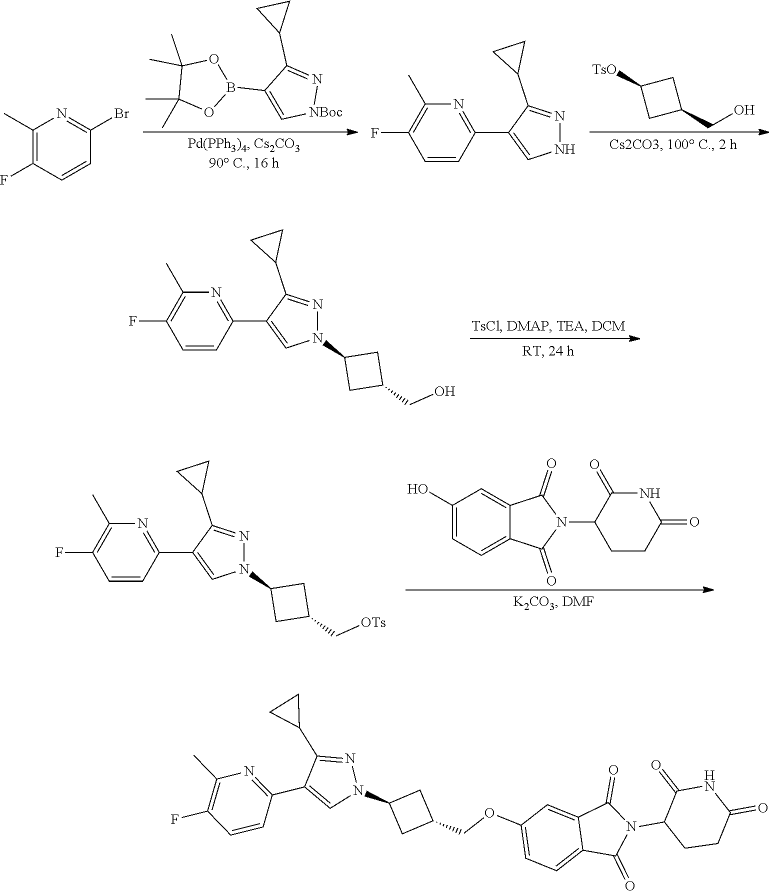

### Step 1. Synthesis of 6-(3-cyclopropyl-1H-pyrazol-4-yl)-3-fluoro-2-methylpyridine

To a stirred solution of 6-bromo-3-fluoro-2-methylpyridine (500.0 mg, 2.63 mmol) and tert-butyl 3-cyclopropyl-4-(4,4,5,5-tetramethyl-1,3,2-dioxaborolan-2-yl)pyrazole-1-carboxylate(1.36 g, 3.96 mmol) in 1,4-dioxane (25 mL) and H₂O (2.5 mL) were added Pd(dppf)Cl₂ (192.3n 0.26 mmol) and K₂CO₃ (1.08 g, 7.89 mmol) under Ar. The mixture was stirred at 100 [symbol] overnight. Water (100 mL) was added. The mixture was extracted with EtOAc (100 mL×3). The combined organic layers were washed with brine, dried over Na₂SO₄, filtered and concentrated. The residue was purified by silica gel chromatography to afford the title compound (540 mg, yield: 95%) as a solid. MS (ESI) m/z=218.4 [M+H]⁺.

### Step 2 to 3. Synthesis of (trans-3-(3-cyclopropyl-4-(5-fluoro-6-methylpyridin-2-yl)-1H-pyrazol-1-yl)cyclobutyl)methyl 4-methylbenzenesulfonate

The title compound was synthesized following the procedures for steps 3 to 4 of GS-714 (405 mg, yield: 36% over 2 steps) as a solid. MS (ESI) m/z=456.4 [M+H]⁺.

### Step 4. Synthesis of 5-((trans-3-(3-cyclopropyl-4-(5-fluoro-6-methylpyridin-2-yl)-1H-pyrazol-1-yl)cyclobutyl)methoxy)-2-(2,6-dioxopiperidin-3-yl)isoindoline-1,3-dione

To a stirred solution of (trans-3-(3-cyclopropyl-4-(5-fluoro-6-methylpyridin-2-yl)-1H-pyrazol-1-yl)cyclobutyl)methyl 4-methylbenzenesulfonate (90.3 mg, 0.329 mmol) in DMF (5 mL) was added K₂CO₃ (90.6 mg, 0.657 mmol). The mixture was stirred at 65 [symbol] for 1.5 h. Water (100 mL) was added. The mixture was extracted with EtOAc (30 mL×3). The combined organic layers were washed with brine, dried over Na₂SO₄, filtered and concentrated. The residue was purified by silica gel chromatography to afford the title compound (25 mg, yield: 21%) as a solid. MS (ESI) m/z=558.8 [M+H]⁺.

### Example 75. 5-((Trans-3-(3-cyclopropyl-4-(5-fluoro-6-methylpyridin-2-yl)-1H-pyrazol-1-yl)cyclobutyl)methoxy)-2-(2,6-dioxopiperidin-3-yl)isoindoline-1,3-dione (GS-716)

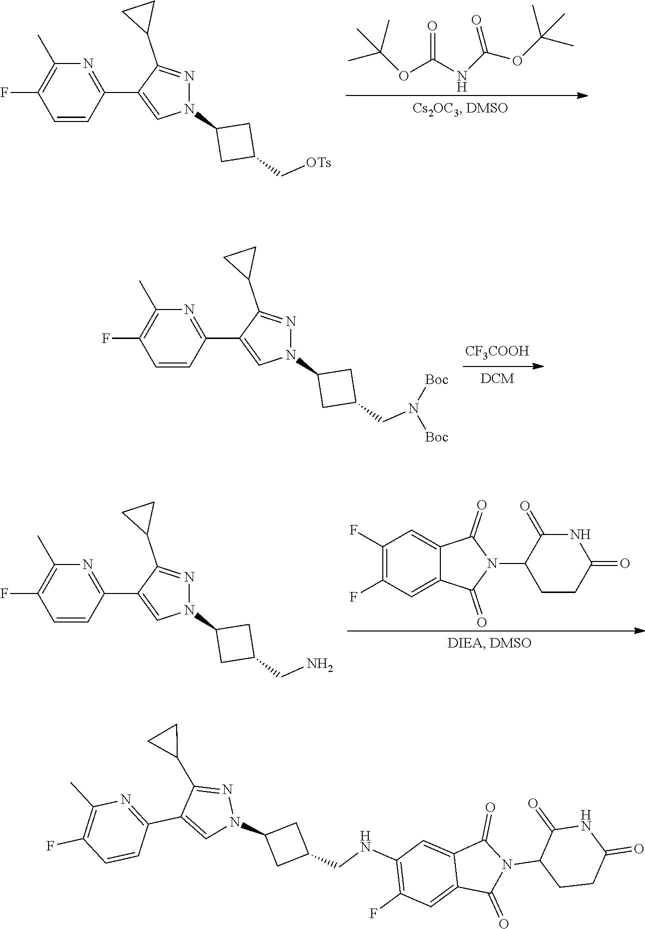

GS-716 was synthesized following the procedures for steps 5 to 7 of GS-695 (45 mg, yield: 9% over 3 steps) as a yellow solid. MS (ESI) m/z=575.7 [M+H]⁺.

### Example 76. 5-(((Trans-3-(3-cyclopropyl-4-(5-fluoro-3-methylpyridin-2-yl)-1H-pyrazol-1-yl)cyclobutyl)methyl)amino)-2-(2,6-dioxopiperidin-3-yl)isoindoline-1,3-dione (GS-717)

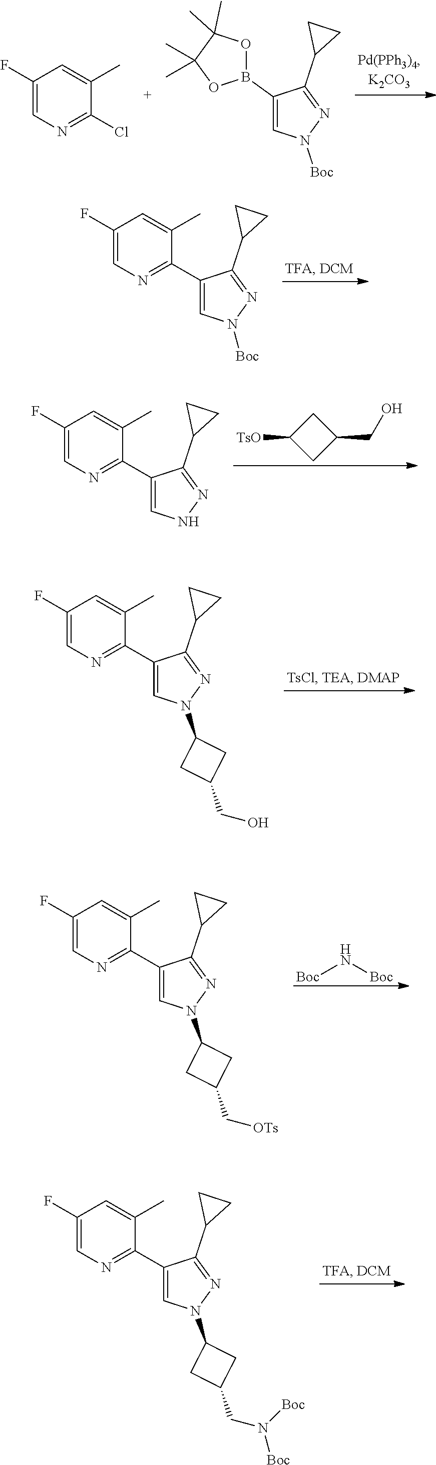

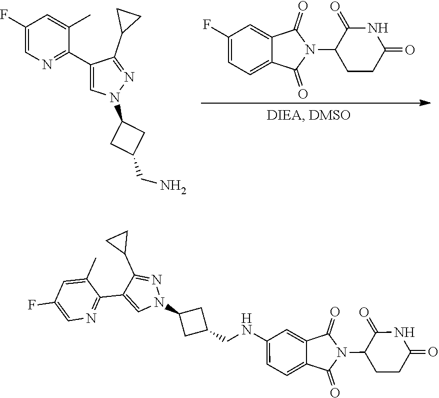 [SMILES: Cc1cc(F)cnc1-c1cn([C@H]2C[C@H](CN)C2)nc1C1CC1 ; O=C1CCC(N2C(=O)c3ccc(F)cc3C2=O)C(=O)N1 ; Cc1cc(F)cnc1-c1cn([C@H]2C[C@H](CNc3ccc4c(c3)C(=O)N(C3CCC(=O)NC3=O)C4=O)C2)nc1C1CC1]

### Step 1. Synthesis of tert-butyl 3-cyclopropyl-4-(5-fluoro-3-methylpyridin-2-yl)-1H-pyrazole-1-carboxylate

To a solution of 2-chloro-5-fluoro-3-methylpyridine (500.0 mg, 3.44 mmol) and tert-butyl 3-cyclopropyl-4-(4,4,5,5-tetramethyl-1,3,2-dioxaborolan-2-yl)-1H-pyrazole-1-carboxylate (1148.9 mg, 3.44 mmol) in 1,4-dixoane (20 mL) and H₂O (5 mL) were added Pd(PPh₃)₄ (392.7 mg, 0.34 mmol) and K₂CO₃ (1434.4 mg, 10.32 mmol). After the mixture was stirred at 90 [symbol] under N₂ for 3 h, it was concentrated under reduced pressure. The residue was purified by silica gel chromatography (petroleum ether:EtOAc=4:1) to afford the title compound (304.9 mg, yield: 28%) as a white solid. MS (ESI) m/z=318.6 [M+H]⁺.

### Step 2. Synthesis of 2-(3-cyclopropyl-1H-pyrazol-4-yl)-5-fluoro-3-methylpyridine

To a stirred solution of tert-butyl 3-cyclopropyl-4-(5-fluoro-3-methylpyridin-2-yl)-1H-pyrazole-1-carboxylate (304.9 mg, 0.96 mmol) in DCM (6 mL) was added TFA (3 mL). The mixture was stirred at rt for 1 h, before it was concentrated. The residue was purified by silica gel chromatography to afford the title compound (203.2 mg, yield: 98%) as a light colorless oil. MS (ESI) m/z=218.5 [M+H]⁺.

### Step 3. Synthesis of (trans-3-(3-cyclopropyl-4-(S-fluoro-3-methylpyridin-2-yl)-1H-pyrazol-1-yl)cyclobutyl)methanol

To a solution of 2-(3-cyclopropyl-1H-pyrazol-4-yl)-5-fluoro-3-methylpyridine (203.2 mg, 0.93 mmol) and cis-3-(hydroxymethyl)cyclobutyl 4-methylbenzenesulfonate (238.3 mg, 0.93 mmol) in DMSO (10 mL) was added Cs₂CO₃ (906.7 mg, 2.79 mmol). The mixture was stirred at 95 [symbol] for 1 h, before it was diluted with EtOAc (10 mL) and washed with water and brine. The organic layer was concentrated, and the residue was purified by silica gel chromatography to afford the title compound (152.6 mg, yield: 54%) as a white solid. MS (ESI) m/z=302.6 [M+H]⁺.

### Step 4. Synthesis of (trans-3-(3-cyclopropyl-4-(5-fluoro-3-methylpyridin-2-yl)-1H-pyrazol-1-yl)cyclobutyl)methyl 4-methylbenzenesulfonate

To a solution of (trans-3-(3-cyclopropyl-4-(5-fluoro-3-methylpyridin-2-yl)-1H-pyrazol-1-yl)cyclobutyl)methanol (152.6 mg, 0.51 mmol) and TsCl (290.7 mg, 1.53 mmol) in DCM (10 mL) were added DMAP (62.2 mg, 0.51 mmol) and TEA (518.1 mg, 5.13 mmol). After the mixture was stirred at rt for 3 h, it was diluted with DCM (10 mL), and washed with water and brine. The organic layer was concentrated and the residue was purified silica gel chromatography to afford the title compound (124.6 mg, yield: 53%) as a white solid. MS (ESI) m/z=456.7 [M+H]⁺.

### Step 5. Synthesis of tert-butyl (tert-butoxycarbonyl)((trans-3-(3-cyclopropyl-4-(5-fluoro-3-methylpyridin-2-yl)-1H-pyrazol-1-yl)cyclobutyl)methyl)carbamate

To a solution of (trans-3-(3-cyclopropyl-4-(5-fluoro-3-methylpyridin-2-yl)-1H-pyrazol-1-yl)cyclobutyl)methyl 4-methylbenzenesulfonate (124.6 mg, 0.27 mmol) and HNBoc₂ (53.3 mg, 0.27 mmol) in DMSO (5 mL) was added Cs₂CO₃ (263.2 mg, 0.81 mmol). The mixture was stirred at 100 [symbol] for 30 min, before it was diluted with EtOAc (10 mL) and washed with water and brine. The organic layer was concentrated and the residue was purified by silica gel chromatography to afford the title compound (111.7 mg, yield: 82%) as a white solid. MS (ESI) m/z=501.9 [M+H]⁺.

### Step 6. Synthesis of (trans-3-(3-cyclopropyl-4-(5-fluoro-3-methylpyridin-2-yl)-1H-pyrazol-1-yl)cyclobutyl)methanamine

To a stirred solution of tert-butyl (tert-butoxycarbonyl)((trans-3-(3-cyclopropyl-4-(5-fluoro-3-methylpyridin-2-yl)-1H-pyrazol-1-yl)cyclobutyl)methyl)carbamate (111.7 mg, 0.22 mmol) in DCM (4 mL) was added TFA (2 mL). After the mixture was stirred at rt for 1 h, it was concentrated. The residue was purified by silica gel chromatography to afford the title compound (60.6 mg, yield: 910%) as a light yellow oil. MS (ESI) m/z=301.6 [M+H]⁺.

### Step 7. Synthesis of 5-(((trans-3-(3-cyclopropyl-4-(5-fluoro-3-methylpyridin-2-y)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(5-fluoro-3-methylpyridin-2-yl)-1H-pyrazol-1-yl)cyclobutyl)methanamine (60.6 mg, 0.20 mmol) in DMSO (5 mL) was added 2-(2,6-dioxopiperidin-3-yl)-5-fluoroisoindoline-1,3-dione (55.2 mg, 0.22 mmol) and DIEA (85.1 mg, 0.66 mmol). After the mixture was stirred at 130 [symbol] under microwave irradiation for 2 h, it was purified by reverse phase chromatography (ACN in 0.05% NH₄HCO₃ solution) to afford the title compound (25.4 mg, yield: 20%) as a yellow solid. MS (ESI) m/z=557.8 [M+H]⁺.

### Example 77. 5-((3-(Trans-3-(3-cyclopropyl-4-(quinoxalin-2-yl)-1H-pyrazol-1-yl)cyclobutyl)propyl)amino)-2-(2,6-dioxopiperidin-3-yl)-6-fluoroisoindoline-1,3-dione (GS-718)

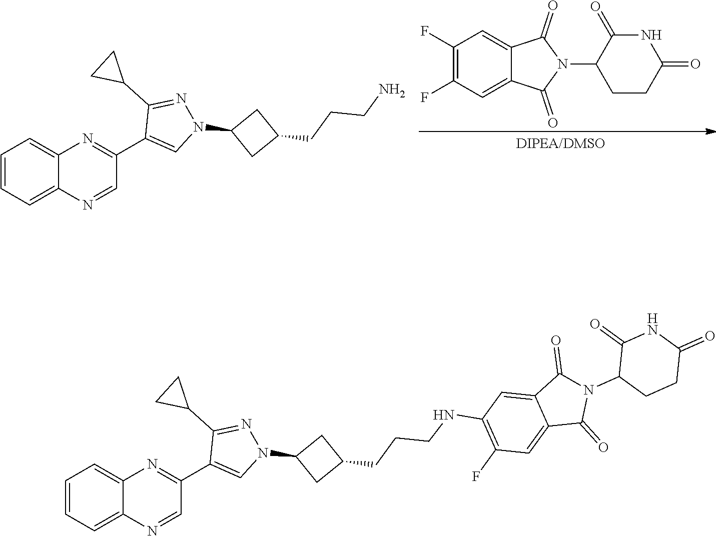

GS-718 was synthesized following the procedures for step 7 of GS-695 (280 mg, yield: 69%). MS (ESI) m/z=622.7 [M+H]⁺.

### Example 78. 5-(((Trans-3-(3-(Difluoromethyl)-4-(5-fluoro-3-methylpyridin-2-yl)-1H-pyrazol-1-yl)cyclobutyl)methyl)amino)-2-(2,6-dioxopiperidin-3-yl)isoindoline-1,3-dione (GS-719)

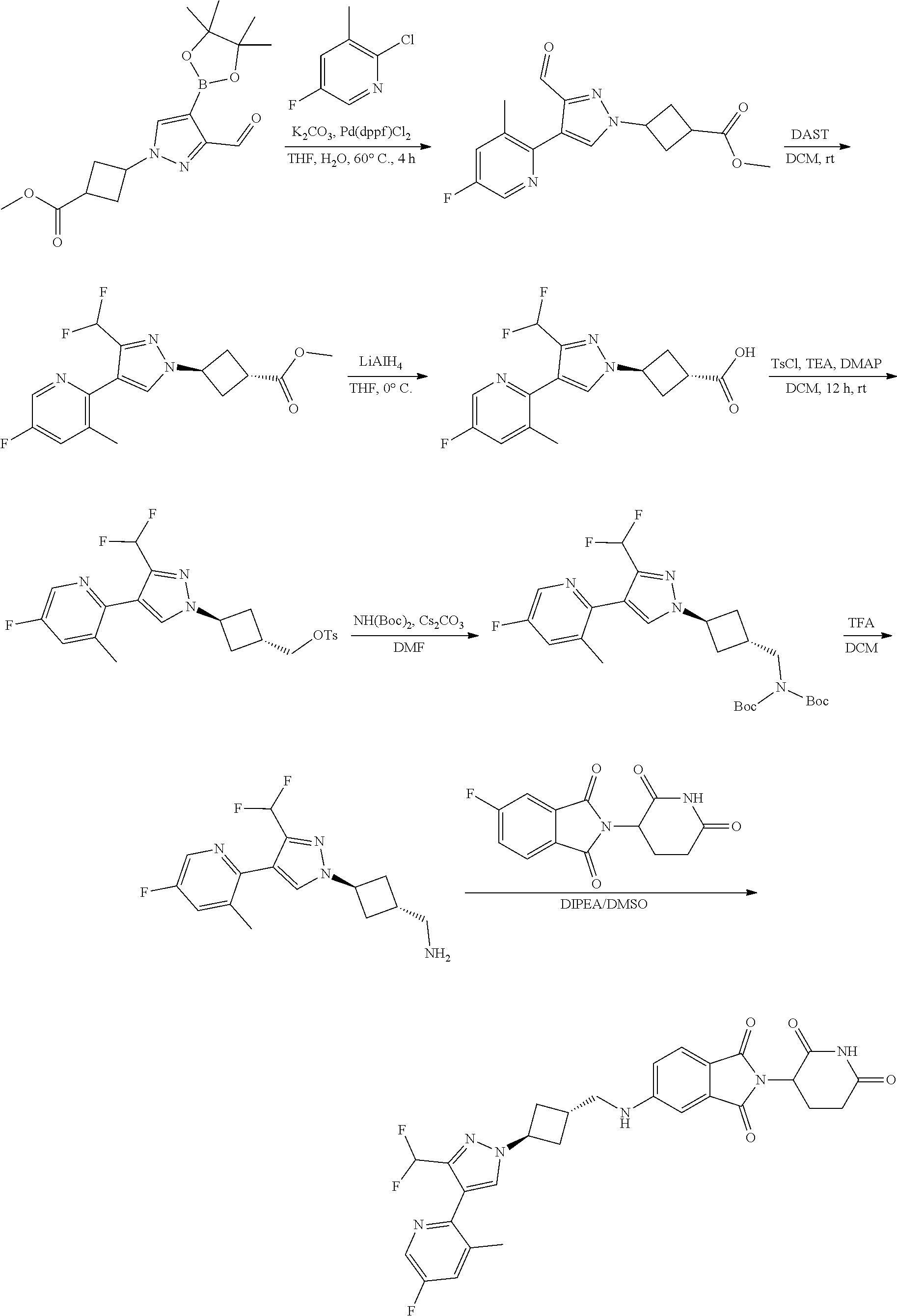

GS-719 was synthesized following the procedures for steps 1 to 3 of GS-677 and steps 4 to 7 of GS-695 (280 mg, yield: 3% over 7 steps) as a yellow solid. MS (ESI) m/z=567.54 [M+H]⁺.

### Example 79. 5-(((Trans-3-(3-cyclopropyl-4-(1H-pyrrolo[3,2-b]pyridin-5-yl)-1H-pyrazol-1-yl)cyclobutyl)methyl)amino)-2-(2,6-dioxopiperidin-3-yl)isoindoline-1,3-dione (GS-720)

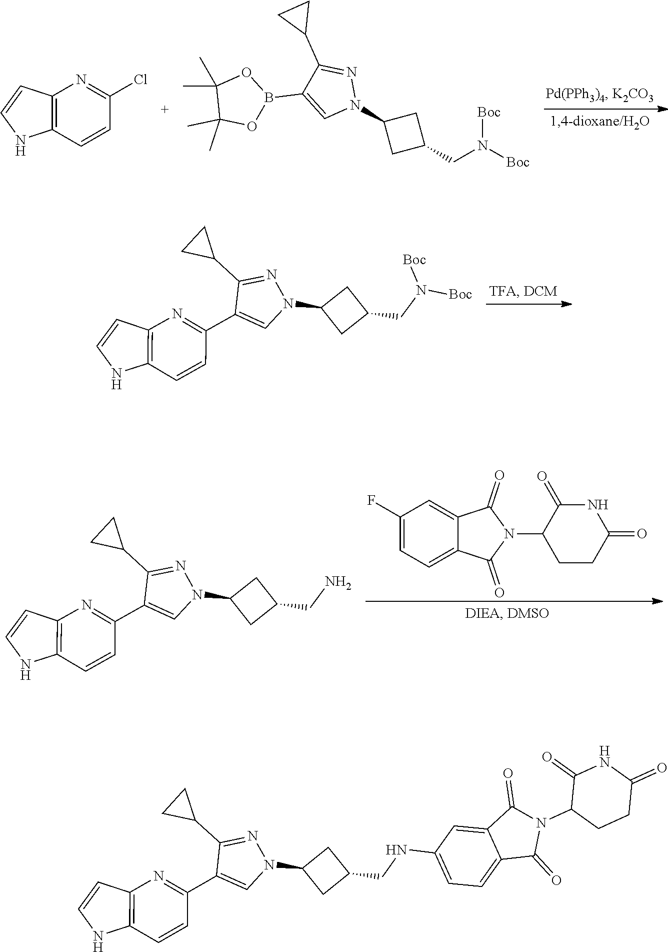

GS-720 was synthesized following the procedures for steps 2 to 4 of GS-661 (5.1 mg, yield: 1% over 3 steps) as a yellow solid. MS (ESI) m/z=564.8 [M+H]⁺.

### Example 80. 5-(((Trans-3-(3-cyclopropyl-4-(1H-pyrrolo[2,3-b]pyridin-6-yl)-1H-pyrazol-1-yl)cyclobutyl)methyl)amino)-2-(2,6-dioxopiperidin-3-yl)isoindoline-1,3-dione (GS-721)

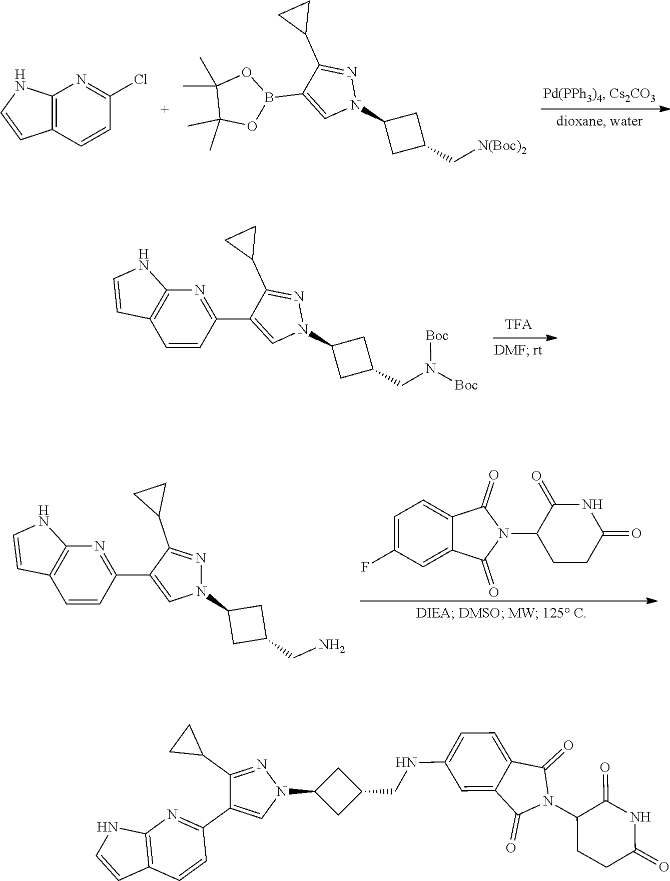

GS-721 was synthesized following the procedures for steps 2 to 4 of GS-661 (1.4 mg, yield: 1% over 3 steps) as a yellow solid. MS (ESI) m/z=564.4 [M+H]⁺.

### Example 81. 5-(((Trans-3-(3-cyclopropyl-4-(1H-pyrrolo[2,3-b]pyridin-6-yl)-1H-pyrazol-1-yl)cyclobutyl)methyl)amino)-2-(2,6-dioxopiperidin-3-yl)isoindoline-1,3-dione (GS-722)

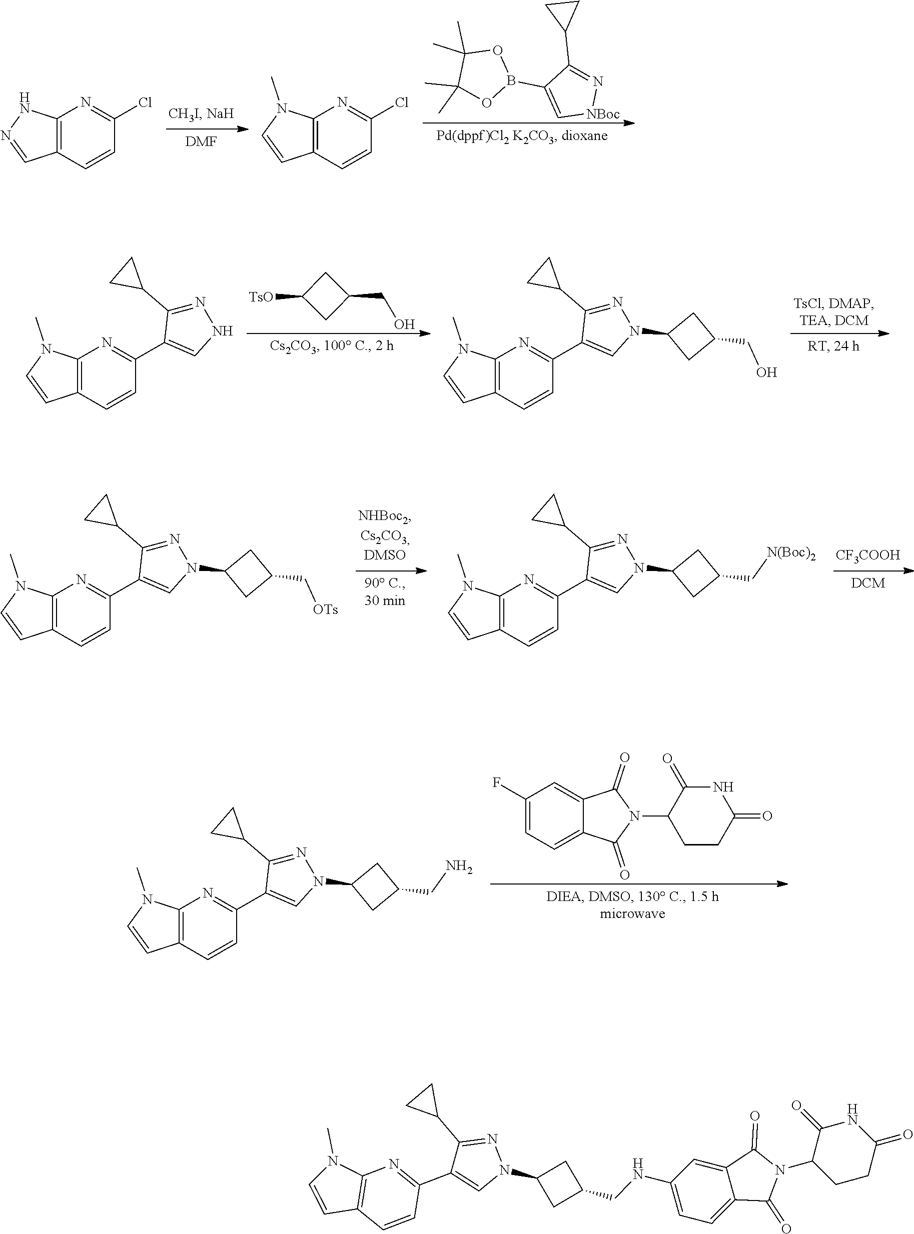

### Step 1. Synthesis of 6-chloro-1-methyl-1H-pyrrolo[2,3-b]pyridine

To a solution of 6-chloro-1H-pyrrolo[2,3-b]pyridine (1.0 g, 6.58 mmol) in DMF (10 mL) was added NaH (60% in mineral oil, 263.5 mg, 9.87 mmol) at 0 [symbol] , The mixture was stirred for 10 min, before iodomethane (1.4 g, 9.87 mmol) was added. The mixture was stirred at rt for 1 h, before it was quenched with water (10 mL). The mixture was dried over Na₂SO₄, filtered and concentrated to afford the title compound (450 mg, yield: 41%) as a colorless solid. MS (ESI) m/z=167.2 [M+H]⁺.

### Step 2 to 7. Synthesis of 5-(((trans-3-(3-cyclopropyl-4-(1-methyl-1H-pyrrolo[2,3-b]pyridin-6-yl)-1H-pyrazol-1-yl)cyclobutyl)methyl)amino)-2-(2,6-dioxopiperidin-3-yl)isoindoline-1,3-dione

GS-722 was synthesized following the procedures for steps 2 to 7 of GS-714 (14.0 mg, yield: 13% over 6 steps) as a yellow solid. MS (ESI) m/z=578.7 [M+H]⁺.

### Example 82. 5-(((Trans-3-(3-cyclopropyl-4-(5-fluoro-6-methylpyridin-2-yl)-1H-pyrazol-1-yl)-1-fluorocyclobutyl)methyl)amino)-2-(2,6-dioxopiperidin-3-yl)isoindoline-1,3-dione (GS-723)

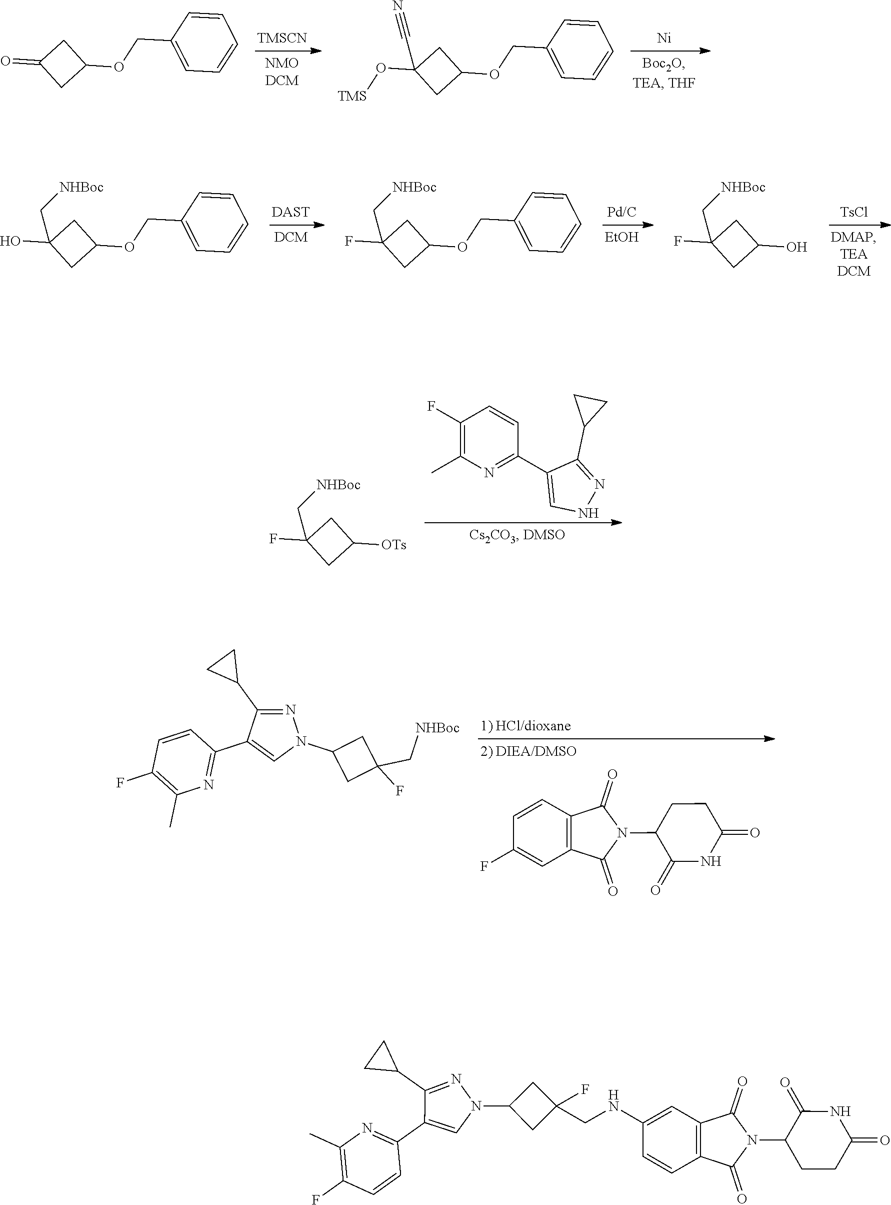

### Step 1. Synthesis of 3-benzyloxy-1-trimethylsilyloxy-cyclobutanecarbonitrile

To a solution of 3-benzyloxycyclobutanone (2 g, 11.35 mmol) in DCM (20 mL) were added trimethylsilylformonitrile (1.60 g, 16.13 mmol) and NMO (100 mg, 851.06 μmol) at rt. The mixture was stirred overnight. The mixture was diluted with DCM (100 mL), washed with sat. NaHCO₃ solution (50 mL) and brine (50 mL), dried and concentrated to afford the title compound (3.0 g, yield: 96%) as a light yellow oil. ¹H NMR (400 MHz, CDCl₃) δ 7.40-7.28 (m, 5H), 4.45 (s, 2H), 3.99-3.96 (m, 1H), 3.05-3.01 (m, 2H), 2.39-2.35 (m, 2H), 0.25 (s, 9H).

### Step 2. Synthesis of tert-butyl ((3-(benzyloxy)-1-hydroxycyclobutyl)methyl)carbamate

A mixture of 3-benzyloxy-1-trimethylsilyloxy-cyclobutanecarbonitrile (250 mg, 907.71 μmol), tert-butoxycarbonyl tert-butyl carbonate (300 mg, 1.37 mmol), TEA (200 mg, 1.98 mmol) and Ni (200 mg, 3.41 mmol) in THF (10 mL) was stirred under H₂ at rt overnight. The mixture was filtered and concentrated. The residue was purified by reverse phase chromatography to afford the title compound (250 mg, yield: 90%) as a colorless oil. ¹H NMR (400 MHz, CDCl₃) δ 7.36-7.28 (m, 5H), 4.99 (br s, 1H), 4.42 (s, 2H), 3.73-3.70 (m, 1H), 3.16 (s, 2H), 2.53-2.48 (m, 2H), 2.07-2.02 (m, 2H), 1.45 (s, 9H).

### Step 3. Synthesis of tert-butyl ((3-(benzyloxy)-1-fluorocyclobutyl)methyl)carbamate

To a solution of tert-butyl ((3-(benzyloxy)-1-hydroxycyclobutyl)methyl)carbamate (600 mg, 1.95 mmol) in DCM (10 mL) was added DAST (629 mg, 3.90 mmol) at 0 [symbol] . The mixture was stirred at ˜10 [symbol] for 2 h. The mixture was poured into sat. Na₂CO₃ solution (50 mL) and extracted with DCM (40 mL×2). The combined organic layers were dried and concentrated. The residue was purified by silica gel chromatography to afford the title compound (338 mg, yield: 42%) as a color less oil. MS (ESI) m/z=210.2 [M+H-100]⁺.

### Step 4. Synthesis of tert-butyl ((1-fluoro-3-hydroxycyclobutyl)methyl)carbamate

A mixture of tert-butyl ((3-(benzyloxy)-1-fluorocyclobutyl)methyl)carbamate (338 mg, 819.39 μmol) and Pd/C (200 mg) in ethanol (10 mL) was heated at 60 [symbol] under H₂ overnight. The reaction was monitored by TLC. Upon completion, the mixture was cooled to rt, filtered and concentrated to afford the title compound (230 mg, yield: 96%) as a colorless oil.

### Step 5. Synthesis of 3-(((tert-butoxycarbonyl)amino)methyl)-3-fluorocyclobutyl 4-methylbenzenesulfonate

To a solution of tert-butyl ((1-fluoro-3-hydroxycyclobutyl)methyl)carbamate (230 mg, 786.76 μmol) in DCM (10 mL) were added TEA (250 mg, 2.47 mmol), DMAP (50 mg, 409.28 μmol) and 4-methylbenzenesulfonyl chloride (260 mg, 1.36 mmol) at rt. The reaction mixture was stirred overnight, before it was diluted with DCM (50 mL). The mixture was washed with water (30 mL), sat. Na₂CO₃ solution (30 mL) and brine (30 mL). The organic phase was dried, filtered and concentrated to afford the title compound (340 mg, yield: 99%) as a light brown oil. MS (ESI) m/z=274.1 [M+H-100]⁺.

### Step 6. Synthesis of tert-butyl ((trans-3-(3-cyclopropyl-4-(S-fluoro-6-methylpyridin-2-yl)-1H-pyrazol-1-yl)-1-fluorocyclobutyl)methyl)carbamate

A mixture of 6-(3-cyclopropyl-1H-pyrazol-4-yl)-3-fluoro-2-methylpyridine (50 mg, 230.16 μmol), 3-(((tert-butoxycarbonyl)amino)methyl)-3-fluorocyclobutyl 4-methylbenzenesulfonate (200 mg, 444.52 μmol) and Cs₂CO₃ (150 mg, 460.41 μmol) in DMSO (2 mL) was heated at 90 [symbol] overnight. The mixture was cooled to rt and purified by reverse phase chromatography to afford the title compound (30 mg, yield: 30%) as a colorless oil. MS (ESI) m/z=419.4 [M+H]⁺.

### Step 7. Synthesis of 5-(((trans-3-(3-cyclopropyl-4-(5-fluoro-6-methylpyridin-2-yl)-1H-pyrazol-1-yl)-1-fluorocyclobutyl)methyl)amino)-2-(2,6-dioxopiperidin-3-yl)isoindoline-1,3-dione

To a solution of tert-butyl ((trans-3-(3-cyclopropyl-4-(5-fluoro-6-methylpyridin-2-yl)-1H-pyrazol-1-yl)-1-fluorocyclobutyl)methyl)carbamate (30 mg, 69.54 μmol) in DCM (5 mL) was added HCl (4 M in dioxane, 2.5 mL) at rt. The mixture was stirred for 1 h, before it was concentrated. The resulting residue, 2-(2,6-dioxo-3-piperidyl)-5-fluoro-isoindoline-1,3-dione (40 mg, 144.81 μmol) and DIEA (55 mg, 426.36 μmol) in DMSO (2 mL) was heated at 130 [symbol] for 5 h. The mixture was cooled to rt and purified by reverse phase chromatography and prep-TLC to afford the title compound (6 mg, yield: 15%) as a yellow solid. MS (ESI) m/z=575.8 [M+H]⁺.

### Example 83. 5-((2-(6-(3-Cyclopropyl-4-(quinoxalin-2-yl)-1H-pyrazol-1-yl)spiro[3.3]heptan-2-yl)ethyl)amino)-2-(2,6-dioxopiperidin-3-yl)isoindoline-1,3-dione (GS-724)

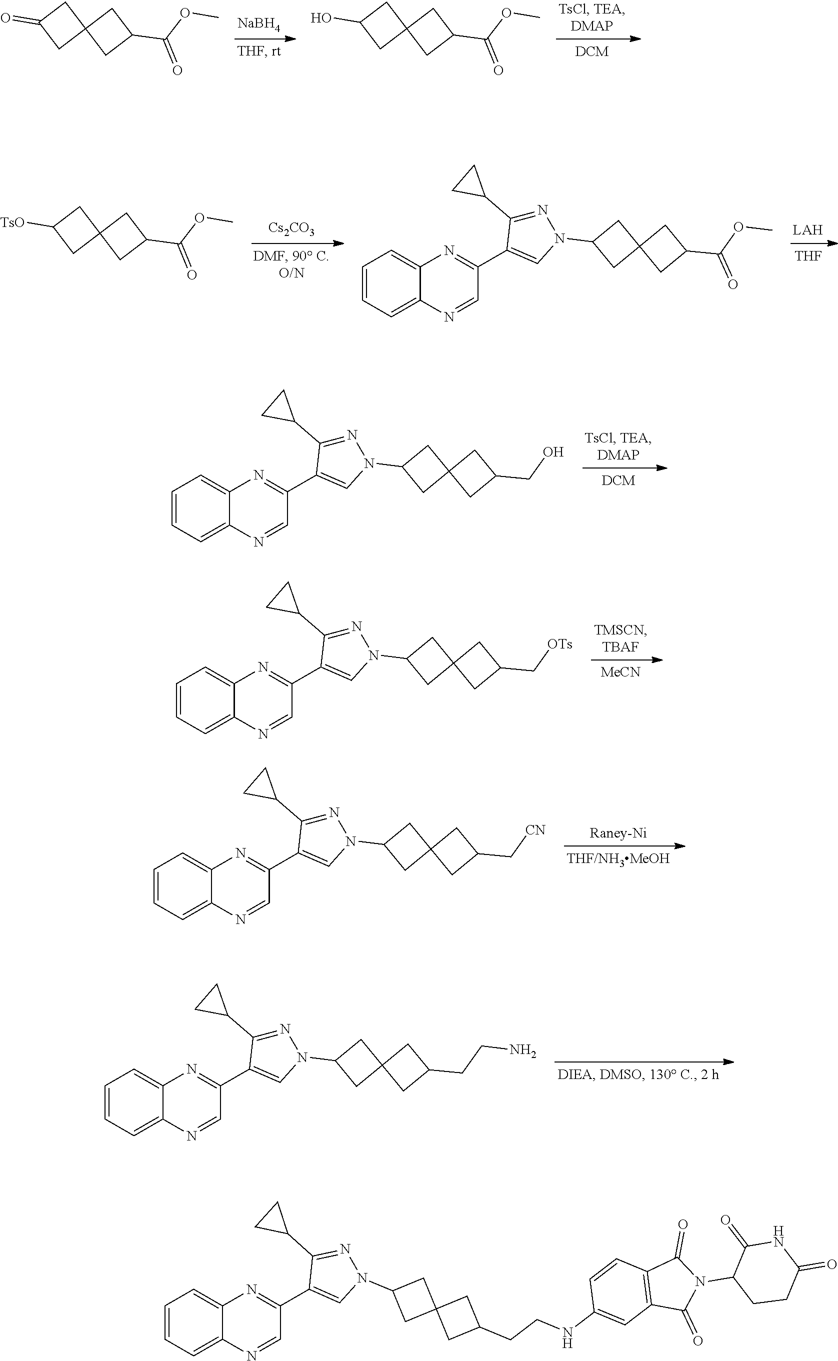

### Step 1. Synthesis of methyl 6-hydroxyspiro[3.3]heptane-2-carboxylate

To a solution of methyl 2-oxospiro[3.3]heptane-6-carboxylate (10 g, 59.5 mmol) in THF (100 mL) and MeOH (5 mL) was added NaBH₄ (1.8 g, 47.6 mmol) at 0 [symbol] . The reaction mixture was stirred at rt for 20 min, before it was quenched with water, and extracted with EtOAc. The organic layer was washed with water and brine, dried over Na₂SO₄, filtered and concentrated to afford the title compound (10 g, yield: 99%) as a bright oil. MS (ESI) m/z=171.2 [M+H]⁺.

### Step 2. Synthesis of methyl 6-(tosyloxy)spiro[3.3]heptane-2-carboxylate

To a solution of methyl 6-hydroxyspiro[3.3]heptane-2-carboxylate (9.5 g, 55.8 mmol) and TEA (11.3 g, 111.6 mmol) in DCM (150 mL) were added TsCl (13.8 g, 72.6 mmol) and DMAP (682 mg, 5.6 mmol). The reaction mixture was stirred at rt for 3 h, before it was quenched with water, and extracted with EtOAc. The organic layer was washed with aqueous Na₂CO₃ and brine. The organic phase was dried over Na₂SO₄, filtered and concentrated to afford the title compound (17.8 g, yield: 98%) as a bright oil.

### Step 3. Synthesis of methyl 6-(3-cyclopropyl-4-(quinoxalin-2-yl)-1H-pyrazol-1-yl)spiro[3.3]heptane-2-carboxylate

To a mixture of 2-(3-cyclopropyl-1H-pyrazol-4-yl)quinoxaline (5.0 g, 21.2 mmol) and methyl 6-(tosyloxy)spiro[3.3]heptane-2-carboxylate (8.9 g, 27.5 mmol) in DMF (100 mL) was added Cs₂CO₃ (13.8 g, 42.3 mmol). After the reaction mixture was stirred at 90 [symbol] overnight, it was quenched with water, and extracted with EtOAc. The organic phase was washed with water, dried over Na₂SO₄, filtered and concentrated. The residue was purified by reverse phase chromatography (0-70% MeCN in H₂O) to afford the title compound (4 g, yield: 49%) as a brown solid. MS (ESI) m/z=389.3 [M+H]⁺.

### Step 4. Synthesis of (6-(3-cyclopropyl-4-(quinoxalin-2-yl)-1H-pyrazol-1-yl)spiro[3.3]heptan-2-yl)methanol

To a stirred solution of methyl 6-(3-cyclopropyl-4-(quinoxalin-2-yl)-1H-pyrazol-1-yl)spiro[3.3]heptane-2-carboxylate (5.1 g, 13.1 μmol) in THF (80 mL) was added lithium triethylborohydride (2.77 g, 26.2 mmol) at 0 [symbol] . The reaction mixture was stirred at rt for 30 min, before it was quenched with water. The resulting suspension was filtered and the filtrate was concentrated. The residue was purified by silica gel chromatography (DCM:MeOH=0:1 to 1:9) to afford the title compound (4.2 g, yield: 89%) as a white solid. MS (ESI) m/z=361.3 [M+H]⁺.

### Step 5. Synthesis of (6-(3-cyclopropyl-4-(quinoxalin-2-yl)-1H-pyrazol-1-yl)spiro[3.3]heptan-2-yl)methyl 4-methylbenzenesulfonate

To a solution of (6-(3-cyclopropyl-4-(quinoxalin-2-yl)-1H-pyrazol-1-yl)spiro[3.3]heptan-2-yl)methanol (4.2 g, 11.6 μmol) in DCM (5 mL) were added TsCl (2.9 g, 15.1 mmol) and DMAP (142 mg, 1.2 mmol). The reaction mixture was stirred at rt for 2 h, before it was quenched with water, and extracted with EtOAc. The organic layer was washed with aqueous Na₂CO₃ and brine. The organic phase was dried over Na₂SO₄, filtered and concentrated to afford the title compound (4.6 g, yield: 77%) as a white solid. MS (ESI) m/z=515.2 [M+H]⁺.

### Step 6. Synthesis of 2-(6-(3-cyclopropyl-4-(quinoxalin-2-yl)-1H-pyrazol-1-yl)spiro[3.3]heptan-2-yl)acetonitrile

To a solution of (6-(3-cyclopropyl-4-(quinoxalin-2-yl)-1H-pyrazol-1-yl)spiro[3.3]heptan-2-yl)methyl 4-methylbenzenesulfonate (4.6 g, 8.9 mmol) in MeCN (40 mL) were added TMSCN (100 mg, 17.9 mmol) and TBAF (7.0 g, 26.8 mmol). The reaction mixture was stirred at 50 [symbol] overnight under N₂, before it was quenched with water, and extracted with EtOAc. The organic layer was washed with water and brine, dried over Na₂SO₄, filtered and concentrated. The residue was purified by reverse phase chromatography (0-70% MeCN in H₂O) to afford the title compound (2.2 g, yield: 67%) as a white solid. MS (ESI) m/z=370.3 [M+H]⁺.

### Step 7. Synthesis of 2-(6-(3-cyclopropyl-4-(quinoxalin-2-yl)-1H-pyrazol-1-yl)spiro[3.3]heptan-2-yl)ethan-1-amine

To a solution of 2-(6-(3-cyclopropyl-4-(quinoxalin-2-yl)-1H-pyrazol-1-yl)spiro[3.3]heptan-2-yl)acetonitrile (2.1 g, 5.1 mmol) in NH₃/methanol (2 mL) and THE (10 mL) was added Raney-Ni (2.0 g). The reaction mixture was purged with H₂ three times and stirred under H₂ at rt for 2 h. The mixture was filtered and concentrated to afford the title compound (2.1 g, yield: 99%) as a yellow solid. MS (ESI) m/z=374.3 [M+H]⁺.

### Step 8. Synthesis of 5-((2-(6-(3-cyclopropyl-4-(quinoxalin-2-yl)-1H-pyrazol-1-yl)spiro[3.3]heptan-2-yl)ethyl)amino)-2-(2,6-dioxopiperidin-3-yl)isoindoline-1,3-dione

To a mixture of 2-(6-(3-cyclopropyl-4-(quinoxalin-2-yl)-1H-pyrazol-1-yl)spiro[3.3]heptan-2-yl)ethan-1-amine (2.1 g, 5.6 mmol) and 2-(2,6-dioxo-3-piperidyl)-5-fluoro-isoindoline-1,3-dione (2.3 g, 8.4 mmol) in DMSO (30 mL) was added DIPEA (1.5 g, 11.3 mmol). After the reaction mixture was stirred at 130 [symbol] for 2 h, it was purified by reverse phase chromatography (0-70% MeCN in H₂O) to afford the title compound (733 mg, yield: 210%) as a yellow solid. MS (ESI) m/z=630.5 [M+H]⁺.

### Example 84. 3-(6-(2-(Trans-3-(3-cyclopropyl-4-(quinoxalin-2-yl)-1H-pyrazol-1-yl)cyclobutyl)ethyl)-1-oxoisoindolin-2-yl)piperidine-2,6-dione (GS-725)

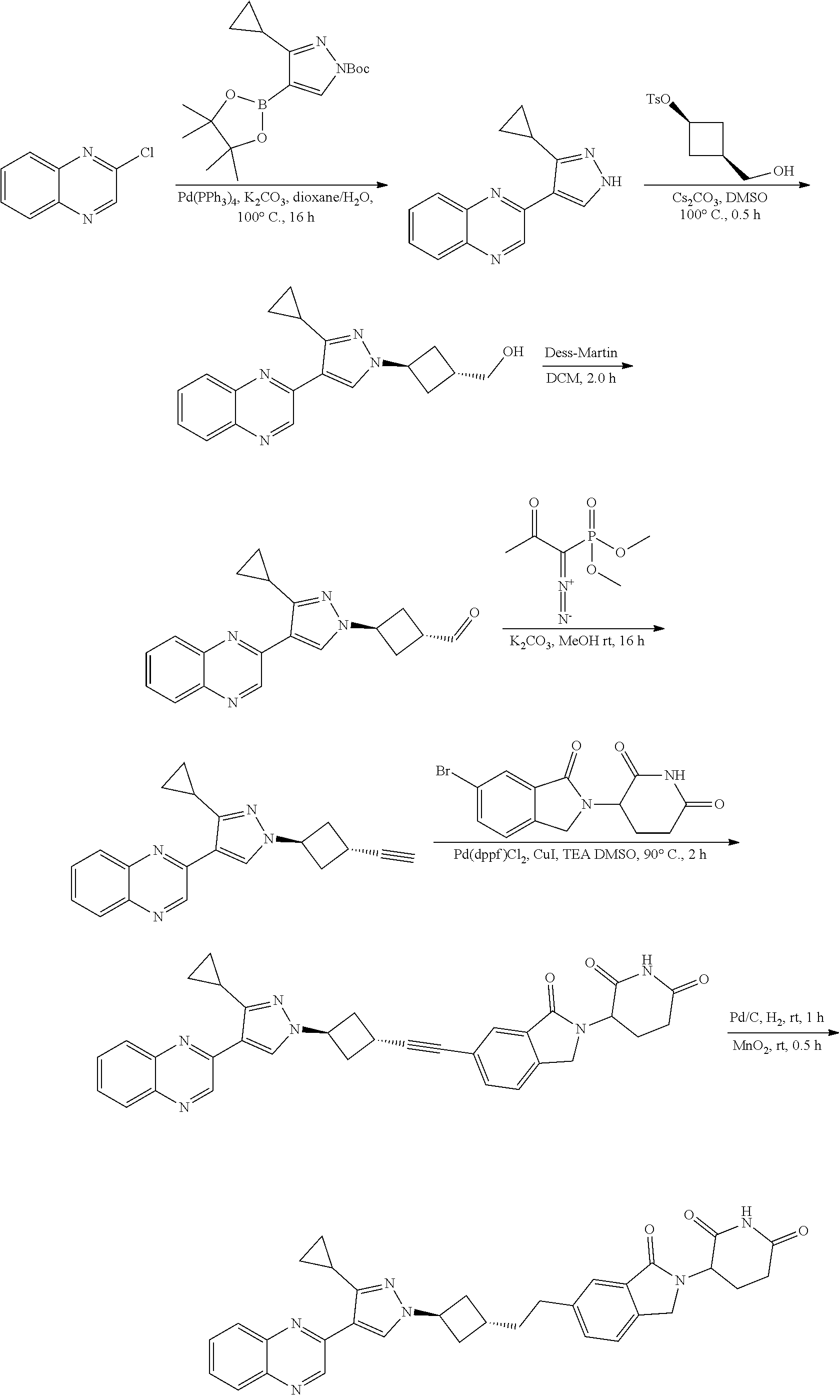

### Step 1. Synthesis of 2-(3-cyclopropyl-1H-pyrazol-4-yl)quinoxaline

To a solution of 2-chloroquinoxaline (1.0 g, 6.08 mmol) and tert-butyl 3-cyclopropyl-4-(4,4,5,5-tetramethyl-1,3,2-dioxaborolan-2-yl)pyrazole-1-carboxylate (2.44 g, 7.29 mmol) in 1,4-dioxane (15 mL) and water (15 mL) were added Pd(PPh₃)₄ (702.08 mg, 607.56 μmol) and K₂CO₃ (2.52 g, 18.23 mmol). The reaction was stirred at 100 [symbol] for 16 h under Ar, before it was purified by reverse phase chromatography (MeOH:H₂O:TFA=60:40:0.5) to afford the title compound (720 mg, yield: 50%) as a light solid. MS (ESI) m/z=237.6 [M+H]⁺.

### Step 2. Synthesis of (trans-3-(3-cyclopropyl-4-(quinoxalin-2-yl)-1H-pyrazol-1-yl)cyclobutyl)methanol

To a solution of 2-(3-cyclopropyl-1H-pyrazol-4-yl)quinoxaline (400 mg, 1.69 mmol) and cis-3-(hydroxymethyl)cyclobutyl 4-methylbenzenesulfonate (520.72 mg, 2.03 mmol) in DMSO (4.0 mL) was added Cs₂CO₃ (1.65 g, 5.08 mmol). The reaction mixture was stirred at 100 [symbol] for 0.5 h, before it was purified by reverse phase chromatography (MeOH:H₂O:TFA=80:20:0.5) to afford the title compound (210 mg, yield: 39%) as a light oil. MS (ESI) m/z=321.6 [M+H]⁺.

### Step 3. Synthesis of trans-3-(3-cyclopropyl-4-(quinoxalin-2-yl)-1H-pyrazol-1-yl)cyclobutane-1-carbaldehyde

To a solution of (trans-3-(3-cyclopropyl-4-(quinoxalin-2-yl)-1H-pyrazol-1-yl)cyclobutyl)methanol (210 mg, 655.46 μmol) in DCM (2.0 mL) was added Dess-Martin periodinane (556.01 mg, 1.31 mmol). The reaction mixture was stirred at rt for 2 h, before it was filtered. The filtrate was concentrated to afford the title product, which was used directly in the next step without further purification. MS (ESI) m/z=319.5 [M+H]⁺.

### Step 4. Synthesis of 2-(3-cyclopropyl-1-(trans-3-ethynylcyclobutyl)-1H-pyrazol-4-yl)quinoxaline

To a solution of trans-3-(3-cyclopropyl-4-(quinoxalin-2-yl)-1H-pyrazol-1-yl)cyclobutane-1-carbaldehyde (200 mg, 628.20 μmol) and 1-diazo-1-dimethoxyphosphoryl-propan-2-one (144.82 mg, 753.84 μmol) in MeOH (5.0 mL) was added K₂CO₃ (260.46 mg, 1.88 mmol). The reaction mixture was stirred at rt for 16 h, before it was concentrated. The residue was extracted with EtOAc (20 mL×2). The combined organic layers were washed with brine, dried with Na₂SO₄ and filtered. The filtrate was concentrated and purified by silica gel chromatography (EtOAc:petroleum ether=30%) to afford the title compound (38 mg, yield: 19%) as a white solid. MS (ESI) m/z=315.6 [M+H]⁺.

### Step 5. Synthesis of 3-(6-((trans-3-(3-cyclopropyl-4-(quinoxalin-2-yl)-1H-pyrazol-1-yl)cyclobutyl)ethynyl)-1-oxoisoindolin-2-yl)piperidine-2,6-dione

To a solution of 2-(3-cyclopropyl-1-(trans-3-ethynylcyclobutyl)-1H-pyrazol-4-yl)quinoxaline (37.80 mg, 120.23 μmol) and 3-(6-bromo-1-oxo-isoindolin-2-yl)piperidine-2,6-dione (37 mg, 114.50 μmol) in DMSO (1.0 mL) were added Pd(dppf)Cl₂ (8.38 mg, 11.45 μmol), CuI (2.18 mg, 11.45 μmol) and TEA (115.86 mg, 1.15 mmol). The reaction mixture was stirred at 90 [symbol] for 2 h, before it was purified by reverse phase chromatography (MeCN:H₂O:TFA=90:10:0.5) to afford the title compound (20 mg, yield: 31%) as a white solid. MS (ESI) m/z=557.6 [M+H]⁺.

### Step 6. Synthesis of 3-(6-(2-(trans-3-(3-cyclopropyl-4-(quinoxalin-2-yl)-1H-pyrazol-1-yl)cyclobutyl)ethyl)-1-oxoisoindolin-2-yl)piperidine-2,6-dione

To a solution of 3-(6-((trans-3-(3-cyclopropyl-4-(quinoxalin-2-yl)-1H-pyrazol-1-yl)cyclobutyl)ethynyl)-1-oxoisoindolin-2-yl)piperidine-2,6-dione (20 mg, 35.93 μmol) in isopropanol (1.0 mL) and EtOAc (1.0 mL) was added Pd/C (20 mg). The reaction mixture was stirred at rt for 1 h under H₂, before it was filtered. To the filtrate was added MnO₂ (50 mg, 575.13 μmol). After the resulting mixture was stirred at rt for 0.5 h, it was filtered, and concentrated. The residue was purified by reverse phase chromatography (MeCN:H₂O:TFA=80:20:0.5), prep-TLC (DCM:MeOH=20:1), and prep-HPLC (MeCN, H₂O containing 0.5% TFA) to afford the title compound (1.19 mg, yield: 6%) as a yellow solid. MS (ESI) m/z=561.8 [M+H]⁺.

### Example 85. 3-(6-((Trans-3-(3-cyclopropyl-4-(quinoxalin-2-yl)-1H-pyrazol-1-yl)cyclobutyl)ethynyl)-1-oxoisoindolin-2-yl)piperidine-2,6-dione (GS-726)

### Step 1. Synthesis of 3-cyclopropyl-4-iodo-1H-pyrazole

To a solution of tert-butyl 3-cyclopropyl-4-iodo-1H-pyrazole-1-carboxylate (550 mg, 1.65 mmol) in DCM (1.0 mL) was added TFA (187.68 mg, 1.65 mmol, 2.0 mL). The reaction was stirred at rt for 0.5 h, before it was concentrated. Sat. NaHCO₃ solution was added to adjust pH to 6˜7. The mixture was extracted with EtOAc (20 mL×3). The combined organic layers were washed with brine, dried and filtered. The filtrate was concentrated to afford the title compound (370 mg, yield: 96%) as a light oil.

### Step 2. Synthesis of 3-cyclopropyl-4-iodo-1-(tetrahydro-2H-pyran-2-yl)-1H-pyrazole

To a solution of 3-cyclopropyl-4-iodo-1H-pyrazole (370 mg, 1.58 mmol) in DCM (5.0 mL) were added PTS (89.84 mg, 521.71 μmol) and DHP (359.05 mg, 4.27 mmol). The mixture was stirred at 60 [symbol] for 3 h, before it was cooled to rt. Sat. NaHCO₃ solution was added to adjust pH to 6˜7. The mixture was extracted with EtOAc (20 mL×2). The combined organic layers were washed with brine, dried over Na₂SO₄, filtered and concentrated. The residue was purified by silica gel chromatography (EtOAc:petroleum ether=1:4) to afford the title compound (430 mg, yield: 85%) as a light yellow oil. MS (ESI) m/z=319.3 [M+H]⁺.

### Step 3. Synthesis of 3′-cyclopropyl-1′-(tetrahydro-2H-pyran-2-yl)-1′H-1,4′-bipyrazole

To a solution of 3-cyclopropyl-4-iodo-1-(tetrahydro-2H-pyran-2-yl)-1H-pyrazole (2.10 g, 6.61 mmol) and 1H-pyrazole (300 mg, 4.41 mmol) in MeCN (15 mL) were added 2-hydroxybenzaldehyde oxime (120.87 mg, 881.35 μmol), Cu₂O(31.53 mg, 220.34 μmol) and Cs₂CO₃ (2.87 g, 8.81 mmol). The mixture was stirred at 100 [symbol] for 10 h under microwave irradiation, before it was filtered. The filtrate was concentrated. And the resulting residue was purified by silica gel chromatography (EtOAc:petroleum ether=3:7) to afford the title compound (130 mg, yield: 11%) as a white solid. MS (ESI) m/z=259.5 [M+H]⁺.

### Step 4. Synthesis of 3′-cyclopropyl-′H-1,4′-bipyrazole

To a solution of 3′-cyclopropyl-1′-(tetrahydro-2H-pyran-2-yl)-1′H-1,4′-bipyrazole (130 mg, 503.26 μmol) in DCM (1.0 mL) was added TFA (57.38 mg, 503.26 μmol, 1.0 mL). The mixture was stirred at rt for 0.5 h, before it was concentrated. Sat. NaHCO₃ solution was added to adjust pH to 6˜7. The mixture was extracted with EtOAc (20 mL×2). The combined organic layers were washed with brine, dried over Na₂SO₄, filtered and concentrated to afford the title compound (80 mg, yield: 91%) as a light oil. MS (ESI) m/z=175.4 [M+H]⁺.

### Step 5 to 9. Synthesis of 3-(6-((trans-3-(3-cyclopropyl-4-(quinoxalin-2-yl)-1H-pyrazol-1-yl)cyclobutyl)ethynyl)-1-oxoisoindolin-2-yl)piperidine-2,6-dione

GS-726 was synthesized following the procedures for steps 3 to 7 of GS-714 (TFA salt, 8.8 mg, yield: 0.1% over 5 steps) as a yellow solid. MS (ESI) m/z=514.6 [M+H]⁺.

### Example 86. 5-(((Trans-3-(2H-indazol-2-yl)cyclobutyl)methyl)amino)-2-(2,6-dioxopiperidin-3-yl)isoindoline-1,3-dione (GS-727)

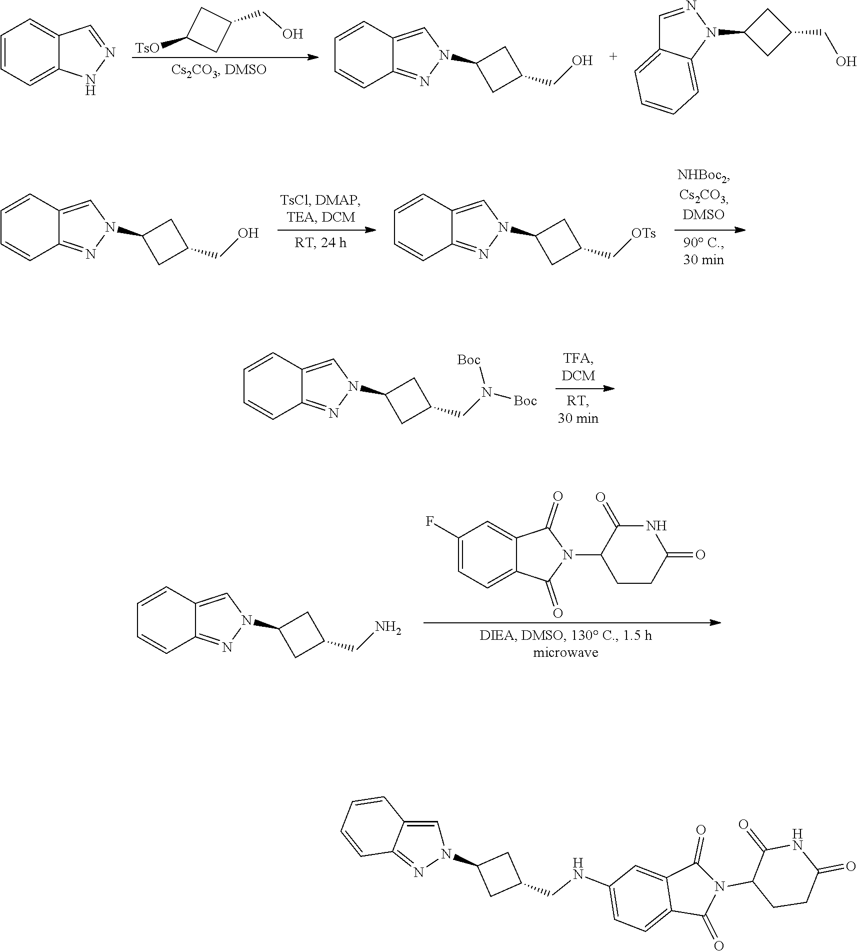

### Step 1. Synthesis of (trans-3-(2H-indazol-2-yl)cyclobutyl)methanol and (trans-3-(1H-indazol-1-yl)cyclobutyl)methanol

A mixture of 1H-indazole (500 mg, 4.23 mmol), cis-3-(hydroxymethyl)cyclobutyl 4-methylbenzenesulfonate (1.08 g, 4.23 mmol) and Cs₂CO₃ (4.12 g, 12.69 mmol) in DMSO (15 mL) was stirred at 90 [symbol] for 3 h, before it was diluted with EtOAc (50 mL) and washed with water and brine. The organic layer was concentrated, and the residue was purified by prep-TLC to afford the title compounds.

(Trans-3-(2H-indazol-2-yl)cyclobutyl)methanol (320.1 mg, yield: 37%) was obtained as a pale solid. MS (ESI) m/z=203.5 [M+H]⁺.

(Trans-3-(1H-indazol-1-yl)cyclobutyl)methanol (180.0 mg, yield: 21%) was obtained as a pale solid. MS (ESI) m/z=203.5 [M+H]⁺.

### Step 2 to 5. Synthesis of 5-(((trans-3-(2H-indazol-2-yl)cyclobutyl)methyl)amino)-2-(2,6-dioxopiperidin-3-yl)isoindoline-1,3-dione

GS-727 was synthesized following the procedures for steps 4 to 7 of GS-714 (96.0 mg, yield: 18% over 4 steps) as a yellow solid. MS (ESI) m/z=458.5 [M+H]⁺.

### Example 87. 5-(((Trans-3-(1H-indazol-1-yl)cyclobutyl)methyl)amino)-2-(2,6-dioxopiperidin-3-yl)isoindoline-1,3-dione (GS-728)

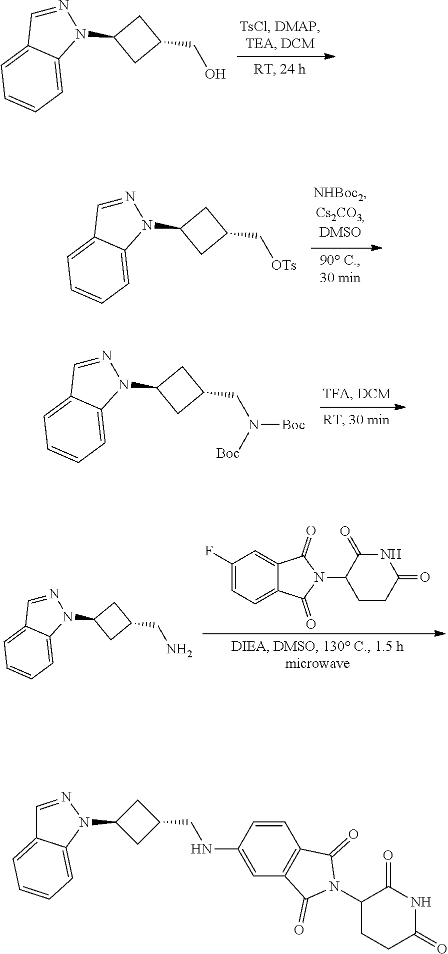 [SMILES: OC[C@H]1C[C@H](n2ncc3ccccc32)C1 ; Cc1ccc(S(=O)(=O)OC[C@H]2C[C@H](n3ncc4ccccc43)C2)cc1 ; CC(C)(C)OC(=O)N(C[C@H]1C[C@H](n2ncc3ccccc32)C1)C(=O)OC(C)(C)C ; NC[C@H]1C[C@H](n2ncc3ccccc32)C1 ; O=C1CCC(N2C(=O)c3ccc(F)cc3C2=O)C(=O)N1 ; O=C1CCC(N2C(=O)c3ccc(NC[C@H]4C[C@H](n5ncc6ccccc65)C4)cc3C2=O)C(=O)N1]

GS-728 was synthesized following the procedures for steps 4 to 7 of GS-714 (116.0 mg, yield: 29% over 4 steps) as a yellow solid. MS (ESI) m/z=458.5 [M+H]⁺.

### Example 88. 5-(((Trans-3-(3-cyclopropyl-4-(1-methyl-1H-pyrazolo[3,4-b]pyridin-6-yl)-1H-pyrazol-1-yl)cyclobutyl)methyl)amino)-2-(2,6-dioxopiperidin-3-yl)isoindoline-1,3-dione (GS-729)

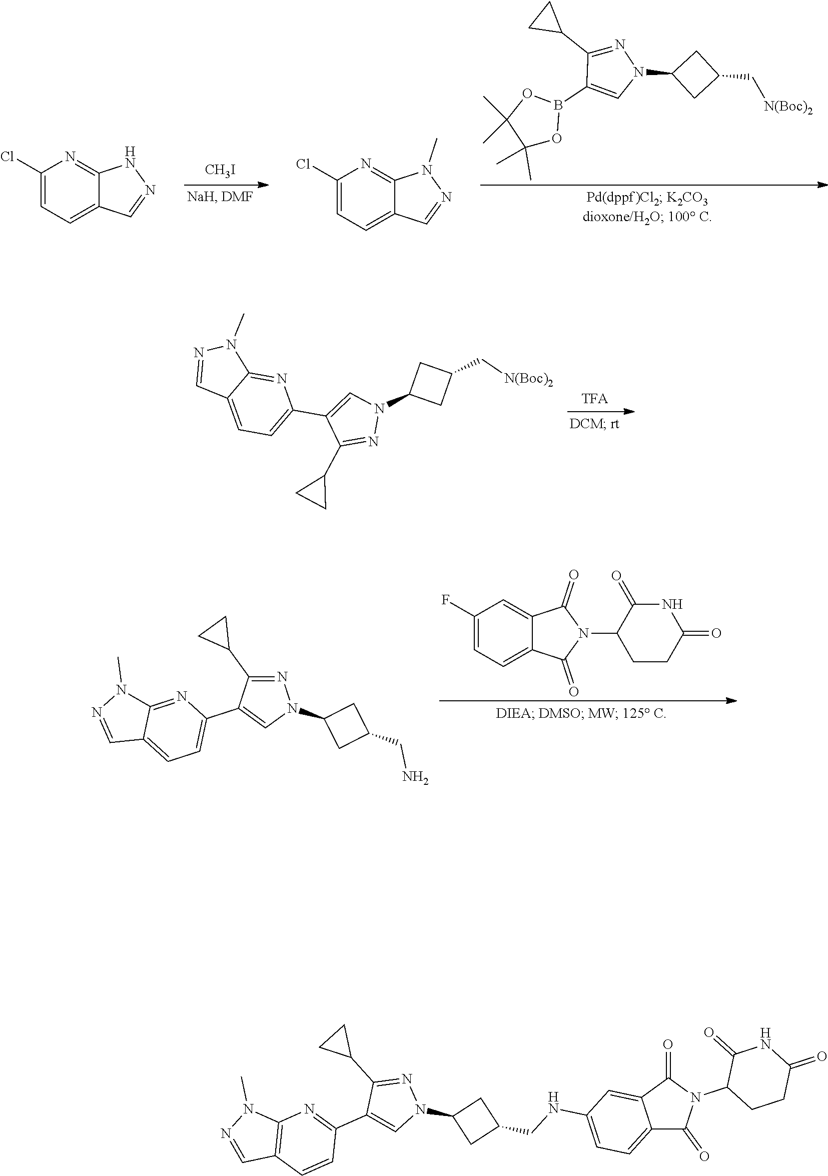

### Step 1. Synthesis of 6-chloro-1-methyl-1H-pyrazolo[3,4-b]pyridine

To a solution of 6-chloro-1H-pyrazolo [3,4-b]pyridine (1.0 g, 6.51 mmol) in DMF (10 mL) was added NaH (60% in mineral oil, 390.24 mg, 9.76 mmol) at 0 [symbol] . The mixture was stirred at this temperature for 10 min. Iodomethane (1.85 g, 13.02 mmol) was added. The mixture was stirred at rt for 1 h. Water (10 mL) and anhydrous Na₂SO₄ were added subsequentially. The suspension was filtered and the filtrate was concentrated to provide the title compound (960.0 mg, yield: 89%) as a colorless solid. MS (ESI) m/z=168.5[M+H]⁺.

### Step 2. Synthesis of tert-butyl (tert-butoxycarbonyl)((trans-3-(3-cyclopropyl-4-(1-methyl-1H-pyrazolo[3,4-b]pyridin-6-yl)-1H-pyrazol-1-yl)cyclobutyl)methyl)carbamate

To a stirred solution of 6-chloro-1-methyl-1H-pyrazolo[3,4-b]pyridine (65.90 mg, 393.23 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (305.2 mg, 589.8 μmol) in 1,4-dioxane (15 mL) and water (1.5 mL) were added Pd(dppf)Cl₂ (28.7 mg, 39.3 μmol) and K₂CO₃ (162.8 mg, 1.18 mmol). The mixture was purged with Argon 3 times and stirred at 100° C. overnight. Water (50 mL) was added. The mixture was extracted with EtOAc (50 mL×3). The combined organic phase was washed with brine, dried over Na₂SO₄ and concentrated. The residue was purified by silica gel chromatography to afford the title compound (34 mg, yield: 17%) as a solid. MS (ESI) m/z=523.8 [M+H]⁺.

### Step 3. Synthesis of (trans-3-(3-cyclopropyl-4-(1-methyl-1H-pyrazolo[3,4-b]pyridin-6-yl)-1H-pyrazol-1-yl)cyclobutyl)methanamine

To a stirred solution of tert-butyl (tert-butoxycarbonyl)((trans-3-(3-cyclopropyl-4-(1-methyl-1H-pyrazolo[3,4-b]pyridin-6-yl)-1H-pyrazol-1-yl)cyclobutyl)methyl)carbamate (270.0 mg, 672.48 μmol) in DCM (10 mL) was added TFA (2.5 mL). The mixture was stirred at rt for 1.5 h, before it was concentrated to afford the title compound (TFA salt, 20 mg, yield: 96%). MS (ESI) m/z=323.5 [M+H]⁺.

### Step 4. Synthesis of 5-((trans-3-(3-cyclopropyl-4-(1-methyl-1H-pyrazolo[3,4-b]pyridin-6-yl)-1-pyrazol-1-yl)cyclobutyl)methyl)amino)-2-(2,6-dioxopiperidin-3-yl)isoindoline-1,3-dione

To a solution of (trans-3-(3-cyclopropyl-4-(1-methyl-1H-pyrazolo[3,4-b]pyridin-6-yl)-1H-pyrazol-1-yl)cyclobutyl)methanamine (20 mg, 62.03 μmol) in DMSO (2.5 mL) were added 2-(2,6-dioxopiperidin-3-yl)-5-fluoroisoindoline-1,3-dione (22.2 mg, 80.6 μmol) and DIEA (80.0 mg, 620.3 μmol). The mixture was stirred at 125 [symbol] for 2.5 h, before it was purified by reverse phase chromatography to afford the title compound (13.0 mg, yield: 36%) as a yellow solid. MS (ESI) m/z=579.6 [M+H]⁺.

### Example 89. 5-(((Trans-3-(3-cyclopropyl-4-(1-isopropyl-1H-pyrazolo[3,4-b]pyridin-6-yl)-1H-pyrazol-1-yl)cyclobutyl)methyl)amino)-2-(2,6-dioxopiperidin-3-yl)isoindoline-1,3-dione (GS-730)

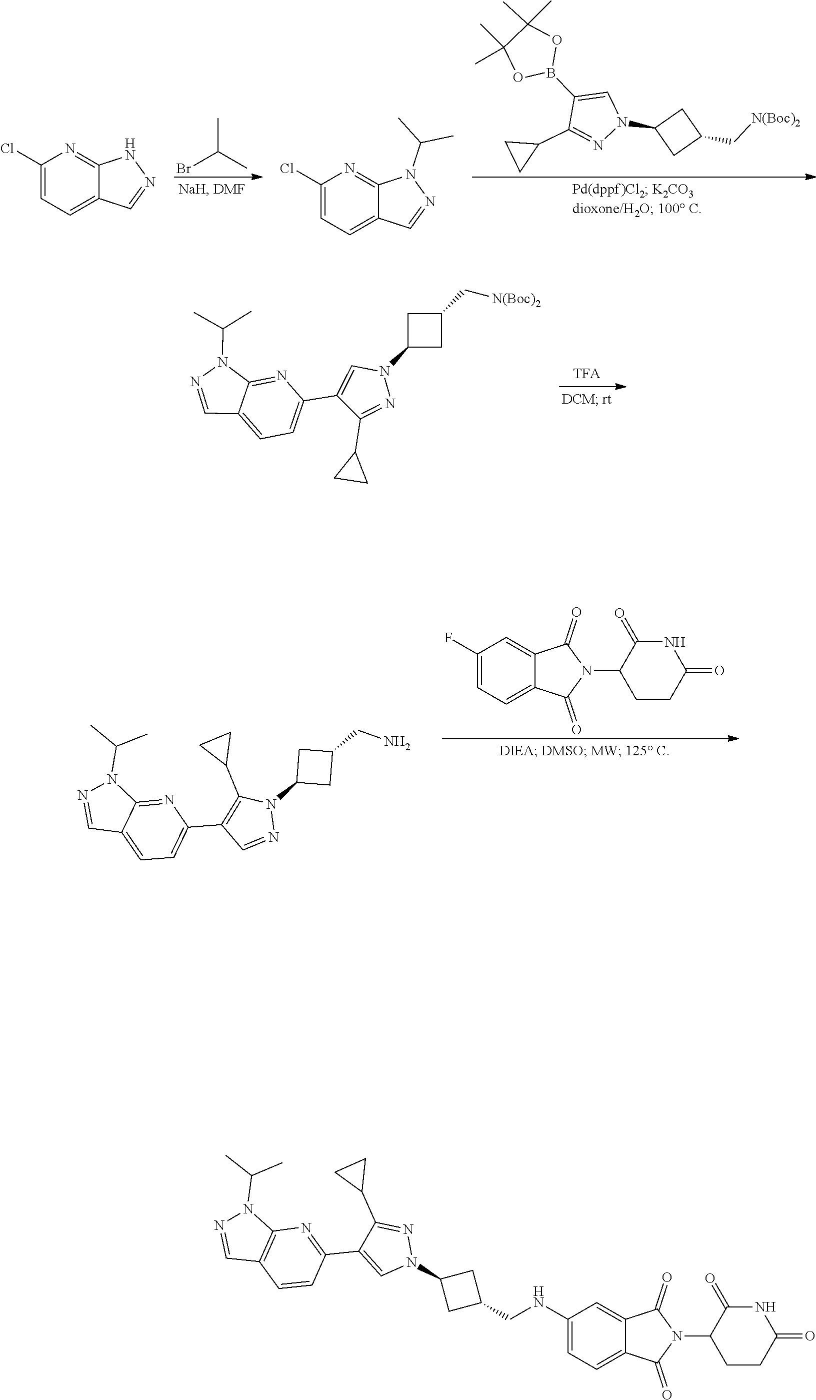

### Step 1. Synthesis of 6-chloro-1-isopropyl-1H-pyrazolo[3,4-b]pyridine

To a solution of 6-chloro-1H-pyrazolo[3,4-b]pyridine (1.0 g, 6.55 mmol) in DMF (5 mL) was added NaH (235.9 mg, 9.83 mmol) at 0 [symbol] . The mixture was stirred at 0 [symbol] for 10 min before 2-bromopropane (800.8 mg, 6.51 mmol) was added. The mixture was then stirred at 0 [symbol] overnight. The mixture was diluted with H₂O (10 mL) and extracted with EtOAc (50 mL×3). The combined organic layers were washed with brine, dried over Na₂SO₄ and concentrated. The residue was purified by silica gel chromatography to afford the title compound (680 mg, yield: 63%) as a colorless solid. MS (ESI) m/z=196.2 [M+H]⁺.

### Step 2. Synthesis of tert-butyl (tert-butoxycarbonyl)((trans-3-(3-cyclopropyl-4-(1-methyl-1H-pyrazolo[3,4-b]pyridin-6-yl)-1H-pyrazol-1-yl)cyclobutyl)methyl)carbamate

To a stirred solution of 6-chloro-1-isopropyl-1H-pyrazolo[3,4-b]pyridine (60.0 mg, 306.67 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (238.0 mg, 460.0 μmol) in 1,4-dioxane (10 mL) and H₂O (1 mL) were added Pd(dppf)Cl₂ (22.4 mg, 30.6 μmol) and K₂CO₃ (126.9 mg, 920.0 μmol). The mixture was purged with Ar. The mixture was stirred at 100 [symbol] overnight. H₂O (50 mL) was added, and the mixture was extracted with EtOAc (50 mL×3). The combined organic layers were washed with brine, dried over Na₂SO₄ and concentrated. The residue was purified by silica gel chromatography to afford the title compound (45 mg, yield: 27%) as a solid. MS (ESI) m/z=551.8 [M+H]⁺.

### Step 3. Synthesis of (trans-3-(5-cyclopropyl-4-(1-methyl-1H-pyrazolo[3,4-b]pyridin-6-yl)-1H-pyrazol-1-yl)cyclobutyl)methanamine

To a stirred solution of tert-butyl (tert-butoxycarbonyl)((trans-3-(3-cyclopropyl-4-(1-methyl-1H-pyrazolo[3,4-b]pyridin-6-yl)-1H-pyrazol-1-yl)cyclobutyl)methyl)carbamate (45 mg, 81.7 μmol) in DCM (10 mL) was added TFA. The mixture was stirred at rt for 1.5 h, before it was concentrated to afford the title compound (TFA salt, 25 mg, yield: 87%). MS (ESI) m/z=351.5 [M+H]⁺.

### Step 4. Synthesis of 5-(((trans-3-(3-cyclopropyl-4-(1-methyl-1H-pyrazolo[3,4-b]pyridin-6-yl)-1H-pyrazol-1-yl)cyclobutyl)methyl)amino)-2-(2,6-dioxopiperidin-3-yl)isoindoline-1,3-dione

To a solution of (trans-3-(5-cyclopropyl-4-(1-methyl-1H-pyrazolo[3,4-b]pyridin-6-yl)-1H-pyrazol-1-yl)cyclobutyl)methanamine (25.0 mg, 71.3 μmol) in DMSO (2.5 mL) were added 2-(2,6-dioxopiperidin-3-yl)-5-fluoroisoindoline-1,3-dione (25.6 mg, 92.7 μmol) and DIEA (92.0 mg, 713.3 μmol). The mixture was stirred at 125 [symbol] for 2.5 h, before it was purified by reverse phase chromatography to afford the title compound (15 mg, yield: 35%) as a yellow solid. MS (ESI) m/z=607.7 [M+H]⁺.

### Example 90. 3-(5-(((Trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-731)

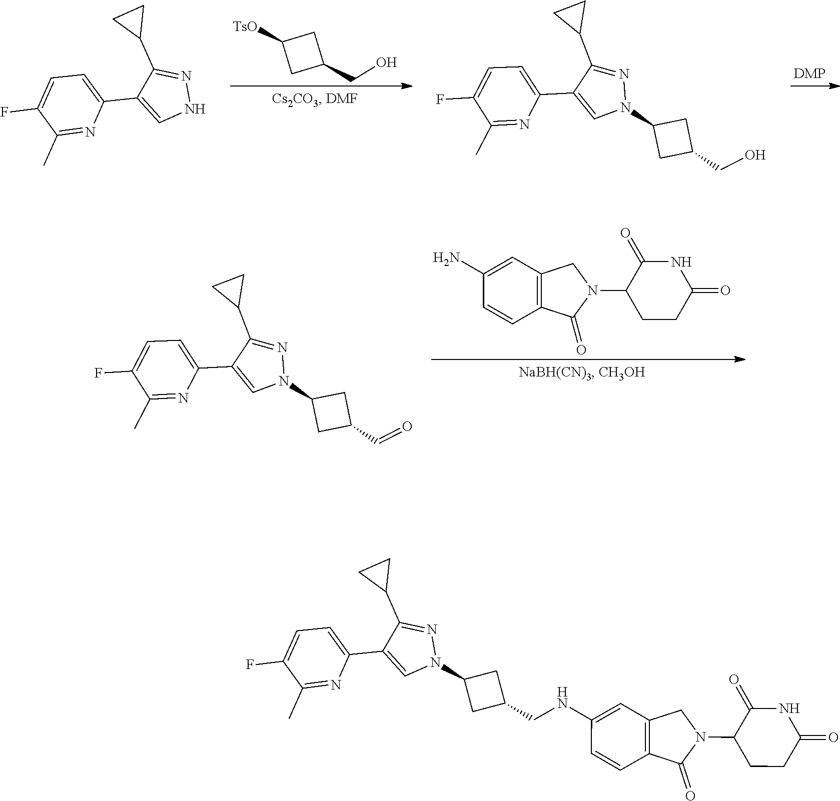 [SMILES: Cc1nc(-c2c[nH]nc2C2CC2)ccc1F ; Cc1ccc(S(=O)(=O)O[C@H]2C[C@@H](CO)C2)cc1 ; Cc1nc(-c2cn([C@H]3C[C@H](CO)C3)nc2C2CC2)ccc1F ; Cc1nc(-c2cn([C@H]3C[C@H](C=O)C3)nc2C2CC2)ccc1F ; Nc1ccc2c(c1)CN(C1CCC(=O)NC1=O)C2=O ; Cc1nc(-c2cn([C@H]3C[C@H](CNc4ccc5c(c4)CN(C4CCC(=O)NC4=O)C5=O)C3)nc2C2CC2)ccc1F]

### Step 1. Synthesis of (trans-3-(3-cyclopropyl-4-(5-fluoro-6-methylpyridin-2-yl)-1H-pyrazol-1-yl)cyclobutyl)methanol

To a solution of 6-(3-cyclopropyl-1H-pyrazol-4-yl)-3-fluoro-2-methylpyridine (2 g, 9.21 mmol) in DMF (10 mL) were added cis-3-(hydroxymethyl)cyclobutyl 4-methylbenzenesulfonate (2.36 g, 9.21 mmol) and Cs₂CO₃ (5.98 g, 18.41 mmol). The mixture was stirred at 90 [symbol] for 16 h, before it was purified by reverse phase chromatography (water:MeOH=1:1) to afford the title compound (1.2 g, yield: 43%) as a yellow oil. MS (ESI) m/z=302.3 [M+H]⁺.

### Step 2. Synthesis of trans-3-(3-cyclopropyl-4-(5-fluoro-6-methylpyridin-2-yl)-1H-pyrazol-1-yl)cyclobutane-1-carbaldehyde

To a solution of (trans-3-(3-cyclopropyl-4-(5-fluoro-6-methylpyridin-2-yl)-1H-pyrazol-1-yl)cyclobutyl)methanol (300 mg, 995.49 μmol) in DCM (10 mL) was added Dess-Martin periodinane (422.23 mg, 995.49 μmol). The mixture was stirred at it for 2 h, before it was filtered. The filtration was washed with water (20 mL), and concentrated to afford the title compound (290 mg, yield: 97%) as a yellow oil, which was used directly in the next step. MS (ESI) m/z=300.2 [M+H]⁺.

### Step 3. Synthesis of 3-(5-(((trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione

To a solution of trans-3-(3-cyclopropyl-4-(5-fluoro-6-methylpyridin-2-yl)-1H-pyrazol-1-yl)cyclobutane-1-carbaldehyde (50 mg, 167.03 μmol) in methanol (2 mL) were added 3-(5-amino-1-oxo-isoindolin-2-yl)piperidine-2,6-dione (43.30 mg, 167.03 μmol), NaBH(CN)₃ (51.78 mg, 835.17 μmol) and a drop of HOAc. The mixture was stirred at rt for 30 min. The residue was purified by reverse phase chromatography (water:MeOH=1:1) to afford the title compound (10 mg, yield: 11%) as a white solid. MS (ESI) m/z=543.3 [M+H]⁺.

### Example 91. 3-(6-(((Trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-732)

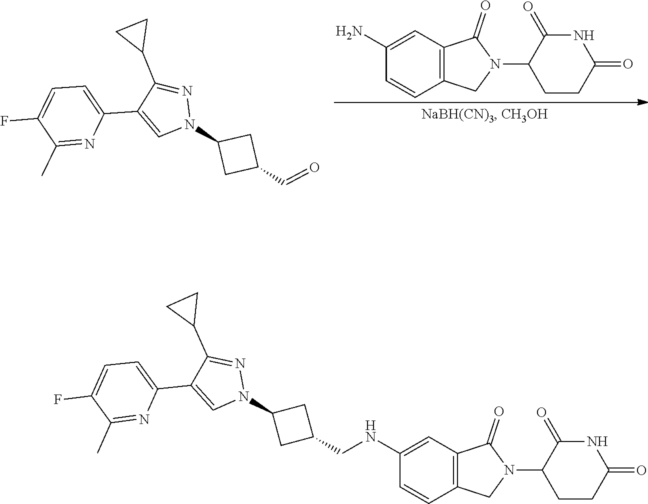 [SMILES: Cc1nc(-c2cn([C@H]3C[C@H](C=O)C3)nc2C2CC2)ccc1F ; Nc1ccc2c(c1)C(=O)N(C1CCC(=O)NC1=O)C2 ; Cc1nc(-c2cn([C@H]3C[C@H](CNc4ccc5c(c4)C(=O)N(C4CCC(=O)NC4=O)C5)C3)nc2C2CC2)ccc1F]

GS-732 was synthesized following the similar procedure as described for step 3 of GS-731 (9 mg, yield: 10%) as a white solid. MS (ESI) m/z=543.3 [M+H]⁺.

### Example 92. 5-(((Trans-3-(3-cyclopropyl-4-(7-fluoro-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-733)

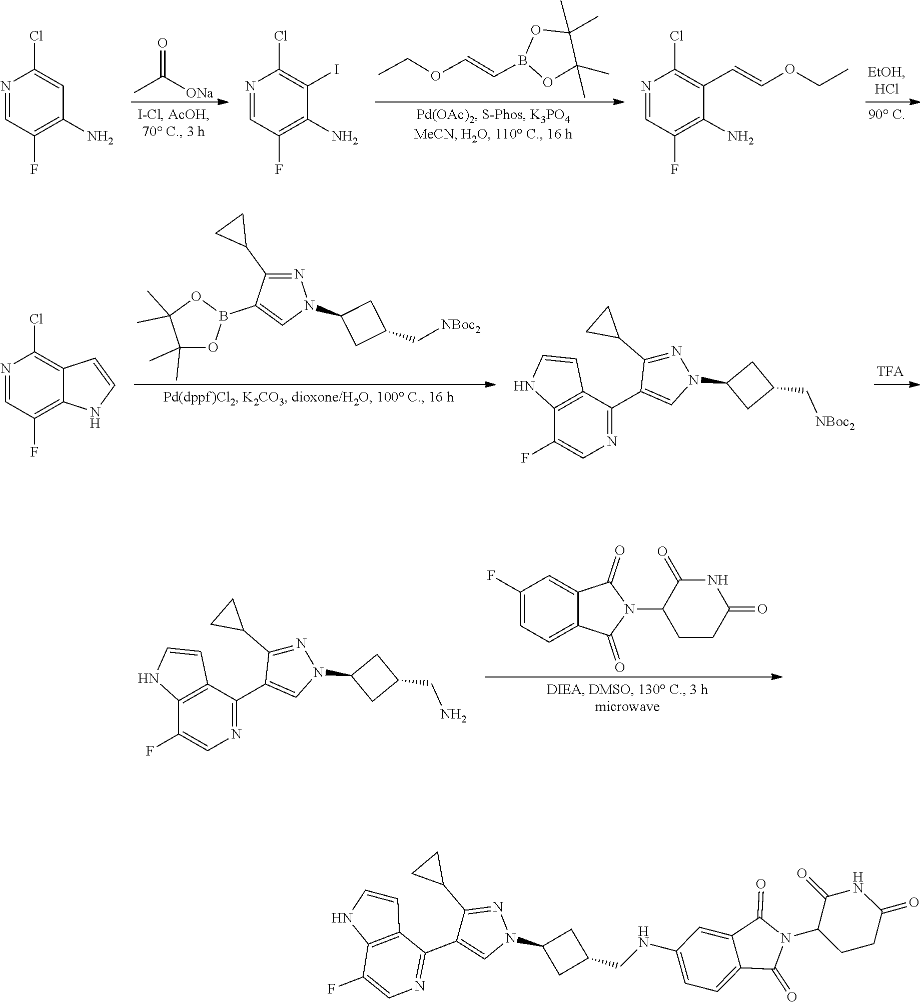

### Step 1. Synthesis of 2-chloro-5-fluoro-3-iodopyridin-4-amine

A solution of 2-chloro-5-fluoropyridin-4-amine (2.00 g, 13.7 mmol), sodium acetate (2.25 g, 27.4 mmol) and iodine monochloride (4.45 g, 27.4 mmol) in acetic acid (20 mL) was stirred at 70 [symbol] for 3 h, before it was cooled to rt and concentrated. The residue was diluted with water and basified with aq. NaHCO₃ solution. The mixture was extracted with EtOAc (50 mL×2). The combined organic phase was washed with brine, dried over Na₂SO₄, filtered and concentrated. The residue was purified by silica gel chromatography (petroleum ether:EtOAc=1:1) to afford the title compound (2.50 g, yield: 71%) as a light brown solid. MS (ESI) m/z=272.9 [M+H]⁺.

### Step 2. Synthesis of 2-chloro-3-(2-ethoxyvinyl)-S-fluoropyridin-4-amine

A stirred solution of 2-chloro-5-fluoro-3-iodopyridin-4-amine (2.00 g, 7.34 mmol), 2-(2-ethoxyvinyl)-4,4,5,5-tetramethyl-1,3,2-dioxaborolane (1.74 g, 8.81 mmol), potassium phosphate (3.12 g, 14.7 mmol), palladium acetate (50 mg, 0.220 mmol) and S-phos (226 mg, 0.550 mmol) in acetonitrile/water (3:2, 50 mL) was stirred in a sealed tube at 110 [symbol] for 16 h. Upon completion, the reaction mixture was cooled to rt, and filtered through Celite. The filter cake was washed with EtOAc (100 mL). The filtrate was washed with water and brine and concentrated. The residue was purified by silica gel chromatography (petroleum ether:EtOAc=5:1) to afford the title compound (1.30 g, yield: 82%) as a yellowish solid. MS (ESI) m/z=217.1 [M+H]⁺.

### Step 3. Synthesis of 4-chloro-7-fluoro-1H-pyrrolo[3,2-c]pyridine

A solution of 2-chloro-3-(2-ethoxyvinyl)-5-fluoropyridin-4-amine (1.30 g, 6.02 mmol) in ethanol (18 mL)/conc. HCl (4 mL) was stirred at 90 [symbol] for 2 h. After the reaction was cooled to rt, it was basified with aqueous NaHCO₃ solution, and extracted with EtOAc (50 mL×3). The combined organic layers were dried over Na₂SO₄, filtered and concentrated. The residue was purified by silica gel chromatography (petroleum ether:EtOAc=4:1) to afford the title compound (752 mg, yield: 74%) as an off-white solid. ¹H NMR (400 MHz, DMSO-d₆) δ 12.59 (s, 1H), 8.01 (d, J=2.4 Hz, 1H), 7.67 (s, 1H), 6.65 (d, J=1.2 Hz, 1H). MS (ESI) m/z=171.1 [M+H]⁺.

### Step 4. Synthesis of tert-butyl (tert-butoxycarbonyl)((trans-3-(3-cyclopropyl-4-(7-fluoro-1H-pyrrolo[3,2-c]pyridin-4-yl)-1H-pyrazol-1-yl)cyclobutyl)methyl)carbamate

To a solution of 4-chloro-7-fluoro-1H-pyrrolo[3,2-c]pyridine (100 mg, 586.27 μmol) and trans-tert-butyl N-tert-butoxycarbonyl-N-[[3-[3-cyclopropyl-4-(4,4,5,5-tetramethyl-1,3,2-dioxaborolan-2-yl)pyrazol-1-yl]cyclobutyl]methyl]carbamate (455.06 mg, 879.40 μmol) in water (0.1 mL) and 1,4-dioxane (5.0 mL) were added Pd(dppf)Cl₂ (42.90 mg, 58.63 μmol) and K₂CO₃ (243.08 mg, 1.76 mmol). The reaction mixture was stirred at 100 [symbol] for 16 h, before it was poured into ice/water, and extracted with EtOAc (20 mL×2). The combined organic layers were washed with brine, dried with Na₂SO₄, filtered, and concentrated. The residue was purified by prep-TLC (EtOAc:petroleum ether=1:1) to afford the title compound (47.3 mg, yield: 15%) as an oil. MS (ESI) m/z=526.6 [M+H]⁺.

### Step 5. Synthesis of (trans-3-(3-cyclopropyl-4-(7-fluoro-1H-pyrrolo[3,2-c]pyridin-4-yl)-1H-pyrazol-1-yl)cyclobutyl)methanamine

To a solution of tert-butyl (tert-butoxycarbonyl)((trans-3-(3-cyclopropyl-4-(7-fluoro-1H-pyrrolo[3,2-c]pyridin-4-yl)-1H-pyrazol-1-yl)cyclobutyl)methyl)carbamate (47.3 mg, 89.99 μmol) in DCM (1.5 mL) was added TFA (10.26 mg, 89.99 μmol, 2.0 mL). The reaction mixture was stirred at rt for 1 h, before it was concentrated to afford the title compound (TFA salt, 30.7 mg, yield: 19%) as a brown oil. MS (ESI) m/z=326.3 [M+H]⁺.

### Step 6. Synthesis of 5-(((trans-3-(3-cyclopropyl-4-(7-fluoro-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(7-fluoro-1H-pyrrolo[3,2-c]pyridin-4-yl)-1H-pyrazol-1-yl)cyclobutyl)methanamine (TFA salt, 30.7 mg, 39.28 μmol) and 2-(2,6-dioxo-3-piperidyl)-5-fluoro-isoindoline-1,3-dione (13.02 mg, 47.14 μmol) in DMSO (0.5 mL) was added DIPEA (101.54 mg, 785.69 μmol, 129.85 μL). The mixture was stirred at 130° C. for 3 h under microwave irradiation, before it was purified by reverse phase chromatography (MeOH:H₂O:TFA=60:40:0.5) to afford the title compound (3.86 mg, yield: 17%) as a yellow solid. MS (ESI) m/z=582.7 [M+H]⁺.

### Example 93. 5-(((Trans-3-(4-benzyl-3-cyclopropyl-1H-pyrazol-1-yl)cyclobutyl)methyl)amino)-2-(2,6-dioxopiperidin-3-yl)isoindoline-1,3-dione (GS-735)

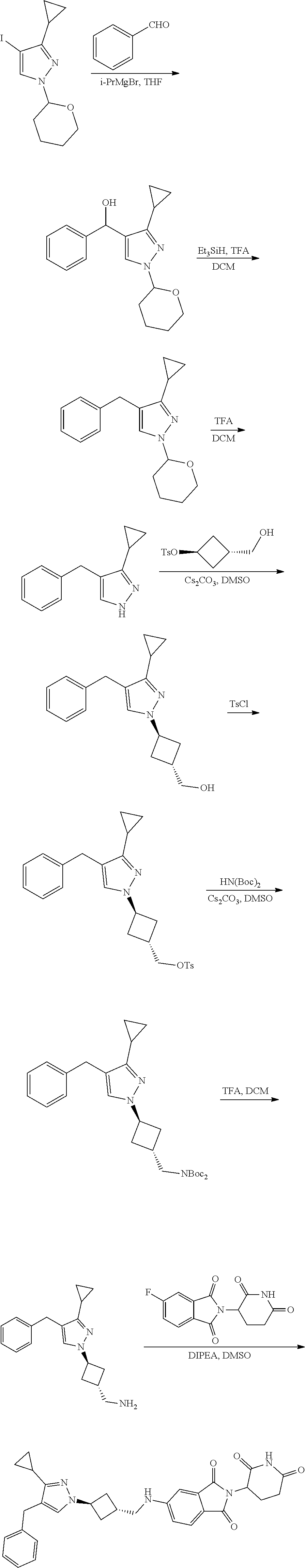

### Step 1. Synthesis of (3-cyclopropyl-1-tetrahydropyran-2-yl-pyrazol-4-y)-phenyl-methanol

To a solution of 3-cyclopropyl-4-iodo-1-tetrahydropyran-2-yl-pyrazole (2.0 g, 6.29 mmol) in THF (40 mL) was added i-PrMgBr (7.54 mmol, 2.7 mL) at 0 [symbol] . The resulting mixture was stirred at this temperature for 0.5 h before benzaldehyde (667.11 mg, 6.29 mmol) was added. The mixture was warmed to rt, and stirred for another 1 h, before it was quenched with aq. NH₄Cl solution (40 mL) and extracted with EtOAc (40 mL×3). The combined organic phase was dried over Na₂SO₄, filtered and concentrated. The residue was purified by silica gel chromatography (petroleum ether:EtOAc=5:1 to 1:1) to afford the title compound (1.5 g, yield: 80%). MS (ESI) m/z=299.6 [M+H]⁺.

### Step 2 to 3. Synthesis of 4-benzyl-3-cyclopropyl-1H-pyrazole

To a solution of (3-cyclopropyl-1-tetrahydropyran-2-yl-pyrazol-4-yl)-phenyl-methanol (1.5 g, 5.03 mmol) in DCM (20 mL) were added TFA (2.87 g, 25.14 mmol) and Et₃SiH (2.92 g, 25.14 mmol) at rt. The mixture was stirred at 30 [symbol] for 24 h. More TFA(10 mL) was added. The mixture was stirred at 30 [symbol] for another 4 h, before it was concentrated. Aq. NaHCO₃ solution (50 mL) was added. The mixture was extracted with EtOAc (50 mL×3). The combined organic phase was concentrated and purified by silica gel chromatography (petroleum ether:EtOAc=5:1 to 1:1) to afford the title compound (740 mg, yield: 74%) as a light-yellow oil. MS (ESI) m/z=199.5 [M+H]⁺.

### Step 4 to 8. Synthesis of (trans-3-(4-benzyl-3-cyclopropyl-1H-pyrazol-1-yl)cyclobutyl)methanol

GS-735 was synthesized following the procedures for steps 3 to 7 of GS-714 (120 mg, yield: 6% over 5 steps) as a yellow solid. MS (ESI) m/z=538.7 [M+H]⁺.

### Example 94. 5-(((Trans-3-(3-cyclopropyl-4-(1H-indazol-1-yl)-1H-pyrazol-1-yl)cyclobutyl)methyl)amino)-2-(2,6-dioxopiperidin-3-yl)isoindoline-1,3-dione (GS-736)

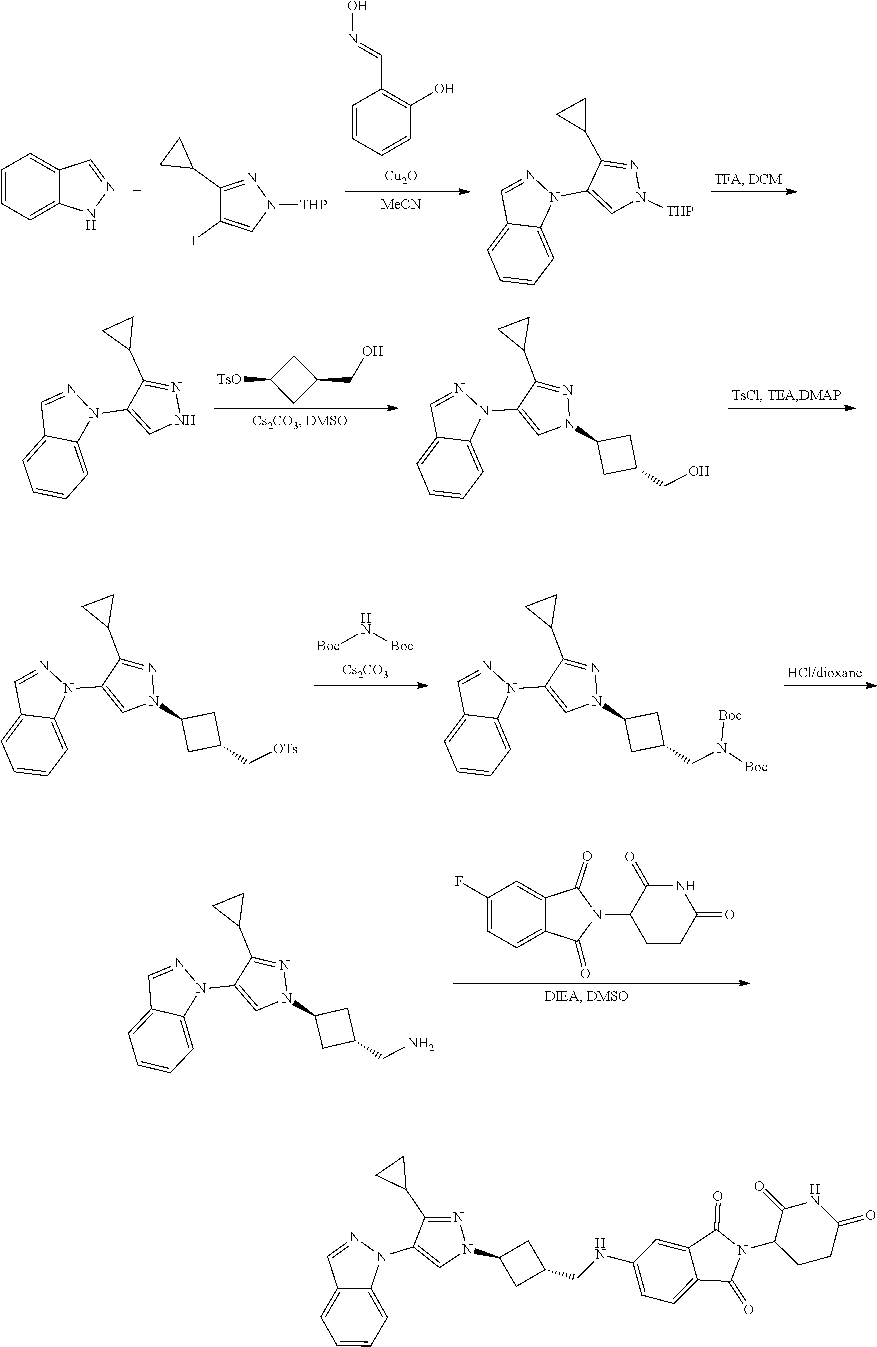

### Step 1. Synthesis of 1-(3-cyclopropyl-1-(tetrahydro-2H-pyran-2-yl)-1H-pyrazol-4-yl)-1H-indazole

To a solution of 1H-indazole (500 mg, 4.23 mmol) and 3-cyclopropyl-4-iodo-1-tetrahydropyran-2-yl-pyrazole (1.35 g, 4.23 mmol) in ACN (10 mL) were added Cu₂O (609.47 mg, 4.23 mmol) and 2-hydroxybenzaldehyde oxime (580.42 mg, 4.23 mmol). The mixture was heated at 100 [symbol] under N₂ for 10 h under microwave irradiation, before it was concentrated. The residue was purified by silica gel chromatography (petroleum ether:EtOAc=3:1) to afford the title compound (150.5 mg, yield: 11%) as a yellow oil. MS (ESI) m/z=309.6 [M+H]⁺.

### Step 2. Synthesis of 1-(3-cyclopropyl-1H-pyrazol-4-yl)-1H-indazole

To a stirred solution of 1-(3-cyclopropyl-1-(tetrahydro-2H-pyran-2-yl)-1H-pyrazol-4-yl)-1H-indazole (150 mg, 481.55 μmol) in DCM (2 mL) was added TFA (1.10 g, 9.63 mmol). The mixture was stirred at 20 [symbol] for 1 h, before it was concentrated. The residue was purified by prep-TLC to afford the title compound (77.6 mg, yield: 71%) as a colorless oil. MS (ESI) m/z=225.5 [M+H]⁺.

### Step 3. Synthesis of (trans-3-(3-cyclopropyl-4-(1H-indazol-1-yl)-1H-pyrazol-1-yl)cyclobutyl)methanol

To a solution of 1-(3-cyclopropyl-1H-pyrazol-4-yl)-1H-indazole (77 mg, 339.92 μmol) and cis-3-(hydroxymethyl)cyclobutyl 4-methylbenzenesulfonate (132.01 mg, 509.87 μmol) in DMSO (3 mL) was added Cs₂CO₃ (222.49 mg, 679.83 μmol). After the mixture was stirred at 100° C. for 3 h, it was diluted with EtOAc (10 mL), and washed with water and brine. The organic layer was concentrated and the residue was purified by silica gel chromatography (petroleum ether:EtOAc=1:9) to afford the title compound (82.2 mg, yield: 77%) as a white solid. MS (ESI) m/z=309.6 [M+H]⁺.

### Step 4. Synthesis of (trans-3-(3-cyclopropyl-4-(1H-indazol-1-yl)-1H-pyrazol-1-yl)cyclobutyl)methyl 4-methylbenzenesulfonate

To a solution of (trans-3-(3-cyclopropyl-4-(1H-indazol-1-yl)-1H-pyrazol-1-yl)cyclobutyl)methanol (77.78 mg, 249.69 μmol) in DCM (5 mL) were added TsCl (52.83 mg, 749.08 μmol), DMAP (30.51 mg, 249.69 μmol) and TEA (252.67 mg, 2.50 mmol). The mixture was stirred at 20 [symbol] for 3 h, before it was diluted with DCM (10 mL) and washed with H₂O and brine. The organic layer was concentrated, and the residue was purified by silica gel chromatography (petroleum ether:EtOAc=1:1) to afford the title compound (80.7 mg, yield: 69%) as a colorless oil. MS (ESI) m/z=463.8 [M+H]⁺.

### Step 5. Synthesis of tert-butyl (tert-butoxycarbonyl)((trans-3-(3-cyclopropyl-4-(H-indazol-1-yl)-1H-pyrazol-1-yl)cyclobutyl)methyl)carbamate

To a solution of (trans-3-(3-cyclopropyl-4-(1H-indazol-1-yl)-1H-pyrazol-1-yl)cyclobutyl)methyl 4-methylbenzenesulfonate (108.08 mg, 231.32 μmol) and tert-butyl N-tert-butoxycarbonylcarbamate (50.26 mg, 231.32 μmol) in DMSO (5 mL) was added Cs₂CO₃ (150.36 mg, 462.64 μmol). After the mixture was stirred at 100 [symbol] for 1 h, it was diluted with EtOAc (10 mL), and washed with water and brine. The organic layer was concentrated and the residue was purified by silica gel chromatography (petroleum ether:EtOAc=2:1) to afford the title compound (102.4 mg, yield: 86%) as a white solid. MS (ESI) m/z=508.7 [M+H]⁺.

### Step 6. Synthesis of (trans-3-(3-cyclopropyl-4-(1H-indazol-1-yl)-1H-pyrazol-1-yl)cyclobutyl)methanamine

To a stirred solution of tert-butyl (tert-butoxycarbonyl)((trans-3-(3-cyclopropyl-4-(1H-indazol-1-yl)-1H-pyrazol-1-yl)cyclobutyl)methyl)carbamate (101.01 mg, 197.00 μmol) in DCM (4 mL) was added HCl/dioxane (0.5 mL). The mixture was stirred at rt for 1 h, before it was concentrated. The residue was purified by silica gel chromatography to afford the title compound (44.3 mg, yield: 72%) as a white solid. MS (ESI) m/z=308.7 [M+H]⁺.

### Step 7. Synthesis of 5-(((trans-3-(3-cyclopropyl-4-(1H-indazol-1-yl)-1H-pyrazol-1-yl)cyclobutyl)methyl)amino)-2-(2.6-dioxopiperidin-3-yl)isoindoline-1,3-dione

To a solution of (trans-3-(3-cyclopropyl-4-(1H-indazol-1-yl)-1H-pyrazol-1-yl)cyclobutyl)methanamine (40.40 mg, 130.13 μmol) in DMSO (2 mL) were added 2-(2,6-dioxo-3-piperidyl)-5-fluoro-isoindoline-1,3-dione (35.94 mg, 130.13 μmol) and DIEA (50.37 mg, 390.39 μmol). The reaction mixture was stirred at 130 [symbol] under microwave irradiation for 1 h. The resulting mixture was purified by reverse phase chromatography (ACN in 0.05% TFA solution, 30% to 70%) to afford the title compound (12.1 mg, yield: 16%) as a yellow solid. MS (ESI) m/z=564.6 [M+H]⁺.

### Example 95. 5-(((Trans-3-(3-cyclopropyl-4-(1-isopropyl-1H-pyrrolo[2,3-b]pyridin-6-yl)-1H-pyrazol-1-yl)cyclobutyl)methyl)amino)-2-(2,6-dioxopiperidin-3-yl)isoindoline-1,3-dione (GS-737)

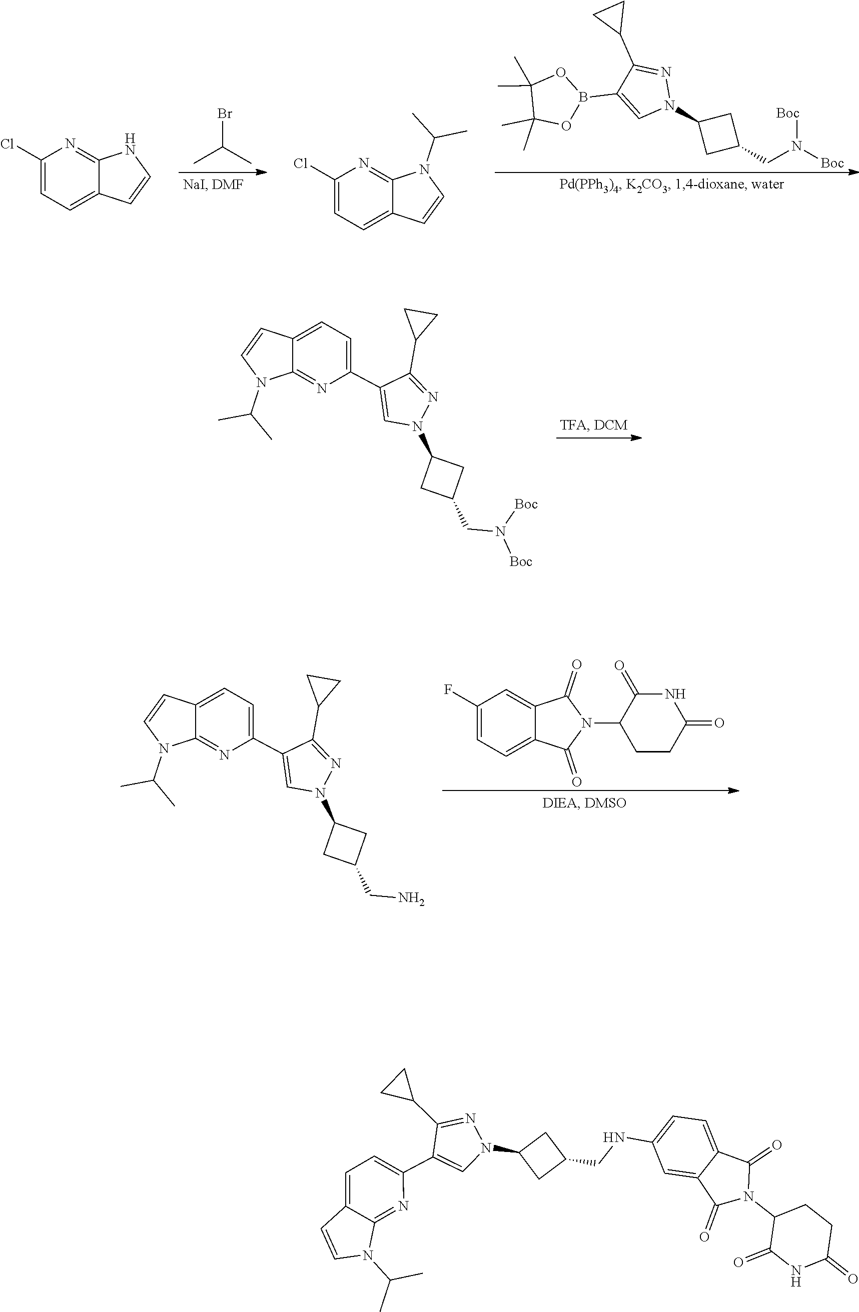

### Step 1. Synthesis of 6-chloro-1-isopropyl-1H-pyrrolo[2.3-b]pyridine

To a stirred solution of 6-chloro-1H-pyrrolo[2,3-b]pyridine (1.0 g, 6.55 mmol) in DMF (20 mL) was added NaH (531.31 mg, 21.92 mmol) under 0 [symbol] for 1 h. The mixture was stirred at 20 [symbol] for 1 h, before it was concentrated. The residue was purified by silica gel chromatography (petroleum ether EtOAc=10:1) to afford the title compound (970 mg, yield: 75%) as a colorless oil. MS (ESI) m/z=195.2 [M+H]⁺.

### Step 2. Synthesis of tert-butyl (tert-butoxycarbonyl)((trans-3-(3-cyclopropyl-4-(1-isopropyl-1H-pyrrolo[2,3-b]pyridin-6-yl)-1-pyrazol-1-yl)cyclobutyl)methyl)carbamate

To a solution of 6-chloro-1-isopropyl-1H-pyrrolo[2,3-b]pyridine (202.2 mg, 1.03 mmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (525.25 mg, 1.03 mmol) in 1,4-dioxane (10 mL) and H₂O (2 mL) were added Pd(PPh₃)₄ (115.3 mg, 99.57 μmol) and K₂CO₃ (280.8 mg, 2.06 mmol). The mixture was stirred at 100 [symbol] or 4 h under N₂, before it was concentrated. The residue was purified by silica gel chromatography (petroleum ether:EtOAc=1:1) to afford the title compound (300.6 mg, yield: 53%) as a colorless oil. MS (ESI) m/z=550.8 [M+H]⁺.

### Step 3. Synthesis of (trans-3-(3-cyclopropyl-4-(1-isopropyl-1H-pyrrolo[2,3-b]pyridin-6-yl)-1H-pyrazol-1-yl)cyclobutyl)methanamine

To a stirred solution of tert-butyl (tert-butoxycarbonyl)((trans-3-(3-cyclopropyl-4-(1-isopropyl-1H-pyrrolo[2,3-b]pyridin-6-yl)-1H-pyrazol-1-yl)cyclobutyl)methyl)carbamate (100.0 mg, 181.9 μmol) in DCM (5 mL) was added TFA (62.23 mg, 545.75 μmol). The mixture was stirred at 25° C. for 1 h, before it was concentrated. The residue was purified by silica gel chromatography to afford the title compound (53.4 mg, yield: 79%) as a white solid. MS (ESI) m/z=350.6 [M+H]⁺.

### Step 4. Synthesis of 5-(((trans-3-(3-cyclopropyl-4-(1-isopropyl-1H-pyrrolo[2,3-b]pyridin-6-yl)-1H-pyrazol-1-yl)cyclobutyl)methyl)amino)-2-(2,6-dioxopiperidin-3-yl)isoindoline-,3-dione

To a solution of (trans-3-(3-cyclopropyl-4-(1-isopropyl-1H-pyrrolo[2,3-b]pyridin-6-yl)-1H-pyrazol-1-yl)cyclobutyl)methanamine (53.4 mg, 143.07 μmol) in DMSO (2 mL) were added 2-(2,6-dioxo-3-piperidyl)-5-fluoro-isoindoline-1,3-dione (39.5 mg, 143.07 μmol) and DIEA (184.5 mg, 1.43 mmol). The reaction mixture was stirred at 130 [symbol] under microwave irradiation for 1 h. The resulting mixture was purified by reverse flash chromatography (ACN in water containing 0.05% TFA, 30% to 50%) to afford the title compound (15.3 mg, yield: 17%) as a yellow solid. MS (ESI) m/z=606.8 [M+H]⁺.

### Example 96. 5-(((Trans-3-(3-cyclopropyl-4-(1H-indol-1-yl)-1H-pyrazol-1-yl)cyclobutyl)methyl)amino)-2-(2,6-dioxopiperidin-3-yl)isoindoline-1,3-dione (GS-738)

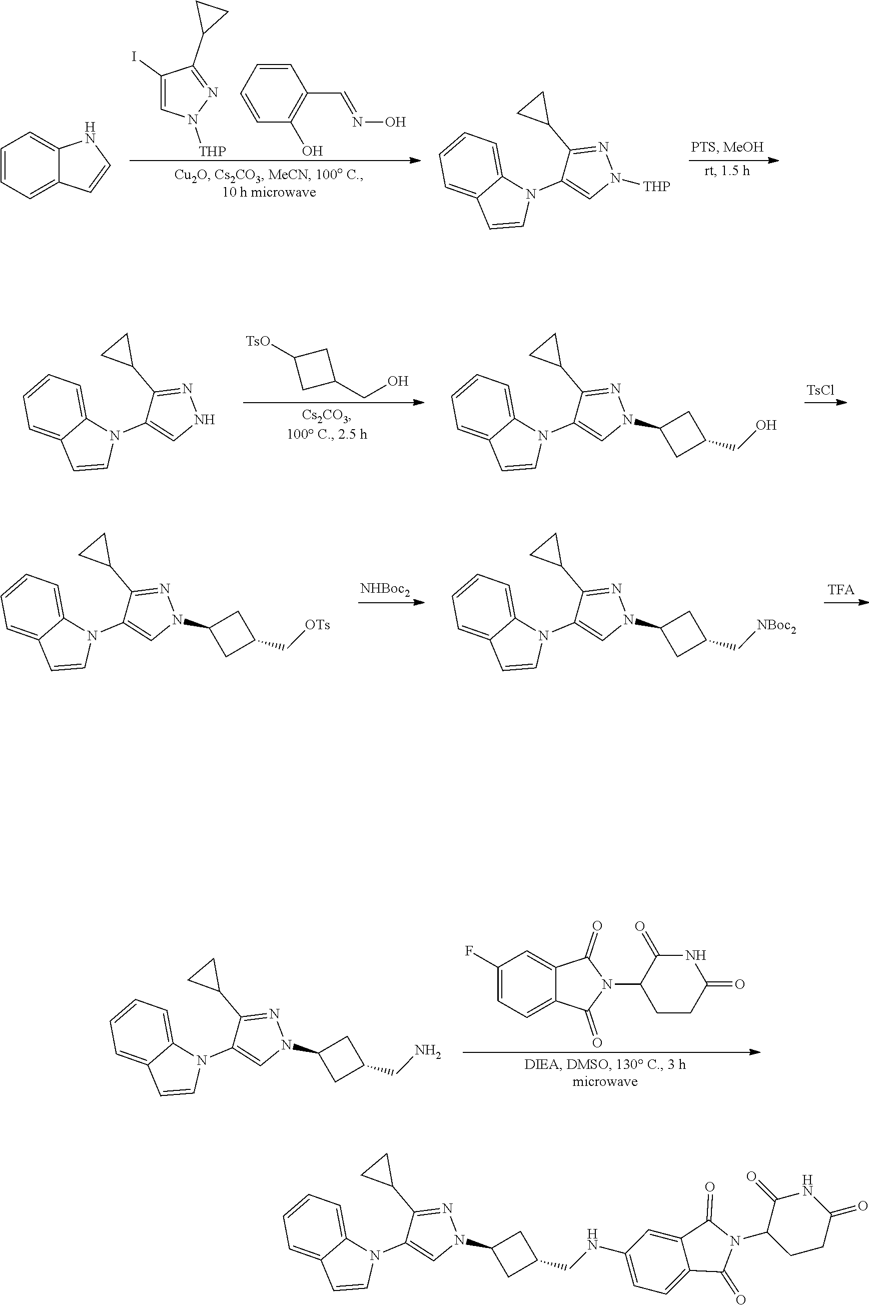

### Step 1. Synthesis of 1-(3-cyclopropyl-1-(tetrahydro-2H-pyran-2-yl)-1H-pyrazol-4-yl)-1H-indole

The title compound was synthesized following the similar procedure as described for step 1 of GS-736 (83.5 mg, yield: 11%) as oil. MS (ESI) m/z=308.4 [M+H]⁺.

### Step 2. Synthesis of 1-(3-cyclopropyl-1H-pyrazol-4-yl)-1H-indole

To a solution of 1-(3-cyclopropyl-1-(tetrahydro-2H-pyran-2-yl)-1H-pyrazol-4-yl)-1H-indole (230.2 mg, 748.89 μmol) in methanol (2.0 mL) was added PTS (644.80 mg, 3.74 mmol). The reaction mixture was stirred at rt for 1.5 h, before it was concentrated. The residue was extracted with EtOAc (20 mL×2). The combined organic layers were washed with sat. NaHCO₃ and brine. The organic layer was dried with Na₂SO₄, filtered and concentrated to afford the title compound (148.2 mg, yield: 89%) as a white solid. MS (ESI) m/z=224.6 [M+H]⁺.

### Step 3 to 7. Synthesis of 5-(((trans-3-(3-cyclopropyl-4-(1H-indol-1-yl)-1H-pyrazol-1-yl)cyclobutyl)methyl)amino)-2-(2.6-dioxopiperidin-3-yl)isoindoline-1,3-dione

GS-738 was synthesized following the procedures for steps 3 to 7 of GS-714 (TFA salt, 17.4 mg, yield: 4% over 5 steps) as a yellow solid. MS (ESI) m/z=563.5 [M+H]⁺.

### Example 97. 5-(((Trans-3-(3-Cyclopropyl-4-(phenylamino)-1H-pyrazol-1-yl)cyclobutyl)methyl)amino)-2-(2,6-dioxopiperidin-3-yl)isoindoline-1,3-dione (GS-739)

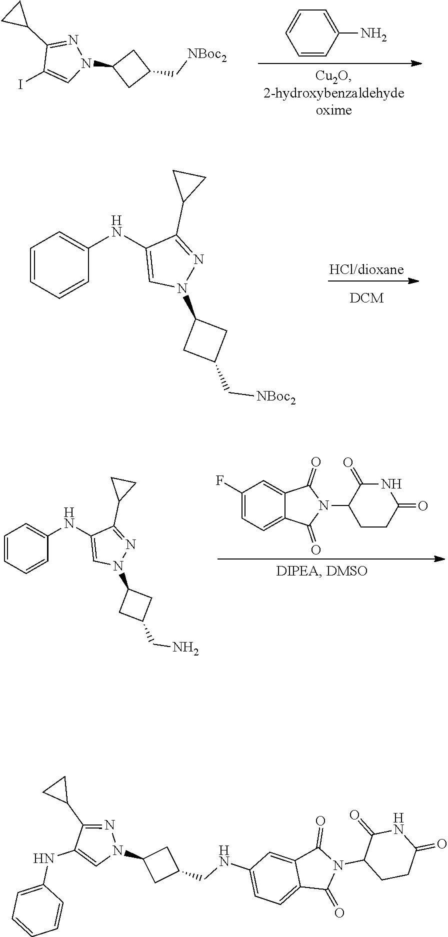

### Step 1. Synthesis of tert-butyl (tert-butoxycarbonyl)((trans-3-(3-cyclopropyl-4-(phenylamino)-1H-pyrazol-1-yl)cyclobutyl)methyl)carbamate

To a solution of aniline (269.98 mg, 2.90 mmol) and tert-butyl (tert-butoxycarbonyl)((trans-3-(3-cyclopropyl-4-iodo-1H-pyrazol-1-yl)cyclobutyl)methyl)carbamate (1.0 g, 1.93 mmol) in CH₃CN (10 mL) were added Cs₂CO₃ (942.21 mg, 2.90 mmol), Cu₂O (55.31 mg, 386.55 μmol) and 2-hydroxybenzaldehyde oxime (53.01 mg, 386.55 μmol) at rt under N₂. The mixture was warmed to 100 [symbol] for 12 h under microwave irradiation. After cooling to rt, the mixture was concentrated. The residue was purified by silica gel chromatography (petroleum ether:EtOAc=15:1 to 5:1) to afford the title compound (250 mg, yield: 27%). MS (ESI) m/z=483.9 [M+H]⁺.

### Step 2 to 3. Synthesis of 5-(((trans-3-(3-cyclopropyl-4-(phenylamino)-1H-pyrazol-1-yl)cyclobutyl)methyl)amino)-2-(2,6-dioxopiperidin-3-yl)isoindoline-1,3-dione

GS-739 was synthesized following the procedures for steps 6 to 7 of GS-736 (50 mg, yield: 18% over 2 steps) as a light-yellow solid. MS (ESI) m/z=539.7 [M+H]⁺.

### Example 98. 5-(((Trans-3-(3-cyclopropyl-5-(piperazin-1-yl)-1H-indazol-1-yl)cyclobutyl)methyl)amino)-2-(2,6-dioxopiperidin-3-yl)isoindoline-1,3-dione (GS-740)

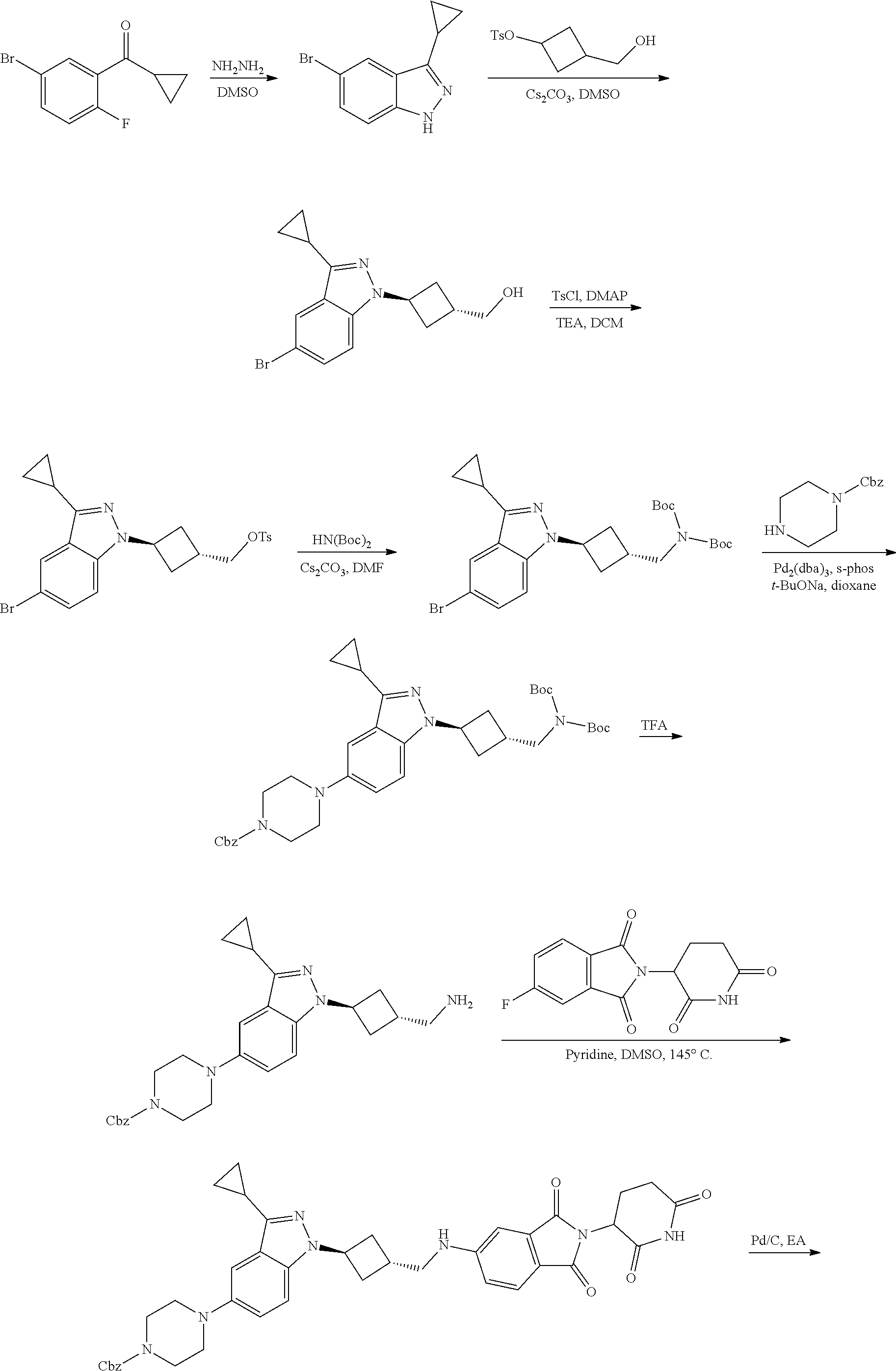

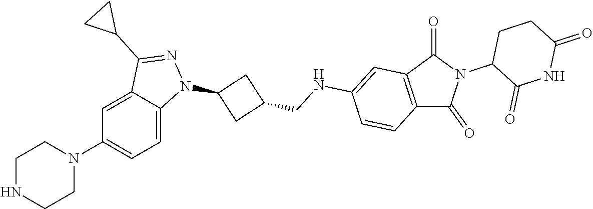 [SMILES: O=C1CCC(N2C(=O)c3ccc(NC[C@H]4C[C@H](n5nc(C6CC6)c6cc(N7CCNCC7)ccc65)C4)cc3C2=O)C(=O)N1]

### Step 1. Synthesis of 5-bromo-3-cyclopropyl-1H-indazole

To a solution of (5-bromo-2-fluoro-phenyl)-cyclopropyl-methanone (1 g, 4.11 mmol) in DMSO (3 mL) was added NH₂NH₂ (1.40 g, 41.14 mmol). The mixture was stirred at 130 [symbol] for 16 h, before it was filtered. The filtrate was concentrated to afford the title compound (730 mg, yield: 75%) as a white solid, which was used directly in the next step. MS (ESI) m/z=237.3 [M+H]⁺.

### Step 2. Synthesis of (trans-3-(5-bromo-3-cyclopropyl-1H-indazol-1-yl)cyclobutyl)methanol

To a solution of 5-bromo-3-cyclopropyl-1H-indazole (730 mg, 3.08 mmol) in DMF (5 mL) were added cis-3-(hydroxymethyl)cyclobutyl 4-methylbenzenesulfonate (789.18 mg, 3.08 mmol) and Cs₂CO₃ (2.00 g, 6.16 mmol). The mixture was stirred at 90 [symbol] for 16 h, before it was purified by reverse phase chromatography (water:MeOH=1:1) to afford the title compound (430 mg, yield: 43%) as a yellow oil. MS (ESI) m/z=321.2 [M+H]⁺.

### Step 3. Synthesis of (trans-3-(5-bromo-3-cyclopropyl-1H-indazol-1-yl)cyclobutyl)methyl 4-methylbenzenesulfonate

To a solution of (trans-3-(5-bromo-3-cyclopropyl-1H-indazol-1-yl)cyclobutyl)methanol (430 mg, 1.34 mmol) in DCM (20 mL) were added TEA (406.38 mg, 4.02 mmol), DMAP (16.35 mg, 133.87 μmol) and TsCl (381.52 mg, 2.01 mmol). The mixture was stirred at rt for 16 h, before it was concentrated. The residue was purified by silica gel chromatography (petroleum ether:EtOAc=5:1) to afford the title compound (440 mg, yield: 69%) as a yellow oil. MS (ESI) m/z=475.1 [M+H]⁺.

### Step 4. Synthesis of tert-butyl ((trans-3-(5-bromo-3-cyclopropyl-1H-indazol-1-yl)cyclobutyl)methyl)(tert-butoxycarbonyl)carbamate

To a solution of (trans-3-(5-bromo-3-cyclopropyl-1H-indazol-1-yl)cyclobutyl)methyl 4-methylbenzenesulfonate (300 mg, 631.05 μmol) in DMF (5 mL) were added tert-butyl N-tert-butoxycarbonylcarbamate (274.21 mg, 1.26 mmol) and Cs₂CO₃ (410.18 mg, 1.26 mmol). The mixture was stirred at 90 [symbol] for 1 h, before it was quenched with water (20 mL). The mixture was extracted with EtOAc (10 mL), dried, and concentrated. The residue was purified by reverse phase chromatography (0.1% TFA in water:MeOH=1:1) to afford the title compound (260 mg, yield: 79%) as a yellow solid. MS (ESI) m/z=520.2 [M+H]⁺.

### Step 5. Synthesis of benzyl 4-(1-(trans-3-((bis(tert-butoxycarbonyl)amino)methyl)cyclobutyl)-3-cyclopropyl-1H-indazol-5-yl)piperazine-1-carboxylate

To a solution of tert-butyl ((trans-3-(5-bromo-3-cyclopropyl-1H-indazol-1-yl)cyclobutyl)methyl)(tert-butoxycarbonyl)carbamate (260 mg, 499.56 μmol) in 1,4-dioxane (5 mL) were added benzyl piperazine-1-carboxylate (220.07 mg, 999.12 μmol), t-BuONa (95.92 mg, 999.12 μmol), Pd₂(dba)₃ (45.71 mg, 49.96 μmol) and S-phos (40.96 mg, 99.91 μmol). The mixture was stirred at 90 [symbol] for 1 h, before it was diluted with water (10 mL) and extracted with EtOAc (10 mL). The organic phase was concentrated to afford the title compound (260 mg, yield: 79%) as a yellow oil. MS (ESI) m/z=560.3 [M-100+H]⁺.

### Step 6. Synthesis of benzyl 4-(1-(trans-3-(aminomethyl)cyclobutyl)-3-cyclopropyl-1H-indazol-5-yl)piperazine-1-carboxylate

To a solution of benzyl 4-(1-(trans-3-((bis(tert-butoxycarbonyl)amino)methyl)cyclobutyl)-3-cyclopropyl-1H-indazol-5-yl)piperazine-1-carboxylate (260 mg, 394.05 μmol) in DCM (2 mL) was added TFA (1 mL). The mixture was stirred at rt for 30 min, before it was concentrated. The residue was purified by reverse phase chromatography (0.1% TFA in water:MeOH=1:1) to afford the title compound (150 mg, yield: 83%) as a yellow oil. MS (ESI) m/z=460.3 [M+H]⁺.

### Step 7. Synthesis of benzyl 4-(3-cyclopropyl-1-(trans-3-(((2-(2.6-dioxopiperidin-3-yl)-1,3-dioxoisoindolin-5-yl)amino)methyl)cyclobutyl)-1H-indazol-5-yl)piperazine-1-carboxylate

To a solution of benzyl 4-(1-(trans-3-(aminomethyl)cyclobutyl)-3-cyclopropyl-1H-indazol-5-yl)piperazine-1-carboxylate (150 mg, 326.38 μmol) in DMSO (5 mL) were added 2-(2,6-dioxo-3-piperidyl)-5-fluoro-isoindoline-1,3-dione (180.31 mg, 652.77 μmol) and pyridine (103.27 mg, 1.31 mmol). The mixture was stirred at 135 [symbol] for 4 h, before it was concentrated. The residue was purified by reverse phase chromatography (water:MeOH=1:1) to afford the title compound (56 mg, yield: 24%) as a yellow solid. MS (ESI) m/z=716.3 [M+H]⁺.

### Step 8. Synthesis of 5-(((trans-3-(3-cyclopropyl-5-(piperazin-1-yl)-1H-indazol-1-yl)cyclobutyl)methyl)amino)-2-(2,6-dioxopiperidin-3-yl)isoindoline-1,3-dione

To a solution of benzyl 4-(3-cyclopropyl-1-(trans-3-(((2-(2,6-dioxopiperidin-3-yl)-1,3-dioxoisoindolin-5-yl)amino)methyl)cyclobutyl)-1H-indazol-5-yl)piperazine-1-carboxylate (56 mg, 78.23 μmol) in EtOAc (10 mL) was added Pd/C (50 mg). The mixture was stirred at rt for 4 h under H₂, filtered and concentrated. The residue was purified by reverse phase chromatography (water:MeOH=1:1) to afford the title compound (3 mg, yield: 7%) as a yellow solid. MS (ESI) m/z=582.3 [M+H]⁺.

### Example 99. 5-((Trans-3-(5-benzyl-3-cyclopropyl-1H-pyrazol-1-yl)cyclobutyl)methylamino)-2-(2,6-dioxopiperidin-3-yl)isoindoline-1,3-dione (GS-741)

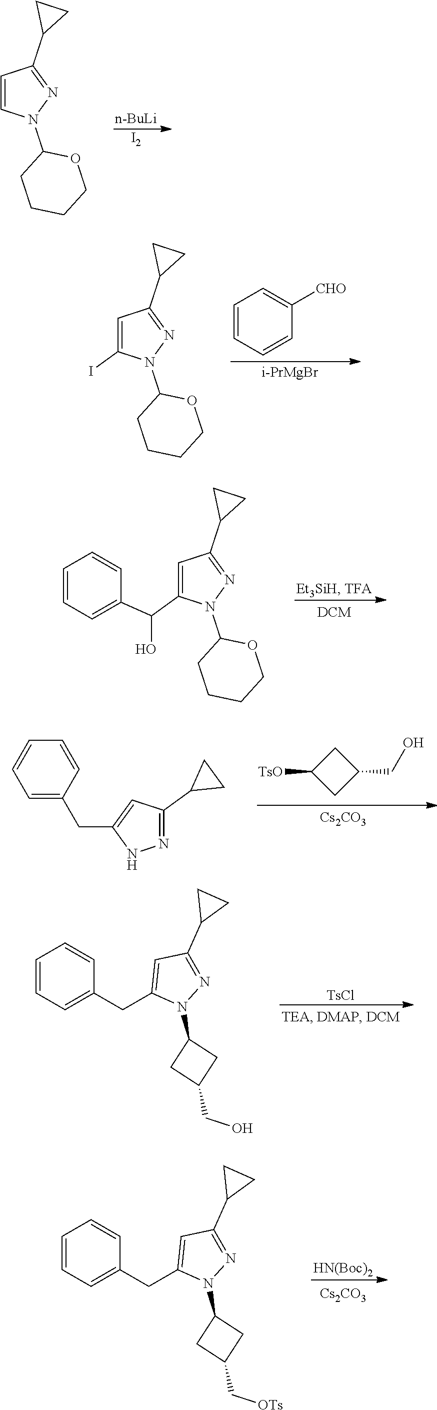

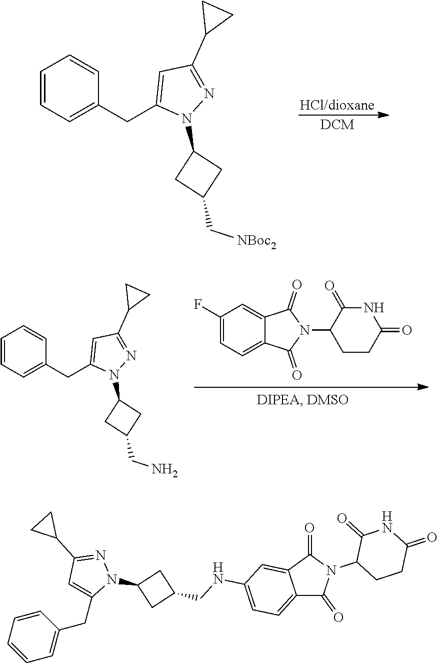

### Step 1. Synthesis of 3-cyclopropyl-5-iodo-1-(tetrahydro-2H-pyran-2-yl)-1H-pyrazole

To a solution of 3-cyclopropyl-1-tetrahydropyran-2-yl-pyrazole (2.0 g, 10.4 mmol) in THF (30 mL) was added n-BuLi (1.6 M in hexanes, 13.0 mL, 20.81 mmol) at −78 [symbol] . After the reaction was stirred for 30 min, I₂ (2.64 g, 10.40 mmol) in THF (20 mL) was added. After the mixture was stirred at −50 to −20 [symbol] for 2 h, it was quenched with water (10 mL), and extracted with ethyl acetate (50 mL×3). The combined organic layers were washed with brine, dried over Na₂SO₄ and concentrated. The residue was purified by silica gel chromatography to afford the title compound (2.6 g, yield: 79%) as a colorless solid. MS (ESI) m/z=319.6 [M+H]⁺.

### Step 2 to 6. Synthesis of tert-butyl ((trans-3-(5-benzyl-3-cyclopropyl-1H-pyrazol-1-yl)cyclobutyl)methyl)(tert-butoxycarbonyl)carbamate

The title compound was synthesized following the procedures for steps 1 to 3 of GS-735 and streps 3 to 5 of GS-714 (110.0 mg, yield: 9% over 5 steps). MS (ESI) m/z=482.5 [M+H]⁺.

### Step 7 to 8. Synthesis of 5-((trans-3-(5-benzyl-3-cyclopropyl-1H-pyrazol-1-yl)cyclobutyl)methylamino)-2-(2,6-dioxopiperidin-3-yl)isoindoline-1,3-dione

GS-741 was synthesized following the procedures for steps 6 to 7 of GS-736 (5.2 mg, yield: 4% over 2 steps). MS (ESI) m/z=538.7 [M+H]⁺.

### Example 100. 5-(((Trans-3-(3-cyclopropyl-6-(piperazin-1-yl)-1H-indazol-1-yl)cyclobutyl)methyl)amino)-2-(2,6-dioxopiperidin-3-yl)isoindoline-1,3-dione (GS-742)

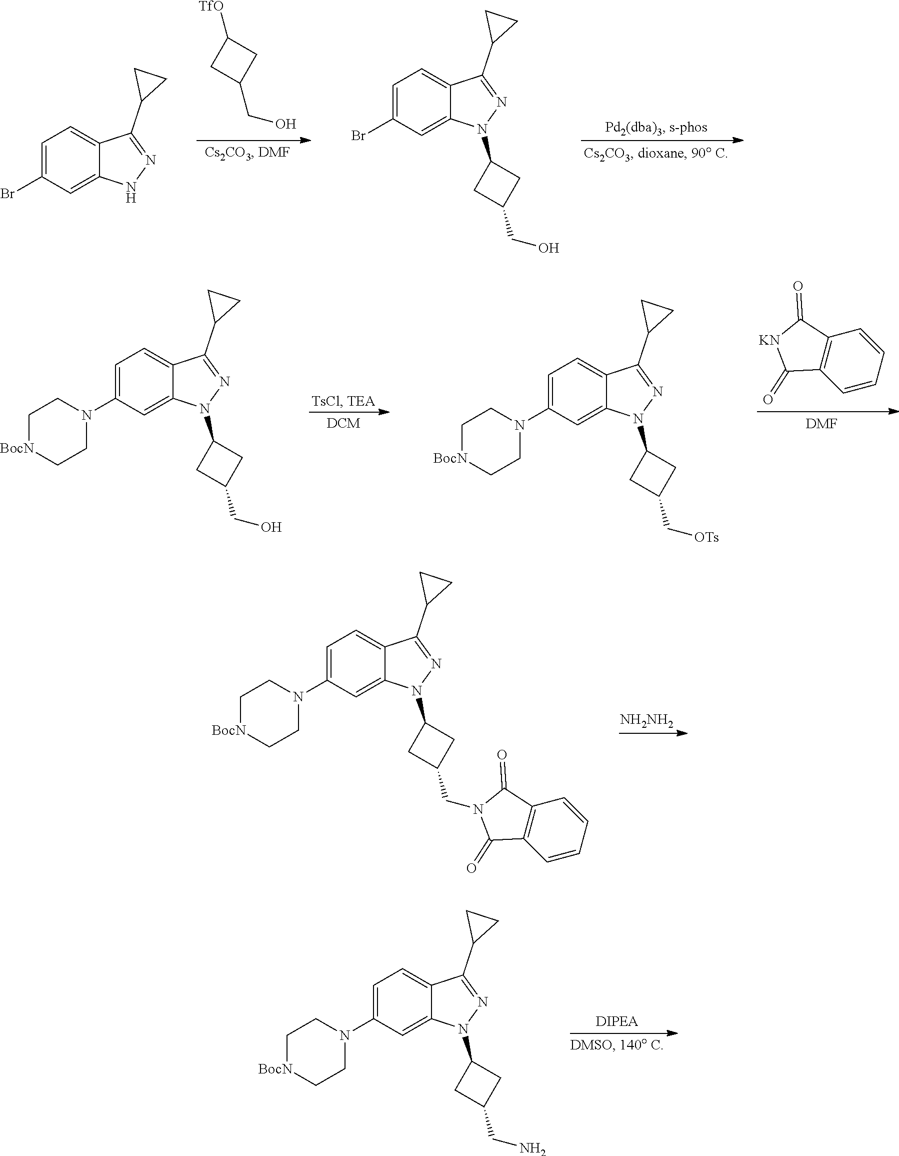

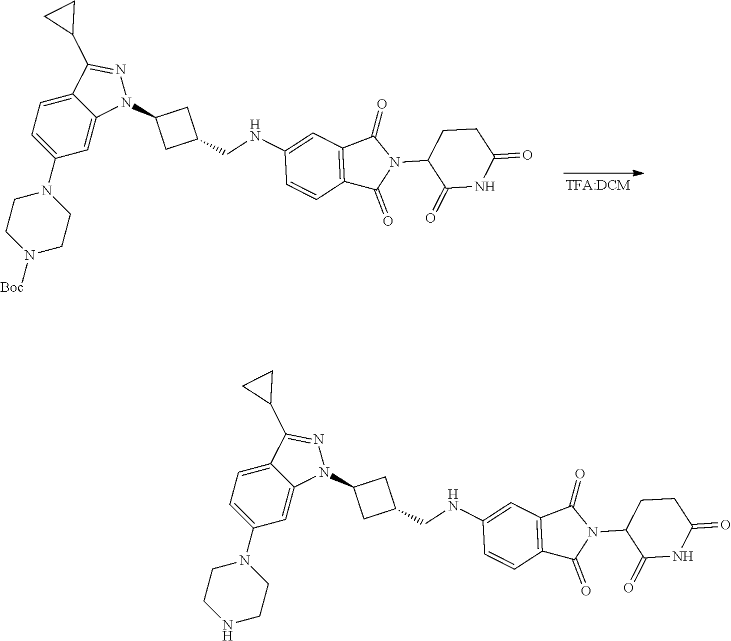 [SMILES: CC(C)(C)OC(=O)N1CCN(c2ccc3c(C4CC4)nn([C@H]4C[C@H](CNc5ccc6c(c5)C(=O)N(C5CCC(=O)NC5=O)C6=O)C4)c3c2)CC1 ; O=C1CCC(N2C(=O)c3ccc(NC[C@H]4C[C@H](n5nc(C6CC6)c6ccc(N7CCNCC7)cc65)C4)cc3C2=O)C(=O)N1]

### Step 1. Synthesis of (trans-3-(6-bromo-3-cyclopropyl-1H-indazol-1-yl)cyclobutyl)methanol

To a solution of 5-bromo-3-cyclopropyl-1H-indazole (500 mg, 2.11 mmol) in DMF (2 mL) were added Cs₂CO₃ (1.37 g, 4.22 mmol) and 3-(hydroxymethyl)cyclobutyl trifluoromethanesulfonate (810.80 mg, 3.16 mmol) under N₂. The mixture was purged with N₂ three times, before it was stirred at 90 [symbol] for 12 h. The mixture was partitioned between EtOAc (20 mL) and water (10 mL). The organic layer was dried and concentrated under reduced pressure. The resulting residue was purified by silica gel chromatography (petroleum ether:EtOAc=1:0 to 5:1) to afford the title compound (430 mg, yield: 60%). MS (ESI) m/z=321.3 [M+H]⁺.

### Step 2. Synthesis of tert-butyl 4-(3-cyclopropyl-1-(trans-3-(hydroxymethyl) cyclobutyl)-1H-indazol-6-yl)piperazine-1-carboxylate

To a solution of (trans-3-(6-bromo-3-cyclopropyl-1H-indazol-1-yl)cyclobutyl)methanol (50 mg, 155.66 μmol) in dioxane (2 mL) were added tert-butyl piperazine-1-carboxylate (57.98 mg, 311.32 μmol), Cs₂CO₃ (101.43 mg, 311.32 μmol), Pd₂(dba)₃ (14.25 mg, 15.57 μmol) and S-phos (12.78 mg, 31.13 μmol) subsequently under N₂. The mixture was purged with N₂ three times. The mixture was stirred at 90 [symbol] for 12 h, before it was filtered. The filtrate was concentrated under reduced pressure. The residue was purified by reverse phase chromatography (water:MeOH=1:0 to 1:2) to afford the title compound (24 mg, yield: 35%) as a yellow solid. MS (ESI) m/z=427.39 [M+H]⁺.

### Step 3. Synthesis of tert-butyl 4-(3-cyclopropyl-1-(trans-3-((tosyloxy)methyl) cyclobutyl)-1H-indazol-6-yl)piperazine-1-carboxylate

To a solution of tert-butyl 4-(3-cyclopropyl-1-(trans-3-(hydroxymethyl) cyclobutyl)-1H-indazol-6-yl)piperazine-1-carboxylate (24 mg, 56.27 μmol) in DCM (2 mL) were added DMAP (1.37 mg, 11.25 μmol), TEA (11.39 mg, 112.53 μmol) and TsCl (13.94 mg, 73.15 μmol) under N₂. The mixture was stirred at 25 [symbol] for 12 h, before it was diluted with DCM (20 mL) and washed with water (10 mL×2). The organic layer was concentrated under reduced pressure to afford the title compound (30 mg, yield: 92%), which was used directly in the next step. MS (ESI) m/z=581.34 [M+H]⁺.

### Step 4. Synthesis of tert-butyl 4-(3-cyclopropyl-1-(trans-3-((1,3-dioxoisoindolin-2-yl)methyl)cyclobutyl)-1H-indazol-6-yl)piperazine-1-carboxylate

To a solution of tert-butyl 4-(3-cyclopropyl-1-(trans-3-((tosyloxy)methyl) cyclobutyl)-1H-indazol-6-yl)piperazine-1-carboxylate (30 mg, 51.66 μmol) in DMF (3 mL) was added potassium 1,3-dioxoisoindolin-2-ide (14.35 mg, 77.49 μmol). The mixture was stirred at 90° C. for 1 h, before it was poured in EtOAc (20 mL) and washed with water (20 mL). The organic layer was concentrated under reduced pressure to afford the title compound (28 mg, yield: 98%), which was used into the next step without further purification. MS (ESI) m/z=556.35 [M+H]⁺.

### Step 5. Synthesis of tert-butyl 4-(1-(trans-3-(aminomethyl)cyclobutyl)-3-cyclopropyl-1H-indazol-6-yl)piperazine-1-carboxylate

To a solution of tert-butyl 4-(3-cyclopropyl-1-(trans-3-((1,3-dioxoisoindolin-2-yl)methyl)cyclobutyl)-1H-indazol-6-yl)piperazine-1-carboxylate (167 mg, 300.54 μmol) in ethanol (1.5 mL) was add hydrazinium hydroxide solution (50.39 μmol, 0.5 mL) under N₂. The mixture was stirred at 75 [symbol] for 1 h, before it was concentrated under reduced pressure. The resulting residue was purified by silica gel chromatography (DCM:MeOH=100:0 to 20:1) to afford the title compound (61 mg, yield: 48%). MS (ESI) m/z=426.40 [M+H]⁺.

### Step 6 to 7. Synthesis of 5-(((trans-3-(3-cyclopropyl-6-(piperazin-1-yl)-1H-indazol-1-yl)cyclobutyl)methyl)amino)-2-(2.6-dioxopiperidin-3-yl)isoindoline-1,3-dione

To a solution of tert-butyl 4-(1-(trans-3-(aminomethyl)cyclobutyl)-3-cyclopropyl-1H-indazol-6-yl)piperazine-1-carboxylate (61 mg, 143.34 μmol) and 2-(2,6-dioxo-3-piperidyl)-5-fluoro-isoindoline-1,3-dione (55.43 mg, 200.67 μmol) in DMSO (3 mL) was add DIPEA (74.10 mg, 573.35 μmol, 94.76 μL) under N₂. The mixture was stirred at 145 [symbol] for 2 h. After completion, the mixture was purified by reverse phase chromatography (water:CH₃CN=10:0 to 2:8) to afford the intermediate, which was dissolved in DCM (1 mL) and TFA (0.5 mL). The reaction mixture was stirred at 20 [symbol] for 1 h, before it was concentrated. The residue was purified by reverse phase chromatography (water:CH₃CN=10:0 to 4:6) to afford the title compound (2.5 mg, yield: 3%) as a yellow solid. MS (ESI) m/z=582.7 [M+H]⁺.

### Example 101. 5-(((Trans-3-(3-cyclopropyl-4-(7-fluoro-2-isopropyl-2H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-743)

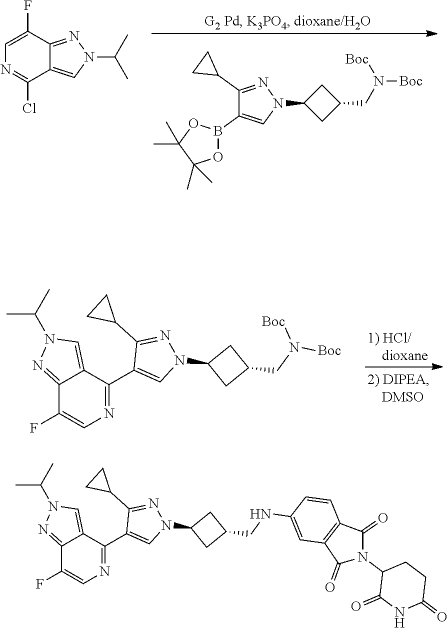 [SMILES: CC(C)n1cc2c(Cl)ncc(F)c2n1 ; CC(C)(C)OC(=O)N(C[C@H]1C[C@H](n2cc(B3OC(C)(C)C(C)(C)O3)c(C3CC3)n2)C1)C(=O)OC(C)(C)C ; CC(C)n1cc2c(-c3cn([C@H]4C[C@H](CN(C(=O)OC(C)(C)C)C(=O)OC(C)(C)C)C4)nc3C3CC3)ncc(F)c2n1 ; CC(C)n1cc2c(-c3cn([C@H]4C[C@H](CNc5ccc6c(c5)C(=O)N(C5CCC(=O)NC5=O)C6=O)C4)nc3C3CC3)ncc(F)c2n1]

GS-792 was synthesized following the procedures for steps 6 to 8 of GS-790 (5 mg, yield: 7% over 2 steps) as a yellow solid. MS (ESI) m/z=625.5 [M+H]⁺.

### Example 102. 5-((3-(Trans-3-(3-cyclopropyl-4-(quinoxalin-2-yl)-1H-pyrazol-1-yl)cyclobutyl)propyl)amino)-2-(2,6-dioxopiperidin-3-yl)-6-fluoroisoindoline-1,3-dione (GS-744)

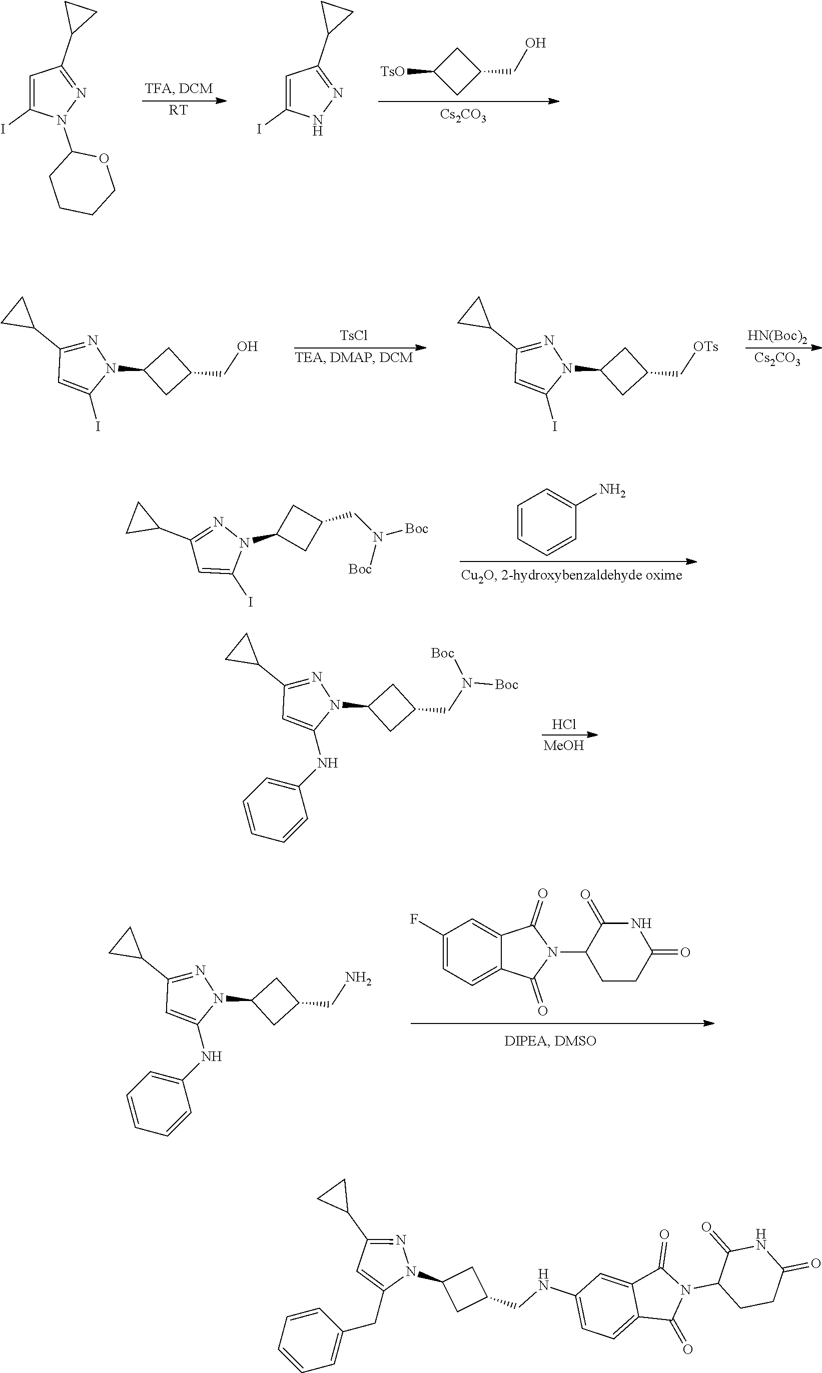

GS-744 was synthesized following the procedures for GS-739 (5.0 mg, yield: 1% over 7 steps) as a yellow solid. MS (ESI) m/z=539.6 [M+H]⁺.

### Example 103. 5-(((Trans-3-(4-(3-(azetidin-3-yl)pyridin-2-yl)-3-cyclopropyl-1H-pyrazol-1-yl)cyclobutyl)methyl)amino)-2-(2,6-dioxopiperidin-3-yl)isoindoline-1,3-dione (GS-745)

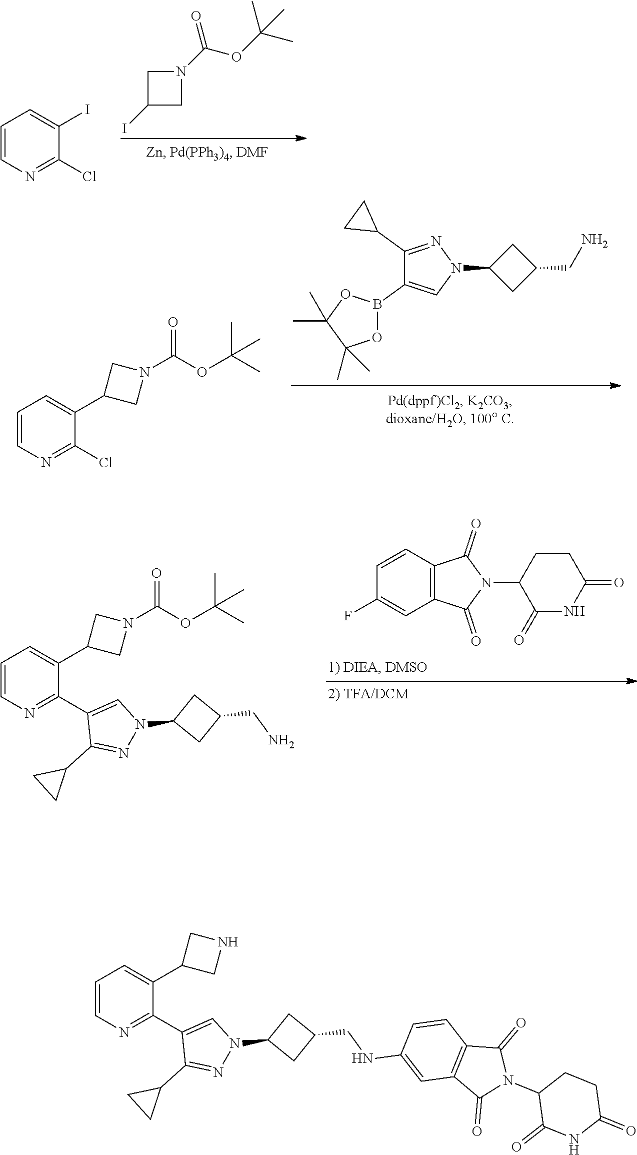

### Step 1. Synthesis of tert-butyl 3-(2-chloropyridin-3-yl)azetidine-1-carboxylate

To a suspension of zinc (2.73 g, 41.76 mmol) in DMF (10 mL) was added 1,2-dibromoethane (200 mg, 1.06 mmol) at rt under N₂. The mixture was stirred at 40 [symbol] for 1 h. TMSCl (200 mg, 4.18 mmol) was added. After the reaction was stirred at 40 [symbol] for 1 h, tert-butyl 3-iodoazetidine-1-carboxylate (5 g, 17.66 mmol) was added. The mixture was stirred at 40 [symbol] for another 1 h, before 2-Chloro-3-iodo-pyridine (1 g, 4.18 mmol) and Pd(PPh₃)₄ (50 mg, 4.18 mmol) were added. The mixture was stirred at 70 [symbol] overnight, before it was poured into sat. NH₄Cl solution (50 mL). The resulting mixture was extracted with EtOAc (50 mL×2). The combined organic layers were washed with brine (30 mL) and concentrated. The residue was purified by silica gel chromatography to afford the title compound (600 mg, yield: 42%) as a light yellow oil. MS (ESI) m/z=269.2 [M+H]⁺.

### Step 2. Synthesis of tert-butyl 3-(2-(1-(trans-3-(aminomethyl)cyclobutyl)-3-cyclopropyl-1H-pyrazol-4-yl)pyridin-3-yl)azetidine-1-carboxylate

A mixture of tert-butyl 3-(2-chloropyridin-3-yl)azetidine-1-carboxylate (50 mg, 141.40 μmol), (trans-3-(3-cyclopropyl-4-(4,4,5,5-tetramethyl-1,3,2-dioxaborolan-2-yl)-1H-pyrazol-1-yl)cyclobutyl)methanamine (100 mg, 176.53 μmol), Pd(dppf)Cl₂ (60 mg, 141.40 μmol) and K₂CO₃ (50 mg, 362.32 μmol) in dioxane (5 mL) and water (1 mL) was heated at 90 [symbol] overnight under N₂. The mixture was cooled to rt, before it was concentrated. The residue was purified by reverse phase chromatography to afford the title compound (30 mg, yield: 50%) as a light yellow oil. MS (ESI) m/z=424.4 [M+H]⁺.

### Step 3. Synthesis of 5-(((trans-3-(4-(3-(azetidin-3-v)pyridin-2-v)-3-cyclopropyl-1H-pyrazol-1-yl)cyclobutyl)methyl)amino)-2-(2,6-dioxopiperidin-3-yl)isoindoline-1,3-dione

A mixture of tert-butyl 3-(2-(1-(trans-3-(aminomethyl)cyclobutyl)-3-cyclopropyl-1H-pyrazol-4-yl)pyridin-3-yl)azetidine-1-carboxylate (30 mg, 70.12 μmol), 2-(2,6-dioxo-3-piperidyl)-5-fluoro-isoindoline-1,3-dione (40 mg, 144.81 μmol) and DIEA (50 mg, 387.60 μmol) in DMSO (1.5 mL) was heated at 130 [symbol] under N₂ for 6 h. The mixture was cooled to rt and purified by reverse phase chromatography to afford the intermediate as a yellow solid. The solid was dissolved in DCM (2 mL). TFA (1 mL) was added at rt. The mixture was stirred for 1 h, before it was concentrated. The residue was purified with prep-HPLC to afford the title compound (1.8 mg, yield: 4%) as a yellow solid. MS (ESI) m/z=580.6 [M+H]⁺.

### Example 104. 5-(((Trans-3-(3-cyclopropyl-4-(phenylamino)-1H-pyrazol-1-yl)cyclobutyl)methyl)amino)-2-(2,6-dioxopiperidin-3-yl)isoindoline-1,3-dione (GS-746)

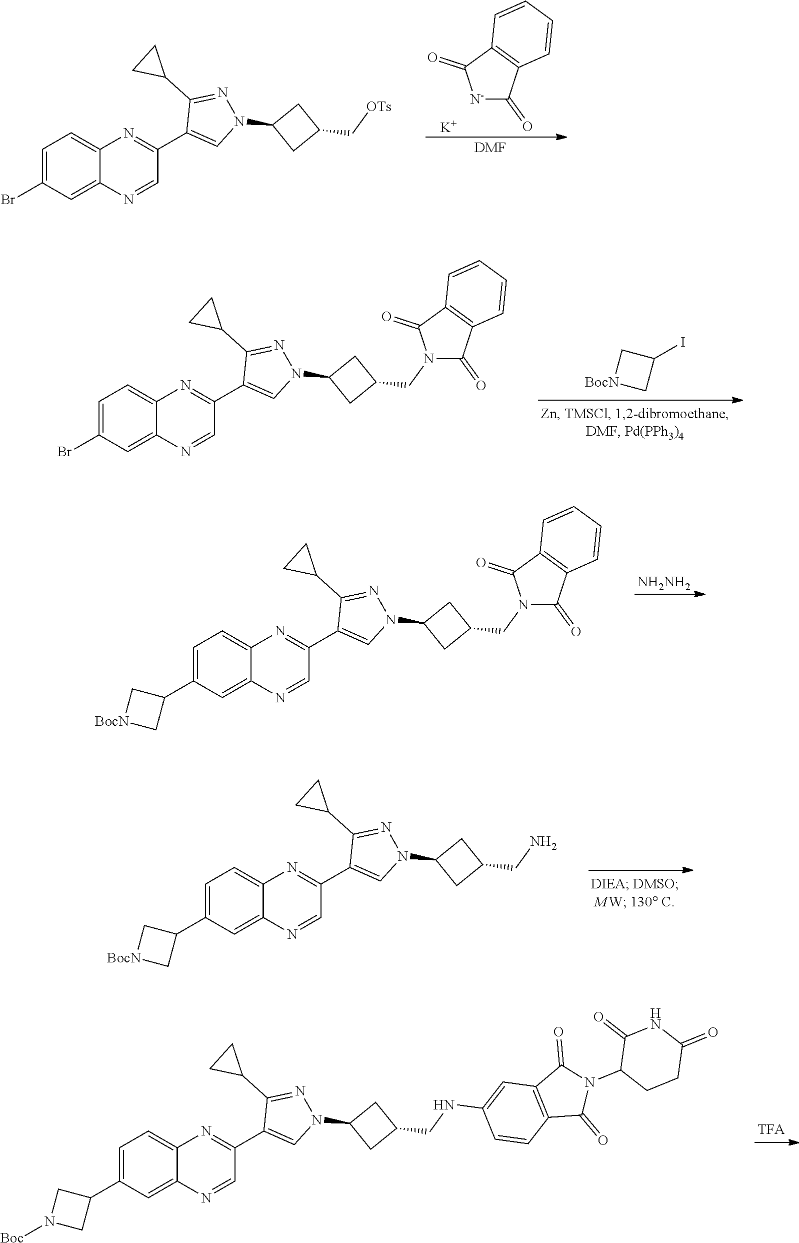

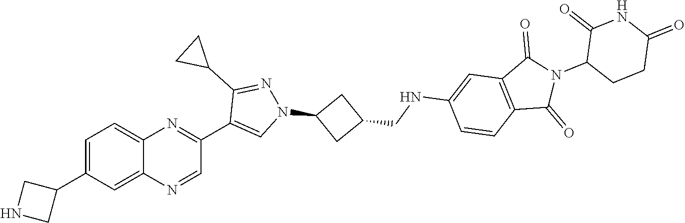 [SMILES: O=C1CCC(N2C(=O)c3ccc(NC[C@H]4C[C@H](n5cc(-c6cnc7cc(C8CNC8)ccc7n6)c(C6CC6)n5)C4)cc3C2=O)C(=O)N1]

### Step 1. Synthesis of 2-((trans-3-(4-(6-bromoquinoxalin-2-yl)-3-cyclopropyl-1H-pyrazol-1-yl)cyclobutyl)methyl)isoindoline-1,3-dione

To a solution of (trans-3-(4-(6-bromoquinoxalin-2-yl)-3-cyclopropyl-1H-pyrazol-1-yl)cyclobutyl)methyl 4-methylbenzenesulfonate (260 mg, 469.76 μmol) in DMSO (5 mL) was added potassium phthalimide (131.23 mg, 704.65 μmol). The mixture was stirred at 60° C. for 1 h, before it was purified by reverse phase chromatography (water:MeOH=1:10) to afford the title compound (220 mg, yield: 89%) as a yellow solid. MS (ESI) m/z=529.96 [M+H]⁺.

### Step 2. Synthesis of tert-butyl 3-(2-(3-cyclopropyl-1-(trans-3-((1,3-dioxoisoindolin-2-yl)methyl)cyclobutyl)-1H-pyrazol-4-yl)quinoxalin-6-yl)azetidine-1-carboxylate

To a suspension of Zn (43.05 mg, 662.38 μmol) in DMF (3 mL) was added TMSCl (1.44 mg, 13.25 μmol). After the mixture was stirred at 70° C. for 10 min, 1,2-dibromoethane (2.49 mg, 13.25 μmol) was added. After the mixture was stirred at 40 [symbol] for 1 h, tert-butyl 3-iodoazetidine-1-carboxylate (112.51 mg, 397.43 μmol) was added. After the mixture was stirred at 40 [symbol] for 1 h, 2-((trans-3-(4-(6-bromoquinoxalin-2-yl)-3-cyclopropyl-1H-pyrazol-1-yl)cyclobutyl)methyl)isoindoline-1,3-dione (70 mg, 132.48 μmol) and tetrakis(triphenylphosphine)palladium (7.65 mg, 6.62 μmol) were added. The mixture was stirred at 60 [symbol] for 16 h, before it was filtered and concentrated. The residue was purified by reverse phase chromatography (water:MeOH=1:1) to afford the title compound (21 mg, yield: 26%) as a yellow oil. MS (ESI) m/z=605.4 [M+H]⁺.

### Step 3 to 5. Synthesis of 5-(((trans-3-(3-cyclopropyl-4-(phenylamino)-1H-pyrazol-1-yl)cyclobutyl)methyl)amino)-2-(2,6-dioxopiperidin-3-yl)isoindoline-1,3-dione

GS-746 was synthesized following the procedures for steps 5 to 7 of GS-742 (0.2 mg, yield: 2% over 3 steps) as a yellow solid. MS (ESI) m/z=631.4 [M+H]⁺.

### Example 105. 5-(((Trans-3-(3-cyclopropyl-4-(1,4-dimethyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-747)

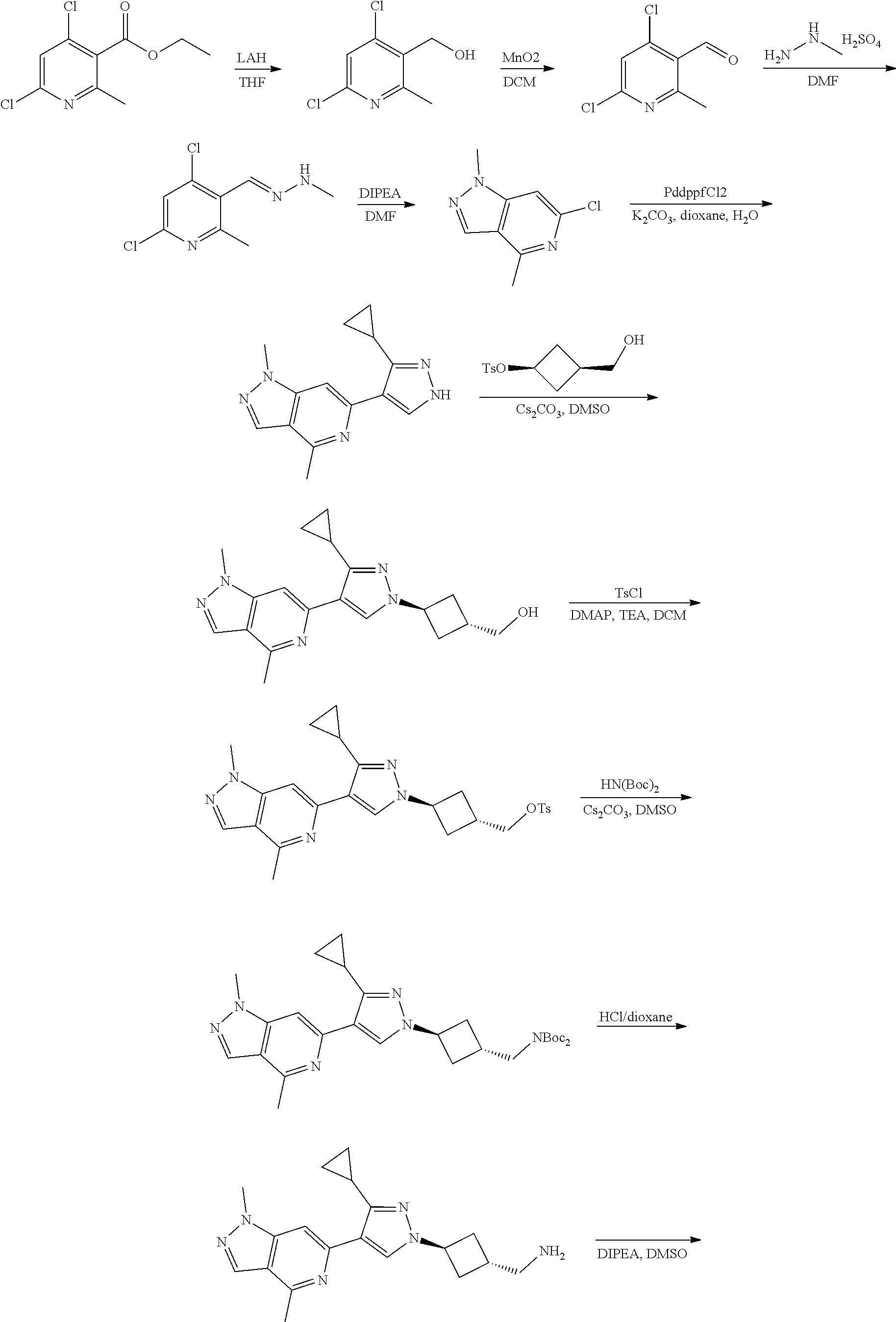

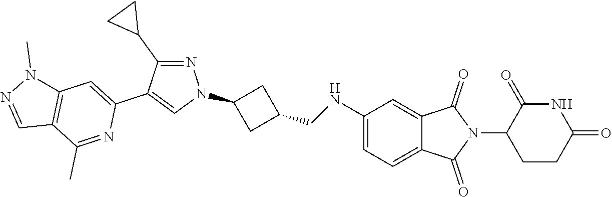 [SMILES: Cc1nc(-c2cn([C@H]3C[C@H](CNc4ccc5c(c4)C(=O)N(C4CCC(=O)NC4=O)C5=O)C3)nc2C2CC2)cc2c1cnn2C]

### Step 1. Synthesis of (4,6-dichloro-2-methyl-3-pyridyl)methanol

To a suspension of LAH (811.69 mg, 21.36 mmol) in THF (100 mL) was added solution ethyl 4,6-dichloro-2-methyl-pyridine-3-carboxylate (5.0 g, 21.36 mmol) in THF (20 mL) at 0 [symbol] slowly. The reaction mixture was stirred at 0 [symbol] for 0.5 h, before it was quenched with sat. Na₂SO₄ solution (0.9 mL) and water (0.9 mL). The resulting mixture was filtered, and the filtration was concentrated. The residue was purified by silica gel chromatography (petroleum ether:EtOAc=5:1) to afford the title compound (3.5 g, yield: 85%). MS (ESI) m/z=194.0 [M+H]⁺.

### Step 2. Synthesis of 4,6-dichloro-2-methyl-pyridine-3-carbaldehyde

To a solution of (4,6-dichloro-2-methyl-3-pyridyl)methanol (3.5 g, 18.23 mmol) in DCM (100 mL) was added MnO₂ (7.92 g, 91.13 mmol) at rt. The mixture was stirred at rt for 16 h, before it was filtered. The filtrate was concentrated to afford the title compound (3.0 g, yield: 87%), which was used directly in the next step. MS (ESI) m/z=191.9 [M+H]⁺.

### Step 3. Synthesis of 6-chloro-1,4-dimethyl-pyrazolo[4,3-c]pyridine

A solution of 4,6-dichloro-2-methyl-pyridine-3-carbaldehyde (3.0 g, 15.79 mmol) and methylhydrazine (H₂SO₄ salt, 3.41 g, 23.68 mmol) in DMF (80 mL) was warmed to 80 [symbol] for 0.5 h. DIPEA (8.16 g, 63.15 mmol, 10.44 mL) was added. The mixture was stirred at 120 [symbol] for 16 h. After cooling to rt, the mixture was purified by prep-HPLC to afford the title compound (2.0 g, yield: 70%). MS (ESI) m/z=182.1 [M+H]⁺.

### Step 4. Synthesis of 6-(3-cyclopropyl-1H-pyrazol-4-yl)-1,4-dimethyl-pyrazolo[4,3-c]pyridine

To a solution of 6-chloro-1,4-dimethyl-pyrazolo[4,3-c]pyridine (TFA salt, 2.0 g, 6.76 mmol) and tert-butyl 3-cyclopropyl-4-(4,4,5,5-tetramethyl-1,3,2-dioxaborolan-2-yl)pyrazole-1-carboxylate (2.71 g, 8.12 mmol) in dioxane (80 mL) and water (20 mL) were added Pd(dppf)Cl₂ (494.51 mg, 676.49 μmol) and K₂CO₃ (2.80 g, 20.29 mmol) at rt under N₂. The mixture was stirred at 100 [symbol] for 16 h, before it was concentrated. The residue was purified by silica gel chromatography (petroleum ether:EtOAc=10:1 to 1:1 to 1:2) to afford the title compound (1.2 g, yield: 70%). MS (ESI) m/z=254.4 [M+H]⁺.

### Step 5 to 9. Synthesis of (trans-3-(3-cyclopropyl-4-(1,4-dimethyl-1H-pyrazolo[4,3-c]pyridin-6-yl)-1-pyrazol-1-yl)cyclobutyl)methanol

GS-747 was synthesized following the procedures for steps 3 to 7 of GS-736 (7 mg, yield: 1% over 5 steps) as a light yellow solid. MS (ESI) m/z=593.6 [M+H]⁺.

### Example 106. 5-(((Trans-3-(3-cyclopropyl-4-(6-(pyrrolidin-1-yl)pyridin-2-yl)-1H-pyrazol-1-yl)cyclobutyl)methyl)amino)-2-(2,6-dioxopiperidin-3-yl)isoindoline-1,3-dione (GS-748)

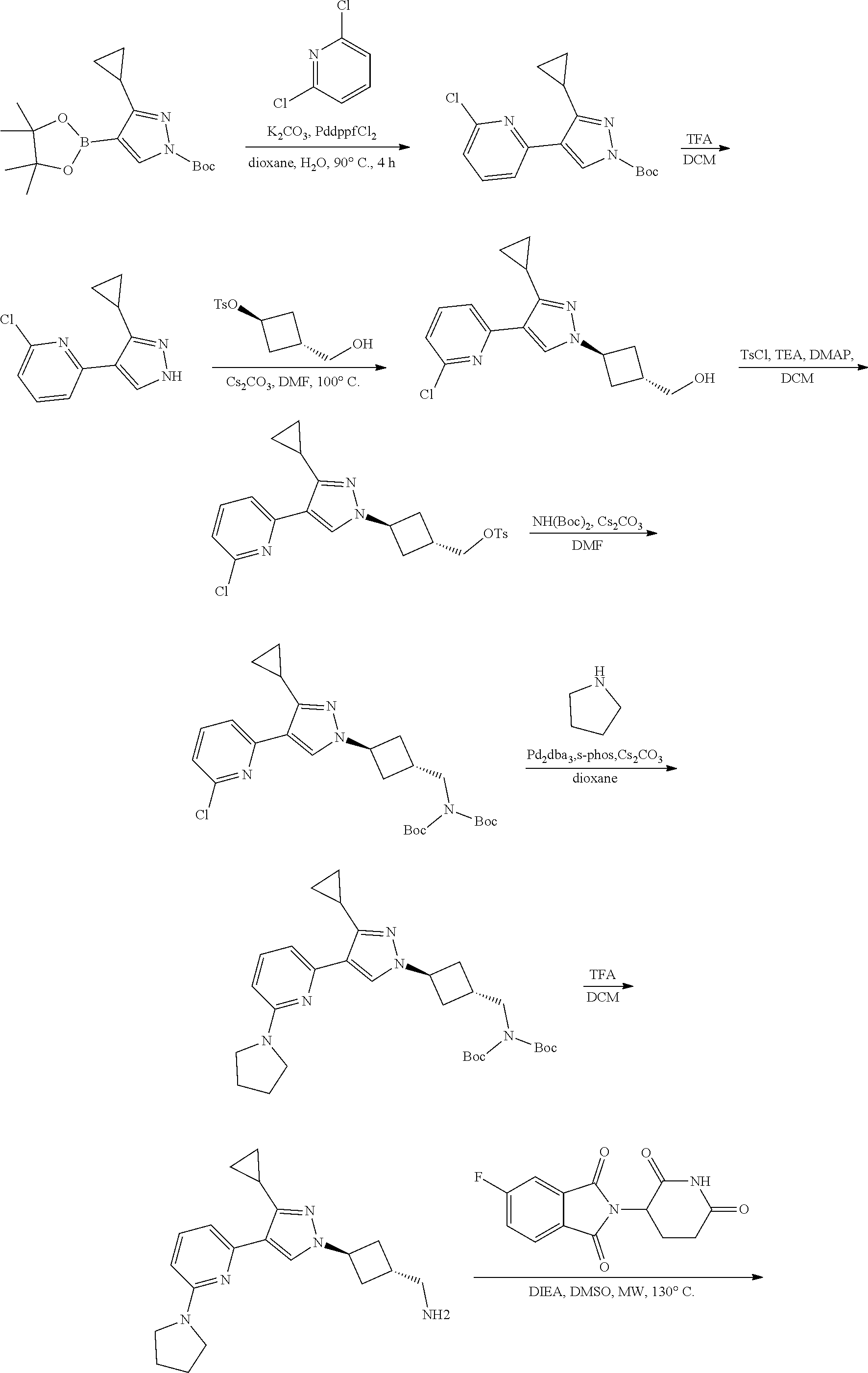

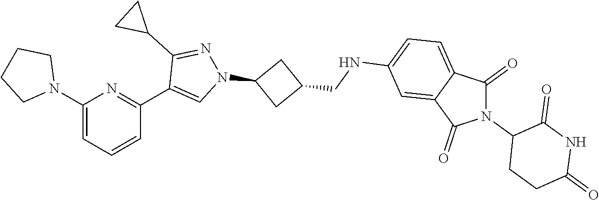 [SMILES: O=C1CCC(N2C(=O)c3ccc(NC[C@H]4C[C@H](n5cc(-c6cccc(N7CCCC7)n6)c(C6CC6)n5)C4)cc3C2=O)C(=O)N1]

### Step 1. Synthesis of tert-butyl 4-(6-chloropyridin-2-yl)-3-cyclopropyl-1H-pyrazole-1-carboxylate

To a solution of 2,6-dichloropyridine (3 g, 20.27 mmol) in dioxane (50 mL) and H₂O (10 mL) were added tert-butyl 3-cyclopropyl-4-(4,4,5,5-tetramethyl-1,3,2-dioxaborolan-2-yl)pyrazole-1-carboxylate (8.13 g, 24.33 mmol), K₂CO₃ (5.59 g, 40.54 mmol) and Pd(dppf)Cl₂ (2.96 g, 4.05 mmol). After the mixture was stirred at 80° C. for 2 h under Ar, it was concentrated and purified by reverse phase chromatography to afford the title compound (3.3 g, yield: 51%) as a yellow solid. MS (ESI) m/z=320.79 [M+H]⁺.

### Steps 2 to 5. Synthesis of tert-butyl (tert-butoxycarbonyl)((trans-3-(4-(6-chloropyridin-2-yl)-3-cyclopropyl-1H-pyrazol-1-yl)cyclobutyl)methyl)carbamate

The title compound was synthesized following the procedures for steps 2 to 5 of GS-717 (1.33 g, yield: 26% over 4 steps) as a light yellow solid. MS (ESI) m/z=504.04 [M+H]⁺.

### Step 6. Synthesis of tert-butyl (tert-butoxycarbonyl)((trans-3-(3-cyclopropyl-4-(6-(pyrrolidin-1-yl)pyridin-2-yl)-1H-pyrazol-1-yl)cyclobutyl)methyl)carbamate

To a solution of tert-butyl (tert-butoxycarbonyl)((trans-3-(4-(6-chloropyridin-2-yl)-3-cyclopropyl-1H-pyrazol-1-yl)cyclobutyl)methyl)carbamate (1.33 g, 2.64 mmol) and pyrrolidine (281.1 mg, 3.96 mmol) in dioxane (5 mL) were added Pd₂(dba)₃ (241 mg, 0.264 mmol), S-phos (216 mg, 0.528 mmol) and Cs₂CO₃ (2.58 g, 7.92 mmol). The reaction mixture was stirred at 100 [symbol] for 2 h under N₂, before it was diluted with water and extracted with EtOAc. The combined organic layers were washed with brine and dried over anhydrous Na₂SO₄. After filtration, the filtrate was concentrated under reduced pressure. The residue was purified by silica gel chromatography to afford the title compound (640 mg, yield: 45%) as a light yellow solid. MS (ESI) m/z=538.71 [M+H]⁺.

### Step 7. Synthesis of (trans-3-(3-cyclopropyl-4-(6-(pyrrolidin-1-yl)pyridin-2-yl)-1H-pyrazol-1-yl)cyclobutyl)methanamine

To a solution of tert-butyl (tert-butoxycarbonyl)((trans-3-(3-cyclopropyl-4-(6-(pyrrolidin-1-yl)pyridin-2-yl)-1H-pyrazol-1-yl)cyclobutyl)methyl)carbamate (640 mg, 1.19 mmol) in DCM (4 mL) was added TFA (2 mL). The reaction mixture was stirred at rt for 1 h, before it was concentrated. The residue was purified by reverse phase chromatography to afford the title compound (210 mg, yield: 52%) as a yellow solid. MS (ESI) m/z=338.47 [M+H]⁺.

### Step 8. Synthesis of 5-(((trans-3-(3-cyclopropyl-4-(6-(pyrrolidin-1-yl)pyridin-2-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(6-(pyrrolidin-1-yl)pyridin-2-yl)-1H-pyrazol-1-yl)cyclobutyl)methanamine (40 mg, 0.12 mmol) in DMSO (2 mL) were added 2-(2,6-dioxo-3-piperidyl)-5-fluoro-isoindoline-1,3-dione (36.4 mg, 0.13 mmol) and KF (69 mg, 1.2 mmol). The mixture was stirred at 130 [symbol] for 2 h under microwave irradiation, before it was purified by reverse phase chromatography to afford the title compound (15 mg, yield: 21%) as a yellow solid. MS (ESI) m/z=594.69 [M+H]⁺.

### Example 107. 5-((2-(6-(3-Cyclopropyl-4-(6-methylpyridin-2-yl)-1H-pyrazol-1-yl)spiro[3.3]heptan-2-yl)ethyl)amino)-2-(2,6-dioxopiperidin-3-yl)isoindoline-1,3-dione (GS-749)

### Step 1. Synthesis of tert-butyl 3-cyclopropyl-4-(6-methylpyridin-2-yl)-1H-pyrazole-1-carboxylate

To a solution of 2-bromo-6-methyl-pyridine (1 g, 5.81 mmol) in THF (50 mL) were added tert-butyl 3-cyclopropyl-4-(4,4,5,5-tetramethyl-1,3,2-dioxaborolan-2-yl)pyrazole-1-carboxylate (1.94 g, 5.81 mmol), Pd(PPh₃)₄ (335.71 mg, 290.66 μmol), K₂CO₃ (1.60 g, 11.63 mmol) and water (6 mL). The mixture was stirred at 60 [symbol] for 16 h, before it was concentrated to afford the title compound (1.2 g, yield: 30%) as a yellow oil. MS (ESI) m/z=300.2 [M+H]⁺.

### Step 2. Synthesis of 2-(3-cyclopropyl-1H-pyrazol-4-yl)-6-methylpyridine

To a solution of tert-butyl 3-cyclopropyl-4-(6-methylpyridin-2-yl)-1H-pyrazole-1-carboxylate (1.2 g, 1.72 mmol) in DCM (5 mL) was added TFA (5 mL). The mixture was stirred at rt for 1 h, before it was concentrated. The residue was purified by reverse phase chromatography (0.1% TFA in water:MeOH=1:1) to afford the title compound (190 mg, yield: 55%) as a yellow oil. MS (ESI) m/z=200.2 [M+H]⁺.

### Step 3. Synthesis of methyl 6-(3-cyclopropyl-4-(6-methylpyridin-2-yl)-1H-pyrazol-1-yl)spiro[3.3]heptane-2-carboxylate

To a solution of 2-(3-cyclopropyl-1H-pyrazol-4-yl)-6-methylpyridine (190 mg, 953.57 μmol) in DMF (5 mL) were added methyl 2-(p-tolylsulfonyloxy)spiro[3.3]heptane-6-carboxylate (463.99 mg, 1.43 mmol) and Cs₂CO₃ (619.82 mg, 1.91 mmol). The mixture was stirred at 90 [symbol] for 16 h. The residue was purified by reverse phase chromatography (0.1% TFA in water:MeOH=1:1) to afford the title compound (120 mg, yield: 36%) as a yellow oil. MS (ESI) m/z=352.3 [M+H]⁺.

### Step 4. Synthesis of (6-(3-cyclopropyl-4-(6-methylpyridin-2-yl)-1H-pyrazol-1-yl)spiro[3.3]heptan-2-yl)methanol

To a solution of methyl 6-(3-cyclopropyl-4-(6-methylpyridin-2-yl)-1H-pyrazol-1-yl)spiro[3.3]heptane-2-carboxylate (120 mg, 341.45 μmol) in THF (8 mL) was added LAH (23.17 mg, 682.90 μmol). The mixture was stirred at rt for 1 h, before 5 drops of water were added. The mixture was filtered, and the filtrated was concentrated. The residue was purified by reverse phase chromatography (water:MeOH=1:1) to afford the title compound (86 mg, yield: 78%) as a yellow oil. MS (ESI) m/z=324.2 [M+H]⁺.

### Step 5. Synthesis of (6-(3-cyclopropyl-4-(6-methylpyridin-2-yl)-1H-pyrazol-1-yl)spiro[3.3]heptan-2-yl)methyl 4-methylbenzenesulfonate

To a solution of (6-(3-cyclopropyl-4-(6-methylpyridin-2-yl)-1H-pyrazol-1-yl)spiro[3.3]heptan-2-yl)methanol (86 mg, 265.90 μmol) in DCM (10 mL) were added TsCl (75.78 mg, 398.85 μmol), TEA (80.72 mg, 797.70 μmol) and DMAP (3.25 mg, 26.59 μmol). The mixture was stirred at rt for 16 h, before it was concentrated. The residue was purified by silica gel chromatography (petroleum ether EtOAc=5:1) to afford the title compound (96 mg, yield: 76%) as a yellow oil. MS (ESI) m/z=478.2 [M+H]⁺.

### Step 6. Synthesis of 2-(6-(3-cyclopropyl-4-(6-methylpyridin-2-yl)-1H-pyrazol-1-yl)spiro[3.3]heptan-2-yl)acetonitrile

To a solution of (6-(3-cyclopropyl-4-(6-methylpyridin-2-yl)-1H-pyrazol-1-yl)spiro[3.3]heptan-2-yl)methyl 4-methylbenzenesulfonate (96 mg, 201.00 μmol) in MeCN (10 mL) were added TBAF (262.77 mg, 1.00 mmol) and trimethylsilyl cyanide (29.91 mg, 301.50 μmol). The mixture was stirred at 50 [symbol] for 16 h, before it was purified by reverse phase chromatography (water:MeOH=1:1) to afford the title compound (58 mg, yield: 87%) as a yellow oil. MS (ESI) m/z=333.2 [M+H]⁺.

### Step 7. Synthesis of 2-(6-(3-cyclopropyl-4-(6-methylpyridin-2-yl)-1H-pyrazol-1-yl)spiro[3.3]heptan-2-yl)ethan-1-amine

To a solution of 2-(6-(3-cyclopropyl-4-(6-methylpyridin-2-yl)-1H-pyrazol-1-yl)spiro[3.3]heptan-2-yl)acetonitrile (58 mg, 174.47 μmol) in THF (5 mL) were added ammonia (2.5 mL) and Pd/C (100 mg, 823.36 μmol). The mixture was stirred at rt for 1 h under H₂, before it was filtered, and concentrated to afford the title compound (52 mg, yield: 89%) as a colorless oil. MS (ESI) m/z=337.3 [M+H]⁺.

### Step 8. Synthesis of 5-((2-(6-(3-cyclopropyl-4-(6-methylpyridin-2-yl)-1H-pyrazol-1-yl)spiro[3.3]heptan-2-yl)ethyl)amino)-2-(2,6-dioxopiperidin-3-yl)isoindoline-1,3-dione

To a solution of 2-(6-(3-cyclopropyl-4-(6-methylpyridin-2-yl)-1H-pyrazol-1-yl)spiro[3.3]heptan-2-yl)ethan-1-amine (30 mg, 89.16 μmol) in DMSO (2 mL) were added 2-(2,6-dioxo-3-piperidyl)-5-fluoro-isoindoline-1,3-dione (24.63 mg, 89.16 μmol) and DIPEA (23.05 mg, 178.32 μmol, 29.47 μL). The mixture was stirred at 145 [symbol] for 2 h, before it was purified by reverse phase chromatography (water:MeOH=1:1) to afford the title compound (15 mg, yield: 28%) as a yellow solid. MS (ESI) m/z=593.3 [M+H]⁺.

### Example 108. 5-(((Trans-3-(3-cyclopropyl-4-(2-(3-hydroxycyclobutyl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-750)

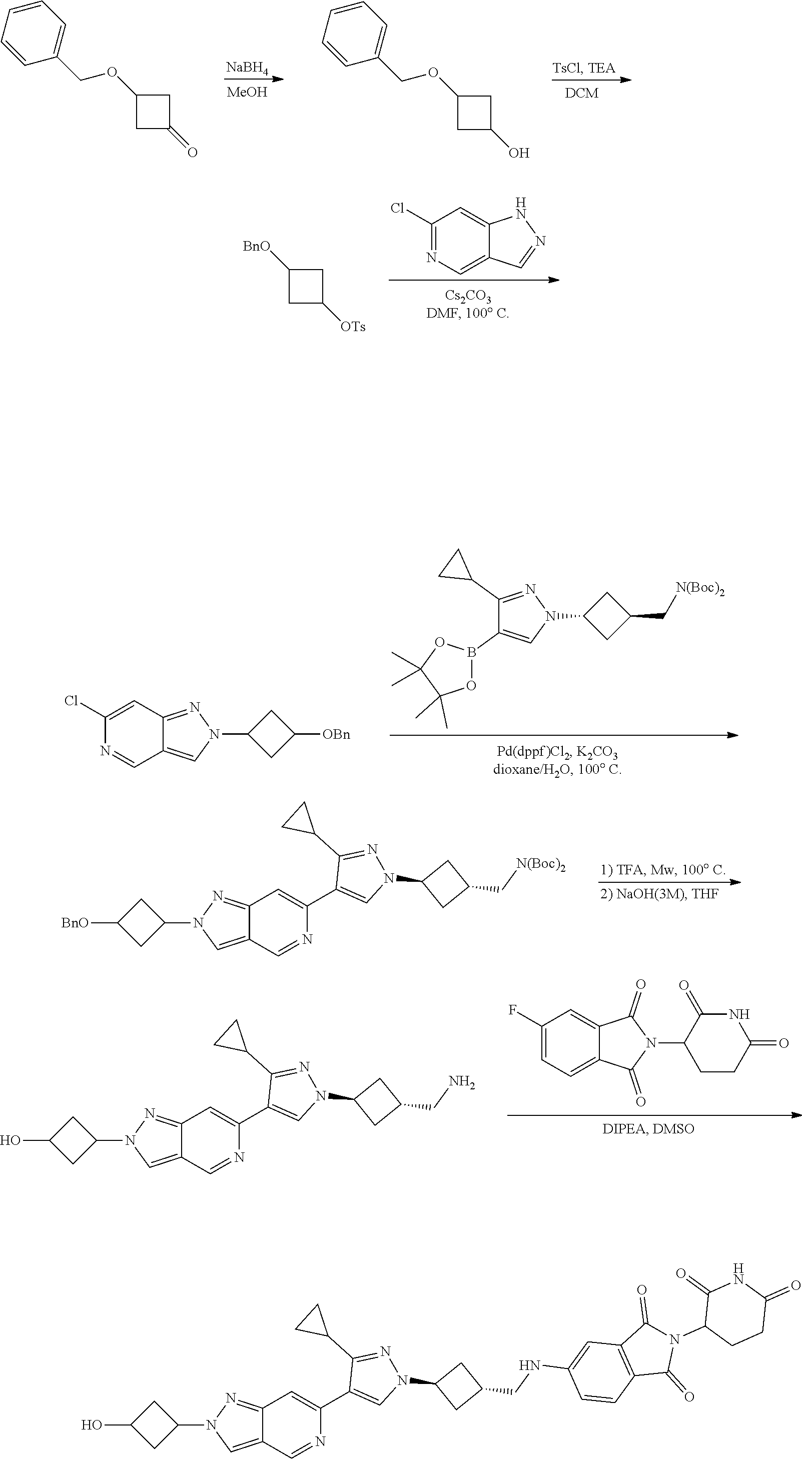

### Step 1. Synthesis of 3-(benzyloxy)cyclobutan-1-ol

To a solution of 3-benzyloxycyclobutanone (1 g, 5.68 mmol) in ethanol (10 mL) was added NaBH₄ (214.69 mg, 5.68 mmol). The mixture was stirred at 25 [symbol] for 0.5 h, before it was quenched with HCl (aq., 10%). The mixture was poured into water (15 mL) and extracted with DCM (20 mL×2). The combined organic layers were dried over anhydrous sodium sulfate and concentrated under reduced pressure to afford the title compound (1 g, yield: 94%), which was used directly in the next step.

### Step 2. Synthesis of 3-(benzyloxy)cyclobutyl 4-methylbenzenesulfonate

To a solution of 3-(benzyloxy)cyclobutan-1-ol (1.0 g, 5.61 mmol) in DCM (15 mL) were added TEA (1.14 g, 11.22 mmol), DMAP (68.55 mg, 0.56 mmol) and TsCl (593.63 mg, 8.42 mmol). The mixture was stirred at 25 [symbol] for 12 h, before it was washed with water (20 mL), aq. Na₂CO₃ solution (5%, 20 mL) and brine (20 mL) sequentially. The organic phase was dried over anhydrous sodium sulfate, filtered and concentrated under reduced pressure. The residue was purified by silica gel chromatography (petroleum ether:EtOAc=10:0 to 5:1) to afford the title compound (1.49 g, yield: 76%).

### Step 3. Synthesis of 2-(3-(benzyloxy)cyclobutyl)-6-chloro-2H-pyrazolo[4,3-c]pyridine

To a solution of 3-(benzyloxy)cyclobutyl 4-methylbenzenesulfonate (500 mg, 1.50 mmol) in DMF (4 mL) were added Cs₂CO₃ (735.12 mg, 2.26 mmol) and 6-chloro-1H-pyrazolo[4,3-c]pyridine (254.09 mg, 1.65 mmol). The mixture was stirred at 100 [symbol] for 1 h, before it was purified by silica gel chromatography (petroleum ether:EtOAc=1:0 to 3:1) to afford the title compound (300 mg, yield: 63%). MS (ESI) m/z=314.12 [M+H]⁺.

### Step 4. Synthesis of di-tert-butyl ((trans-3-(4-(2-(3-(benzyloxy)cyclobutyl)-2H-pyrazolo[4,3-c]pyridin-6-yl)-3-cyclopropyl-1H-pyrazol-1-yl)cyclobutyl)methyl)iminodicarbonate

To a solution of 2-(3-(benzyloxy)cyclobutyl)-6-chloro-2H-pyrazolo[4,3-c]pyridine (150 mg, 478.04 μmol) in dioxane (1.5 mL) and water (0.3 mL) were added tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (272.1 mg, 525.84 μmol), K₂CO₃ (132.14 mg, 956.08 μmol), and Pd(dppf)Cl₂ (34.98 mg, 47.80 μmol) under N₂. The mixture was then stirred at 100 [symbol] for 5 h, before it was filtered and concentrated under reduced pressure. The residue was purified by silica gel chromatography (petroleum ether:EtOAc=1:0 to 3:1) to afford the title compound (70 mg, yield: 22%). MS (ESI) m/z=669.41 [M+H]⁺.

### Step 5. Synthesis of 3-(6-(1-(trans-3-(aminomethyl)cyclobutyl)-3-cyclopropyl-1H-pyrazol-4-yl)-2H-pyrazolo[4,3-c]pyridin-2-yl)cyclobutan-1-ol

A solution of di-tert-butyl ((trans-3-(4-(2-(3-(benzyloxy)cyclobutyl)-2H-pyrazolo[4,3-c]pyridin-6-yl)-3-cyclopropyl-1H-pyrazol-1-yl)cyclobutyl)methyl)iminodicarbonate (60 mg, 89.71 μmol) in TFA (6.82 mg, 2 mL) was stirred at 100 [symbol] for 0.5 h under microwave irradiation. The mixture was concentrated under reduced pressure. The residue was dissolved in THF (2 mL). To the resulting solution was add NaOH (2.5 M, 295 L). The reaction mixture was stirred at 20 [symbol] for 0.5 h, before it was concentrated under reduced pressure. The residue was purified by reverse phase chromatography (CH₃CN:H₂O=1:0 to 3:1) to afford the title compound (33 mg, yield: 83%). MS (ESI) m/z=379.27 [M+H]⁺.

### Step 6. Synthesis of 5-(((trans-3-(3-cyclopropyl-4-(2-(3-hydroxycyclobutyl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

A solution of 3-(6-(1-(trans-3-(aminomethyl)cyclobutyl)-3-cyclopropyl-1H-pyrazol-4-yl)-2H-pyrazolo[4,3-c]pyridin-2-yl)cyclobutan-1-ol (10.00 mg, 26.42 μmol) and 2-(2,6-dioxo-3-piperidyl)-5-fluoro-isoindoline-1,3-dione (10.22 mg, 36.99 μmol) in DMSO (1.5 mL) was added DIPEA (13.66 mg, 105.69 μmol, 17.47 μL). The mixture was stirred at 145 [symbol] for 2 h under N₂, before it was purified by prep-HPLC to afford the title compound (3.3 mg, yield: 20%) as a yellow solid. MS (ESI) m/z=635.3 [M+H]⁺.

### Example 109. 5-(((Trans-3-(3-cyclopropyl-4-(1H-pyrrolo[2,3-b]pyridin-1-yl)-1H-pyrazol-1-yl)cyclobutyl)methyl)amino)-2-(2,6-dioxopiperidin-3-yl)isoindoline-1,3-dione (GS-751)

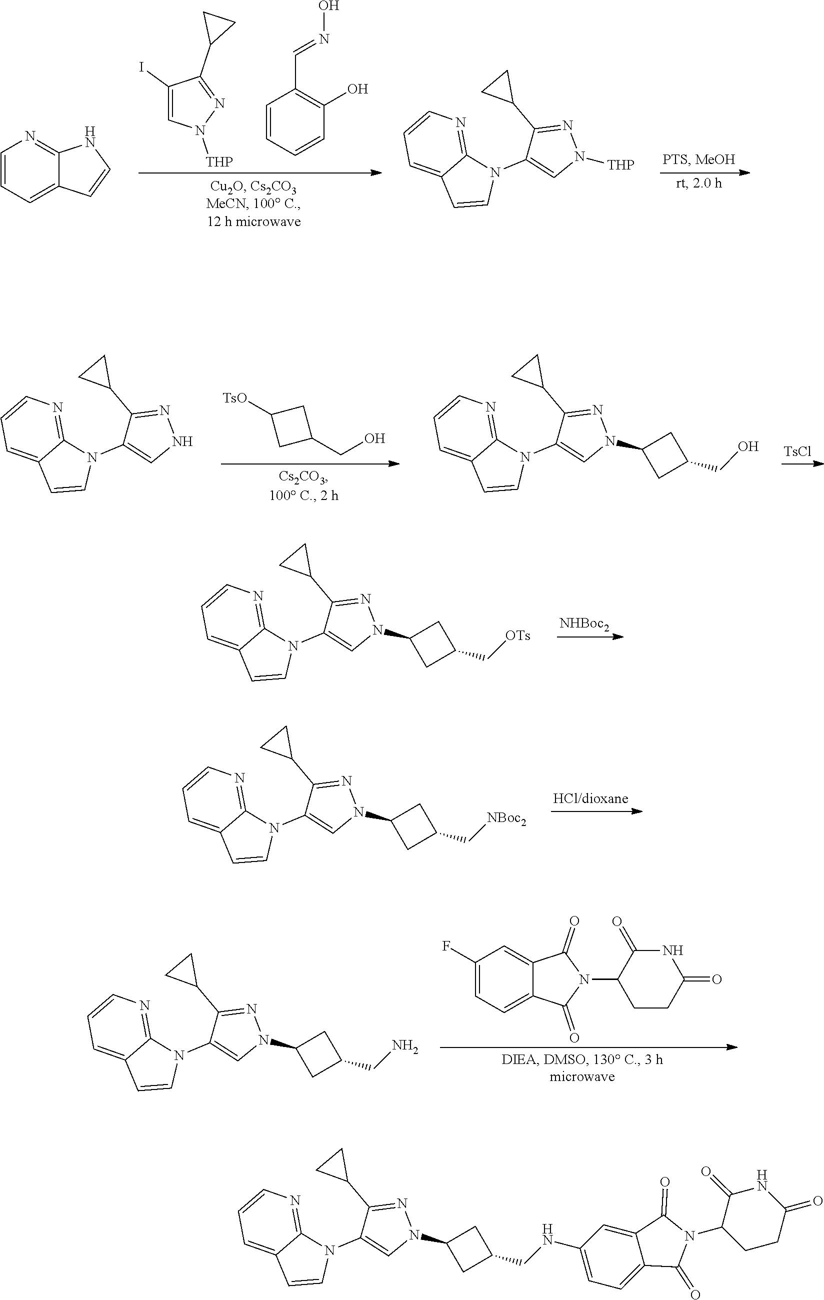

### Step 1. Synthesis of 1-(3-cyclopropyl-1-(tetrahydro-2H-pyran-2-yl)-H-pyrazol-4-yl)-1H-pyrrolo[2,3-b]pyridine

To a solution of 3-cyclopropyl-4-iodo-1-tetrahydropyran-2-yl-pyrazole (1.21 g, 3.81 mmol) and 11H-pyrrolo[2,3-b]pyridine (300 mg, 2.54 mmol) in MeCN (10 mL) were added 2-hydroxybenzaldehyde oxime (69.65 mg, 507.89 μmol), Cu₂O (18.17 mg, 126.97 μmol) and Cs₂CO₃ (1.65 g, 5.08 mmol). The reaction mixture was stirred at 100 [symbol] for 12 h under microwave irradiation, before it was filtered and concentrated. The residue was purified by silica gel chromatography (EtOAc:petroleum ether=1:4) to afford the title compound (164.5 mg, yield: 210%) as an oil. MS (ESI) m/z=309.3 [M+H]⁺.

### Step 2. Synthesis of 1-(3-cyclopropyl-1H-pyrazol-4-yl)-1H-pyrrolo[2,3-b]pyridine

To a solution of 1-(3-cyclopropyl-1-(tetrahydro-2H-pyran-2-yl)-1H-pyrazol-4-yl)-1H-pyrrolo[2,3-b]pyridine (164.5 mg, 533.44 μmol) in methanol (2.0 mL) was added PTS (459.77 mg, 2.67 mmol). The reaction mixture was stirred at rt for 2 h, before it was concentrated. The residue was diluted with water and extracted with EtOAc (20 mL×2). The combined organic layers were washed with sat. NaHCO₃ solution, dried over Na₂SO₄, filtered and concentrated to afford the title compound (114.6 mg, yield: 96%) as an oil. MS (ESI) m/z=225.5 [M+H]⁺.

### Step 3. Synthesis of (trans-3-(3-cyclopropyl-4-(1H-pyrrolo[2,3-b]pyridin-1-yl)-1H-pyrazol-1-yl)cyclobutyl)methanol

To a solution of 1-(3-cyclopropyl-1H-pyrazol-4-yl)-1H-pyrrolo[2,3-b]pyridine (114.6 mg, 511.01 μmol) and cis-3-(hydroxymethyl)cyclobutyl 4-methylbenzenesulfonate (157.18 mg, 613.21 μmol) in DMSO (2.0 mL) was added Cs₂CO₃ (499.49 mg, 1.53 mmol). The reaction mixture was stirred at 100 [symbol] for 2 h, before it was purified by reverse phase chromatography (MeOH:H₂O:TFA=50:50:0.5) to afford the title compound (83.5 mg, yield: 53%) as an oil. MS (ESI) m/z=309.6 [M+H]⁺.

### Step 4. Synthesis of (trans-3-(3-cyclopropyl-4-(1H-pyrrolo[2,3-b]pyridin-1-yl)-1H-pyrazol-1-yl)cyclobutyl)methyl 4-methylbenzenesulfonate

To a solution of (trans-3-(3-cyclopropyl-4-(1H-pyrrolo[2,3-b]pyridin-1-yl)-1H-pyrazol-1-yl)cyclobutyl)methanol (83.5 mg, 270.77 μmol) in DCM (2.0 mL) were added TsCl (103.24 mg, 541.55 μmol), DMAP (66.16 mg, 541.55 μmol) and TEA (137.00 mg, 1.35 mmol). The reaction mixture was stirred at rt for 2 h, before it was concentrated. The mixture was diluted with water and extracted with EtOAc (20 mL×2). The combined organic layers were washed with sat. NaHCO₃ solution and brine. The organic layer was dried with Na₂SO₄, filtered and concentrated. The residue was purified by silica gel chromatography (EtOAc:petroleum ether=15:85) to afford the title compound (68.7 mg, yield: 55%) as an oil. MS (ESI) m/z=463.7 [M+H]⁺.

### Step 5. Synthesis of tert-butyl (tert-butoxycarbonyl)((trans-3-(3-cyclopropyl-4-(1H-pyrrolo[2,3-b]pyridin-1-yl)-1H-pyrazol-1-yl)cyclobutyl)methyl)carbamate

To a solution of (trans-3-(3-cyclopropyl-4-(1H-pyrrolo[2,3-b]pyridin-1-yl)-1H-pyrazol-1-yl)cyclobutyl)methyl 4-methylbenzenesulfonate (68.7 mg, 148.52 μmol) in DMSO (2.0 mL) were added Cs₂CO₃ (96.78 mg, 297.04 μmol) and HNBoc₂ (35.49 mg, 163.37 μmol). The reaction mixture was stirred at 100 [symbol] for 1 h, before it was poured into ice/water. The mixture was extracted with EtOAc (20 mL×2). The combined organic layers were washed with brine, dried over Na₂SO₄, filtered and concentrated. The residue was purified by silica gel chromatography (EtOAc:petroleum ether=1:4) to afford the title compound (60.5 mg, yield: 80%) as an oil. MS (ESI) m/z=508.7 [M+H]⁺.

### Step 6. Synthesis of (trans-3-(3-cyclopropyl-4-(1H-pyrrolo[2,3-b]pyridin-1-yl)-1H-pyrazol-1-yl)cyclobutyl)methanamine

To a solution of tert-butyl (tert-butoxycarbonyl)((trans-3-(3-cyclopropyl-4-(1H-pyrrolo[2,3-b]pyridin-1-yl)-1H-pyrazol-1-yl)cyclobutyl)methyl)carbamate (60.5 mg, 119.18 μmol) in MeOH (0.5 mL) was added HCl (4 M in dioxane, 1.0 mL, 4 mmol). The reaction mixture was stirred at rt for 4 h, before it was concentrated to afford the title compound (HCl salt, 42.2 mg, yield: 78%) as a white solid. MS (ESI) m/z=308.4 [M+H]⁺.

### Step 7. Synthesis of 5-(((trans-3-(3-cyclopropyl-4-(1H-pyrrolo[2,3-b]pyridin-1-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(1H-pyrrolo[2,3-b]pyridin-1-yl)-1H-pyrazol-1-yl)cyclobutyl)methanamine (HCl salt, 42.2 mg, 93.11 μmol) and 2-(2,6-dioxo-3-piperidyl)-5-fluoro-isoindoline-1,3-dione (28.29 mg, 102.42 μmol) in DMSO (1.0 mL) was added DIPEA (120.33 mg, 931.08 μmol, 153.88 μL). The reaction mixture was stirred at 130 [symbol] for 3 h under microwave irradiation, before it was purified by reverse phase chromatography (MeCN:H₂O:TFA=50:50:0.5) to afford the title compound (TFA salt, 23.28 mg, yield: 28%) as a yellow solid. MS (ESI) m/z=564.7 [M+H]⁺.

### Example 110. 5-(((Trans-3-(4-(3-cyclopropoxy-5-fluoropyridin-2-yl)-3-cyclopropyl-1H-pyrazol-1-yl)cyclobutyl)methyl)amino)-2-(2,6-dioxopiperidin-3-yl)isoindoline-1,3-dione (GS-752)

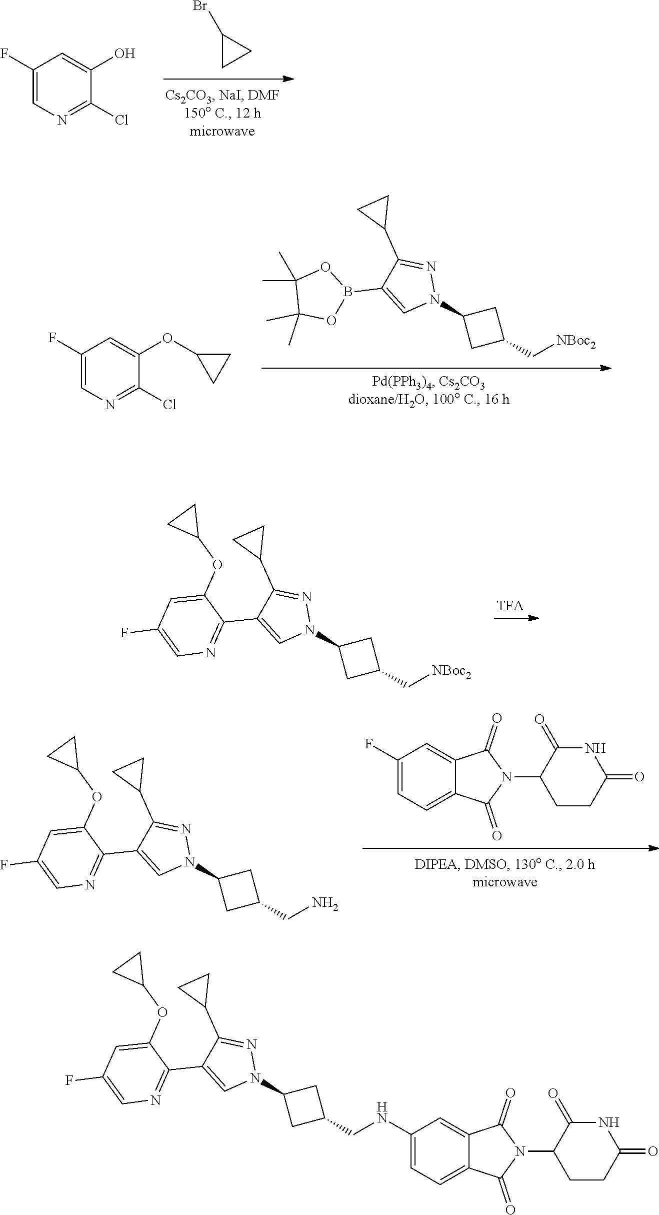

### Step 1. Synthesis of 2-chloro-3-cyclopropoxy-5-fluoropyridine

To a solution of 2-chloro-5-fluoro-pyridin-3-ol (500 mg, 3.39 mmol) and bromocyclopropane (4.10 g, 33.89 mmol, 2.72 mL) in DMF (5.0 mL) were added NaI (508.00 mg, 3.39 mmol) and Cs₂CO₃ (2.21 g, 6.78 mmol). The reaction mixture was stirred at 150 [symbol] for 12 h under microwave irradiation, before it was purified by silica gel chromatography (EtOAc:petroleum ether=3:17) to afford the title compound (307 mg, yield: 48%) as a red oil. MS (ESI) m/z=188.1 [M+H]⁺.

### Step 2. Synthesis of tert-butyl (tert-butoxycarbonyl)((trans-3-(4-(3-cyclopropoxy-5-fluoropyridin-2-yl)-3-cyclopropyl-1H-pyrazol-1-yl)cyclobutyl)methyl)carbamate

To a solution of 2-chloro-3-cyclopropoxy-5-fluoropyridine (100 mg, 533.05 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (413.75 mg, 799.58 μmol) in 1,4-dioxane (5.0 mL) and water (1.0 mL) were added Pd(PPh₃)₄ (61.60 mg, 53.31 μmol) and Cs₂CO₃ (521.04 mg, 1.60 mmol). The reaction mixture was stirred at 100 [symbol] for 16 h under Ar, before it was concentrated. The residue was purified by prep-TLC (EtOAc:petroleum ether=1:2) to afford the title compound (166 mg, yield: 57%) as an oil. MS (ESI) m/z=543.8 [M+H]⁺.

### Step 3. Synthesis of (trans-3-(4-(3-cyclopropoxy-S-fluoropyridin-2-yl)-3-cyclopropyl-1H-pyrazol-1-yl)cyclobutyl)methanamine

To a solution of tert-butyl (tert-butoxycarbonyl)((trans-3-(4-(3-cyclopropoxy-5-fluoropyridin-2-yl)-3-cyclopropyl-1H-pyrazol-1-yl)cyclobutyl)methyl)carbamate (166 mg, 305.91 μmol) in DCM (1.0 mL) was added TFA (34.88 mg, 305.91 μmol, 2.0 mL). The mixture was stirred at rt for 1 h, before it was concentrated to afford the title compound (TFA salt, 224.6 mg, yield: 92%) as a brown oil. MS (ESI) m/z=343.5 [M+H]⁺.

### Step 4. Synthesis of 5-(((trans-3-(4-(3-cyclopropoxy-5-fluoropyridin-2-yl)-3-cyclopropyl-1H-pyrazol-1-yl)cyclobutyl)methyl)amino)-2-(2,6-dioxopiperidin-3-yl)isoindoline-1,3-dione

To a solution of (trans-3-(4-(3-cyclopropoxy-5-fluoropyridin-2-yl)-3-cyclopropyl-1H-pyrazol-1-yl)cyclobutyl)methanamine (TFA salt, 224.6 mg, 426.58 μmol) and 2-(2,6-dioxo-3-piperidyl)-5-fluoro-isoindoline-1,3-dione (129.61 mg, 469.24 μmol) in DMSO (5.0 mL) was added DIPEA (551.32 mg, 4.27 mmol, 705.02 μL). The reaction mixture was stirred at 130 [symbol] for 2 h under microwave irradiation, before it was purified by reverse phase chromatography (MeCN:H₂O:TFA=50:50:0.5) to afford the title compound (TFA salt, 86.87 mg, yield: 22%) as a yellow solid. MS (ESI) m/z=599.7 [M+H]⁺.

### Example 111. 3-(5-(2-(Trans-3-(3-cyclopropyl-4-(5-fluoro-6-methylpyridin-2-yl)-1H-pyrazol-1-yl)cyclobutyl)ethyl)-1-oxoisoindolin-2-yl)piperidine-2,6-dione (GS-753)

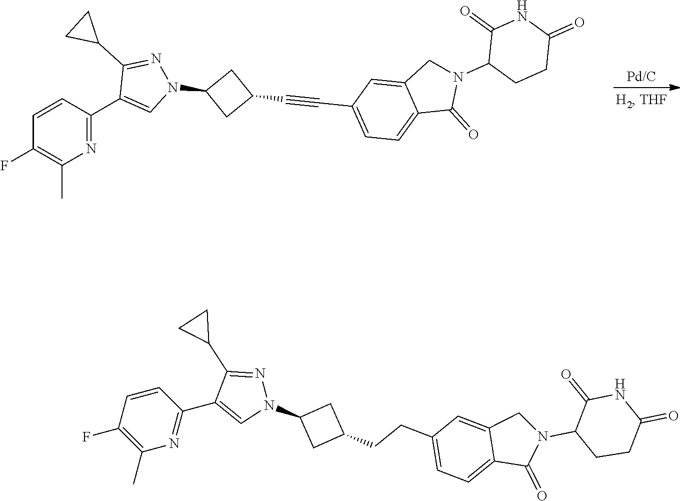 [SMILES: Cc1nc(-c2cn([C@H]3C[C@H](C#Cc4ccc5c(c4)CN(C4CCC(=O)NC4=O)C5=O)C3)nc2C2CC2)ccc1F ; Cc1nc(-c2cn([C@H]3C[C@H](CCc4ccc5c(c4)CN(C4CCC(=O)NC4=O)C5=O)C3)nc2C2CC2)ccc1F]

To a solution of 3-(5-((trans-3-(3-cyclopropyl-4-(5-fluoro-6-methylpyridin-2-yl)-1H-pyrazol-1-yl)cyclobutyl)ethynyl)-1-oxoisoindolin-2-yl)piperidine-2,6-dione (100 mg, 186.02 μmol) in THF (5 mL) and MeOH (5 mL) was added Pd/C (22.59 mg, 186.02 μmol). The mixture was stirred at rt under H₂ for 2 h, before it was filtered. The filtration was concentrated. The residue was purified by prep-TLC (DCM:MeOH=20:1) to afford the title compound (88 mg, yield: 87%) as a white solid. MS (ESI) m/z=542.8 [M+H]⁺.

### Example 112. 3-(5-((Trans-3-(3-cyclopropyl-4-(5-fluoro-6-methylpyridin-2-yl)-1H-pyrazol-1-yl)cyclobutyl)ethynyl)-1-oxoisoindolin-2-yl)piperidine-2,6-dione (GS-754)

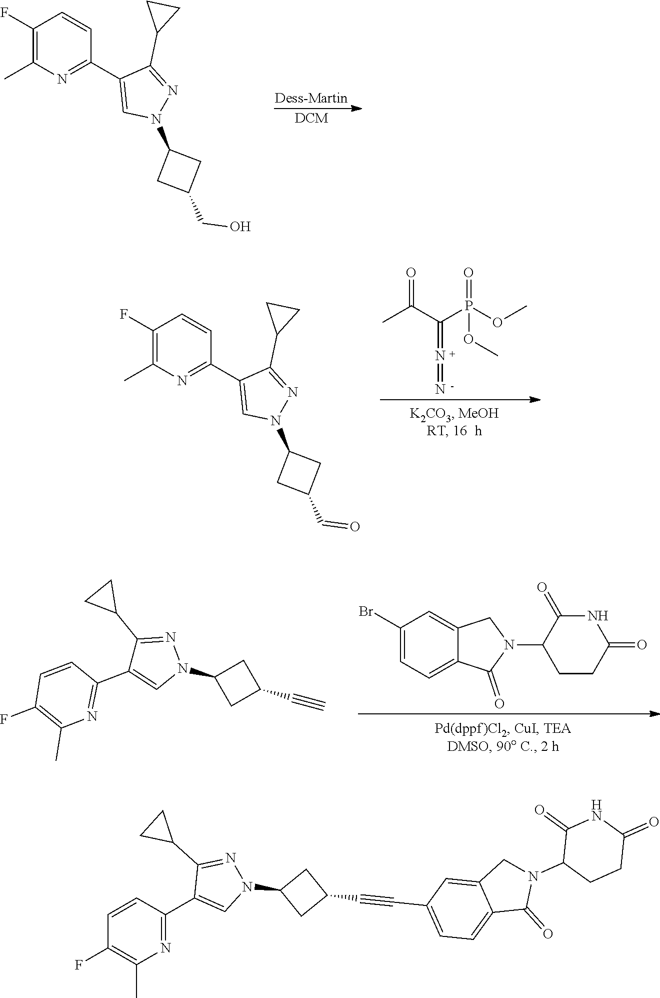

### Step 1 to 2. Synthesis of 6-(3-cyclopropyl-1-(trans-3-ethynylcyclobutyl)-1H-pyrazol-4-yl)-3-fluoro-2-methylpyridine

To a solution of (trans-3-(3-cyclopropyl-4-(5-fluoro-6-methylpyridin-2-yl)-1H-pyrazol-1-yl)cyclobutyl)methanol (0.9 g, 2.99 mmol) in DCM (40 mL) was added Dess-Martin periodinane (2.53 g, 5.97 mmol, 1.85 mL) at rt. The mixture was stirred at rt for 2 h, before it was quenched with water. The organic phase was dried over Na₂SO₄ and concentrated. To the residue were added MeOH (40 mL). K₂CO₃ (2.06 g, 14.93 mmol) and 1-diazo-1-dimethoxyphosphoryl-propan-2-one (1.15 g, 5.97 mmol) subsequentially. The reaction mixture was stirred at rt for 16 h, before it was concentrated. The residue was purified by silica gel chromatography (petroleum ether:EtOAc=10:1 to 5:1) to afford the title compound (420 mg, yield: 48%). MS (ESI) m/z=296.6 [M+H]⁺.

### Step 3. Synthesis of 3-(5-((trans-3-(3-cyclopropyl-4-(5-fluoro-6-methylpyridin-2-yl)-1H-pyrazol-1-yl)cyclobutyl)ethynyl)-1-oxoisoindolin-2-yl)piperidine-2,6-dione

To a solution of 6-(3-cyclopropyl-1-(trans-3-ethynylcyclobutyl)-1H-pyrazol-4-yl)-3-fluoro-2-methylpyridine (240 mg, 812.59 μmol) and 3-(5-bromo-1-oxo-isoindolin-2-yl)piperidine-2,6-dione (393.87 mg, 1.22 mmol) in DMSO (10 mL) were added Pd(dppf)Cl₂ (59.40 mg, 81.26 μmol), CuI (154.76 mg, 812.59 μmol) and TEA (822.26 mg, 8.13 mmol) at rt under N₂. The mixture was warmed to 90 [symbol] for 2 h, before it was purified by prep-HPLC and prep-TLC (DCM:MeOH=25:1) to afford the title compound (150 mg, yield: 34%) as a white solid. MS (ESI) m/z=538.7 [M+H]⁺.

### Example 113. 5-(((Trans-3-(3-cyclopropyl-4-(6-(1-methylazetidin-3-yl)quinoxalin-2-yl)-1H-pyrazol-1-yl)cyclobutyl)methyl)amino)-2-(2,6-dioxopiperidin-3-yl)isoindoline-1,3-dione (GS-755)

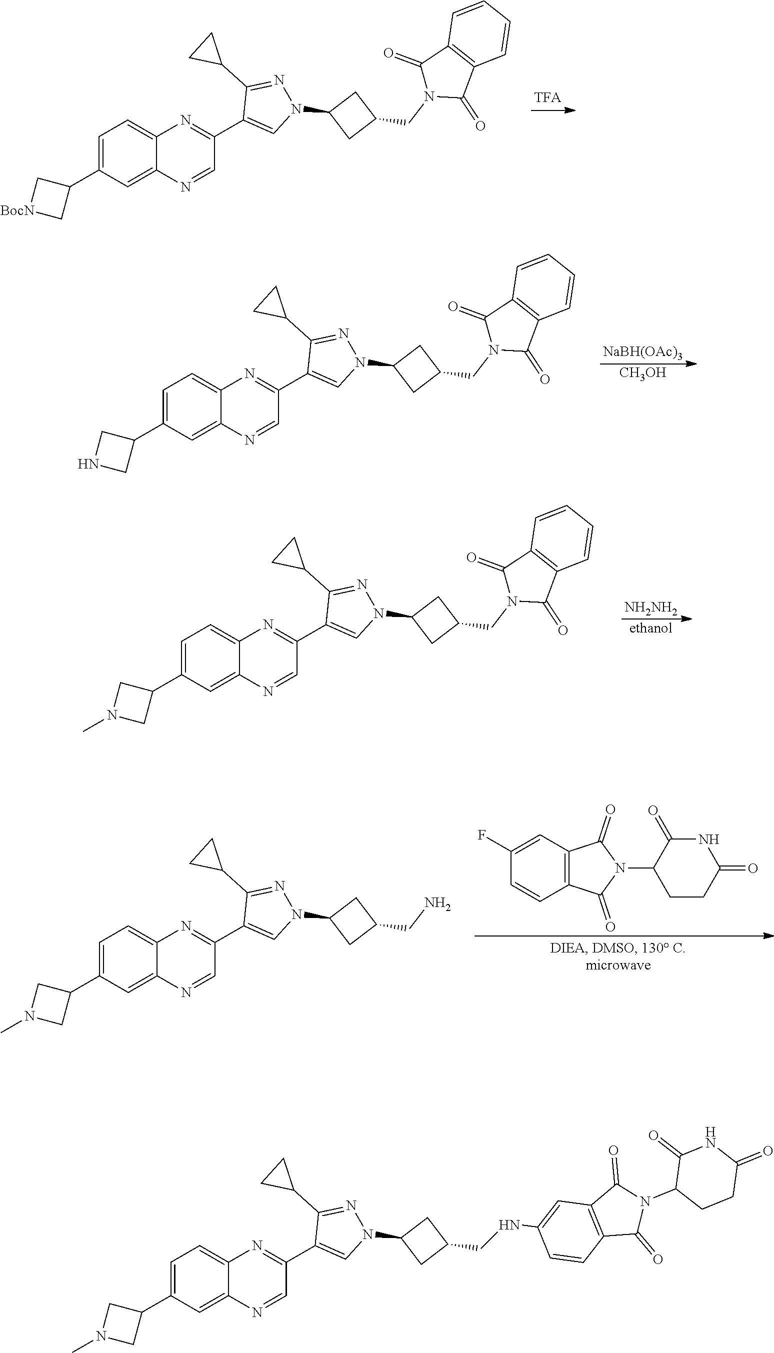

### Step 1. Synthesis of 2-((trans-3-(4-(6-(azetidin-3-yl)quinoxalin-2-yl)-3-cyclopropyl-1H-pyrazol-1-yl)cyclobutyl)methyl)isoindoline-1,3-dione

To a solution of tert-butyl 3-(2-(3-cyclopropyl-1-(trans-3-((1,3-dioxoisoindolin-2-yl)methyl)cyclobutyl)-1H-pyrazol-4-yl)quinoxalin-6-yl)azetidine-1-carboxylate (196 mg, 324.13 μmol) in DCM (1 mL) was added TFA (1 mL). The mixture was stirred at rt for 1 h, before it was concentrated. The residue was purified by reverse phase chromatography (0.10% TFA in water:MeOH=1:1) to afford the title compound (146 mg, yield: 89%) as a yellow oil. MS (ESI) m/z=505.3 [M+H]⁺.

### Step 2. Synthesis of 2-((trans-3-(3-cyclopropyl-4-(6-(1-methylazetidin-3-yl)quinoxalin-2-yl)-1H-pyrazol-1-yl)cyclobutyl)methyl)isoindoline-1,3-dione

To a solution of 2-((trans-3-(4-(6-(azetidin-3-yl)quinoxalin-2-yl)-3-cyclopropyl-1H-pyrazol-1-yl)cyclobutyl)methyl)isoindoline-1,3-dione (160 mg, 317.09 μmol) in formaldehyde solution (37 wt. % in H₂O, 2 mL, 26 mmol) was added NaBH(OAc)₃ (140.16 mg, 634.19 μmol). The mixture was stirred at rt for 30 min, before it was concentrated to afford the title compound (142 mg, yield: 86%) as a yellow oil, which was used directly in the next step. MS (ESI) m/z=519.2 [M+H]⁺.

### Step 3 to 4. Synthesis of 5-(((trans-3-(3-cyclopropyl-4-(6-(1-methylazetidin-3-yl)quinoxalin-2-yl)-1H-pyrazol-1-yl)cyclobutyl)methyl)amino)-2-(2,6-dioxopiperidin-3-yl)isoindoline-1,3-dione

GS-755 was synthesized following the procedures for steps 5 to 6 of GS-742 (3 mg, yield: 3% over 2 steps) as an off-white solid. MS (ESI) m/z=645.3 [M+H]⁺.

### Example 114. 5-(((Trans-3-(5-amino-3-cyclopropyl-1H-pyrazolo[3,4-b]pyridin-1-yl)cyclobutyl)methyl)amino)-2-(2,6-dioxopiperidin-3-yl)isoindoline-1,3-dione (GS-756)

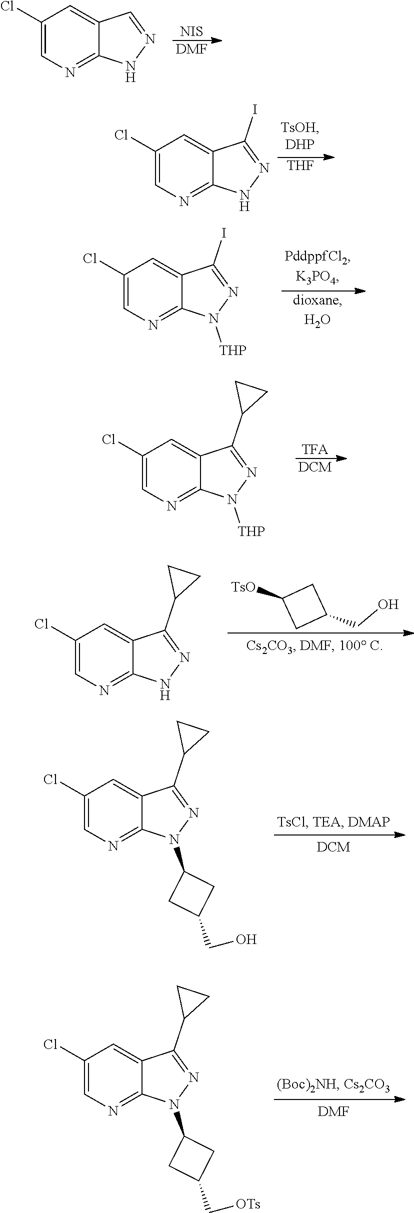

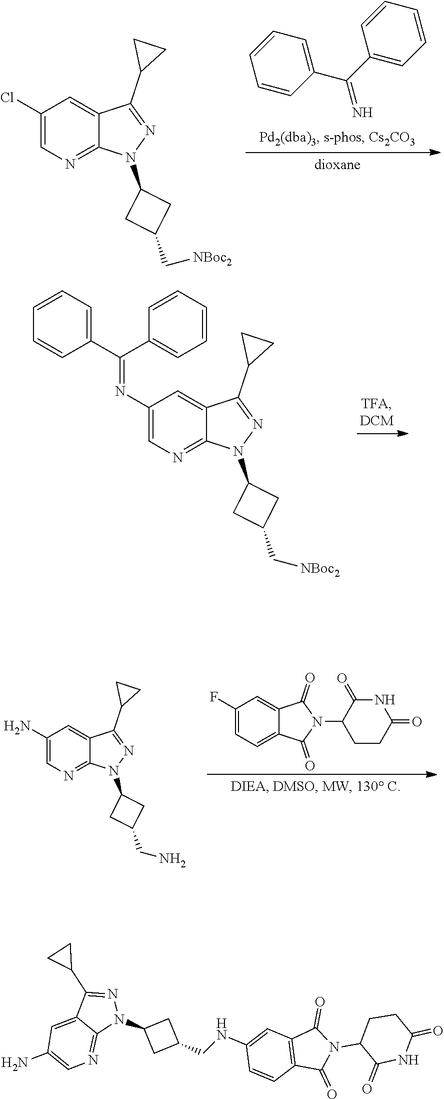

### Step 1. Synthesis of 5-chloro-3-iodo-1H-pyrazolo[3,4-b]pyridine

To a solution of 5-chloro-1H-pyrazolo[3,4-b]pyridine (5 g, 32.7 mmol) in DMF (80 mL) was added NIS (16.6 g, 65.4 mmol) at 0 [symbol] . The reaction mixture was stirred at rt for 3 h. H₂O (200 mL) was added. The mixture was filtered. The filter cake was washed with H₂O, and dried in vacuo at 45 [symbol] for 18 h to afford the title compound (6.3 g, yield: 69%) as a white solid. MS (ESI) m/z=280.47 [M+H]⁺.

### Step 2. Synthesis of 5-chloro-3-iodo-1-(tetrahydro-2H-pyran-2-yl)-1H-pyrazolo[3,4-b]pyridine

A mixture of 5-chloro-3-iodo-1H-pyrazolo[3,4-b]pyridine (3.0 g, 10.8 mmol), DHP (1.36 g, 16.2 mmol) and TsOH (371 mg, 2.16 mmol) in THF (30 mL) was stirred at rt for 15 h under N₂, before it was diluted with EtOAc, and washed with brine. The organic layer was concentrated. The residue was purified by silica gel chromatography to afford the title compound (2.8 g, yield: 71%) as a white solid. MS (ESI) m/z=364.58 [M+H]⁺.

### Step 3. Synthesis of 5-chloro-3-cyclopropyl-1-(tetrahydro-2H-pyran-2-yl)-H-pyrazolo[3,4-b]pyridine

A mixture of 5-chloro-3-iodo-1-(tetrahydro-2H-pyran-2-yl)-1H-pyrazolo[3,4-b]pyridine (2.8 g, 7.69 mmol), Pd(dppf)Cl₂ (623.2 mg, 0.770 mmol) and K₂CO₃ (3.18 g, 23.1 mmol) in dioxane (100 mL) and H₂O (10 mL) was stirred at 100 [symbol] for 15 h under N₂, before it was diluted with EtOAc and washed with brine. The organic layer was concentrated and the residue was purified by silica gel chromatography to afford the title compound (1.60 g, yield: 75%) as a white solid. MS (ESI) m/z=278.75 [M+H]⁺.

### Step 4. Synthesis of 5-chloro-3-cyclopropyl-1H-pyrazolo[3,4-b]pyridine

To a solution of 5-chloro-3-cyclopropyl-1-(tetrahydro-2H-pyran-2-yl)-1H-pyrazolo[3,4-b]pyridine (1.60 g, 5.76 mmol) in DCM (10 mL) was added TFA (5 mL). The mixture was stirred at rt for 30 min and concentrated to afford the title compound (1.01 g, yield: 91%) as a yellow solid. MS (ESI) m/z=194.63 [M+H]⁺.

### Step 5. Synthesis of (trans-3-(5-chloro-3-cyclopropyl-1H-pyrazolo[3,4-b]pyridin-1-yl)cyclobutyl)methanol

To a solution of 5-chloro-3-cyclopropyl-1H-pyrazolo[3,4-b]pyridine (1.01 g, 5.22 mmol) and cis-3-(hydroxymethyl)cyclobutyl 4-methylbenzenesulfonate (2.0 g, 7.83 mmol) in DMSO (10 mL) was added Cs₂CO₃ (5.12 g, 15.6 mmol). The mixture was stirred at 95 [symbol] for 1 h, before it was diluted with EtOAc (50 mL) and washed with H₂O and brine. The organic layer was concentrated and the residue was purified by silica gel chromatography to afford the title compound (900 mg, yield: 62%) as a yellow solid. MS (ESI) m/z=278.75 [M+H]⁺.

### Step 6. Synthesis of (trans-3-(5-chloro-3-cyclopropyl-1H-pyrazolo[3,4-b]pyridin-1-yl)cyclobutyl)methyl 4-methylbenzenesulfonate

To a solution of (trans-3-(5-chloro-3-cyclopropyl-1H-pyrazolo[3,4-b]pyridin-1-yl)cyclobutyl)methanol (900 mg, 3.24 mmol) and TsCl (922 mg, 4.86 mmol) in DCM (10 mL) were added DMAP (39 mg, 0.324 μmol) and TEA (0.98 g, 9.7 mmol). After stirring at rt for 3 h, the mixture was diluted with DCM (20 mL), and washed with H₂O and brine. The organic layer was concentrated, and the residue was purified by silica gel chromatography to afford the title compound (750 mg, yield: 54%) as a colorless oil. MS (ESI) m/z=432.94 [M+H]⁺.

### Step 7. Synthesis of tert-butyl (tert-butoxycarbonyl)((trans-3-(5-chloro-3-cyclopropyl-1H-pyrazolo[3,4-b]pyridin-1-yl)cyclobutyl)methyl)carbamate

To a solution of (trans-3-(5-chloro-3-cyclopropyl-1H-pyrazolo[3,4-b]pyridin-1-yl)cyclobutyl)methyl 4-methylbenzenesulfonate (0.75 g, 1.73 mmol) and di-tert-butyl iminodicarbonate (0.75 g, 3.46 mmol) in DMF (10 mL) was added Cs₂CO₃ (1.69 g, 5.19 mmol). The reaction mixture was stirred at 100° C. for 2 h, before it was concentrated. The residue was purified by reverse phase chromatography to afford the title compound (658 mg, yield: 78%) as a white solid. MS (ESI) m/z=478.0 [M+H]⁺.

### Step 8. Synthesis of tert-butyl (tert-butoxycarbonyl)((trans-3-(3-cyclopropyl-5-((diphenylmethylene)amino)-1H-pyrazolo[3,4-b]pyridin-1-yl)cyclobutyl)methyl)carbamate

To a solution of tert-butyl (tert-butoxycarbonyl)((trans-3-(5-chloro-3-cyclopropyl-1H-pyrazolo[3,4-b]pyridin-1-yl)cyclobutyl)methyl)carbamate (658 mg, 1.38 mmol) and diphenylmethanimine (500 mg, 2.76 mmol) in dioxane (10 mL) were added Pd₂(dba)₃ (126 mg, 0.138 mmol), S-phos (113 mg, 0.276 mmol) and Cs₂CO₃ (1.34 g, 4.14 mmol). The reaction mixture was stirred at 100 [symbol] for 2 h under N₂, before it was diluted with water and extracted with EtOAc three times. The combined organic layers were washed with brine, dried over anhydrous Na₂SO₄, filtered and concentrated under reduced pressure. The residue was purified by silica gel chromatography to afford the title compound (497 mg, yield: 58%) as a white solid. MS (ESI) m/z=622.78 [M+H]⁺.

### Step 9. Synthesis of 1-(trans-3-(aminomethyl)cyclobutyl)-3-cyclopropyl-1H-pyrazolo[3,4-b]pyridin-5-amine

To a solution of tert-butyl (tert-butoxycarbonyl)((trans-3-(3-cyclopropyl-5-((diphenylmethylene)amino)-1H-pyrazolo[3,4-b]pyridin-1-yl)cyclobutyl)methyl)carbamate (497 mg, 0.8 mmol) in DCM (4 mL) was added TFA (2 mL). The reaction mixture was stirred at rt for 1 h, before it was concentrated. The residue was purified by reverse phase chromatography to afford the title compound (180 mg, yield: 87%) as a white solid. MS (ESI) m/z=258.34 [M+H]⁺.

### Step 10. Synthesis of 5-(((trans-3-(5-amino-3-cyclopropyl-1H-pyrazolo[3,4-b]pyridin-1-yl)cyclobutyl)methyl)amino)-2-(2,6-dioxopiperidin-3-yl)isoindoline-1,3-dione

To a solution of 1-(trans-3-(aminomethyl)cyclobutyl)-3-cyclopropyl-1H-pyrazolo[3,4-b]pyridin-5-amine (50 mg, 194 μmol) in DMSO (5 mL) were added 2-(2,6-dioxo-3-piperidyl)-5-fluoro-isoindoline-1,3-dione (58.8 mg, 213 μmol) and KF (112 mg, 1.94 mmol). After the mixture was stirred at 130 [symbol] for 30 min under microwave irradiation, it was purified by reverse phase chromatography to afford the title compound (14 mg, yield: 14%) as a yellow solid. MS (ESI) m/z=513.56 [M+H]⁺.

### Example 115. 5-(((Trans-3-(3-cyclopropyl-4-(piperazin-1-yl)-1H-indazol-1-yl)cyclobutyl)methyl)amino)-2-(2,6-dioxopiperidin-3-yl)isoindoline-1,3-dione (GS-757)

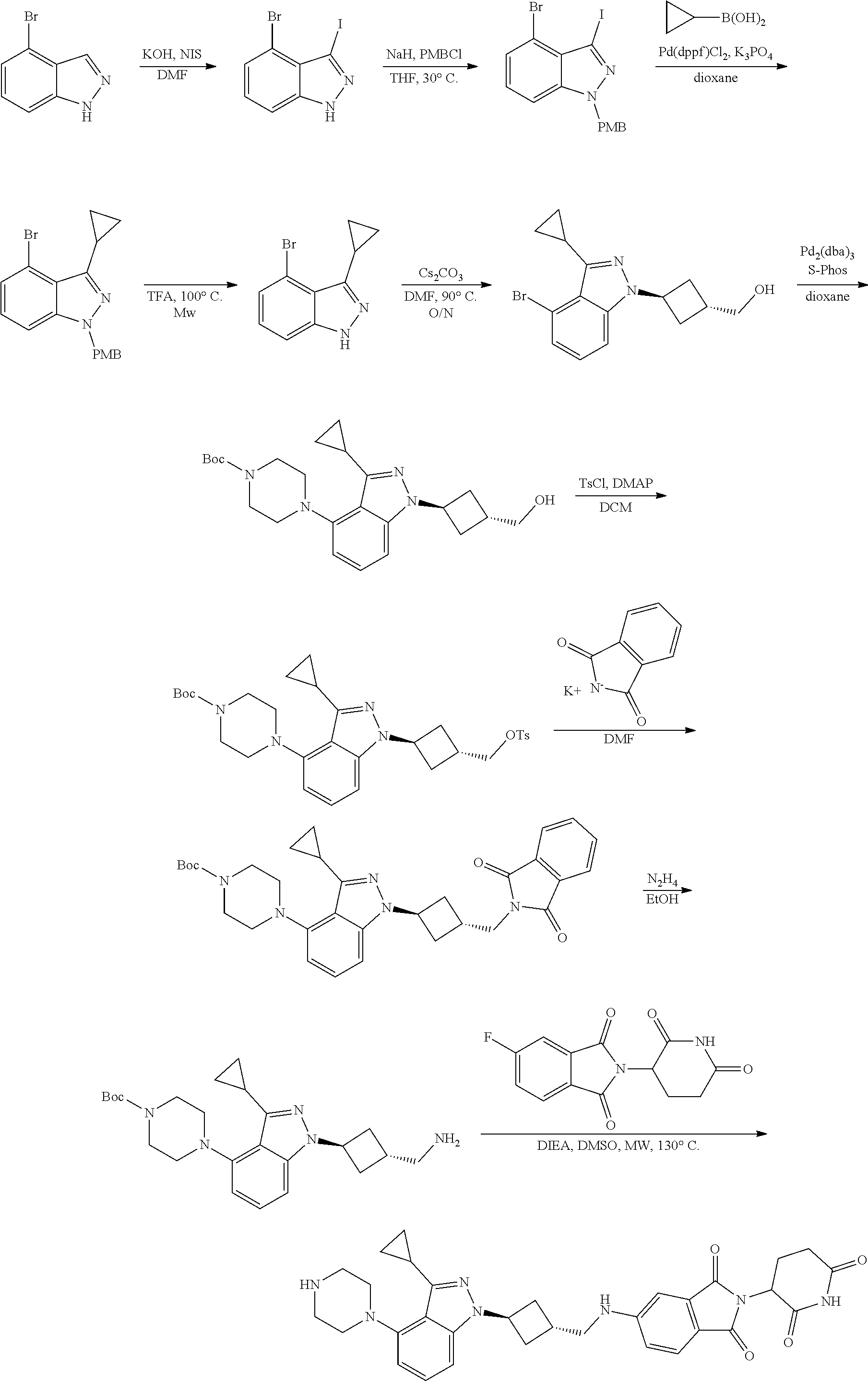

### Step 1. Synthesis of 4-bromo-3-iodo-1H-indazole

A solution of 4-bromo-1H-indazole (10 g, 50.7 mmol) in DMF (100 mL) was added KOH (5.7 g, 101 mmol). After the reaction was stirred at rt for 20 min, NIS (12.5 g, 55.8 mmol) was added. The mixture was stirred at rt overnight before it was quenched with water. The mixture was extracted with EtOAc. The organic layer was washed with water and brine, dried over Na₂SO₄, filtered and concentrated to afford the title compound (14.4 g, yield: 88%) as a white solid. MS (ESI) m/z=325.1 [M+H]⁺.

### Step 2. Synthesis of 4-bromo-3-iodo-1-(4-methoxybenzyl)-1H-indazole

To a solution of 4-bromo-3-iodo-1H-indazole (14.4 g, 44.6 mmol) in THF (150 mL) was added NaH (1.61 g, 66.9 mmol) at 0 [symbol] under Ar. After the reaction was stirred at this temperature for 30 min, PMBCl (8.4 g, 53.5 mmol) was added. The mixture was then stirred at 40 [symbol] overnight, quenched with water, and extracted with EtOAc. The organic layer was washed with water and brine, dried over Na₂SO₄, filtered and concentrated. The residue was purified by silica gel chromatography (petroleum ether:EtOAc=10:0 to 9:1) to afford the title compound (14 g, yield: 710%) as a white solid. MS (ESI) m/z=445.1 [M+H]⁺.

### Step 3. Synthesis of 4-bromo-3-cyclopropyl-1-(4-methoxybenzyl)-1H-indazole

To a mixture of 4-bromo-3-iodo-1-(4-methoxybenzyl)-1H-indazole (14 g, 31.6 mmol) and cyclopropylboronic acid (8.1 g, 94.8 mmol) in dioxane (150 mL) were added Pd(dppf)Cl₂ (2.2 g, 3.2 mmol) and K₃PO₄ (13.4 g, 63.2 mmol). After the reaction mixture was stirred at 90 [symbol] overnight, it was quenched with water, and extracted with EtOAc. The organic layer was washed with water and brine, dried over Na₂SO₄, filtered and concentrated. The residue was purified by silica gel chromatography (petroleum ether:EtOAc=10:0 to 9:1) to afford the title compound (5.5 g, yield: 49%) as a white solid. MS (ESI) m/z=359.2 [M+H]⁺.

### Step 4. Synthesis of 4-bromo-3-cyclopropyl-1H-indazole

A solution of 4-bromo-3-cyclopropyl-1-(4-methoxybenzyl)-1H-indazole (5.5 g, 15.4 mmol) in TFA (5 mL) was stirred at 100 [symbol] for 1 h under microwave irradiation. The mixture was concentrated, diluted with aq. NaHCO₃ solution, and extracted with EtOAc. The organic layer was washed with water and brine, dried over Na₂SO₄, filtered and concentrated. The residue was purified by silica gel chromatography (petroleum ether:EtOAc=10:0 to 7:3) to afford the title compound (3.3 g, yield: 90%) as a white solid. MS (ESI) m/z=239.0 [M+H]⁺.

### Step 5. Synthesis of (trans-3-(4-bromo-3-cyclopropyl-1H-indazol-1-yl)cyclobutyl)methanol

To a mixture of 4-bromo-3-cyclopropyl-1H-indazole (3.3 g, 13.92 mmol) and cis-3-(hydroxymethyl)cyclobutyl 4-methylbenzenesulfonate (5.4 g, 20.9 mmol) in DMSO (60 mL) was added Cs₂CO₃ (9.1 g, 27.8 mmol). After the reaction mixture was stirred at 90 [symbol] for 4 h, it was quenched with water, and extracted with EtOAc. The organic layer was washed with water and brine, dried over Na₂SO₄, filtered and concentrated. The residue was purified by reverse phase chromatography (0-70% MeCN in H₂O) to afford the title compound (2.3 g, yield: 52%) as a brown solid. MS (ESI) m/z=323.0 [M+H]⁺.

### Step 6. Synthesis of tert-butyl 4-(3-cyclopropyl-1-(trans-3-(hydroxymethyl)cyclobutyl)-1H-indazol-4-yl)piperazine-1-carboxylate

To a mixture of (trans-3-(4-bromo-3-cyclopropyl-1H-indazol-1-yl)cyclobutyl)methanol (2.3 g, 7.2 mmol) and tert-butyl piperazine-1-carboxylate (2.7 g, 14.3 mmol) in dioxane (50 mL) were added Pd₂(dba)₃ (656 mg, 716 μmol), S-phos (587 mg, 1.4 mmol) and Cs₂CO₃ (4.7 g, 14 mmol). After the reaction mixture was stirred at 90 [symbol] overnight, it was concentrated, diluted with water and extracted with EtOAc. The organic layer was washed with water and brine, dried over Na₂SO₄, filtered and concentrated. The residue was purified by reverse phase chromatography (0-70% MeCN in H₂O) to afford the title compound (450 mg, yield: 15%) as a brown solid. MS (ESI) m/z=427.3 [M+H]⁺.

### Step 7. Synthesis of tert-butyl 4-(3-cyclopropyl-1-(trans-3-((tosyloxy)methyl)cyclobutyl)-1H-indazol-4-yl)piperazine-1-carboxylate

To a solution of tert-butyl 4-(3-cyclopropyl-1-(trans-3-(hydroxymethyl)cyclobutyl)-1H-indazol-4-yl)piperazine-1-carboxylate (450 mg, 1.1 mmol) in DCM (10 mL) were added TsCl (402 mg, 2.1 mmol) and DMAP (13 mg, 105 μmol). The mixture was stirred at rt overnight before it was concentrated. The residue was purified by silica gel chromatography (DCM:MeOH=10:0 to 9:1) to afford the title compound (230 mg, yield: 38%) as a yellow solid. MS (ESI) m/z=581.3 [M+H]⁺.

### Step 8. Synthesis of tert-butyl 4-(3-cyclopropyl-1-(trans-3-((1,3-dioxoisoindolin-2-yl)methyl)cyclobutyl)-1H-indazol-4-yl)piperazine-1-carboxylate

To a solution of tert-butyl 4-(3-cyclopropyl-1-(trans-3-((tosyloxy)methyl)cyclobutyl)-1H-indazol-4-yl)piperazine-1-carboxylate (360 mg, 620 μmol) in DMF (10 mL) was added (1,3-dioxoisoindolin-2-yl)potassium (115 mg, 620 μmol). The mixture was stirred at 50° C. for 30 min, before it was purified by reverse phase chromatography (0-70% MeCN in H₂O) to afford the title compound (210 mg, yield: 61%) as a white solid. MS (ESI) m/z=556.3 [M+H]⁺.

### Step 9. Synthesis of tert-butyl 4-(1-(trans-3-(aminomethyl)cyclobutyl)-3-cyclopropyl-1H-indazol-4-yl)piperazine-1-carboxylate

To a solution of tert-butyl 4-(3-cyclopropyl-1-(trans-3-((1,3-dioxoisoindolin-2-yl)methyl)cyclobutyl)-11H-indazol-4-yl)piperazine-1-carboxylate (210 mg, 378 μmol) in EtOH (5 mL) was added N₂H₄ (121 mg, 3.9 mmol). The mixture was stirred at rt for 30 min, before it was concentrated. The residue was purified by reverse phase chromatography (0-70% MeCN in H₂O) to afford the title compound (100 mg, yield: 62%) as a white solid. MS (ESI) m/z=426.4 [M+H]⁺.

### Step 10. Synthesis of 5-(((trans-3-(3-cyclopropyl-4-(piperazin-1-yl)-1H-indazol-1-yl)cyclobutyl)methyl)amino)-2-(2.6-dioxopiperidin-3-yl)isoindoline-1,3-dione

To a mixture of tert-butyl 4-(1-(trans-3-(aminomethyl)cyclobutyl)-3-cyclopropyl-1H-indazol-4-yl)piperazine-1-carboxylate (45 mg, 106 μmol) and 2-(2,6-dioxo-3-piperidyl)-5-fluoro-isoindoline-1,3-dione (44 mg, 159 μmol) in DMSO (1 mL) was added DIPEA (41 mg, 317 μmol). After the mixture was stirred at 130 [symbol] for 2 h under microwave irradiation, it was purified by reverse phase chromatography (0-70% MeCN in H₂O) to afford the title compound (5.3 mg, yield: 9%) as a green solid. MS (ESI) m/z=582.6 [M+H]⁺.

### Example 116. 3-(5-(2-(Trans-3-(3-cyclopropyl-4-(5-fluoro-6-methylpyridin-2-yl)-1H-pyrazol-1-yl)cyclobutyl)ethyl)-1-oxoisoindolin-2-yl)piperidine-2,6-dione (GS-758)

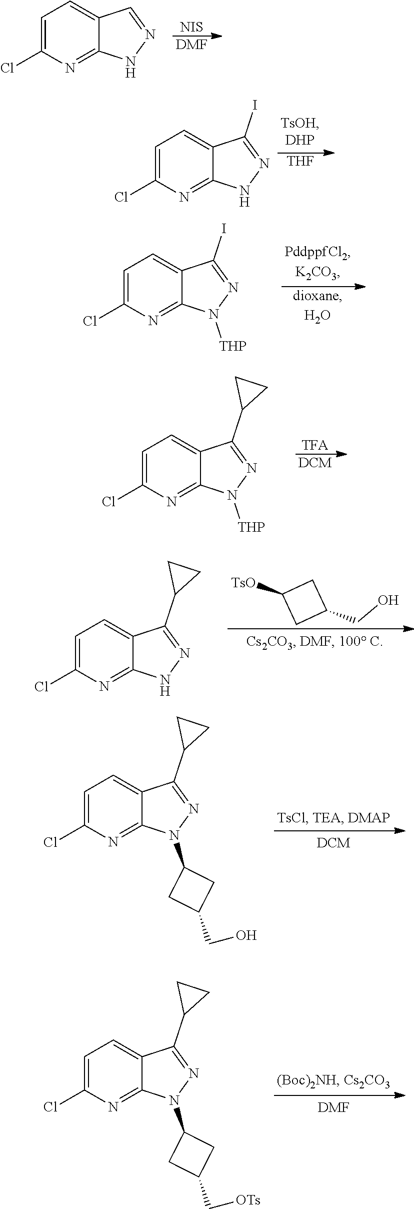

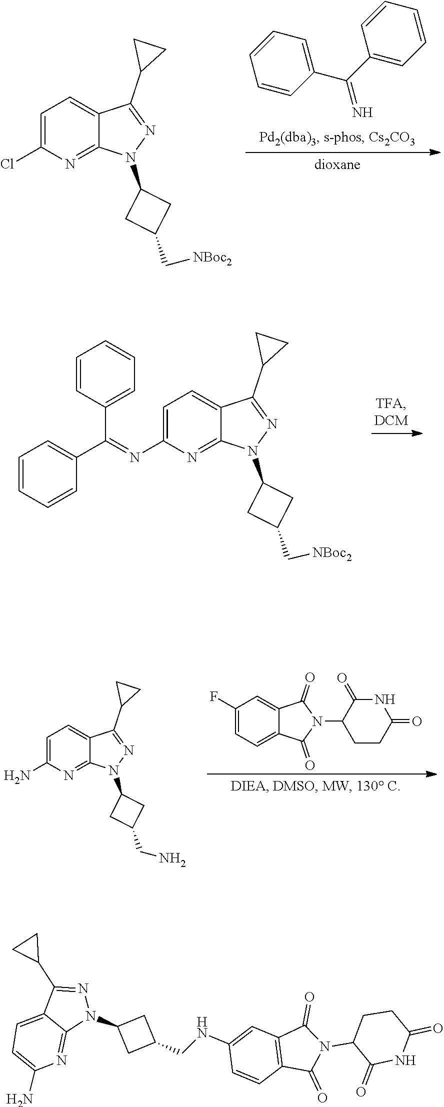

GS-758 was synthesized following the procedures for GS-756 (16 mg, yield: 1% over 10 steps) as a yellow solid. MS (ESI) m/z=513.56 [M+H]⁺.

Examples 117 and 118. 5-(((Cis-3-(3-cyclopropyl-4-(5-fluoro-6-methylpyridin-2-yl)-1H-pyrazol-1-yl)-1-hydroxycyclobutyl)methyl)amino)-2-(2,6-dioxopiperidin-3-yl)isoindoline-1,3-dione (GS-759) and 5-(((trans-3-(3-cyclopropyl-4-(5-fluoro-6-methylpyridin-2-yl)-1H-pyrazol-1-yl)-1-hydroxycyclobutyl)methyl)amino)-2-(2,6-dioxopiperidin-3-yl)isoindoline-1,3-dione (GS-760)

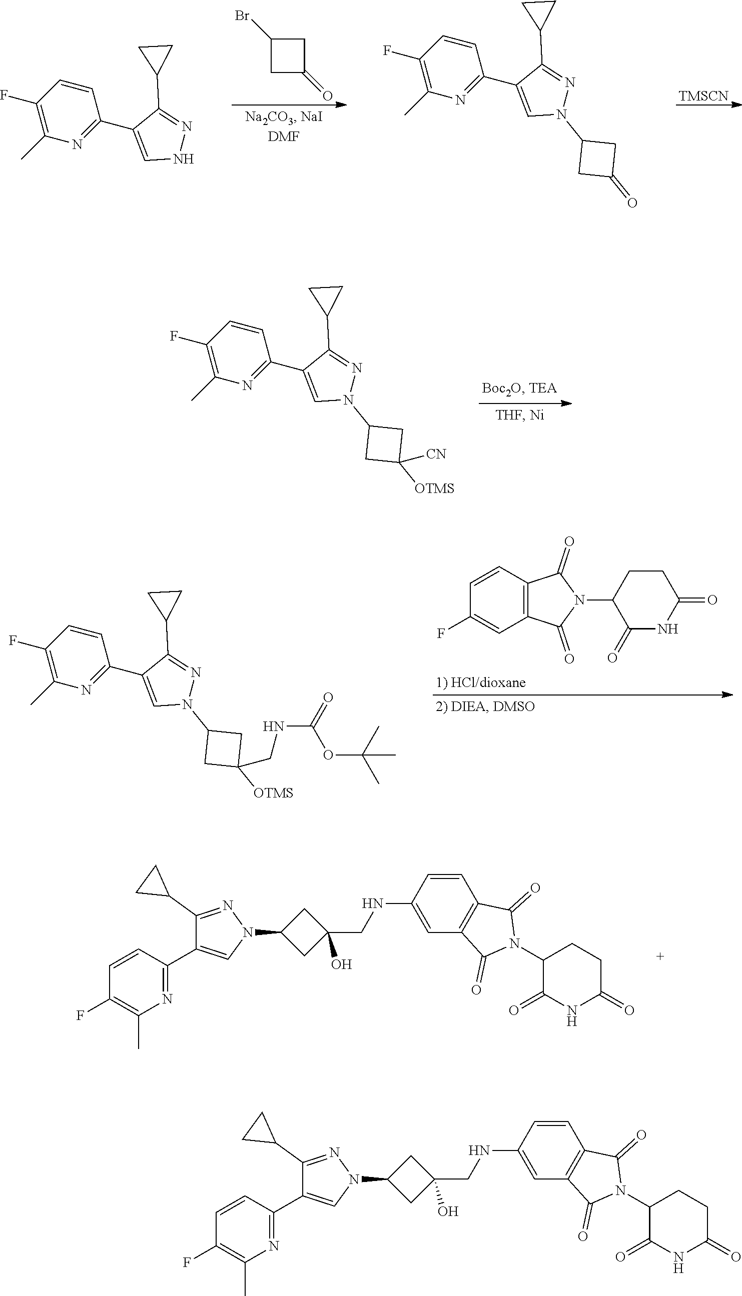

### Step 1. Synthesis of 3-(3-cyclopropyl-4-(5-fluoro-6-methylpyridin-2-yl)-1H-pyrazol-1-yl)cyclobutan-1-one

A mixture of 6-(3-cyclopropyl-1H-pyrazol-4-yl)-3-fluoro-2-methylpyridine (200 mg, 883.81 μmol), 3-bromocyclobutanone (250 mg, 1.68 mmol), Na₂CO₃ (200 mg, 1.89 mmol) and NaI (20 mg, 133.43 μmol) in DMF (2 mL) was heated at 80 [symbol] for 0.5 h. The mixture was cooled to rt, before it was purified by reverse phase chromatography (ACN in 0.05% TFA solution) to afford the title compound (80 mg, yield: 29%) as a light yellow solid. ¹H NMR (400 MHz, CDCl₃) δ 8.51 (s, 1H), 8.04-7.95 (m, 2H), 5.05-5.01 (m, 1H), 3.77-3.71 (m, 2H), 3.59-3.52 (m, 2H), 2.81 (s, 3H), 1.87-1.83 (m, 1H), 1.03-1.01 (m, 4H).

### Step 2. Synthesis of 3-(3-cyclopropyl-4-(5-fluoro-6-methylpyridin-2-yl)-1H-pyrazol-1-yl)-1-((trimethylsilyl)oxy)cyclobutane-1-carbonitrile

To a solution of 3-(3-cyclopropyl-4-(5-fluoro-6-methylpyridin-2-yl)-1H-pyrazol-1-yl)cyclobutan-1-one (80 mg, 252.35 μmol) in DCM (8 mL) were added TMSCN (80 mg, 806.37 μmol) and NMO (20 mg, 644.87 μmol) at rt. The mixture was stirred overnight, before it was concentrated to afford the title compound (crude, 100 mg, yield: 62%) as a light brown oil. MS (ESI) m/z=385.5 [M+H]⁺.

### Step 3. Synthesis of tert-butyl ((3-(3-cyclopropyl-4-(5-fluoro-6-methylpyridin-2-yl)-1H-pyrazol-1-yl)-1-hydroxycyclobutyl)methyl)carbamate

A mixture of 3-(3-cyclopropyl-4-(5-fluoro-6-methylpyridin-2-yl)-1H-pyrazol-1-yl)-1-((trimethylsilyl)oxy)cyclobutane-1-carbonitrile (100 mg, 156.04 μmol), tert-butoxycarbonyl tert-butyl carbonate (34.05 mg, 156.04 μmol), TEA (50 mg, 494.12 μmol) and Raney-Nickel (20 mg, 340.75 μmol) in THF (10 mL) was stirred at rt under balloon pressure of H₂ overnight. The mixture was filtered and concentrated. The residue was purified by reverse phase chromatography (ACN in 0.05% TFA solution) to afford the title compound (10 mg, yield: 12%) as a colorless oil. MS (ESI) m/z=417.3 [M+H]⁺.

### Step 4. Synthesis of 5-(((cis-3-(3-cyclopropyl-4-(5-fluoro-6-methylpyridin-2-yl)-1H-pyrazol-1-yl)-1-hydroxycyclobutyl)methyl)amino)-2-(2,6-dioxopiperidin-3-yl)isoindoline-1,3-dione and 5-(((trans-3-(3-cyclopropyl-4-(5-fluoro-6-methylpyridin-2-yl)-1H-pyrazol-1-yl)-1-hydroxycyclobutyl)methyl)amino)-2-(2.6-dioxopiperidin-3-yl)isoindoline-1,3-dione

GS-759 and GS-760 were synthesized following the procedures for steps 6 to 7 of GS-736. The mixture was purified by prep-HPLC to afford the title compounds.

5-(((Cis-3-(3-cyclopropyl-4-(5-fluoro-6-methylpyridin-2-yl)-1H-pyrazol-1-yl)-1-hydroxycyclobutyl)methyl)amino)-2-(2,6-dioxopiperidin-3-yl)isoindoline-1,3-dione (2.4 mg, yield: 22%) as a yellow solid. MS (ESI) m/z=573.5 [M+H]⁺.

5-(((Trans-3-(3-cyclopropyl-4-(5-fluoro-6-methylpyridin-2-yl)-1H-pyrazol-1-yl)-1-hydroxycyclobutyl)methyl)amino)-2-(2,6-dioxopiperidin-3-yl)isoindoline-1,3-dione (2.2 mg, yield: 20%) as a yellow solid. MS (ESI) m/z=573.5 [M+H]⁺.

### Example 119. 5-((Trans-3-(3-cyclopropyl-4-(1-methyl-1H-pyrazolo[4,3-c]pyridin-6-yl)-1H-pyrazol-1-yl)cyclobutyl)methoxy)-2-(2,6-dioxopiperidin-3-yl)isoindoline-1,3-dione (GS-761)

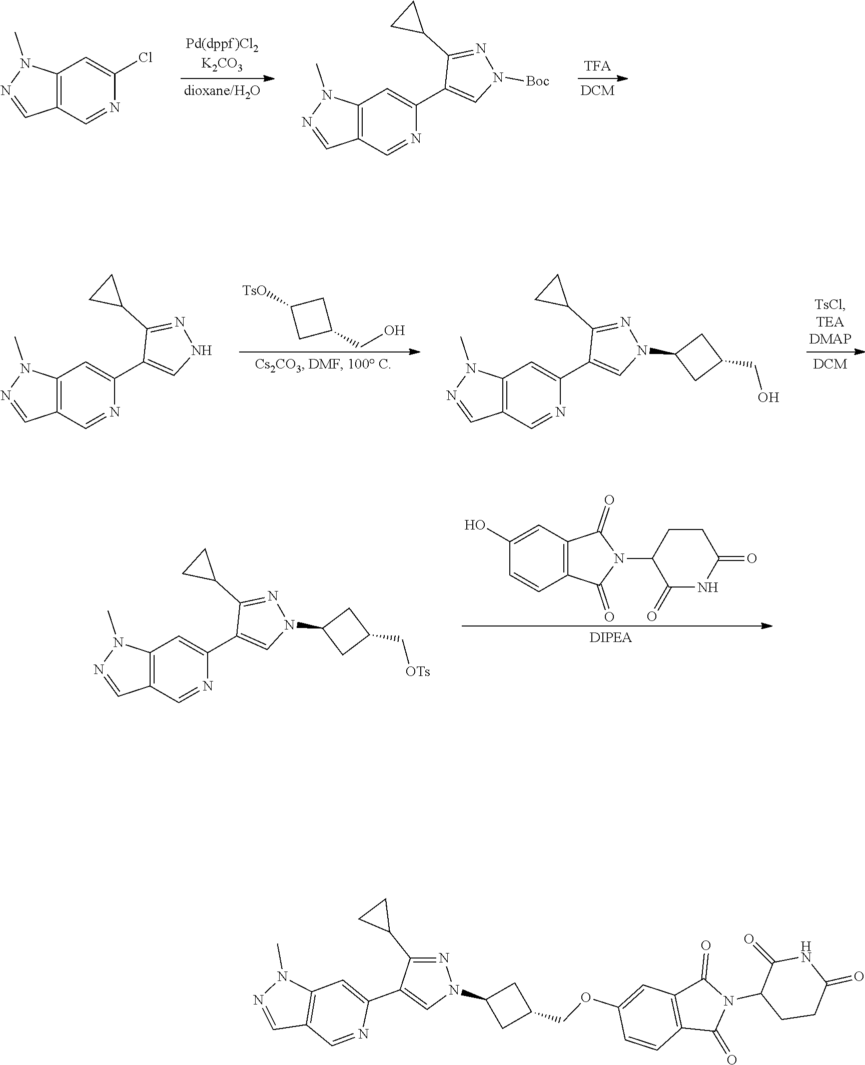

### Step 1. Synthesis of tert-butyl 3-cyclopropyl-4-(1-methyl-1H-pyrazolo[4,3-c]pyridin-6-yl)-1H-pyrazole-1-carboxylate

The title compound was synthesized following the procedure as described for step 1 of GS-748 (2.0 g, yield: 79%). MS (ESI) m/z=340.2 [M+H]⁺.

### Step 2 to 4. Synthesis of (trans-3-(3-cyclopropyl-4-(1-methyl-1H-pyrazolo[4,3-c]pyridin-6-yl)-1H-pyrazol-1-yl)cyclobutyl)methyl 4-methylbenzenesulfonate

The title compound was synthesized following the procedures for steps 2 to 4 of GS-717 (70 mg, yield: 16%) as a gray solid. MS (ESI) m/z=478.2 [M+H]⁺.

### Step 5. Synthesis of 5-((trans-3-(3-cyclopropyl-4-(1-methyl-1H-pyrazolo[4,3-c]pyridin-6-y)-1H-pyrazol-1-yl)cyclobutyl)methoxy)-2-(2,6-dioxopiperidin-3-yl)isoindoline-1,3-dione

To a mixture of (trans-3-(3-cyclopropyl-4-(1-methyl-1H-pyrazolo[4,3-c]pyridin-6-yl)-1H-pyrazol-1-yl)cyclobutyl)methyl 4-methylbenzenesulfonate (20 mg, 41.9 μmol) and 2-(2,6-dioxo-3-piperidyl)-5-hydroxy-isoindoline-1,3-dione (11 mg, 41.9 μmol) in DMSO (1 mL) was added DIPEA (16 mg, 125 μmol). After the reaction was stirred at 80 [symbol] overnight, it was purified by reverse flash chromatography (ACN in 0.05% TFA solution, 0-70%) to afford the title compound (2.3 mg, yield: 10%) as a white solid. MS (ESI) m/z=580.5 [M+H]⁺.

### Example 120. 5-(((Trans-3-(4-amino-3-cyclopropyl-1H-pyrazolo[3,4-b]pyridin-1-yl)cyclobutyl)methyl)amino)-2-(2,6-dioxopiperidin-3-yl)isoindoline-1,3-dione (GS-762)

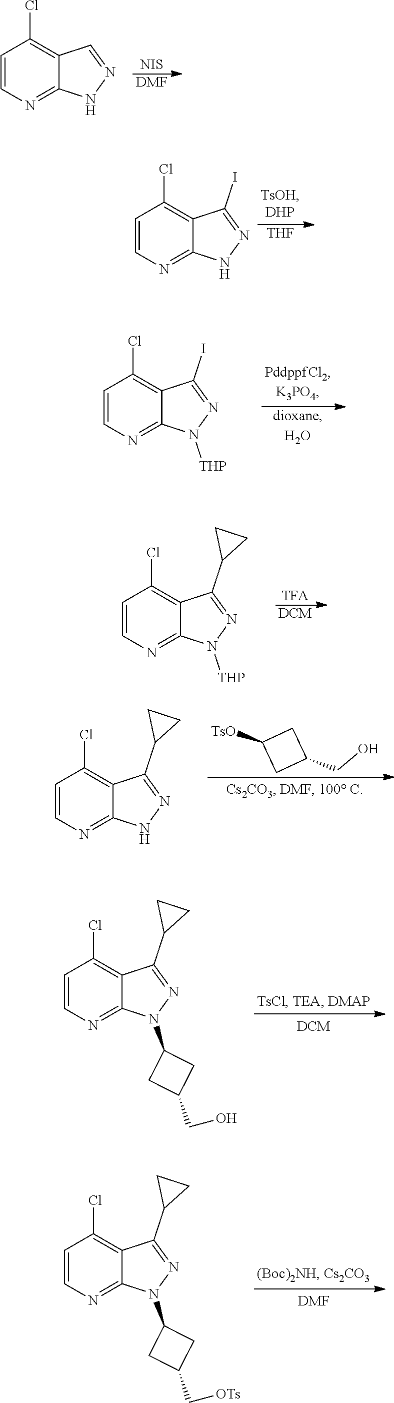

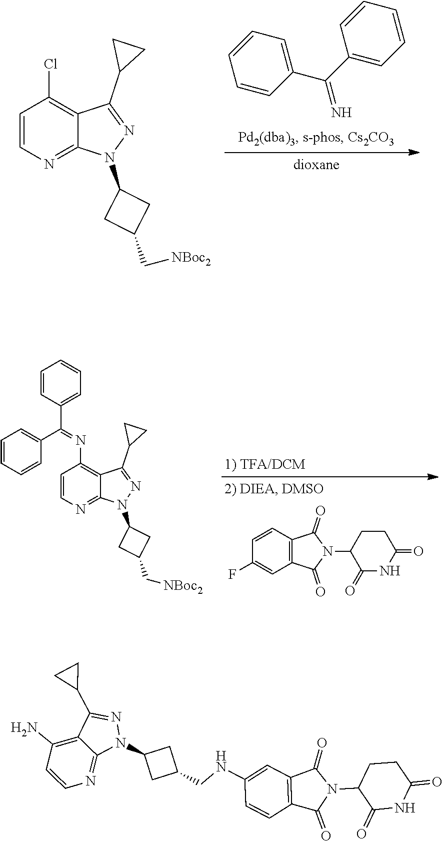

GS-762 was synthesized following the procedures for GS-756 (17 mg, yield: 0.6% over 10 steps) as a yellow solid. MS (ESI) m/z=513.56 [M+H]⁺.

### Example 121. 5-(((Trans-3-(3-cyclopropyl-4-(1-(3-hydroxycyclobutyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-763)

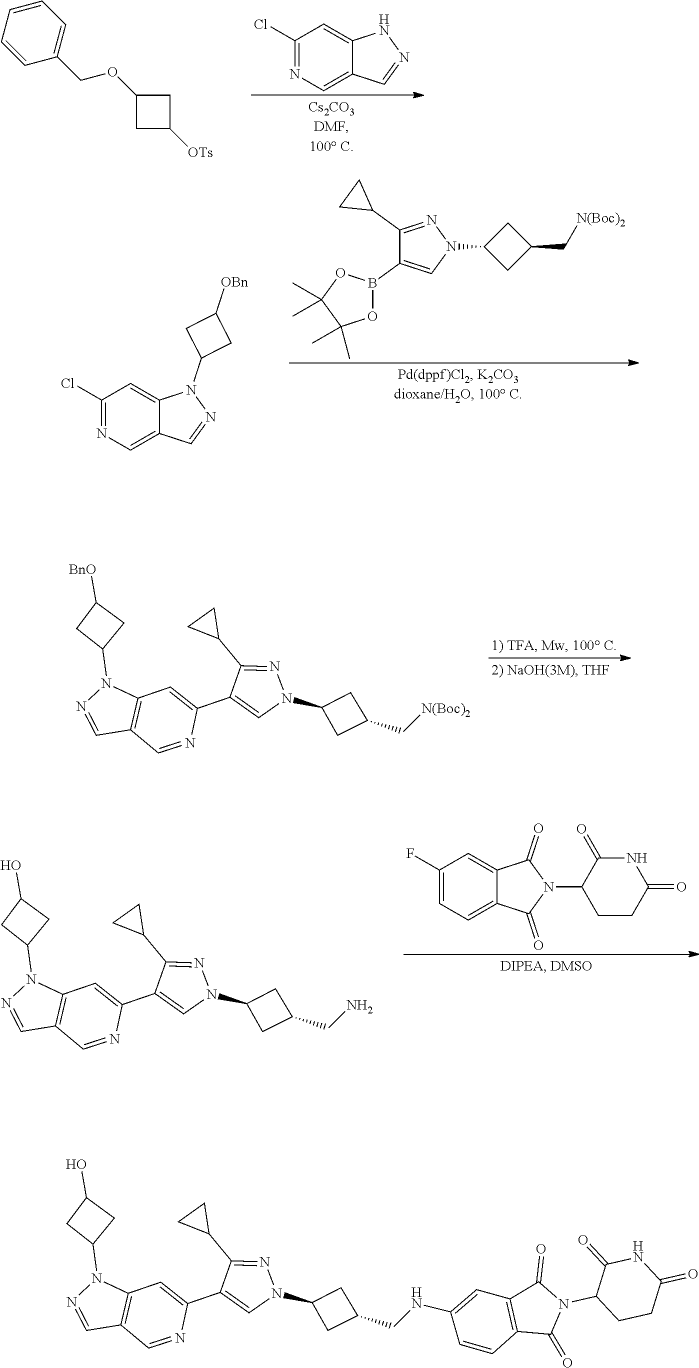

### Step 1. Synthesis of 1-(3-(benzyloxy)cyclobutyl)-6-chloro-1H-pyrazolo[4,3-c]pyridine

To a solution of (3-benzyloxycyclobutyl) 4-methylbenzenesulfonate (500 mg, 1.50 mmol) in DMF (4 mL) were added Cs₂CO₃ (735.12 mg, 2.26 mmol) and 6-chloro-11H-pyrazolo[4,3-c]pyridine (254.09 mg, 1.65 mmol). The mixture was stirred at 100 [symbol] for 1 h, before it was purified by silica gel chromatograph (petroleum ether:EtOAc=1:0 to 3:1) to afford the title compound (300 mg, yield: 63%). MS (ESI) m/z=314.13 [M+H]⁺.

### Step 2. Synthesis of di-tert-butyl ((trans-3-(4-(1-(3-(benzyloxy)cyclobutyl)-1H-pyrazolo[4,3-c]pyridin-6-yl)-3-cyclopropyl-1H-pyrazol-1-yl)cyclobutyl)methyl)iminodicarbonate

To a solution of 1-(3-(benzyloxy)cyclobutyl)-6-chloro-1H-pyrazolo[4,3-c]pyridine (160 mg, 509.91 μmol) in dioxane (1.5 mL) and H₂O (0.3 mL) were added tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (290.25 mg, 560.90 μmol) and K₂CO₃ (140.95 mg, 1.02 mmol). The mixture was purged with N₂ three times, before Pd(dppf)Cl₂ (40 mg, 50.99 μmol) was added under N₂. The mixture was stirred at 100 [symbol] for 12 h, before it was filtered. The filtrate was concentrated under reduced pressure. The residue was purified by silica gel chromatography (petroleum ether:EtOAc=1:0 to 3:1) to afford the title compound (60 mg, yield: 17%). MS (ESI) m/z=669.42 [M+H]⁺.

### Step 3. Synthesis of 3-(6-(1-(trans-3-(aminomethyl)cyclobutyl)-3-cyclopropyl-1H-pyrazol-4-yl)-1H-pyrazolo[4,3-c]pyridin-1-yl)cyclobutan-1-ol

A solution of di-tert-butyl ((trans-3-(4-(1-(3-(benzyloxy)cyclobutyl)-1H-pyrazolo[4,3-c]pyridin-6-yl)-3-cyclopropyl-1H-pyrazol-1-yl)cyclobutyl)methyl)iminodicarbonate (60 mg, 89.71 μmol) in TFA (6.82 mg, 2 mL) was stirred at 100 [symbol] for 0.5 h under microwave irradiation, before it was concentrated under reduced pressure. The residue was dissolved in THF (2 mL). NaOH (2.5 M, 253 μL) was added. The mixture was stirred at 20 [symbol] for 0.5 h, before it was concentrated under reduced pressure. The residue was purified by reverse phase chromatography (ACN in 0.05% TFA solution, 0-75%) to afford the title compound (28 mg, yield: 82%). MS (ESI) m/z=379.27 [M+H]⁺.

### Step 4. Synthesis of 5-(((trans-3-(3-cyclopropyl-4-(1-(3-hydroxycyclobutyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

To a solution of 3-(6-(1-(trans-3-(aminomethyl)cyclobutyl)-3-cyclopropyl-1H-pyrazol-4-yl)-1H-pyrazolo[4,3-c]pyridin-1-yl)cyclobutan-1-ol (10 mg, 26.42 μmol) and 2-(2,6-dioxo-3-piperidyl)-5-fluoro-isoindoline-1,3-dione (7.30 mg, 26.42 μmol) in DMSO (1.5 mL) was added DIPEA (6.83 mg, 52.84 μmol, 8.73 μL). The reaction mixture was stirred at 145 [symbol] for 1 h under N₂, before it was purified by prep-HPLC to afford the title compound (2.9 mg, yield: 17%) as a yellow solid. MS (ESI) m/z=635.5 [M+H]⁺.

### Example 122. 5-(((Trans-3-(3-cyclopropyl-4-(7-(3-hydroxycyclobutyl)quinoxalin-2-yl)-1H-pyrazol-1-yl)cyclobutyl)methyl)amino)-2-(2,6-dioxopiperidin-3-yl)isoindoline-1,3-dione (GS-764)

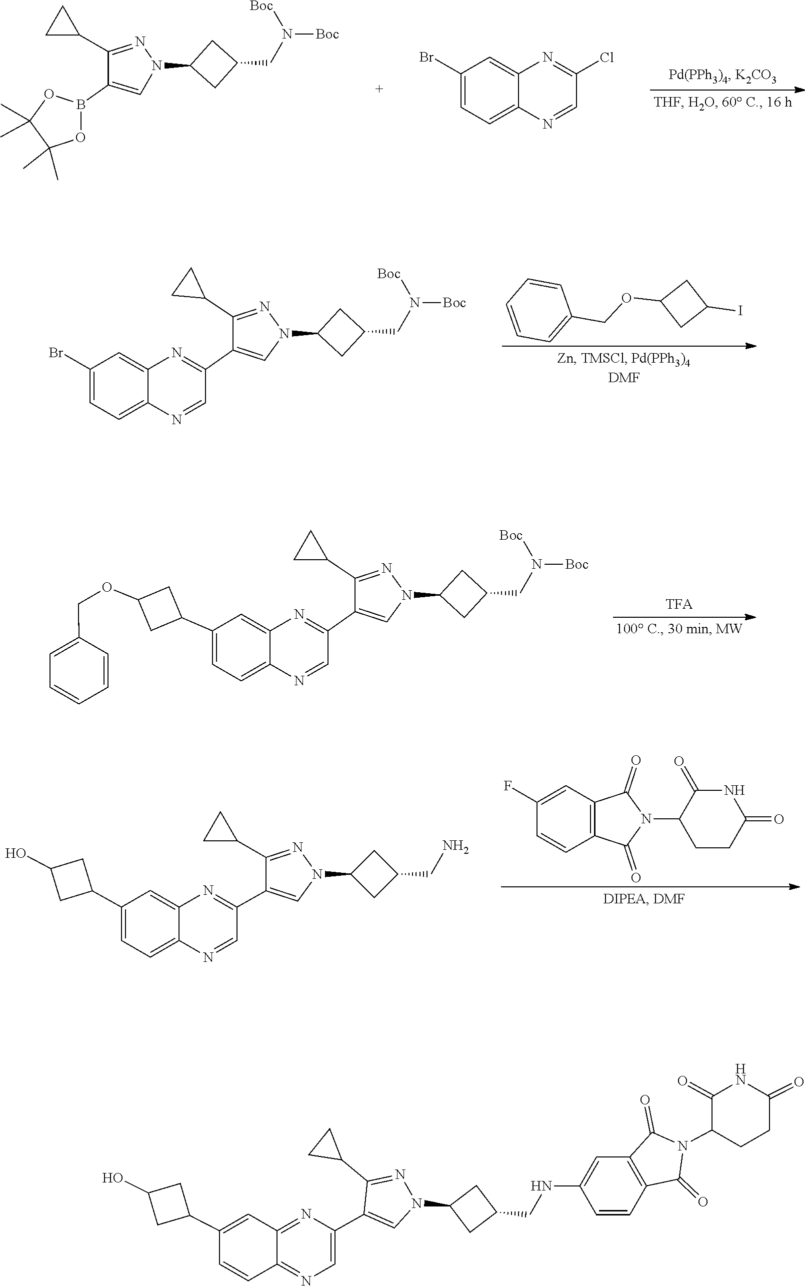

### Step 1. Synthesis of tert-butyl ((trans-3-(4-(7-bromoquinoxalin-2-yl)-3-cyclopropyl-1H-pyrazol-1-yl)cyclobutyl)methyl)(tert-butoxycarbonyl)carbamate

To a solution of tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (795.44 mg, 1.54 mmol) in THF (30 mL) were added 7-bromo-2-chloro-quinoxaline (393 mg, 1.61 mmol), tetrakis(triphenylphosphine)palladium (177.54 mg, 153.72 μmol), potassium carbonate (212.45 mg, 1.54 mmol) and water (3 mL). The mixture was stirred at 60 [symbol] for 16 h, before it was concentrated. The residue was purified by silica gel chromatography (petroleum ether:EtOAc=5:1) to afford the title compound (320 mg, yield: 35%) as a yellow oil. MS (ESI) m/z=598.1 [M+H]⁺.

### Step 2. Synthesis of tert-butyl ((trans-3-(4-(7-(3-(benzyloxy)cyclobutyl)quinoxalin-2-yl)-3-cyclopropyl-1H-pyrazol-1-yl)cyclobutyl)methyl)(tert-butoxycarbonyl)carbamate

To a suspension of Zn (27.15 mg, 417.69 μmol) in DMF (5 mL) was added TMSCl (0.9 mg, 8.35 μmol). The mixture was stirred at 70 [symbol] for 10 min. After addition of 1,2-dibromoethane (1.57 mg, 8.35 μmol), the mixture was stirred at 40 [symbol] for 10 min. (3-Iodocyclobutoxy)methylbenzene (24.07 mg, 83.54 μmol) was added. After stirring at 50 [symbol] for 20 min, tert-butyl ((trans-3-(4-(7-bromoquinoxalin-2-yl)-3-cyclopropyl-1H-pyrazol-1-yl)cyclobutyl)methyl)(tert-butoxycarbonyl)carbamate (50 mg, 83.54 μmol) and Pd(PPh₃)₄ (9.65 mg, 8.35 μmol) were added. The resulting mixture was stirred at 60 [symbol] for 2 h, before it was quenched with aq. NaHCO₃ solution. The mixture was filtered and extracted with EtOAc (20 mL). The organic phase was concentrated, and the residue was purified by silica gel chromatography (petroleum ether:EtOAc=10:1) to afford the title compound (46 mg, yield: 81%) as a yellow oil. MS (ESI) m/z=680.4 [M+H]⁺.

### Step 3. Synthesis of 3-(3-(1-(trans-3-(aminomethyl)cyclobutyl)-3-cyclopropyl-1H-pyrazol-4-yl)quinoxalin-6-yl)cyclobutan-1-ol

A solution of tert-butyl ((trans-3-(4-(7-(3-(benzyloxy)cyclobutyl)quinoxalin-2-yl)-3-cyclopropyl-1H-pyrazol-1-yl)cyclobutyl)methyl)(tert-butoxycarbonyl)carbamate (46 mg, 67.66 μmol) in TFA (1 mL) was stirred at 100 [symbol] for 30 min under microwave irradiation. The mixture was concentrated. To the resulting residue were added THF (1 mL) and NaOH solution (1 M, 0.5 mL). After stirring at rt for 1 h, the reaction mixture was purified by reverse phase chromatography (0.1% TFA in water:MeOH=1:1) to afford the title compound (20 mg, yield: 76%) as a yellow oil. MS (ESI) m/z=390.3 [M+H]⁺.

### Step 4. Synthesis of 5-(((trans-3-(3-cyclopropyl-4-(7-(3-hydroxycyclobutyl)quinoxalin-2-yl)-1H-pyrazol-1-yl)cyclobutyl)methyl)amino)-2-(2,6-dioxopiperidin-3-yl)isoindoline-1,3-dione

To a solution of 3-(3-(1-(trans-3-(aminomethyl)cyclobutyl)-3-cyclopropyl-1H-pyrazol-4-yl)quinoxalin-6-yl)cyclobutan-1-ol (10 mg, 25.67 μmol) in DMSO (2 mL) were added 2-(2,6-dioxo-3-piperidyl)-5-fluoro-isoindoline-1,3-dione (7.09 mg, 25.67 μmol), and DIPEA (3.32 mg, 25.67 μmol, 4.24 μL). The mixture was stirred at 145 [symbol] for 4 h, before it was purified by reverse phase chromatography (0.1% TFA in water:MeOH=1:1) to afford the title compound (7 mg, yield: 42%) as a yellow solid. MS (ESI) m/z=646.3 [M+H]⁺.

### Example 123. 5-(((Trans-3-(3-cyclopropyl-7-(piperazin-1-yl)-1H-indazol-1-yl)cyclobutyl)methyl)amino)-2-(2,6-dioxopiperidin-3-yl)isoindoline-1,3-dione (GS-765)

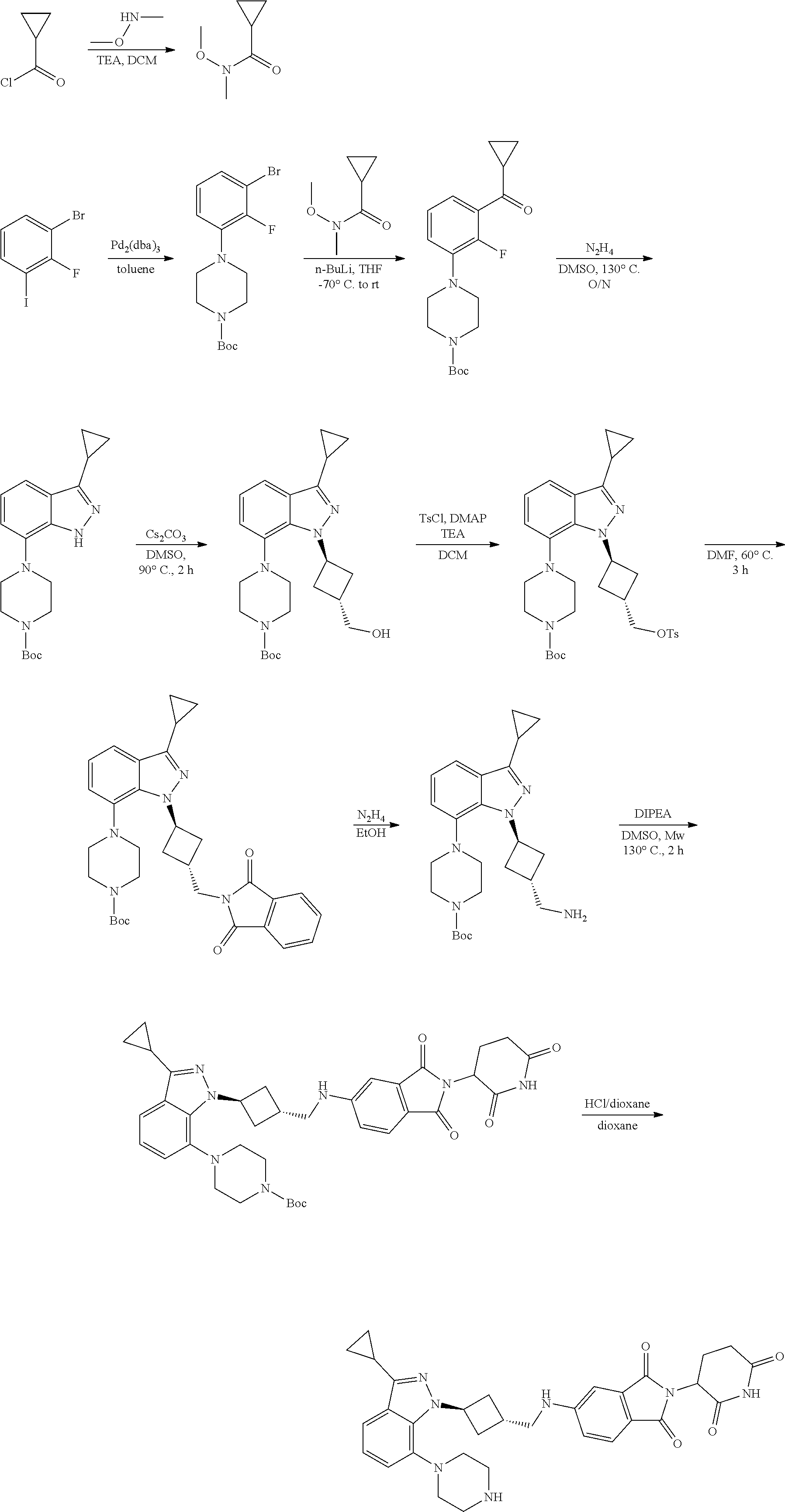

### Step 1. Synthesis of N-methoxy-N-methylcyclopropanecarboxamide

To a mixture of cyclopropanecarbonyl chloride (5 g, 47.8 mmol) and TEA (4.8 g, 47.8 mmol) in DCM (80 mL) was added N-methoxymethanamine hydrochloride (5.6 g, 57.4 mmol) at 0° C. The reaction mixture was stirred at rt for 1 h, before it was quenched with aqueous NaHCO₃ solution. The mixture was extracted with EtOAc. The organic layer was washed with water and brine, dried over Na₂SO₄, and filtered. The filtration was concentrated to afford the title compound (5 g, yield: 81%) as a bright oil.

### Step 2. Synthesis of tert-butyl 4-(3-bromo-2-fluorophenyl)piperazine-1-carboxylate

To a mixture of 1-bromo-2-fluoro-3-iodo-benzene (3.9 g, 12.9 mmol) and tert-butyl piperazine-1-carboxylate (2.5 g, 13.6 mmol) in toluene (10 mL) were added Pd₂(dba)₃ (1.2 g, 1.3 mmol), BINAP (1.6 g, 2.6 mmol) and t-BuONa (3.7 g, 38.9 mmol). After the reaction mixture was stirred at 90 [symbol] overnight, it was quenched with water, and extracted with EtOAc. The organic layer was washed with water and brine, dried over Na₂SO₄, and filtered. The filtration was concentrated. The residue was purified by silica gel chromatography (petroleum ether:EtOAc=10:0 to 10:1) to afford the title compound (1.7 g, yield: 37%) as a white solid.

### Step 3. Synthesis of tert-butyl 4-(3-(cyclopropanecarbonyl)-2-fluorophenyl)piperazine-1-carboxylate

To a solution of tert-butyl 4-(3-bromo-2-fluorophenyl)piperazine-1-carboxylate (1.7 g, 4.7 mmol) in THF (5 mL) was added n-BuLi (363 mg, 5.7 mmol) at −70 [symbol] under N₂. The reaction mixture was stirred at this temperature for 20 min, before N-methoxy-N-methylcyclopropanecarboxamide (917 mg, 7.1 mmol) was added. After the resulting mixture was stirred at rt for 3 h, it was quenched with aqueous NH₄Cl solution, and extracted with EtOAc. The organic layer was washed with water and brine, dried over Na₂SO₄, and filtered. The filtration was concentrated. The residue was purified by silica gel chromatography (petroleum ether:EtOAc=10:0 to 2:1) to afford the title compound (530 mg, yield: 32%) as a bright oil.

### Step 4. Synthesis of tert-butyl 4-(3-cyclopropyl-1H-indazol-7-yl)piperazine-1-carboxylate

To a solution of tert-butyl 4-(3-(cyclopropanecarbonyl)-2-fluorophenyl)piperazine-1-carboxylate (430 mg, 1.2 mmol) in DMSO (2 mL) was added hydrazine (790 mg, 24.7 mmol). The reaction mixture was stirred at 130 [symbol] overnight, before it was purified by reverse phase chromatography (ACN in 0.05% TFA, 0-70%) to afford the title compound (190 mg, yield: 45%) as a white solid. MS (ESI) m/z=343.3 [M+H]⁺.

### Step 5. Synthesis of tert-butyl 4-(3-cyclopropyl-1-(trans-3-(hydroxymethyl)cyclobutyl)-1H-indazol-7-yl)piperazine-1-carboxylate

To a mixture of tert-butyl 4-(3-cyclopropyl-1H-indazol-7-yl)piperazine-1-carboxylate (190 mg, 554.8 μmol) and cis-3-(hydroxymethyl)cyclobutyl 4-methylbenzenesulfonate (213 mg, 832.3 μmol) in DMSO (5 mL) was added Cs₂CO₃ (362 mg, 1.1 mmol). After the reaction mixture was stirred at 90 [symbol] for 4 h, it was quenched with water, and extracted with EtOAc. The organic layer was washed with water and brine, dried over Na₂SO₄, and filtered. The filtration was concentrated. The residue was purified by reverse phase chromatography (ACN in 0.05% TFA, 0-70%) to afford the title compound (70 mg, yield: 30%) as a white solid. MS (ESI) m/z=427.3 [M+H]⁺.

### Step 6 to 10. Synthesis of 5-(((trans-3-(3-cyclopropyl-7-(piperazin-1-yl)-1H-indazol-1-yl)cyclobutyl)methyl)amino)-2-(2.6-dioxopiperidin-3-yl)isoindoline-1,3-dione

GS-765 was synthesized following the procedures for steps 3 to 7 of GS-742 (8.1 mg, yield: 12% over 5 steps) as a yellow solid. MS (ESI) m/z=582.7 [M+H]⁺.

### Example 124. 3-(5-(2-(Trans-3-(3-cyclopropyl-4-(1-methyl-1H-pyrazolo[4,3-c]pyridin-6-yl)-1H-pyrazol-1-yl)cyclobutyl)ethyl)-1-oxoisoindolin-2-yl)piperidine-2,6-dione (GS-766)

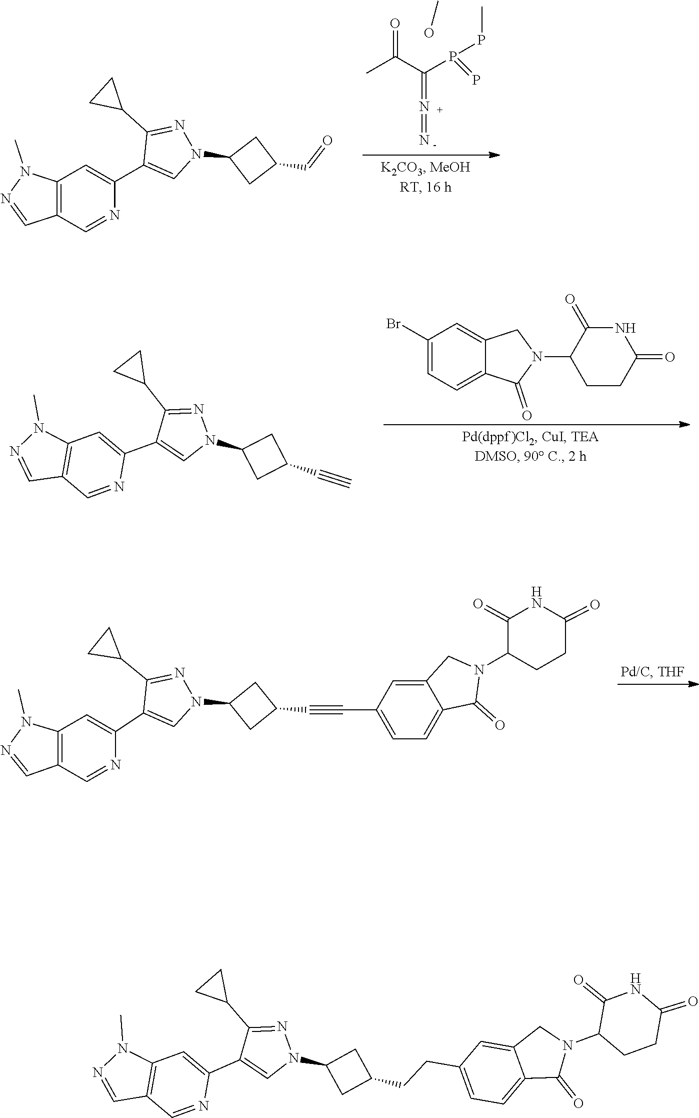

### Step 1. Synthesis of 6-(3-cyclopropyl-1-(trans-3-ethynylcyclobutyl)-1H-pyrazol-4-yl)-1-methyl-1H-pyrazolo[4,3-c]pyridine

To a solution of trans-3-(3-cyclopropyl-4-(1-methyl-1H-pyrazolo[4,3-c]pyridin-6-yl)-1H-pyrazol-1-yl)cyclobutane-1-carbaldehyde (500 mg, 1.56 mmol) and 1-diazo-1-dimethoxyphosphoryl-propan-2-one (298.89 mg, 1.56 mmol) in MeOH (10 mL) was added K₂CO₃ (1.08 g, 7.78 mmol). The mixture was stirred at 25 [symbol] for 3 h, before it was quenched with sat. NH₄Cl solution. The mixture was extracted with DCM (15 mL×3). The combined organic phase was washed with brine (50 mL), dried over anhydrous Na₂SO₄ and concentrated in vacuo. The residue was purified by silica gel chromatography (petroleum ether:EtOAc=2:3) to afford the title compound (170 mg, yield: 34%) as a colorless oil.

### Step 2. Synthesis of 3-(5-((trans-3-(3-cyclopropyl-4-(1-methyl-1H-pyrazolo[4,3-c]pyridin-6-yl)-1H-pyrazol-1-yl)cyclobutyl)ethynyl)-1-oxoisoindolin-2-yl)piperidine-2,6-dione

To a solution of 6-(3-cyclopropyl-1-(trans-3-ethynylcyclobutyl)-1H-pyrazol-4-yl)-1-methyl-1H-pyrazolo[4,3-c]pyridine (80.0 mg, 252.06 μmol) and 3-(5-bromo-1-oxo-isoindolin-2-yl)piperidine-2,6-dione (81.45 mg, 252.06 μmol) in DMSO (5 mL) were added Pd(dppf)Cl₂ (18.43 mg, 25.21 μmol), CuI (4.80 mg, 25.21 μmol) and TEA (127.53 mg, 1.26 mmol). After stirring for 2 h at 80 [symbol] under N₂, the mixture was extracted and concentrated under reduced pressure. The residue was purified by silica gel chromatography (DCM:MeOH=30:1) to afford the title compound (31.2 mg, yield: 21%) as a brown solid. MS (ESI) m/z=560.7 [M+H]⁺.

### Step 3. Synthesis of 3-(5-(2-(trans-3-(3-cyclopropyl-4-(1-methyl-1H-pyrazolo[4,3-c]pyridin-6-yl)-1-pyrazol-1-yl)cyclobutyl)ethyl)-1-oxoisoindolin-2-yl)piperidine-2,6-dione

To a solution of 3-(5-((trans-3-(3-cyclopropyl-4-(1-methyl-1H-pyrazolo[4,3-c]pyridin-6-yl)-1H-pyrazol-1-yl)cyclobutyl)ethynyl)-1-oxoisoindolin-2-yl)piperidine-2,6-dione (31.2 mg, 53.61 μmol) in THF (5 mL) was added Pd/C (19.53 mg, 160.82 μmol) under N₂. The mixture was stirred at 25 [symbol] for 1 h under balloon pressure of H₂. The resulting mixture was filtered through Celite. The filtrate was concentrated under reduced pressure. The residue was purified by prep-TLC (DCM:MeOH=10:1) to afford the title compound (2.5 mg, yield: 8%) as a white solid. MS (ESI) m/z=564.7 [M+H]⁺.

### Example 125. 5-(2-(Trans-3-(3-cyclopropyl-4-(1-methyl-1H-pyrazolo[4,3-c]pyridin-6-yl)-1H-pyrazol-1-yl)cyclobutyl)ethyl)-2-(2,6-dioxopiperidin-3-yl)isoindoline-1,3-dione (GS-767)

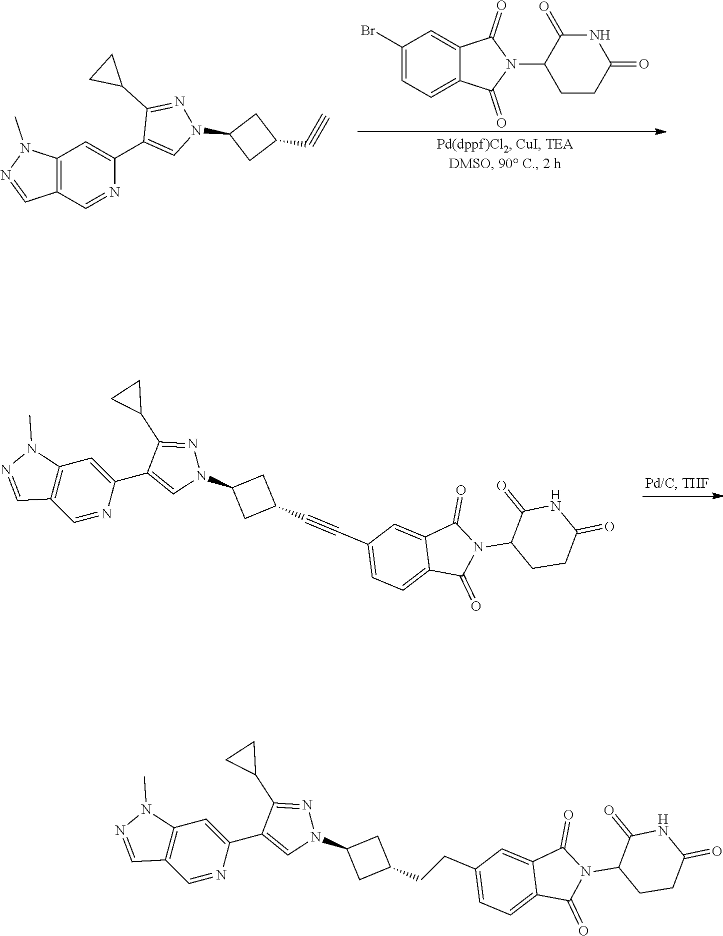 [SMILES: C#C[C@H]1C[C@H](n2cc(-c3cc4c(cn3)cnn4C)c(C3CC3)n2)C1 ; O=C1CCC(N2C(=O)c3ccc(Br)cc3C2=O)C(=O)N1 ; Cn1ncc2cnc(-c3cn([C@H]4C[C@H](C#Cc5ccc6c(c5)C(=O)N(C5CCC(=O)NC5=O)C6=O)C4)nc3C3CC3)cc21 ; Cn1ncc2cnc(-c3cn([C@H]4C[C@H](CCc5ccc6c(c5)C(=O)N(C5CCC(=O)NC5=O)C6=O)C4)nc3C3CC3)cc21]

GS-767 was synthesized following the procedures for steps 2 to 3 of GS-766 (2.6 mg, yield: 2% over 2 steps) as a white solid. MS (ESI) m/z=578.7 [M+H]⁺.

### Example 126. 5-(((Trans-3-(3-cyclopropyl-4-(7-fluoro-1-methyl-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-768)

### Step 1. Synthesis of 4-chloro-7-fluoro-1-methyl-1H-pyrrolo[3,2-c]pyridine

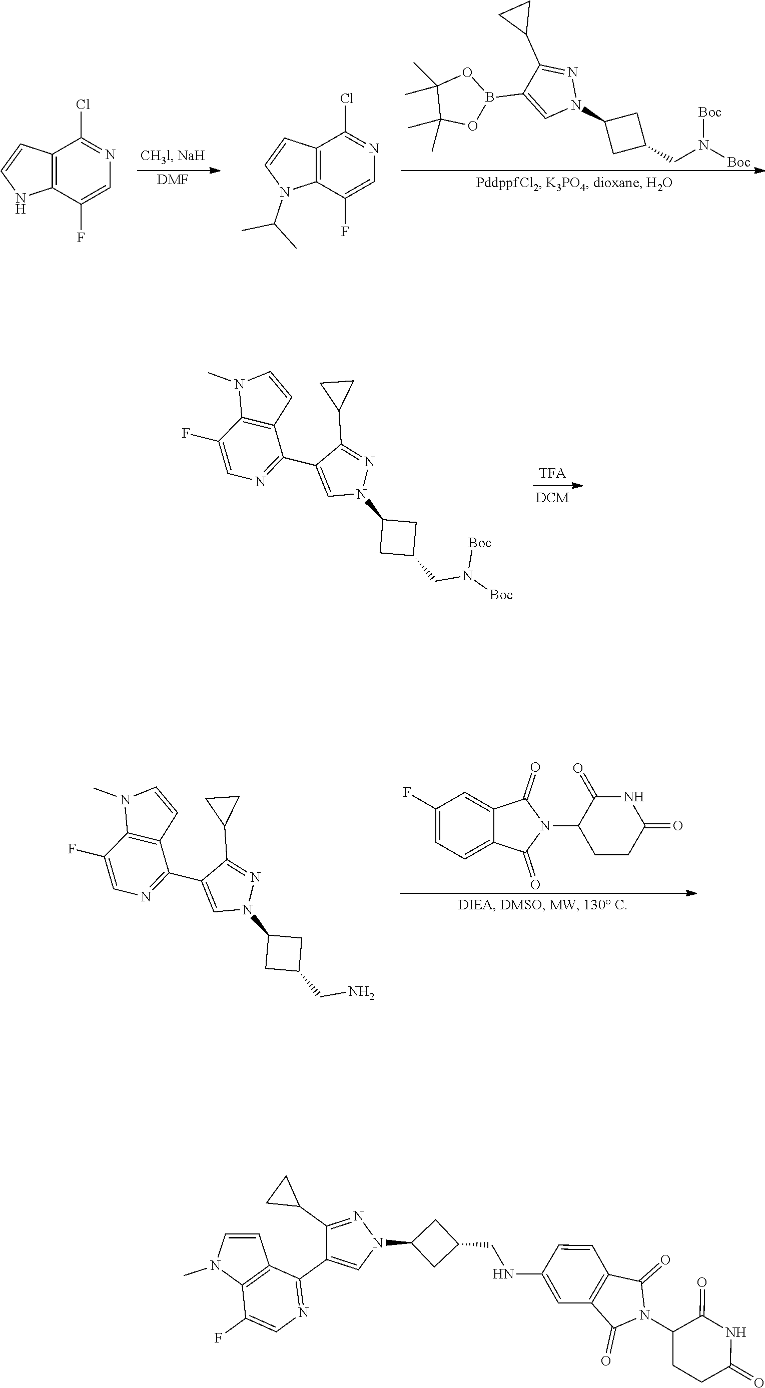

To a solution of 4-chloro-7-fluoro-1H-pyrrolo[3,2-c]pyridine (1.0 g, 5.88 mmol) in DMF (10 mL) was added NaH (705 mg, 17.64 mmol) at 0 [symbol] . The mixture was stirred at 0 [symbol] for 0.5 h, before CH₃I (1.25 g, 8.82 mmol) was added dropwise. After stirring for 2 h, the reaction mixture was diluted with ethyl acetate (20 mL) and washed with H₂O and brine. The organic layer was concentrated, and the residue was purified by silica gel chromatography to afford the title compound (750 mg, yield: 69%) as a colorless oil. MS (ESI) m/z=185.6 [M+H]⁺.

### Step 2. Synthesis of tert-butyl (tert-butoxycarbonyl)((trans-3-(3-cyclopropyl-4-(7-fluoro-1-methyl-1H-pyrrolo[3,2-c]pyridin-4-yl)-1H-pyrazol-1-yl)cyclobutyl)methyl)carbamate

To a solution of 4-chloro-7-fluoro-1-methyl-1H-pyrrolo[3,2-c]pyridine (750 mg, 4.03 mmol) and di-tert-butyl ((trans-3-(3-cyclopropyl-4-(4,4,5,5-tetramethyl-1,3,2-dioxaborolan-2-yl)-1H-pyrazol-1-yl)cyclobutyl)methyl)carbamate (3.12 g, 6.04 mmol) in 1,4-dixoane (30 mL) and H₂O (6 mL) were added Pd(dppf)Cl₂ (652 mg, 0.8 mmol) and K₂CO₃ (1.66 g, 12.09 mmol). After stirring for 3 h at 90 [symbol] under N₂, the mixture was concentrated under reduced pressure. The residue was purified by silica gel chromatography to afford the title compound (493 mg, yield: 23%) as a colorless oil. MS (ESI) m/z=540.65 [M+H]⁺.

### Step 3. Synthesis of (trans-3-(3-cyclopropyl-4-(7-fluoro-1-methyl-1H-pyrrolo[3,2-c]pyridin-4-yl)-1H-pyrazol-1-yl)cyclobutyl)methanamine

To a solution of tert-butyl (tert-butoxycarbonyl)((trans-3-(3-cyclopropyl-4-(7-fluoro-1-methyl-1H-pyrrolo[3,2-c]pyridin-4-yl)-1H-pyrazol-1-yl)cyclobutyl)methyl)carbamate (493 mg, 0.91 mmol) in DCM (4 mL) was added TFA (2 mL). The reaction mixture was stirred at rt for 1 h, before it was concentrated. The residue was purified by reverse phase chromatography (ACN in 0.05% TFA) to afford the title compound (240 mg, yield: 78%) as a yellow solid. MS (ESI) m/z=340.1 [M+H]⁺.

### Step 4. Synthesis of 5-(((trans-3-(3-cyclopropyl-4-(7-fluoro-1-methyl-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(7-fluoro-1-methyl-1H-pyrrolo[3,2-c]pyridin-4-yl)-1H-pyrazol-1-yl)cyclobutyl)methanamine (47.6 mg, 0.14 mmol) and 2-(2,6-dioxopiperidin-3-yl)-5-fluoroisoindoline-1,3-dione (45 mg, 0.16 mmol) in DMSO (2 mL) was added KF (24 mg, 0.42 mmol). The reaction mixture was stirred at 140° C. under microwave irradiation for 1 h, before it was purified by reverse phase chromatography (ACN in 0.05% TFA solution) to afford the title compound (16 mg, yield: 21%) as a yellow solid. MS (ESI) m/z=549.5 [M+H]⁺.

### Example 127. 5-(((Trans-3-(3-cyclopropyl-4-(7-fluoro-1-isopropyl-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-769)

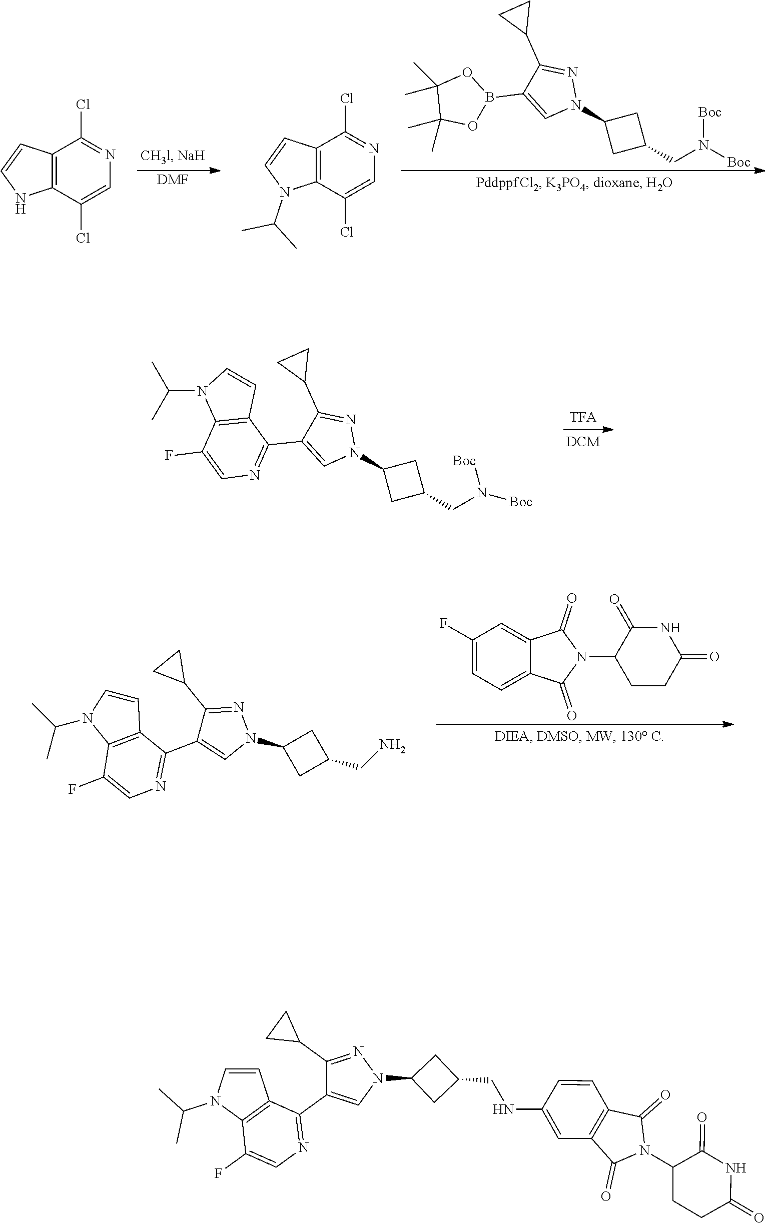

### Step 1. Synthesis of 4-chloro-7-fluoro-1-isopropyl-1H-pyrrolo[3,2-c]pyridine

To a solution of 4-chloro-7-fluoro-1H-pyrrolo[3,2-c]pyridine (1.0 g, 5.88 mmol) in DMF (10 mL) was added NaH (705 mg, 17.64 mmol) at 0 [symbol] . The mixture was stirred at this temperature for 0.5 h. 2-Bromopropane (1.07 g, 8.82 mmol) was added dropwise. After stirring for 2 h, the reaction mixture was diluted with ethyl acetate (20 mL) and washed with H₂O and brine. The organic layer was concentrated and the residue was purified by silica gel chromatography to afford the title compound (450 mg, yield: 36%) as a colorless oil. MS (ESI) m/z=213.65 [M+H]⁺.

### Step 2. Synthesis of tert-butyl (tert-butoxycarbonyl)((trans-3-(3-cyclopropyl-4-(7-fluoro-1-isopropyl-1H-pyrrolo[3,2-c]pyridin-4-yl)-1H-pyrazol-1-yl)cyclobutyl)methyl)carbamate

To a solution of 4-chloro-7-fluoro-1-isopropyl-1H-pyrrolo[3,2-c]pyridine (450 mg, 2.11 mmol) and di-tert-butyl ((trans-3-(3-cyclopropyl-4-(4,4,5,5-tetramethyl-1,3,2-dioxaborolan-2-yl)-1H-pyrazol-1-yl)cyclobutyl)methyl)carbamate (1.64 g, 3.17 mmol) in 1,4-dioxane (30 mL) and H₂O (6 mL) were added Pd(dppf)Cl₂ (170 mg, 0.21 mmol) and K₂CO₃ (869 mg, 6.3 mmol). After stirring for 3 h at 90 [symbol] under N₂, the reaction mixture was concentrated under reduced pressure. The residue was purified by silica gel chromatography to give the desired product (200 mg, yield: 16%) as a colorless oil. MS (ESI) m/z=568.71 [M+H]⁺.

### Step 3. Synthesis of (trans-3-(3-cyclopropyl-4-(7-fluoro-1-isopropyl-1H-pyrrolo[3,2-c]pyridin-4-yl)-1H-pyrazol-1-yl)cyclobutyl)methanamine

To a solution of tert-butyl (tert-butoxycarbonyl)((trans-3-(3-cyclopropyl-4-(7-fluoro-1-isopropyl-1H-pyrrolo[3,2-c]pyridin-4-yl)-1H-pyrazol-1-yl)cyclobutyl)methyl)carbamate (200 mg, 0.34 mmol) in DCM (4 mL) was added TFA (2 mL). The reaction mixture was stirred at rt for 1 h, before it was concentrated. The residue was purified by reverse phase chromatography to afford the title compound (78 mg, yield: 62%) as a yellow solid. MS (ESI) m/z=368.47 [M+H]⁺.

### Step 4. Synthesis of 5-(((trans-3-(3-cyclopropyl-4-(7-fluoro-1-isopropyl-1H-pyrrolo[3,2-c]pyridin-4-yl)-1-pyrazol-1-yl)cyclobutyl)methyl)amino)-2-(2,6-dioxopiperidin-3-yl)isoindoline-1,3-dione

To a solution of (trans-3-(3-cyclopropyl-4-(7-fluoro-1-isopropyl-1H-pyrrolo[3,2-c]pyridin-4-yl)-1H-pyrazol-1-yl)cyclobutyl)methanamine (60 mg, 0.16 mmol) and 2-(2,6-dioxopiperidin-3-yl)-5-fluoroisoindoline-1,3-dione (51 mg, 0.16 mmol) in DMSO (2 mL) was added KF (92.8 mg, 1.6 mmol). The reaction mixture was stirred at 140° C. under microwave irradiation for 1 h, before it was purified by reverse phase chromatography to afford the title compound (8.0 mg, yield: 8%) as a yellow solid. MS (ESI) m/z=624.69 [M+H]⁺.

### Example 128. 5-(((Trans-3-(3-cyclopropyl-4-(1-(1-methylpiperidin-4-yl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-770)

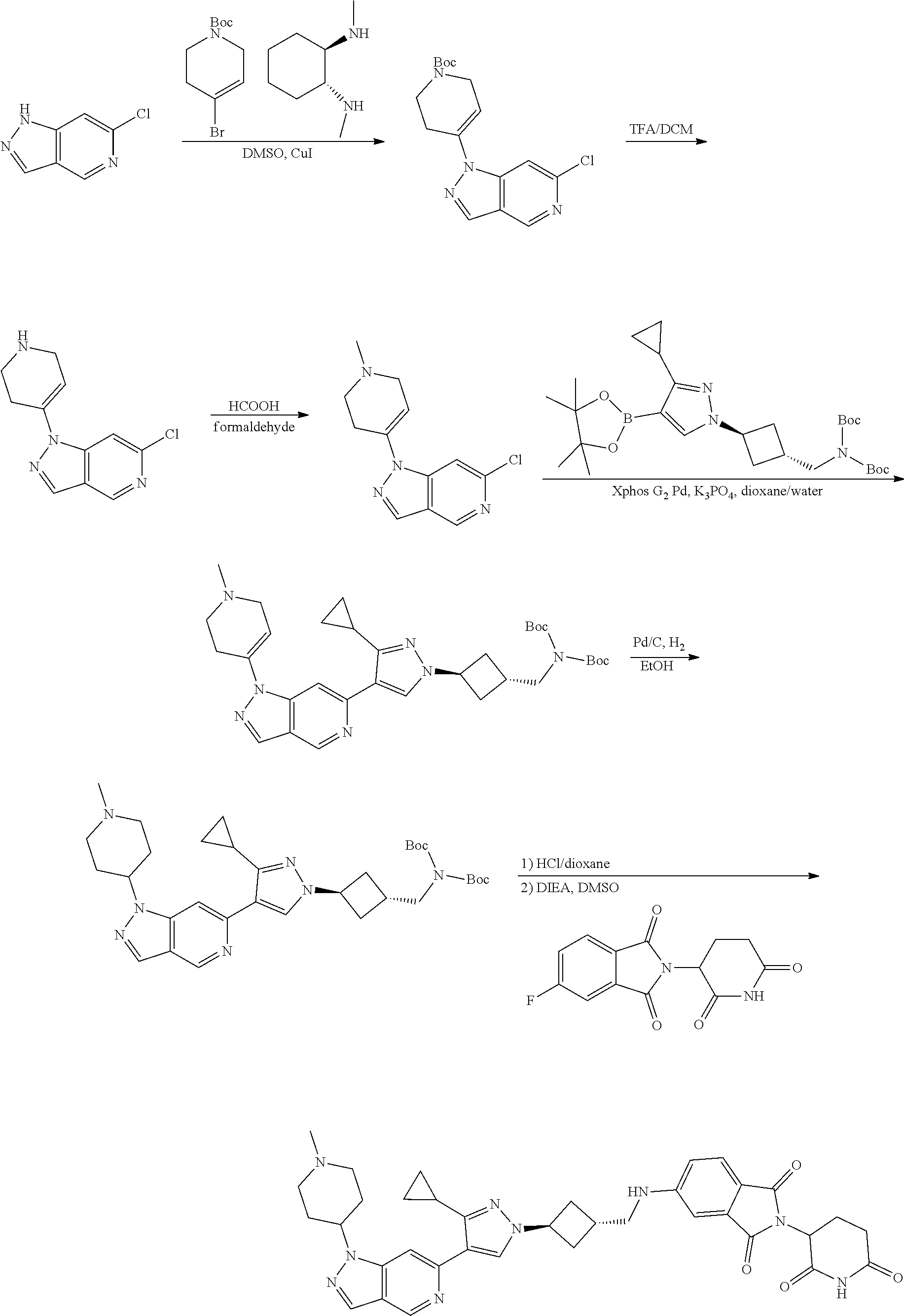

### Step 1. Synthesis of tert-butyl 4-(6-chloro-1H-pyrazolo[4,3-c]pyridin-1-yl)-3,6-dihydropyridine-1 (2H)-carboxylate

A mixture of 6-chloro-1H-pyrazolo[4,3-c]pyridine (1 g, 6.51 mmol), tert-butyl 4-bromo-3,6-dihydro-2H-pyridine-1-carboxylate (2.56 g, 9.77 mmol), trans-N1,N2-dimethylcyclohexane-1,2-diamine (1 g, 5.87 mmol), K₃PO₄ (4.15 g, 19.54 mmol) and CuI (500 mg, 2.63 mmol) in DMSO (10 mL) was heated at 100 [symbol] under N₂ overnight. After cooling to rt, the mixture was poured into water (100 mL) and filtered. The filter cake was purified by silica gel chromatography to afford the title compound (1.2 g, yield: 48%) as a brown solid. MS (ESI) m/z=335.2 [M+H]⁺.

### Step 2. Synthesis of 6-chloro-1-(1,2,3,6-tetrahydropyridin-4-yl)-1H-pyrazolo[4.3-c]pyridine

To a solution of tert-butyl 4-(6-chloro-1H-pyrazolo[4,3-c]pyridin-1-yl)-3,6-dihydropyridine-1 (2H)-carboxylate (500 mg, 1.31 mmol) in DCM (5 mL) was added TFA (2 mL) at it. The mixture was stirred for 1 h, before it was concentrated to afford the title compound (250 mg, yield: 73%) as a light brown oil. MS (ESI) m/z=235.1 [M+H]⁺.

### Step 3. Synthesis of 6-chloro-1-(1-methyl-1,2,3,6-tetrahydropyridin-4-yl)-1H-pyrazolo[4,3-c]pyridine

To a solution of 6-chloro-1-(1,2,3,6-tetrahydropyridin-4-yl)-1H-pyrazolo[4,3-c]pyridine (100 mg, 383.49 μmol) in HCOOH (8 mL) was added formaldehyde solution (37 wt. % in H₂O, 1 mL, 13 mmol) at rt. The reaction mixture was heated at 90 [symbol] overnight, before it was concentrated. The residue was purified by reverse phase chromatography to afford the title compound (50 mg, yield: 52%) as a colorless oil. MS (ESI) m/z=249.1 [M+H]⁺.

### Step 4. Synthesis of tert-butyl (tert-butoxycarbonyl)((trans-3-(3-cyclopropyl-4-(1-(1-methyl-1,2,3,6-tetrahydropyridin-4-yl)-1H-pyrazolo[4,3-c]pyridin-6-yl)-1H-pyrazol-1-yl)cyclobutyl)methyl)carbamate

A mixture of 6-chloro-1-(1-methyl-1,2,3,6-tetrahydropyridin-4-yl)-1H-pyrazolo[4,3-c]pyridine (50 mg, 199.03 μmol), tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (150 mg, 260.89 μmol), K₃PO₄ (130 mg, 612.63 μmol) and XPhos Pd G2 (25 mg, 199.03 μmol) in dioxane (6 mL) and water (2 mL) was heated at 100 [symbol] overnight under N₂. The mixture was cooled to rt and concentrated. The residue was purified by reverse phase chromatography to afford the title compound (50 mg, yield: 410%) as a light yellow oil. MS (ESI) m/z=604.4 [M+H]⁺.

### Step 5. Synthesis of tert-butyl (tert-butoxycarbonyl)((trans-3-(3-cyclopropyl-4-(1-(1-methylpiperidin-4-yl)-1H-pyrazolo[4,3-c]pyridin-6-yl)-1H-pyrazol-1-yl)cyclobutyl)methyl)carbamate

A mixture of tert-butyl (tert-butoxycarbonyl)((trans-3-(3-cyclopropyl-4-(1-(1-methyl-1,2,3,6-tetrahydropyridin-4-yl)-1H-pyrazolo[4,3-c]pyridin-6-yl)-1H-pyrazol-1-yl)cyclobutyl)methyl)carbamate (50 mg, 81.16 μmol) and Pd/C (20 mg, 164.67 μmol) in ethanol (8 mL) was stirred under H₂ at rt overnight. The mixture was filtered and concentrated to afford the title compound (40 mg, yield: 74%) as a colorless oil. MS (ESI) m/z=606.4 [M+H]⁺.

### Step 6. Synthesis of 5-(((trans-3-(3-cyclopropyl-4-(1-(1-methylpiperidin-4-yl)-1H-pyrazolo[4,3-c]pyridin-6-yl)-H-pyrazol-1-yl)cyclobutyl)methyl)amino)-2-(2,6-dioxopiperidin-3-yl)isoindoline-1,3-dione

To a solution of tert-butyl (tert-butoxycarbonyl)((trans-3-(3-cyclopropyl-4-(1-(1-methylpiperidin-4-yl)-1H-pyrazolo[4,3-c]pyridin-6-yl)-1H-pyrazol-1-yl)cyclobutyl)methyl)carbamate (25 mg, 37.56 μmol) in DCM (3 mL) was added HCl solution (4 M in dioxane, 1 mL) at rt. The reaction mixture was stirred for 1 h, before it was concentrated. The resulting residue, 2-(2,6-dioxopiperidin-3-yl)-5-fluoroisoindoline-1,3-dione (10 mg, 36.20 μmol) and DIPEA (25 mg, 193.80 μmol) were dissolved in DMSO (1 mL). The mixture was heated at 140 [symbol] under N₂ for 6 h, before it was purified by reverse phase chromatography to afford the title compound (3 mg, yield: 12%) as a yellow solid. MS (ESI) m/z=662.5 [M+H]⁺.

### Examples 129 and 130. 5-((((Trans-3-(3-cyclopropyl-4-(3-(cis-3-hydroxycyclobutyl)pyridin-2-yl)-1H-pyrazol-1-yl)cyclobutyl)methyl)amino)-2-(2,6-dioxopiperidin-3-yl)isoindoline-1,3-dione (GS-771) and 5-((((trans-3-(3-cyclopropyl-4-(3-(trans-3-hydroxycyclobutyl)pyridin-2-yl)-1H-pyrazol-1-yl)cyclobutyl)methyl)amino)-2-(2,6-dioxopiperidin-3-yl)isoindoline-1,3-dione (GS-772)

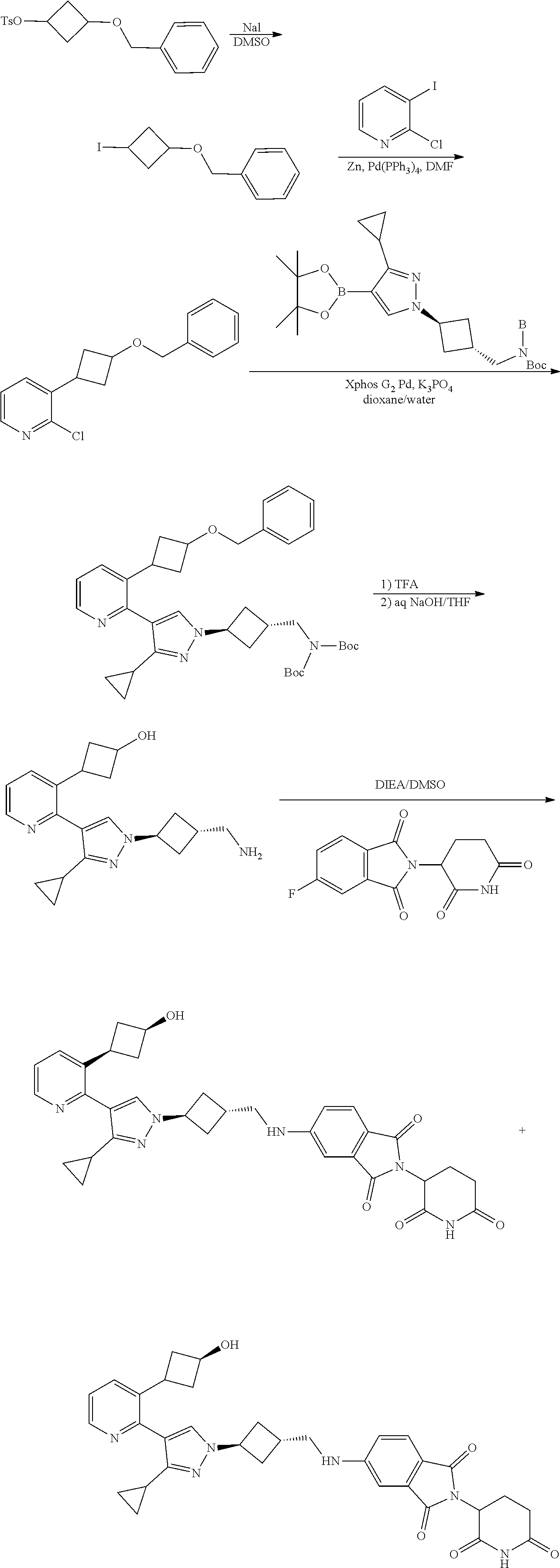

### Step 1. Synthesis of ((3-iodocyclobutoxy)methyl)benzene

A mixture of 3-(benzyloxy)cyclobutyl 4-methylbenzenesulfonate (9 g, 27.07 mmol) and NaI (20.40 g, 136.10 mmol) in DMSO (60 mL) was heated at 85 [symbol] for 5 h. The mixture was poured into water (150 mL) and extracted with EtOAc (150 mL×2). The combined organic layers were washed with water (60 mL) and brine (60 mL), dried over Na₂SO₄, and filtered. The filtration was concentrated. The residue was purified by silica gel chromatography to afford the title compound (4.5 g, yield: 58%) as a light brown oil.

### Step 2. Synthesis of 3-(3-(benzyloxy)cyclobutyl)-2-chloropyridine

To a suspension of Zn (200 mg, 3.08 mmol) in DMF (12 mL) was added TMSCl (0.1 mL) under N₂ at 70 [symbol] . The mixture was stirred at 40 [symbol] for 10 min. 1,2-Dibromoethane (0.1 mL) was added. The mixture was stirred at 40 [symbol] for 0.5 h, before ((3-iodocyclobutoxy)methyl)benzene (550 mg, 1.72 mmol) was added. The mixture was stirred at 40 [symbol] for 1 h. 2-Chloro-3-iodo-pyridine (100 mg, 417.64 μmol) and Pd(PPh₄)₃ (50 mg, 417.64 μmol) were added. The mixture was stirred at 70 [symbol] overnight. The mixture was cooled to rt and purified by reverse phase chromatography to afford the title compound (35 mg, yield: 19%) as a light yellow oil. MS (ESI) m/z=274.1 [M+H]⁺.

### Step 3. Synthesis of tert-butyl ((trans-3-(4-(3-(3-(benzyloxy)cyclobutyl)pyridin-2-yl)-3-cyclopropyl-1H-pyrazol-1-yl)cyclobutyl)methyl)(tert-butoxycarbonyl)carbamate

A mixture of 3-(3-(benzyloxy)cyclobutyl)-2-chloropyridine (35 mg, 79.27 μmol), tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (57.50 mg, 100 μmol), K₃PO₄ (55 mg, 259.43 μmol), XPhos Pd G2 (20 mg, 79.27 μmol) in dioxane (5 mL) and water (1.5 mL) was heated at 100 [symbol] under N₂ overnight. The mixture was cooled to rt, before it was concentrated. The residue was purified by reverse phase chromatography to afford the title compound (30 mg, yield: 55%) as a light brown oil. MS (ESI) m/z=629.4 [M+H]⁺.

### Step 4. Synthesis of 3-(2-(1-(trans-3-(aminomethyl)cyclobutyl)-3-cyclopropyl-1H-pyrazol-4-yl)pyridin-3-yl)cyclobutan-1-ol

A solution of tert-butyl ((trans-3-(4-(3-(3-(benzyloxy)cyclobutyl)pyridin-2-yl)-3-cyclopropyl-1H-pyrazol-1-yl)cyclobutyl)methyl)(tert-butoxycarbonyl)carbamate (30 mg, 43.42 μmol) in TFA (3 mL) was stirred at 100 [symbol] for 0.5 h under microwave irradiation. The mixture was cooled and concentrated. The residue was dissolved in THF (5 mL) and NaOH solution (3 M, 0.5 mL) at rt was added. The mixture was stirred for 0.5 h. The organic layer was separated and purified by reverse phase chromatography to afford the title compound (5 mg, yield: 31%) as a light brown oil. MS (ESI) m/z=339.3 [M+H]⁺.

### Step 5. Synthesis of 5-((((trans-3-(3-cyclopropyl-4-(3-(cis-3-hydroxycyclobutyl)pyridin-2-yl)-1H-pyrazol-1-yl)cyclobutyl)methyl)amino)-2-(2,6-dioxopiperidin-3-yl)isoindoline-1,3-dione and 5-((((trans-3-(3-cyclopropyl-4-(3-(trans-3-hydroxycyclobutyl)pyridin-2-yl)-1H-pyrazol-1-yl)cyclobutyl)methyl)amino)-2-(2,6-dioxopiperidin-3-yl)isoindoline-1,3-dione

A mixture of 3-(2-(1-(trans-3-(aminomethyl)cyclobutyl)-3-cyclopropyl-1H-pyrazol-4-yl)pyridin-3-yl)cyclobutan-1-ol (5 mg, 13.30 μmol), 2-(2,6-dioxo-3-piperidyl)-5-fluoro-isoindoline-1,3-dione (6 mg, 21.72 μmol) and DIEA (16 mg, 124.03 μmol) in DMSO (1 mL) was heated at 140 [symbol] under N₂ for 6 h. The mixture was cooled to rt and purified with prep-HPLC to afford the title compounds.

5-((((Trans-3-(3-cyclopropyl-4-(3-(cis-3-hydroxycyclobutyl)pyridin-2-yl)-1H-pyrazol-1-yl)cyclobutyl)methyl)amino)-2-(2,6-dioxopiperidin-3-yl)isoindoline-1,3-dione (0.76 mg, yield: 10%) was obtained as a yellow solid. MS (ESI) m/z=595.7 [M+H]⁺.

5-((((Trans-3-(3-cyclopropyl-4-(3-(trans-3-hydroxycyclobutyl)pyridin-2-yl)-1H-pyrazol-1-yl)cyclobutyl)methyl)amino)-2-(2,6-dioxopiperidin-3-yl)isoindoline-1,3-dione (0.91 mg, yield: 12%) was obtained as a yellow solid. MS (ESI) m/z=595.7 [M+H]⁺.

### Example 131. 5-(((Trans-3-(3-cyclopropyl-4-((1-methyl-1H-pyrazol-4-yl)amino)-1H-pyrazol-1-yl)cyclobutyl)methyl)amino)-2-(2,6-dioxopiperidin-3-yl)isoindoline-1,3-dione (GS-773)

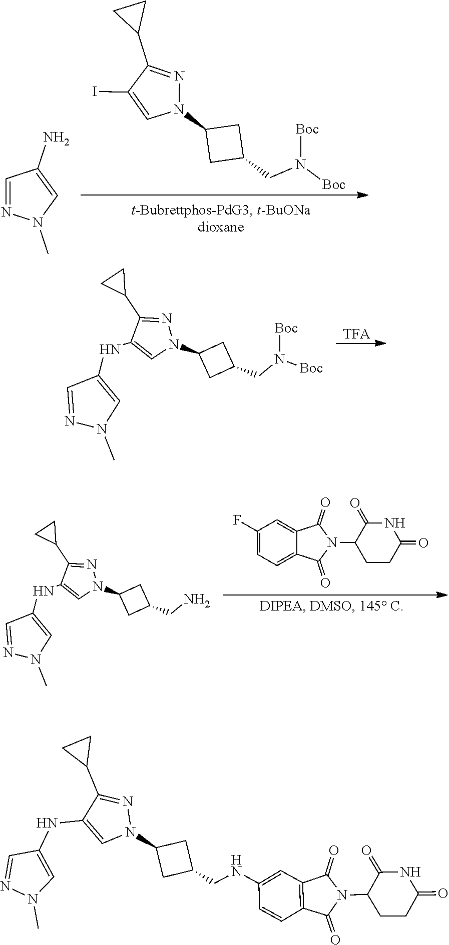

### Step 1. Synthesis of tert-butyl (tert-butoxycarbonyl)((trans-3-(3-cyclopropyl-4-((1-methyl-1H-pyrazol-4-yl)amino)-1H-pyrazol-1-yl)cyclobutyl)methyl)carbamate

To a solution of 1-methylpyrazol-4-amine (80 mg, 823.74 μmol) in dioxane (2 mL) were added tert-butyl (tert-butoxycarbonyl)((trans-3-(3-cyclopropyl-4-iodo-1H-pyrazol-1-yl)cyclobutyl)methyl)carbamate (426.20 mg, 823.74 μmol), tBuBrettPhos Pd G3 (39.29 mg, 41.19 μmol) and t-BuONa (158.16 mg, 1.65 mmol). The mixture was stirred at 90 [symbol] for 16 h, before it was purified by reverse phase chromatography (0.1% TFA in water:MeOH=1:1) to afford the title compound (20 mg, yield: 5%) as a yellow oil. MS (ESI) m/z=487.3 [M+H]⁺.

### Step 2. Synthesis of 1-(trans-3-(aminomethyl)cyclobutyl)-3-cyclopropyl-N-(1-methyl-1H-pyrazol-4-yl)-1H-pyrazol-4-amine

A solution of tert-butyl (tert-butoxycarbonyl)((trans-3-(3-cyclopropyl-4-((1-methyl-1H-pyrazol-4-yl)amino)-1H-pyrazol-1-yl)cyclobutyl)methyl)carbamate (17 mg, 34.94 μmol) in TFA (1 mL) was stirred at rt for 10 min. The mixture was purified by reverse phase chromatography (water:MeOH=1:1) to afford the title compound (9 mg, yield: 90%) as a colorless oil. MS (ESI) m/z=287.2 [M+H]⁺.

### Step 3. Synthesis of 5-(((trans-3-(3-cyclopropyl-4-((1-methyl-1H-pyrazol-4-yl)amino)-1H-pyrazol-1-yl)cyclobutyl)methyl)amino)-2-(2,6-dioxopiperidin-3-yl)isoindoline-1,3-dione

To a solution of 1-(trans-3-(aminomethyl)cyclobutyl)-3-cyclopropyl-N-(1-methyl-1H-pyrazol-4-yl)-1H-pyrazol-4-amine (10 mg, 34.92 μmol) in DMSO (3 mL) were added 2-(2,6-dioxo-3-piperidyl)-5-fluoro-isoindoline-1,3-dione (9.65 mg, 34.92 μmol) and DIPEA (9.03 mg, 69.84 μmol, 11.54 μL). The mixture was stirred at 145 [symbol] for 3 h, before it was purified by reverse phase chromatography (water:MeOH=1:1) to afford the title compound (3.6 mg, yield: 19%) as a yellow solid. MS (ESI) m/z=543.2 [M+H]⁺.

### Example 133. 6-(3-Cyclopropyl-4-(6-methylpyridin-2-yl)-1H-pyrazol-1-yl)-N-((2-(2,6-dioxopiperidin-3-yl)-1,3-dioxoisoindolin-5-yl)methyl)spiro[3.3]heptane-2-carboxamide (GS-774)

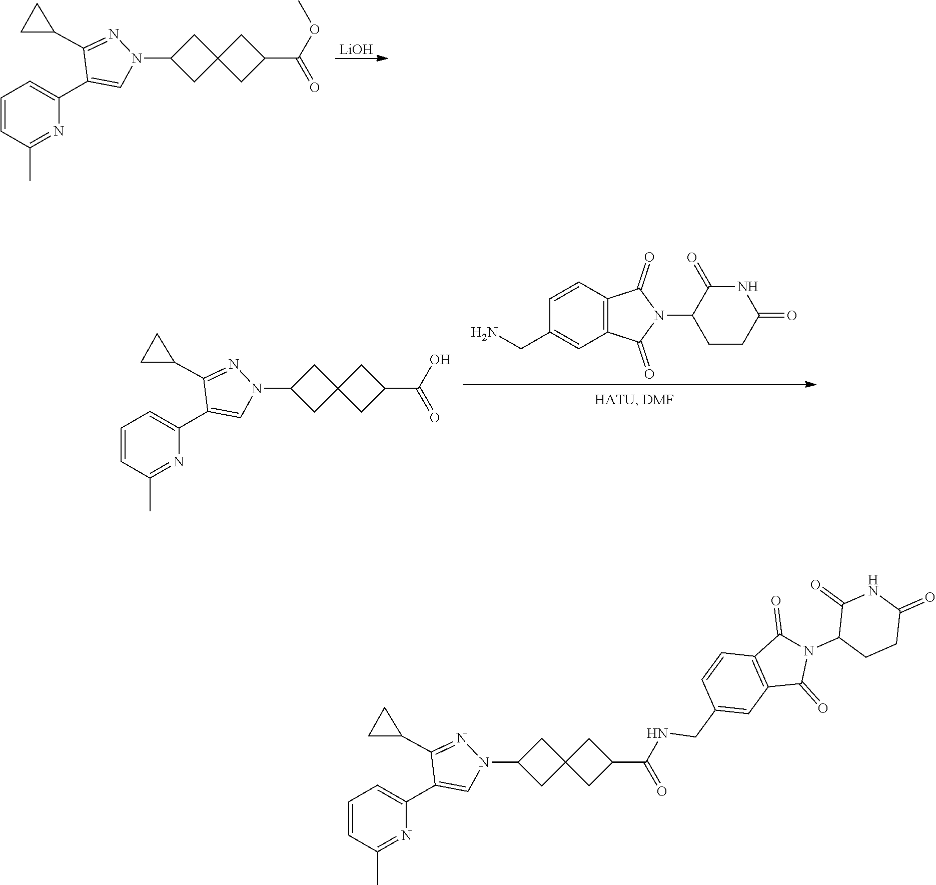

### Step 1. Synthesis of 6-(3-cyclopropyl-4-(6-methylpyridin-2-yl)-1H-pyrazol-1-yl)spiro[3.3]heptane-2-carboxylic acid

To a solution of methyl 6-(3-cyclopropyl-4-(6-methylpyridin-2-yl)-1H-pyrazol-1-yl)spiro[3.3]heptane-2-carboxylate (50 mg, 142.27 μmol) in THF (2 mL) were added LiOHH₂O (11.95 mg, 284.54 μmol) and water (1 mL). The mixture was stirred at rt for 20 min, before it was purified by reverse phase chromatography (water:MeOH=1:1) to afford the title compound (39 mg, yield: 81%) as a yellow oil. MS (ESI) m/z=338.2 [M+H]⁺.

### Step 2. Synthesis of 6-(3-cyclopropyl-4-(6-methylpyridin-2-yl)-1H-pyrazol-1-yl)-N-((2-(2,6-dioxopiperidin-3-yl)-1,3-dioxoisoindolin-5-yl)methyl)spiro[3.3]heptane-2-carboxamide

To a solution of 6-(3-cyclopropyl-4-(6-methylpyridin-2-yl)-1H-pyrazol-1-yl)spiro[3.3]heptane-2-carboxylic acid (5 mg, 14.82 μmol) in DMF (1 mL) were added 5-(aminomethyl)-2-(2,6-dioxo-3-piperidyl)isoindoline-1,3-dione (28.38 mg, 14.82 μmol) and HATU (6.76 mg, 17.78 μmol). The mixture was stirred at rt for 1 h. The residue was purified by reverse phase chromatography (water:MeOH=1:1) to afford the title compound (0.9 mg, yield: 10%) as a yellowish solid. MS (ESI) m/z=607.3 [M+H]⁺.

### Example 133. 6-(3-Cyclopropyl-4-(6-methylpyridin-2-yl)-1H-pyrazol-1-yl)-N-((2-(2,6-dioxopiperidin-3-yl)-1,3-dioxoisoindolin-5-yl)methyl)spiro[3.3]heptane-2-carboxamide (GS-775)

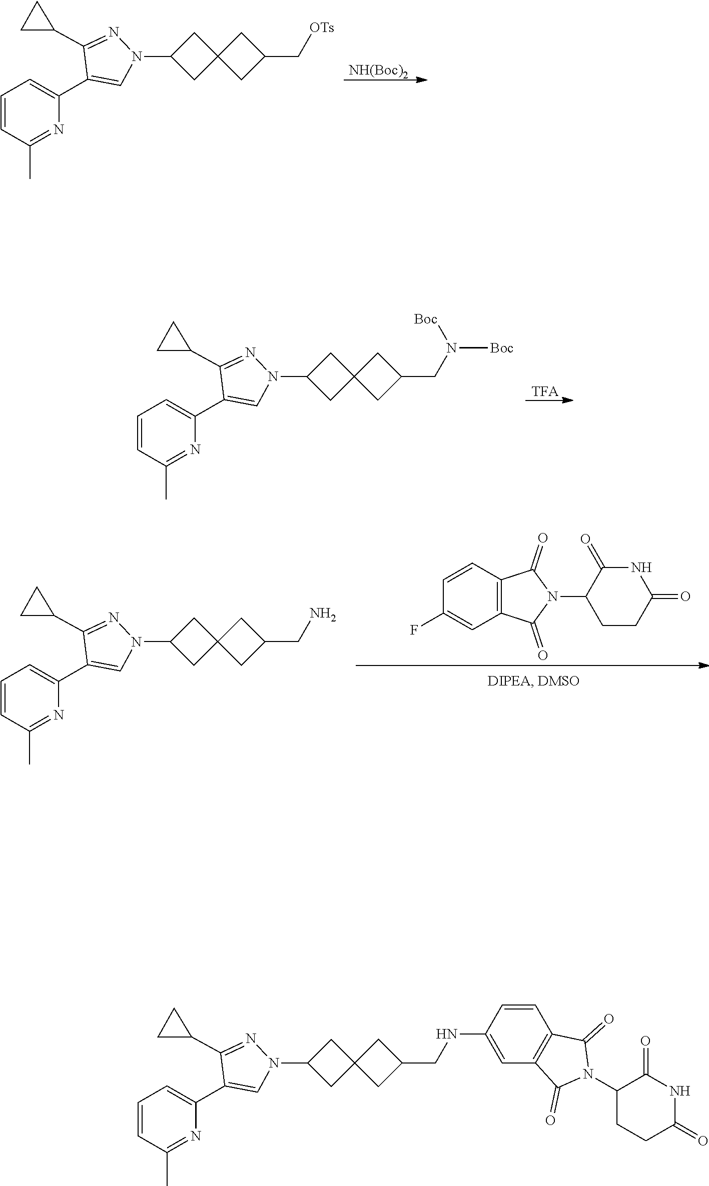 [SMILES: Cc1ccc(S(=O)(=O)OCC2CC3(C2)CC(n2cc(-c4cccc(C)n4)c(C4CC4)n2)C3)cc1 ; Cc1cccc(-c2cn(C3CC4(CC(CN(C(=O)OC(C)(C)C)C(=O)OC(C)(C)C)C4)C3)nc2C2CC2)n1 ; "NH(Boc)2" ; Cc1cccc(-c2cn(C3CC4(CC(CN)C4)C3)nc2C2CC2)n1 ; O=C1CCC(N2C(=O)c3ccc(F)cc3C2=O)C(=O)N1 ; Cc1cccc(-c2cn(C3CC4(CC(CNc5ccc6c(c5)C(=O)N(C5CCC(=O)NC5=O)C6=O)C4)C3)nc2C2CC2)n1]

GS-775 was synthesized following the procedures for steps 5 to 7 of GS-751 (4.6 mg, yield: 18% over 3 steps) as a yellow solid. MS (ESI) m/z=579.3 [M+H]⁺.

### Example 134. 5-(((Trans-3-(4-(1-(trans-3-aminocyclobutyl)-1H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione (GS-776)

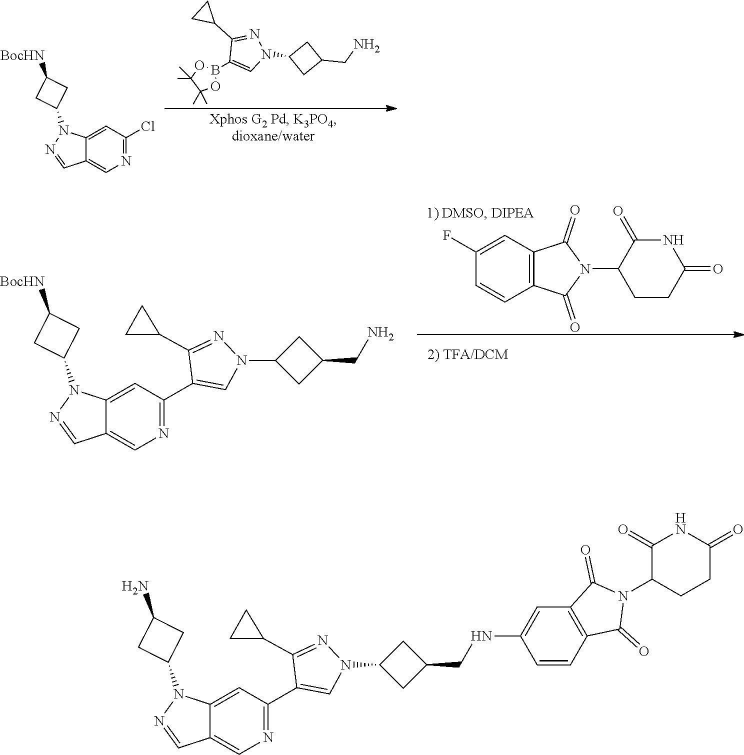

### Step 1. Synthesis of tert-butyl (trans-3-(6-(1-(trans-3-(aminomethyl)cyclobutyl)-3-cyclopropyl-1H-pyrazol-4-yl)-1H-pyrazolo[4,3-c]pyridin-1-yl)cyclobutyl)carbamate

A mixture of tert-butyl (trans-3-(6-chloro-1H-pyrazolo[4,3-c]pyridin-1-yl)cyclobutyl)carbamate (35 mg, 108.43 μmol), (trans-3-(3-cyclopropyl-4-(4,4,5,5-tetramethyl-1,3,2-dioxaborolan-2-yl)-1H-pyrazol-1-yl)cyclobutyl)methanamine (120 mg, 378.27 μmol), XPhos Pd G2 (20 mg, 108.43 μmol) and K₃PO₄ (80 mg, 377.36 μmol) in dioxane (5 mL) and water (1.5 mL) was heated at 100 [symbol] under N₂ overnight. The mixture was concentrated and the residue was purified by reverse phase chromatography to afford the title compound (25 mg, yield: 48%) as a light brown oil. MS (ESI) m/z=239.8 [M+H]⁺.

### Step 2. Synthesis of 5-(((trans-3-(4-(1-(trans-3-aminocyclobutyl)-1H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione

A mixture of tert-butyl (trans-3-(6-(1-(trans-3-(aminomethyl)cyclobutyl)-3-cyclopropyl-1H-pyrazol-4-yl)-1H-pyrazolo[4,3-c]pyridin-1-yl)cyclobutyl)carbamate (25 mg, 44.49 μmol), 2-(2,6-dioxo-3-piperidyl)-5-fluoro-isoindoline-1,3-dione (20 mg, 72.41 μmol) and DIPEA (5.75 mg, 44.49 μmol, 7.35 μL) in DMSO (1 mL) was heated at 140 [symbol] for 5 h under N₂. The mixture was cooled to rt and purified by prep-HPLC to give a yellow solid, which was dissolved in DCM (2 mL). TFA (1 mL) was added. The mixture was stirred for 0.5 h, before it was concentrated to afford the title compound (2.3 mg, yield: 8%) as a yellow solid. MS (ESI) m/z=634.5 [M+H]⁺.

### Example 135. 5-(((Trans-3-(3-cyclopropyl-4-(1H-pyrazolo[3,4-b]pyridin-1-yl)-1H-pyrazol-1-yl)cyclobutyl)methyl)amino)-2-(2,6-dioxopiperidin-3-yl)isoindoline-1,3-dione (GS-777)

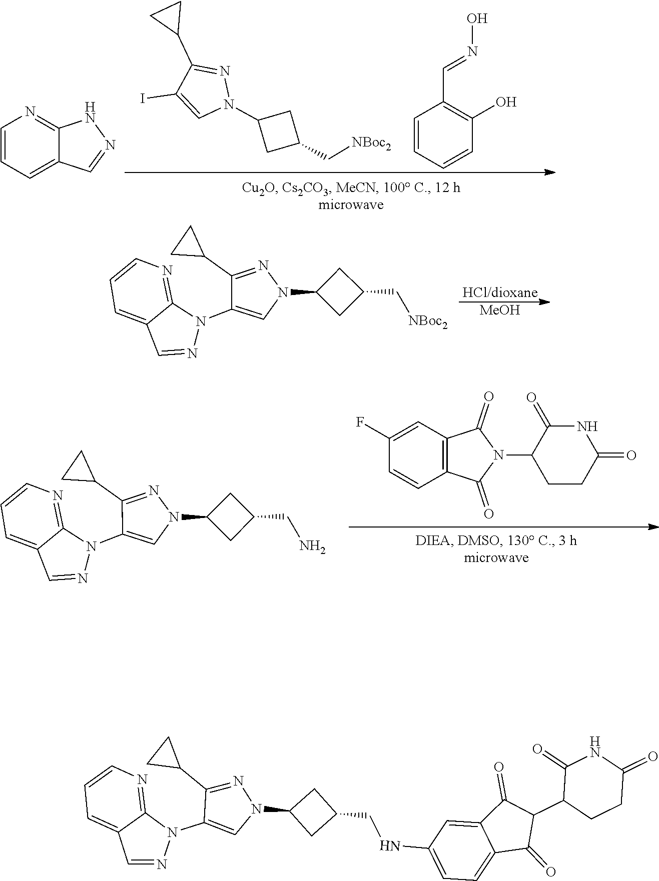

### Step 1. Synthesis of tert-butyl (tert-butoxycarbonyl)((trans-3-(3-cyclopropyl-4-(1H-pyrazolo[3,4-b]pyridin-1-yl)-1-pyrazol-1-yl)cyclobutyl)methyl)carbamate

To a solution of 1H-pyrazolo[3,4-b]pyridine (300 mg, 2.52 mmol) and tert-butyl (tert-butoxycarbonyl)((trans-3-(3-cyclopropyl-4-iodo-1H-pyrazol-1-yl)cyclobutyl)methyl)carbamate (1.56 g, 3.02 mmol) in MeCN (5.0 mL) were added Cu₂O (36.04 mg, 251.84 μmol), 2-hydroxybenzaldehyde oxime (69.07 mg, 503.68 μmol) and Cs₂CO₃ (1.23 g, 3.78 mmol). The reaction was stirred at 100 [symbol] for 12 h under microwave irradiation, before it was filtered. The filtrate was concentrated. The residue was purified by prep-TLC (EtOAc:petroleum ether=1:2) to afford the title compound (13.7 mg, yield: 1%) as an oil. MS (ESI) m/z=509.8 [M+H]⁺.

### Step 2. Synthesis of (trans-3-(3-cyclopropyl-4-(1H-pyrazolo[3,4-b]pyridin-1-yl)-1H-pyrazol-1-yl)cyclobutyl)methanamine

To a solution of tert-butyl (tert-butoxycarbonyl)((trans-3-(3-cyclopropyl-4-(1H-pyrazolo[3,4-b]pyridin-1-yl)-1H-pyrazol-1-yl)cyclobutyl)methyl)carbamate (13.7 mg, 26.94 μmol) in MeOH (1.0 mL) was added HCl (4 M in dioxane, 2.0 mL, 8 mmol). The reaction mixture was stirred at rt for 2 h, before it was concentrated to afford the title compound (11 mg, yield: 98%) as a white solid. MS (ESI) m/z=309.4 [M+H]⁺.

### Step 3. Synthesis of 5-(((trans-3-(3-cyclopropyl-4-(1H-pyrazolo[3,4-b]pyridin-1-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(1H-pyrazolo[3,4-b]pyridin-1-yl)-1H-pyrazol-1-yl)cyclobutyl)methanamine (11 mg, 35.67 μmol), 2-(2,6-dioxo-3-piperidyl)-5-fluoro-isoindoline-1,3-dione (16 mg, 57 μmol) in DMSO was added DIPEA (46.10 mg, 356.70 μmol, 58.95 μL). The reaction mixture was stirred at 130 [symbol] for 3 h under microwave irradiation, before it was concentrated and purified by prep-HPLC to afford the title compound (5.59 mg, yield: 14%) as a white solid. MS (ESI) m/z=565.5 [M+H]⁺.

### Example 136. 5-(((Trans-3-(4-(1-(cis-3-aminocyclobutyl)-1H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione (GS-778)

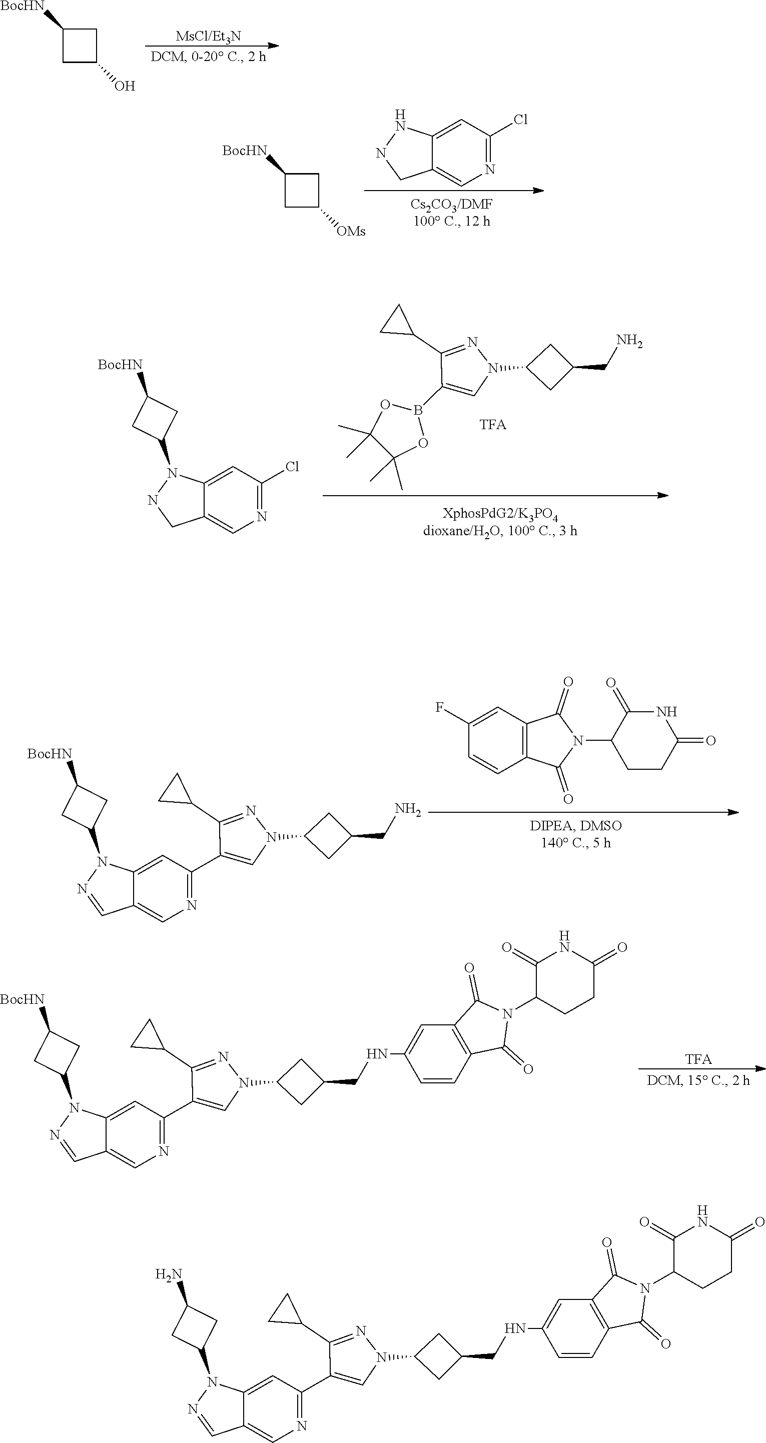

### Step 1. Synthesis of trans-3-((tert-butoxycarbonyl)amino)cyclobutyl methanesulfonate

To a mixture of tert-butyl (trans-3-hydroxycyclobutyl)carbamate (300 mg, 1.60 mmol) and Et₃N (162.1 mg, 1.60 mmol) in DCM (10 mL) was added MsCl (367.0 mg, 3.20 mmol) at 0 [symbol] . The mixture was stirred at 25 [symbol] for 2 h. The resulting mixture was diluted with DCM (20 mL) and washed with sat. Na₂CO₃ solution (5 mL×3). The combined organic layers were washed with brine (10 mL), dried over anhydrous Na₂SO₄, filtered and concentrated in vacuo to afford the title compound (crude, 469 mg) as a yellow solid, which was used directly in the next step.

### Step 2. Synthesis of tert-butyl (trans-3-(6-chloro-1H-pyrazolo[4,3-c]pyridin-1-yl)cyclobutyl)carbamate

A mixture of trans-3-((tert-butoxycarbonyl)amino)cyclobutyl methanesulfonate (469 mg, 1.77 mmol), 6-chloro-1H-pyrazolo[4,3-c]pyridine (257.8 mg, 1.68 mmol) and Cs₂CO₃ (1.15 g, 3.54 mmol) in DMF (10 mL) was stirred at 100 [symbol] for 12 h. The mixture was diluted with EtOAc (30 mL) and washed with H₂O (10 mL×3) and brine (10 mL), dried over anhydrous Na₂SO₄, and filtered. The filtration was concentrated in vacuo. The residue was purified by silica gel chromatography (EtOAc:petroleum ether=0:1 to 3:7) to afford the title compound (140 mg, yield: 25%) as a yellow solid. ¹H NMR (400 MHz, CDCl₃) δ 8.94 (d, J=1.1 Hz, 1H), 8.17 (s, 1H), 7.59 (s, 1H), 5.02 (s, 1H), 4.75 (dd, J=15.9, 8.1 Hz, 1H), 4.15 (d, J=7.9 Hz, 1H), 3.09-3.03 (m, 2H), 2.72 (d, J=9.1 Hz, 2H), 1.46 (s, 9H).

### Step 3. Synthesis of tert-butyl (trans-3-(6-(1-(cis-3-(aminomethyl)cyclobutyl)-3-cyclopropyl-1H-pyrazol-4-yl)-1H-pyrazolo[4,3-c]pyridin-1-yl)cyclobutyl)carbamate

A mixture of tert-butyl (trans-3-(6-chloro-1H-pyrazolo[4,3-c]pyridin-1-yl)cyclobutyl) (40 mg, 123.92 μmol), (trans-3-(3-cyclopropyl-4-(4,4,5,5-tetramethyl-1,3,2-dioxaborolan-2-yl)-1H-pyrazol-1-yl)cyclobutyl)methanamine (TFA salt, 150 mg, 348.64 μmol), XPhos Pd G2 (10 mg, 12.73 μmol) and K₃PO₄ (79 mg, 372.29 μmol) in dioxane (6 mL) and H₂O (2 mL) was stirred at 100 [symbol] for 3 h under N₂. The mixture was concentrated in vacuo. The residue was purified by reverse phase chromatography (0-80% methanol in water) to afford the title compound (30 mg, yield: 51%) as a yellow oil. MS (ESI) m/z=478.3 [M+H]⁺.

### Step 4. Synthesis of tert-butyl (trans-3-(6-(3-cyclopropyl-1-(cis-3-(((2-(2,6-dioxopiperidin-3-yl)-1,3-dioxoisoindolin-5-yl)amino)methyl)cyclobutyl)-1H-pyrazol-4-yl)-1H-pyrazolo[4,3-c]pyridin-1-yl)cyclobutyl)carbamate

A mixture of tert-butyl (trans-3-(6-(1-(cis-3-(aminomethyl)cyclobutyl)-3-cyclopropyl-1H-pyrazol-4-yl)-1H-pyrazolo[4,3-c]pyridin-1-yl)cyclobutyl)carbamate (30.00 mg, 62.81 μmol), 2-(2,6-dioxo-3-piperidyl)-5-fluoro-isoindoline-1,3-dione (35 mg, 126.71 μmol) and DIEA (40.52 mg, 314.07 μmol) in DMSO (1 mL) was stirred for 5 h at 140 [symbol] . The mixture was purified by prep-HPLC (0.05% TFA) to afford the title compound (4.8 mg, yield: 10%) as a yellow oil. MS (ESI) m/z=734.3 [M+H]⁺.

### Step 5. Synthesis of 5-(((trans-3-(4-(1-(cis-3-aminocyclobutyl)-1H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione

To a solution of tert-butyl (trans-3-(6-(3-cyclopropyl-1-(cis-3-(((2-(2,6-dioxopiperidin-3-yl)-1,3-dioxoisoindolin-5-yl)amino)methyl)cyclobutyl)-1H-pyrazol-4-yl)-1H-pyrazolo[4,3-c]pyridin-1-yl)cyclobutyl)carbamate (4.8 mg, 6.54 μmol) in DCM (3 mL) was added TFA (6.54 μmol, 1 mL) at 15 [symbol] . The mixture was stirred at 15 [symbol] for 2 h, before it was concentrated in vacuo. The residue was purified by prep-HPLC (0.05% TFA) to afford the title compound (TFA salt, 2.5 mg, yield: 51%) as a yellow solid. MS (ESI) m/z=634.5 [M+H]⁺.

### Examples 137 and 138. 5-(((Trans-3-(3-cyclopropyl-4-(1-(cis-4-hydroxycyclohexyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-779) and 5-(((trans-3-(3-cyclopropyl-4-(1-(trans-4-hydroxycyclohexyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-782)

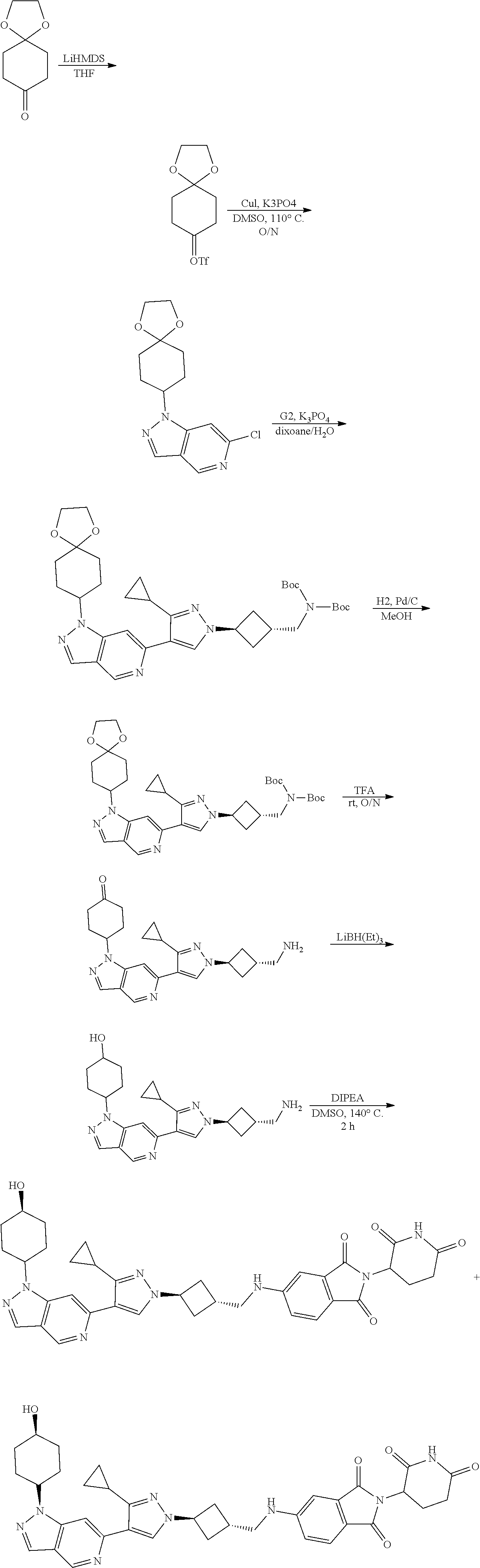

### Step 1. Synthesis of 1,4-dioxaspiro[4.5]dec-7-en-8-yl trifluoromethanesulfonate

To a solution of 1,4-dioxaspiro[4.5]decan-8-one (5.0 g, 32 mmol) in THF (80 mL) was added LiHMDS (5.3 g, 32 mmol) under N₂ at −70 [symbol] . The reaction was stirred at this temperature for 30 min, before Tf₂O (8.1 g, 32 mmol) was added. The mixture was stirred at −70 [symbol] for 1 h, before it was quenched with aqueous NH₄Cl solution. The mixture was extracted with EtOAc. The organic layer was washed with water and brine, dried over Na₂SO₄, filtered and concentrated. The residue was purified by silica gel chromatography (0-10% petroleum ether in EtOAc) to afford the title compound (7.0 g, yield: 76%) as a bright oil.

### Step 2. Synthesis of 6-chloro-1-(1,4-dioxaspiro[4.5]dec-7-en-8-yl)-1H-pyrazolo[4,3-c]pyridine

To a mixture of 6-chloro-1H-pyrazolo[4,3-c]pyridine (1.0 g, 6.5 mmol) and 1,4-dioxaspiro[4.5]dec-7-en-8-yl trifluoromethanesulfonate (5.6 g, 19.5 mmol) in DMSO (20 mL) were added CuI (2.5 g, 13.0 mmol), K₃PO₄ (2.76 g, 13.0 mmol) and trans-cyclohexane-1,2-diamine (372 mg, 3.3 mmol). The reaction mixture was stirred at 110 [symbol] for 6 h under N₂, before it was diluted with MeOH. The mixture was filtered. The filtrate was concentrated and the resulting residue was purified by reverse phase chromatography (0-70% MeCN in H₂O), followed by silica gel chromatography (0-70% petroleum ether in EtOAc) to afford the title compound (200 mg, yield: 11%) as a bright oil. MS (ESI) m/z=292.1 [M+H]⁺.

### Step 3. Synthesis of tert-butyl ((trans-3-(4-(1-(1,4-dioxaspiro[4.5]dec-7-en-8-yl)-1H-pyrazolo[4,3-c]pyridin-6-yl)-3-cyclopropyl-1H-pyrazol-1-yl)cyclobutyl)methyl)(tert-butoxycarbonyl)carbamate

To a mixture of 6-chloro-1-(1,4-dioxaspiro[4.5]dec-7-en-8-yl)-1H-pyrazolo[4,3-c]pyridine (150 mg, 514 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (346 mg, 668 μmol) in water (1 mL) and dioxane (4 mL) were added K₃PO₄ (109 mg, 514 μmmol) and chloro(2-dicyclohexylphosphino-2′,4′,6′-triisopropyl-1,1′-biphenyl)[2-(2′-amino-1,1′-biphenyl)]palladium(II) (404 mg, 514 μmmol). After the mixture was stirred at 90 [symbol] for 2 h, it was quenched with water, and extracted with EtOAc. The organic layer was washed with water and brine, dried over Na₂SO₄, and filtered. The filtration was concentrated. The residue was purified by silica gel chromatography (0-10% petroleum ether in EtOAc) to afford the title compound (200 mg, yield: 60%) as a bright oil. MS (ESI) m/z=647.4 [M+H]⁺.

### Step 4. Synthesis of tert-butyl ((trans-3-(4-(1-(1,4-dioxaspiro[4.5]decan-8-yl)-1H-pyrazolo[4,3-c]pyridin-6-yl)-3-cyclopropyl-1H-pyrazol-yl)cyclobutyl)methyl)(tert-butoxycarbonyl)carbamate

A solution of tert-butyl ((trans-3-(4-(1-(1,4-dioxaspiro[4.5]dec-7-en-8-yl)-1H-pyrazolo[4,3-c]pyridin-6-yl)-3-cyclopropyl-1H-pyrazol-1-yl)cyclobutyl)methyl)(tert-butoxycarbonyl)carbamate (200 mg, 309 μmol) in MeOH (5 mL) was added Pd/C (20 mg, 165 μmol). The mixture was stirred at rt for 6 h and filtered to afford the title compound (200 mg, yield: 100%) as a bright oil. MS (ESI) m/z=649.4 [M+H]⁺.

### Step 5. Synthesis of 4-(6-(1-(trans-3-(aminomethyl)cyclobutyl)-3-cyclopropyl-1H-pyrazol-4-yl)-1H-pyrazolo[4,3-c]pyridin-1-yl)cyclohexan-1-one

A solution of tert-butyl ((trans-3-(4-(1-(1,4-dioxaspiro[4.5]decan-8-yl)-1H-pyrazolo[4,3-c]pyridin-6-yl)-3-cyclopropyl-1H-pyrazol-1-yl)cyclobutyl)methyl)(tert-butoxycarbonyl)carbamate (200 mg, 308 μmol) in TFA (2 mL) and DCM (1 mL) was stirred at rt overnight. The mixture was concentrated and purified by reverse phase chromatography (0-70% MeCN in H₂O) to afford the title compound (120 mg, yield: 96%) as a bright oil. MS (ESI) m/z=405.3 [M+H]⁺.

### Step 6. Synthesis of 4-(6-(1-(trans-3-(aminomethyl)cyclobutyl)-3-cyclopropyl-1H-pyrazol-4-yl)-1-pyrazolo[4,3-c]pyridin-1-yl)cyclohexan-1-ol

To a solution of 4-(6-(1-(trans-3-(aminomethyl)cyclobutyl)-3-cyclopropyl-1H-pyrazol-4-yl)-1H-pyrazolo[4,3-c]pyridin-1-yl)cyclohexan-1-one (120 mg, 296 μmol) in THF (4 mL) was added LiBH(Et)₃ (46 mg, 445 μmol) under 0° C. The reaction mixture was stirred at rt for 1 h before it was quenched with water and filtered. The filtrate was purified by reverse phase chromatography (0-70% MeCN in H₂O) to afford the title compound (60 mg, yield: 50%) as a bright oil. MS (ESI) m/z=407.3 [M+H]⁺.

### Step 7. Synthesis of 5-(((trans-3-(3-cyclopropyl-4-(1-(4-hydroxycyclohexyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

To a mixture of 4-(6-(1-(trans-3-(aminomethyl)cyclobutyl)-3-cyclopropyl-1H-pyrazol-4-yl)-1H-pyrazolo[4,3-c]pyridin-1-yl)cyclohexan-1-ol (60 mg, 147 μmol) and 2-(2,6-dioxo-3-piperidyl)-5-fluoro-isoindoline-1,3-dione (61 mg, 221 μmol) in DMSO (2 mL) was added DIPEA (57 mg, 443 μmol). The mixture was stirred at 140 [symbol] for 2 h and purified by reverse phase chromatography (0-70% MeCN in H₂O) to afford the title compounds.

5-(((Trans-3-(3-cyclopropyl-4-(1-(cis-4-hydroxycyclohexyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (1.2 mg, yield: 1%) as a yellow solid. MS (ESI) m/z=556.3 [M+H]⁺.

5-(((Trans-3-(3-cyclopropyl-4-(1-(trans-4-hydroxycyclohexyl)-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (0.8 mg, yield: 1%) as a yellow solid. MS (ESI) m/z=556.3 [M+H]⁺.

### Example 139. 5-(((Trans-3-(3-cyclopropyl-4-(7-hydroxy-6,7-dihydro-5H-cyclopenta[b]pyridin-2-yl)-1H-pyrazol-1-yl)cyclobutyl)methyl)amino)-2-(2,6-dioxopiperidin-3-yl)isoindoline-1,3-dione (GS-780)

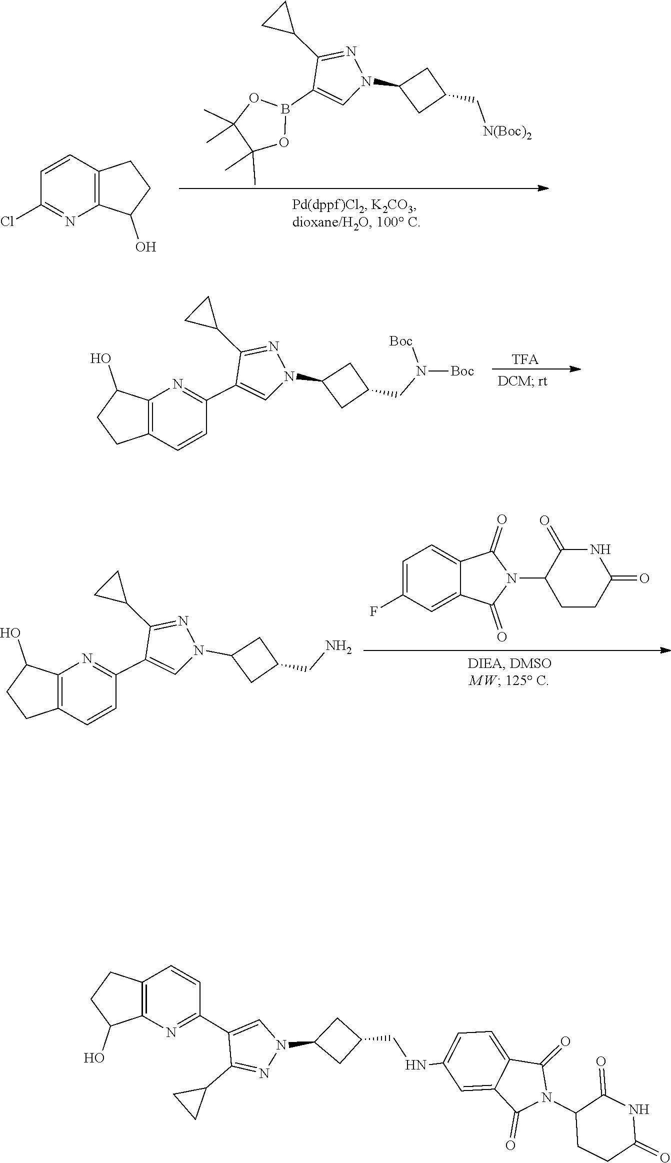

### Step 1. Synthesis of tert-butyl (tert-butoxycarbonyl)((trans-3-(3-cyclopropyl-4-(7-hydroxy-6,7-dihydro-5H-cyclopenta[b]pyridin-2-yl)-1H-pyrazol-1-yl)cyclobutyl)methyl)carbamate

To a stirred solution of 2-chloro-6,7-dihydro-5H-cyclopenta[b]pyridin-7-ol (100.0 mg, 547.7 μmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (457.6 mg, 884.3 μmol) in 1,4-dioxane (10 mL) and H₂O (1 mL) were added XPhos Pd G2 (46.3 mg, 58.9 μmol) and K₃PO₄ (376.7 mg, 1.77 mmol). The mixture was heated to 100 [symbol] and stirred overnight under Ar. The mixture was diluted with water (50 mL) and extracted with ethyl acetate (50 mL×3). The combined organic layers were washed with brine, dried over Na₂SO₄, and filtered. The filtration was concentrated. The residue was purified by silica gel chromatography to afford the title compound (56.1 mg, yield: 18%) as a solid. MS (ESI) m/z=525.4 [M+H]⁺.

### Step 2 to 3. Synthesis of 5-(((trans-3-(3-cyclopropyl-4-(7-hydroxy-6,7-dihydro-5H-cyclopenta[b]pyridin-2-yl)-1H-pyrazol-1-yl)cyclobutyl)methyl)amino)-2-(2,6-dioxopiperidin-3-yl)isoindoline-1,3-dione

GS-780 was synthesized following the procedures for steps 3 to 4 of GS-752 (8.3 mg, yield: 20% over 2 steps) as a yellow solid. MS (ESI) m/z=581.7 [M+H]⁺.

### Example 140. 5-(((Trans-3-(3-cyclopropyl-4-(1-methyl-1H-pyrazolo[4,3-c]pyridin-7-yl)-1H-pyrazol-1-yl)cyclobutyl)methyl)amino)-2-(2,6-dioxopiperidin-3-yl)isoindoline-1,3-dione (GS-781)

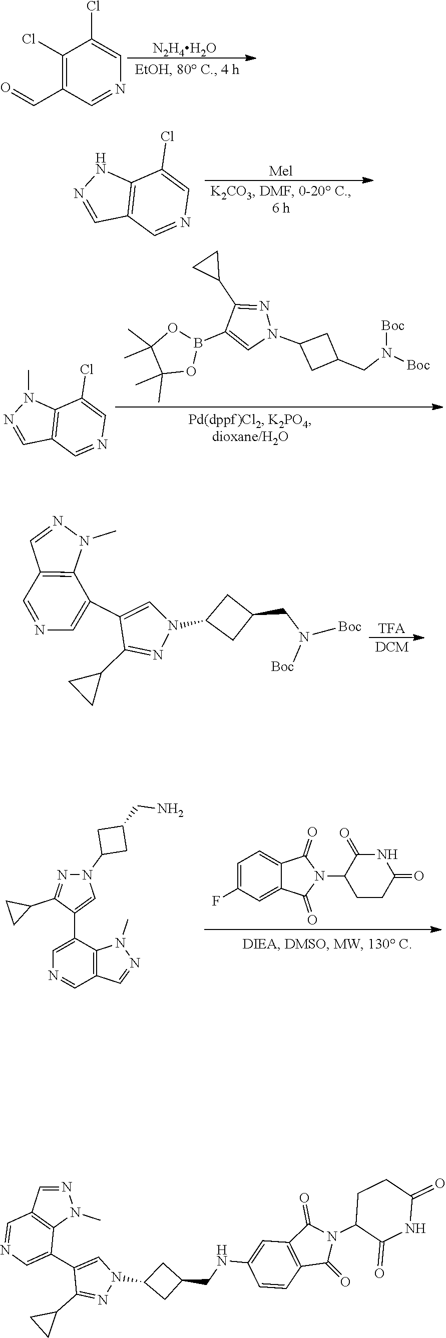

### Step 1. Synthesis of 7-chloro-1H-pyrazolo[4,3-c]pyridine

To a solution of 4,5-dichloropyridine-3-carbaldehyde (5.00 g, 28.4 mmol) in EtOH (50 mL) was added N₂H₂O (6.18 g, 123 mmol, 6 mL) at 20 [symbol] . After stirring at 80 [symbol] for 4 h, the reaction mixture was cooled to 20 [symbol] and poured into H₂O (250 mL). The mixture was then cooled to 0 [symbol] and stirred 30 min. After filtration, the filter cake was washed with ice/water (60 g) and EtOH (10 mL). The solid was dried under reduced pressure to afford the desired product (2.40 g, 15.6 mmol) as off-white solid. ¹H NMR (400 MHz, DMSO-d₆) δ 14.14 (br s, 1H), 9.07 (s, 1H), 8.44 (s, 1H), 8.40 (s, 1H).

### Step 2. Synthesis of 7-chloro-1-methyl-pyrazolo [4,3-c]pyridine

To a solution of 7-chloro-1H-pyrazolo[4,3-c]pyridine (1.20 g, 7.81 mmol) in DMF (10 mL) was added K₂CO₃ (1.08 g, 7.81 mmol) at 0 [symbol] . The mixture was stirred at 0 [symbol] for 30 min, before it was MeI (1.23 g, 8.67 mmol, 540 μL) was added. After stirring at 20 [symbol] for 6 h, the reaction mixture was poured into H₂O (40 mL) and extracted with EtOAc (50 mL×2). The organic layer was washed with brine (50 mL), dried over Na₂SO₄, filtered and concentrated. The residue was purified by silica gel chromatography (petroleum ether:ethyl acetate=50:1 to 1:1) to afford the title compound (0.52 g, yield: 40%) as a yellow solid. ¹H NMR (400 MHz, DMSO-d₆) δ 9.03 (s, 1H), 8.38 (s, 2H), 4.31 (s, 3H). MS (ESI) m/z=168.1 [M+H]⁺.

### Step 3. Synthesis of tert-butyl (tert-butoxycarbonyl)((trans-3-(3-cyclopropyl-4-(1-methyl-1H-pyrazolo[4,3-c]pyridin-7-yl)-1H-pyrazol-1-yl)cyclobutyl)methyl)carbamate

To a solution of 7-chloro-1-methyl-1H-pyrazolo[4,3-c]pyridine (200 mg, 1.19 mmol) and tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (927 mg, 1.79 mmol) in 1,4-dioxane (30 mL) and H₂O (6 mL) were added Pd(dppf)Cl₂ (192 mg, 0.24 mmol) and K₂CO₃ (492 mg, 3.57 mmol). After stirring at 90 [symbol] for 3 h under N₂. The resulting mixture was concentrated under reduced pressure. The residue was purified by silica gel chromatography to afford the title compound (350 mg, yield: 56%) as a colorless oil. MS (ESI) m/z=523.65 [M+H]⁺.

### Step 4. Synthesis of (trans-3-(3-cyclopropyl-4-(1-methyl-1H-pyrazolo[4,3-c]pyridin-7-yl)-1H-pyrazol-1-yl)cyclobutyl)methanamine

To a solution of tert-butyl (tert-butoxycarbonyl)((trans-3-(3-cyclopropyl-4-(1-methyl-1H-pyrazolo[4,3-c]pyridin-7-yl)-1H-pyrazol-1-yl)cyclobutyl)methyl)carbamate (350 mg, 0.67 mmol) in DCM (4 mL) was added TFA (2 mL). The reaction mixture was stirred at rt for 1 h, before it was concentrated. The residue was purified by reverse phase chromatography to afford the title compound (150 mg, yield: 70%) as a yellow solid. MS (ESI) m/z=323.4 [M+H]⁺.

### Step 5. Synthesis of 5-(((trans-3-(3-cyclopropyl-4-(1-methyl-1H-pyrazolo[4,3-c]pyridin-7-yl)-1H-pyrazol-1-yl)cyclobutyl)methyl)amino)-2-(2,6-dioxopiperidin-3-yl)isoindoline-1,3-dione

To a solution of (trans-3-(3-cyclopropyl-4-(1-methyl-H-pyrazolo[4,3-c]pyridin-7-yl)-1H-pyrazol-1-yl)cyclobutyl)methanamine (50 mg, 0.154 mmol) and 2-(2,6-dioxopiperidin-3-yl)-5-fluoroisoindoline-1,3-dione (47 mg, 0.17 mmol) in DMSO (2 mL) was added KF (89 mg, 1.54 mmol). The reaction mixture was stirred at 140° C. for 1 h under microwave irradiation, before it was purified by reverse phase chromatography to afford the title compound (20 mg, yield: 22%) as a yellow solid. MS (ESI) m/z=579.7 [M+H]⁺.

### Example 141. 5-((2-(6-(3-Cyclopropyl-1H-indazol-1-yl)spiro[3.3]heptan-2-yl)ethyl)amino)-2-(2,6-dioxopiperidin-3-yl)isoindoline-1,3-dione (GS-783)

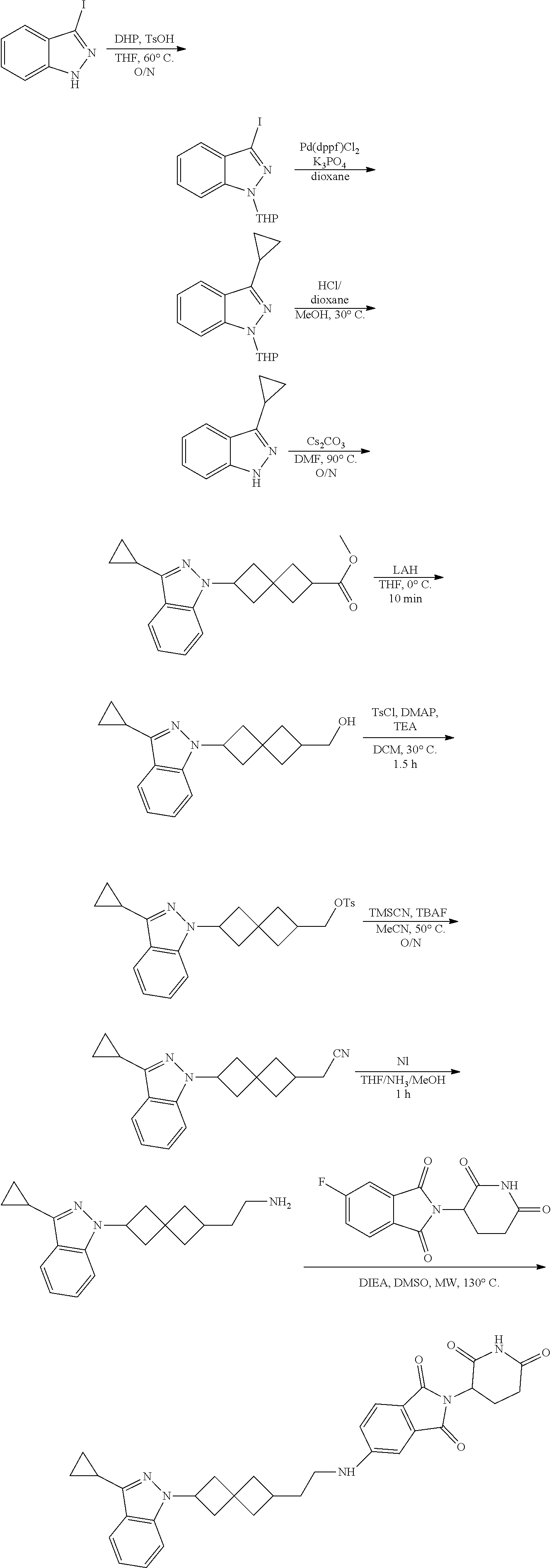

### Step 1 to 2. Synthesis of 3-cyclopropyl-1-(tetrahydro-2H-pyran-2-yl)-1H-indazole

The title compound was synthesized following the procedures for steps 2 to 3 of GS-756 (4.5 g, yield: 91% over 2 steps) as a bright oil. MS (ESI) m/z=243.1 [M+H]⁺.

### Step 3. Synthesis of 3-cyclopropyl-1H-indazole

To a solution of 3-cyclopropyl-1-(tetrahydro-2H-pyran-2-yl)-1H-indazole (4.5 g, 18.5 mmol) in MeOH (10 mL) was added HCl solution (4 M in dioxane, 10 mL). The reaction mixture was stirred at 30 [symbol] for 1 h, before it was concentrated to afford the title compound (2.9 g, yield: 99%) as a white solid. MS (ESI) m/z=159.0 [M+H]⁺.

### Step 4 to 9. Synthesis of 5-((2-(6-(3-cyclopropyl-1H-indazol-1-yl)spiro[3.3]heptan-2-yl)ethyl)amino)-2-(2,6-dioxopiperidin-3-yl)isoindoline-1,3-dione

GS-783 was synthesized following the procedures for steps 3 to 8 of GS-724 (51 mg, yield: 15% over 6 steps) as a green solid. MS (ESI) m/z=552.5 [M+H]⁺.

### Example 142. 5-(((Trans-3-(3-cyclopropyl-4-((1-methyl-1H-pyrazol-5-yl)amino)-1H-pyrazol-1-yl)cyclobutyl)methyl)amino)-2-(2,6-dioxopiperidin-3-yl)isoindoline-1,3-dione (GS-784)

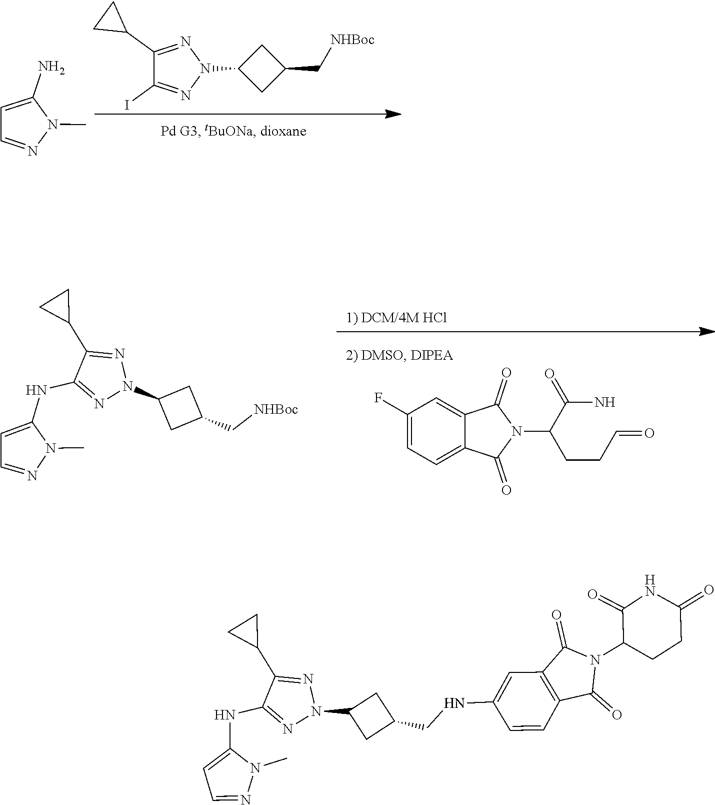

### Step 1. Synthesis of tert-butyl ((trans-3-(3-cyclopropyl-4-((I-methyl-1H-pyrazol-5-yl)amino)-1H-pyrazol-1-yl)cyclobutyl)methyl)carbamate

A mixture of 2-methylpyrazol-3-amine (100 mg, 1.03 mmol), tert-butyl ((trans-3-(3-cyclopropyl-4-iodo-1H-pyrazol-1-yl)cyclobutyl)methyl)carbamate (100 mg, 193.27 μmol), t-BuONa (150 mg, 1.56 mmol) and tBuBrettPhos Pd G3 (20 mg, 21 μmol) in dioxane (2 mL) was heated at 100 [symbol] under N₂ overnight. After cooling to rt, the mixture was filtered and concentrated. The residue was purified by reverse phase chromatography to afford the title compound (10 mg, yield: 2%) as a light brown oil. MS (ESI) m/z=387.5 [M+H]⁺.

### Step 2. Synthesis of 5-(((trans-3-(3-cyclopropyl-4-((1-methyl-1H-pyrazol-5-yl)amino)-1H-pyrazol-1-yl)cyclobutyl)methyl)amino)-2-(2,6-dioxopiperidin-3-yl)isoindoline-1,3-dione

To a solution of tert-butyl ((trans-3-(3-cyclopropyl-4-((1-methyl-1H-pyrazol-5-yl)amino)-1H-pyrazol-1-yl)cyclobutyl)methyl)carbamate (10 mg, 25.87 μmol) in DCM (3 mL) was added HCl solution (4 M in dioxane, 1 mL) at rt. The mixture was stirred for 1 h, before it was concentrated. The above crude intermediate was mixed with DIPEA (5 mg, 38.76 μmol), 2-(2,6-dioxo-3-piperidyl)-5-fluoro-isoindoline-1,3-dione (7.5 mg, 26.83 μmol) and DMSO (1 mL). The mixture was heated at 140 [symbol] for 5 h. The mixture was cooled to rt, before it was purified by prep-HPLC to afford the title compound (3 mg, yield: 21%) as a yellow solid. MS (ESI) m/z=543.7 [M+H]⁺.

### Example 143. 5-(((Trans-3-(4-(2-(cis-3-aminocyclobutyl)-2H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione (GS-785)

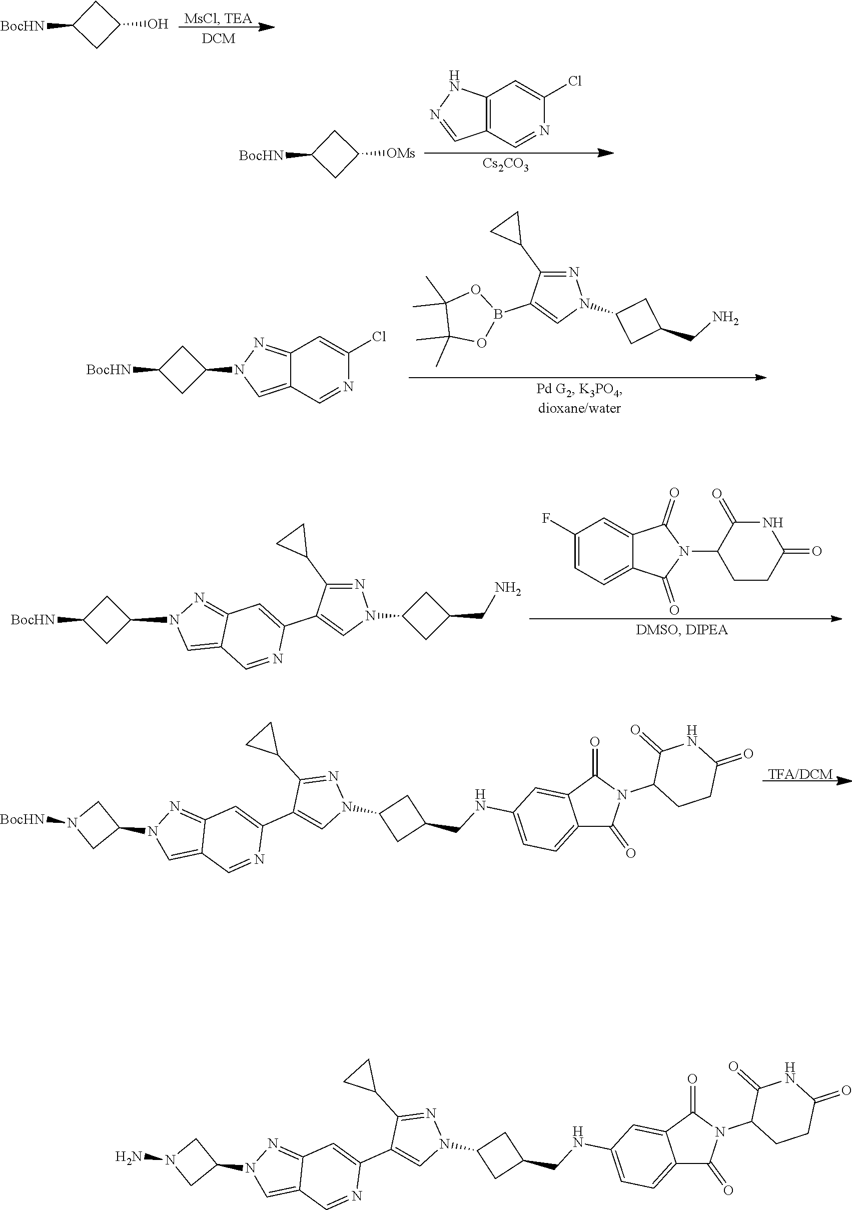

GS-785 was synthesized following the procedures for GS-778 (4 mg, yield: 4% over 5 steps) as a yellow solid. MS (ESI) m/z=634.2 [M+H]⁺.

### Example 144. 5-(((Trans-3-(4-(2-(trans-3-aminocyclobutyl)-2H-pyrazolo[4,3-c]pyridin-6-yl)-3-cyclopropyl-1H-pyrazol-1-yl)cyclobutyl)methyl)amino)-2-(2,6-dioxopiperidin-3-yl)isoindoline-1,3-dione (GS-786)

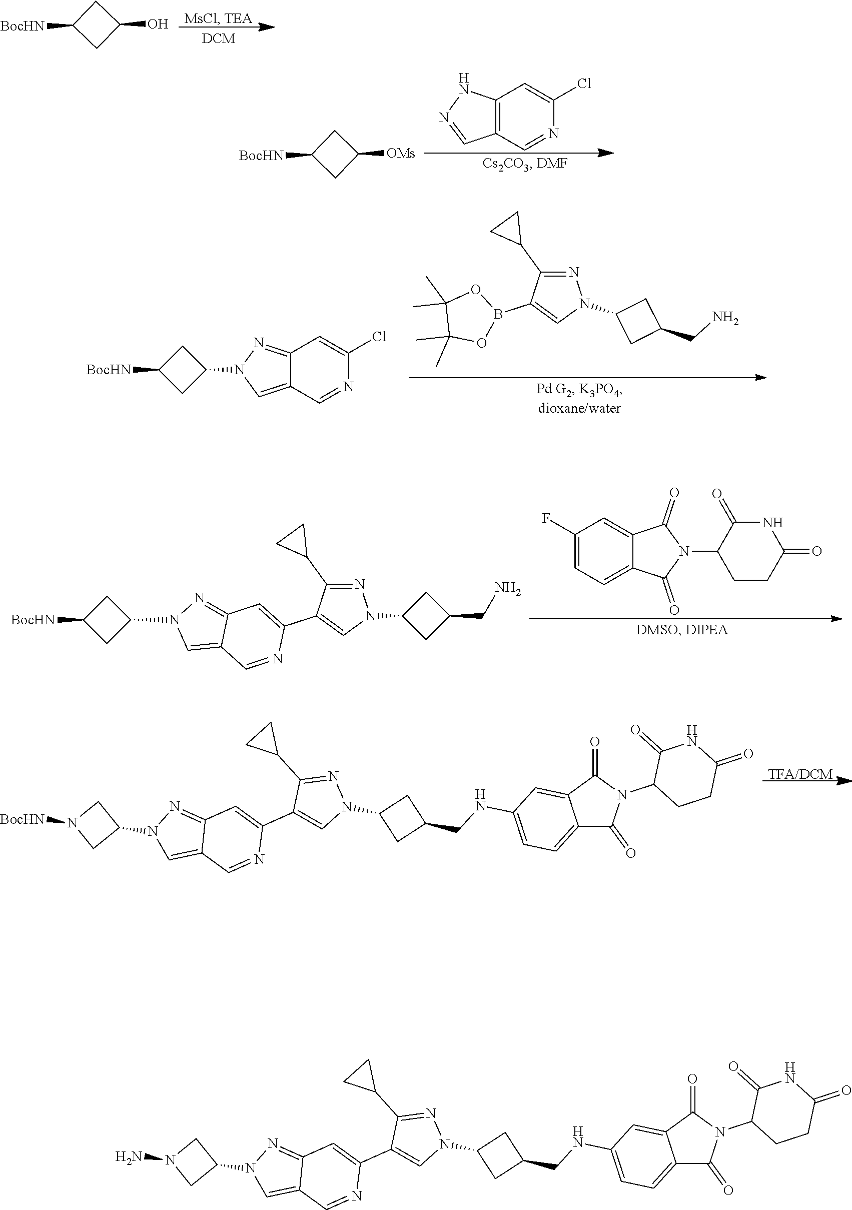

GS-786 was synthesized following the procedures for GS-778 (5 mg, yield: 3% over 5 steps) as a yellow solid. MS (ESI) m/z=634.2 [M+H]⁺.

### Example 145. 3-(5-((Trans-3-(3-cyclopropyl-4-(5-fluoro-6-methylpyridin-2-yl)-1H-pyrazol-1-yl)cyclobutyl)methoxy)-1-oxoisoindolin-2-yl)piperidine-2,6-dione (GS-787)

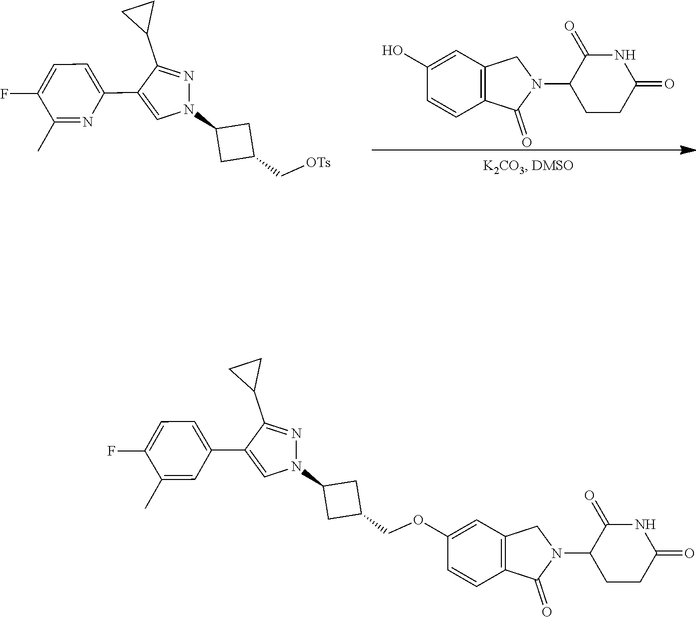 [SMILES: Cc1ccc(S(=O)(=O)OC[C@H]2C[C@H](n3cc(-c4ccc(F)c(C)n4)c(C4CC4)n3)C2)cc1 ; O=C1CCC(N2Cc3cc(O)ccc3C2=O)C(=O)N1 ; Cc1cc(-c2cn([C@H]3C[C@H](COc4ccc5c(c4)CN(C4CCC(=O)NC4=O)C5=O)C3)nc2C2CC2)ccc1F]

To a solution of (trains-3-(3-cyclopropyl-4-(5-fluoro-6-methylpyridin-2-yl)-1H-pyrazol-1-yl)cyclobutyl)methyl 4-methylbenzenesulfonate (30 mg, 65.86 μmol) in DMSO (2 mL) were added 3-(5-hydroxy-1-oxo-isoindolin-2-yl)piperidine-2,6-dione (17.14 mg, 65.86 μmol) and K₂CO₃ (18.18 mg, 131.71 μmol). The mixture was stirred at 70 [symbol] for 2 h, before it was purified by reverse phase chromatography (0.1% TFA in water:MeOH=1:1) to afford the title compound (3.1 mg, yield: 9%) as a white solid. MS (ESI) m/z=644.2 [M+H]⁺.

### Example 146. 3-(5-(((Trans-3-(3-cyclopropyl-4-(5-fluoro-1,7-naphthyridin-8-yl)-1H-pyrazol-1-yl)cyclobutyl)methyl)amino)-1-oxoisoindolin-2-yl)piperidine-2,6-dione (GS-788)

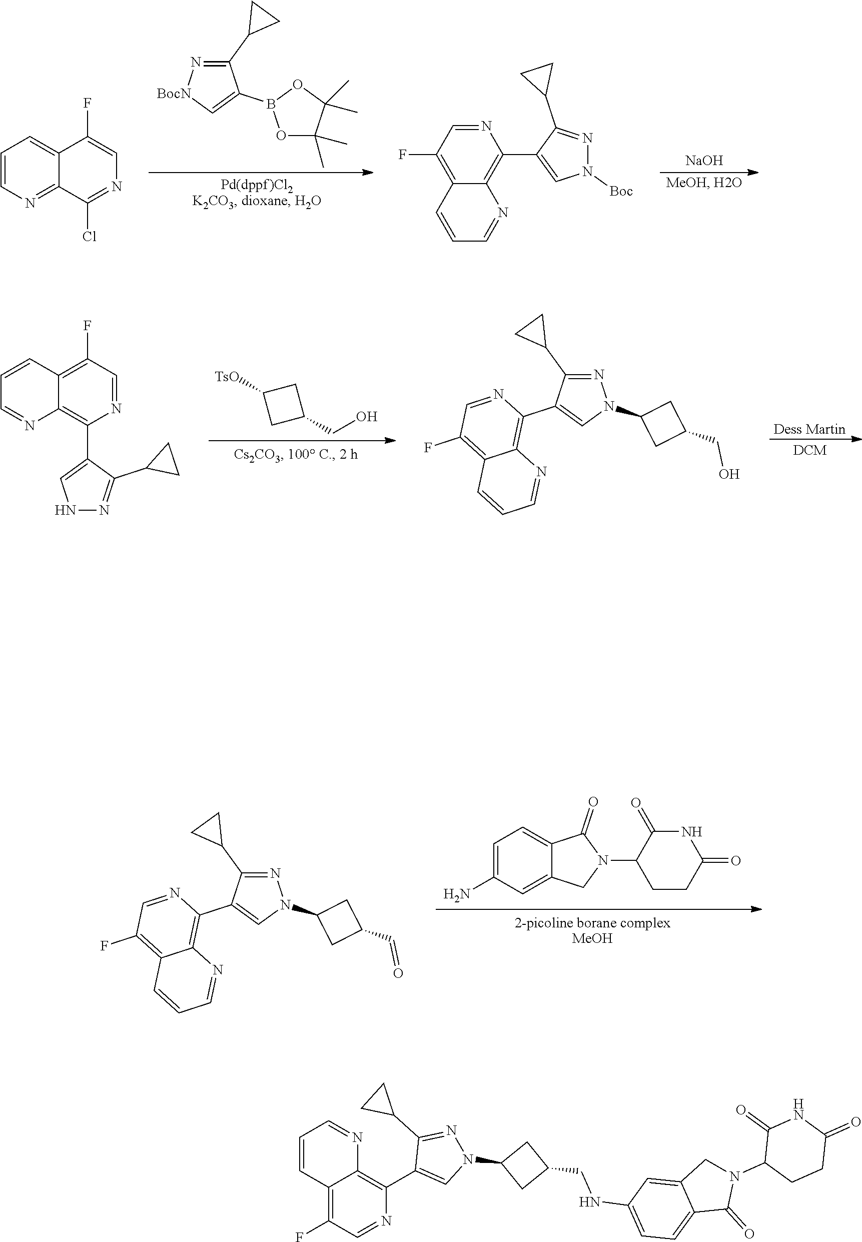

### Step 1. Synthesis of tert-butyl 3-cyclopropyl-4-(5-fluoro-1,7-naphthyridin-8-yl)-1H-pyrazole-1-carboxylate

To a solution of 8-chloro-5-fluoro-1,7-naphthyridine (600 mg, 3.29 mmol) and tert-butyl 3-cyclopropyl-4-(4,4,5,5-tetramethyl-1,3,2-dioxaborolan-2-yl)-1H-pyrazole-1-carboxylate (1.10 g, 3.29 mmol) in dioxane (25 mL) and water (2.5 mL) were added Pd(dppf)Cl₂ (240.2 mg, 328.6 μmol) and K₂CO₃ (1.36 g, 9.86 mmol). The mixture was stirred at 90 [symbol] for 4 h, before it was purified by prep-HPLC to afford the title compound (320.0 mg, yield: 27%). MS (ESI) m/z=355.4 [M+H]⁺.

### Step 2. Synthesis of 8-(3-cyclopropyl-1H-pyrazol-4-yl)-5-fluoro-1,7-naphthyridine

To a solution of tert-butyl 3-cyclopropyl-4-(5-fluoro-1,7-naphthyridin-8-yl)-1H-pyrazole-1-carboxylate (340 mg, 959.43 μmol) in MeOH (15 mL) and water (5 mL) was added NaOH (76.7 mg, 1.92 mmol). The mixture was stirred at rt for 30 min, before it was concentrated. The residue was purified by silica gel chromatography (DCM:MeOH=10:1) to afford the title compound (220.0 mg, yield: 90%). MS(ESI) m/z=255.4 [M+H]⁺.

### Step 3. Synthesis of (trans-3-(3-cyclopropyl-4-(5-fluoro-1,7-naphthyridin-8-yl)-1H-pyrazol-1-yl)cyclobutyl)methanol

To a solution of 8-(3-cyclopropyl-1H-pyrazol-4-yl)-5-fluoro-1,7-naphthyridine (180.0 mg, 707.9 μmol) in DMSO (5 mL) were added cis-3-(hydroxymethyl)cyclobutyl 4-methylbenzenesulfonate (362.9 mg, 1.42 mmol) and Cs₂CO₃ (690.3 mg, 2.12 mmol). The mixture was stirred at 90 [symbol] for 4 h, before it was purified by prep-HPLC to afford the title compound (210.0 mg, yield: 88%). MS (ESI) m/z=339.6 [M+H]⁺.

### Step 4. Synthesis of trans-3-(3-cyclopropyl-4-(5-fluoro-1,7-naphthyridin-8-yl)-1H-pyrazol-1-yl)cyclobutane-1-carbaldehyde

To a solution of (trans-3-(3-cyclopropyl-4-(5-fluoro-1,7-naphthyridin-8-yl)-1H-pyrazol-1-yl)cyclobutyl)methanol (230 mg, 679.71 μmol) in DCM (15 mL) was added Dess-Martin periodinane (720.7 mg, 1.70 mmol). The mixture was stirred at rt for 2.5 h, before it was purified by prep-HPLC to afford the title compound (210.0 mg, yield: 92%). MS (ESI) m/z=337.5 [M+H]⁺.

### Step 5. Synthesis of 3-(5-(((trans-3-(3-cyclopropyl-4-(5-fluoro-1,7-naphthyridin-8-yl)-1H-pyrazol-1-yl)cyclobutyl)methyl)amino)-1-oxoisoindolin-2-yl)piperidine-2,6-dione

To a solution of trans-3-(3-cyclopropyl-4-(5-fluoro-1,7-naphthyridin-8-yl)-1H-pyrazol-1-yl)cyclobutane-1-carbaldehyde (45.0 mg, 133.7 μmol) in MeOH (5 mL) were added 3-(5-amino-1-oxo-isoindolin-2-yl)piperidine-2,6-dione (34.6 mg, 133.7 μmol) and 2-picoline borane complex (42.9 mg, 401.3 μmol). The mixture was stirred at rt for 1.5 h, before it was purified by prep-HPLC to afford the title compound (2.6 mg, yield: 16%). MS (ESI) m/z=580.4 [M+H]⁺.

### Example 147. 5-(((Trans-3-(3-cyclopropyl-4-(2-(trans-4-hydroxycyclohexyl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-789)

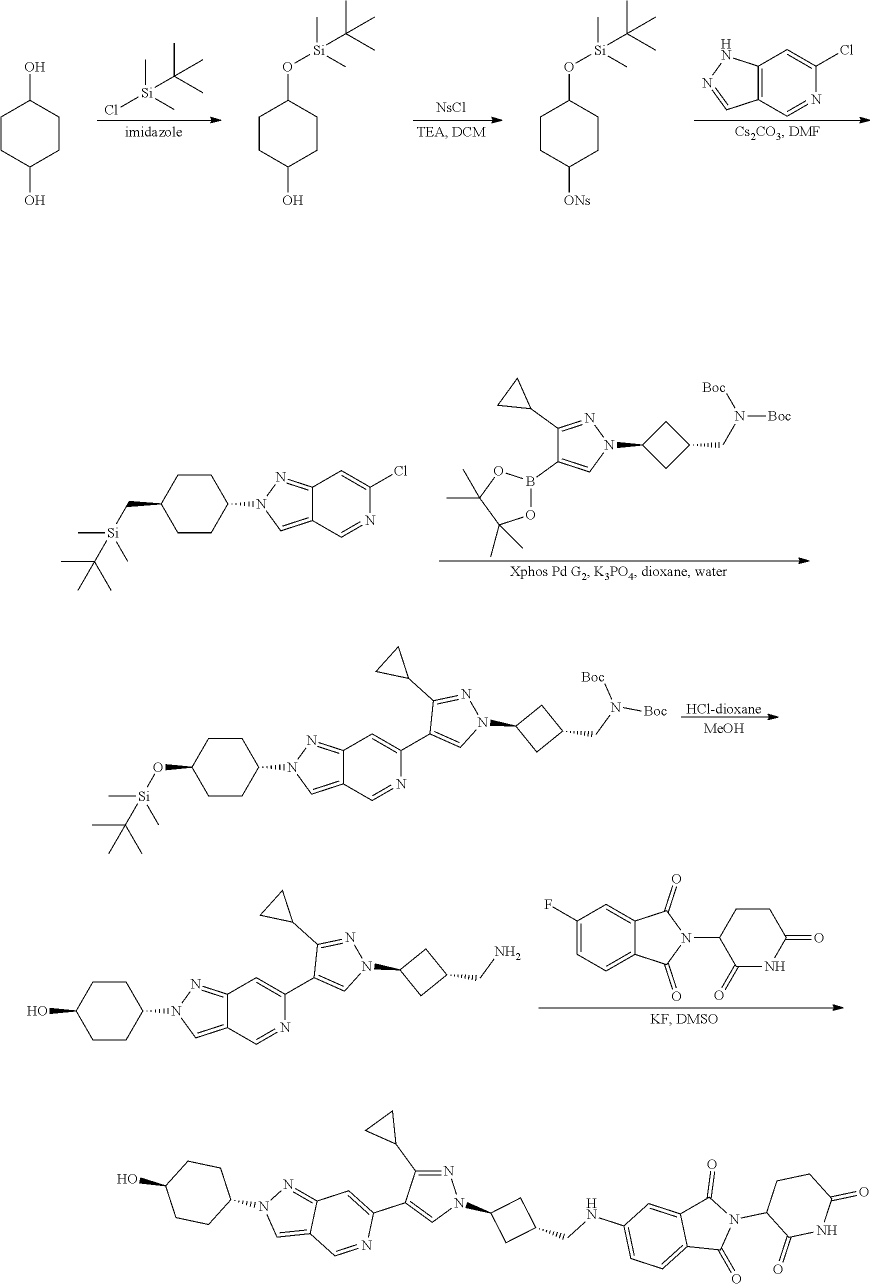

### Step 1. Synthesis of 4-((tert-butyldimethylsilyl)oxy)cyclohexan-1-ol

To a mixture of cyclohexane-1,4-diol (10 g, 86 mmol) and tert-butyl-chloro-dimethyl-silane (13 g, 86 mmol) in THF (150 mL) was added imidazole (5.8 g, 86 mmol) at 0 [symbol] . The mixture was stirred at rt overnight before it was filtered. The filter cake was triturated with EtOAc and petroleum ether to afford the title compound (10 g, yield: 51%) as a colorless oil.

### Step 2. Synthesis of 4-((tert-butyldimethylsilyl)oxy)cyclohexyl 4-nitrobenzenesulfonate

To a solution of 4-((tert-butyldimethylsilyl)oxy)cyclohexan-1-ol (15 g, 65.10 mmol) in DCM (300 mL) were added TEA (19.76 g, 195.30 mmol) and 4-nitrobenzenesulfonyl chloride (17.31 g, 78.12 mmol) at rt. After stirring for 1 h, the mixture was washed with water (100 mL), aq. Na₂CO₃ solution (5%, 100 mL×2) and brine (100 mL). The organic layer was concentrated in vacuo. The residue was triturated in petroleum ether (150 mL) for 0.5 h and filtered. The filter cake was dried in vacuo to afford the title compound (15 g, yield: 55%) as a yellow solid.

### Step 3. Synthesis of 2-(trans-4-((tert-butyldimethylsilyl)methyl)cyclohexyl)-6-chloro-2H-pyrazolo[4,3-c]pyridine

To a mixture of 4-((tert-butyldimethylsilyl)oxy)cyclohexyl 4-nitrobenzenesulfonate (14.40 g, 34.64 mmol) and 6-chloro-11H-pyrazolo[4,3-c]pyridine (3.8 g, 24.74 mmol) in DMSO (100 mL) was added Cs₂CO₃ (16.12 g, 49.49 mmol) at 25 [symbol] . The mixture was stirred at 100 [symbol] for 0.5 h, before it was cooled to 25 [symbol] . The mixture was diluted with water (200 mL) and extracted with ethyl acetate (200 mL). The organic layer was concentrated in vacuo. The residue was purified by silica gel chromatography (petroleum ether:ethyl acetate=100:1 to 1:1) to afford the title compound (700 mg, yield: 8%) as a white solid. MS (ESI) m/z=366.24 [M+H]⁺.

### Step 4. Synthesis of tert-butyl (tert-butoxycarbonyl)((trans-3-(4-(2-(trans-4-((tert-butyldimethylsilyl)oxy)cyclohexyl)-2H-pyrazolo[4,3-c]pyridin-6-yl)-3-cyclopropyl-1H-pyrazol-1-yl)cyclobutyl)methyl)carbamate

A mixture of 2-(trans-4-((tert-butyldimethylsilyl)methyl)cyclohexyl)-6-chloro-2H-pyrazolo[4,3-c]pyridine (300 mg, 819.73 μmol), tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (509.02 mg, 983.68 μmol), K₃PO₄ (521.99 mg, 2.46 mmol) and XPhos Pd G2 (60 mg) in dioxane (9 mL) and water (3 mL) was stirred at 100 [symbol] for 18 h under N₂. The mixture was concentrated in vacuo, and the residue was purified by silica gel chromatography (petroleum ether:ethyl acetate=100:1 to 4:1) to afford the title compound (200 mg, yield: 34%) as a white solid. MS (ESI) m/z=721.40 [M+H]⁺.

### Step 5. Synthesis of trans-4-(6-(1-(trans-3-(aminomethyl)cyclobutyl)-3-cyclopropyl-1H-pyrazol-4-yl)-2H-pyrazolo[4,3-c]pyridin-2-yl)cyclohexan-1-ol

To a solution of tert-butyl (tert-butoxycarbonyl)((trans-3-(4-(2-(trans-4-((tert-butyldimethylsilyl)oxy)cyclohexyl)-2H-pyrazolo[4,3-c]pyridin-6-yl)-3-cyclopropyl-1H-pyrazol-1-yl)cyclobutyl)methyl)carbamate (200 mg, 277.39 μmol) in methanol (10 mL) was added HCl solution (4 M in dioxane, 10 mL) at 25 [symbol] . The mixture was stirred for 1 h, before it was concentrated in vacuo. The residue was dissolved in MeOH (2 mL). Aqueous NaOH solution (1 M) was added to adjust pH to 8. The mixture was purified by reverse phase chromatography to afford the title compound (70 mg, yield: 62%) as a white solid. MS (ESI) m/z=407.34 [M+H]⁺.

### Step 6. Synthesis of 5-(((trans-3-(3-cyclopropyl-4-(2-(trans-4-hydroxycyclohexyl)-2H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

A mixture of trans-4-(6-(1-(trans-3-(aminomethyl)cyclobutyl)-3-cyclopropyl-1H-pyrazol-4-yl)-2H-pyrazolo[4,3-c]pyridin-2-yl)cyclohexan-1-ol (70 mg, 172.19 μmol), 2-(2,6-dioxo-3-piperidyl)-5-fluoro-isoindoline-1,3-dione (66.59 mg, 241.07 μmol) and KF (60.02 mg, 1.03 mmol) in DMSO (5 mL) was stirred at 145 [symbol] for 2 h, before it was cooled to rt. The mixture was purified by reverse phase chromatography to afford the title compound (11.5 mg, yield: 10%) as a yellow solid. MS (ESI) m/z=663.35 [M+H]⁺.

### Example 148. 5-(((Trans-3-(3-cyclopropyl-4-(7-fluoro-2-methyl-2H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-790)

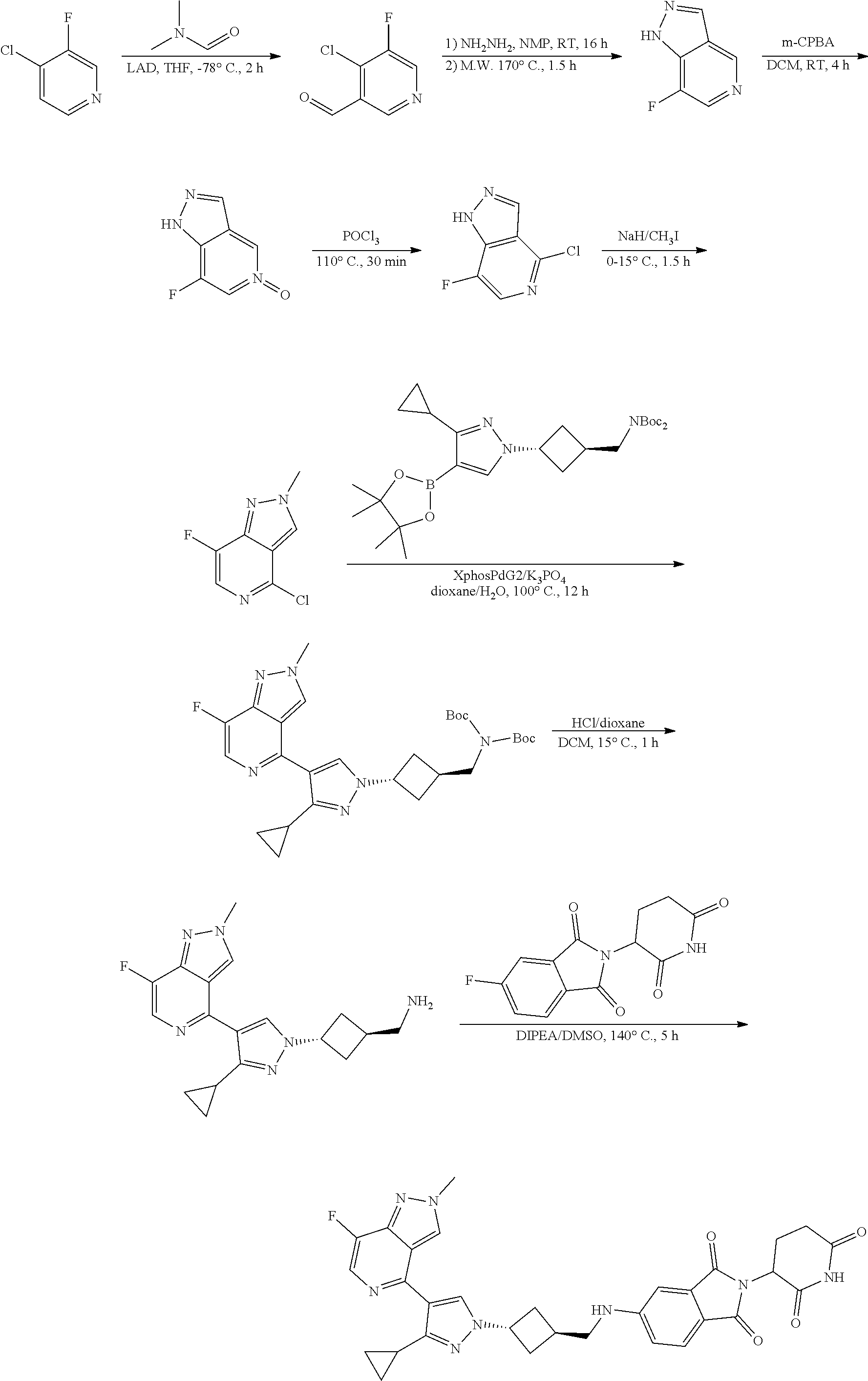

### Step 1. Synthesis of 4-chloro-5-fluoronicotinaldehyde

To a solution of 4-chloro-3-fluoropyridine (25.0 g, 191 mmol) in THF (150 mL) at −78 [symbol] was added lithium diisopropyl amide (2 M in THF, 144 mL, 288 mmol) slowly. After the mixture was stirred at −78 [symbol] for 2 h, DMF (19.2 g, 287 mmol) was added. The reaction was warmed to rt slowly. The mixture was quenched with sat. ammonium chloride solution (200 mL) and extracted with EtOAc (200 mL×2). The combined organic phase was washed with brine, dried over Na₂SO₄, filtered and concentrated in vacuo. The residue was purified by silica gel chromatography (petroleum ether:EtOAc=3:1) to afford the title compound (7.00 g, yield: 23%) as a white solid. MS (ESI) m/z=160.1 [M+H]⁺.

### Step 2. Synthesis of 7-fluoro-1H-pyrazolo[4,3-c]pyridine

A solution of 4-chloro-5-fluoronicotinaldehyde (7.00 g, 44.0 mmol) and hydrazine hydrate (2.64 g, 52.8 mmol) in NMP (35 mL) was stirred at rt for 16 h. The mixture was stirred at 170 [symbol] for 1.5 h under microwave irradiation. After cooling to rt, the reaction mixture was concentrated in vacuo. The residue was purified by prep-HPLC (0.1% NH₄HCO₃) to afford the title compound (2.20 g, yield: 37%) as a brown solid. MS (ESI) m/z=138.1 [M+H]⁺.

### Step 3. Synthesis of 7-fluoro-1H-pyrazolo[4,3-c]pyridine 5-oxide

A solution of 7-fluoro-1H-pyrazolo[4,3-c]pyridine (2.20 g, 16.1 mmol) and m-CPBA (5.54 g, 32.2 mmol) in DCM (20 mL) was stirred at rt for 4 h. The precipitate was filtered and the filter cake was washed with EtOAc (50 mL). The solid was dried in vacuo to afford the title compound (2.10 g, yield: 85%) as a brown solid. MS (ESI) m/z=154.0 [M+H]⁺.

### Step 4. Synthesis of 4-chloro-7-fluoro-1H-pyrazolo[4,3-c]pyridine

A solution of 7-fluoro-1H-pyrazolo[4,3-c]pyridine 5-oxide (2.10 g, 13.7 mmol) in POCl₃ (20 mL) was stirred at 110 [symbol] for 30 min. After cooling to rt, the mixture was concentrated to remove most of POCl₃. The residue was poured into ice-water (100 mL) and adjusted to pH=7 with sat. aqueous NaHCO₃. The mixture was extracted with EtOAc (50 mL×2). The combined organic phase was washed brine, dried over Na₂SO₄, and filtered. The filtrate was concentrated in vacuo. The residue was purified by silica gel chromatography (petroleum ether:EtOAc=2:1) to afford the title compound (350 mg, yield: 15%) as a white solid. ¹H NMR (400 MHz, DMSO-d₆) δ 8.29 (s, 1H), 8.05 (d, J=2.0 Hz, 1H). MS (ESI) m/z=172.0 [M+H]⁺.

### Step 5. Synthesis of 4-chloro-7-fluoro-1H-pvrazolo[4,3-c]pyridine

To a solution of 4-chloro-7-fluoro-1H-pyrazolo[4,3-c]pyridine (100 mg, 582.89 μmol) in DMF (2 mL) was added NaH (60% in mineral oil, 48.00 mg, 1.20 mmol) at 0 [symbol] . After the reaction was stirred for 0.5 h, iodomethane (166.00 mg, 1.17 mmol, 72.81 L) was added at 0° C. The mixture was stirred for 1 h at 15 [symbol] . The reaction was repeated and the reaction mixture was combined. The combined mixture was diluted with EtOAc (30 mL), washed with H₂O (10 mL×3) and brine (10 mL), dried over anhydrous Na₂SO₄, and filtered. The filtrate was concentrated in vacuo. The residue was purified by silica gel chromatography (EtOAc:petroleum ether=0:1 to 3:7) to afford the title compound (51 mg, yield: 310%) as a yellow solid. ¹H NMR (400 MHz, CDCl₃) δ 8.14 (d, J=2.3 Hz, 1H), 7.91 (d, J=3.1 Hz, 1H), 4.30 (s, 3H). MS (ESI) m/z=186.1 [M+H]⁺.

### Step 6. Synthesis of tert-butyl (tert-butoxycarbonyl)((trans-3-(3-cyclopropyl-4-(7-fluoro-2-methyl-2H-pyrazolo[4,3-c]pyridin-4-yl)-1H-pyrazol-1-yl)cyclobutyl)methyl)carbamate

A mixture of 4-chloro-7-fluoro-1H-pyrazolo[4,3-c]pyridine (51 mg, 274.81 μmol), tert-butyl (tert-butoxycarbonyl)((trans-3-(3-cyclopropyl-4-(4,4,5,5-tetramethyl-1,3,2-dioxaborolan-2-yl)-1H-pyrazol-1-yl)cyclobutyl)methyl)carbamate (213 mg, 411.62 μmol), K₃PO₄ (176 mg, 829.41 μmol) and Xphos Pd G2 (22 mg, 28.00 μmol) in H₂O (2 mL) and dioxane (6 mL) was stirred at 100 [symbol] for 12 h under N₂. The mixture was concentrated in vacuo. The residue was purified by reverse phase chromatography (0-80% methanol in water) to afford the title compound (66 mg, yield: 44%) as a yellow oil. MS (ESI) m/z=541.3 [M+H]⁺.

### Step 7. Synthesis of (trans-3-(3-cyclopropyl-4-(7-fluoro-2-methyl-2H-pyrazolo[4,3-c]pyridin-4-yl)-1H-pyrazol-1-yl)cyclobutyl)methanamine

A solution of tert-butyl (tert-butoxycarbonyl)((trans-3-(3-cyclopropyl-4-(7-fluoro-2-methyl-2H-pyrazolo[4,3-c]pyridin-4-yl)-1H-pyrazol-1-yl)cyclobutyl)methyl)carbamate (66 mg, 122.08 μmol) in HCl/dioxane (5 mL) and DCM (1 mL) was stirred for 1 h at 15 [symbol] . The mixture was concentrated in vacuo to afford the title compound (HCl salt, crude, 61 mg), which was used directly in the next step. MS (ESI) m/z=341.3 [M+H]⁺.

### Step 8. Synthesis of 5-(((trans-3-(3-cyclopropyl-4-(7-fluoro-2-methyl-2H-pyrazolo[4,3-c]pyridin-4-yl)-1H-pyrazol-1-yl)cyclobutyl)methyl)amino)-2-(2,6-dioxopiperidin-3-yl)isoindoline-1,3-dione

A mixture of (trans-3-(3-cyclopropyl-4-(7-fluoro-2-methyl-2H-pyrazolo[4,3-c]pyridin-4-yl)-1H-pyrazol-1-yl)cyclobutyl)methanamine (HCl salt, crude, 47.59 μmol), 2-(2,6-dioxo-3-piperidyl)-5-fluoro-isoindoline-1,3-dione (72.63 mg, 262.95 μmol) and DIEA (95.93 mg, 743.65 μmol) in DMSO (2 mL) was stirred for 5 h at 140 [symbol] . The mixture was purified by prep-TLC (dichloromethane:methanol=10:1) to afford the title compound (2.02 mg, yield: 2% over 2 steps) as a yellow solid. MS (ESI) m/z=597.3 [M+H]⁺.

### Example 149. 5-(((Trans-3-(3-Cyclopropyl-4-(7-fluoro-1-isopropyl-1H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-791)

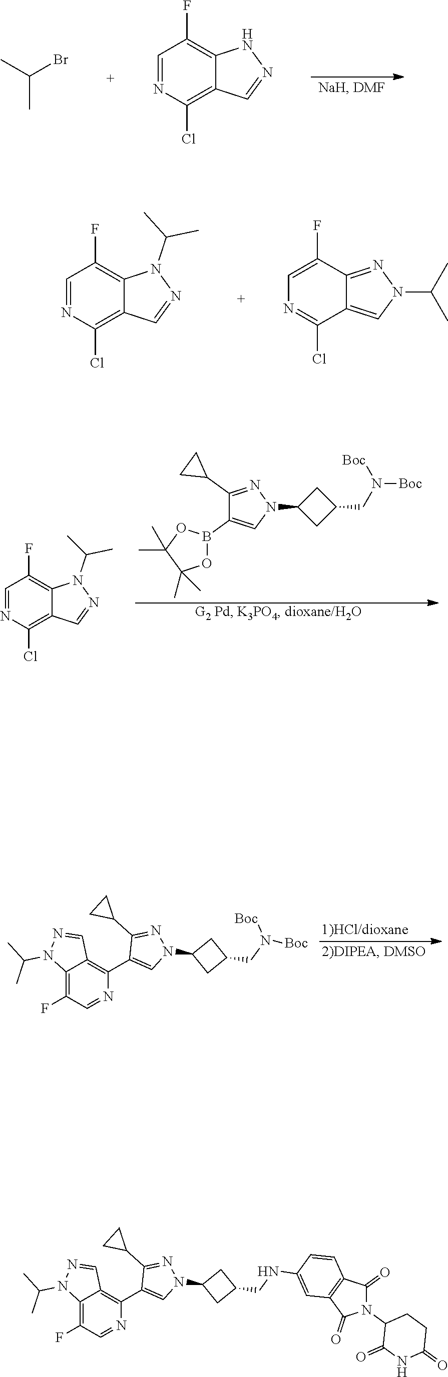

### Step 1. Synthesis of 4-chloro-7-fluoro-1-isopropyl-1H-pyrazolo[4,3-c]pyridine and 4-chloro-7-fluoro-2-isopropyl-2H-pyrazolo[4,3-c]pyridine

To a solution of 4-chloro-7-fluoro-1H-pyrazolo[4,3-c]pyridine (150 mg, 874.33 μmol) in DMF (4 mL) was added NaH (60% in mineral oil, 85 mg, 2.13 mmol) at 0 [symbol] . The mixture was stirred for 30 min, before 2-bromopropane (215 mg, 1.75 mmol) was added. After the mixture was warmed to rt and stirred overnight, it was quenched with methanol (2 mL). The mixture was purified by reverse phase chromatography to afford the title compounds as light brown solids.

4-Chloro-7-fluoro-1-isopropyl-pyrazolo[4,3-c]pyridine (70 mg, yield: 37%). ¹H NMR (400 MHz, CDCl₃) δ 8.15 (d, J=2.4 Hz, 1H), 7.87 (d, J=3.2 Hz, 1H), 4.85-4.79 (m, 1H), 1.65 (s, 3H), 1.64 (s, 3H).

4-Chloro-7-fluoro-2-isopropyl-pyrazolo[4,3-c]pyridine (25 mg, yield: 13%). ¹H NMR (400 MHz, CDCl₃) δ 8.08 (d, J=2.0 Hz, 1H), 7.94 (d, J=3.6 Hz, 1H), 5.04-4.97 (m, 1H), 1.55 (s, 3H), 1.54 (s, 3H).

### Step 2 to 3. Synthesis of 5-(((trans-3-(3-cyclopropyl-4-(7-fluoro-1-isopropyl-1H-pyrazolo[4,3-c]pyridin-4-yl)-1H-pyrazol-1-yl)cyclobutyl)methyl)amino)-2-(2,6-dioxopiperidin-3-yl)isoindoline-1,3-dione

GS-791 was synthesized following the procedures for steps 6 to 8 of GS-790 (12 mg, yield: 6% over 2 steps) as a yellow solid. MS (ESI) m/z=625.6 [M+H]⁺.

### Example 150. (S)-3-(5-(((trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-800)

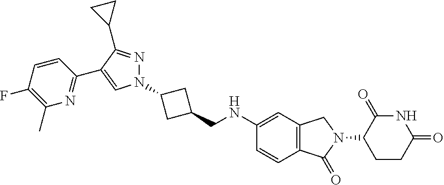

The enantiomers of GS-731 were separated by SFC using a DAICELCHIRALCEL®OJ (250*25 mm 10 μm) as column. The mobile phase was supercritical CO₂ and IPA (+0.1% 7.0 mol/L ammonia in MeOH). And the flow rate was 100 mL/min. The (S)-form of GS-732 was assigned to GS-800. 100% ee; [α]_{D}²⁰=−28.3 [symbol] (c=0.83, MeOH:DCM=1:1);

### Example 151. (R)-3-(5-(((trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-801)

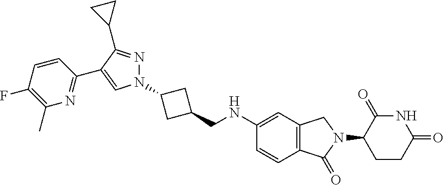 [SMILES: Cc1nc(-c2cn([C@H]3C[C@H](CNc4ccc5c(c4)CN([C@@H]4CCC(=O)NC4=O)C5=O)C3)nc2C2CC2)ccc1F]

The enantiomers of GS-731 were separated by SFC using a DAICELCHIRALCEL®OJ (250*25 mm 10 μm) as column. The mobile phase was supercritical CO₂ and IPA (+0.10% 7.0 mol/L ammonia in MeOH). And the flow rate was 100 mL/min. The (R)-form of GS-732 was assigned to GS-801. 91.9% ee; [α]_{D}²⁰=25.6 [symbol] (c=0.86, MeOH:DCM=1:1);

### Example 152. 3-(5-(((Trans-3-(3-cyclopropyl-4-(6-(difluoromethyl)-5-fluoropyridin-2-yl)-1H-pyrazol-1-yl)cyclobutyl)methyl)amino)-1-oxoisoindolin-2-yl)piperidine-2,6-dione (GS-802)

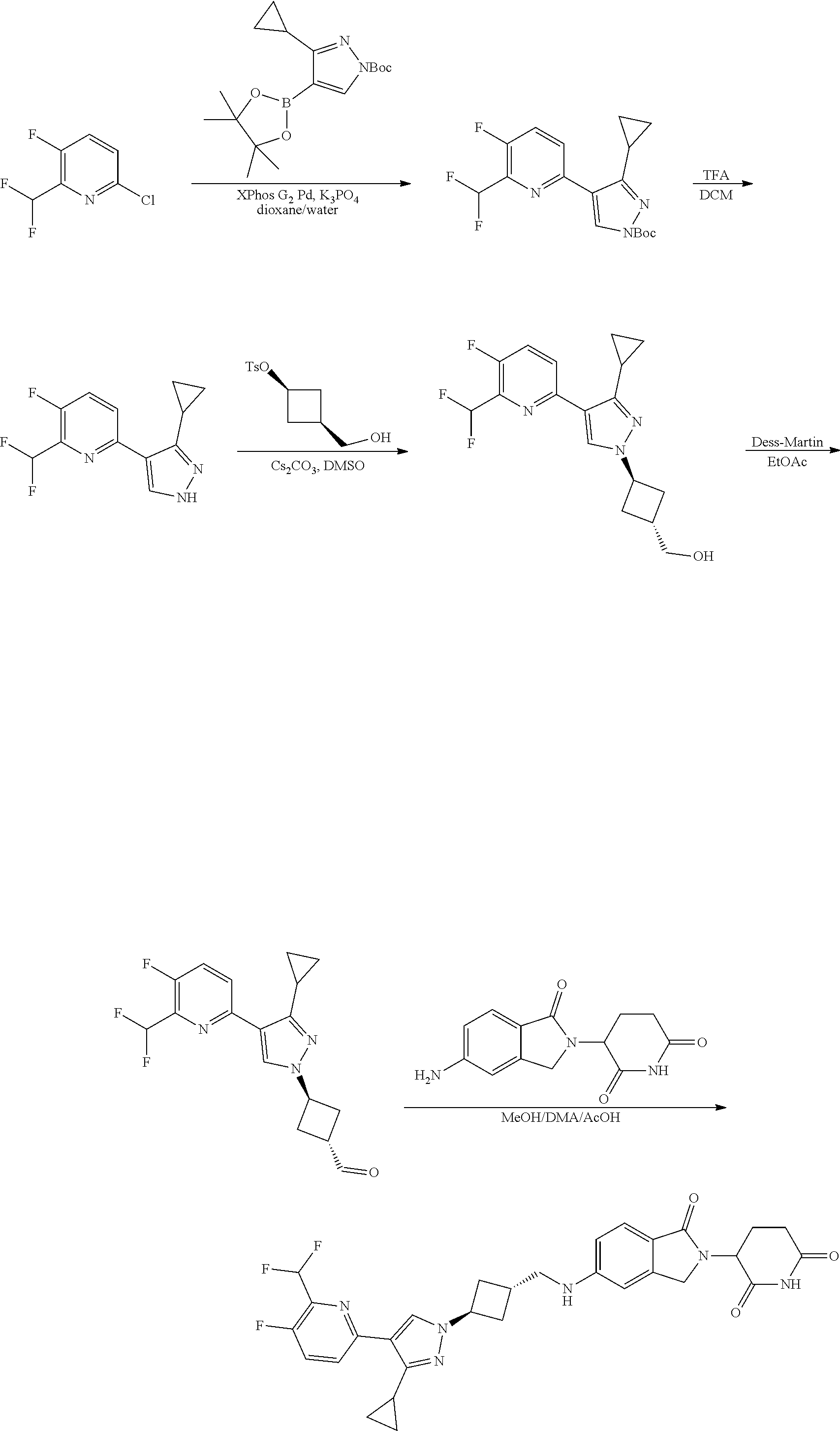

### Step 1. Synthesis of tert-butyl 3-cyclopropyl-4-[6-(difluoromethyl)-5-fluoro-2-pyridyl]pyrazole-1-carboxylate

A mixture of 6-chloro-2-(difluoromethyl)-3-fluoro-pyridine (1.0 g, 5.51 mmol), tert-butyl 3-cyclopropyl-4-(4,4,5,5-tetramethyl-1,3,2-dioxaborolan-2-yl)pyrazole-1-carboxylate (2.5 g, 7.48 mmol), K₃PO₄ (3.82 g, 18.02 mmol), XPhos Pd G2 (150 mg, 5.51 mmol) in dioxane (30 mL) and water (10 mL) was heated at 100 [symbol] under N₂ overnight. The mixture was cooled to rt and concentrated. The residue was purified by silica gel chromatography to provide the title compound (750 mg, yield: 39%) as a brown oil. MS (ESI) m/z=254.5 [M+H-100]⁺.

### Step 2. Synthesis of 6-(3-cyclopropyl-1H-pyrazol-4-yl)-2-(difluoromethyl)-3-fluoro-pyridine

To a solution of tert-butyl 3-cyclopropyl-4-[6-(difluoromethyl)-5-fluoro-2-pyridyl]pyrazole-1-carboxylate (750 mg, 2.12 mmol) in DCM (9 mL) was added TFA (3 mL) at rt. The mixture was stirred at rt for 15 min, before it was concentrated. The residue was evaporated with toluene (30 mL) to provide a solid. The solid was stirred with petroleum ether (25 mL), and then filtered to provide the title compound (450 mg, yield: 84%) as a grey solid. MS (ESI) m/z=254.5 [M+H]⁺.

### Step 3. Synthesis of [3-[3-cyclopropyl-4-[6-(difluoromethyl)-5-fluoro-2-pyridyl]pyrazol-1-yl]cyclobutyl]methanol

A mixture of 6-(3-cyclopropyl-1H-pyrazol-4-yl)-2-(difluoromethyl)-3-fluoro-pyridine (350 mg, 1.38 mmol), [3-(hydroxymethyl)cyclobutyl]4-methylbenzenesulfonate (700 mg, 2.73 mmol) and Cs₂CO₃ (875 mg, 2.69 mmol) in DMSO (5 mL) was heated at 90 [symbol] overnight, before it was cooled to rt, and filtered. The filtrate was purified with reverse phase chromatography to provide the title compound (200 mg, yield: 43%) as a light yellow oil. MS (ESI) m/z=338.26 [M+H]⁺.

### Step 4. Synthesis of 3-[3-cyclopropyl-4-[6-(difluoromethyl)-5-fluoro-2-pyridyl]pyrazol-1-yl]cyclobutanecarbaldehyde

To a solution of [3-[3-cyclopropyl-4-[6-(difluoromethyl)-5-fluoro-2-pyridyl]pyrazol-1-yl]cyclobutyl]methanol (200 mg, 592.88 μmol) in ethyl acetate (5 mL) was added Dess-Martin periodinane (754.39 mg, 1.78 mmol, 550.65 μmol) at rt. The mixture was stirred for 5 h, before it was filtered and concentrated. The residue was purified with silica gel chromatography to provide the title compound (120 mg, yield: 60%) as a colorless oil. MS (ESI) m/z=336.22 [M+H]⁺.

### Step 5. Synthesis of 3-(5-(((trans-3-(3-cyclopropyl-4-(6-(difluoromethyl)-5-fluoropyridin-2-yl)-1H-pyrazol-1-yl)cyclobutyl)methyl)amino)-1-oxoisoindolin-2-yl)piperidine-2,6-dione

A solution of 3-[3-cyclopropyl-4-[6-(difluoromethyl)-5-fluoro-2-pyridyl]pyrazol-1-yl]cyclobutanecarbaldehyde (120 mg, 357.86 μmol), 3-(5-amino-1-oxo-isoindolin-2-yl)piperidine-2,6-dione (85 mg, 327.86 μmol) in methanol (5 mL) and DMA (0.5 mL) was added acetic acid (86 mg, 1.43 mmol) at rt. The mixture was cooled to 0 [symbol] , and then borane-2-picoline complex (96 mg, 897.20 μmol) was added. The mixture was stirred at 0 [symbol] overnight, before it was concentrated and the residue was purified with reverse phase chromatography to provide the title compound (90 mg, yield: 43%) as a white solid. MS (ESI) m/z=579.5 [M+H]⁺.

### Example 153. 3-(5-(((Trans-3-(3-cyclopropyl-4-(5-fluoro-6-(trifluoromethyl)pyridin-2-yl)-1H-pyrazol-1-yl)cyclobutyl)methyl)amino)-1-oxoisoindolin-2-yl)piperidine-2,6-dione (GS-803)

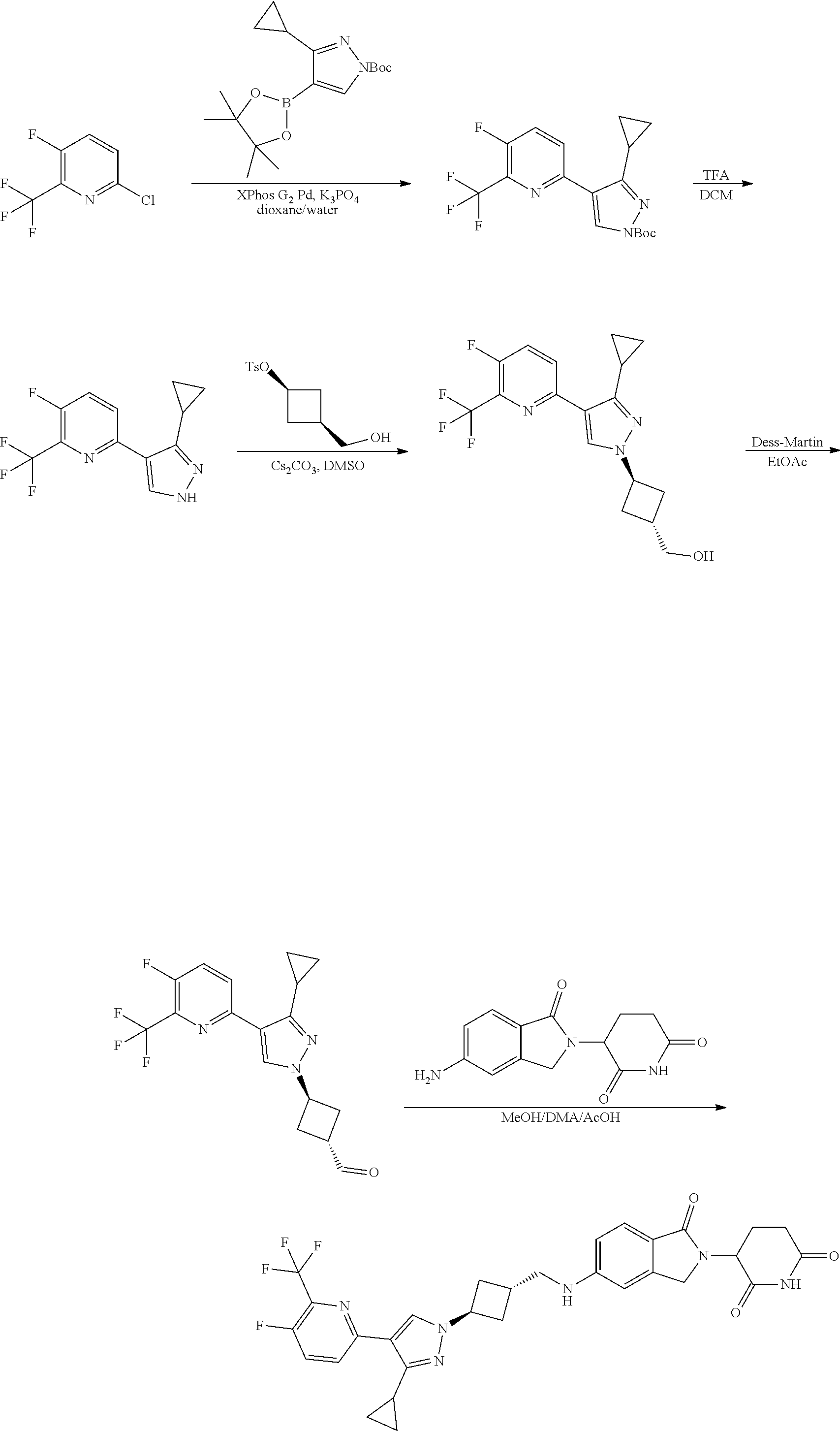

GS-803 was synthesized following the similar procedures as described for GS-802 (48 mg, yield: 2% over 5 steps) as a white solid. MS (ESI) m/z=597.6 [M+H]⁺.

### Example 154. 3-(6-(((Trans-3-(4-(6-amino-5-fluoropyridin-2-yl)-3-cyclopropyl-1H-pyrazol-1-yl)cyclobutyl)methyl)amino)-1-oxoisoindolin-2-yl)piperidine-2,6-dione (GS-804)

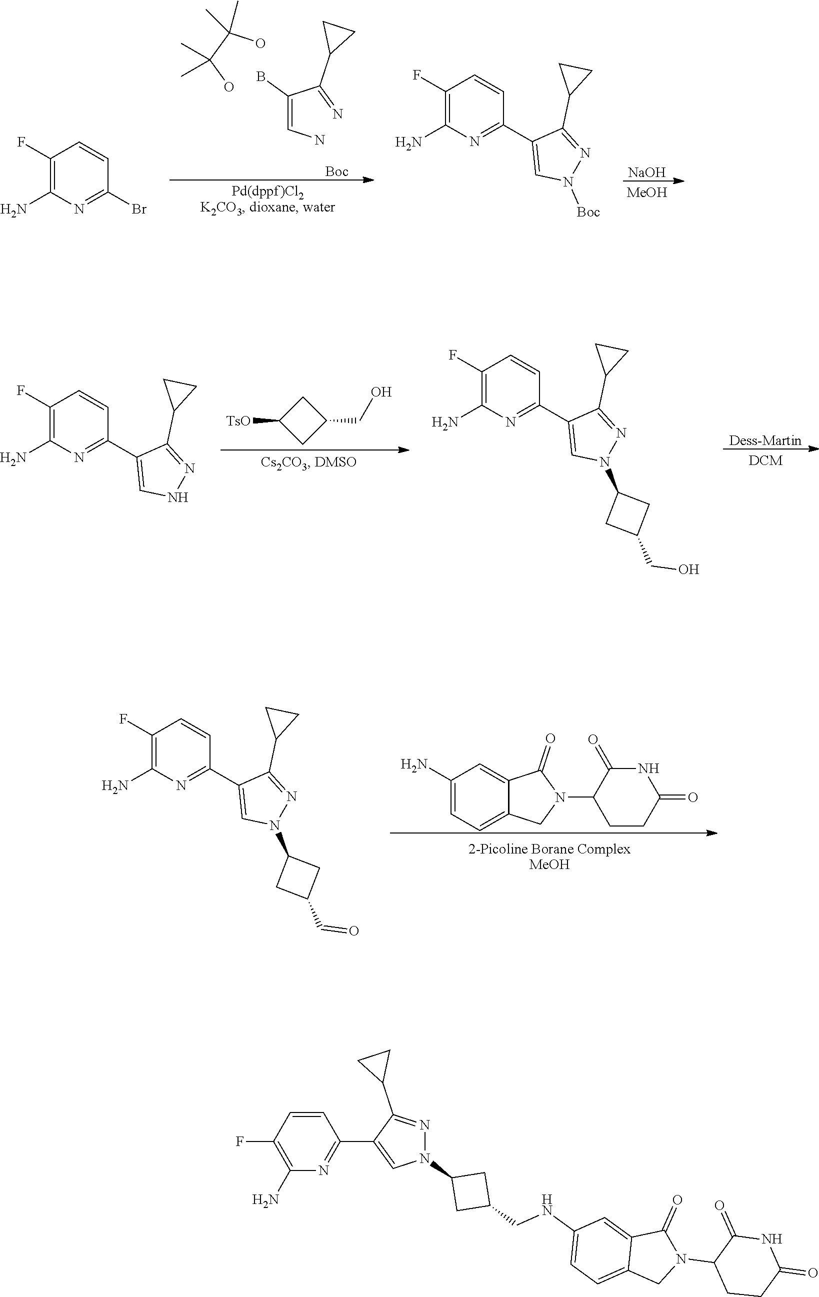

GS-804 was synthesized following the similar procedures as described for GS-788 (18 mg, yield: 1% over 5 steps) as a white solid. MS (ESI) m/z=544.6 [M+H]⁺.

### Example 155. 3-(5-(((Trans-3-(4-(3-chloro-6-methylpyridin-2-yl)-3-cyclopropyl-1H-pyrazol-1-yl)cyclobutyl)methyl)amino)-1-oxoisoindolin-2-yl)piperidine-2,6-dione (GS-805)

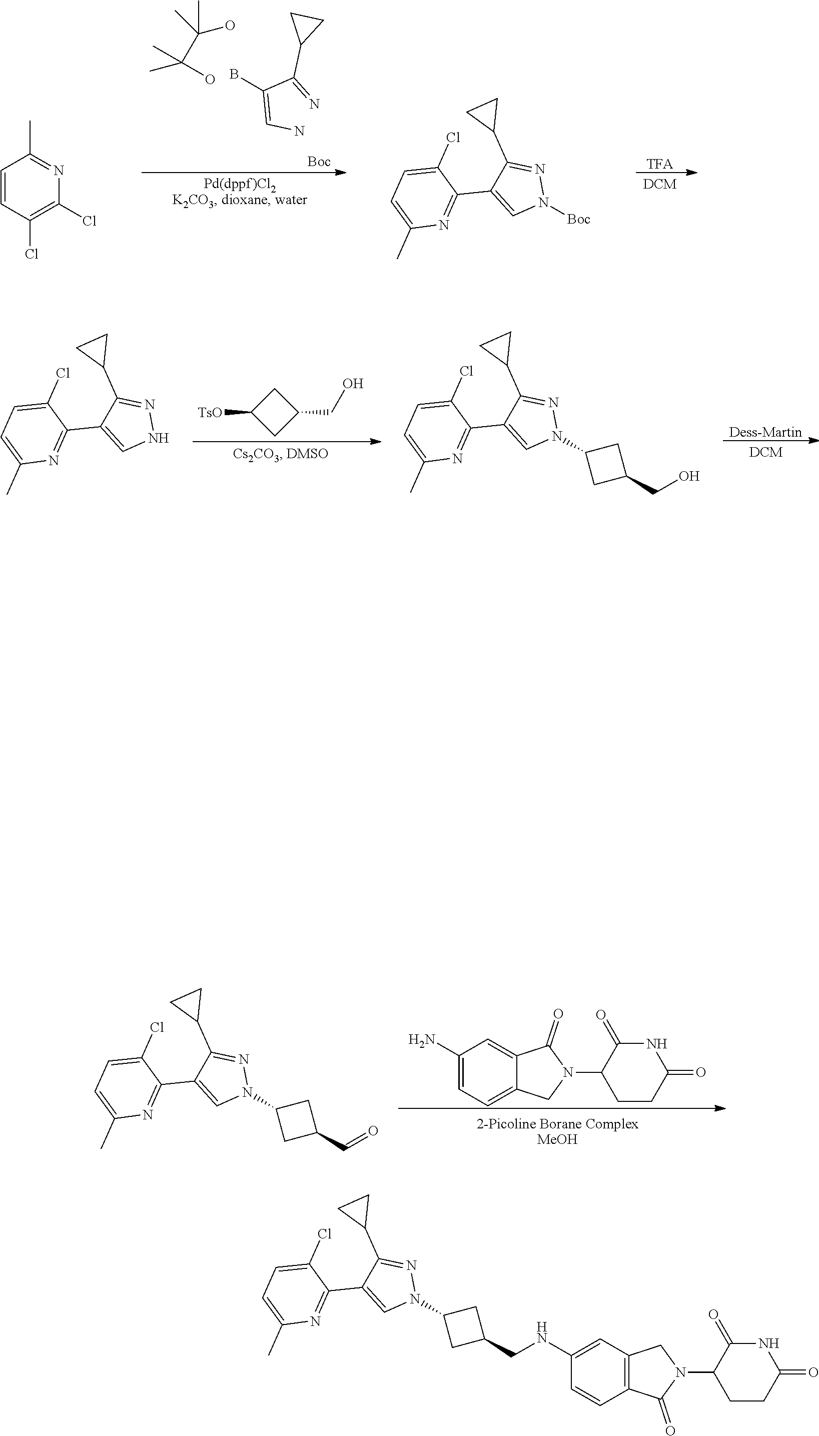

### Step 1. Synthesis of 4-((tert-butyldimethylsilyl)oxy)cyclohexan-1-ol

The title compound was synthesized following the similar procedure as described for step 1 of GS-788 (2.0 g, yield: 49%) as a crude. MS (ESI) m/z 334.5 [M+H]⁺.

### Step 2 to 5. Synthesis of tert-butyl 4-(3-chloro-6-methylpyridin-2-yl)-3-cyclopropyl-1H-pyrazole-1-carboxylate

GS-805 was synthesized following the similar procedures as described for step 2 to 5 of GS-802 (20 mg, yield: 1% over 4 steps) as a white solid. MS (ESI) m/z=559.8 [M+H]⁺.

### Example 156. 3-(5-(((Trans-3-(5-cyclopropyl-4-(6-(dimethylamino)-5-fluoropyridin-2-yl)-1H-pyrazol-1-yl)cyclobutyl)methyl)amino)-1-oxoisoindolin-2-yl)piperidine-2,6-dione (GS-806)

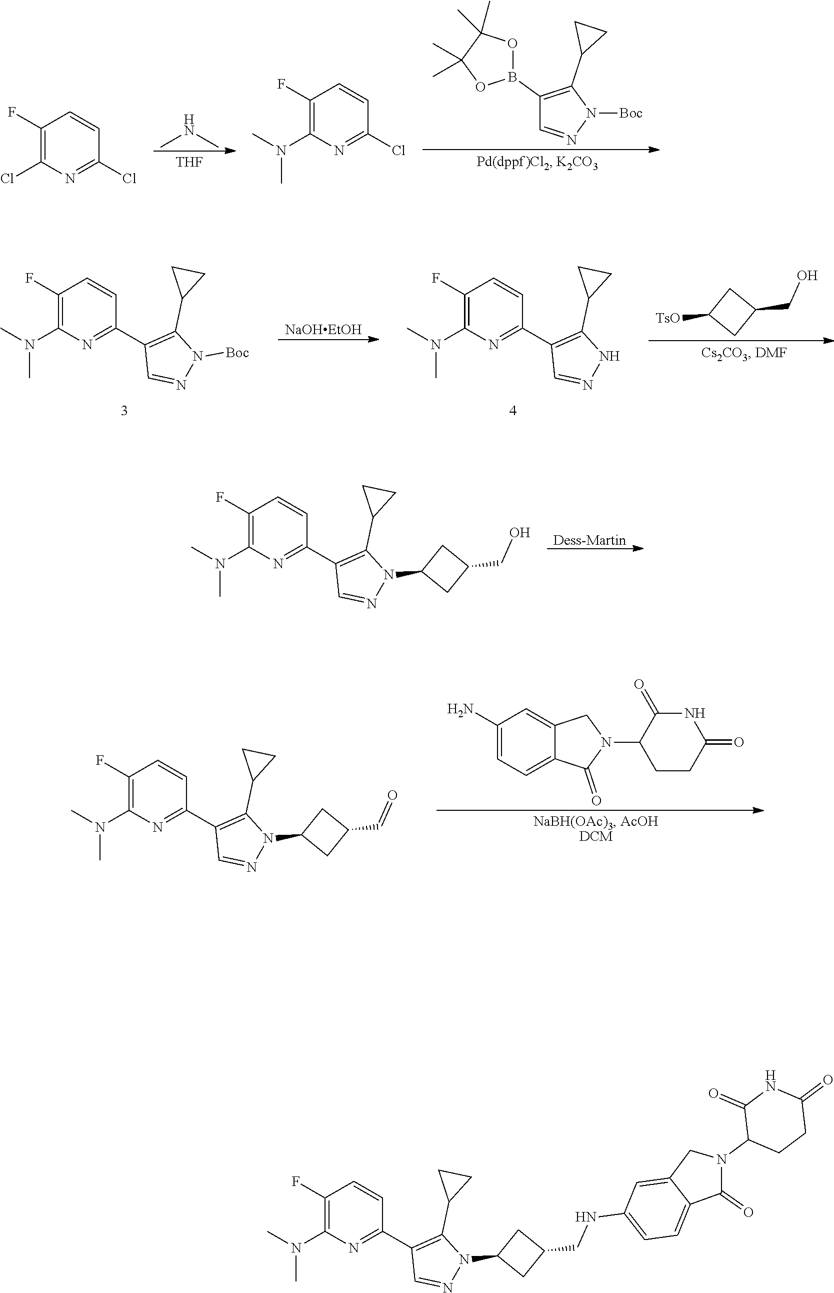

### Step 1. Synthesis of 6-chloro-3-fluoro-N,N-dimethylpyridin-2-amine

To a solution of 2,6-dichloro-3-fluoro-pyridine (500 mg, 3.01 mmol) in THF (10 mL) was added dimethyl amine (136 mg, 3.01 mmol). The mixture was stirred at 80 [symbol] for 16 h, before it was diluted with EtOAc (3 mL). The obtained solution was washed with H₂O and brine. The organic layer was concentrated, and the residue was purified by reverse phase chromatography to provide the title compound (302.54 mg, yield: 58%) as a white solid. MS (ESI) m/z=175.3 [M+H]⁺.

### Step 2 to 6. Synthesis of 3-(5-(((trans-3-(5-cyclopropyl-4-(6-(dimethylamino)-5-fluoropyridin-2-yl)-1-pyrazol-1-yl)cyclobutyl)methyl)amino)-1-oxoisoindolin-2-yl)piperidine-2,6-dione

GS-806 was synthesized following the similar procedures as described for step 1 to 5 of GS-788 (28.7 mg, yield: 4% over 5 steps) as a white solid. MS (ESI) m/z=597.6 [M+H]⁺.

### Example 157. 3-(5-(((Trans-3-(3-cyclopropyl-4-(3-fluoro-6-methylpyridin-2-yl)-1H-pyrazol-1-yl)cyclobutyl)methyl)amino)-1-oxoisoindolin-2-yl)piperidine-2,6-dione (GS-807)

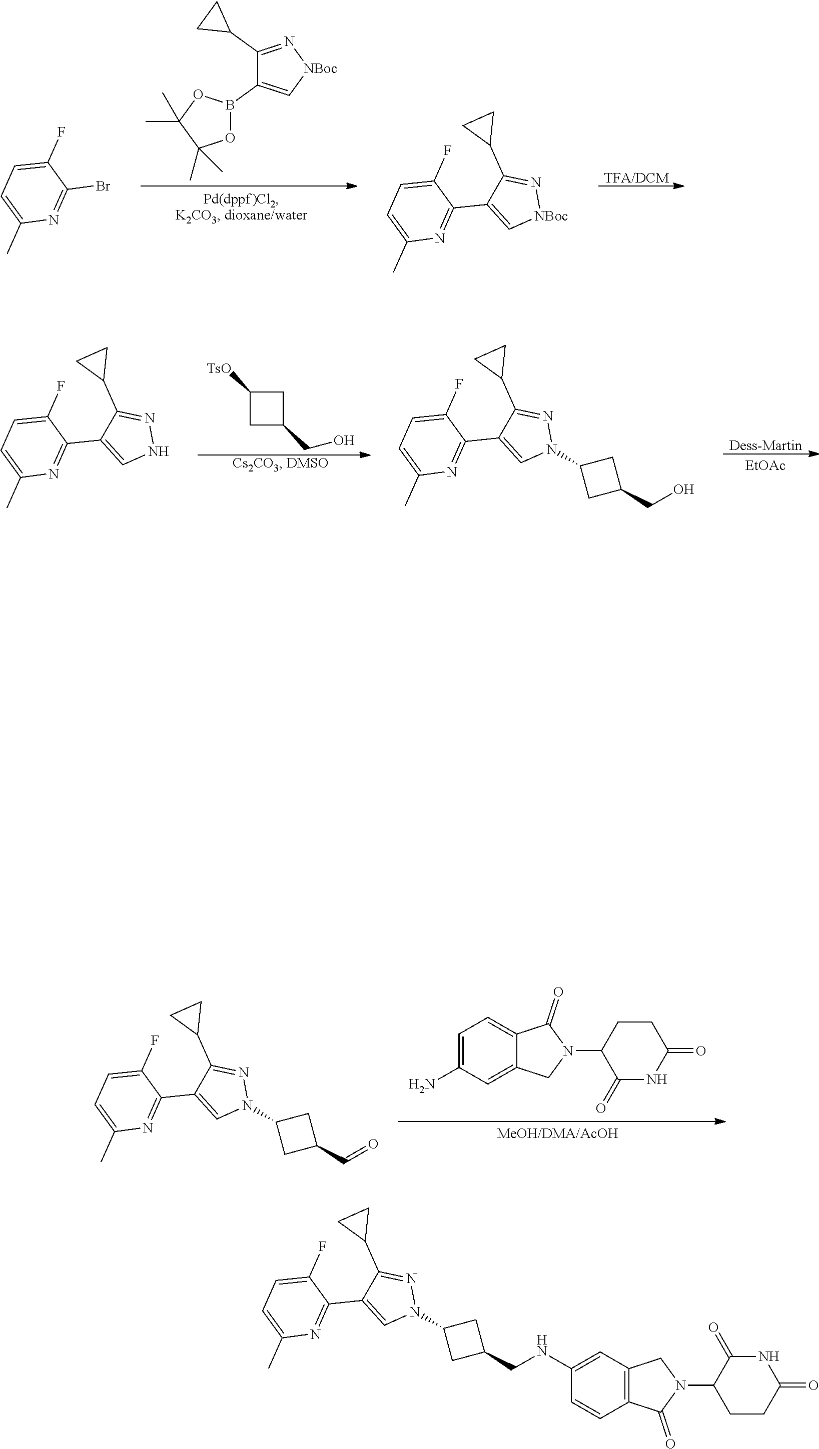

### Step 1. Synthesis of 4-((tert-butyldimethylsilyl)oxy)cyclohexan-1-ol

The title compound was synthesized following the similar procedure as described for step 1 of GS-788 (707 mg, yield: 42%) as a crude. MS (ESI) m/z=318.7 [M+H]⁺.

### Step 2 to 5. Synthesis of 3-(5-(((trans-3-(3-cyclopropyl-4-(3-fluoro-6-methylpyridin-2-yl)-1H-pyrazol-1-yl)cyclobutyl)methyl)amino)-1-oxoisoindolin-2-yl)piperidine-2,6-dione

GS-807 was synthesized following the similar procedures as described for step 2 to 5 of GS-802 (32 mg, yield: 8% over 4 steps) as a yellow solid. MS (ESI) m/z=543.1 [M+H]⁺.

### Example 158. 3-(5-(((Trans-3-(3-cyclopropyl-4-(5-fluoro-6-(methylamino)pyridin-2-yl)-1H-pyrazol-1-yl)cyclobutyl)methyl)amino)-1-oxoisoindolin-2-yl)piperidine-2,6-dione (GS-808)

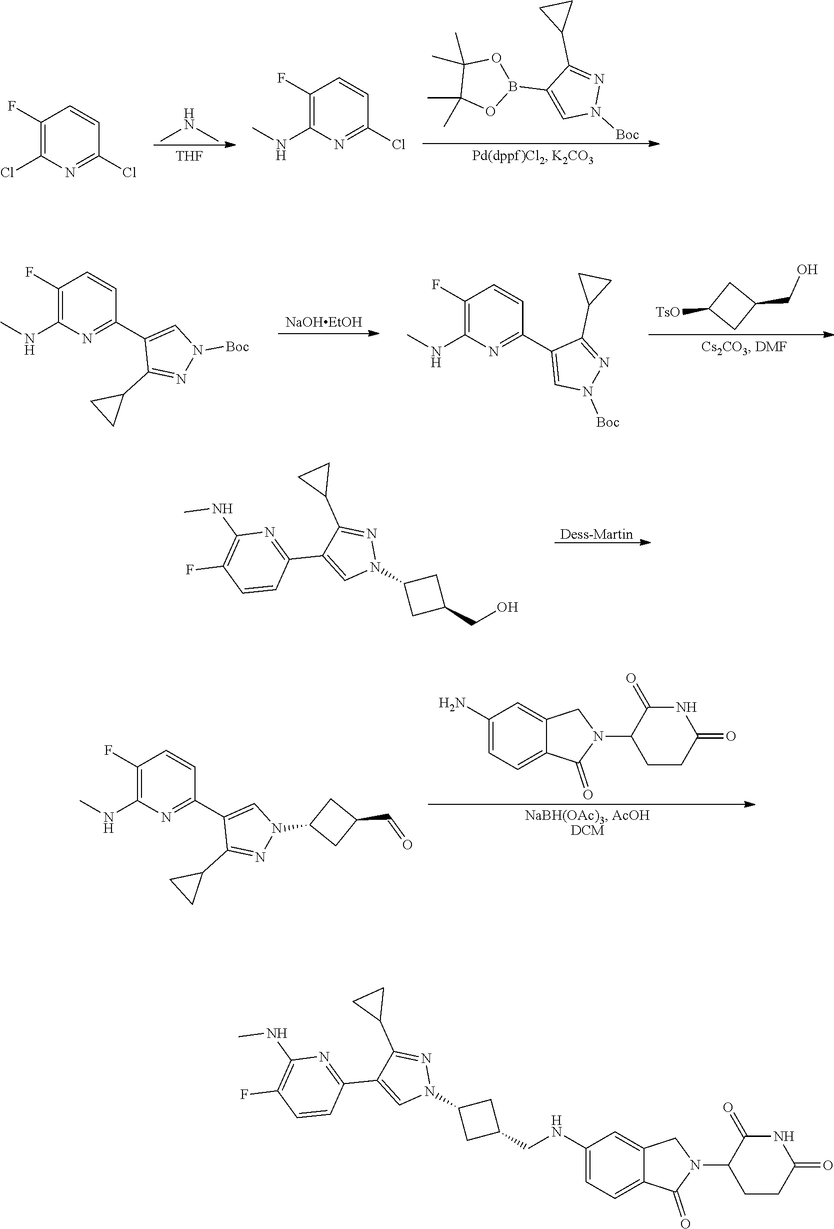

### Step 1. Synthesis of 6-chloro-3-fluoro-N-methylpyridin-2-amine

To a solution of 2,6-dichloro-3-fluoro-pyridine (1.5 g, 9.04 mmol) in THE (10 mL) was added methyl amine (280.7 mg, 9.04 mmol). The mixture was stirred at 80 [symbol] for 16 h, before it was diluted with EtOAc (3 mL) and washed with H₂O and brine. The organic layer was concentrated. The residue was purified by reverse phase chromatography to provide the title compound (510.32 mg, yield: 35%) as a white solid. MS (ESI) m/z=161.3 [M+H]⁺.

### Step 2 to 6. Synthesis of 3-(5-(((trans-3-(3-cyclopropyl-4-(5-fluoro-6-(methylamino)pyridin-2-yl)-1H-pyrazol-1-yl)cyclobutyl)methyl)amino)-1-oxoisoindolin-2-yl)piperidine-2,6-dione

GS-808 was synthesized following the similar procedures as described for step 1 to 5 of GS-788 (22.4 mg, yield: 4% over 5 steps) as a white solid. MS (ESI) m/z=558.7 [M+H]⁺.

### Example 159. 3-(5-(((Trans-3-(3-cyclopropyl-4-(5-fluoro-6-(methylamino)pyridin-2-yl)-1H-pyrazol-1-yl)cyclobutyl)methyl)amino)-1-oxoisoindolin-2-yl)piperidine-2,6-dione (GS-809)

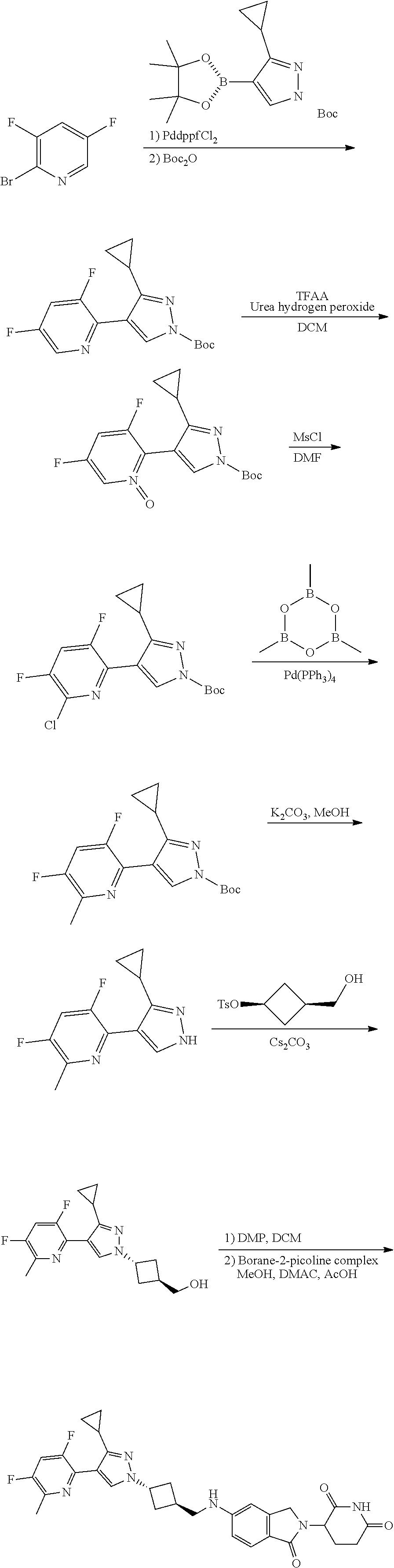

### Step 1. Synthesis of tert-butyl 3-cyclopropyl-4-(3,5-difluoro-2-pyridyl)pyrazole-1-carboxylate

To a solution of 2-bromo-3,5-difluoro-pyridine (4 g, 20.62 mmol) and tert-butyl 3-cyclopropyl-4-(4,4,5,5-tetramethyl-1,3,2-dioxaborolan-2-yl)pyrazole-1-carboxylate (10.34 g, 30.93 mmol) in dioxane (20 mL) and water (5 mL) was added K₂CO₃ (8.54 g, 61.86 mmol) and Pd(dppf)Cl₂ (1.51 g, 2.06 mmol) at rt. The mixture was warmed to 90 [symbol] for 3 h, before it was cooled to rt. Boc₂O (13.55 g, 61.86 mmol) was added. The reaction mixture was stirred at rt for 0.5 h, concentrated and purified by silica gel chromatography (Petroleum ether:EtOAc=20:1 to 10:1) to provide the title compound (5.4 g, yield: 82%) as a white solid. MS (ESI) m/z=222.5 [M-Boc+H]⁺.

### Step 2. Synthesis of tert-butyl 3-cyclopropyl-4-(3,5-difluoro-1-oxido-pyridin-1-ium-2-yl)pyrazole-1-carboxylate

To a solution of tert-butyl 3-cyclopropyl-4-(3,5-difluoro-2-pyridyl)pyrazole-1-carboxylate (5.0 g, 15.56 mmol) and urea hydrogen peroxide (2.93 g, 31.12 mmol) in DCM (100 mL) was added TFAA (6.54 g, 31.12 mmol) dropwise. The reaction mixture was stirred at rt for 16 h before it was quenched with aq. NaHCO₃ (50 mL), and then washed with aq. NaHSO₃ solution (100 mL). The organic phase was concentrated and purified by silica gel chromatography (petroleum ether:EtOAc=5:1 to 1:1) to provide the title compound (3.2 g, yield: 61%). MS (ESI) m/z=238.5 [M-Boc+H]⁺.

### Step 3. Synthesis of tert-butyl 4-(6-chloro-3,5-difluoro-2-pyridyl)-3-cyclopropyl-pyrazole-1-carboxylate

To a solution of tert-butyl 3-cyclopropyl-4-(3,5-difluoro-1-oxido-pyridin-1-ium-2-yl)pyrazole-1-carboxylate (3.2 g, 9.49 mmol) in DMF (50 mL) was added MsCl (5.43 g, 47.43 mmol) at rt. The mixture was warmed to 90 [symbol] for 3 h, before it was cooled to rt, and quenched with brine (40 mL). The obtained mixture was extracted with EtOAc (40 mL×2). The combined organic layer was washed with brine (20 mL×3) and concentrated. The residue was purified by silica gel chromatography (petroleum ether:EtOAc=10:1 to 2:1) to provide the title compound (1.3 g, yield: 39%) as a yellow solid. MS (ESI) m/z=256.4 [M-Boc+H]⁺.

### Step 4. Synthesis of tert-butyl 3-cyclopropyl-4-(3,5-difluoro-6-methyl-2-pyridyl)pyrazole-1-carboxylate

To a solution of tert-butyl 4-(6-chloro-3,5-difluoro-2-pyridyl)-3-cyclopropyl-pyrazole-1-carboxylate (1.3 g, 3.65 mmol) and 2,4,6-trimethyl-1,3,5,2,4,6-trioxatriborinane (917.43 mg, 7.31 mmol) in dioxane (50 mL) was added Pd(dppf)Cl₂ (267.11 mg, 365.41 μmol) and K₂CO₃ (1.51 g, 10.96 mmol) at rt under N₂. The reaction mixture was stirred at 110 [symbol] for 3 days, before it was concentrated and purified by silica gel chromatography (petroleum ether:EtOAc=3:1 to 1:1) to provide the title compound (1.2 g, yield: 98.0%) as a white solid. MS (ESI) m/z=236.4 [M-Boc+H]⁺.

### Step 5. Synthesis of 2-(3-cyclopropyl-1H-pyrazol-4-yl)-3,5-difluoro-6-methyl-pyridine

To a solution of tert-butyl 3-cyclopropyl-4-(3,5-difluoro-6-methyl-2-pyridyl)pyrazole-1-carboxylate (1.2 g, 3.58 mmol) in methanol (50 mL) was added K₂CO₃ (1.98 g, 14.31 mmol) at rt. The mixture was warmed to 68 [symbol] for 1 h, before it was concentrated. The residue was purified by silica gel chromatography (petroleum ether:EtOAc=1:1 to 0:1) to provide the title compound (800 mg, yield: 95.0%) as a white solid. MS(ESI) m/z=236.4[M+H]⁺.

### Step 6. Synthesis of (trans-3-(3-cyclopropyl-4-(3,5-difluoro-6-methylpyridin-2-yl)-1H-pyrazol-1-yl)cyclobutyl)methanol

To a solution of 2-(3-cyclopropyl-1H-pyrazol-4-yl)-3,5-difluoro-6-methyl-pyridine (800 mg, 3.40 mmol) in DMSO (40 mL) was added cis-3-(hydroxymethyl)cyclobutyl 4-methylbenzenesulfonate (1.74 g, 6.80 mmol) and Cs₂CO₃ (2.22 g, 6.80 mmol) at rt. The mixture was warmed to 100 [symbol] for 2 h, before it was concentrated. The residue was purified by reverse phase chromatography (0.05% TFA in water:MeOH=1:0 to 0:1) to provide the crude. The residue was purified by silica gel chromatography (petroleum ether:EtOAc=1:0 to 3:1) to provide the title compound (780 mg, yield: 72%) as a white solid. MS(ESI) m/z=320.6 [M+H]⁺.

### Step 7. Synthesis of 3-(5-(((trans-3-(3-cyclopropyl-4-(3,5-difluoro-6-methylpyridin-2-yl)-1H-pyrazol-1-yl)cyclobutyl)methyl)amino)-1-oxoisoindolin-2-yl)piperidine-2,6-dione

To a solution of (trans-3-(3-cyclopropyl-4-(3,5-difluoro-6-methylpyridin-2-yl)-1H-pyrazol-1-yl)cyclobutyl)methanol (200 mg, 626.28 μmol) in DCM (10 mL) was added Dess-Martin periodinane (531.08 mg, 1.25 mmol) at rt. The reaction mixture was stirred at rt for 2 h, before it was quenched with aq. NaHCO₃ solution (10 mL). The DCM layer was washed with brine (10 mL), dried over Na₂SO₄, filtrated and concentrated. The obtained intermediate was dissolved in MeOH (10 mL) and DMAc (5 mL). AcOH (150.31 mg, 2.51 mmol), 3-(5-amino-1-oxo-isoindolin-2-yl)piperidine-2,6-dione (162.37 mg, 626.28 μmol) and borane-2-picoline complex (137.77 mg, 1.25 mmol) were added at 0 [symbol] . The reaction mixture was stirred at 0 [symbol] for 2 h, and then stirred at rt for 16 h. The residue was purified by reverse phase chromatography (0.05% TFA in water:MeOH=1:0 to 1:2) and silica gel chromatography (petroleum ether:EtOAc=1:1 to 0:1) to provide the title compound (175 mg, yield: 49.85% yield) as a white solid. MS(ESI) m/z=561.6 [M+H]⁺.

### Example 160. 3-(5-(((Trans-3-(4-(6-aminopyridin-2-yl)-3-cyclopropyl-1H-pyrazol-1-yl)cyclobutyl)methyl)amino)-1-oxoisoindolin-2-yl)piperidine-2,6-dione (GS-810)

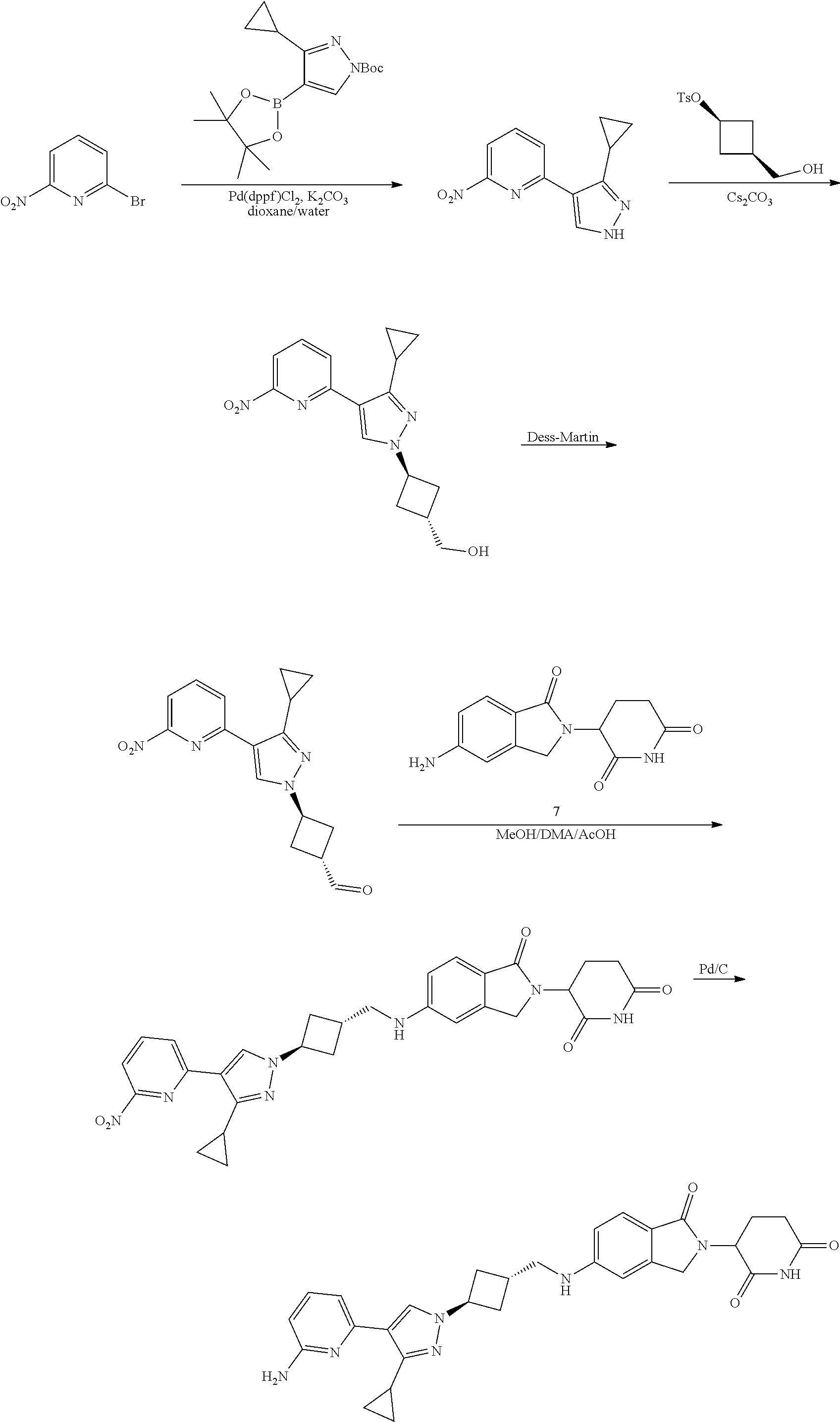

### Step 1. Synthesis of 2-(3-cyclopropyl-1H-pyrazol-4-yl)-6-nitropyridine

A mixture of 2-bromo-6-nitro-pyridine (500 mg, 2.46 mmol), tert-butyl 3-cyclopropyl-4-(4,4,5,5-tetramethyl-1,3,2-dioxaborolan-2-yl)pyrazole-1-carboxylate (1.23 g, 3.69 mmol), Pd(dppf)Cl₂ (60 mg, 2.46 mmol) and K₂CO₃ (680 mg, 4.93 mmol) in dioxane (50 mL) and water (15 mL) was heated at 90 [symbol] overnight under N₂, before it was poured to water (50 mL). The obtained mixture was extracted with ethyl acetate (50 mL) twice. The combined organic layer was washed with brine (50 mL), dried and concentrated. The residue was purified with silica gel chromatography to provide the title compound (170 mg, yield: 30%) as a brown solid. MS (ESI) m/z=231.4 [M+H]⁺.

### Step 2 to 5. Synthesis of 3-(5-(((trans-3-(3-cyclopropyl-4-(6-nitropyridin-2-yl)-1H-pyrazol-1-yl)cyclobutyl)methyl)amino)-1-oxoisoindolin-2-yl)piperidine-2,6-dione

The title compound was synthesized following the similar procedure as described for step 3 to 5 of GS-788 (15 mg, yield: 4% over 3 steps) as a light yellow solid. MS (ESI) m/z=556.6 [M+H]⁺.

### Step 6. Synthesis of 3-(5-(((trans-3-(4-(6-aminopyridin-2-yl)-3-cyclopropyl-1H-pyrazol-1-yl)cyclobutyl)methyl)amino)-1-oxoisoindolin-2-yl)piperidine-2,6-dione

To a solution of 3-(5-(((trans-3-(3-cyclopropyl-4-(6-nitropyridin-2-yl)-1H-pyrazol-1-yl)cyclobutyl)methyl)amino)-1-oxoisoindolin-2-yl)piperidine-2,6-dione (15 mg, 27.00 μmol) in methanol (6 mL) and DMAc (2 mL) was added Pd/C (6 mg, 49.40 μmol) at rt. The reaction mixture was stirred under H₂ at 0 [symbol] for 2 h, before it was filtered and concentrated. The residue was purified with prep-HPLC to provide the title compound (3 mg, yield: 21%) as a white solid. MS (ESI) m/z=526.6 [M+H]⁺.

### Example 161. 3-(5-(((Trans-3-(3-cyclopropyl-4-(6-(methylamino)pyridin-2-yl)-1H-pyrazol-1-yl)cyclobutyl)methyl)amino)-1-oxoisoindolin-2-yl)piperidine-2,6-dione (GS-811)

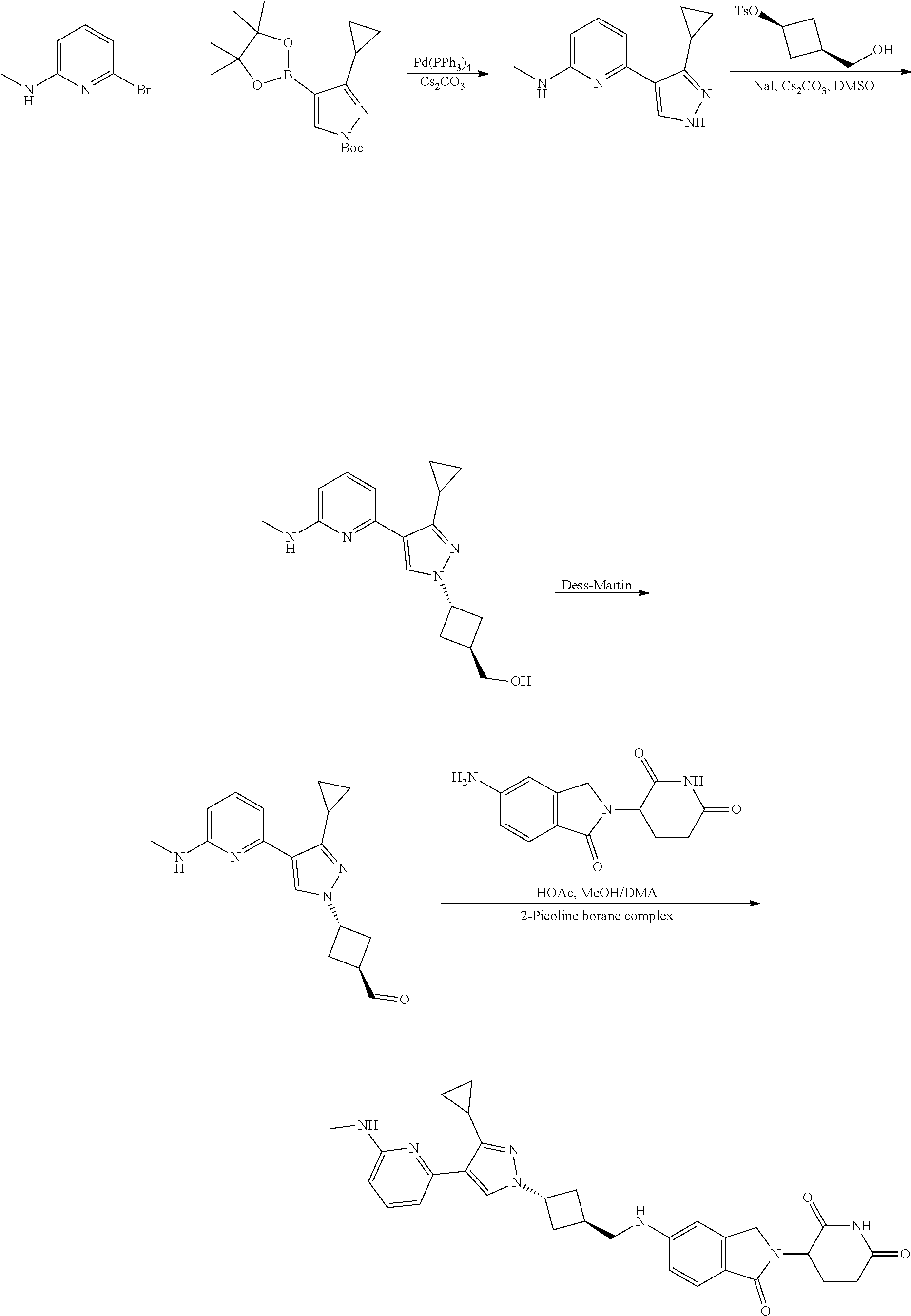

### Step 1. Synthesis of 6-(3-cyclopropyl-1H-pyrazol-4-yl)-N-methylpyridin-2-amine

To a solution of 6-bromo-N-methyl-pyridin-2-amine (500 mg, 2.67 mmol), tert-butyl 3-cyclopropyl-4-(4,4,5,5-tetramethyl-1,3,2-dioxaborolan-2-yl)pyrazole-1-carboxylate (1.34 g, 4.01 mmol), in 1,4-dioxane (8 mL) and H₂O (1.5 mL) were added Pd(PPh₃)₄ (308.91 mg, 267.33 μmol) and Cs₂CO₃ (2.61 g, 8.02 mmol). The reaction mixture was stirred at 100 [symbol] for 16 h, before it was concentrated and poured into ice/water. The mixture was extracted with EtOAc (20 mL×3). The combined organic layer was washed with brine, dried over Na₂SO₄, filtered and concentrated. The residue was purified by reverse phase chromatography. The desired fractions were combined and aq. sat. NaHCO₃ solution was added until pH=6˜7. The mixture was then extracted with EtOAc (30 mL×2). The organic layer was dried over Na₂SO₄ and concentrated to provide the desired product (290 mg, yield: 51%) as an oil.

### Step 2 to 4. Synthesis of 3-(5-(((trans-3-(3-cyclopropyl-4-(6-(methylamino)pyridin-2-yl)-1H-pyrazol-1-yl)cyclobutyl)methyl)amino)-1-oxoisoindolin-2-yl)piperidine-2,6-dione

GS-811 was synthesized following the similar procedures as described for step 3 to 5 of GS-788 (12 mg, yield: 2% over 3 steps) as a white solid. MS (ESI) m/z=540.8 [M+H]⁺.

### Example 162. 3-(5-(((Trans-3-(4-(6-aminopyridin-2-yl)-3-cyclopropyl-1H-pyrazol-1-yl)cyclobutyl)methyl)amino)-1-oxoisoindolin-2-yl)piperidine-2,6-dione (GS-812)

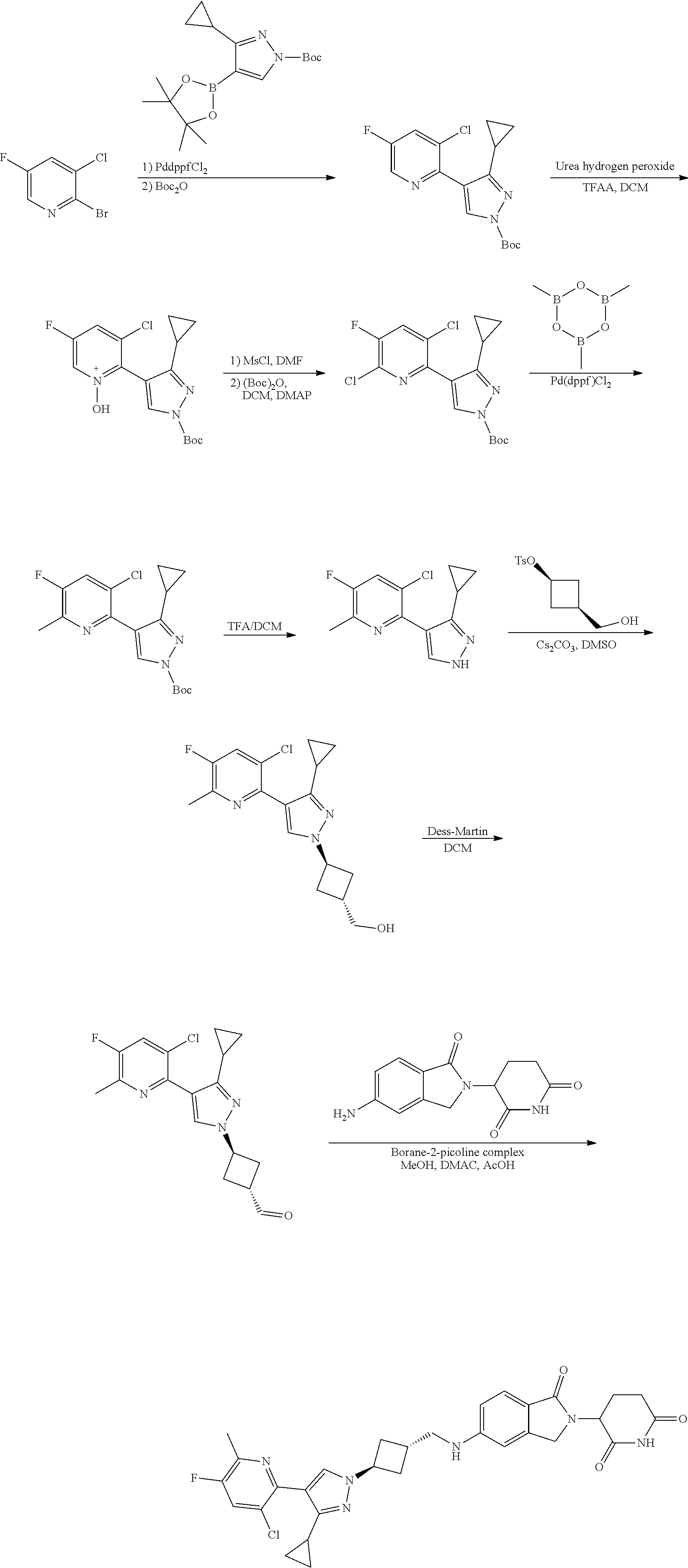

### Step 1 to 2. Synthesis of 2-(1-(tert-butoxycarbonyl)-3-cyclopropyl-1H-pyrazol-4-yl)-3-chloro-5-fluoropyridine 1-oxide

The title compound was synthesized following the similar procedure as described for step 1 to 2 of GS-809 (900 mg, yield: 18% over 2 steps) as a light yellow solid. MS (ESI) m/z=354.4 [M+H]⁺.

### Step 3. Synthesis of tert-butyl 3-cyclopropyl-4-(3,6-dichloro-5-fluoropyridin-2-yl)-1H-pyrazole-1-carboxylate

To a solution of 2-(1-(tert-butoxycarbonyl)-3-cyclopropyl-1H-pyrazol-4-yl)-3-chloro-5-fluoropyridine 1-oxide (900 mg, 2.54 mmol) in DMF (5 mL) was added MsCl (1.46 g, 12.72 mmol) at rt. The reaction mixture was heated at 90 [symbol] for 3 h, before it was diluted with EtOAc (100 mL). The organic phase was washed with K₂CO₃ solution (aq, 100%, 50 mL) and brine (50 mL), dried and concentrated. The crude intermediate was dissolved in DCM (30 mL). DMAP (100 mg, 818.55 μmol) and (Boc)₂O (1.10 g, 5.09 mmol) were added at rt. The mixture was stirred for 1 h, before it was concentrated. The residue was purified by silica gel chromatography (petroleum ether:EtOAc=0˜10%) to provide the title compound (600 mg, yield: 63%) as a light yellow oil. MS (ESI) m/z=372.3 [M+H]⁺.

### Step 4. Synthesis of tert-butyl 4-(3-chloro-5-fluoro-6-methylpyridin-2-yl)-3-cyclopropyl-1H-pyrazole-1-carboxylate

A mixture of tert-butyl 3-cyclopropyl-4-(3,6-dichloro-5-fluoro-2-pyridyl)pyrazole-1-carboxylate (450 mg, 1.21 mmol), 2,4,6-trimethyl-1,3,5,2,4,6-trioxatriborinane (439.37 mg, 3.5 mmol), K₂CO₃ (167.09 mg, 1.21 mmol) and PdCl₂(dppf) (100 mg, 130.93 μmol) in dioxane (10 mL) was heated at 80 [symbol] under N₂ for 3 h, before it was cooled to rt. The mixture was poured into water (50 mL), extracted with ethyl acetate (35 mL×3). The combined organic layers were washed with brine (30 mL), dried and concentrated. The residue was purified with silica gel chromatography to provide the title compound (420 mg, yield: 99%) as a colorless oil. MS (ESI) m/z=352.4 [M+H]⁺.

### Step 5-8. Synthesis of 3-(5-(((trans-3-(4-(6-aminopyridin-2-yl)-3-cyclopropyl-1H-pyrazol-1-yl)cyclobutyl)methyl)amino)-1-oxoisoindolin-2-yl)piperidine-2,6-dione

GS-812 was synthesized following the similar procedures as described for step 2 to 5 of GS-802 (90 mg, yield: 16% over 4 steps) as a white solid. MS (ESI) m/z=577.5 [M+H]⁺.

### Example 163. (S)-3-(5-(((trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-813)

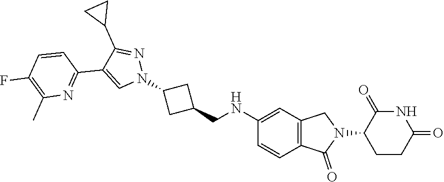 [SMILES: Cc1nc(-c2cn([C@H]3C[C@H](CNc4ccc5c(c4)CN([C@H]4CCC(=O)NC4=O)C5=O)C3)nc2C2CC2)ccc1F]

The enantiomers of GS-807 were separated by SFC using a DAICELCHIRALCEL®OJ (250*25 mm 10 μm) as column. The mobile phase was supercritical CO₂ and IPA. And the flow rate was 100 mL/min. The (S)-form of GS-807 was assigned to GS-813. 94.7% ee; [α]_{D}²⁰=−28.0 [symbol] (c=0.90, MeOH:DCM=1:1);

### Example 164. (R)-3-(5-(((trans-3-(3-cyclopropyl-4-(5-fluoro-6-methylpyridin-2-yl)-1H-pyrazol-1-yl)cyclobutyl)methyl)amino)-1-oxoisoindolin-2-yl)piperidine-2,6-dione (GS-814)

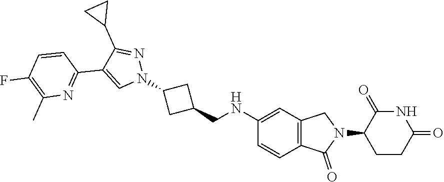 [SMILES: Cc1nc(-c2cn([C@H]3C[C@H](CNc4ccc5c(c4)CN([C@@H]4CCC(=O)NC4=O)C5=O)C3)nc2C2CC2)ccc1F]

The enantiomers of GS-807 were separated by SFC using a DAICELCHIRALCEL®OJ (250*25 mm 10 μm) as column. The mobile phase was supercritical CO₂ and IPA. And the flow rate was 100 mL/min. The (R)-form of GS-807 was assigned to GS-814. 100% ee; [α]_{D}²⁰=25.0 [symbol] (c=0.89, MeOH:DCM=1:1).

### Materials and Methods

### Example 165. In Vitro Testing

Cell Culture: RS4;11, MV4;11, MOLM-13 and other cells were cultured at 37° C. with 5% CO₂ in RPMI 1640 or DMEM Medium supplemented with 10% fetal bovine serum. Cells were authenticated using the short tandem repeat (STR) assays. Mycoplasma test results were negative.

Antibodies and reagents: Rabbit anti-FLT3 antibody (3462S) was purchased from Cell Signaling Technology. Rabbit anti-GSPT1 antibody (ab126090) was purchased from Abeam. HRP-conjugated anti-α-tubulin antibody was produced in house. Media and other cell culture reagents were purchased from Thermo Fisher. The CellTiter-Glo Luminescent Assay kit was purchased from Promega.

Immunoblotting: Cultured cells were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the accompanied software provided by Bio-Rad.

Cell viability assays: Cells were seeded at a density of 5000 cells per well in 96-well assay plates and treated with test compounds following a 8-point or 12-point 3-fold serial dilution. Three days later, cell viability was determined using the CellTiter-Glo assay kit according to the manufacturer's instructions. The dose-response curves were determined and IC₅₀ values were calculated using the GraphPad Prism software following a nonlinear regression (least squares fit) method.

The IC₅₀ values (nM) of each compound were determined in MV4;11 and MOLM-13 cells with or without gilteritinib (10 nM for MV4;11; and 20 nM for MOML-13) as described in the Materials and Methods subsection and calculated using the GraphPad Prism 5.0 software.

The cell viability inhibition results of selected compounds are set forth in Table 2. The compounds were tested in both MV4;11 cells and in MOLM-13 cells, either alone or in combination with gilteritinib.

TABLE 2
Exemplary results in MV4; 11 and MOLM-13 cells.

| | MV4; 11 | | MOLM-13 | |
|---|---|---|---|---|
| | | IC₅₀ (nM) + 10 | | IC₅₀ (nM) + 10 nM |
| Cpd. | IC₅₀ (nM) | nM Gil | IC₅₀ (nM) | Gil |
| GS-640 | D | C | D | C |
| GS-641 | A | A | A | A |
| GS-642 | B | A | B | A |
| GS-643 | D | D | D | D |
| GS-644 | B | A | B | A |
| GS-645 | D | D | D | C |
| GS-646 | B | B | C | B |
| GS-647 | D | C | D | B |
| GS-648 | D | D | D | C |
| GS-649 | A | A | B | A |
| GS-650 | B | B | C | B |
| GS-651 | A | A | B | A |
| GS-652 | D | B | D | C |
| GS-653 | C | B | D | B |
| GS-654 | D | B | C | C |
| GS-655 | C | B | D | C |
| GS-656 | B | B | C | B |
| GS-657 | B | A | B | A |
| GS-658 | D | C | D | C |
| GS-659 | A | A | A | A |
| GS-660 | A | A | A | A |
| GS-661 | B | A | B | A |
| GS-662 | D | D | D | D |
| GS-663 | D | D | D | C |
| GS-664 | D | C | D | C |
| GS-665 | D | D | D | D |
| GS-666 | D | D | D | D |
| GS-667 | B | A | B | B |
| GS-668 | A | A | A | A |
| GS-669 | C | C | D | C |
| GS-670 | B | B | C | B |
| GS-671 | D | D | D | C |
| GS-672 | D | D | D | C |
| GS-673 | D | C | D | C |
| GS-674 | D | D | D | D |
| GS-675 | D | C | D | C |
| GS-676 | A | A | A | A |
| GS-677 | A | A | A | A |
| GS-678 | A | A | A | A |
| GS-679 | D | C | D | C |
| GS-680 | A | A | B | A |
| GS-681 | D | C | D | B |
| GS-682 | B | A | C | B |
| GS-683 | D | D | D | D |
| GS-684 | B | B | C | B |
| GS-685 | D | C | D | C |
| GS-686 | D | C | D | C |
| GS-687 | D | C | D | C |
| GS-688 | D | D | D | C |
| GS-689 | B | B | C | B |
| GS-690 | D | D | D | D |
| GS-691 | D | D | D | D |
| GS-693 | D | C | D | C |
| GS-694 | D | C | D | C |
| GS-695 | B | A | B | A |
| GS-696 | C | B | D | B |
| GS-697 | A | A | A | A |
| GS-699 | B | B | C | B |
| GS-700 | C | B | C | B |
| GS-701 | B | B | C | B |
| GS-702 | A | A | A | A |
| GS-703 | B | B | C | B |
| GS-704 | B | A | B | A |
| GS-705 | D | C | C | C |
| GS-706 | B | A | B | A |
| GS-707 | B | A | B | B |
| GS-708 | D | D | D | C |
| GS-709 | D | D | D | D |
| GS-710 | B | A | B | A |
| GS-711 | D | D | D | D |
| GS-712 | A | A | A | A |
| GS-713 | B | B | C | B |
| GS-714 | B | B | C | B |
| GS-715 | D | C | D | D |
| GS-716 | D | D | D | D |
| GS-717 | A | A | B | A |
| GS-718 | D | D | D | D |
| GS-719 | B | A | B | A |
| GS-720 | B | B | C | B |
| GS-721 | A | A | B | A |
| GS-722 | B | A | B | A |
| GS-723 | C | B | C | B |
| GS-724 | A | A | B | A |
| GS-725 | C | B | D | C |
| GS-726 | D | C | D | C |
| GS-727 | D | C | D | C |
| GS-728 | D | D | D | B |
| GS-729 | B | A | B | A |
| GS-730 | B | B | C | B |
| GS-731 | A | A | B | A |
| GS-732 | C | B | D | C |
| GS-733 | A | A | A | A |
| GS-735 | B | A | B | A |
| GS-736 | A | A | B | A |
| GS-737 | B | B | C | B |
| GS-738 | B | B | C | B |
| GS-739 | B | A | B | A |
| GS-740 | D | D | D | C |
| GS-741 | B | A | B | B |
| GS-742 | D | C | D | C |
| GS-743 | A | A | A | A |
| GS-744 | C | B | D | B |
| GS-745 | D | D | D | D |
| GS-746 | C | B | D | B |
| GS-747 | B | A | B | A |
| GS-748 | B | A | B | A |
| GS-749 | B | A | B | A |
| GS-750 | A | A | A | A |
| GS-751 | A | A | B | A |
| GS-752 | A | A | A | A |
| GS-753 | B | A | C | B |
| GS-754 | D | C | D | C |
| GS-755 | B | A | B | A |
| GS-756 | D | D | D | D |
| GS-757 | D | D | D | D |
| GS-758 | D | D | D | C |
| GS-759 | C | B | D | B |
| GS-760 | D | D | D | D |
| GS-761 | D | C | D | C |
| GS-762 | D | D | D | D |
| GS-763 | B | A | B | A |
| GS-764 | A | A | A | A |
| GS-765 | D | C | D | C |
| GS-766 | A | A | A | A |
| GS-767 | C | B | C | B |
| GS-768 | A | A | A | A |
| GS-769 | A | A | A | A |
| GS-770 | B | B | B | B |
| GS-771 | B | A | B | B |
| GS-772 | B | B | B | A |
| GS-773 | A | A | B | A |
| GS-774 | C | B | D | C |
| GS-775 | C | C | D | C |
| GS-776 | B | A | B | A |
| GS-777 | A | A | B | A |
| GS-778 | B | A | B | A |
| GS-779 | A | A | A | A |
| GS-780 | B | A | B | A |
| GS-781 | B | A | C | B |
| GS-782 | A | A | B | A |
| GS-783 | B | B | B | B |
| GS-784 | C | B | C | C |
| GS-785 | A | A | B | A |
| GS-786 | A | A | B | A |
| GS-787 | D | D | D | D |
| GS-788 | A | A | A | A |
| GS-789 | A | A | A | A |
| GS-790 | A | A | B | A |
| GS-791 | A | A | B | B |
| GS-800 | — | — | A | — |
| GS-801 | — | — | B | — |
| GS-802 | — | — | B | — |
| GS-803 | — | — | A | — |

The IC₅₀ values (nM) of each compound were determined in MV4;11 and MOLM-13 cells with or without gilteritinib (10 nM for MV4;11; and 20 nM for MOML-13) as described in the Materials and Methods. The IC₅₀ values (nM) of each compound were determined in MV4;11 and MOLM-13 cells with or without gilteritinib (10 nM for MV4;11; and 20 nM for MOML-13) as described in Methods. A: <10 nM; 100 nM>B>=10 nM; 1 μM>C>=100 nM; D>=1 μM.

The cell viability inhibition results of selected compounds are set forth in Table 3. The compounds were tested in HEL, 22RV1, NCI-H82 or NCI-H1155 cells.

TABLE 3
Exemplary results in HEL, 22RV1,
NCI-H82 and NCI-H1155 cells.

| | HEL | 22RV1 | NCI-H82 | NCI-H115 |
|---|---|---|---|---|
| | IC₅₀ (nM) | IC₅₀ (nM) | IC₅₀ (nM) | IC₅₀ (nM) |
| GS-800 | B | A | A | — |
| GS-802 | — | — | — | B |
| GS-803 | — | — | — | A |
| GS-804 | C | — | — | — |
| GS-805 | A | — | — | — |
| GS-806 | B | — | — | — |
| GS-807 | A | A | A | — |
| GS-808 | — | — | A | — |
| GS-809 | — | — | — | A |
| GS-810 | — | — | — | B |
| GS-811 | — | — | — | B |
| GS-812 | — | — | A | — |
| GS-813 | — | A | — | — |
| GS-814 | — | A | — | — |

The IC₅₀ values (nM) of each compound were determined in HEL, 22RV1, NCI-H82 and NCI-H1155 cells as described in the Materials and Methods. The IC₅₀ values (nM) of each compound were determined as described in Methods. A:<10 nM; 100 nM>B>=10 nM; 1 μM>C>=100 nM; D>=1 μM.

### Example 166. Pre-clinical studies including a first compound comprising a GSPT1 degrader and a second compound comprising an FLT3 pathway inhibitor

Compounds will be tested in a pre-clinical animal study to further assess the efficacy of some compounds described herein. For example, compounds selected from Table 1, may be used in the pre-clinical animal study.

A mammalian cancer model will be used in these experiments. In short, immune compromised mice will be injected with cancer cells. The cells may include RS4;11, MV4;11, or MOLM-13 cells. The cancer model will include a GSPT1-mediated cancer. Upon injection with cancer cells, mice will receive multiple doses of the compounds with or without an FLT3 pathway inhibitor that includes gilteritinib. Cancer cells will be quantified and assessed periodically after injection into the mice. Assessments of animal and cancer cell health will be carried out. At the end of the study, animals will be sacrificed, and cancer cells and blood will be assayed for various biomarkers and cancer indices. Experiments will include measures of apoptosis, cell death, GSPT1 protein and mRNA levels.

It is expected that treatment with each of the tested compounds will be effective in treating the cancer in the mice. It is also expected that the combination treatment that includes a GSPT1-degrader compound in combination with gilteritinib will result in better cancer outcomes than a placebo control treatment or than either treatment alone. Additional tests may confirm positive effects of combination treatments with other FLT3 pathway inhibitors, or with inhibitors or modulators of other cell signaling pathways such as those described herein.

### Example 167. Clinical Trials Including a First Compound Comprising a GSPT1 Degrader and a Second Compound Comprising an FLT3 Pathway Inhibitor

Compound(s) that perform best in pre-clinical experiments will each be tested in a human clinical trial to evaluate their safety and efficacy in combination with gilteritinib in subjects with acute myeloid leukemia (AML). The compounds to be tested may include one or more compounds from Table 1. The compounds are to be administered to cancer patients alone or in combination with gilteritinib. Indices of cancer growth and severity are determined over time. It is expected that treatment with each of the compounds to be tested will be effective in treating the cancer in patients. It is also expected that the combination treatment that includes a compound to be tested in combination with gilteritinib will result in better cancer outcomes than a placebo control treatment or than either treatment alone.

### Study Description:

The study will be an open-label, multi-arm, parallel multi-cohort, multicenter, study to determine the safety, tolerability, PK, and efficacy of the compounds to be tested in combination with another anti-leukemia agent used for the treatment of AML. The compounds to be tested will be given as a combination therapy to subjects with newly diagnosed (ND) or relapsed or refractory (R/R) AML.

The dose and schedule finding part (Part A) of the study will evaluate the safety, PK and PD data, and preliminary efficacy information and determine the Part B dose and schedule.

The expansion part (Part B) of the study will further evaluate the safety and efficacy of the compounds to be tested alone and combined with gilteritinib, at or below the maximum tolerated dose (MTD) in the selected cohorts in order to determine the recommended Phase 2 dose (RP2D) for subjects with AML.

### Experimental:

The compounds to be tested will each be administered intravenously per a dosing schedule in a 28-day cycle. Gilteritinib will be administered orally QD.

### Primary Outcome Measures:

1. Dose Limiting Toxicity (DLT) [Time Frame: Up to 28 days]
   Number of participants with a DLT
2. Maximum Tolerated Dose (MTD) [Time Frame: Up to 28 days.]
   The highest dose with DLT rate in Cycle 1 being lower than or close to the target level 0.3 and
   the toxicity probability within (0.25, 0.35) interval.
3. Adverse Events (AEs) [Time Frame: Up to 28 days after last dose of study drug.]
An AE is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values, regardless of etiology.

### Secondary Outcome Measures:

1. Complete Remission Rate (CRR), [Time Frame: Up to 3 years]
   is defined as the rate for any type of CR or CRh
2. Objective Response Rate (ORR) [Time Frame: Up to 3 years]
   is defined as the rate for all types of CRs and PR for AML.
3. Progression Free Survival (PFS) [Time Frame: Up to 3 years]
   is defined as the time from the first dose of study drug(s) to the first occurrence of relapse or progression or death from any cause
4. Overall Survival (OS) [Time Frame: Up to 3 years]
   is measured as the time from the first dose of study drug(s) to death due to any cause and will be analyzed in a manner similar to that described for PFS.
5. Duration of Remission [Time Frame: Up to 3 years]
   is measured from the time when criteria for CR/CRh/PR are first met (whichever is first recorded) until the first date at which relapse, or progressive disease is objectively documented.
6. Time to Remission [Time Frame: Up to 3 years]
   is measured from the time when criteria for CR/CRh/PR are first met (whichever is first recorded)
7. Pharmacokinetics—Cmax [Time Frame: Until last dose of the compounds to be tested in Cycle 3 (each cycle is 28 days. Dosing days for the compounds to be tested are Days 1-5 in Cycle 3)]
   observed maximum concentration in plasma
8. Pharmacokinetics—AUC24 [Time Frame: Until last dose of the compounds to be tested in Cycle 3 (each cycle is 28 days. Dosing days for the compounds to be tested are Days 1-5 in Cycle 3)]
   area under the plasma concentration time-curve from time 0 to 24 h postdose
9. Pharmacokinetics—t1/2 [Time Frame: Until last dose of the compounds to be tested in Cycle 3 (each cycle is 28 days. Dosing days for the compounds to be tested are Days 1-5 in Cycle 3)] terminal half life

### Inclusion Criteria:

1. Adult subject must understand and voluntarily sign an ICF prior to any study-related assessments/procedures being conducted.
2. Subject is ≥18 years of age.
3. FLT3-ITD positive relapsed or refractory AML.
4. Gilteritinib treatment na [symbol] ve.
5. Subject has Eastern Cooperative Oncology Group (ECOG) performance status of 0, 1 or 2.
6. Subject must have the following screening laboratory values:
   a. Total White Blood Cell count (WBC)<25×10\hat{ }9 prior to study treatments. Treatment with hydroxyurea to achieve this level is allowed.
   b. Selected electrolytes within normal limits or correctable with supplements.
      Participant must have adequate liver function as demonstrated by: Aspartate aminotransferase (AST) and alanine aminotransferase (ALT)≤2.5× upper limit of normal (ULN) and bilirubin≤1.5×ULN
      Participant has adequate renal function as demonstrated by an estimated serum creatinine clearance of ≥60 mL/min using the Cockcroft-Gault equation.
7. Agree to follow a Pregnancy Prevention Plan (PPP) and combination agents' requirements

### Exclusion Criteria:

1. Subject with acute promyelocytic leukemia (APL)
2. Subject has received systemic anticancer therapy (including investigational therapy) or radiotherapy <28 days or 5 half-lives, whichever is shorter, prior to the start of study treatment
3. Patients with prior autologous hematopoietic stem cell transplant (HSCT) who, in the investigator's judgment, have not fully recovered from the effects of the last transplant (e.g., transplant related side effects)
4. Prior allogeneic HSCT with either standard or reduced intensity conditioning ≤6 months prior to dosing
5. Subject on systemic immunosuppressive therapy post HSCT at the time of screening, or with clinically significant graft-versus-host disease (GVHD). The use of topical steroids for ongoing skin or ocular GVHD is permitted
6. Subject has persistent, clinically significant non-hematologic toxicities from prior therapies which have not recovered to <Grade 2
7. Subject has or is suspected of having central nervous system (CNS) leukemia. Evaluation of cerebrospinal fluid is only required if CNS involvement by leukemia is
8. Impaired cardiac function or clinically significant cardiac diseases, including any of the following:
   a. Left ventricular ejection fraction (LVEF)<45% as determined by multiple gated acquisition (MUGA) scan or echocardiogram (ECHO).
   b. Complete left bundle branch or bifascicular block.
   c. Congenital long QT syndrome.
   d. Persistent or clinically meaningful ventricular arrhythmias.
   e. QTcF>450 ms on Screening electrocardiogram (ECG)
   f. Unstable angina pectoris or myocardial infarction ≤6 months prior to starting study treatments or unstable arrhythmia.
   g. Cardiovascular disability status of New York Heart Association Class ≥2. Class 2 is defined as cardiac disease in which patients are comfortable at rest but ordinary physical activity results in fatigue, palpitations, dyspnea, or anginal pain suspected during screening.
9. Subject is a pregnant or lactating female

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (A): 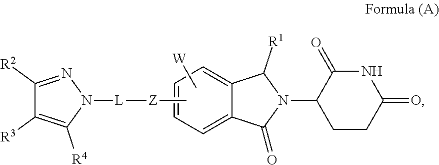
or a pharmaceutically acceptable salt thereof, wherein:
W is hydrogen or fluorine;
Z is absent, —NR^{1a}—, or —O—; wherein R^{1a} is hydrogen or C₁-C₈ alkyl;
L is an optionally substituted C₁-C₁₀ alkylene or optionally substituted C₁-C₁₀ heteroalkylene, or
L is 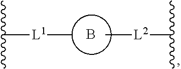
wherein:
Ring B is an optionally substituted 3-7 membered carbocyclyl or optionally substituted 4-7 membered heterocyclyl;
L¹ is absent, or an optionally substituted C₁-C₁₀ alkylene or optionally substituted C₁-C₁₀ heteroalkylene;
L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl, optionally substituted 6-10 membered aryl, or optionally substituted 5-10 membered heteroaryl; or
R² and R³ or R³ and R⁴ are taken together to form an optionally substituted 3-7 membered partially saturated or unsaturated carbocyclyl, optionally substituted 4-7 membered partially saturated or unsaturated heterocyclyl, optionally substituted phenyl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl or optionally substituted 5-6 membered heteroaryl ring.

2. A compound of Formula (I): 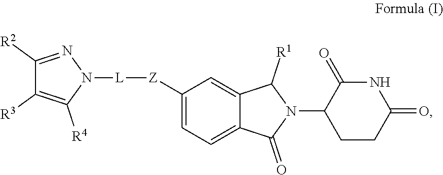
or a pharmaceutically acceptable salt thereof, wherein:
Z is absent, —NR^{1a}—, or —O—; wherein R^{1a} is hydrogen or C₁-C₈ alkyl;
L is an optionally substituted C₁-C₁₀ alkylene or optionally substituted C₁-C₁₀ heteroalkylene, or
L is 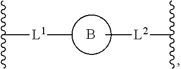
wherein:
Ring B is an optionally substituted 3-7 membered carbocyclyl or optionally substituted 4-7 membered heterocyclyl;
L¹ is absent, or an optionally substituted C₁-C₁₀ alkylene or optionally substituted C₁-C₁₀ heteroalkylene;
L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene;
R¹ is absent or oxo (═O);
R² and R⁴ are each independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl;
R³ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl, optionally substituted 6-10 membered aryl, or optionally substituted 5-10 membered heteroaryl; or
R² and R³ or R³ and R⁴ are taken together to form an optionally substituted partially unsaturated 3-7 membered carbocyclyl, optionally substituted partially unsaturated 4-7 membered heterocyclyl, optionally substituted phenyl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted 6-10 membered aryl, or optionally substituted 5-10 membered heteroaryl; or
R⁵ and R⁶ together with the atom(s) to which they are attached optionally form an optionally substituted 4-7 membered heterocyclyl, or optionally substituted 5-6 membered heteroaryl ring.

3. The compound of claim 1 or claim 2, or a pharmaceutically acceptable salt thereof, wherein R² and R³ are taken together to form an optionally substituted phenyl or optionally substituted 5-6 membered heteroaryl ring.

4. The compound of claim 1 or claim 2, or a pharmaceutically acceptable salt thereof, wherein R³ and R⁴ are taken together to form an optionally substituted phenyl or optionally substituted 5-6 membered heteroaryl ring.

5. The compound of claim 1, 2, or 4, wherein the compound has the structure of Formula (II): 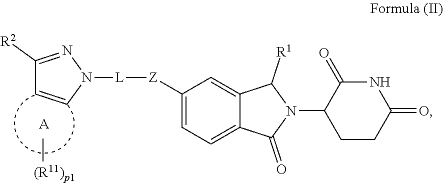
or a pharmaceutically acceptable salt thereof, wherein:
Ring A is a phenyl or 5-6 membered heteroaryl;
each R¹¹ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; and
p₁ is 0, 1, 2, 3, or 4.

6. The compound of claim 5, or a pharmaceutically acceptable salt thereof, wherein Ring A is phenyl, pyridinyl, or triazinyl.

7. The compound of claim 5, wherein the compound has the structure of Formula (II-A): 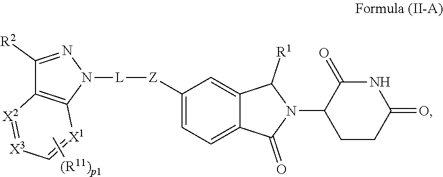
or a pharmaceutically acceptable salt thereof, wherein:
X¹, X² and X³ are each independently CR¹¹ or N.

8. The compound of claim 7, or a pharmaceutically acceptable salt thereof, wherein X¹ is N; and X² and X³ are each independently CR¹¹.

9. The compound of claim 7, or a pharmaceutically acceptable salt thereof, wherein X¹ and X² are each N; and X³ is CR¹¹.

10. The compound of claim 7, or a pharmaceutically acceptable salt thereof, wherein X¹ and X³ are each N; and X² is CR¹¹.

11. The compound of claim 1 or 2, or a pharmaceutically acceptable salt thereof, wherein R⁴ is hydrogen; and R³ is N(R⁵)R⁶, optionally substituted 3-10 membered carbocyclyl, optionally substituted 3-10 membered heterocyclyl, optionally substituted 6-10 membered aryl, or optionally substituted 5-10 membered heteroaryl.

12. The compound of claim 11, or a pharmaceutically acceptable salt thereof, wherein R³ is optionally substituted phenyl or optionally substituted 5-10 membered heteroaryl.

13. The compound of any one of claims 1, 2, 11 or 12, wherein the compound has the structure of Formula (III): 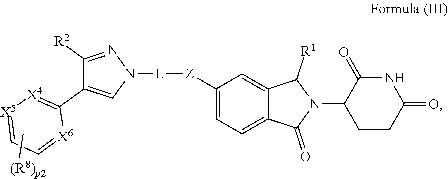
or a pharmaceutically acceptable salt thereof, wherein:
X⁴, X⁵ and X⁶ are each independently CR⁸ or N;
each R⁸ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
two R⁸ on adjacent carbon atoms are taken together to form an optionally substituted partially unsaturated 3-7 membered carbocyclyl, optionally substituted partially unsaturated 4-7 membered heterocyclyl, optionally substituted phenyl, or optionally substituted 5-6 membered heteroaryl ring; and
p₂ is 0, 1, 2, 3, 4, or 5.

14. The compound of claim 13, or a pharmaceutically acceptable salt thereof, wherein X⁴, X⁵ and X⁶ are each independently CR⁸.

15. The compound of claim 13, or a pharmaceutically acceptable salt thereof, wherein X⁴ is N; and X⁵ and X⁶ are each independently CR⁸.

16. The compound of any one of claims 13 to 15, or a pharmaceutically acceptable salt thereof, wherein each R⁸ is independently hydrogen, halogen, or optionally substituted C₁-C₈ alkyl.

17. The compound of any one of claims 1, 2, 11 or 12, wherein the compound has the structure of Formula (IV): 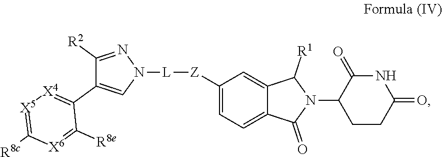
or a pharmaceutically acceptable salt thereof, wherein:
X⁴ is CR^{8a} or N;
X⁵ is CR^{8b} or N;
X⁶ is CR^{8d} or N;
R^{8a}, R^{8b}, R^{8c}, R^{8d}, and R^{8e} are each independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; or
R^{8a} and R^{8b}, R^{8b} and R^{8c}, R^{8c} and R^{8d}, or R^{8d} and R^{8e} are taken together with the atoms to which they are attached to form an optionally substituted 3-7 membered carbocyclyl, optionally substituted 4-7 membered heterocyclyl, optionally substituted 6 membered aryl, or optionally substituted 5-6 membered heteroaryl ring.

18. The compound of claim 17, or a pharmaceutically acceptable salt thereof, wherein X⁴ is N, X⁵ is CR^{8b}, and X⁶ is CR^{8d}.

19. The compound of claim 17 or 18, or a pharmaceutically acceptable salt thereof, wherein R^{8b}, R^{8c}, R^{8d}, and R^{8e} are each independently hydrogen, halogen, or optionally substituted C₁-C₈ alkyl.

20. The compound of claim 17, wherein the compound has the structure of Formula (IV-A): 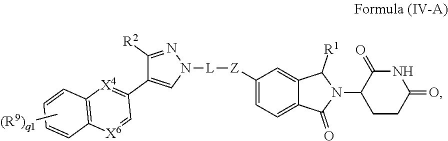
or a pharmaceutically acceptable salt thereof, wherein:
each R⁹ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl; and
q₁ is 0, 1, 2, or 3.

21. The compound of claim 17, wherein the compound has the structure of Formula (IV-B): 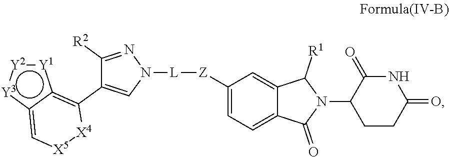
or a pharmaceutically acceptable salt thereof, wherein:
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

22. The compound of claim 21, or a pharmaceutically acceptable salt thereof, wherein X⁴ is N and X⁵ is CR^{8b}.

23. The compound of claim 17, wherein the compound has the structure of Formula (IV-C): 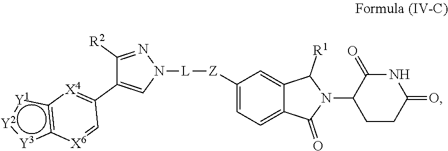
or a pharmaceutically acceptable salt thereof, wherein:
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

24. The compound of claim 23, or a pharmaceutically acceptable salt thereof, wherein X⁴ is N and X⁶ is CR^{8d}.

25. The compound of claim 23, or a pharmaceutically acceptable salt thereof, wherein X⁴ is CR^{8a} and X⁶ is N.

26. The compound of claim 17, wherein the compound has the structure of Formula (IV-D): 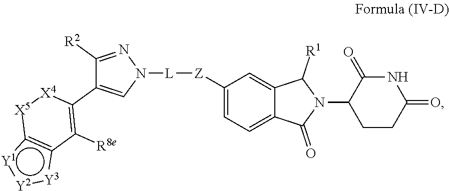
or a pharmaceutically acceptable salt thereof, wherein:
Y¹, Y², and Y³ are each independently CR¹⁰, NR^{10a}, or N; wherein at least one of Y¹, Y², and Y³ is CR¹⁰;
each R¹⁰ is independently hydrogen, halogen, CN, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, optionally substituted 3-10 membered heterocyclyl; and
each R^{10a} is independently hydrogen, optionally substituted C₁-C₈ alkyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, optionally substituted 3-10 membered heterocyclyl.

27. The compound of claim 26, or a pharmaceutically acceptable salt thereof, wherein X⁴ is N and X⁵ is CR^{8b}.

28. The compound of claim 26, or a pharmaceutically acceptable salt thereof, wherein X⁴ is CR^{8a} and X⁵ is N.

29. The compound of claim 26, or a pharmaceutically acceptable salt thereof, wherein X⁴ is N and X⁵ is N.

30. The compound of any one of claims 21 to 29, or a pharmaceutically acceptable salt thereof, wherein Y¹ and Y³ are each independently NR^{10a} or N; and Y² is CR¹⁰.

31. The compound of any one of claims 21 to 29, or a pharmaceutically acceptable salt thereof, wherein Y² and Y³ are each independently NR^{10a} or N; and Y¹ is CR¹⁰.

32. The compound of any one of claims 21 to 29, or a pharmaceutically acceptable salt thereof, wherein Y¹ and Y² are each independently NR^{10a} or N; and Y³ is CR¹⁰.

33. The compound of any one of claims 1 to 32, or a pharmaceutically acceptable salt thereof, wherein Z is absent or —O—.

34. The compound of any one of claims 1 to 32, or a pharmaceutically acceptable salt thereof, wherein Z is —NR^{1a}—.

35. The compound of claim 34, or a pharmaceutically acceptable salt thereof, wherein Z is —NH—.

36. The compound of any one of claims 1 to 35, or a pharmaceutically acceptable salt thereof, wherein:
L is 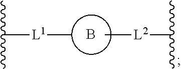
wherein
Ring B is an optionally substituted 3-7 membered carbocyclyl, or optionally substituted 4-7 membered heterocyclyl;
L¹ is absent, or an optionally substituted C₁-C₁₀ alkylene or optionally substituted C₁-C₁₀ heteroalkylene; and
L² is absent, or an optionally substituted C₁-C₁₀ alkylene, optionally substituted C₂-C₁₀ alkenylene, optionally substituted C₂-C₁₀ alkynylene, or optionally substituted C₁-C₁₀ heteroalkylene.

37. The compound of claim 36, or a pharmaceutically acceptable salt thereof, wherein:
Ring B is an optionally substituted 3-7 membered carbocyclyl;
L¹ is absent or an optionally substituted C₁-C₁₀ alkylene; and
L² is absent or an optionally substituted C₁-C₁₀ alkylene.

38. The compound of claim 36 or claim 37, or a pharmaceutically acceptable salt thereof, wherein:
L¹ is absent; and
L² is C₁-C₁₀ alkylene.

39. The compound of any one of claims 36 to 38, or a pharmaceutically acceptable salt thereof, wherein Ring B is an optionally substituted cyclopropyl, cyclobutyl, cyclopentyl, cyclohexyl, spiro[3.3]heptyl, spiro[4.4]nonyl, or spiro[3.4]octanyl ring.

40. The compound of any one of claims 1 to 39, or a pharmaceutically acceptable salt thereof, wherein R² is halogen, CN, OR⁵, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl.

41. The compound of claim 40, or a pharmaceutically acceptable salt thereof, wherein R² is an optionally substituted 3-10 membered carbocyclyl.

42. The compound of any one of claims 1 to 41, or a pharmaceutically acceptable salt thereof, wherein R¹ is absent.

43. The compound of any one of claims 1 to 41, or a pharmaceutically acceptable salt thereof, wherein R¹ is oxo (═O).

44. A pharmaceutical composition comprising a compound of any one of claims 1 to 43, or a pharmaceutically acceptable salt thereof, and a pharmaceutically acceptable excipient.

45. A method for the treatment of abnormal cell growth in a subject in need thereof, comprising administering to the subject an effective amount of the compound of any one of claims 1 to 43, or a pharmaceutically acceptable salt thereof, or the composition of claim 40.

46. The method of claim 45, wherein the subject has cancer.

47. The method of claim 45 or claim 46, further comprising administering to the subject a second compound comprising an FLT3 pathway inhibitor, a RAS-RAF-MEK-ERK pathway inhibitor, or a PI3K-AKT-mTOR pathway inhibitor or activator.

48. The method of claim 47, wherein the FLT3 pathway inhibitor is gilteritinib, midostaurin, sorafenib, sunitinib, lestaurtinib, quizartinib, crenolanib or sitravatinib, or a pharmaceutically acceptable salt thereof.

49. The method of claim 48, wherein the FLT3 pathway inhibitor is gilteritinib, or a pharmaceutically acceptable salt thereof.

50. A compound of any one of claims 1 to 43, or a pharmaceutically acceptable salt thereof, for use in the treatment of cancer.

51. A compound of any one of claims 1 to 43, or a pharmaceutically acceptable salt thereof, for use in the manufacture of a medicament for the treatment of cancer.

52. A combination comprising a compound of any one of claims 1 to 43, or a pharmaceutically acceptable salt thereof, and a second compound comprising an FLT3 pathway inhibitor, a RAS-RAF-MEK-ERK pathway inhibitor, or a PI3K-AKT-mTOR pathway inhibitor or activator.

53. The combination of claim 52, wherein the FLT3 pathway inhibitor is gilteritinib, midostaurin, sorafenib, sunitinib, lestaurtinib, quizartinib, crenolanib or sitravatinib, or a pharmaceutically acceptable salt thereof.

54. The method of claim 52 or claim 53, wherein the FLT3 pathway inhibitor is gilteritinib, or a pharmaceutically acceptable salt thereof.
